(19) United States
(12) Patent Application Publication
(10) Pub. No.: US 2024/0425831 A1
(43) Pub. Date: Dec. 26, 2024

(54) IMPROVED CRISPR PRIME EDITORS

(71) Applicant: The General Hospital Corporation, Boston, MA (US)

(72) Inventors: J. Keith Joung, Winchester, MA (US); Julian Grünewald, Charlestown, MA (US); Bret Miller, Niceville, FL (US)

(21) Appl. No.: 18/699,164

(22) PCT Filed: Oct. 7, 2022
(86) PCT No.: PCT/US2022/077789

§ 371 Date: Apr. 5, 2024

Related U.S. Application Data
(60) Provisional application No. 63/408,406, filed on Sep. 20, 2022.
(60) Provisional application No. 63/253,948, filed on Oct. 8, 2021.

(51) Int. Cl. C12N 9/22; C12N 9/12; C12N 15/11; C12N 15/90

(52) U.S. Cl. CPC C12N 9/22; C12N 9/1276; C12N 15/11; C12N 15/907; C12Y 207/07049; C07K 2319/50; C12N 2310/20

(57) ABSTRACT

Described herein are split and reduced size CRISPR Prime Editors, as well as variant reverse transcriptases, and methods of use thereof.

### CLAIM OF PRIORITY

This application claims the benefit of U.S. Patent Application Ser. No. 63/253,948, filed on Oct. 8, 2021, and 63/408,406, filed on Sep. 20, 2022. The entire contents of the foregoing are hereby incorporated by reference.

### FEDERALLY SPONSORED RESEARCH OR DEVELOPMENT

This invention was made with Government support under Grant Nos. HG009490 and GM118158 awarded by the National Institutes of Health. The Government has certain rights in the invention.

### TECHNICAL FIELD

Described herein are split and reduced size CRISPR Prime Editors, as well as variant reverse transcriptases, and methods of use thereof.

### BACKGROUND

CRISPR prime editors (PEs) use RNA-guided reverse transcription to mediate programmable introduction of a wide range of genetic alterations¹, but the large sizes of PE proteins can create challenges for research and therapeutic applications. The most commonly used PE protein, commonly referred to as PE2, is composed of a CRISPR Streptococcus pyogenes Cas9 nickase (nSpCas9) with a pentamutant (D200N/L603W/T330P/T306K/W313F) Moloney Murine Leukemia Virus reverse transcriptase (MMLV-RT) fused at its C-terminus^{1, 30, 31}.

### SUMMARY

As shown herein, fully separated nSpCas9 and MMLV-RT functioned together as efficiently as intact PE2 in human cells, suggesting that the MMLV-RT enzyme acts in trans (i.e., untethered to DNA) rather than in cis to nSpCas9. A similarly split version of Staphylococcus aureus Cas9 nickase² (nSaCas9)-based PE2 protein exhibited activity comparable to the intact fusion. This separability was exploited to rapidly identify alternative RTs with potentially desirable characteristics, including a reduced-size MMLV-RT variant lacking any RNase H domain with activity equivalent to its full-length parent and an even smaller size engineered group II intron maturase RT domain from Eubacterium rectale, as well as Geobacillus stearothermophilus GsI-IIC intron RT (GsI-IIC RT) and human endogenous retrovirus K (e.g., HERV-Kcon; derived consensus sequence), that can induce prime editing in human cells. The split PE and reduced size PE architectures described herein provide advantages and improved optionality for delivery, expression, and purification of prime editing components. More broadly, these findings further define the mechanism of prime editing and provide a simplified framework for higher throughput development of novel PE designs with improved and/or altered properties.

Thus, provided herein are compositions comprising (a) a Cas nickase protein and a reverse transcriptase (RT) protein, wherein the Cas nickase and RT are separate molecules, i.e., are not tethered, conjugated, or fused together, as described herein, or (b) a fusion protein comprising a Cas nickase protein linked to a reverse transcriptase (RT) protein, with a cleavable linker between the Cas nickase and the RT, optionally wherein the cleavable linker is a 2A self-cleaving peptide or protease-cleavable linker.

Also provided herein are compositions comprising (i) a nucleic acid comprising a sequence encoding a Cas nickase protein and (ii) a nucleic acid comprising a sequence encoding a reverse transcriptase (RT) protein, wherein the Cas nickase and RT are encoded as separate molecules, i.e., are not tethered, conjugated, or fused together, optionally wherein each nucleic acid is in a separate expression vector (e.g., a viral vector, e.g., an AAV), are expressed as separate cassettes within a single expression vector. As one example, two expression vectors (e.g., AAV) can be used, e.g., wherein one vector can include a nucleic acid comprising a sequence encoding a Cas nickase protein, but no RT sequences, and a second vector can include a nucleic acid comprising a sequence encoding a reverse transcriptase (RT) protein but no Cas sequences; one or both can include sequences encoding a pegRNA and/or ngRNA. In some embodiments, a single expression vector can include sequences for separate expression of the Cas nickase and RT, wherein the Cas nickase and RT are encoded and expressed as entirely separate molecules. The nucleic acids can also be cDNA or mRNA. Alternatively, the Cas nickase and RT are expressed as a fusion protein that is cleaved into separate Cas nickase protein and RT protein components following their expression as a single polypeptide (e.g., with the components separated by a protease cleavage site or a 2A self-cleaving peptide sequence), e.g., a nucleic acid comprising a sequence encoding a Cas nickase protein in frame with a reverse transcriptase (RT) protein, with a cleavable linker between the Cas nickase and the RT, optionally wherein the cleavable linker is a 2A self-cleaving peptide or protease-cleavable linker, optionally wherein the nucleic acid is in a separate expression vector, e.g., a viral vector, e.g., an AAV.

In some embodiments, the compositions further comprise a pegRNA that can coordinate with the Cas nickase and RT to edit target DNA, optionally in an RNP complex with the Cas protein.

Also provided herein are methods of editing target DNA, e.g., genomic DNA of a cell or DNA in vitro, the method comprising contacting the DNA or cell with, or expressing in the cell, (a) a Cas nickase protein and a reverse transcriptase (RT) protein and a pegRNA that can coordinate with the Cas nickase and RT to edit the target DNA, and optionally an ngRNA, wherein the Cas nickase and RT are separate molecules, i.e., are not tethered, conjugated, or fused together, or (b) a fusion protein comprising a Cas nickase protein linked to a reverse transcriptase (RT) protein, with a cleavable linker between the Cas nickase and the RT, optionally wherein the cleavable linker is a 2A self-cleaving peptide or protease-cleavable linker.

Additionally, provided herein are truncated variant Moloney Murine Leukemia Virus reverse transcriptase (MMLV-RT) proteins lacking any RNase H domain, preferably comprising a deletion of at least 1 and up to 207, 205, 200, 198, 195, 190, 185, or 181 amino acids from the C terminus, and optionally at least 1 and up to 23, 24, or 25 amino acids from the N terminus, and optionally wherein the MMLV-RT comprises mutations D200N/T330P/T306K/W313F and optionally L603W in MMLV-RT. Also provided are isolated nucleic acids encoding the truncated variant MMLV-RT as described herein, optionally wherein the nucleic acid is in an expression vector, e.g., a viral vector, e.g., an AAV.

Additionally, provided herein are GsI-IIC RT pentamutant proteins. Also provided are isolated nucleic acids encoding the GsI-IIC RT pentamutants (e.g., SEQ ID NO:37 comprising mutations D11R/N23R/G71R/G113K/P194R), optionally wherein the nucleic acid is in an expression vector, e.g., a viral vector, e.g., an AAV.

Further provided herein are methods for editing target DNA, e.g., genomic DNA of a cell or DNA in vitro. The methods comprise contacting the DNA or cell with, or expressing in the cell, (i) a Cas nickase protein and (ii) truncated variant MMLV-RT protein as described herein, and a pegRNA that can coordinate with the Cas nickase and RT to edit the target DNA, and optionally an ngRNA, optionally wherein the Cas nickase and RT are separate molecules, or wherein the Cas nickase and RT are tethered, conjugated, or fused together, e.g., wherein the RT is fused to the Cas nickase at the N terminus or C terminus, optionally with a cleavable linker between the Cas nickase and the RT, optionally wherein the cleavable linker is a 2A self-cleaving peptide or protease-cleavable linker, or is inlaid internally (wherein the RT is inlaid internally into the Cas).

Additionally provided herein are variant Eubacterium rectale reverse transcripase (MarathonRT) proteins comprising a mutation as shown herein, e.g., in Table C, preferably wherein the variant has increased prime editing efficiency compared to WT Marathon-RT, preferably wherein the variant comprises mutations at one, two, three, four, or all five of D14, N26, D74, N116, and/or N197, preferably D14R-N26R-D74R-N116K; D14R-D74R-N116K-N197R; D14R-N26R-D74R-N197R; or D14R-N26R-D74R-N116K-N197R, as well as isolated nucleic acids encoding the variant MarathonRTs, optionally wherein the nucleic acid is in an expression vector, e.g., a viral vector, e.g., an AAV.

Also provided herein are proteins and nucleic acid sequences as shown herein, e.g., in any of the tables herein, e.g., in Table C, as well as vectors comprising the nucleic acid sequences, and cells expressing the sequences, and compositions comprising the proteins or nucleic acid sequences.

Further, provided herein are methods of editing target DNA. e.g., genomic DNA of a cell or DNA in vitro. The methods comprise contacting the DNA or cell with, or expressing in the cell, (i) a Cas nickase protein and (ii) a variant MarathonRT protein as described herein, and a pegRNA that can coordinate with the Cas nickase and RT to edit the target DNA, and optionally an ngRNA, wherein the Cas nickase and RT are separate molecules, or wherein the Cas nickase and RT are tethered, conjugated, or fused together, e.g., wherein RT is fused to the Cas nickase at the N terminus or C terminus, optionally with a cleavable linker therebetween, optionally wherein the cleavable linker is a 2A self-cleaving peptide or protease-cleavable linker, or is inlaid internally (wherein the RT is inlaid internally into the Cas).

Also provided herein are prime editor fusion proteins using the variants described herein, e.g., comprising: (i) a Cas9 nickase protein tethered, conjugated, or fused to a truncated variant MMLV-RT as described herein, a variant MarathonRT protein as described herein, or a wild type RT selected from MarathonRT, Human Endogenous Retrovirus K consensus sequence (HERV-Kcon RT), and Geobacillus stearothermophilus GsI-IIC intron RT (GsI-IIC RT), optionally with a cleavable linker therebetween, optionally wherein the cleavable linker is a 2A self-cleaving peptide or protease-cleavable linker, or (ii) a Cas9 nickase protein comprising the truncated variant MMLV-RT as described herein, the variant MarathonRT protein as described herein, a MMLV-RT pentamutant (e.g., as described in Anzalone et al.) or Geobacillus stearothermophilus GsI-IIC intron RT (GsI-IIC RT) pentamutant, or a wild type RT selected from MarathonRT, Human Endogenous Retrovirus K consensus sequence (HERV-Kcon RT), and Geobacillus stearothermophilus GsI-IIC intron RT (GsI-IIC RT), wherein the MMLV-RT is inlaid into the Cas9 nickase, optionally wherein the MMLV is inlaid at G1247 or G1055 (i.e., between G1247/S1248 or G1055/E1056), as described herein.

Also provided are nucleic acids encoding the prime editor fusion proteins as described herein, optionally wherein the nucleic acid is in an expression vector, e.g., a viral vector, e.g., an AAV.

Also provided are compositions comprising the prime editor fusion proteins as described herein, or a nucleic acid encoding a prime editor fusion protein as described herein, and a pegRNA, and optionally an ngRNA.

Additionally, provided herein are compositions comprising: (i) a Cas9 nickase protein and (ii) an RT, wherein the RT comprises a truncated variant MMLV-RT as described herein, a MMLV-RT pentamutant or GsI-IIC RT pentamutant as described herein, a variant MarathonRT protein as described herein, or a wild type RT selected from MarathonRT, Human Endogenous Retrovirus K consensus sequence (HERV-Kcon RT), and GsI-IIC RT, wherein the Cas nickase and RT are separate molecules, i.e., are not tethered, conjugated, or fused together. Alternatively, the Cas nickase and RT are expressed as a fusion protein that is cleaved into separate Cas nickase protein and RT protein components following their expression as a single polypeptide (e.g., with the components separated by a protease cleavage site or a 2A self-cleaving peptide sequence), e.g., a nucleic acid comprising a sequence encoding a Cas nickase protein in frame with a reverse transcriptase (RT) protein, with a cleavable linker between the Cas nickase and the RT, optionally wherein the cleavable linker is a 2A self-cleaving peptide or protease-cleavable linker, optionally wherein the nucleic acid is in a separate expression vector, e.g., a viral vector, e.g., an AAV.

Further provided are compositions comprising (i) a nucleic acid comprising a sequence encoding a Cas nickase protein and (ii) a nucleic acid comprising a sequence encoding an RT, wherein the RT comprises a truncated variant MMLV-RT as described herein, a MMLV-RT pentamutant or GsI-IIC RT pentamutant as described herein, a variant MarathonRT protein as described herein, or a wild type RT selected from MarathonRT, Human Endogenous Retrovirus K consensus sequence (HERV-Kcon RT), and GsI-IIC RT, wherein the Cas nickase and RT are encoded as separate molecules, i.e., are not tethered, conjugated, or fused together, optionally wherein each nucleic acid is in a separate expression vector, e.g., a viral vector, e.g., an AAV. Alternatively, the Cas nickase and RT are expressed as a fusion protein that is cleaved into separate Cas nickase protein and RT protein components following their expression as a single polypeptide (e.g., with the components separated by a protease cleavage site or a 2A self-cleaving peptide sequence), e.g., a nucleic acid comprising a sequence encoding a Cas nickase protein in frame with a reverse transcriptase (RT) protein, optionally with a cleavable linker between the Cas nickase and the RT, optionally wherein the cleavable linker is a 2A self-cleaving peptide or protease-cleavable linker, optionally wherein the nucleic acid is in a separate expression vector, e.g., a viral vector, e.g., an AAV.

The compositions described herein can be used, e.g. in methods of editing target DNA. Thus also provided herein are methods of editing target DNA, e.g., genomic DNA of a cell or DNA in vitro, the method comprising contacting the DNA or cell with, or expressing in the cell, (i) a Cas nickase protein and (ii) an RT, wherein the RT comprises a truncated variant MMLV-RT as described herein, a MMLV-RT pentamutant or GsI-IIC RT pentamutant as described herein, a variant MarathonRT protein as described herein, or a wild type RT selected from MarathonRT, Human Endogenous Retrovirus K consensus sequence (HERV-Kcon RT), and GsI-IIC RT, wherein the Cas nickase and RT are separate molecules, i.e., are not tethered, conjugated, or fused together, and a pegRNA that can coordinate with the Cas nickase and RT to edit the target DNA, and optionally an ngRNA, wherein the Cas nickase and RT are separate molecules, or wherein the Cas nickase and RT are tethered, conjugated, or fused together, e.g., wherein RT is fused to the Cas nickase at the N terminus or C terminus, optionally with a cleavable linker between the Cas nickase and the RT, optionally wherein the cleavable linker is a 2A self-cleaving peptide or protease-cleavable linker, or wherein the RT is inlaid internally into the Cas (wherein the RT is inlaid internally into the Cas).

In any of the compositions or methods described herein, the Cas nickase can a nickase shown in Table A1, or a variant thereof, e.g., as shown in Table A2, e.g., wherein the Cas nickase is Cas9, preferably from S. pyogenes (nSpCas9, e.g., comprising mutations H840. D839A, or N863A) or S. aureus (nSaCas9, e.g. comprising mutations D10A or N580). In some embodiments, the Cas nickase is nSaCas9. Although the Cas referred to above is a Cas nickase, Cas nucleases can also be used in the present methods and compositions.

Further, provided herein are methods of transcribing RNA into DNA in vitro or in a cell or tissue, the method comprising contacting the RNA with an RT, wherein the RT comprises a truncated variant MMLV-RT as described herein, a GsI-IIC RT pentamutant as described herein, a variant MarathonRT protein as described herein, and sufficient nucleotides to transcribe DNA (as well as other factors necessary for the reaction to run). For methods in which a cell or tissue is used, the methods can further include expressing the RT in the cell or tissue.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-C. Schematic overview of prime editing. A, The PE2 protein consists of Streptococcus pyogenes Cas9 (H840) nickase (nSpCas9 in grey; silhouette derived from PDB 4OO8) with an MMLV-RT pentamutant domain fused to its C-terminus (light pink; silhouette derived from PDB 4MH8). PE2 is programmed to target a genomic locus of interest with a pegRNA. An R-loop is formed upon binding of the PE-pegRNA ribonucleoprotein (RNP) to the protospacer on the target strand (TS) on DNA. nSpCas9 introduces a nick (grey circle) on the non-target strand (NTS). The 3′ extension consists of a primer binding site (PBS) and a reverse transcription template (RTT). B, The PBS of the pegRNA anneals to the NTS upstream of where the nick was introduced. C, The RT domain extends a single-stranded 3 DNA flap from the nicked NTS using the RTT which encodes the desired edit. For the PE3 strategy, a second gRNA (ngRNA) nicks the TS (opposite the 3′ flap) up- or downstream of the prime editing target site. The illustration is adapted from Supplementary FIG. 1a-c of Hsu et al.²⁵.

FIGS. 1D-G. Split and intact (also referred to as fused) prime editors function with comparable efficiencies in human HEK293T cells. D, Schematic illustrating the location of MMLV-RT (grey box) with respect to nSpCas9-H840A (white box) for three intact variants (C-terminal, N-terminal, and inlaid fusion at G1247) and the separate expression of nSpCas9 and the MMLV-RT pentamutant for Split-PE (not drawn to scale). Dot and bar plots represent the frequencies of prime editing induced at 11 genomic loci targeted with prime editing gRNAs (pegRNAs) and nicking gRNAs (ngRNAs) using the PE3 approach. The types of desired edits induced are grouped as substitutions (E), insertions (ins., F), or deletions (del., G). Legend shown in E also applies to F and G. For substitution edits, frequencies of pure prime edits (PE), impure PEs (IPE), and byproducts are shown separately. For insertion and deletion edits, IPE and byproduct frequencies are added together and shown as a single bar next to their respective PPE frequencies²³. Bar graphs represent the mean, error bars show standard deviation (s.d.), and dots represent values of replicates (n=3; independent replicates). bp, base pairs. FLAG, Flag tag (DYKDDDDK, SEQ ID NO:120) with insertion size of 33 bp²⁴ with an SGS-linker.

FIG. 1H: Inlaid full-length MMLV-RT pentamutant fusion to nSpCas9 at G1247-S1248 shows efficient prime editing in human HEK293T cells. Prime editing frequencies of a nickase only negative control, a PE3 positive control, and the inlaid MMLV-RT fusion at positions G1247/S1248 (with respect to nSpCas9) side-by-side using 5 pegRNA/ngRNA combinations to target endogenous sites in the human genome.

FIG. 1I. N-terminal and inlaid fusions with full-length and delta RNAse H truncated MMLV-RT pentamutants. Delta RNAse H (dRH) variants of MMLV-RT show comparable or increased prime editing efficiencies at two target sites in human cells, compared to full-length MMLV-RT when fused at the N-terminus of nSpCas9 or inlaid into nSpCas9 between residues G1247/S1248 or G1055/E1056.

FIG. 1J. Different N-terminally fused MMLV-RT variants show similar prime editing efficiencies. Prime editing efficiencies of nSpCas9 (nCas9) negative control, PE3 positive control, PE3 with C-terminal fusion of delta RNAse H variant of MMLV-RT (PE3_dRH). PE3 with combined truncation of 23 N-terminal amino acids and of RNAse H domain (PE3_d23_dRH), N-terminal MMLV-RT full length fusion, and N-terminal fusion of MMLV-RT delta RNAse H (N-terminal MMLV_dRH) in HEK293T cells across 5 endogenous target sites.

FIGS. 1K-N. Additional data comparing intact and split PE variants, including the G1055 inlaid PE variant, SaPE(KKH), and Split-SaPE(KKH). K, Dot and bar plots showing the PPE, IPE, and byproduct or combined IPE and byproduct frequencies for the negative controls of experiments shown in FIGS. 1D-G. FIG. 2B (left of the dashed line), and L of this figure. Controls shown are of a nSpCas9 and a ‘no treatment’ for each of the 11 pegRNA/ngRNA combinations. (n=3; independent replicates). L, Dot and bar plots showing the PPE, IPE, and byproduct or combined IPE and byproduct frequencies for a PE2 fusion variant with MMLV-RT inlaid at position G1055, using 11 peg/ngRNA combinations in HEK293T cells (n=3; independent replicates). Negative controls for this experiment are shown in K. M, Scatter plot based on simple linear regression, comparing prime editing frequencies across 11 tested pegRNA/ngRNA combinations with Split-PE2 and PE2 constructs in HEK293T cells (same data as shown in FIGS. 1D-G). Dashed regression line is superimposed on the scatter plot. r²=1−(SSreg/SStot) and quantifies goodness of fit for the results of linear regression. (n=3; independent replicates). N, Dot and bar plots showing frequencies of PPE and combined IPE and byproduct frequencies in HEK293T cells using six pegRNA/ngRNA combinations and prime editors that use the N580A nickase variant of the Staphylococcus aureus Cas9 (nSaCas9) KKH PAM recognition variant for both a C-terminal fusion of MMLV-RT mutant and a Split-PE configuration. The data are shown alongside nSaCas9(KKH) and no treatment controls. All targeted sites harbor NNGRRT protospacer adjacent motif (PAM) sequences, and all prime edits are CTT insertions. (n=3; independent replicates).

FIGS. 1O-P. Activities of intact and split MMLV-RT and Marathon-RT based PE architectures in U2OS cells and in human iPSC-derived cardiomyocytes (hiPSC-CMs). O, Dot and bar plots showing PPE, IPE and byproduct frequencies or combined IPE and byproducts induced by MMLV-RT-ΔRH and Marathon-RT based PEs as well as controls using 8 peg/ngRNA combinations in U2OS cells. (n=3; independent replicates) P, Dot and bar plots showing PPE, IPE and byproduct frequencies or combined IPE and byproducts induced by PE2-ΔRH, Split-PE2-ΔRH and a control using 4 peg/ngRNA combinations in hiPSC-derived cardiomyocytes (Fujifilm iCell Cardiomyocytes). (n=3; independent replicates).

FIG. 1Q. Assessment of Cas9 and/or pegRNA-dependent off-target editing activities of Split-PE2 compared with PE2. Heatmaps showing editing frequencies of PE2, Split-PE2, and a negative control. Editing is represented in color gradients from light grey to darker grey (see keys on the right of each heatmap). Darker shading indicates relevant prime editing (on-target) or indel frequencies (off-target). Frequencies are also shown numerically per replicate. Genomic loci are indicated above each heatmap. The desired on-target editing outcome is indicated in the first row. Editing frequencies are shown for single replicates. Off-target site labels are colored in grey. (n=3; independent replicates).

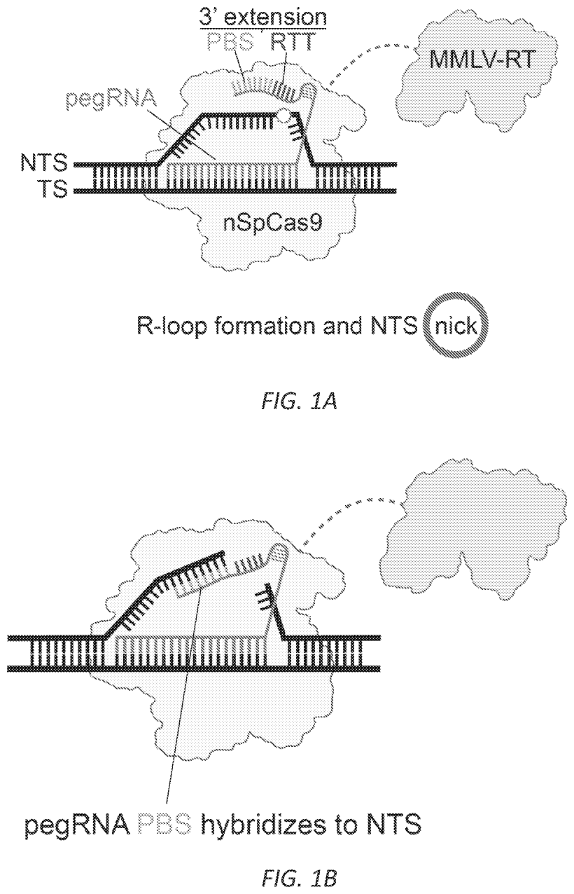

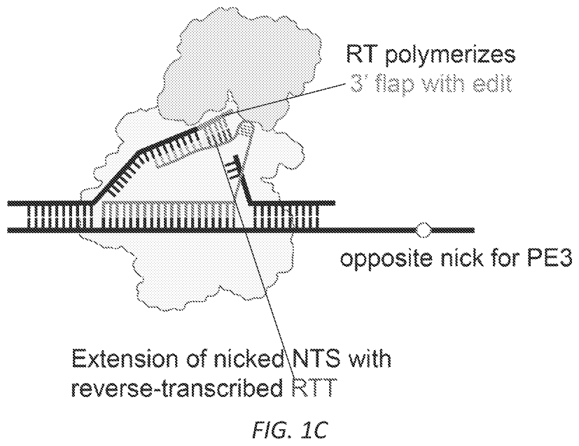

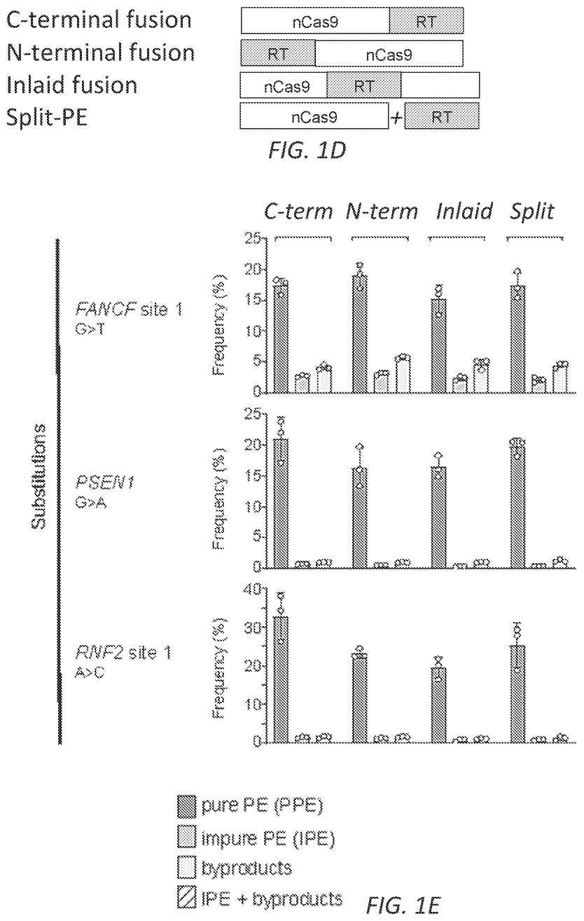

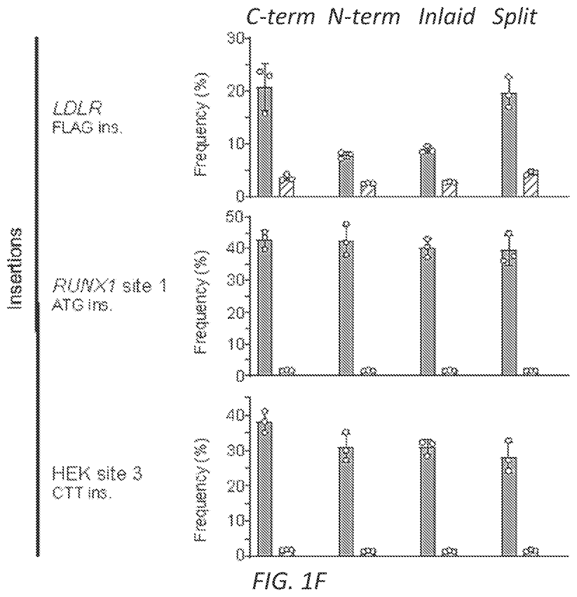

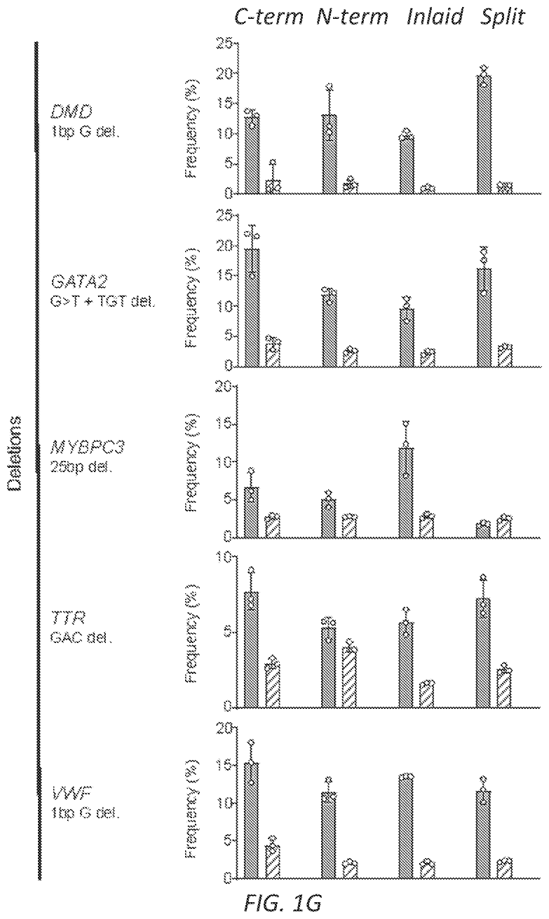

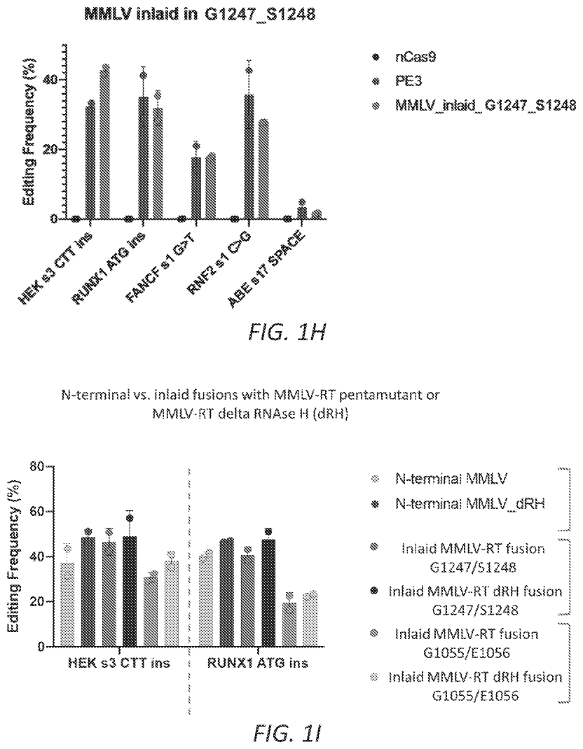

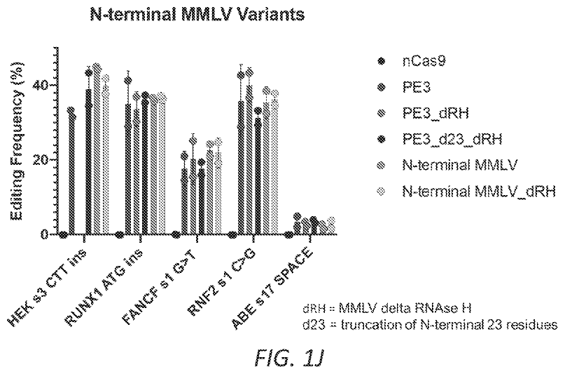

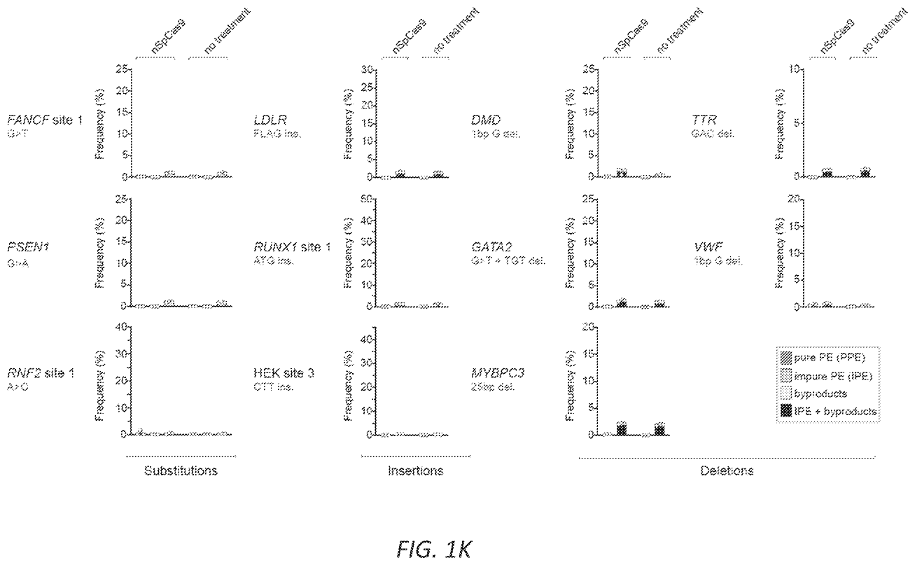

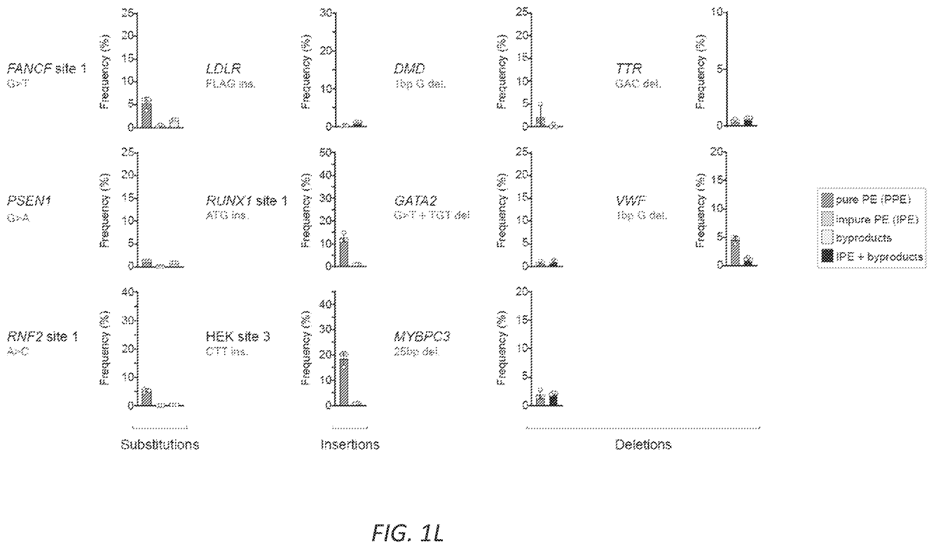

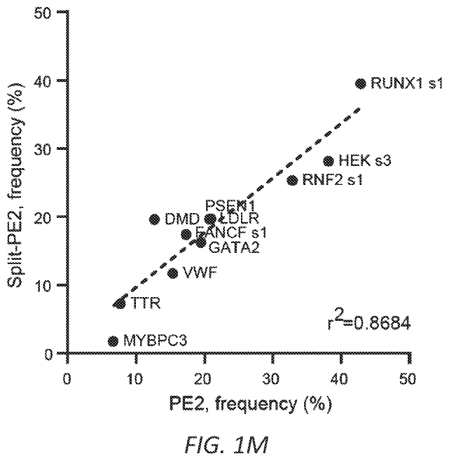

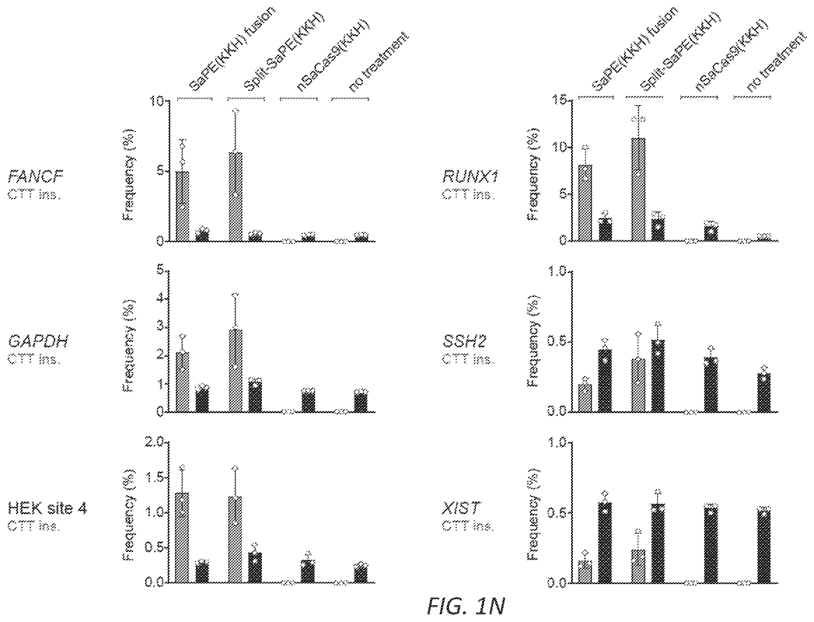

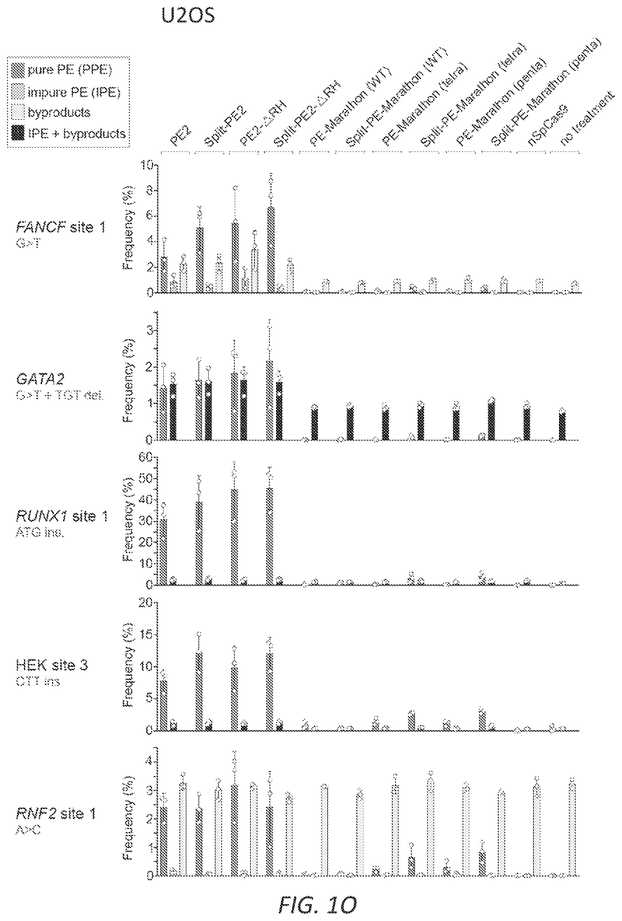

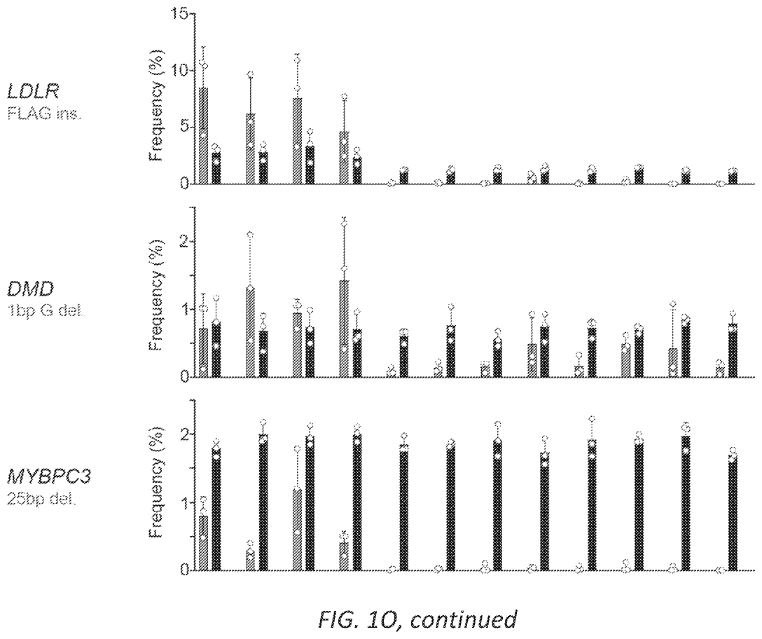

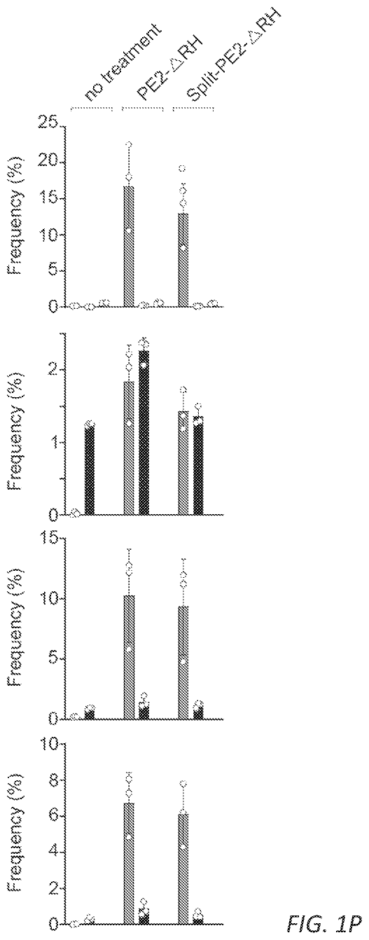

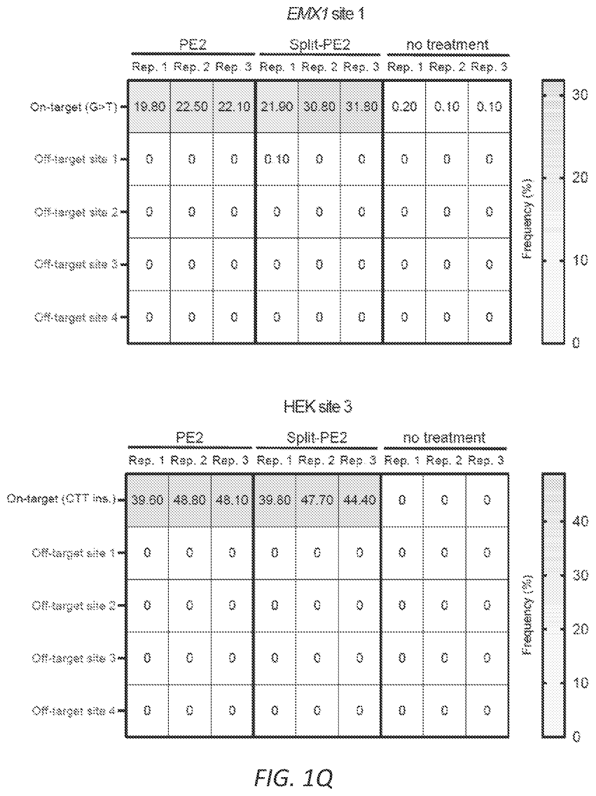

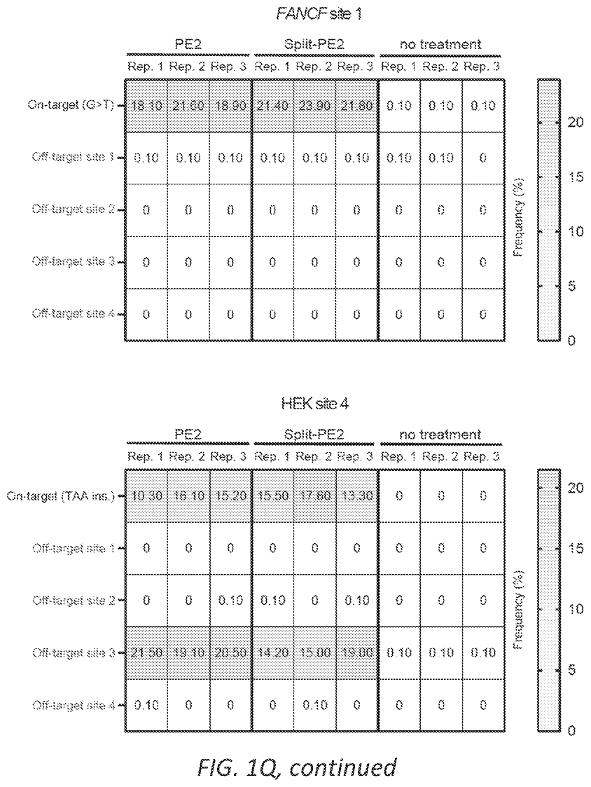

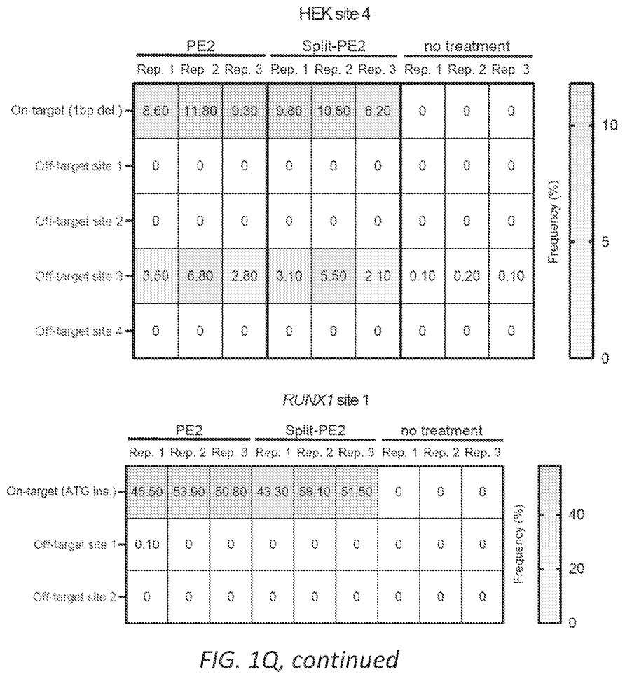

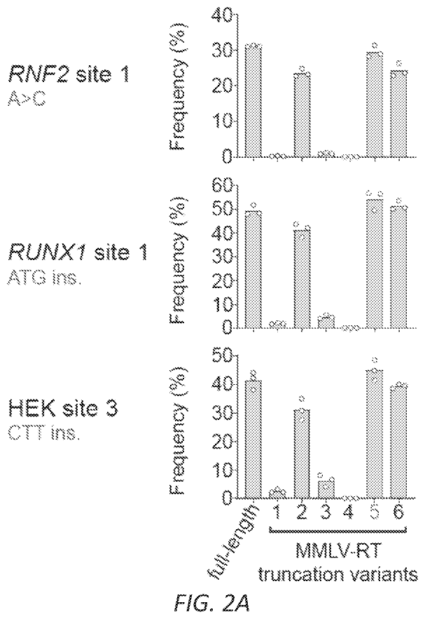

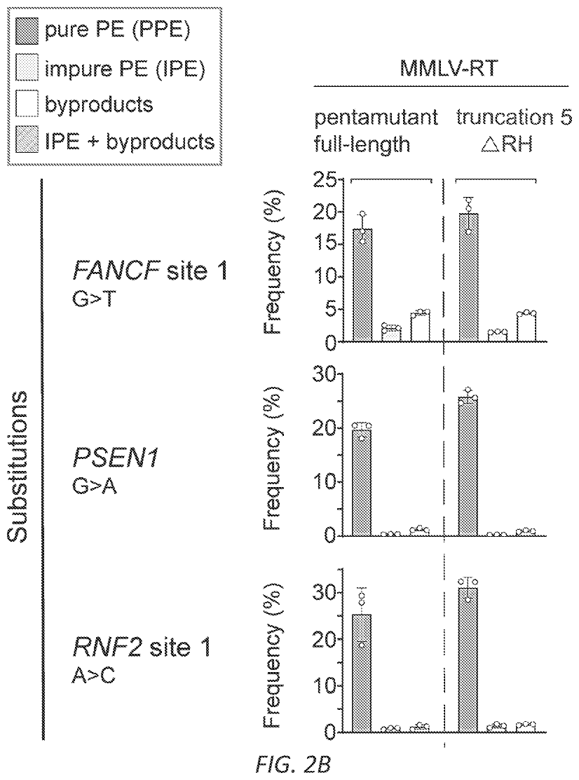

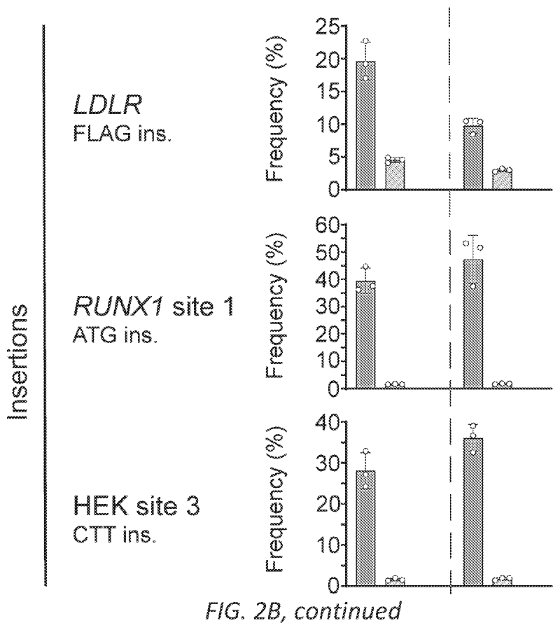

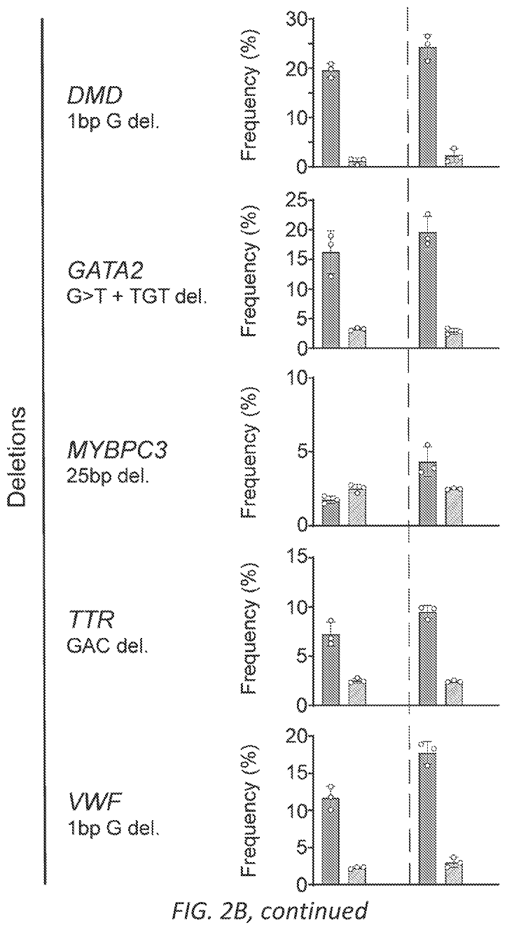

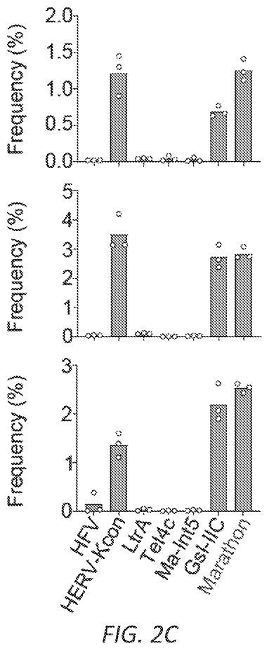

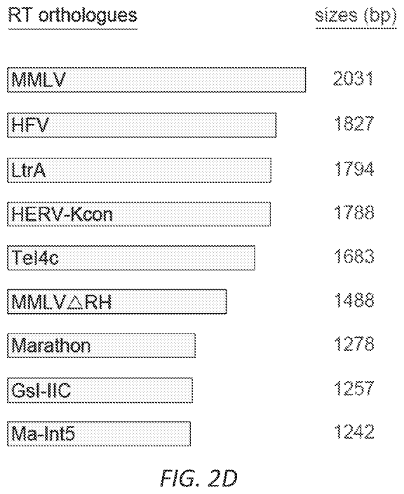

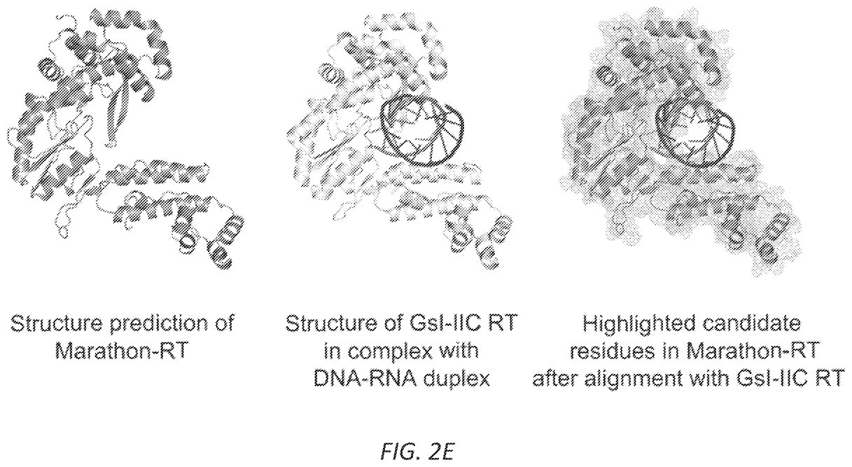

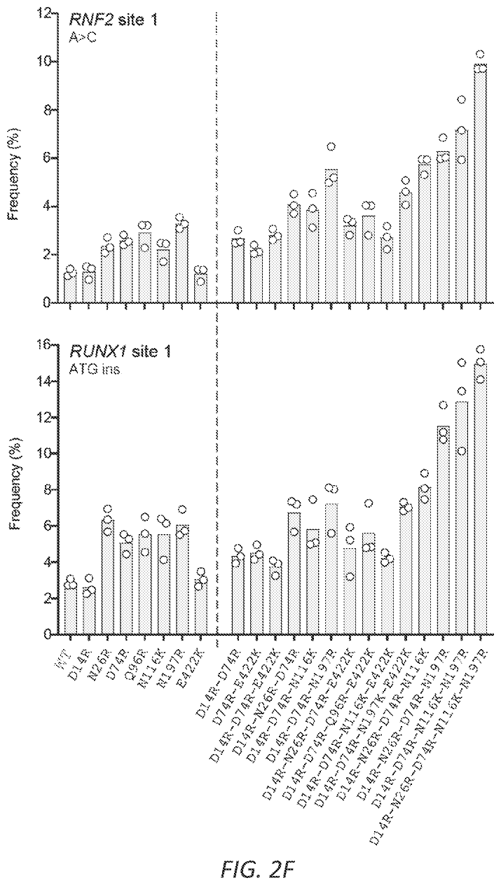

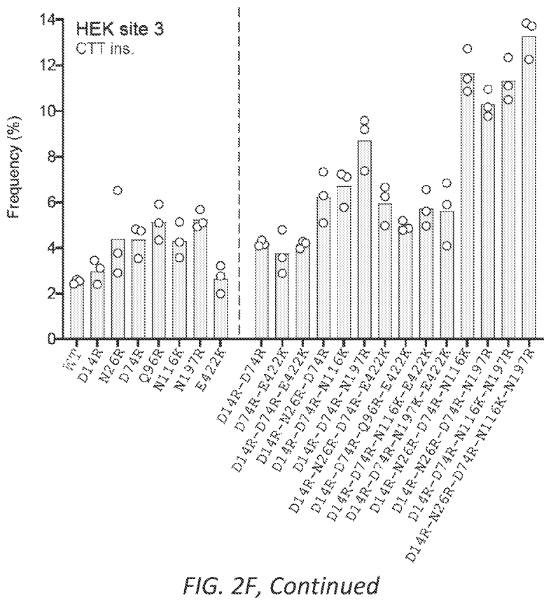

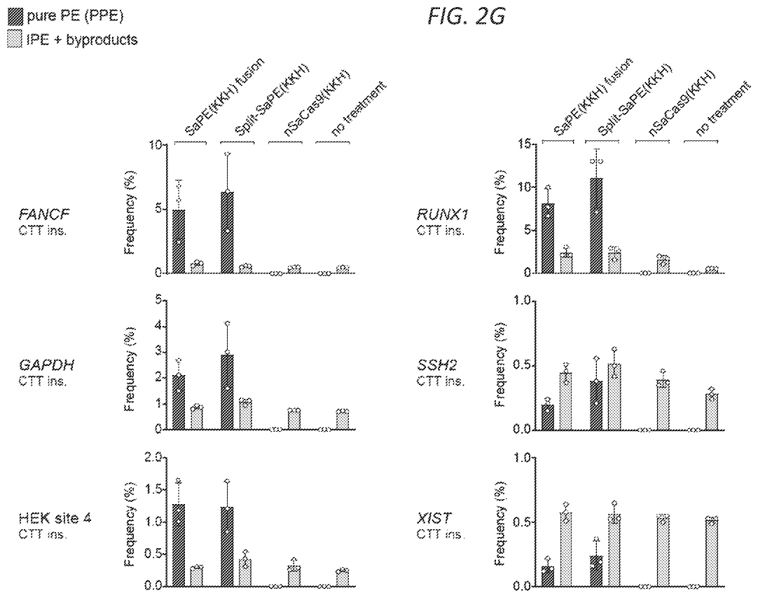

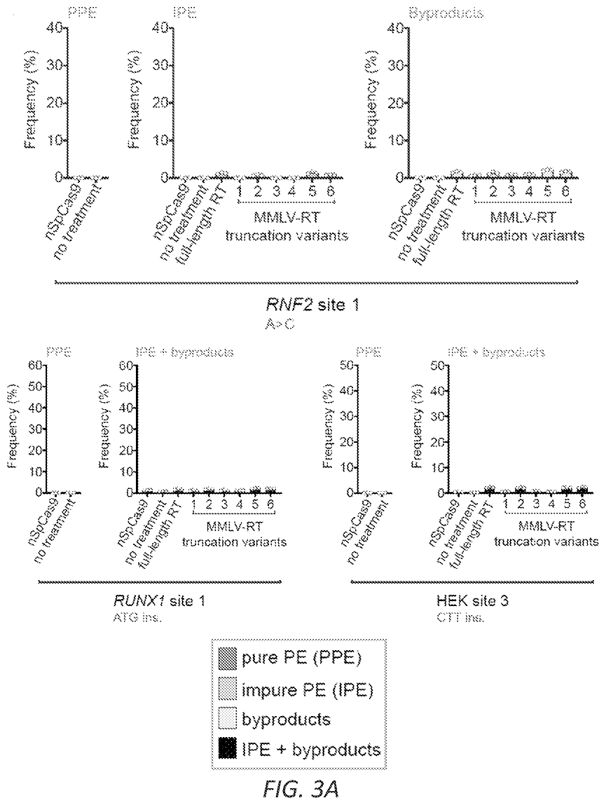

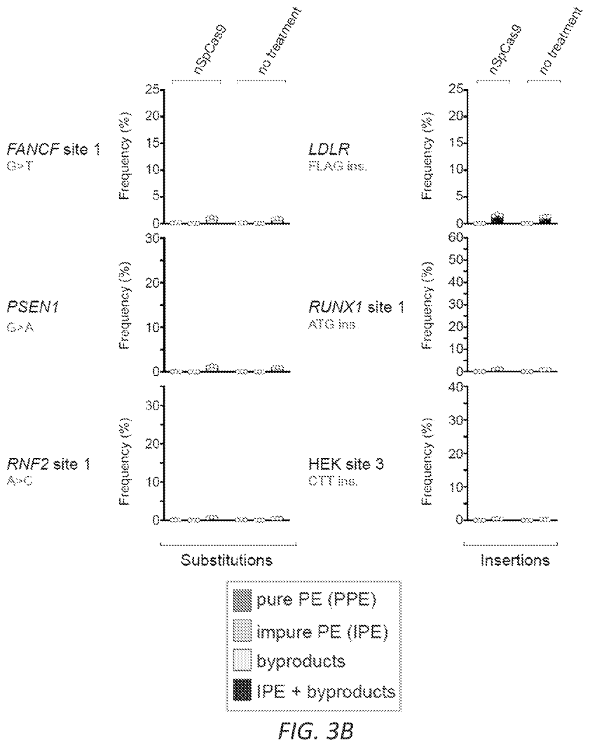

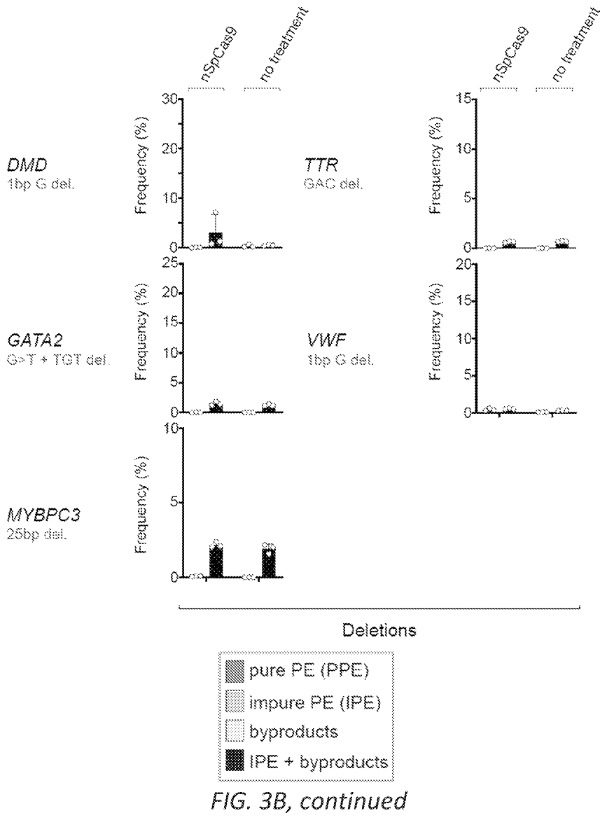

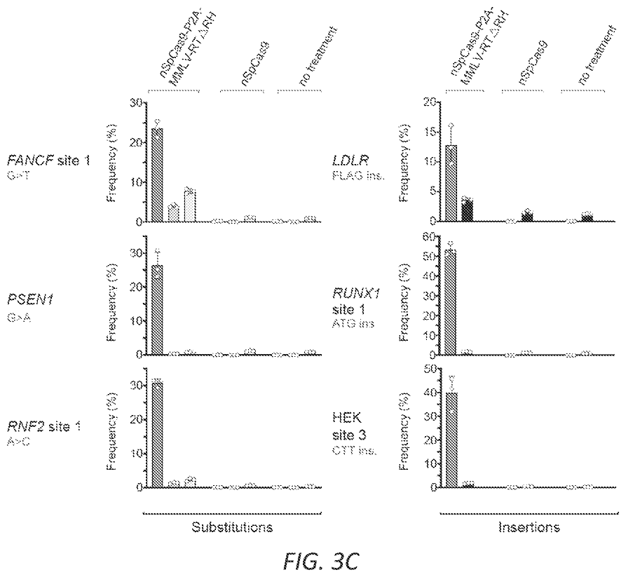

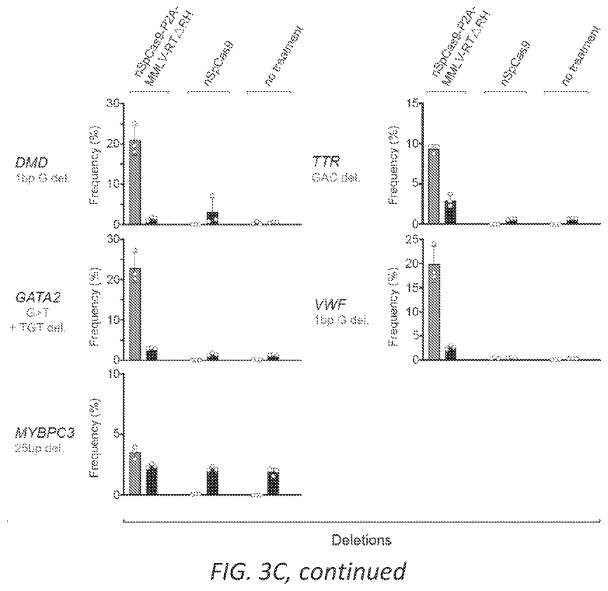

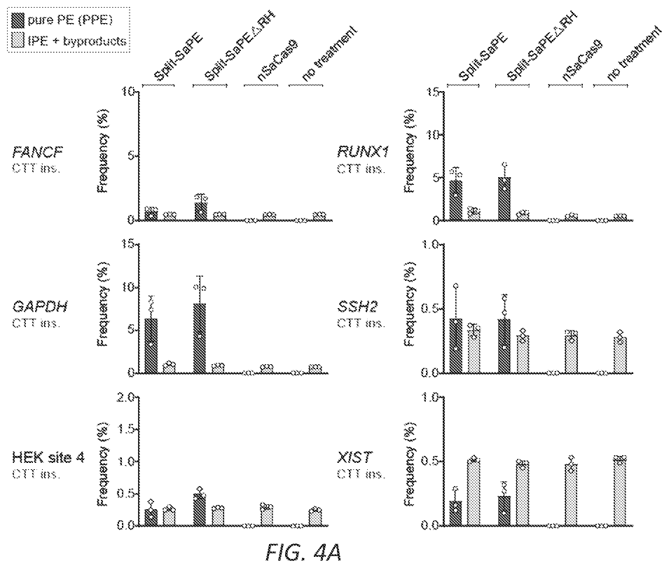

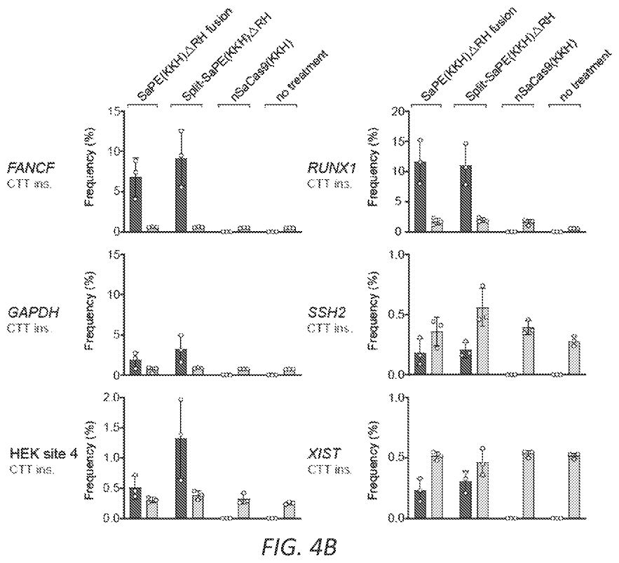

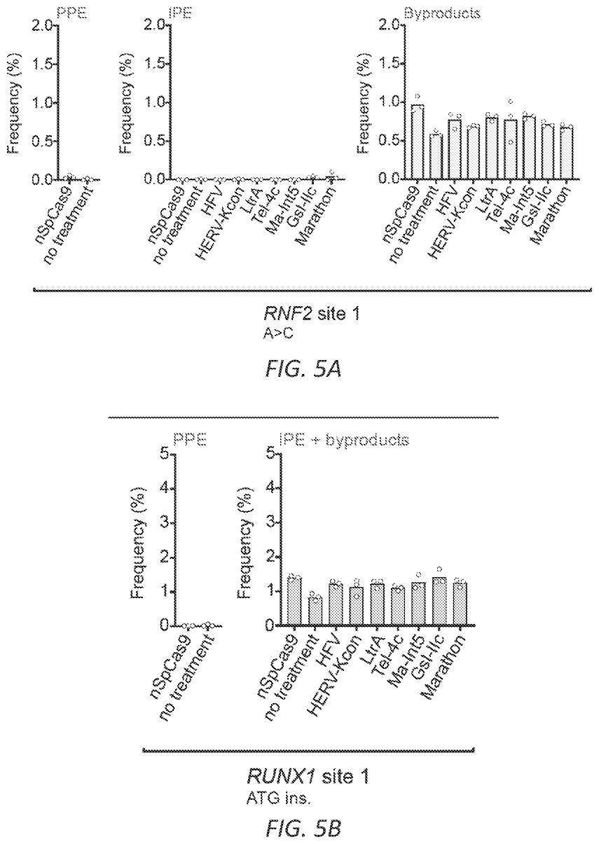

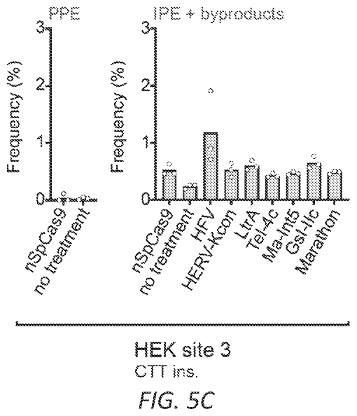

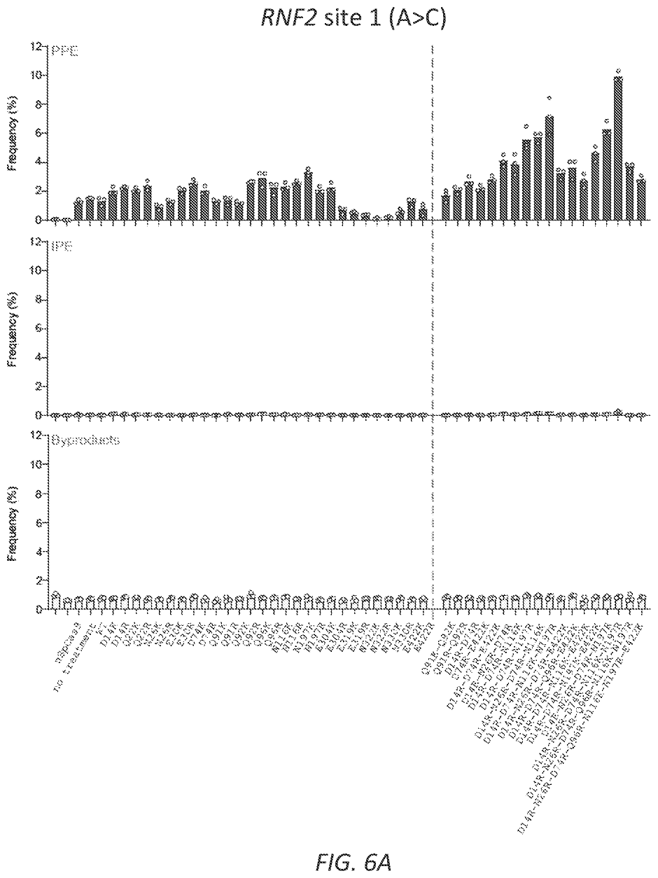

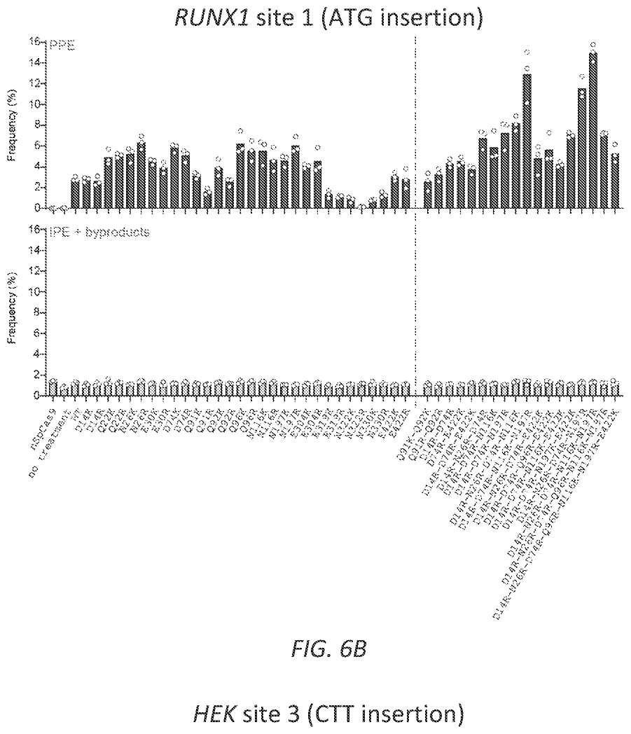

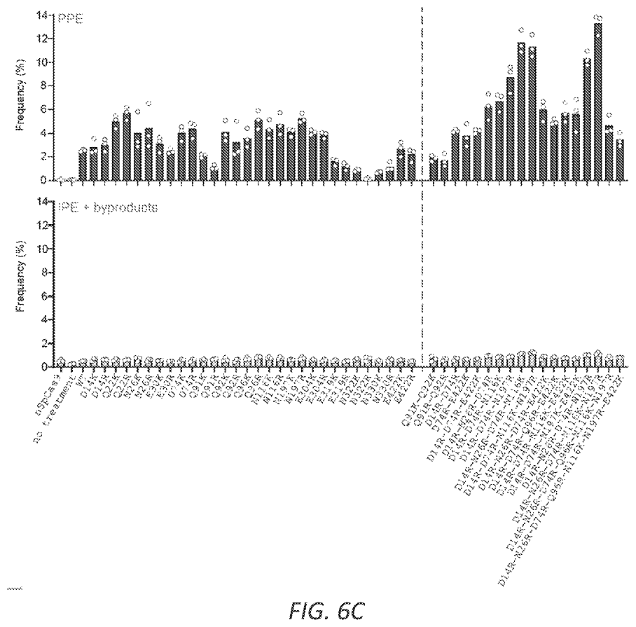

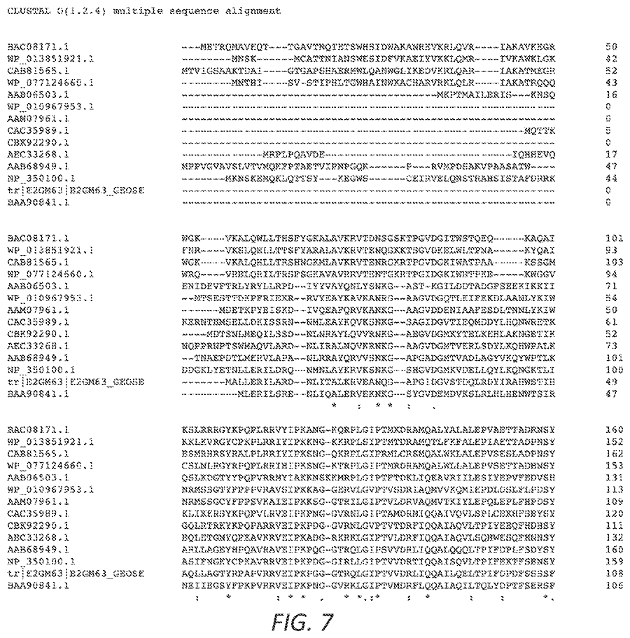

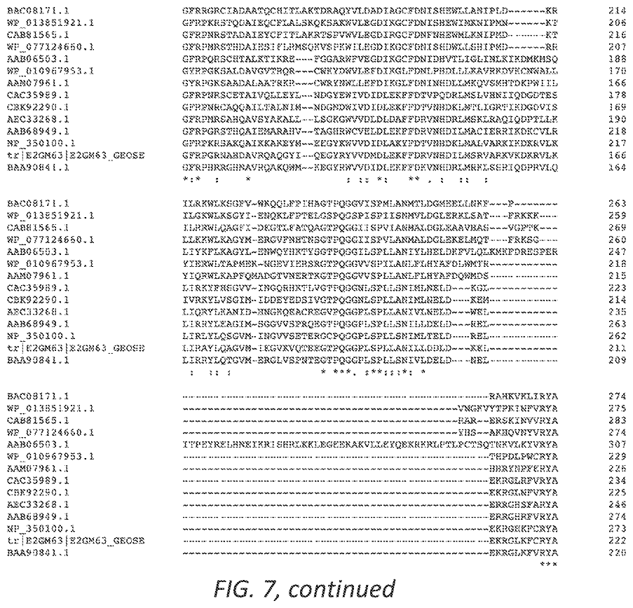

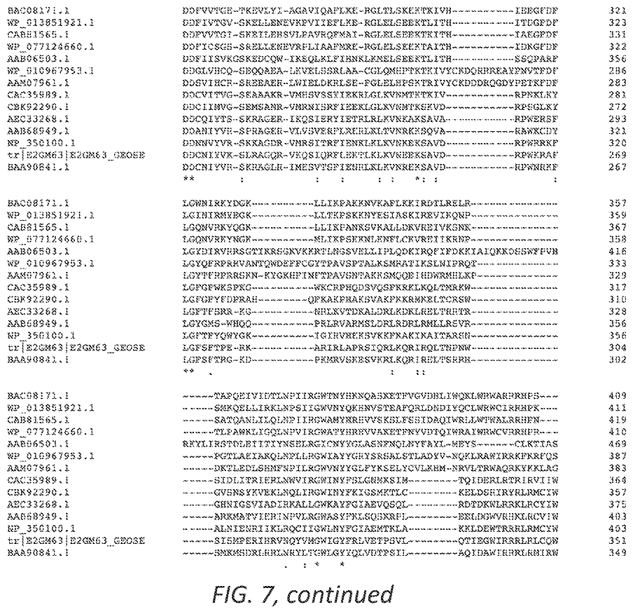

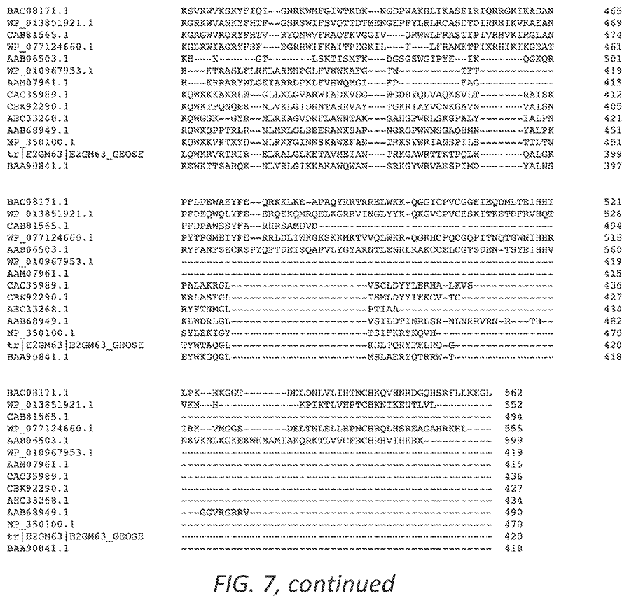

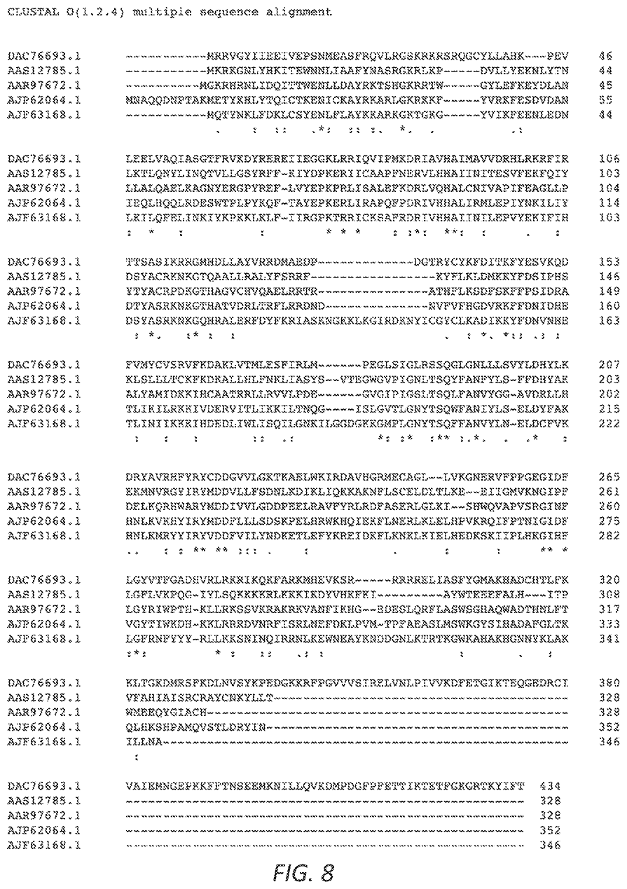

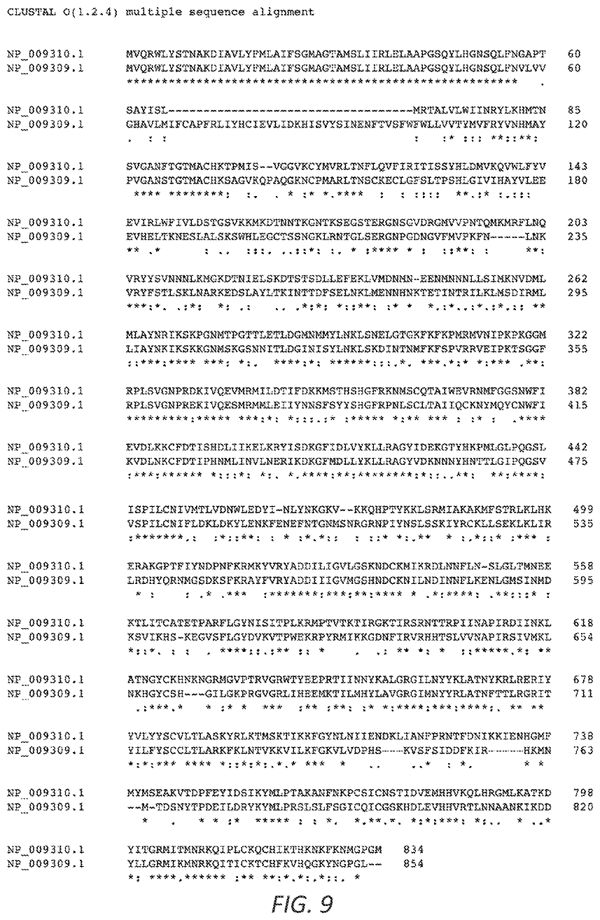

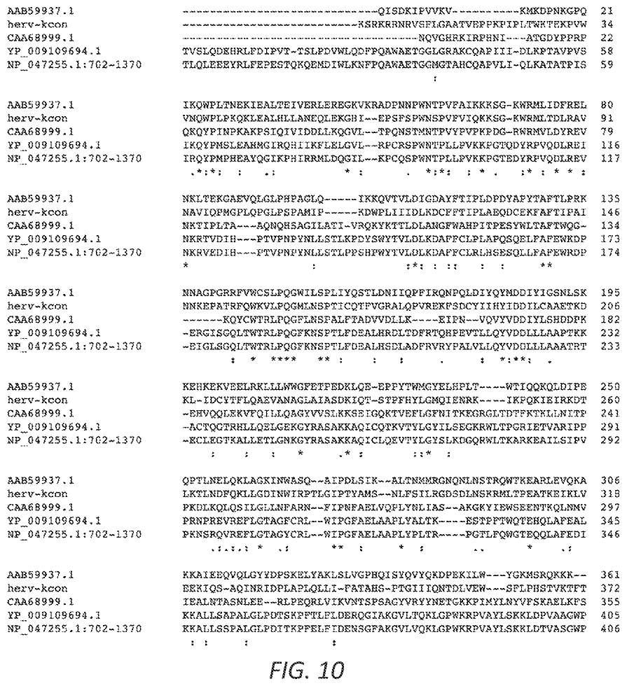

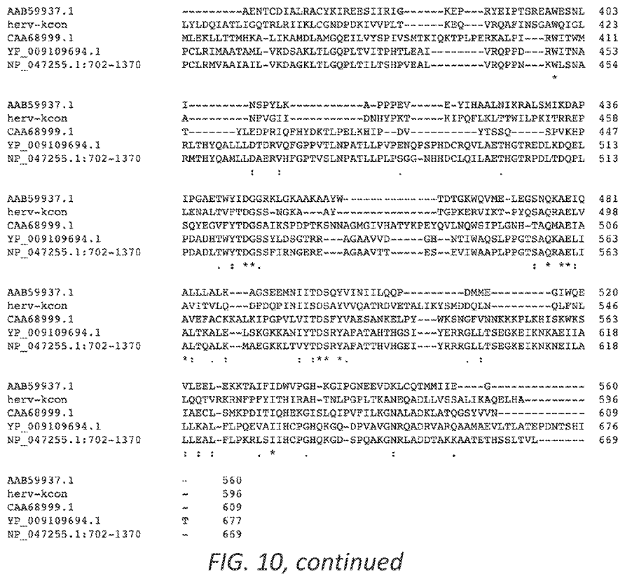

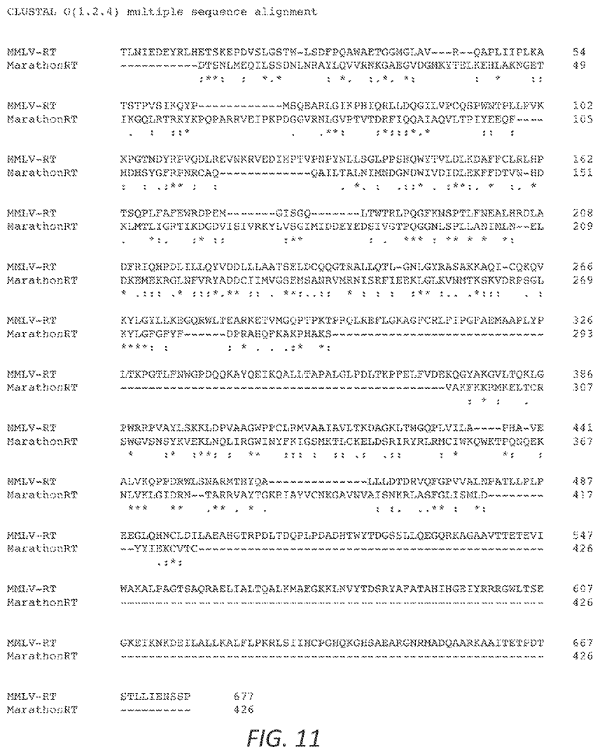

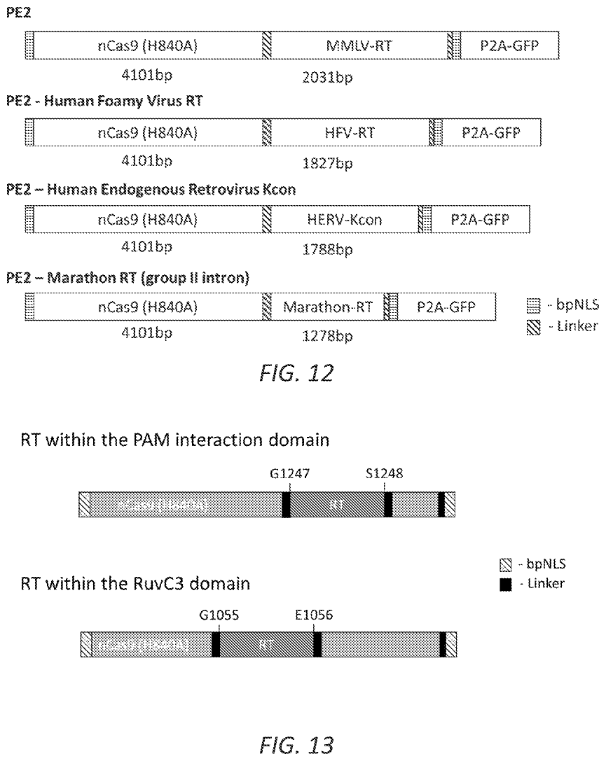

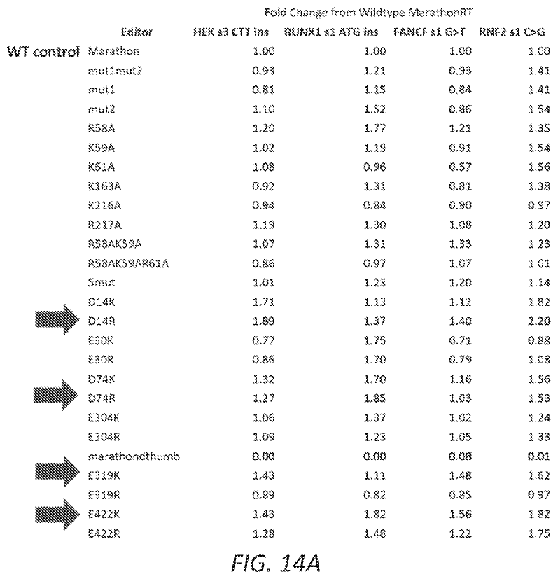

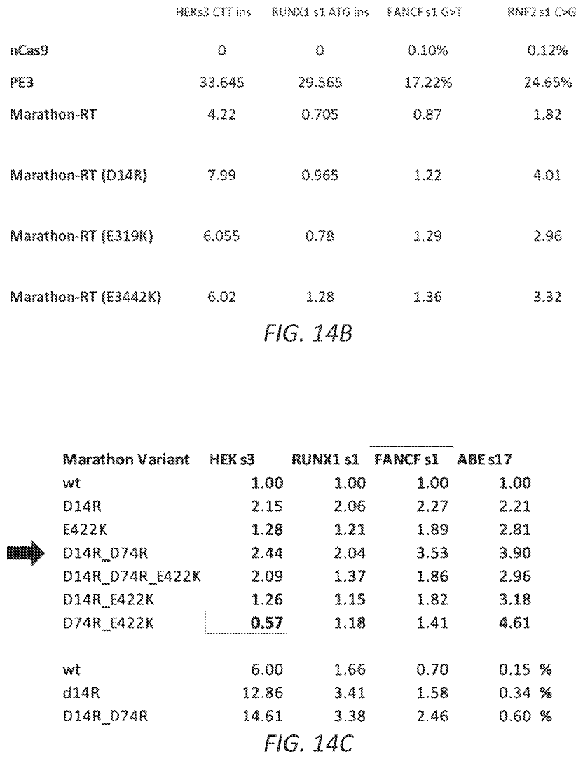

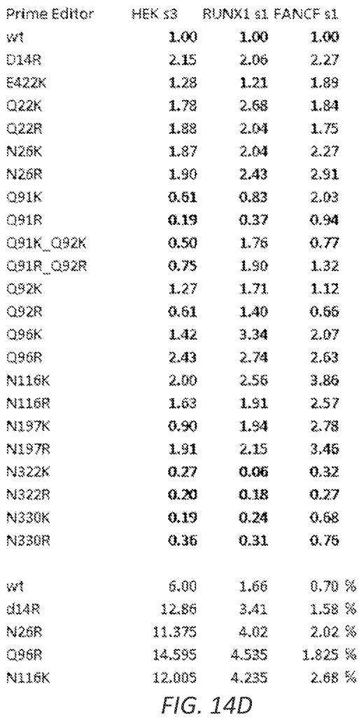

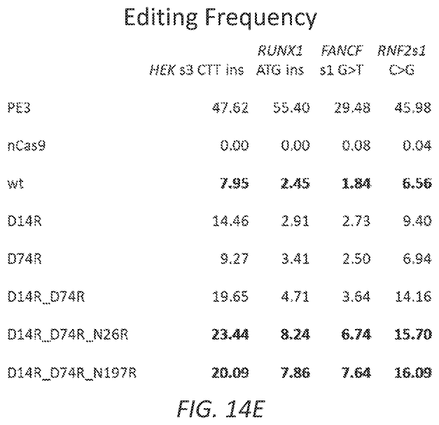

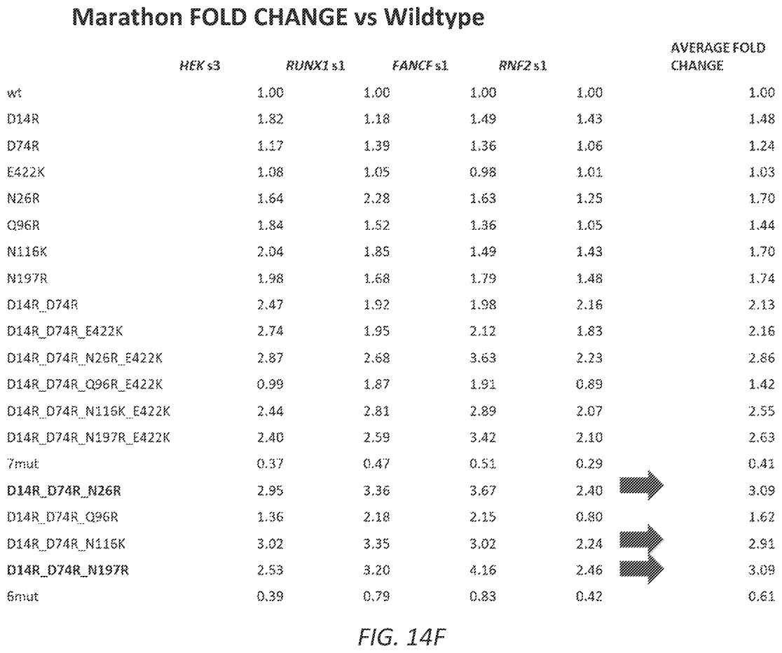

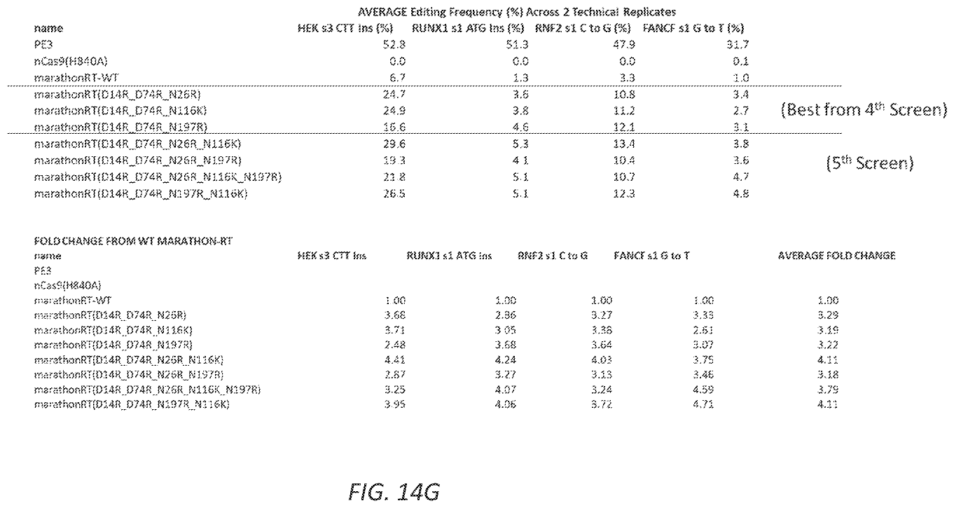

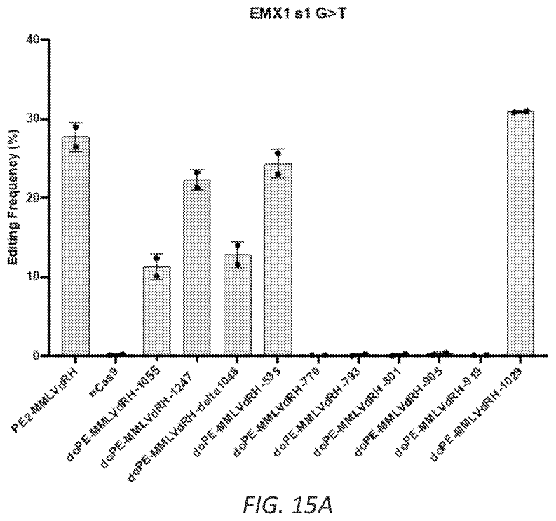

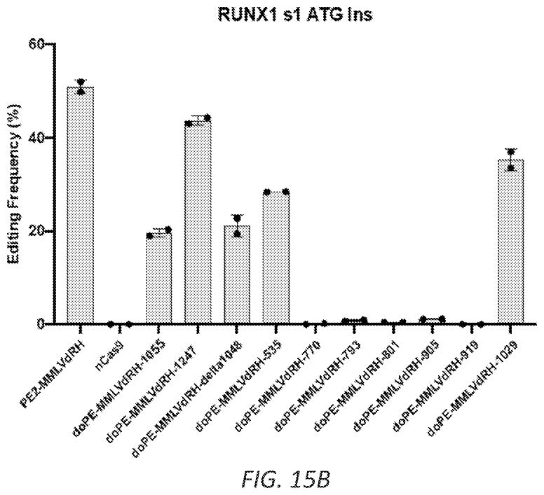

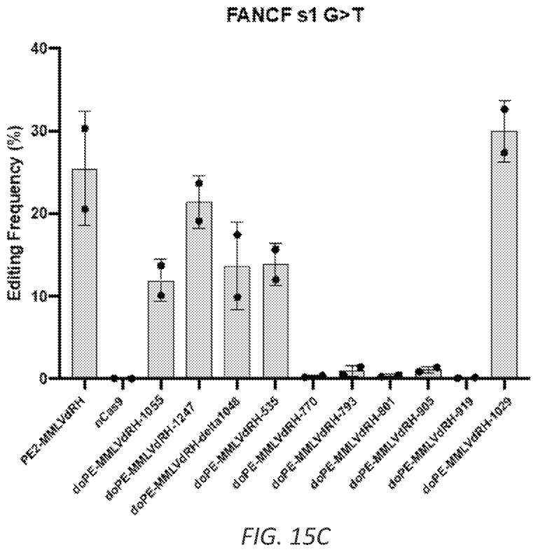

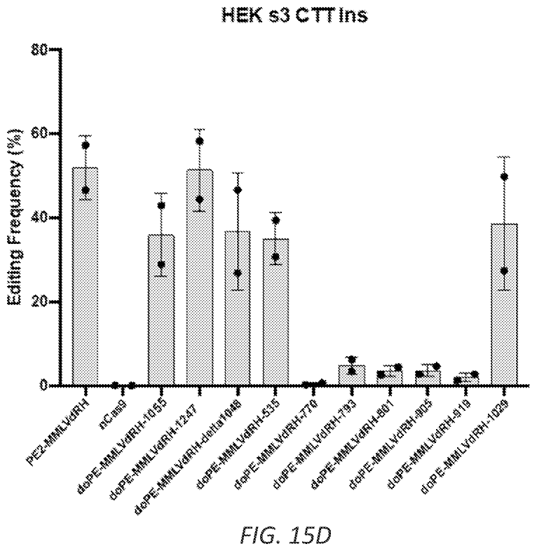

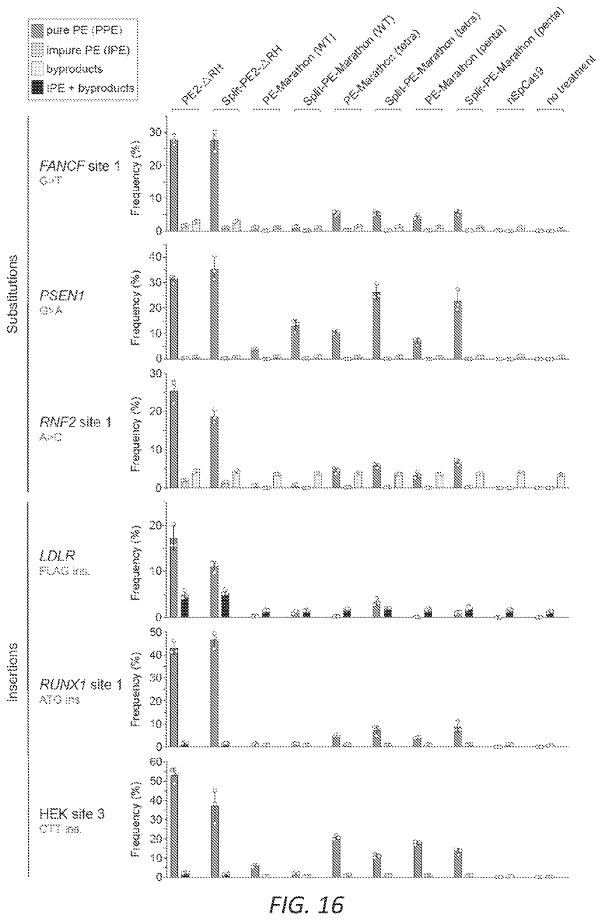

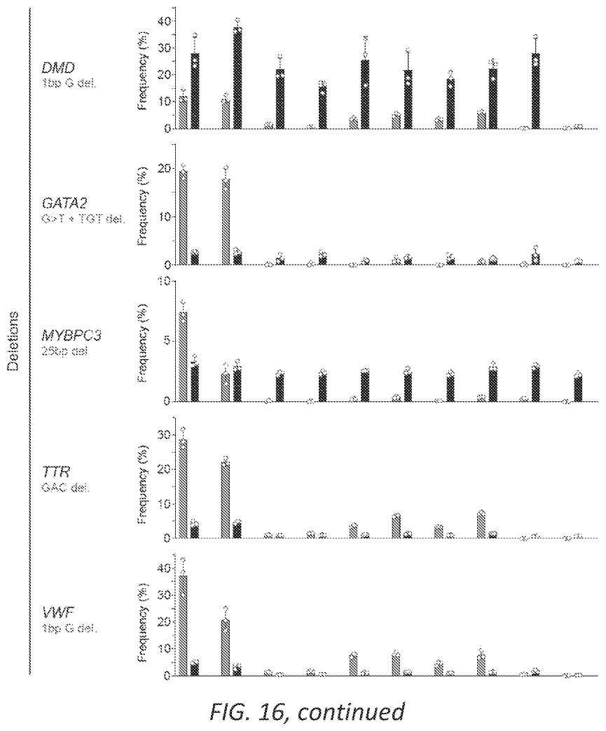

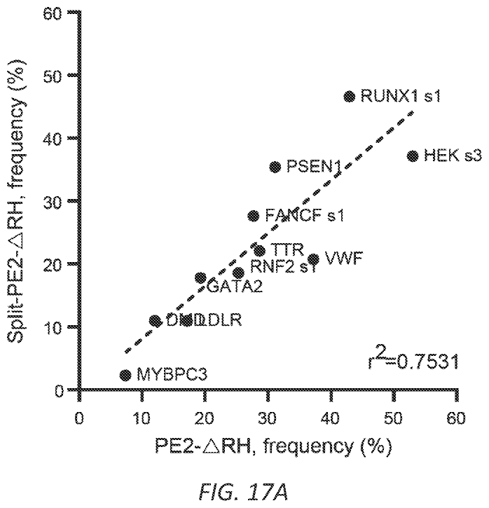

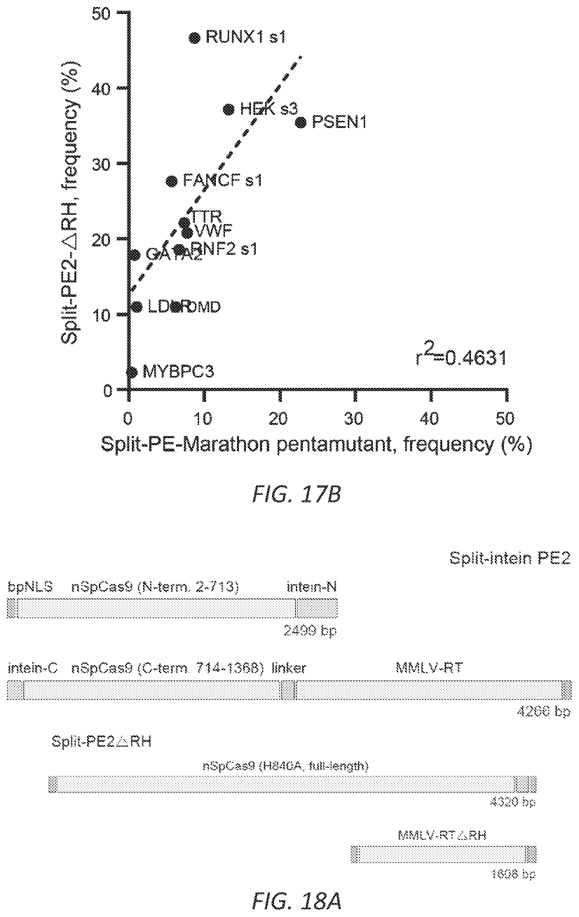

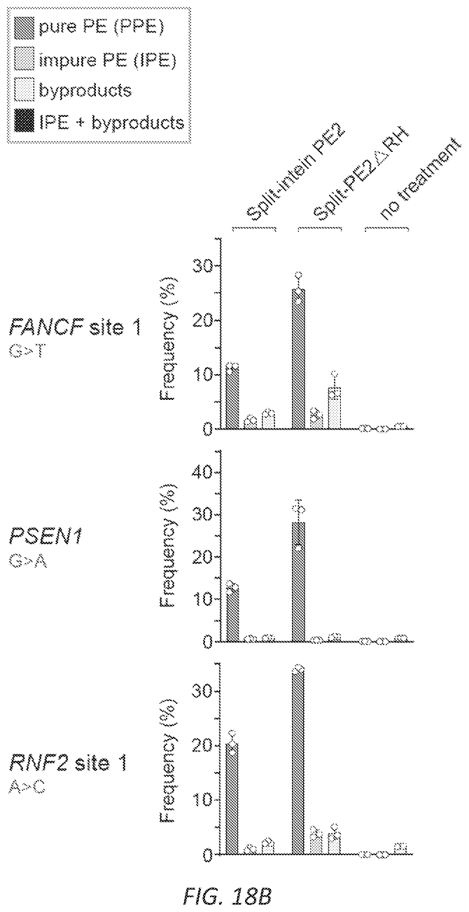

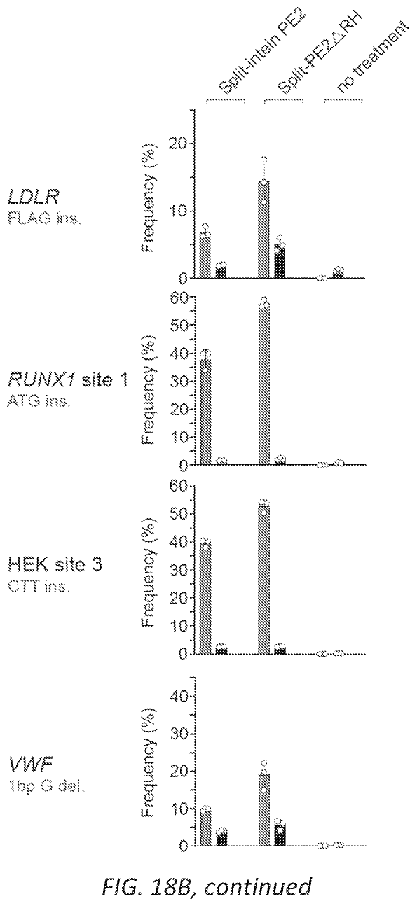

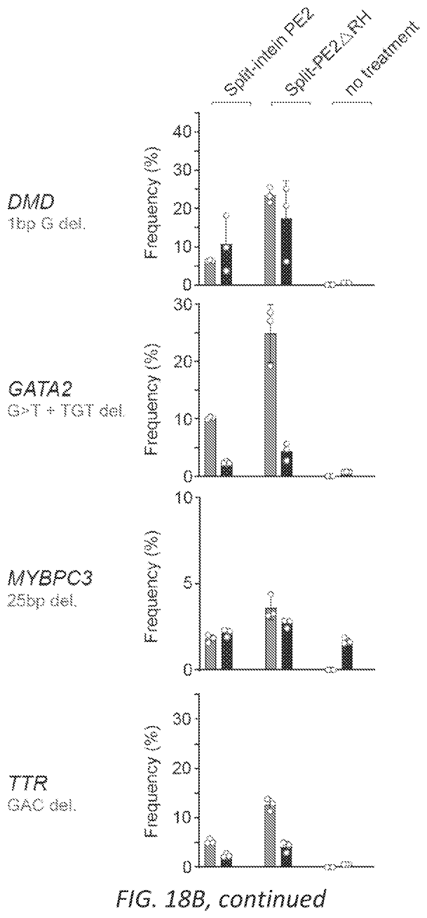

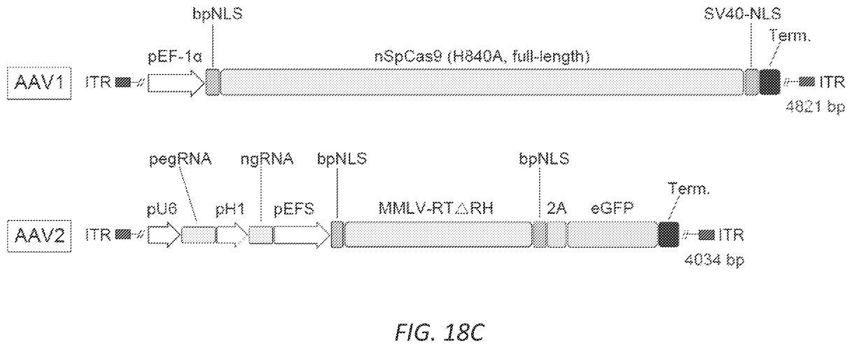

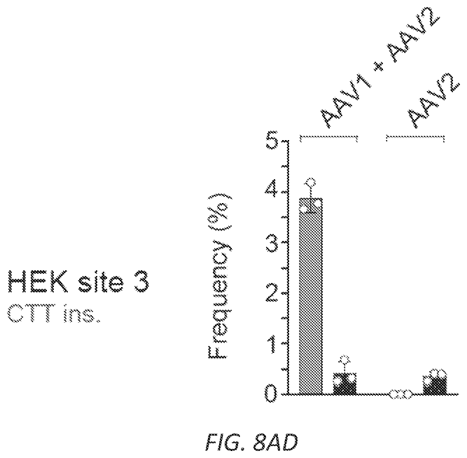

FIGS. 2A-G. Rapid screening of variant RT domains using the Split-PE platform. A, Dot and bar plots showing PPE frequencies induced by co-expression of nSpCas9 and full-length Moloney Murine Leukemia Virus Reverse Transcriptase (MMLV-RT) pentamutant or each of six truncation variants thereof tested with three different pegRNA/ngRNA combinations in HEK293T cells (ΔRH variant highlighted in pink). Experiments were performed as technical replicates and so no error bars are shown (also applies to C and F). n=3, technical replicates. B, Dot and bar plots comparing PPE, IPE, and byproduct or combined IPE and byproduct frequencies observed with co-expression of nSpCas9 and the MMLV-RT truncation 5 (ΔRH) or the full-length MMLV-RT pentamutant together with 11 pegRNA/ngRNA combinations in HEK293T cells. Data shown for full-length MMLV-RT (left of the dashed line) are the same as those shown for Split-PE in FIGS. 1E-G (n=3; independent replicates). C, Dot and bar plots showing PPE frequencies of seven non-MMLV RTs tested with nSpCas9 and three pegRNA/ngRNA combinations in HEK293T cells. Non-MMLV RTs tested were from human foamy virus (HFV), human endogenous retrovirus K (HERV-Koon; derived consensus sequence), lactococcal group II intron L1.ltrB (LtrA), Thermosynechococcus elongatus group II intron (TeI4c), Methanosarcina aromaticovorans intron 5 (Ma-Int5), Geobacillus stearothermophilus GsI-IIC intron (GsI-IIC), and Eubacterium rectale (Eu.re.I2) group II intron (Marathon). n=3, technical replicates D, Schematic showing the lengths of all non-MMLV RTs tested in c in comparison to MMLV-RT. E, Structural representation (cartoon) of Marathon-RT (left, based on a Phyre2 structure prediction) and GsI-IIC RT (middle) in complex with an RNA template-DNA primer duplex (PDB accession 6AR1), and Marathon-RT (right cartoon) with highlighted candidate residues that are located within the modeled DNA/RNA binding pocket, based on the alignment with GsI-IIC. All graphical representations were generated with PyMol (Methods). F, Dot and bar plots showing the PPE frequencies of the seven Marathon-RT single residue mutants (left of dashed line) that were used to generate the 14 most efficient Marathon-RT combination variants (right of dashed line), both in HEK293T cells. The data for wild-type (WT) Marathon-RT pentamutant shown are the same as those shown in C. n=3, technical replicates. PPE frequencies induced by all 30 single and 18 combinatorial variants (inclusive of those shown here) are presented in FIG. 6. G, Dot and bar plots showing frequencies of PPE and combined IPE and byproduct frequencies in HEK293T cells using six pegRNA/ngRNA combinations and prime editors that use the N580A nickase variant of the Staphylococcus aureus Cas9 (nSaCas9) KKH PAM recognition variant for both a C-terminal fusion of MMLV-RT mutant and a Split-PE configuration. The data are shown alongside nSaCas9(KKH) and no treatment controls. All targeted sites harbor NNGRRT protospacer adjacent motif (PAM) sequences, and all prime edits are CTT insertions. (n=3; independent replicates).
Full length WT/pentamutant=677AA
Truncation 1: 431AA, delta 432-677
Truncation 2: 654 AA, delta 1-23
Truncation 3: 470AA, delta 471-677
Truncation 4: 361AA, delta 362-677
Truncation 5: 496AA, delta 497-677
Truncation 6: 473AA, delta 1-23+497-677

FIGS. 3A-C. Additional data from experiments assessing activities of MMLV-RT truncations and co-translationally expressed Split-PE with the MMLV-RTΔRH variant. A, Dot and bar plots showing PPE, IPE and byproduct frequencies or combined IPE and byproducts for the negative controls of the experiments shown in FIG. 2A as well as IPE and byproducts or combined IPE and byproducts for the truncation variants shown in FIG. 2A. Experiments were performed as technical replicates and so no error bars are shown (n=3; technical replicates). B, Dot and bar plots showing PPE, IPE and byproduct frequencies or combined IPE and byproducts for the negative controls of the experiments shown in FIG. 2B (right of the dashed line). (n=3; independent replicates). C, Dot and bar plots showing PPE, IPE and byproduct frequencies or combined IPE and byproducts for co-translationally expressed nSpCas9 and MMLV-RTΔRH and negative controls in HEK293T cells. Negative control data are the same as shown in B. (n=3; independent replicates).

FIG. 4. Activities of nSaCas9-based Split-PE architectures with full-length MMLV-RT and MMLV-RTΔRH in HEK293T cells. A, Dot and bar plots showing the frequencies of PPE and combined IPE and byproducts in HEK293T cells induced by nSaCas9 co-expressed with either full-length MMLV-RT (Split-SaPE) or MMLV-RTΔRH (Split-SaPEΔRH) and six pegRNA/ngRNA combinations. Negative control “no treatment” data are the same as shown in FIGS. 2G and 4B). (n=3; independent replicates). B, Dot and bar plots showing the frequencies of PPE and combined IPE and byproducts in HEK293T cells induced by either a fusion of nSaCas9-KKH(N580A) to MMLV-RTΔRH (SaPE(KKH)ΔRH fusion) or a Split-PE setup with co-expression of nSaCas9-KKH(N580A) and MMLV-RTΔRH (Split-SaPE(KKH)ΔRH) using six pegRNA/ngRNA combinations. The nSaCas9-KKH(N580A) and no treatment negative controls are the same as shown in FIGS. 2G and 4A. (n=3; independent replicates).

FIGS. 5A-C. Additional data from experiments assessing activities of Split-PEs with non-MMLV RTs. Dot and bar plots showing PPE frequencies from negative controls and IPE and byproduct or combined IPE and byproduct frequencies for the negative controls (same as shown in FIGS. 3A and 6) and different RTs tested in the experiments that correspond to FIG. 2C, using three peg/ngRNA combinations in HEK293T cells. A, RNF2 site 1 (A>C); B. RUNX1 site 1 (ATG insertion); C, HEK site 3 (CTT insertion). (n=3; technical replicates).

FIGS. 6A-C. Additional data from the Marathon-RT engineering experiment. Dot and bar plots showing PPE, IPE and byproduct frequencies or combined IPE and byproducts induced in negative controls (same as shown in FIGS. 3A and 5A-C) and by all Marathon-RT single and combinatorial mutation variants we screened using three peg/ngRNA combinations in HEK293T cells. Data for the subset of variants (and WT Marathon-RT) shown in FIG. 2F are the same as those shown here. Variants shown to the left of the dashed line are single mutation variants while those to the right of the line are combinatorial mutation variants. A, RNF2 site 1 (A>C); B, RUNX1 site 1 (ATG insertion); C, HEK site 3 (CTT insertion).

FIG. 7. Amino acid sequence alignment of 14 group II intron reverse transcriptases from Table B. Alignments were performed using the Clustal Omega multiple sequence alignment tool. Shown are SEQ ID NOs. 121-134.

FIG. 8. Amino acid sequence alignment of 5 diversity generating retroelement reverse transcriptases from Table B. Alignments were performed using the Clustal Omega multiple sequence alignment tool. Shown are SEQ ID NOs. 150-154.

FIG. 9. Amino acid sequence alignment of 2 yeast group II intron reverse transcriptases from Table B. Alignments were performed using the Clustal Omega multiple sequence alignment tool. Shown are SEQ ID NOs. 155-156.

FIG. 10. Amino acid sequence alignment of 5 retroviral reverse transcriptases from Table B. Alignments were performed using the Clustal Omega multiple sequence alignment tool. Shown are SEQ ID NOs. 157-161.

FIG. 11. Amino acid sequence alignment of MMLV and Marathon reverse transcriptases from Table B. Alignments were performed using the Clustal Omega multiple sequence alignment tool. Shown are SEQ ID NOs. 162-163.

FIG. 12. Prime Editor alternative RT fusions.

FIG. 13. Schematic illustrations of exemplary inlaid constructs.

FIGS. 14A-G. Fusion Prime Editors with MarathonRT (WT) and Marathon-RT variants. A and B, activity of single mutants. C, Combined Variants—Fold change from wildtype Marathon-RT. D-G, Marathon-PE variants (fusion), with mutations of long, neutral amino acids glutamine (Q) and asparagine (N) to charged amino acids Lysine (L) and arginine (R) as well as combinatorial variants thereof with two to seven combined residue changes. 6 mut=D14R_D74R_N26R_Q96R_N116K_N197R; 7 mut=D14R_D74R_N26R_Q96R_N116K_N197R_E422K: D shows fold change on top and editing frequency on the bottom, E shows editing frequency only, F shows fold change only. G shows editing frequency and fold change.

FIGS. 15A-D. Inlaid Prime Editors with truncated MMLV RT (delta RNAse H, truncation 5). Shown is the on-target editing frequency of indicated mutants at EMX1 site 1 (A); RUNX1 site 1 (B); FANCF site 1 (C); and HEK site 3 (D).

FIG. 16. Activities of intact and split size-reduced PE architectures in HEK293T cells. Dot and bar plots showing PPE. IPE and byproduct frequencies or combined IPE and byproducts induced by MMLV-RT-ΔRH and Marathon-RT based PEs as well as controls using 1l peg/ngRNA combinations in HEK293T cells. (n=3; independent replicates).

FIGS. 17A-B. Scatter plots comparing editing frequencies of different intact and split PE architectures. A, Scatter plot comparing prime editing frequencies across 11 tested pegRNA/ngRNA combinations with Split-PE2-ΔRH and PE2-ΔRH constructs in HEK293T cells (same data as shown in FIG. 16). Dashed line shown was determined using simple linear regression. (n=3; independent replicates) B, Scatter plot based comparing prime editing frequencies across 11 tested pegRNA/ngRNA combinations with Split-PE2-ΔRH and Split-PE-Marathon (pentamutant) constructs in HEK293T cells (same data as shown in FIG. 16). Dashed line shown was determined using simple linear regression. (n=3; independent replicates).

FIGS. 18A-D. Comparison of Split-PEΔRH with a split-intein PE system in HEK293T cells and dual AAV delivery of Split-PEΔRH to U2OS cells. A, Schematic of Split-intein PE2 and Split-PE2ΔRH architectures, based on the nSpCas9-H840A variant and MMLV-RT. Both components of both systems were expressed from a CMV promoter. PegRNA and ngRNA plasmids were co-transfected separately and both gRNAs were expressed from a human U6 promoter. Numbers indicate the length of the respective component in base pairs (bp). B, Dot and bar plots showing PPE, IPE and byproduct frequencies or combined IPE and byproducts induced by Split-intein PE2 and Split-PE2ΔRH as well as a no treatment control using 11 peg/ngRNA combinations in HEK293T cells. (n=3; independent replicates). C, Schematic of the Split-PE2ΔRH architecture for dual AAV delivery. D, Dot plot showing PPE and combined IPE and byproducts induced at HEK site 3 (desired edit: CTT insertion) in U2OS cells by Split-PE2-ΔRH (AAV I+AAV2) and a control (AAV2 only). Split-PE2-ΔRH was delivered via dual-AAV transduction. The AAV expressing the RT and peg/ngRNAs also co-translationally expressed eGFP. One week post-transduction, cells were sorted for top 20-25% GFP MFI and cultured for another 72 h before cell harvest and gDNA extraction (Methods). (n=3; independent replicates).

### DETAILED DESCRIPTION

Prime editing uses CRISPR-guided reverse transcription to enable the programmable introduction of any desired base substitution or small insertion/deletion. Mutations are induced by a PE protein (e.g., PE2) together with a prime editing gRNA (pegRNA) (FIGS. 1A-C). For PE2, the pegRNA directs nSpCas9 activity to create an R-loop with a nicked DNA strand, which anneals to a primer binding sequence (PBS) at the 3′ end of the pegRNA (FIGS. 1A, B). The RT part of the PE protein then reverse transcribes the reverse transcription template (RTT) that is adjacent to the PBS into DNA encoding the desired edit of interest (FIG. 1C). This DNA template then mediates introduction of the edit into the genomic locus by a mechanism that is not yet fully defined. Editing efficiency can be further enhanced with the PE3 system in which an additional secondary nick mediated by a nicking gRNA (ngRNA) is introduced either up- or down-stream of the desired edit site and on the strand opposite the one nicked by the PE protein/pegRNA complex (FIG. 1C)¹. PE3b is a modified version of the PE3 method, in which a nicking guide RNA (ngRNA) is used that binds only the edited DNA sequence.¹ See also³⁰. Recent work has shown that concomitant overexpression of a dominant negative mutant of human MLH1 (termed hMLH1dn), a protein involved in DNA mismatch repair, can further enhance prime editing efficiencies in human cells³⁵. One challenge for use of all prime editing systems is the large size of the required PE2 protein (2117 aa encoded by 6351 bps), a difficulty that is exacerbated if one also needs to encode an additional ngRNA and/or the hMLH1dn protein (753 aa encoded by 2259 bps).

Surprisingly, as shown herein, the RT and nCas9 components of PE proteins functioned efficiently even when separated (FIGS. 1D-G). This has important implications for improving prime editing and better understanding its other potential effects on cells. The present results strongly suggest that with existing intact PE proteins, the RT activity is likely provided by a second PE molecule that is presumably not bound to the target DNA site (i.e., from solution). This in turn implies that the efficiency of prime editing can be further increased by creating different next-generation fusions in which the RT actually does function in cis to the nCas9 (i.e., a configuration in which RT activity is dependent on being tethered to the on-target site, e.g., in the inlaid versions described herein). It also raises the possibility that with existing prime editors, an RT may be able to act from solution on other off-target genomic sites in which a nicked DNA-RNA hybrid might be present, although it is not clear whether such an intermediate actually occurs or would have any biological consequence in human or other cells.

The Split-PEs and reduced size RTs (reduced size relative to MMLV-RT) described herein provide new reagents and architectures that enhance the delivery of prime editing components and accelerate further improvements to the platform. Split-PEs address a limitation imposed by size-constrained AAV vectors—namely that the full-length PE2 protein is currently too large to fit into a single AAV vector. By leveraging the Split-PE architecture, one can encode the nSpCas9 protein in one AAV and the pegRNA/ngRNA and RT in another, thereby creating a configuration in which only cells that are transduced by both vectors will undergo editing without the need for additional components such as split intein sequences used previously with CRISPR nucleases, base editors, and prime editors^{1, 21, 22}. In direct comparisons, the split architecture was more efficient than the previously described split-intein system, most likely because there is no need for the additional step of reconstituting a required protein component in our split configuration. The split-PE system would also be expected to enhance and simplify both RNA and ribonucleoprotein delivery methods due to more efficient expression of shorter-length nCas9 and RT components instead of a full-length fusion of these two components. Finally, the present studies provide proof-of-principle for how the split architecture can facilitate more rapid screening of new prime editor variants with improved properties. Rather than cloning and sequencing a new lengthy fusion for each RT variant and determining where and how to fuse each of these to a nicking Cas9, it is possible to rapidly construct and then screen a large series of different viral, non-viral, and engineered RTs to identify those with desired activities. Similarly, this modularity should also permit the rapid screening of alternative nicking Cas9 or other nickases for prime editing.

### Split Prime Editors

Described herein are compositions and methods for prime editing that make use of CRISPR Cas proteins (preferably nickases, though nucleases can also be used, see Adikusuma et al., Nucleic Acids Res. 2021 Sep. 17; gkab792) and a reverse transcriptase (RT), wherein the nickases and the RT are separate molecular entities, i.e., are not conjugated, fused, or linked together.

The compositions can also include a pegRNA that directs the nickase to a selected genomic target sequence, or nucleic acid comprising a sequence encoding a pegRNA, as well as optionally an ngRNA, or nucleic acid comprising a sequence encoding an ngRNA.

In some embodiments, the compositions comprise nickase and/or RT proteins; alternatively the compositions can comprise nucleic acids encoding the nickase and/or RT. Such nucleic acids can include mRNA or cDNA encoding the proteins, and the nucleic acids can be naked or in an expression vector, e.g., comprising a sequence such as a promoter that drives expression of the protein. The sequence can, for example, be in an expression construct.

In some embodiments, provided herein are prime editors comprising a fusion protein that is cleaved into separate Cas nickase protein and RT protein components following their expression as a single polypeptide (e.g., with the components separated by a protease cleavage site or a 2A self-cleaving peptide sequence).

The fusion proteins can include one or more ‘self-cleaving’ 2A peptides between the coding sequences. 2A peptides are 18-22 amino-acid-long viral peptides that mediate cleavage of polypeptides during translation in eukaryotic cells. 2A peptides include F2A (foot-and-mouth disease virus), E2A (equine rhinitis A virus), P2A (porcine teschovirus-1 2A), and T2A (Thosea asigna virus 2A), and generally comprise the sequence GDVEXNPGP (SEQ ID NO:1) at the C-terminus. See, e.g., Liu et al., Sci Rep. 2017; 7: 2193. The following table provides exemplary 2A sequences.

| | | SEQ ID | |
|---|---|---|---|
| 2A | Coding Sequence | NO: | Source |
| F2A: | GCGCCAGTAAAGCAGACATTAAACTTT | 135 | STEMCCA |
| | GATTTCTGAAACTTGCAGGTGATGTAG | | (PMID: |
| | AGTCAAATCCAGGTCCA | | 20715179 |
| F2A: | GGCAGCGGAAAACAGCTGTTGAATTTTG | 136 | pEB-C5 |
| | ACCTTCTCAAGTTGGCGGGAGACGTGGA | | (PMID: |
| | GTCCAACCCAGGGCCC | | 25772473) |
| P2A: | GCCACTAACTTCTCCCTGTTGAAACAAG | 137 | STEMCCA |
| | CAGGGGATGTCGAAGAGAATCCCGGGCCA | | (PMID: |
| | | | 20715179) |
| E2A: | CAATGTACTAACTACGCTTTGTTGAAAC | 138 | STEMCCA |
| | TCGCTGGCGATGTTGAAAGTAACCCCGG | | (PMID: |
| | TCCT | | 20715179) |
| T2A: | GGGGGGGGGTCCGGAGGAGAGGGCAGAG | 139 | pEB-C5 |
| | GAAGTCTTCTAACATGCGGTGACGTGGA | | (PMID: |
| | GGAGAATCCTGGCCCA | | 25772473) |

Alternatively or in addition, the fusion proteins can include one or more protease-cleavable peptide linkers between the coding sequences. A number of protease-sensitive linkers are known in the art, e.g., comprising furin cleavage sites RX(R/K)R, RKRR (SEQ ID NO:140) or RR, VSQTSKLTRAETVFPDVD (SEQ ID NO:141); EDVVCCSMSY (SEQ ID NO:142); RVLAEA(SEQ ID NO:143); GGGGSSPLGLWAGGGGS (SEQ ID NO:144); TRHRQPRGWEQL (SEQ ID NO:145); MMP 1/9 cleavage sequence PLGLWA (SEQ ID NO:146); TEV Protease sensitive linkers comprising ENLYFQ(G/S) (SEQ ID NO:147); Factor Xa sensitive linkers comprising I(E/D)GR; or LSGRDNH (SEQ ID NO:148) which is cleaved by cancer-associated proteases matriptase, legumain, and uPA. See, e.g., Chen et al., Adv Drug Deliv Rev. 2013 Oct. 15: 65(10): 1357-1369.

### Cas Proteins

The present compositions and methods can use any Cas protein that forms an R loop and nicks on the non-targeted strand. Examples include Cas9 (e.g., SpCas9, SaCas9, and others, e.g., as shown in Table A1). In some embodiments, the Cas protein is Cas12a, Cas12b1, Cas12c, Cas12d, Cas12e, Cas12f, and Cas12j, e.g., as shown in Table A1. The Cas protein is at least 60, 70, 80, 90, 95, 97, 98, or 99% identical to a wild type or variant Cas protein that retains function, i.e., that can bind the target strand, form an R loop, and preferably can induce a nick only on the non-targeted strand, although full nucleases that cut both strands can also be used (see Adikusuma et al., Nucleic Acids Res. 2021 Sep. 17; gkab792).

Although herein we refer to Cas9, in general any Cas9-like nickase could be used (including the related Cpf1/Cas12a enzyme classes), unless specifically indicated.

TABLE A1
List of Exemplary Cas9 or Cas12a Orthologs

| | | | Active |
|---|---|---|---|
| | | Reference/ | sites/catalytic |
| | | Literature | residues (e.g. |
| Orthologue | Accession | (PMID) | RuvC/HNH) |
| S. pyogenes Cas9 | Q99ZW2.1 | WO2014204725, | D10A, E762A, |
| (SpCas9) | | 23907171 & | H840A, D839A, |
| | | 31361218 | N854A, N863A, or |
| | | | D986A |
| S. aureus Cas9 (SaCas9) | J7RUA5.1 | Friedland et al., | D10A and N580 |
| | | Genome Biology | |
| | | 16: 1 (2015) | |
| Streptococcus canis | I7QXF2 | 30397647 | D10, H849 |
| Cas9 (ScCas9) | (Uniprot), | | |
| | WP_003043819 | | |
| | (NCBI) | | |
| S. thermophilus Cas9 | G3ECR1.2 | Gasiunas et al., | D31A and N891A |
| (St1Cas9) | | Proceedings of the | |
| | | National | |
| | | Academy of | |
| | | Sciences, 109: 39 | |
| | | (2012) | |
| S. pasteurianus Cas9 | BAK30384.1 | | D10, H599* |
| (SpaCas9) | | | |
| C. jejuni Cas9 (CjCas9) | Q0P897.1 | Yamada et al., | D8A, H559A |
| | | Molecular Cell, | |
| | | 65: 6 (2017) | |
| F. novicida Cas9 | A0Q5Y3.1 | WO2017/189308, | D11, N995²¹ |
| (FnCas9) | | Zetsche et al., | |
| | | Cell, 163(3): 759- | |
| | | 771 (2015) | |
| P. lavamentivorans | A7HP89.1 | | D8, H601* |
| Cas9 (PlCas9) | | | |
| C. lari Cas9 (ClCas9) | G1UFN3.1 | | D7, H567* |
| Pasteurella multocida | Q9CLT2.1 | | |
| Cas9 | | | |
| F. novicida Cpf1 | A0Q7Q2.1 | WO2017/189308, | D917, E1006, |
| (FnCpf1) | | Zetsche et al., | D1255 |
| | | Cell, 163(3): 759- | |
| | | 771 (2015) | |
| M. bovoculi Cpf1 | WP_052585281.1 | | D986A** |
| (MbCpf1) | | | |
| A. sp. BV3L6 Cpf1 | U2UMQ6.1 | Yamano et al., | D908, 993E, |
| (AsCpf1) | | Cell 165(4): 949- | Q1226, D1263 |
| | | 962 (2016) | |
| L. bacterium N2006 | A0A182DWE3.1 | Tang et al., Nature | D832A |
| (LbCpf1) | | Plants, 3(7): 17103 | |
| | | (2017) | |
| Streptococcus macacae | G5JVJ9 (Uniprot) | 32424114 | D10, H842 |
| Cas9 (SmacCas9) | WP_003079701 | | |
| | (NCBI) | | |
| Streptococcus mutans | Q8DTE3 | 32150575, | D10, H840 |
| (SmutCas9) | (Uniprot); | 32424114 | |
| | BAQ19582 | | |
| | WP_024784288 | | |
| | (both NCBI) | | |
| Streptococcus | G3ECR1 | 31900288 | D31, H868 |
| thermophilus (St1Cas9) | (Uniprot); | | |
| Streptococcus | Q03LF7 | 31900288 | D9, H599 |
| thermophilus (strain | (Uniprot); | | |
| ATCC BAA-491/LMD- | WP_014621379 | | |
| 9) Cas9-1 | (NCBI) | | |
| Streptococcus sanguinis | F3UXG6 | | D13, H896 |
| SK49 Cas9 | (Uniprot) | | |
| Streptococcus sanguinis | E8KPA4 | | H642 (HNH) |
| VMC66 Cas9 | (Uniprot) | | |
| Streptococcus sanguinis | F0I6Z8 (Uniprot) | | D10, H842 |
| SK115 Cas9 | | | |
| Streptococcus sanguinis | F0FD37 | | D10, H842 |
| SK353 Cas9 | (Uniprot) | | |
| Streptococcus sanguinis | F2C4I5 (Uniprot) | | D11, H843 |
| SK330 Cas9 | | | |
| Streptococcus sanguinis | A0A7H8V0N3 | | D11, H851 |
| Cas9 | (Uniprot) | | |
| Streptococcus equinis | | | |
| Cas9 | | | |
| Streptococcus oralis | A0A1X1HQZ5 | | D11, H843 |
| subsp. oralis Cas9 | | | |
| Streptococcus | WP_049510439, | 32424114 | |
| pseudopneumoniae | WP_049538452 | | |
| Cas9 (SudoCas9) | (both NCBI) | | |
| Staphylococcus aureus | J7RUA5 | 25830891 | D10, H557 |
| Cas9 (SaCas9) | (Uniprot) | | (HNH), N580 |
| | | | (HNH) |
| Campylobacter jejuni | Q0P897 (Uniprot) | 28220790 | D8, H559 |
| Cas9 (CjCas9) | | | |
| Neisseria meningitidis 1 | A1IQ68 (Uniprot) | 24076762 | D16, H588 |
| Cas9 (Nme1Cas9) | 6JDQ (PDB) | | |
| Neisseria meningitidis 2 | 6JFU (PDB) | 30581144 | D16, H588 |
| Cas9 (Nme2Cas9) | WP_002230835.1 | | |
| | (NCBI) | | |

These orthologs, and mutants and variants thereof as known in the art, can be used in any of the fusion proteins, systems, compositions, or methods described herein. See, e.g., WO 2017/040348 (which describes variants of SaCas9 and SpCas 9 with increased specificity) and WO 2016/141224 (which describes variants of SaCas9 and SpCas 9 with altered PAM specificity).

The Cas9 nuclease from S. pyrogenes (hereafter simply Cas9) can be guided via simple base pair complementarity between 17-20 nucleotides of an engineered guide RNA (gRNA). e.g., a single guide RNA or crRNA/tracrRNA pair, and the complementary strand of a target genomic DNA sequence of interest that lies next to a protospacer adjacent motif (PAM), e.g., a PAM matching the sequence NGG or NAG (Shen et al., Cell Res (2013); Dicarlo et al., Nucleic Acids Res (2013); Jiang et al., Nat Biotechnol 31, 233-239 (2013); Jinek et al., Elife 2, e00471 (2013); Hwang et al., Nat Biotechnol 31, 227-229 (2013); Cong et al., Science 339, 819-823 (2013); Mali et al., Science 339, 823-826 (2013c); Cho et al., Nat Biotechnol 31, 230-232 (2013); Jinek et al., Science 337, 816-821 (2012)). The engineered CRISPR from Prevotella and Francisella 1 (Cpf1, also known as Cas12a) nuclease can also be used, e.g., as described in Zetsche et al., Cell 163, 759-771 (2015); Schunder et al., Int J Med Microbiol 303, 51-60 (2013); Makarova et al., Nat Rev Microbiol 13, 722-736 (2015); Fagerlund et al., Genome Biol 16, 251 (2015). Unlike SpCas9, Cpf1/Cas12a requires only a single 42-nt crRNA, which has 23 nt at its 3′ end that are complementary to the protospacer of the target DNA sequence (Zetsche et al., 2015). Furthermore, whereas SpCas9 recognizes an NGG PAM sequence that is 3′ of the protospacer, AsCpf1 and LbCp1 recognize TITN PAMs that are found 5′ of the protospacer (Id.).

In some embodiments, the present system utilizes a wild type or variant Cas9 protein, e.g., as noted above, optionally from S. pyogenes or Staphylococcus aureus, or a wild type or variant Cpf1 protein from Acidaminococcus sp. BV3L6 or Lachnospiraceae bacterium ND2006, either as encoded in bacteria (i.e., wild type) or codon-optimized for expression in mammalian cells and/or modified in its PAM recognition specificity and/or its genome-wide specificity. A number of variants of Cas9 have been described; see, e.g., WO 2016/141224, PCT/US2016/049147, Kleinstiver et al., Nat Biotechnol. 2016 August; 34(8); 869-74; Tsai and Joung, Nat Rev Genet. 2016 May; 17(5): 300-12; Kleinstiver et al., Nature. 2016 Jan. 28; 529(7587): 490-5; Shmakov et al., Mol Cell. 2015 Nov. 5:60(3): 385-97: Kleinstiver et al., Nat Biotechnol. 2015 December; 33(12): 1293-1298; Dahlman et al., Nat Biotechnol. 2015 November; 33(11): 1159-61; Kleinstiver et al., Nature. 2015 Jul. 23; 523(7561): 481-5; Wyvekens et al., Hum Gene Ther. 2015 July; 26(7): 425-31; Hwang et al., Methods Mol Biol. 2015; 1311:317-34; Osborn et al., Hum Gene Ther. 2015 February:26(2): 114-26; Konermann et al., Nature. 2015 Jan. 29; 517(7536): 583-8; Fu et al., Methods Enzymol. 2014; 546:21-45; and Tsai et al., Nat Biotechnol. 2014 June; 32(6): 569-76, inter alia. Some of the above, and additional variants, are listed in Table A2. The guide RNA is expressed or present in the cell together with the Cas9 or Cpf1. Either the guide RNA or the nuclease, or both, can be expressed transiently or stably in the cell or introduced as a purified protein or nucleic acid.

In some embodiments, the Cas9 also includes one of the following mutations, which reduce nuclease activity of the Cas9; e.g., for SpCas9, mutations at D10A or H840A (which creates a single-strand nickase).

In some embodiments, the SpCas9 variants also include mutations at one of the following amino acid positions, to reduce the nuclease activity of the Cas9 to create a nickase: D10, E762, D839, H983, or D986 and H840 or N863, preferably H840A. D839A, or N863A, to render the nuclease portion of the protein catalytically inactive; substitutions at these positions could be alanine (as they are in Nishimasu al., Cell 156, 935-949 (2014)), or other residues, e.g., glutamine, asparagine, tyrosine, serine, or aspartate, e.g., E762Q, H983N, H983Y, D986N, N863D, N863S, or N863H (see WO 2014/152432).

In some embodiments, the Cas9 is fused to one or more SV40 or bipartite (bp) nuclear localization sequences (NLSs) protein sequences; an exemplary (bp)NLS sequence is as follows: (KRTADGSEFES)PKKKRKV (SEQ ID NO: 149). Typically, the NLSs are at the N- and C-termini of an ABEmax fusion protein, but can also be positioned at the N- or C-terminus in other ABEs, or between the DNA binding domain and the deaminase domain. Linkers as known in the art can be used to separate domains.

TABLE A2
List of Exemplary High Fidelity and/or PAM-relaxed RGN Orthologs

| Published | | |
|---|---|---|
| HF/PAM-RGN | | |
| variants | PMID/Reference | Mutations* |
| S. pyogenes Cas9 | 26628643 | K810A/K1003A/R1060A (1.0); |
| (SpCas9) | | K848A/K1003A/R1060A(1.1) |
| eSpCas9 | | |
| S. pyogenes Cas9 | 29431739 | M495V/Y515N/K526E/R661Q; |
| (SpCas9) | | (M495V/Y515N/K526E/R661S; |
| evoCas9 | | M495V/Y515N/K526E/R661L) |
| S. pyogenes Cas9 | 26735016 | N497A/R661A/Q695A/Q926A |
| (SpCas9) HF1 | | |
| S. pyogenes Cas9 | 30082871 | R691A |
| (SpCas9) HiFi | | |
| Cas9 | | |
| S. pyogenes Cas9 | 28931002 | N692A, M694A, Q695A, H698A |
| (SpCas9) | | |
| HypaCas9 | | |
| S. pyogenes Cas9 | 30082838 | F539S, M763I, K890N |
| (SpCas9) | | |
| Sniper-Cas9 | | |
| S. pyogenes Cas9 | 29512652 | A262T, R324L, S409I, E480K, E543D, M694I, |
| (SpCas9) xCas9 | | E1219V |
| S. pyogenes Cas9 | 30166441 | R1335V, L1111R, D1135V, G1218R, |
| (SpCas9) | | E1219F, A1322R, T1337R |
| SpCas9-NG | | |
| S. pyogenes Cas9 | 26098369 | D1135V, R1335Q, T1337R; |
| (SpCas9) | | D1135V/G1218R/R1335E/T1337R |
| VQR/VRER | | |
| S. aureus Cas9 | 26524662 | E782K/N968K/R1015H |
| (SaCas9)-KKH | | |
| enAsCas12a | USSN 15/960,271 | One or more of: E174R, S170R, S542R, K548R, |
| | | K548V, N551R, N552R, K607R, K607H, e.g., |
| | | E174R/S542R/K548R, E174R/S542R/K607R, |
| | | E174R/S542R/K548V/N552R, |
| | | S170R/S542R/K548R, S170R/E174R, |
| | | E174R/S542R, S170R/S542R, |
| | | E174R/S542R/K548R/N551R, |
| | | E174R/S542R/K607H, S170R/S542R/K607R, or |
| | | S170R/S542R/K548V/N552R |
| enAsCas12a-HF | USSN 15/960,271 | One or more of: B174R, S542R, K548R, e.g., |
| | | E174R/S542R/K548R, E174R/S542R/K607R, |
| | | E174R/S542R/K548V/N552R, |
| | | S170R/S542R/K548R, S170R/E174R, |
| | | E174R/S542R, S170R/S542R, |
| | | E174R/S542R/K548R/N551R, |
| | | E174R/SS42R/K607H, S170R/S542R/K607R, or |
| | | S170R/S542R/K548V/N552R, with the addition of |
| | | one or more of: N282A, T315A, N515A and K949A |
| enLbCas12a(HF) | USSN 15/960,271 | One or more of T152R, T152K, D156R, D156K, |
| | | Q529K, G532R, G532K, G532Q, K538R, K538V, |
| | | DS41R, Y542R, M592A, K595R, K595H, K595S or |
| | | K595Q, e.g., D156R/G532R/K538R, |
| | | D156R/G532R/K595R, |
| | | D156R/G532R/K538V/Y542R, |
| | | T152R/G532R/K538R, T152R/D156R, |
| | | D156R/G532R, T152R/G532R, |
| | | D156R/G532R/K538R/D541R, |
| | | D156R/G532R/K59SH, T152R/G532R/K595R, |
| | | T152R/G532R/K538V/Y542R, optionally with the |
| | | addition of one or more of: N260A, N256A, K514A, |
| | | D505A, K881A, S286A, K272A, K897A |
| enFnCas12a(HF) | USSN 15/960,271 | One or more of T177A, K180R, K180K, E184R, |
| | | E184K, T604K, N607R, N607K, N607Q, K613R, |
| | | K613V, D616R, N617R, M668A, K671R, K671H, |
| | | K671S, or K671Q, e.g., E184R/N607R/K613R, |
| | | E184R/N607R/K671R, |
| | | E184R/N607R/K613V/N617R, |
| | | K180R/N607R/K613R, K180R/E184R, |
| | | E184R/N607R, K180R/N607R, |
| | | E184R/N607R/K613R/D616R, |
| | | E184R/N607R/K671H, K180R/N607R/K671R, |
| | | K180R/N607R/K613V/N617R, optionally with the |
| | | addition of one or more of: N305A, N301A, K589A, |
| | | N580A, K962A, S334A, K320A, K978A |
| chimeric Cas9 | 30718489 | S. aureus Cas9 with PAM interaction domain from |
| cCas9 | | SaCas9 orthologues, expands recognition and |
| | | targetability of NNVRRN, NNVACT, NNVATG, |
| | | NNVATT, NNVGCT, NNVGTG, and NNVGTT |
| | | PAM sequences |
| Streptococcus | doi: https://doi.org/ | Recognizes 5′-NAA-3′ PAM |
| macacae (Smac) | 10.1101/429654 | |
| Cas9 NCTC | | |
| 11558 | | |
| Spy-mac Cas9, | doi: https://doi.org/ | Recognizes 5′-NAA-3′ PAM |
| Smac-py Cas9 | 10.1101/429654 | |
| N. meningitidis | 30581144 | Recognizes N₄CC PAM |
| Nme2Cas9 | | |
| S. pyogenes Cas9 | 32217751 | D1135L/S1136W/G1218K/E1219Q/R1335Q/T1337R |
| (SpCas9) | | |
| SpCas9-SpG | | |
| S. pyogenes Cas9 | 32217751 | A61R/L1111R/D1135L/S1136W/G1218K/E1219Q/ |
| (SpCas9) | | N1317R/A1322R/R1333P/R1335Q/T1337R |
| SpCas9-SpRY | | |
| Engineered | 36076084 | P6S, E33G, K104T, D152A, F260L, A263T, A303S, |
| N. meningitidis | | D451V, E520A, R646S, F696V, G711R, I758V, |
| Nme2Cas9 | | H767Y, E932K, N1031S, R1033G, K1044R, |
| eNme2-C | | Q1047R, V1056A |
| (N₄CN PAM) | | |
| Engineered | 36076084 | S6P, G33E, A520E, S646R, V696F, R711G, V758I, |
| N. meningitidis | | Y767H |
| Nme2Cas9 | | |
| eNme2-C.NR | | |
| (N₄CN PAM) | | |
| Engineered | 36076084 | E47K, V68M, T123A, D152G, E154K, T396A, |
| N. meningitidis | | H413N, A427S, H452R, E460A, A484T, S629P, |
| Nme2Cas9 | | N674S, D720A, V765A, H767Y, H771R, V821A, |
| eNme2-T1 | | D844A, I859V, W865L, M951R, K1005R, D1028N, |
| (N₄TN PAM) | | S1029A, R1033Y, R1049S, N1064S |
| Engineered | 36076084 | E47K, R63K, V68M, A116T, T123A, D152N, |
| N. meningitidis | | E154K, E221D, T396A, H452R, E460K, N674S, |
| Nme2Cas9 | | D720A, A724S, K769R, S816I, D844A, E932K, |
| eNme2-T2 | | K940R, M951R, K1005R, D1028N, S1029A, |
| (N₄TN PAM) | | R1033N, R1049C, L1075M |

*predicted based on UniRule annotation on the UniProt database.

### Reverse Transcriptases (RTs), Reduced Size RTs, and Variant RTs

The present compositions and methods can use any RT, including Group II introns. Group II introns are retroelements that consist of a self-splicing ribozyme and an intron encoded protein (IEP) which functions as a reverse transcriptase (RT). DNA endonuclease, and RNA maturase. Exemplary alternative RTs include those listed in Table B.

As noted above, PE2 includes a pentamutant Moloney Murine Leukemia Virus reverse transcriptase (MMLV-RT) fused at its C-terminus. The group II intron RT (commercially available as “MarathonRT”) from Eubacterium rectale (E.r.) has been shown to display superior intrinsic RT processivity compared to Superscript IV. As shown herein, substitution of the M-MLV RT in a PE with MarathonRT or other RTs resulted in efficient prime editing in the HEK293T cell line. Thus, provided herein are prime editors, both split, fusion, and inlaid, that include RTs other than MMLV-RT, e.g., as shown herein, e.g., in Table B, FIG. 7, or FIG. 12, or variants thereof.

TABLE B
Alternative reverse transcriptases

| | NCBI or Uniprot | Reverse |
|---|---|---|
| Organism | Acc. No. or Source | Transcriptase Type |
| Geobacillus | E2GM63 (uniport) | Group II Intron |
| stearothermophilus* | | |
| Lactococcus lactis | AAB06503.1 | Group II Intron |
| subsp. lactis | | |
| Thermosynechococcus | BAC08171.1 | Group II Intron |
| elongatus BP-1 | | |
| Sinorhizobium meliloti | WP_010967953.1 | Group II Intron |
| Methanosarcina | AAM07961.1 | Group II Intron |
| acetivorans C2A | | |
| Enterobacter cloacae | AEC33268.1 | Group II Intron |
| Clostridium | NP_350100.1 | Group II Intron |
| acetobutylicum ATCC | | |
| 824 | | |
| Bacillus halodurans | BAA90841.1 | Group II Intron |
| Pseudomonas | AAB68949.1 | Group II Intron |
| alcaligenes | | |
| Pseudomonas putida | CAB81565.1 | Group II Intron |
| Streptococcus | CAC35989.1 | Group II Intron |
| agalactiae | | |
| Roseburia intestinalis | D4L313 (uniprot) | Group II Intron |
| Eubacterium rectale | CBK92290.1 | Group II Intron |
| (marathonRT) | | |
| Streptococcus | WP_013851921.1 | Group II Intron |
| pasteurianus | | |
| Shigella sonnei | WP_077124660.1 | Group II Intron |
| Saccharomyces | NP_009310.1 | Group II Intron |
| cerevisiae S288C | | (yeast) |
| (yeast) | | |
| Saccharomyces | NP_009309.1 | Group II Intron |
| cerevisiae S288C | | (yeast) |
| (yeast) | | |
| Bordetella virus BPP1 | AAR97672.1 | Diversity Generating |
| | | Retroelement |
| ANMV-1 virus | AJP62064.1 | Diversity Generating |
| | | Retroelement |
| Bacteroides phage p00 | DAC76693.1 | Diversity Generating |
| | | Retroelement |
| Treponema denticola | AAS12785.1 | Diversity Generating |
| ATCC 35405 | | Retroelement |
| archacon | AJF63168.1 | Diversity Generating |
| GW2011_AR20 | | Retroelement |
| Baboon endogenous | YP_009109694.1 | Retrovirus |
| virus strain M7 | | |
| Feline leukemia virus | NP_047255.1 | Retrovirus |
| Human foamy virus | CAA68999.1 | Retrovirus |
| Feline | AAB59937.1 | Retrovirus |
| immunodeficiency | | |
| virus | | |
| Human Endogenous | Nam Lee, et. al (2007) | Retrovirus |
| Retrovirus K | | |
| (reconstituted) | | |
| Necator americanus | XP_013295720.1 | Group II intron |
| | | (eukaryotic) |
| Axinella verrucosa | CRX66588.1 | Group II intron |
| | | (eukaryotic) |
| Axinella verrucosa | CRX66589.1 | Group II intron |
| | | (eukaryotic) |
| Xenopolymerase RTX | Jared W. Ellefson, et. | Thermococcus |
| | al (2016) | kodakarensis |
| | | (engineered) |

*Geobacillus stearothermophilus GsI-IIC intron RT (denoted GsI-IIC RT; sold commercially as TGIRT-III; InGex); see Stamos et al., Mol Cell. 2017 Dec. 7; 68(5): 926-939.e4.

Exemplary RT sequences include:

Eubacterium rectale RT (aka Marathon-RT; WT)
SEQ ID NO: 35
MDTSNLMEQILSSDNLNRAYLQVVRNKGAEGVDGMKYTELKEHLAK
NGETIKGQLRTRKYKPQPARRVEIPKPDGGVRNLGVPTVTDRFIQQAI
AQVLTPIYEEQFHDHSYGFRPNRCAQQAILTALNIMNDGNDWIVDIDL
EKFFDTVNHDKLMTLIGRTIKDGDVISIVRKYLVSGIMIDDEYEDSIVG
TPQGGNLSPLLANIMLNELDKEMEKRGLNFVRYADDCIIMVGSEMSA
NRVMRNISRFIEEKLGLKVNMTKSKVDRPSGLKYLGFGFYFDPRAHQF
KAKPHAKSVAKFKKRMKELTCRSWGVSNSYKVEKLNQLIRGWINYF
KIGSMKTLCKELDSRIRYRLRMCIWKQWKTPQNQEKNLVKLGIDRNT
ARRVAYTGKRIAYVCNKGAVNVAISNKRLASFGLISMLDYYIEKCVTC
Human endogenous retrovirus K consensus (HERV-
Kcon) RT
SEQ ID NO: 36
MKSRKRRNRVSFLGAATVEPPKPIPLTWKTEKPVWVNQWPLPKQKLE
ALHLLANEQLEKGHIEPSFSPWNSPVFVIQKKSGKWRMLTDLRAVNA
VIQPMGPLQPGLPSPAMIPKDWPLIIIDLKDCFFTIPLAEQDCEKFAFT
IPAINNKEPATRFQWKVLPQGMLNSPTICQTFVGRALQPVREKFSDCYI
IHYIDDILCAAETKDKLIDCYTFLQAEVANAGLAIASDKIQTSTPFHYL
GMQIENRKIKPQKIEIRKDTLKTLNDFQKLLGDINWIRPTLGIPTYAMS
NLFSILRGDSDLNSKRMLTPEATKEIKLVEEKIQSAQINRIDPLAPLQL
LIFATAHSPTGIIIQNTDLVEWSFLPHSTVKTFTLYLDQIATLIGQTRL
RIIKLCGNDPDKIVVPLTKEQVRQAFINSGAWQIGLANFVGIIDNHYPK
TKIFQFLKLTTWILPKITRREPLENALTVFTDGSSNGKAAYTGPKERVI
KTPYQSAQRAELVAVITVLQDFDQPINIISDSAYVVQATRDVETALIKY
SMDDQLNQLFNLLQQTVRKRNFPFYITHIRAHTNLPGPLTKANEQADLL
VSSALIKAQELHA
Geobacillus stearothermophilus GsI-IIC RT (WT)
SEQ ID NO: 37
MALLERILARDNLITALKRVEANQGAPGIDGVSTDQLRDYIRAHWSTI
HAQLLAGTYRPAPVRRVEIPKPGGGTRQLGIPTVVDRLIQQAILQELTP
IFDPDFSSSSFGFRPGRNAHDAVRQAQGYIQEGYRYVVDMDLEKFFDR
VNHDILMSRVARKVKDKRVLKLIRAYLQAGVMIEGVKVQTEEGTPQG
GPLSPLLANILLDDLDKELEKRGLKFCRYADDCNIYVKSLRAGQRVKQ
SIQRFLEKTLKLKVNEEKSAVDRPWKRAFLGFSFTPERKARIRLAPRSI
QRLKQRIRQLTNPNWSISMPERIHRVNQYVMGWIGYFRLVETPSVLQT
IEGWIRRRLRLCQWLQWKRVRTRIRELRALGLKETAVMEIANTRKGA
WRTTKTPQLHQALGKTYWTAQGLKSLTQRYFELRQG

Geobacillus stearothermophilus GisI-IIC intron RT (GisI-IIC RT) pentamutants can also be used, e.g., comprising mutations D11IR/N23R/G71R/G113K/P194R (positions bolded in SEQ ID NO:37, above.

Exemplary MMLV RT sequences include the following:

MMLV-RT pentamutant (used in classic PE2),
without NLS, starts with T (not M)
SEQ ID NO: 38
TLNIEDEYRLHETSKEPDVSLGSTWLSDFPQAWAETGGMGLAVRQAP
LIIPLKATSTPVSIKQYPMSQEARLGIKPHIQRLLDQGILVPCQSPWNT
PLLPVKKPGTNDYRPVQDLREVNKRVEDIHPTVPNPYNLLSGLPPSHQW
YTVLDLKDAFFCLRLHPTSQPLFAFEWRDPEMGISGQLTWTRLPQGFK
NSPTLFNEALHRDLADFRIQHPDLILLQYVDDLLLAATSELDCQQGTR
ALLQTLGNLGYRASAKKAQICQKQVKYLGYLLKEGQRWLTEARKET
VMGQPTPKTPRQLREFLGKAGFCRLFIPGFAEMAAPLYPLTKPGTLFN
WGPDQQKAYQEIKQALLTAPALGLPDLTKPFELFVDEKQGYAKGVLT
QKLGPWRRPVAYLSKKLDPVAAGWPPCLRMVAAIAVLTKDAGKLTM
GQPLVILAPHAVEALVKQPPDRWLSNARMTHYQALLLDTDRVQFGPV
VALNPATLLPLPEEGLQHNCLDILAEAHGTRPDLTDQPLPDADHTWYT
DGSSLLQEGQRKAGAAVTTETEVIWAKALPAGTSAQRAELIALTQAL
KMAEGKKLNVYTDSRYAFATAHIHGEIYRRRGWLTSEGKEIKNKDEIL
ALLKALFLPKRLSIIHCPGHQKGHSAEARGNRMADQAARKAAITETPD
TSTLLIENSSP

The present compositions and methods can make use of variants as known in the art and as provided herein. e.g., MarathonRT, GsI-IIC RT, and MMLV-RT variants.

Table C provides a list of Marathon variants with altered prime editing efficiencies at three endogenous target sites:

TABLE C
Marathon Variants

| | Lower/higher prime editing efficiency |
|---|---|
| Variant | compared to WT Marathon-RT |
| D14K | Same |
| D14R | Same or + |
| Q22K | ++ |
| Q22R | ++ |
| N26K | ++ |
| N26R | ++ |
| E30K | + |
| E30R | + |
| D74K | ++ |
| D74R | ++ |
| Q91K | Same |
| Q91R | Same to slightly lower (+/−) |
| Q92K | ++ |
| Q92R | + |
| Q96K | ++ |
| Q96R | ++ |
| N116K | ++ |
| N116R | ++ |
| N197K | ++ |
| N197R | ++ |
| E304K | ++ |
| E304R | ++ |
| E319K | Much lower (− − −) |
| E319R | Much lower (− − −) |
| N322K | Much lower (− − −) |
| N322R | Much lower (− − −) |
| N330K | Much lower (− − −) |
| N330R | Much lower (− − −) |
| E422K | + |
| E422R | Same |
| Q91K-Q92K | Same |
| Q91R-Q92R | Same |
| D14R-D74R | ++ |
| D74R-E422K | ++ |
| D14R-D74R-E422K | ++ |
| D14R-N26R-D74R | +++ |
| D14R-D74R-N116K | +++ |
| D14R-D74R-N197R | +++ |
| D14R-N26R-D74R-N116K | ++++ |
| D14R-D74R-N116K-N197R | ++++ |
| D14R-N26R-D74R-E422K | ++ |
| D14R-D74R-Q96R-E422K | ++ |
| D14R-D74R-N116K-E422K | ++ |
| D14R-D74R-N197K-E422K | ++ |
| D14R-N26R-D74R-N197R | ++++ |
| D14R-N26R-D74R-N116K-N197R | +++++ |
| D14R-N26R-D74R-Q96R-N116K-N197R | ++ |
| D14R-N26R-D74R-Q96R-N116K-N197R-E422K | ++ |

Also described herein are reduced size RTs, also referred to as truncation variants. For example, provided are MMLV-RT pentamutant truncation variants comprising one of the following sequences, or a variant thereof, with up to 1, 2, 3, 4, 5, 6, 7, 8, 9, 10, 11, 12, 13, 14, 15, 16, 17, 18, 19, or 20 additional amino acids on the N terminus from the original MMLV-RT, and/or up to 1, 2, 3, 4, 5, 6, 7, 8, 9, 10, 11, 12, 13, 14, 15, 16, 17, 18, 19, 20, 25, 50, 100, 150, or 175 aa on the C terminus from the original MMLV-RT (i.e., reducing the size of the truncation on either end); and/or additional amino acids truncated from either end, e.g., up to 1, 2, 3, 4, 5, 6, 7, 8, 9, or 10 additional amino acids (i.e., for a total of 24, 25, 26, 27, 28, 29, 30, 31, 32, 33, or 34 amino acids) removed from the N terminus and/or up to 1, 2, 3, 4, 5, 6, 7, 8, 9, 10, 11, 12, 13, 14, 15, 16, 17, 18, 19, 20, 25, or 26 aa removed from the C terminus (i.e., for a total of 182, 183, 184, 185, 186, 187, 188, 189, 190, 191, 192, 193, 194, 195, 196, 197, 198, 199, 200, 201, 202, 203, 204, 205, 206, or 207 amino acids removed from the C terminus). Fusions with sequences from other, non-MMLV-RT proteins on the N or C terminus can also be used.

N-terminal truncation (truncation 2 in screen)
(del 23 aa)
SEQ ID NO: 39
TWLSDFPQAWAETGGMGLAVRQAPLIIPLKATSTPVSIKQYPMSQEAR
LGIKPHIQRLLDQGILVPCQSPWNTPLLPVKKPGTNDYRPVQDLREVN
KRVEDIHPTVPNPYNLLSGLPPSHQWYTVLDLKDAFFCLRLHPTSQPLF
AFEWRDPEMGISGQLTWTRLPQGFKNSPTLFNEALHRDLADFRIQHPD
LILLQYVDDLLLAATSELDCQQGTRALLQTLGNLGYRASAKKAQICQ
KQVKYLGYLLKEGQRWLTEARKETVMGQPTPKTPRQLREFLGKAGF
CRLFIPGFAEMAAPLYPLTKPGTLFNWGPDQQKAYQEIKQALLTAPAL
GLPDLTKPFELFVDEKQGYAKGVLTQKLGPWRRPVAYLSKKLDPVAA
GWPPCLRMVAAIAVLTKDAGKLTMGQPLVILAPHAVEALVKQPPDR
WLSNARMTHYQALLLDTDRVQFGPVVALNPATLLPLPEEGLQHNCLD
ILAEAHGTRPDLTDQPLPDADHTWYTDGSSLLQEGQRKAGAAVTTET
EVIWAKALPAGTSAQRAELIALTQALKMAEGKKLNVYTDSRYAFATA
HIHGEIYRRRGWLTSEGKEIKNKDEILALLKALFLPKRLSIIHCPGHQK
GHSAEARGNRMADQAARKAAITETPDTSTLLIENSSP
C-terminal truncation (truncation 5 in screen)
(del 181 aa)
SEQ ID NO: 40
TLNIEDEYRLHETSKEPDVSLGSTWLSDFPQAWAETGGMGLAVRQAP
LIIPLKATSTPVSIKQYPMSQEARLGIKPHIQRLLDQGILVPCQSPWNT
PLLPVKKPGTNDYRPVQDLREVNKRVEDIHPTVPNPYNLLSGLPPSHQW
YTVLDLKDAFFCLRLHPTSQPLFAFEWRDPEMGISGQLTWTRLPQGFK
NSPTLFNEALHRDLADFRIQHPDLILLQYVDDLLLAATSELDCQQGTR
ALLQTLGNLGYRASAKKAQICQKQVKYLGYLLKEGQRWLTEARKET
VMGQPTPKTPRQLREFLGKAGFCRLFIPGFAEMAAPLYPLTKPGTLFN
WGPDQQKAYQEIKQALLTAPALGLPDLTKPFELFVDEKQGYAKGVLT
QKLGPWRRPVAYLSKKLDPVAAGWPPCLRMVAAIAVLTKDAGKLTM
GQPLVILAPHAVEALVKQPPDRWLSNARMTHYQALLLDTDRVQFGPV
VALNPATLLPLPEEGLQHNCL
N- and C-terminal truncation (truncation 6 in
screen) (del 23 AA on N and 181 aa on C)
SEQ ID NO: 41
TWLSDFPQAWAETGGMGLAVRQAPLIIPLKATSTPVSIKQYPMSQEAR
LGIKPHIQRLLDQGILVPCQSPWNTPLLPVKKPGTNDYRPVQDLREVN
KRVEDIHPTVPNPYNLLSGLPPSHQWYTVLDLKDAFFCLRLHPTSQPLF
AFEWRDPEMGISGQLTWTRLPQGFKNSPTLFNEALHRDLADFRIQHPD
LILLQYVDDLLLAATSELDCQQGTRALLQTLGNLGYRASAKKAQICQ
KQVKYLGYLLKEGQRWLTEARKETVMGQPTPKTPRQLREFLGKAGF
CRLFIPGFAEMAAPLYPLTKPGTLFNWGPDQQKAYQEIKQALLTAPAL
GLPDLTKPFELFVDEKQGYAKGVLTQKLGPWRRPVAYLSKKLDPVAA
GWPPCLRMVAAIAVLTKDAGKLTMGQPLVILAPHAVEALVKQPPDR
WLSNARMTHYQALLLDTDRVQFGPVVALNPATLLPLPEEGLQHNCL

In embodiments where a variant or reduced size RT is used, the RT can be separate as described above, or can be tethered to the N terminus or the C terminus of the Cas (e.g., via a linker, e.g., a 32AA or 33AA linker from BE4, ABE, and PE comprising a modified XTEN sequence at the core with flanking GSSG linkers on the side, e.g., as described in Gaudelli et al., Nature 551:464-471 (2017); Komor et al., Science Advances 3(8):eaao4774 (2017); Scholefield et al., Gene Therapy 28:396-401 (2021); Anzalone et al., Nature 576:149-157 (2019); Hsu et al., Nature Communications 12:1034 (2021); WO/2020/191246; WO/2020/191249; WO/2020/191243; WO/2020/191241; WO/2020/191248; WO/2020/191245; WO/2020/191239; WO/2020/191171; WO/2020/191153; WO/2020/191234; WO/2020/191233; and WO/2020/191242), or can be inserted internally, e.g., as described for inlaid BEs: Chu et al., CRISPR J. 2021 April; 4(2): 169-177; Liu et al., Nature Communications 11:6073 (2020); Nguyen Tran et al., Nature Communications 11: 4871 (2020); Li et al., Nature Communications 11:5827 (2020); Wang et al., Signal Transduct. Target. Ther. 4:36 (2019) (site 1055 (between G1055 and E1056) and 2) site 1247 (between G1247 and S1248) of SpCas9) as shown in FIG. 13, or between 535-536; 770-771; 793-794; 801-802; 905-906; 919-920; 1029-1030; or by replacing residues 1048-1063 with the RT domain. Preferably, the inlaid RT domains are flanked with linkers (e.g., 20-50 amino acids, e.g., 30-35 amino acids, e.g., 32-33 amino acids, e.g., 32 amino acid modified XTEN with flanking GlySer linkers). In some embodiments, the RT is inlaid into the PAM interacting domain (PID) or RuvC domain.

Exemplary inlaid prime editors include the following:

Inlaid MMLV-RT in SpCas9 variant 1 (G1055/E1056; no NLS; RT with
flanking 32 AA linkers)
SEQ ID NO: 42
MDKKYSIGLDIGTNSVGWAVITDEYKVPSKKFKVLGNTDRHSI
KKNLIGALLFDSGETAEATRLKRTARRRYTRRKNRICYLQEIFSNEMA
KVDDSFFHRLEESFLVEEDKKHERHPIFGNIVDEVAYHEKYPTIYHLRK
KLVDSTDKADLRLIYLALAHMIKFRGHFLIEGDLNPDNSDVDKLFIQL
VQTYNQLFEENPINASGVDAKAILSARLSKSRRLENLIAQLPGEKKNGL
FGNLIALSLGLTPNFKSNFDLAEDAKLQLSKDTYDDDLDNLLAQIGDQ
YADLFLAAKNLSDAILLSDILRVNTEITKAPLSASMIKRYDEHHQDLTL
LKALVRQQLPEKYKEIFFDQSKNGYAGYIDGGASQEEFYKFIKPILEK
MDGTEELLVKLNREDLLRKQRTFDNGSIPHQIHLGELHAILRRQEDFY
PFLKDNREKIEKILTFRIPYYVGPLARGNSRFAWMTRKSEETITPWNFE
EVVDKGASAQSFIERMTNFDKNLPNEKVLPKHSLLYEYFTVYNELTK
VKYVTEGMRKPAFLSGEQKKAIVDLLFKTNRKVTVKQLKEDYFKKIE
CFDSVEISGVEDRFNASLGTYHDLLKIIKDKDFLDNEENEDILEDIVLTL
TLFEDREMIEERLKTYAHLFDDKVMKQLKRRRYTGWGRLSRKLINGI
RDKQSGKTILDFLKSDGFANRNFMQLIHDDSLTFKEDIQKAQVSGQGD
SLHEHIANLAGSPAIKKGILQTVKVVDELVKVMGRHKPENIVIEMARE
NQTTQKGQKNSRERMKRIEEGIKELGSQILKEHPVENTQLQNEKLYLY
YLQNGRDMYVDQELDINRLSDYDVDAIVPQSFLKDDSIDNKVLTRSD
KNRGKSDNVPSEEVVKKMKNYWRQLLNAKLITQRKFDNLTKAERGG
LSELDKAGFIKRQLVETRQITKHVAQILDSRMNTKYDENDKLIREVKVI
TLKSKLVSDFRKDFQFYKVREINNYHHAHDAYLNAVVGTALIKKYPK
LESEFVYGDYKVYDVRKMIAKSEQEIGKATAKYFFYSNIMNFFKTEIT
LANGGGSSGGSSGSETPGTSESATPESSGGSSGGSSTLNIEDEYRLHETS
KEPDVSLGSTWLSDFPQAWAETGGMGLAVRQAPLIIPLKATSTPVSIK
QYPMSQEARLGIKPHIQRLLDQGILVPCQSPWNTPLLPVKKPGTNDYR
PVQDLREVNKRVEDIHPTVPNPYNLLSGLPPSHQWYTVLDLKDAFFCL
RLHPTSQPLFAFEWRDPEMGISGQLTWTRLPQGFKNSPTLFNEALHRD
LADFRIQHPDLILLQYVDDLLLAATSELDCQQGTRALLQTLGNLGYRA
SAKKAQICQKQVKYLGYLLKEGQRWLTEARKETVMGQPTPKTPRQL
REFLGKAGFCRLFIPGFAEMAAPLYPLTKPGTLFNWGPDQQKAYQEIK
QALLTAPALGLPDLTKPFELFVDEKQGYAKGVLTQKLGPWRRPVAYL
SKKLDPVAAGWPPCLRMVAAIAVLTKDAGKLTMGQPLVILAPHAVE
ALVKQPPDRWLSNARMTHYQALLLDTDRVQFGPVVALNPATLLPLPE
EGLQHNCLDILAEAHGTRPDLTDQPLPDADHTWYTDGSSLLQEGQRK
AGAAVTTETEVIWAKALPAGTSAQRAELIALTQALKMAEGKKLNVYT
DSRYAFATAHIHGEIYRRRGWLTSEGKEIKNKDEILALLKALFLPKRLS
IIHCPGHQKGHSAEARGNRMADQAARKAAITETPDTSTLLIENSSPSGG
SSGGSSGSETPGTSESATPESSGGSSGGSEIRKRPLIETNGETGEIVWDK
GRDFATVRKVLSMPQVNIVKKTEVQTGGFSKESILPKRNSDKLIARKK
DWDPKKYGGFDSPTVAYSVLVVAKVEKGKSKKLKSVKELLGITIMER
SSFEKNPIDFLEAKGYKEVKKDLIIKLPKYSLFELENGRKRMLASAGEL
QKGNELALPSKYVNFLYLASHYEKLKGSPEDNEQKQLFVEQHKHYLD
EIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGG
D
Inlaid MMLV-RT in SpCas9 variant 2 (G1247/S1248; no NLS; RT with
flanking 32 AA linkers)
SEQ ID NO: 43
MDKKYSIGLDIGTNSVGWAVITDEYKVPSKKFKVLGNTDRHSIKKNLI
GALLFDSGETAEATRLKRTARRRYTRRKNRICYLQEIFSNEMAKVDDS
FFHRLEESFLVEEDKKHERHPIFGNIVDEVAYHEKYPTIYHLRKKLVDS
TDKADLRLIYLALAHMIKFRGHFLIEGDLNPDNSDVDKLFIQLVQTYN
QLFEENPINASGVDAKAILSARLSKSRRLENLIAQLPGEKKNGLFGNLI
ALSLGLTPNFKSNEDLAEDAKLQLSKDTYDDDLDNLLAQIGDQYADL
FLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGT
EELLVKLNREDLLRKQRTFDNGSIPHQIHLGELHAILRRQEDFYPFLKD
NREKIEKILTFRIPYYVGPLARGNSRFAWMTRKSEETITPWNFEEVVDK
GASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEI
SGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGK
TILDFLKSDGFANRNFMQLIHDDSLTFKEDIQKAQVSGQGDSLHEHIA
NLAGSPAIKKGILQTVKVVDELVKVMGRHKPENIVIEMARENQTTQK
GQKNSRERMKRIEEGIKELGSQILKEHPVENTQLQNEKLYLYYLQNGR
DMYVDQELDINRLSDYDVDAIVPQSFLKDDSIDNKVLIRSDKNRGKS
DNVPSEEVVKKMKNYWRQLLNAKLITQRKFDNLTKAERGGLSELDK
AGFIKRQLVETRQITKHVAQILDSRMNTKYDENDKLIREVKVITLKSKL
VSDFRKDFQFYKVREINNYHHAHDAYLNAVVGTALIKKYPKLESEFV
YGDYKVYDVRKMIAKSEQEIGKATAKYFFYSNIMNFFKTEITLANGEI
RKRPLIETNGETGEIVWDKGRDFATVRKVLSMPQVNIVKKTEVQTGG
FSKESILPKRNSDKLIARKKDWDPKKYGGFDSPTVAYSVLVVAKVEK
GKSKKLKSVKELLGITIMERSSFEKNPIDFLEAKGYKEVKKDLIIKLPK
YSLFELENGRKRMLASAGELQKGNELALPSKYVNFLYLASHYEKLKG
GGSSGGSSGSETPGTSESATPESSGGSSGGSSTLNIEDEYRLHETSKEPD
VSLGSTWLSDFPQAWAETGGMGLAVRQAPLIIPLKATSTPVSIKQYPM
SQEARLGIKPHIQRLLDQGILVPCQSPWNTPLLPVKKPGTNDYRPVQD
LREVNKRVEDIHPTVPNPYNLLSGLPPSHQWYTVLDLKDAFFCLRLHP
TSQPLFAFEWRDPEMGISGQLTWTRLPQGFKNSPTLFNEALHRDLADF
RIQHPDLILLQYVDDLLLAATSELDCQQGTRALLQTLGNLGYRASAKK
AQICQKQVKYLGYLLKEGQRWLTEARKETVMGQPTPKTPRQLREFLG
KAGFCRLFIPGFAEMAAPLYPLTKPGTLFNWGPDQQKAYQEIKQALLT
APALGLPDLTKPFELFVDEKQGYAKGVLTQKLGPWRRPVAYLSKKLD
PVAAGWPPCLRMVAAIAVLTKDAGKLTMGQPLVILAPHAVEALVKQ
PPDRWLSNARMTHYQALLLDTDRVQFGPVVALNPATLLPLPEEGLQH
NCLDILAEAHGTRPDLTDQPLPDADHTWYTDGSSLLQEGQRKAGAAV
TTETEVIWAKALPAGTSAQRAELIALTQALKMAEGKKLNVYTDSRYA
FATAHIHGEIYRRRGWLTSEGKEIKNKDEILALLKALFLPKRLSIIHCPG
HQKGHSAEARGNRMADQAARKAAITETPDTSTLLIENSSPSGGSSGGS
SGSETPGTSESATPESSGGSSGGSSPEDNEQKQLFVEQHKHYLDEIIEQI
SEFSKRVILADANLDKVLSAYNKHRDKPIREQAENIIHLFTLTNLGAPA
AFKYFDTTIDRKRYTSTKEVLDATLIHQSITGLYETRIDLSQLGGD

In some embodiments of the methods and compositions described herein, variants of any of the proteins or nucleic acids described herein can also be used that are at least 80%, 85%, 90%, 95%, 97%, 98%, or 99% identical to a sequence provided herein can also be used, so long as they retain desired functionality of the parental sequence. Residues that can be changed without destroying function can be identified, e.g., by aligning similar sequences and making conservative substitutions in non-conserved regions. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

### Expression Constructs

Expression constructs comprising sequences encoding components as described herein (Cas, RT, pegRNA, ngRNA, and/or sgNA, wherein the Cas and RT are in separate expression constructs or are expressed as separate proteins: the Cas can be encoded as a single protein or a split intein) can include viral vectors, including recombinant retroviruses, adenovirus, adeno-associated virus, lentivirus, and herpes simplex virus-1, or recombinant bacterial or eukaryotic plasmids.

Suitable expression constructs can include: a coding region; a promoter sequence, e.g., a promoter sequence that restricts expression to a selected cell type, a conditional promoter, or a strong general promoter; an enhancer sequence; untranslated regulatory sequences, e.g., a 5′untranslated region (UTR), a 3′UTR; a polyadenylation site; and/or an insulator sequence. Such sequences are known in the art, and the skilled artisan would be able to select suitable sequences. See, e.g., Current Protocols in Molecular Biology, Ausubel, F. M. et al. (eds.) Greene Publishing Associates, (1989). Sections 9.10-9.14; Vaneura (ed.), Transcriptional Regulation: Methods and Protocols (Methods in Molecular Biology (Book 809)) Humana Press; 2012 edition (2011) and other standard laboratory manuals. In some embodiments, the expression construct is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the α-fetoprotein promoter (Campes and Tilghman (1989) Genes Dev. 3:537-546).

A preferred approach for in vivo introduction of nucleic acid into a cell is by use of a viral vector containing a nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid. Viral vectors transfect cells directly; plasmid DNA can be delivered naked or with the help of, for example, cationic liposomes (lipofectamine) or derivatized (e.g., antibody conjugated), polylysine conjugates, gramacidin S, artificial viral envelopes or other such intracellular carriers, as well as direct injection of the nucleic acid construct (e.g., mRNA) or CaPO4 precipitation carried out in vivo.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381: Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 d1324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

Yet another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol. 158:97-129 (1992). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993).

In addition to viral transfer methods, such as those illustrated above, non-viral methods can also be employed to cause expression of a nucleic acid compound described herein (e.g., a nucleic acid encoding a component as described herein) in a cell or tissue, in vitro, ex vivo, or in vivo, e.g., in the tissue of a subject. Typically non-viral methods of gene transfer rely on the normal mechanisms used by mammalian cells for the uptake and intracellular transport of macromolecules. In some embodiments, non-viral gene delivery systems can rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al., J. Invest. Dermatol. 116(1): 131-135 (2001); Cohen et al., Gene Ther. 7(22): 1896-905 (2000); or Tam et al., Gene Ther. 7(21): 1867-74 (2000).

In some embodiments, an expression construct (or naked mRNA) is entrapped in liposomes bearing positive charges on their surface (e.g., lipofectins), which can be tagged with antibodies against cell surface antigens of the target tissue (Mizuno et al., No Shinkei Geka 20:547-551 (1992); PCT publication WO91/06309; Japanese patent application 1047381; and European patent publication EP-A-43075).

These constructs can be administered in any effective carrier, e.g., any formulation or composition capable of effectively delivering the sequence encoding the component to cells in vivo. For example, in clinical settings, the gene delivery systems for the therapeutic gene can be introduced into a subject by any of a number of methods, each of which is familiar in the art. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the recombinant gene is more limited, with introduction into the subject being quite localized. For example, the gene delivery vehicle can be introduced by catheter (see U.S. Pat. No. 5,328,470) or by stereotactic injection (e.g., Chen et al., PNAS USA 91: 3054-3057 (1994)).

The pharmaceutical preparation of the constructs can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

### Methods of Use

The present compositions can be used for prime editing of sequences in eukaryotic cells, e.g., mammalian (e.g., human or non-human mammals), avian, reptilian, yeast, and so on; prokaryotic cells (e.g., bacteria and archaea); and plant cells. In general, the methods include expressing in, or introducing into, the cells a Cas and an RT as described herein. The methods also include expressing in, or introducing into, the cells at least a pegRNA, as well as optionally an additional secondary nick mediated by a nicking gRNA (ngRNA) is introduced either up- or down-stream of the desired edit site and on the strand opposite the one nicked by the PE protein/pegRNA complex (as is done in PE3), and/or a ngRNA that binds only the edited DNA sequence (as is done in PE3b).

Prime editing methods are described in Scholefield et al., Gene Therapy 28:396-401 (2021); Anzalone et al., Nature 576:149-157 (2019); Hsu et al., Nature Communications 12:1034 (2021); WO/2020/191246; WO/2020/191249; WO/2020/191243; WO/2020/191241; WO/2020/191248; WO/2020/191245; WO/2020/191239; WO/2020/191171; WO/2020/191153; WO/2020/191234; WO/2020/191233; and WO/2020/191242, inter alia.

In addition, the variant RTs described herein can be used for transcribing RNA into DNA in vitro. These methods include contacting the RNA (i.e., template RNA to be transcribed) with an RT, wherein the RT comprises a truncated variant MMLV-RT as described herein, a variant MarathonRT protein as described herein, in a reaction mixture that also includes suitable buffers and sufficient nucleotides (e.g., dNTPs, optionally radiolabeled dNTPS or other dNTPs) to transcribe the DNA (as well as other factors necessary for the reaction to run), as well as other optional components such as RNAse inhibitors. For example, the variants can be used in RT-PCR reactions or for generating cDNA from mRNA. Also provided herein are kits comprising the variant RTs, buffers, and dNTPs, and optionally primers. e.g., random primers.

### EXAMPLES

The invention is further described in the following examples, which do not limit the scope of the invention described in the claims.

### Methods

The following methods and materials were used in the Examples set forth below.

### Molecular Cloning.

Prime editor (PE), Cas9 nuclease, reverse transcriptase (RT), and fusion constructs used in this study (Table 1) were cloned into a pCMV-T7 mammalian expression vector backbone obtained by AgeI-HF and NotI-HF (New England Biolabs. NEB) restriction digest of Addgene plasmid no. 112101 or 132775) as described below. All constructs that express PE2, SpCas9(H840A), MMLV-RT and its variants, XTEN linkers, and/or bipartite NLSs were cloned using Addgene plasmid no. 132775 as the PCR template. SaCas9-KKH based constructs were cloned using Addgene plasmid no. 70708 as a template. WT SaCas9 based constructs were cloned using Addgene plasmid no. 61594 as a template. Some constructs were cloned as P2A-eGFP fusions to obtain cotranslational expression of enhanced GFP (eGFP; P2A-eGFP generated using Addgene no. 112101 as template). DNA encoding alternative RTs were purchased from IDT as synthetic dsDNA products (IDT gblocks) with codon optimization for expression in human cells (GenScript GenSmart codon optimization tool). Gibson fragments with complementary overhangs were generated by PCR using Phusion high-fidelity DNA polymerase (NEB), which were then directly purified using paramagnetic beads²⁶ or purified after agarose gel electrophoresis and extraction using Qiaquick gel extraction kit (Qiagen). The purified DNA fragments were then assembled with a pCMV backbone at 50° C. for 1 h using Gibson mix²⁷ and used to transform chemically competent Escherichia coli XL1-Blue (Agilent). The prime editing gRNAs (pegRNAs) used in this study (Table 2) were cloned based on the protocol described by Anzalone et all. First, the oligos for the spacer, 5′ phosphorylated scaffold, and 3′ extension for each guide were annealed to form dsDNA fragments (95° C. for 5 min, then cooled to 10° C. at a rate of −5° C./min) with compatible overhangs for ligation to each other and to the BsaI-digested pUC19-based hU6-pegRNA-gg-acceptor entry vector (Addgene no. 132777). Subsequently, the vector backbone and the DNA duplexes were ligated using T4 ligase (NEB). Construction of SpCas9 and SaCas9 pegRNAs required different scaffolds. All SpCas9 pegRNAs (pre-extension) were of the form 5′-NNNNNNNNNNNNNNNNNNNNGTITTAGAGCTAGAAATAGCAAGTTAAAA TAAGGCTAGTCCGTTATCAACTTGAAAAAGTGGCACCGAGTCGGTGCNNN NNNNNNNNNNNNNNNNNTTTTTTT-3′ (SEQ ID NO: 44) (from BsaI digest of pU6-pegRNA-GG-acceptor, Addgene #132777). All SaCas9 pegRNAs (pre-extension) were of the form 5′-NNNNNNNNNNNNNNNNNNNN(20-22N spacer length)GTTTAGTACTCTGTAATGAAAATTACAGAATCTACTAAAACAAGG CAAAATGCCGTGTTTATCTCGTCAACTTGTTGGCGAGA-3′ (SEQ ID NO: 45; entry vector used=BsaI digest of pU6-pegRNA-GG-acceptor, Addgene #132777; SpCas9 scaffold replaced with SaCas9 scaffold via 5′ phosphorylated oligos with matching overhangs). Nicking gRNAs (ngRNAs) were generated in a similar fashion using only spacer oligos along with the BsmBI-digested pUC19-based hU6 gRNA entry vector BPK1520²⁸ (Addgene no. 65777) for SpCas9 ngRNAs and BPK2660⁴ (Addgene no. 70709) for SaCas9 ngRNAs. All SpCas9 PE3/PE3b nicking gRNAs were of the form 5′-NNNNNNNNNNNNNNNNNNNNGTTTTAGAGCTAGAAATAGCAAGTTAAAA TAAGGCTAGTCCGTTATCAACTTGAAAAAGTGGCACCGAGTCGGTGCTTT TT-3′ (SEQ ID NO: 46: from BsmbI digest of BPK1520, Addgene #65777). All SaCas9 PE3/PE3b nicking gRNAs were of the form 5′-NNNNNNNNNNNNNNNNNNNN(20-22N spacer length)GTTTAGTACTCTGTAATGAAAATTACAGAATCTACTAAAACAAGG CAAAATGCCGTGTTTATCTCGTCAACTTGTTGGCGAGA-3′ (SEQ ID NO: 47; from BsmbI digest of BPK2660, Addgene #70709). All the plasmids used in this study were purified using Qiagen Mini/Midi Plus kits.

Cell culture. We used STR-authenticated HEK293T cells (CRL-3216, ATCC) and U2OS cells (similar match to HTB-96: gain of no. 8 allele at the D5S818 locus), cultured in Dulbecco's modified Eagle medium supplemented with 10% FBS and 50 units/ml penicillin and 50 μg/ml streptomycin (all from Gibco). U2OS cells were supplemented with an additional 1% GlutaMAX (Gibco). Cells were grown at 37° C. with 5% CO₂ and passaged every 2-3 days when cells reached approximately 80% confluency. For experiments with iCell Cardiomyocytes (obtained from Cellular Dynamics/Fujifilm, item 11713), plating medium (Cellular Dynamics) was thawed overnight at 4° C. before thawing the cells according to the manufacturer's recommendations. After resuspension and counting, 2.5×10⁴ cells were seeded in 100 μL plating medium per well of a 96-well plate that had previously been coated with 0.1% gelatin for 4 hours. Maintenance medium (Cellular Dynamics) was thawed overnight at 4° C. 24 h before use, followed by equilibration at 37° C. Cells were carefully washed with maintenance medium 48 h post-seeding and plating medium was replaced with 90 μL maintenance medium per well, which was replaced every other day. Cells were maintained at 37° C. under 5% CO₂. Every 4 weeks, cell cultures were tested for mycoplasma contamination using the MycoAlert PLUS mycoplasma detection kit (Lonza) and all the results were negative for the duration of this study.

### Transfections and Nucleofections.

For transfections, HEK293T cells were seeded at 1.25×10⁴ cells in 92 mL growth medium/well in 96-well flat-bottom cell culture plates (Corning). After 18-24 h of growth, the cells were transfected with 43.3 ng of plasmid DNA in total (30 ng PE, 10 ng pegRNA, 3.3 ng ngRNA for fused (also referred to as intact) PE variants: 15 ng nCas9, 15 ng RT. 10 ng pegRNA, 3.3 ng ngRNA for split variants, using 0.3 μL of lipofection reagent TransIT-X2 (Mirus) and 9 μL of Opti-MEM (Gibco) per well. For off-target experiments, HEK293T cells were seeded into a 24-well plate flat-bottom format (Corning) (6.25×10⁴ cells/well). After 18-24 h of growth, the cells were transfected with 216.5 ng of plasmid DNA in total (150 ng PE, 50 ng pegRNA, 16.5 ng ngRNA for intact PE variants: 75 ng nCas9, 75 ng RT, 50 ng pegRNA, 16.5 ng ngRNA for split variants). For experiments with U2OS cells, 4×10⁶ cells were seeded into a 15-cm dish (Corning) in 25 ml growth medium. After 18-24 h of incubation, 2×10⁵ cells/sample were electroporated with 1083.3 ng of total plasmid DNA (800 ng PE, 200 ng pegRNA, 83.3 ng ngRNA for intact PE variants: 400 ng nCas9, 400 ng RT, 200 ng pegRNA, 83.3 ng ngRNA for split variants) using the SE cell Line Nucleofector X Kit (Lonza) according to the manufacturer's protocol. Subsequently, the electroporated cells were plated in 500 μL growth media in 24-well flat-bottom plates (Corning). iCell cardiomyocytes were transfected using Transit-LT1 transfection reagent³⁵ (Mirus) on days 5, 6, and 7 post-thawing, using 150 ng PE, 50 ng pegRNA, and 17 ng ngRNA for intact PE variants or 75 ng nCas9, 75 ng RT, 50 ng pegRNA, and 17 ng ngRNA for split PE variants as well as 9 μL Opti-MEM (Gibco) and 0.6 μL Transit-LT1 per well. Maintenance medium was replaced 3 h pre-transfection and 24 h post-transfection. Transfected and electroporated cells were incubated at 37° C. under 5% CO₂ for 72 h, followed by genomic DNA (gDNA) extraction.

### AAV Experiments.

AAVs were produced in HEK293T cells by PEI triple transfection of ΔF6 helper plasmid (Addgene no. 112867), AAV2/2 package plasmid (Addgene no. 104963), and an AAV2 ITR-flanked transgene containing plasmid. AAVs were purified and concentrated by sucrose density gradient ultracentrifugation to a final titer between 10¹² and 10¹³ genome copies/ml. The viruses were packaged at the MGH Vector Core Facility, Massachusetts General Hospital Neuroscience Center, Charlestown, MA. Transductions were carried out in 96-well format, where 10 μl of each of the two AAVs (or of one only for the negative control), encoding either nSpCas9 or MMLV-RTΔRH-P2A-eGFP and the two guide RNAs were applied to 1.5×10⁴ U2OS cells per well which were cultured in 50 μl of DMEM. One week post-transduction, cells were sorted for top ˜10-20% FITC mean fluorescence intensity and these cells were then seeded and cultured for another 72 hours before gDNA extraction.

### DNA Extraction.

After an initial wash step with 1×PBS, cells in 96-well format experiments were lysed with 43.5 mL gDNA lysis buffer (100 mM Tris-HCl (pH 8), 200 mM NaCl, 5 mM EDTA, 0.05% SDS), 1.25 mL 1 M DTT (Sigma), and 5.25 mL Proteinase K (800 U/ml, NEB) per well. Cells transfected or electroporated in a 24-well plate were lysed with the same components as listed but with 4× the amount, totaling 200 μL/well. Cells were lysed overnight in a shaker (HT Infors Multitron) at 500 rpm, at 55° C. and the gDNA was extracted with 2× paramagnetic beads as described previously²⁶. DNA bound to beads was washed with 70% ethanol three times using a Biomek FX^{p} Laboratory Automation Workstation (Beckman Coulter) and eluted in 35-75 mL 0.1× Buffer EB (Qiagen).

### Library Preparation for Targeted Amplicon Sequencing.

Concentrations of gDNA were determined using the Qubit4 fluorometer with the dsDNA HS Assay Kit (Thermo Fisher). Amplicons for sequencing were produced using a 2-PCR process to first amplify the specific target sequence and add Illumina adapter sequences (PCR1), and to subsequently add Illumina barcodes (PCR2). In PCR1, the target sequence was amplified from approximately 5-20 ng of gDNA using primers carrying Illumina-compatible adapter sequences with Phusion DNA polymerase (NEB) under the following reaction conditions: 98° C. for 2 min, followed by 30-35 cycles of 98° C. for 10 s, 68° C. for 12 s, and 72° C. for 12 s, and a final 72° C. extension for 10 min. The PCR products were purified with 0.7× paramagnetic beads, eluted in 30 μL EB buffer and quantified using the Quantifluor dsDNA quantification system (Promega) on a Synergy HT microplate reader (BioTek; set to 485/528 nm). In PCR2, unique Illumina-compatible barcodes were added to each PCR1 amplicon (based on NEBnext E7600 barcodes, as well as custom barcodes) using approximately 50-200 ng of the clean PCR1 product per sample (or per pool), and Phusion DNA polymerase (NEB). The reaction conditions were as follows: 98° C. for 2 min, 5-10 cycles of 98° C. for 10 s, 65° C. for 30 s, and 72° C. for 30 s, followed by a 72° C. extension for 10 min. In some cases, when PCR1 products stemmed from non-overlapping genomic sites, they were quantified using the Quantiflour system (Promega) and pooled before barcoding to allow sequencing of more samples per run. PCR2 products were cleaned with 0.7× paramagnetic beads, quantified with the Quantifluor system (Promega), and pooled to ensure equal representation of samples in the final library. The pooled PCR2 products were subjected to a final cleanup using 0.6× paramagnetic beads to reduce residual primers and primer-dimers. The resulting amplicons were sequenced using Illumina Miseq kits or Miseq micro kits (Miseq Reagent Kit v2; 300 cycles, 2×150 bp, paired-end). Demultiplexed sequencing data were downloaded in the form of FASTQ files via BaseSpace (Illumina).

### Deep Sequencing Analysis.

Sequencing files were analyzed using CRISPResso2²⁹ in HDR (homology directed repair) mode using standard parameters (unless otherwise indicated below). CRISPResso2 HDR categorizes sequencing reads into three distinct groups including ‘HDR’, ‘reference’ and ‘ambiguous’. Reads in the HDR group have a higher degree of sequence homology to the edited than to the unedited amplicons. The reads in the reference group have a higher degree of sequence homology to the unedited amplicons than to the edited amplicons. Reads in the ambiguous group are equally homologous to the edited and unedited amplicons (this can for example occur if the locus of the intended edit is deleted). The HDR group contained all reads harboring hallmarks of PE activity including pure PE containing only the intended edits and impure PE containing both the intended and unintended edits. To distinguish pure PE from impure PE, two editing windows were defined. One editing window spans from one bp before the predicted PE2 nicking location to one bp after the end of the DNA sequence that is homologous to the pegRNA RT template. The second HDR window spans from one bp before to one bp after the putative nicking site of the ngRNA. If apart from the intended edit, other mutations were detected within the editing window, reads were categorized as impure PE, otherwise as pure PE. The reference group contained all reads with neither the intended edit nor other mutations in the editing window. CRISPResso2 HDR categorizes reads without the intended edit but with additional mutations as ambiguous (if the locus of the intended edit was deleted) or as NHEJ (if the locus of the intended edit was intact but an edit was observed within the editing window). The reads of both groups (“ambiguous” and “NHEJ”) were interpreted as representing undesired PE byproducts. CRISPResso2 HDR was run with quality filtering (only reads with an average quality score>=30 were considered).

### Analysis of Editing Frequencies at Off-Target Sites.

Sequencing files were analyzed with CRISPResso2. An editing window was defined for every pegRNA which ranged from the first base before the putative Cas9 induced nick to one base after the end of the pegRNA RTT at the on-target site. The size of this editing window is defined as A. For every off-target candidate of a particular pegRNA, an editing window of size A was defined starting from the first base before the putative Cas9 nick. Sequencing reads with basepair insertions or deletions overlapping with the editing window were defined as edited; the remaining reads were defined as unedited. The fraction of edited reads is reported as the editing frequency.

### PyMOL Analysis.

The structure of the E. rectale RT (Marathon-RT; PDB 5HHL¹⁸) and of the GsI-IIC group II intron maturase RT (commercially available as TGIRT-III) complexed with an RNA template-DNA primer duplex (PDB 6AR1¹⁷) were downloaded from the PDB and visualized with PyMOL v.2.3.4 and 2.5 (Schrödinger). A structure prediction of full-length Marathon-RT was generated using Phyre 2²⁰ and was subsequently aligned with the structure of GsI-IIC RT in complex with an RNA-DNA duplex (PDB 6AR1) using the ‘align’ command (‘align structure1, structure2, object=alnobj’). All illustrations (FIG. 2E) were generated with PyMOL 2.5.

Statistics and data reporting. All bar graphs show the mean and error bars represent the standard deviation (s.d.). Error bars are shown when three independent replicates were performed (i.e. not in screening conditions, e.g. FIGS. 2A, C, F). All sequencing data were processed using CRISPResso 2.1.3 (Python 3.8). Microsoft Excel for Mac 16.19 (181109) was used to perform the unpaired, two-tailed t-tests (homoscedastic, i.e. assuming the two samples have equal or similar variance) that were used to calculate the p-values. GraphPad Prism 9.2.0 was used for final data analyses and generation of graphs. For the scatter plots in FIGS. 2C and 17A-1B, we used simple linear regression via GraphPad Prism 9.2.0. We did not predetermine sample sizes based on statistical methods. Investigators were not blinded to experimental conditions or assessment of experimental outcomes.

TABLE 1
List of constructs with nucleotide and amino
acid sequences (Sequences below in Table)

| | Difference | | |
|---|---|---|---|
| | from WT -or- | Nuc | AA |
| Construct | PMID | SI# | SI# |
| bpNLS-MMLV RT-4AA linker- | dual bpNLS | 48 | 49 |
| bpNLS | | | |
| bpNLS-MMLV RT(246AA | 246AA truncation from C- | 50 | 51 |
| truncation)-4AA linker-bpNLS | terminus (432-end), dual bpNLS | | |
| bpNLS-MMLV RT(23AA | 23AA truncation from N- | 52 | 53 |
| truncation)-4AA linker-bpNLS | terminus (1-23), dual bpNLS | | |
| bpNLS-MMLV RT(207AA | 207AA truncation from C- | 54 | 55 |
| truncation)-4AA linker-bpNLS | terminus (471-end), dual bpNLS | | |
| bpNLS-MMLV RT(316AA | 316AA truncation from C- | 56 | 57 |
| truncation)-4AA linker-bpNLS | terminus (362-end), dual bpNLS | | |
| bpNLS-MMLV RT(181AA | 181AA truncation from C- | 58 | 59 |
| truncation)-4AA linker- | terminus (497-end), dual bpNLS | | |
| bpNLS = MMLV-RT(dRH) | | | |
| bpNLS-MMLV RT(23AA + 181AA | 23AA truncation from N- | 60 | 61 |
| truncation)-4AA linker-bpNLS | terminus (1-23) and 181AA | | |
| | truncation from C-terminus | | |
| | (497-end), dual bpNLS | | |
| bpNLS-MMLV RT(dRH)-4AA- | 181AA truncation from C- | 62 | 63 |
| bpNLS-P2A-eGFP2394 | terminus (497-end), dual bpNLS | | |
| bpNLS-nCas9(H840A)-P2A- | co-translational expresssion of | 64 | 65 |
| MMLV RT(dRH)-4 AA linker- | nCas9(H840A) & MMLV | | |
| bpNLS | RT(dRH) | | |
| bpNLS-HFV RT-4AA linker- | dual bpNLS | 66 | 67 |
| bpNLS | | | |
| bpNLS-HERV-Kcon RT-4AA | PMID 15163704, dual bpNLS | 68 | 69 |
| linker-bpNLS | | | |
| bpNLS-LtrA RT-4AA linker- | PMID 17257061, dual bpNLS | 70 | 71 |
| bpNLS | | | |
| bpNLS-TeI4c RT-4AA linker- | PMID 29153391, dual bpNLS | 72 | 73 |
| bpNLS | | | |
| bpNLS-Ma-int5 RT-4AA linker- | PMID 23697550, dual bpNLS | 74 | 75 |
| bpNLS | | | |
| bpNLS-GsI-IIc RT-4AA linker- | PMID 15574519, dual bpNLS | 76 | 77 |
| bpNLS | | | |
| bpNLS-Marathon RT-4AA linker- | PMID 29153391, dual bpNLS | 78 | 79 |
| bpNLS | | | |
| bpNLS-Marathon(D14R-N26R- | PMID 29109157, D14R-N26R- | 80 | 81 |
| D74R-N116K-N197R) RT-4AA | D74R-N116K-N197R, dual | | |
| linker-bpNLS | bpNLS | | |
| bpNLS-nCas9(H840A)-XTEN- | P2A-eGFP at C-terminus | 82 | 83 |
| MMLV RT-4AA linker-bpNLS- | | | |
| P2A-eGFP | | | |
| bpNLS-MMLV RT-XTEN- | N-terminal fusion of MMLV-RT | 84 | 85 |
| nCas9(H840A)-4AA linker-P2A- | pentamutant | | |
| eGFP | | | |
| bpNLS-nCas9(H840A)pt. 1-32AA | MMLV-RT (pentamutant) inlaid | 86 | 87 |
| linker-MMLV RT-32AA linker- | at G1247 | | |
| nCas9(H840A)pt. 2-4 AA linker- | | | |
| bpNLS-P2A-eGFP -- MMLV-RT | | | |
| inlaid at G1247 | | | |
| bpNLS-nCas9(H840A)-XTEN-4 | nCas9-only for co-expression | 88 | 89 |
| AA linker-bpNLS-P2A-eGFP | with untethered RT (Split-PE) | | |
| bpNLS-MMLV RT-4 AA linker- | MMLV-RT (pentamutant) only | 90 | 91 |
| bpNLS-P2A-eGFP | for co-expression with | | |
| | untethered RT (Split-PE), dual | | |
| | bpNLS | | |
| bpNLS-nCas9(H840A)pt. 1-32AA | MMLV-RT (pentamutant) inlaid | 92 | 93 |
| linker-MMLV RT-32AA linker- | at G1055 | | |
| nCas9(H840A)pt. 2-4 AA linker- | | | |
| bpNLS-P2A-eGFP -- MMLV-RT | | | |
| inlaid at G1055 | | | |
| bpNLS-nSaCas9(N580A)KKH- | Use of nSaCas9(N580A)KKH in | 94 | 95 |
| XTEN-MMLV RT-4AA linker- | PE2 architecture | | |
| bpNLS-P2A-eGFP | | | |
| bpNLS-nSaCas9(N580A)KKH- | Combined use of | 96 | 97 |
| XTEN-MMLV RT(dRH)-4AA | nSaCas9(N580A)KKH and | | |
| linker-bpNLS-P2A-cGFP | MMLV-RT(dRH) in PE2 | | |
| | architecture | | |
| bpNLS-nSaCas9(N580A)KKH- | Use of nSaCas9(N580A)KKH in | 98 | 99 |
| XTEN-4AA linker-bpNLS-P2A- | Split-PE architecture (with | | |
| eGFP | untethered, separately expressed | | |
| | RT domain), dual bpNLS | | |
| bpNLS-nSaCas9(N580A)-XTEN- | Use of nSaCas9(N580A) in | 100 | 101 |
| 4AA linker-bpNLS-P2A-eGFP | Split-PE architecture (with | | |
| | untethered, separately expressed | | |
| | RT domain), dual bpNLS | | |
| bpNLS-nCas9(H840A)-XTEN- | Fusion of delta RNAseH | 102 | 103 |
| MMLV RT(dRH)-4 AA linker- | MMLV-RT to nCas9 (not Split- | | |
| bpNLS-P2A-eGFP | PE | | |
| nSpCas9(H840A) | Split-PE construct 1 (nickase- | 104 | 105 |
| | only) for expression and delivery | | |
| | with dual-AAV vectors | | |
| pegRNA-pH1-ngRNA-pEFS- | Split-PE construct 2 | 106 | 107 |
| bpNLS-MMLVRT(dRH)-bpNLS- | (pegRNA/ngRNA/RT) for | | |
| 2A-eGFP | expression and delivery with | | |
| | dual-AAV vectors | | |
| bpNLS-nCas9(H840A)-XTEN- | Marathon-RT pentamutant | 108 | 109 |
| Marathon RT(D14R-N26R-D74R- | (D14R-N26R-D74R-N116K- | | |
| N116K-N197R)-4AA linker- | N197R) fused to nSpCas9 (not | | |
| bpNLS-P2A-eGFP | Split-PE) | | |
| bpNLS-Marathon(D14R-D74R- | Marathon-RT tetramutant. | 110 | 111 |
| N116K-N197R) RT -4AA linker- | (D14R-D74R-N116K-N197R) | | |
| bpNLS | for use in Split-PE (untethered | | |
| | RT) | | |
| bpNLS-nCas9(N)-N intein | Intein-based split of PE2, PMID | 112 | 113 |
| | 33837189 | | |
| C intein-nCas9(C)-XTEN-MMLV | Intein-based split of PE2, PMID | 114 | 115 |
| RT-bpNLS | 33837189 | | |
| bpNLS-nCas9(H840A)-XTEN- | WT Marathon-RT fused to | 116 | 117 |
| Marathon RT-4AA linker-bpNLS- | nSpCas9 (not Split-PE) | | |
| P2A-eGFP | | | |
| bpNLS-nCas9(H840A)-XTEN- | Marathon-RT tetramutant | 118 | 119 |
| Marathon RT(D14R-D74R- | (D14R-D74R-N116K-N197R) | | |
| N116K-N197R)-4AA linker- | fused to nSpCas9 (not Split-PE) | | |
| bpNLS-P2A-eGFP | | | |

SI#, SEQ ID NO:
All plasmids are in a CMV backbone
All constructs are suitable for mammalian expression. Growth in bacteria: 37° C., resistance: Ampicillin
Unless otherwise noted, MMLV-RT constructs described herein are based on the pentamutant construct D200N/L603W/T330P/T306K/W313F.

### Example 1. Split CRISPR Prime Editors with Untethered Reverse Transcriptase Retain High Efficiencies in Human Cells

In the course of attempting to modify the architecture of the PE2 protein, it was inadvertently discovered that the pentamutant MMLV-RT is separable from nSpCas9. In initial experiments, alternative configurations of the components of PE2, including fusion of MMLV-RT to the N-terminus of nSpCas9 and certain inlaid fusions of MMLV-RT within the Cas9 nickase³, showed activity that was comparable or only moderately reduced relative to the original PE2 fusion when tested with 11 pegRNA/ngRNA combinations in HEK293T cells (FIGS. 1E-J). In addition, the frequencies of unwanted impure prime edit alleles (those with the desired edit together with an additional mutation) and byproduct alleles (indel mutations and/or substitutions) were observed with the 11 pegRNA/ngRNA pairs, and these alternative PE2 architectures did not appear to differ from those observed with PE2. These unexpected findings suggested that the pentamutant MMLV-RT, rather than functioning in cis on the same protein molecule with the nSpCas9 protein, might be acting in trans from another PE2 molecule not tethered to the target site. This in turn suggested that a split PE2 architecture (with the nSpCas9 and the pentamutant MMLV-RT expressed as wholly separate proteins from different plasmids) might also function comparably to intact PE2 protein. Indeed, we found that a Split-PE2 architecture was comparably efficient to the original intact PE2 when tested with the same 11 pegRNA/ngRNA pairs in HEK293T cells (FIGS. 1E-C, 1K-N). We tested inlaid MMLV-RT fusions, N-terminal RT fusions, and N-terminal and inlaid fusions of the truncated MMLV-RT delta RNAse H (dRH) variant and the d23_dRH double truncation variant side-by-side with PE2 (C-terminal fusion) and saw robust prime editing in human cells (FIGS. 1H-1N). We also tested whether a split version of another prime editor based on a Staphylococcus aureus Cas9 KKH PAM recognition variant nickase (nSaCas9-KKH)⁴ might also function comparably to its intact counterpart (FIG. 2G) and again found this to be true with six different pegRNA/ngRNA pairs targeting various endogenous gene sites in human HEK293T cells (FIG. 2G).

In addition, the frequencies of impure prime edits (IPEs—alleles with the desired edit together with an additional mutation) and byproducts (alleles with indels and/or substitutions but not the desired edit) we observed with the 11 pegRNA/ngRNA pairs and these alternative PE2 architectures did not appear to differ from those observed with PE2. (Note that for pegRNAs designed to introduce insertion and deletion edits, it is not always possible to distinguish IPE and byproduct alleles; in these cases, we group IPE and byproduct frequencies together and show them as combined outcome frequencies as we have done previously)²³.

These unexpected findings suggested to us that MMLV-RT, rather than functioning in cis on the same protein molecule with the nSpCas9 protein, might be acting in trans from another PE2 molecule not tethered to the target site. This in turn suggested that a split PE2 architecture (with the nSpCas9 and the MMLV-RT expressed as wholly separate proteins from different plasmids) might also function comparably to intact PE2 protein. Indeed, we found that a Split-PE2 architecture was comparably efficient to the original intact PE2 when tested with the same 11 pegRNA/ngRNA pairs in HEK293T cells (FIGS. 1E-G, 1M). In addition, we observed similar results in U2OS cells with Split-PE2 showing comparable or higher activities than intact PE2 with seven out of eight pegRNA/ngRNA pairs we tested (FIG. 1O). We also tested whether a split version of another prime editor based on a Staphylococcus aureus Cas9 KKH PAM recognition variant nickase (nSaCas9-KKH)⁴ might function comparably to its intact counterpart (FIG. 1N), and again found this to be true with six different pegRNA/ngRNA pairs targeting various endogenous gene sites in human HEK293T cells (FIG. 1N).

We next explored whether the splitting of PE2 into separated RT and nickase components might alter the off-target effects of prime editing. To do this, we assessed editing frequencies at 18 genomic sites using six pegRNA/ngRNA combinations. These genomic sites had previously been found to exhibit off-target editing with either intact PE2 and/or SpCas9 nuclease in human cells ((FIG. 1Q)^{1, 36, 37}. In our experiments, intact PE2 and Split-PE2 showed comparable on-target editing efficiencies with all six pegRNA/ngRNA combinations. We also observed comparable editing frequencies with intact PE2 and Split-PE2 at an off-target site that had been previously reported for two different pegRNA/ngRNA combinations at HEK site 4 (FIG. 1Q)¹. Importantly, we did not observe any evidence of new editing with Split-PE2 at any of the 17 other potential off-target sites that previously did not show evidence of editing with intact PE2 (FIG. 1Q).

An important implication of our findings with split PE proteins is that alternative RT enzymes (or CRISPR-Cas nickases) could potentially be rapidly tested without the need to optimize linker lengths or relative positions within a fusion protein. To test this, we tested six truncation mutants of the MMLV-RT pentamutant variants in the Split-PE2 configuration with three different pegRNA/ngRNA pairs targeting different endogenous human gene target sites (FIG. 2A). This included a previously described N-terminal truncation variant (truncation 2, lacking 23 residues)^{5, 6} as well as C-terminal truncation variants that included truncations of the connection (truncations 1, 3, and 4) and/or RNAse H domains (truncation 5)⁶⁻⁹.

| | | |
|---|---|---|
| Full length WT/pentamutant | 677AA | — |
| Truncation 1 | 431AA | delta 432-677 |
| Truncation 2 | 654 AA | delta 1-23 |
| Truncation 3 | 470AA | delta 471-677 |
| Truncation 4 | 361AA | delta 362-677 |
| Truncation 5 | 496AA | delta 497-677 |
| Truncation 6 | 473AA | delta 1-23 + 497-677 |

From these experiments, we identified a reduced-size MMLV-RT pentamutant variant (truncation 5) lacking the RNase H domain (MMLV-RTrRH) with activity equivalent to Split-PE2 (with full-length MMLV-RT pentamutant) (FIG. 2A, 3A). This truncated RT is 543 base pairs (bp) or 26.7% smaller than the parental MMLV-RT. To further assess the activity of this pentamutant (actually now a tetramutant, as AA603 is in the deleted region) MMLV-RTΔRH truncation, we tested it with 11 pegRNA/ngRNA pairs and found it functioned as efficiently as or better than full-length MMLV-RT pentamutant in the Split-PE2 configuration at 10 out of 11 sites in HEK293T cells (FIG. 2B, 3B). This truncated RT is encoded by 1488 bps and is therefore 26.7% smaller than the parental MMLV-RT. A recent study published by others while this work was in progress has also described a PE variant with a MMLV-RT truncation of the RNase H domain³⁹.

To further assess the activity of the MMLV-RTΔRH truncation, we tested it with eight additional pegRNA/ngRNA pairs and found it functioned as efficiently or better than full-length MMLV-RT in the Split-PE2 configuration with 10 out of 11 pegRNA/ngRNA pairs in HEK293T cells (FIGS. 2B, 3B). We obtained similar results in U2OS cells, with Split-PE2 using truncated MMLV-RTΔRH performing comparably to or better than Split-PE2 using the full-length MMLV-RT for seven out of the eight pegRNA/ngRNA pairs we tested (FIG. 1O).

We also observed comparable activities when the truncated MMLV-RTΔRH was expressed as a cleavable P2A translational fusion with the nSpCas9 from a single plasmid (and promoter) with the same 11 pegRNA/ngRNA pairs in HEK293T cells (FIG. 3C). We tested whether the MMLV-RTΔRH truncation could mediate prime editing with different nickases and found it worked as efficiently as full-length MMLV-RT pentamutant when co-expressed separately with nSaCas9, the nSaCas9-KKH variant, as a fusion with nSaCas9-KKH (FIGS. 4A and B), or inlaid into the nSpCas9 (FIGS. 15A-D). Finally, to test the MMLV-RTΔRH in a more disease-relevant, non-cancer cell line, we transfected human induced pluripotent stem cell (hiPSC)-derived cardiomyocytes with constructs expressing intact and Split-PE prime editor architectures using MMLV-RTΔRH together with four pegRNA/ngRNA combinations. We observed prime editing at all four sites with both intact and split PE2ΔRH (range of mean PPE frequencies across all four sites of 1.4 to 16.7%) (FIG. 1P). At all 4 sites in hiPSC-derived cardiomyocytes, the editing activities of intact and split PE2-ΔRH variants were also comparable as expected (FIG. 1P).

We additionally leveraged the simplified screening enabled by the split PE framework to test a set of seven different RT enzymes, each smaller in size than the MMLV-RT pentamutant. The coding sequences for these enzymes ranged in length from 1242 to 1827 bps, all providing reduced size alternatives to the 2031 bp MMLV-RT pentamutant (FIGS. 2C-2D; FIGS. 5A-C). Two of the seven RTs we tested were of viral (human foamy virus; HFV)^{10, 11} or human endogenous retroviral (HERV)¹² origin and the remaining five were group II intron RT domains (FIG. 2C)¹³⁻¹⁹. Testing of these RTs co-expressed with nSpCas9 and using three different pegRNA/ngRNA pairs revealed low prime editing frequencies in human HEK293T cells (FIG. 2C). The best performing RTs among the seven we tested were the HERV-K_{con} RT (˜1.2-3.5%) and the bacterial group II intron RTs GsI-IIC and Marathon (˜0.7-2.8%). Because of its small size and consistent activity across the three different pegRNA/ngRNA pairs tested, we selected the Marathon-RT (a maturase RT from Eubacterium rectale that is also commonly used for in vitro laboratory applications¹⁹) to carry forward for additional optimization.

To further improve the activity of Marathon-RT for prime editing, we created a series of rationally designed mutants and tested each of these with co-expressed nSpCas9 in human cells. To guide the choice of the mutations we created, we initially used Phyre2²⁰ to generate a predicted structural model of Marathon-RT and also used published high-resolution structures of Marathon-RT in isolation (PDB 5HHL¹⁸) and of the homologous GsI-IIC group II intron maturase RT (commercially available as TGIRT-III) complexed with an RNA template-DNA primer duplex (PDB 6AR1¹⁷) (FIG. 2E; Methods). By aligning our Marathon-RT structure prediction with the structure of GsI-IIC RT in complex with the RNA-DNA duplex, we identified 15 negatively charged or polar uncharged amino acid residues in Marathon-RT that were predicted to lie within the modeled DNA/RNA binding pocket of the enzyme (FIG. 2E). We hypothesized that changing each of these 15 positions to positively charged residues might potentially increase binding of the RT domain to the pegRNA and/or the nicked DNA exposed in the R-loop generated by a nickase Cas9. Based on this reasoning, we screened 30 different Marathon-RT variants harboring mutations at each of these positions with nSpCas9 and identified 15 that showed increased prime editing efficiencies relative to wild-type Marathon-RT when co-expressed with three different pegRNA/ngRNA pairs in HEK293T cells (FIGS. 6A-C). We also tested 18 additional Marathon-RT variants harboring various combinations of the seven most promising mutations (again with nSpCas9 and three pegRNA/ngRNA pairs) in HEK293T cells and several of these variants showed further improved activity. Notably, one Marathon-RT variant harboring five amino acid substitutions (D14R-N26R-D74R-N116K-N197R) showed 5.2- to 7.9-fold (mean of 6.1-fold) higher editing activity relative to the original Marathon-RT and achieved absolute prime editing frequencies ranging from ˜10-15% (see Table C, above; FIG. 2F and FIGS. 6A-C). Furthermore, we show that we could obtain efficient prime editing in human HEK293T cells when Marathon-RT and variants thereof were fused directly to the C-terminus of nSpCas9 (FIGS. 14A-G). Using this approach with e.g. Marathon tetra- and pentamutants editing frequencies of up to 29.6% were obtained, which corresponded to fold changes (compared to WT Marathon-RT) of up to 4.1.

To further validate our findings, we tested MMLV-RTΔRH and Marathon-RT in both intact and split PE configurations with 11 pegRNA/ngRNA combinations. These experiments in HEK293T cells showed that intact and split PEs with MMLV-RTΔRH exhibited comparable editing between intact and split architectures at 5 out of 11 sites, and somewhat reduced editing with the split configuration at the remaining six sites (FIG. 16). Overall, the intact and split PE2ΔRH editors showed comparable PPE frequencies ranging from 7.4-53% and 2.3-46.6%, respectively (FIG. 17A). For intact and split PE architectures made with the engineered tetramutant and pentamutant Marathon-RTs, the split versions outperformed the intact ones at 5 out of 11 sites (tetramutant) and 9 out of 11 sites (pentamutant), respectively, with PPE frequencies ranging from 0.4-26.2% (tetramutant, split) and 0.4-22.7% (pentamutant, split) (FIG. 16). The relative efficiencies of each of our Split-PE architectures using the MMLV-RTΔRH and pentamutant Marathon-RT differed substantially across the 11 different pegRNA/ngRNA pairs tested (FIGS. 16 and 17B), but we did not observe any obvious correlations between activities observed and the various lengths of the PBS and RTT regions of the pegRNAs tested.

Finally, we sought to compare our most active Split-PE2 architecture (using MMLV-RTΔRH) with an alternative split-intein PE2 protein that was published during the course of our experiments⁴⁰. As noted above, the large size of the intact PE2 protein precludes its delivery using viral vectors such as adeno-associated virus (AAV) or lentiviral vectors. However, it has been shown that PE2 can be divided into two parts in the middle of the SpCas9 nickase, and then reconstituted into intact functional PE2 if trans splicing inteins are placed at the location of the split (FIG. 18A)²⁶. The components of this split-intein PE2 can be delivered into cells in vivo using dual AAV vectors to mediate prime editing events⁴⁰. To compare this system with ours, we transfected HEK293T cells with plasmids encoding 11 pegRNA/ngRNA combinations and either our most efficient minimized Split-PE architecture (Split-PE2ΔRH) or the previously described split-intein PE2 architecture. For all 11 sites, we observed higher PPE frequencies with Split-PE2ΔRH compared with the split-intein PE2 (FIG. 18B), perhaps at least partly reflecting the additional requirement for a bimolecular fusion reaction necessary to generate functional PE2 in the latter system. We additionally tested whether our split prime editor system could be delivered using two AAV vectors. For this proof-of-concept experiment, we encoded the entire SpCas9 nickase in one AAV vector and the pegRNA/ngRNA combination for HEK site 3 (CTT insertion) and the MMLV-RTΔRH-P2A-eGFP construct in the other (FIG. 18C). Following sorting for GFP-positive cells (Methods), delivery of both vectors to U2OS cells yielded a mean PPE frequency of nearly 4% while delivery of only the pegRNA/ngRNA/RT vector did not yield detectable PPEs (FIG. 18D). This experiment establishes the feasibility of using AAV vectors to deliver our Split-PE2 components even without extensive optimization of experimental parameters such as number and ratios of viral particles.

EXEMPLARY SEQUENCES

SEQ ID NO: 1
>tr|E2GM63|E2GM63_GEOSE Trt OS = Geobacillus stearothermophilus OX = 1422
GN = trt PE = 1 SV = 1
MALLERILARDNLITALKRVEANQGAPGIDGVSTDQLRDYIRAHWSTIHAQLLAGTYRPAPVRRVEIPK
PGGGTRQLGIPTVVDRLIQQAILQELTPIFDPDFSSSSFGFRPGRNAHDAVRQAQGYIQEGYRYVVDMD
LEKFFDRVNHDILMSRVARKVKDKRVLKLIRAYLQAGVMIEGVKVQTEEGTPQGGPLSPLLANILLDDL
DKELEKRGLKFCRYADDCNIYVKSLRAGQRVKQSIQRFLEKTLKLKVNEEKSAVDRPWKRAFLGFSFTP
ERKARIRLAPRSIQRLKQRIRQLTNPNWSISMPERIHRVNQYVMGWIGYFRLVETPSVLQTIEGWIRRR
LRLCQWLQWKRVRTRIRELRALGLKETAVMEIANTRKGAWRTTKTPQLHQALGKTYWTAQGLKSLTQRY
FELRQG
SEQ ID NO: 2
>AAB06503.1 putative maturase (plasmid) [Lactococcus lactis subsp.
lactis]
MKPTMAILERISKNSQENIDEVFTRLYRYLLRPDIYYVAYQNLYSNKGASTKGILDDTADGFSEEKIKK
IIQSLKDGTYYPQPVRRMYIAKKNSKKMRPLGIPTFTDKLIQEAVRIILESIYEPVFEDVSHGFRPQRS
CHTALKTIKREFGGARWFVEGDIKGCFDNIDHVTLIGLINLKIKDMKMSQLIYKFLKAGYLENWQYHKT
YSGTPQGGILSPLLANIYLHELDKFVLQLKMKFDRESPERITPEYRELHNEIKRISHRLKKLEGEEKAK
VLLEYQEKRKRLPTLPCTSQTNKVLKYVRYADDFIISVKGSKEDCQWIKEQLKLFIHNKLKMELSEEKT
LITHSSQPARFLGYDIRVRRSGTIKRSGKVKKRTINGSVELLIPLQDKIRQFIFDKKIAIQKKDSSWFP
VHRKYLIRSTDLEIITIYNSELRGICNYYGLASNFNQLNYFAYLMEYSCLKTIASKHKGTLSKTISMFK
DGSGSWGIPYEIKQGKQRRYFANFSECKSPYQFTDEISQAPVLYGYARNTLENRLKAKCCELCGTSDEN
TSYEIHHVNKVKNLKGKEKWEMAMIAKQRKTLVVCFHCHRHVIHKHK
SEQ ID NO: 3
>BAC08171.1 reverse transcriptase [Thermosynechococcus elongatus BP-1]
METRQMAVEQTTGAVTNQTETSWHSIDWAKANREVKRLQVRIAKAVKEGRWGKVKALQWLLTHSFYGKA
LAVKRVIDNSGSKTPGVDGITWSTQEQKAQAIKSLRRRGYKPQPLRRVYIPKANGKQRPLGIPTMKDRA
MQALYALALEPVAETTADRNSYGFRRGRCIADAATQCHITLAKTDRAQYVLDADIAGCFDNISHEWLLA
NIPLDKRILRKWLKSGFVWKQQLFPIHAGTPQGGVISPMLANMTLDGMEELLNKFPRAHKVKLIRYADD
FVVTGETKEVLYIAGAVIQAFLKERGLTLSKEKTKIVHIEEGFDFLGWNIRKYDGKLLIKPAKKNVKAF
LKKIRDTLRELRTAPQEIVIDTLNPIIRGWTNYHKNQASKETFVGVDHLIWQKLWRWARRRHPSKSVRW
VKSKYFIQIGNRKWMFGIWTKDKNGDPWAKHLIKASEIRIQRRGKIKADANPFLPEWAEYFEQRKKLKE
APAQYRRTRRELWKKQGGICPVCGGEIEQDMLTETHHILPKHKGGTDDLDNLVLIHTNCHKQVHNRDGQ
HSRFLLKEGL
SEQ ID NO: 4
>WP_010967953.1 group II intron reverse transcriptase/maturase
[Sinorhizobium meliloti]
MTSESTTDKPFRIEKRRVYEAYKAVKANRGAAGVDGQTLEIFEKDLAANLYKIWNRMSSGTYFPPPVRA
VSIPKKAGGERVLGVPTVSDRIAQMVVKQMIEPDLDSLFLPDSYGYRPGKSALDAVGVTRQRCWKYDWV
LEFDIKGLFDNLPHDLLLKAVRKDVKCNWALLYIERWLTAPMEKNGEVIERSRGTPQGGVVSPILANLF
LHYAFDLWMTRTHPDLPWCRYADDGLVHCQSEQQAEALKVELSSRLAACGLQMHPTKTKIVYCKDQRRR
EAYPNVTFDFLGYQFRPRRVANTQWDEFFCGYTPAVSPTALKSMRATIKSLNIPRQTPGTLAEIAKQLN
PLLRGWIAYYGRYSRSALSTLADYVNQKLRAWIRRKFKRFQSHKTRASLFLRKLARENPGLFVHWKAFG
TNTFT
SEQ ID NO: 5
>AAM07961.1 reverse transcriptase [Methanosarcina acetivorans C2A]
MDETKPYEISKDIVQEAFQRVKANKGAAGVDDENIAAFESDLTNNLYKIWNRMSSGCYFPPSVKAIEIP
KKSGGTRILGIPTVLDRVAQMVTKIYLEPQLEPLFHPDSYGYRPGKSAADALAATRKRCWRYNWLLEFD
IKGLFDNINHDLLMKQVSMHTDKPWIILYIQRWLKAPFQMADGTVNERTKGTPQGGVVSPLLANLFLHY
AFDQWMDSHHRYNPFERYADDSVIHCRSREEAERLWIELDKRLSEFGLELHPSKTRIVYCKDDDRQGDY
PETKFDFLGYTFRPRRSKNKYGKHFINFTPAVSNTAKKSMQQEIHDWRMHLKPDKTLEDLSHMFNPILR
GWVNYYGLFYKSELYCVLKHMNRVLTRWAQRKYKKLAGHKRRARYWLGKIARRDPKLFVHWQMGIFPEA
G
SEQ ID NO: 6
>AEC33268.1 group IIC intron maturase [Enterobacter cloacae]
MRPLPQAVDEIQHHEVQNQPPRNPTSWMAQVLARDNLIRALNQVKRNKGAAGVDGMTVERLSDYLKQHW
PALKEQLETGNYQPEAVKRVEIPKADGRKRKLGIPTVLDRFIQQAIAQVLSQHWESQFHNNSYGFRPMR
SAHQAVSYAKALLLSGKGWVVDLDLDAFFDRVNHDRLMSKLRAQIQDPTLLKLIQRYLKANIDHNGKQE
ACREGVPQGGPLSPLLANIVLNELDWELERRGHSFARYADDCQIYTSSKRAGERIKQSIERYIETRERL
KVNKAKSAVARPWERSFLGFTFSRRKGNRLKVTDKALDRLKDKLRELTRRTRGHNIGSVIADIRKALLG
WKAYFGIAEVQSQLRDTDKWLRRKLRCYIWKQWGSKGYRMLRKAGVDRFLAWNTAKSAHGPWRLSKSPA
LYIALPNRYFTNMGLPTIAA
SEQ ID NO: 7
>NP_350100.1 Reverse transcriptase/maturase [Clostridium
acetobutylicum ATCC 824]
MKNSKEMQKLQTTSYKEGWSCEIRVELQNSTRAHSISTAFDRRKDDGKLYEINLLERILDRQNMNLAYK
RVKSNKGSHGVDGMKVDELLQYLKQNGKTLIASIFNGKYCPKAVRRVEIPKPDGGIRLLGIPTVVDRTI
QQAISQVLTPIFEKTFSENSYGFRPKRSAKQAIKKAKEYMEEGYKWVVDIDLAKYFDTVNHDKLMALVA
RKIKDKRVLKLIRLYLQSGVMINGVVSETERGCPQGGPLSPLLSNIMLTELDRELEKRGHKFCRYADDN
NVYVRSKKAGDRVMRSITRFIENKLKLKVNKEKSAVDRPWRRKFLGFTFYQWYGKIGIRVHEKSVKKFK
AKIKAITARSNALNIENRIIKLRQCIIGWINYFGIAEMTKLAKKLDEWTRRRLRMCYWKQWKKVKTKYD
NLRKFGINNSKAWEFANTRKSYWRIANSPILSTTLINSYLEKIGYTSIFKRYKQVH
SEQ ID NO: 8
>BAA90841. 1 unnamed protein product [Bacillus halodurans]
MLERILSRENLIQALERVEKNKGSYGVDEMDVKSLRLHLHENWTSIRNEIIEGSYFPKPVRRVEIPKPN
GGVRKLGIPTVMDRFLQQAIAQILTQLYDPTFSERSFGFRPHRRGHNAVRQAKQWMKEGYRWVVDIDLE
KFFDKVNHDRLMRKLSSRIQDPRVLQLIRRYLQTGVMERGLVSPNTEGTPQGGPLSPLLSNIVLDELDN
ELEKRGLKFVRYADDCNIYVRSKRAGLRIMESVTSFIENRLKLKVNREKSAVDRPWNRKFLGFSFTRGK
DPKMRVSKESVKRLKQRIRELTSRRHSMKMSDRLRRLNRYLIGWLGYYQLVDTPSILAQIDAWIRRRLR
MIRWKEWKTTSARQKNLVRLGIKKAKAWQWANSRKGYWRVAHSPIMDYALNSEYWKGQGLMSLAERYQT
RRWT
SEQ ID NO: 9
>AAB68949.1 maturase-related protein [Pseudomonas alcaligenes]
MPPVGVAVSLVTVMQKFPTAETVIPNPGQKPRVMPDSAKVPAASATWTNAEPDTLMERVLAPANLRRAY
QRVVSNKGAPGADGMTVADLAGYVKQYWPTLKARLLAGEYHPQAVRAVEIPKPQGGTRQLGIPSVVDRL
IQQALQQQLTPIFDPLFSDYSYGFRPGRSTHQAIEMARAHVTAGHRWCVELDLEKFFDRVNHDILMACI
ERRIKDKCVLRLIRRYLEAGIMSGGVVSPRQEGTPQGGPLSPLLSNILLDELDRELERRGHRFVRYADD
ANIYVRSPRAGERVLVSVERFLRERLKLTVNRKKSQVARAWKCDYLGYGMSWHQQPRLRVARMSLDRER
DRLRMLLRSVRARKMATVIERINPVLRGWASYFKLSQSKRPLEELDGWVRHKLRCVIWRQWKQPPTRER
NLMRLGLSEERANKSAFNGRGPWWNSGAQHMNYALPKKLWDRLGLVSILDTINRLSRNLNRRVRNRTHG
GVRGRRV
SEQ ID NO: 10
>CAB81565.1 putative reverse transcriptase-maturase-transposase
[Pseudomonas putida]
MTVIGSAAKTDAIGTGAPSHAERMWLQANWGLIKEDVKRLQARIAKATMEGRWGKVKALQHLLTRSHNG
KMLAVKRVTENRGKRTPGVDGKIWATPAAKSSGMESMRHRSYRALPLRRIYIPKSNGQKRPLGIPRMLC
RSMQALWKLALEPVSESLADPNSYGFRPNRSTADAIEYCFITLAKRTSPVWVLEGDIRGCFDNFNHEWM
LKNIPMDKTILRRWLQAGFIDEGTLFATQAGTPQGGIISPVIANMALDGLEAAVHASVGPTKRARERSK
INVVRYADDFVVTGISKEILEHSVLPAVRQFMAIRGLELSEEKTKITHIAEGFDFLGQNVRKYQGKLLI
KPANKSVKALLDKVREIVKSNKSATQANLILQLNPIIRGWAMYHRHVVSKSLFSSIDAQIWRLLWTWAL
RRHPNKGAGWVRQRYFHTVRYQNWVFRAQTKVGGIVQRWWLFRASTIPIVRHVKIRGLANPFDPAWSSY
FARRRSAMDVD
SEQ ID NO: 11
>CAC35989.1 putative reverse transcriptase and maturase
[Streptococcus agalactiae]
MQTTKKERNTHMSELLDKISSRNNMLEAYKQVKSNKGSAGIDGVTIEQMDDYLHQNWRETKKLIKERSY
KPQPVLRVEIPKPNGGVRNLGIPTAMDRMIQQAIVQVLSPLCEKHFSEYSYGFRPNRSCETAIVQLLEY
LNDGYEWIVDIDLEKFFDTVPQDRLMSLVHNIIQDGDTESLIRKYFHSGVVINGQRHKTLVGTPQGGNL
SPLLSNIMLNELDKGLEKRGLRFVRYADDCVITVGSEAAAKRVMHSVSSYIEKRLGLKVNMTKTKIVRP
NKLKYLGFGFWKSPKGWKCRPHQDSVQSFKRKLKQLTMRKWSIDLITRIERLNWVIRGWINYFSLGNMK
SIMTQIDERLRTRIRVIIWKQWKKKAKRLWGLLKLGVARWIADKVSGWGDHYQLVAQKSVLTRAISKPA
LAKRGLVSCLDYYLERHALKVS
SEQ ID NO: 12
>tr_D4L313_D4L313_9FIRM Retron-type reverse transcriptase
OS = Roseburia intestinalis XB6B4 OX = 718255 GN = RO1_37670 PE = 1 SV = 1
MVKSSGTERKERMDTSSLMEQILSNDNLNRAYLQVVRNKGAEGVDGMKYTELKEYLAKNG
EIIKEQLRIRKYKPQPVRRVEIPKPDGGVRNLGVPTVTDRFIQQAIAQVLTPIYEEQFHD
HSYGFRPNRCAQQAILTALDMMNDGNDWIVDIDLEKFFDTVNHDKLMTIIGRTIKDGDVI
SIVRKYLVSGIMIDDEYEDSIVGTPQGGNLSPLLANIMLNELDKEMEKRGLNFVRYADDC
IIMVGSEMSANRVMRNISRFIEEKLGLKVNMTKSKVDRPRGIKYLGFGFYYDTSAQQFKA
KPHAKSVMKYKKRMRELTCRSWGVSNSYKVERLNQLIRGWINYFKIGSMKTLCRELDGNI
RYRIRMCIWKHWKTPQNKEKNLVKLGVPRWAAHKVANTGNRYAHMCHNGWIQKAISTKRL
TSFGLVSMLDYYTERCVTC
SEQ ID NO: 13 (marathon)
>CBK92290.1 Retron-type reverse transcriptase [[Eubacterium] rectale
M104/1]
MDTSNLMEQILSSDNLNRAYLQVVRNKGAEGVDGMKYTELKEHLAKNGETIKGQLRTRKYKPQPARRVE
IPKPDGGVRNLGVPTVTDRFIQQAIAQVLTPIYEEQFHDHSYGFRPNRCAQQAILTALNIMNDGNDWIV
DIDLEKFFDTVNHDKLMTLIGRTIKDGDVISIVRKYLVSGIMIDDEYEDSIVGTPQGGNLSPLLANIML
NELDKEMEKRGLNFVRYADDCIIMVGSEMSANRVMRNISRFIEEKLGLKVNMTKSKVDRPSGLKYLGFG
FYFDPRAHQFKAKPHAKSVAKFKKRMKELTCRSWGVSNSYKVEKLNQLIRGWINYFKIGSMKTLCKELD
SRIRYRLRMCIWKQWKTPQNQEKNIVKLGIDRNTARRVAYTGKRIAYVQNKGAVNVAISNKRLASPGLI
SMLDYYIEKCVTC
SEQ ID NO: 14
>WP 013851921.1 group II intron reverse transcriptase/maturase
[Streptococcus pasteurianus]
MNSKMCATTNIANSWESIDFVKAEIYVKKLQMRIVKAWKLGKFNRVKSLQHLLTTSFYARALAVKRVTE
NQGKKTSGVDKELWLTPNAKYQAIKKLKVRGYCPKPLRRIYIPKKNGKKRPLSIPTMTDRAMQTLFKFA
LEPIAETTADPNSYGFRPKRSTQDAIEQCFLALSKQKSAKWVLEGDIKGCFDNISHEWIMKNIPMNKTI
LGKWLKSGYIENQKLFPTELGSPQGSPISPIISNMVLDGLERKLSATFRKKKVNGKVYTPKINFVRYAD
DFIVTGVSKELLENEVKPVIIEFLKERGLELSEEKTLITHITDGFDFLGINIRMYEGKLLTKPSKKNYE
SIASKIREVIKQNPSMKQELLIRKLNPSIIGWVNYQKHNVSTEAFQRLDNDIYQCLWRWCIRRHPKKGR
KWVANKYFHTFGSRSWIFSVQTTDTMENGEPFYLRLRCASDTDIRRHIKVKAEANPFDEQWQLYFEERQ
EKQMRQELKGRRVINGLYYKQKGVCPVCESKITKETDFRVHQTVKNHKPIKTLVHPTCHKNIKENTLVL
SEQ ID NO: 15
>WP_077124660.1 group II intron reverse transcriptase/maturase
[Shigella sonnei]
MNTHISVSTIPHLTGWHAINWKACHARVRKLQLRIAKATRQQQWRQVRELQRILTRSFSGKAVAVRRVT
ENTGKRTPGIDGKIWHTPKEKWGGVCSLNLRGYRPQPLRRIHIPKSNGKTRPLGIPTMRDRAMQALWLL
ALEPVSETTADHNSYGFRPMRSTHDAIESIFLRMSQKVSPKWILEGDIKGCFDNISHDWLLSHIPMDRR
LLKKWLKAGYMERGVFNHTNSGTPQGGIISPVLANMALDGLEKELMQTFRKSGYHSAKHQVNYVRYADD
FICSGSSRELLENEVRPLIAAFMRERGLELSEEKTAITHIDKGFDFLGQNVRKYNGKMLIKPSKKNLKN
FLCKVREIIKRNPTLPAWKLIGQLNPVIRGWATYHRHVVAKETFNYVDTQIWRAIWRWCVRRHPRKGLR
WIAGRYFSFEGRRWIFKAITPEGKILTLFRAMETPIKRHIKIKGEATPYTPGMEIYFERRLDLIWKGKS
KKMKTVVQLWKRQGKHCPQCGQPITNQTGWNIHHRIRKVMGGSDELTNLELLHPNCHRQLHSREAGAHR
KHL
----- group II intron yeast
SEQ ID NO: 16
>NP_009310.1 intron-encoded reverse transcriptase all (mitochondrion)
[Saccharomyces cerevisiae S288C]
MVQRWLYSTNAKDIAVLYFMLAIFSGMAGTAMSLIIRLELAAPGSQYLHGNSQLENGAPTSAYISLMRT
ALVLWIINRYLKHMTNSVGANFTGTMACHKTPMISVGGVKCYMVRLINFLQVFIRITISSYHLDMVKQV
WLFYVEVIRLWFIVIDSTGSVKKMKDTNNTKGNTKSEGSTERGNSGVDRGMVVPNTQMKMRFLNQVRYY
SVNNNLKMGKDTNIELSKDTSTSDLLEFEKLVMDNMNEENMNNNLLSIMKNVDMLMLAYNRIKSKPGNM
TPGTTLETLDGMNMMYLNKLSNELGTGKFKFKPMRMVNIPKPKGGMRPLSVGNPRDKIVQEVMRMILDT
IFDKKMSTHSHGFRKNMSCQTAIWEVRNMFGGSNWFIEVDLKKCFDTISHDLIIKELKRYISDKGFIDL
VYKLLRAGYIDEKGTYHKPMLGLPQGSLISPILCNIVMTLVDNWLEDYINLYNKGKVKKQHPTYKKLSR
MIAKAKMFSTRLKLHKERAKGPTFIYNDPNFKRMKYVRYADDILIGVLGSKNDCKMIKRDLNNFLNSLG
LTMNEEKTLITCATETPARFLGYNISITPLKRMPTVTKTIRGKTIRSRNTTRPIINAPIRDIINKLATN
GYCKHNKNGRMGVPTRVGRWTYEEPRTIINNYKALGRGILNYYKLATNYKRLRERIYYVLYYSCVLTLA
SKYRLKTMSKTIKKEGYNLNIIENDKLIANFPRNTFDNIKKIENHGMFMYMSEAKVTDPFEYIDSIKYM
LPTAKANFNKPCSICNSTIDVEMHHVKQLHRGMLKATKDYITGRMITMNRKQIPLCKQCHIKTHKNKFK
NMGPGM
SEQ ID NO: 17
>NP_009309.1 intron-encoded reverse transcriptase al2 (mitochondrion)
[Saccharomyces cerevisiae S288C]
MVQRWLYSTNAKDIAVLYFMLAIFSGMAGTAMSLIIRLELAAPGSQYLHGNSQLFNVLVVGHAVLMIFC
APFRLIYHCIEVLIDKHISVYSINENFTVSFWFWLLVVTYMVFRYVNHMAYPVGANSTGTMACHKSAGV
KQPAQGKNCPMARLINSCKECLGFSLTPSHLGIVIHAYVLEEEVHELTKNESLALSKSWHLEGCTSSNG
KLRNTGLSERGNPGDNGVFMVPKFNLNKVRYFSTLSKLNARKEDSLAYLTKINTTDFSELNKLMENNHN
KTETINTRILKLMSDIRMLLIAYNKIKSKKGNMSKGSNNITLDGINISYLNKLSKDINTNMFKFSPVRR
VEIPKTSGGFRPLSVGNPREKIVQESMRMMLEIIYNNSFSYYSHGFRPNLSCLTAIIQCKNYMQYCNWF
IKVDLNKCFDTIPHNMLINVLNERIKDKGFMDLLYKLLRAGYVDKNNNYHNTTIGIPQGSVVSPILCNI
FLDKLDKYLENKFENEFNTGNMSNRGRNPIYNSLSSKIYRCKLLSEKLKLIRLRDHYQRNMGSDKSFKR
AYFVRYADDIIIGVMGSHNDCKNILNDINNFLKENLGMSINMDKSVIKHSKEGVSFLGYDVKVTPWEKR
PYRMIKKGDNFIRVRHHTSLVVNAPIRSIVMKLNKHGYCSHGILGKPRGVGRLIHEEMKTILMHYLAVG
RGIMNYYRLATNFTTLRGRITYILFYSCCLTLARKFKLNTVKKVILKFGKVLVDPHSKVSFSIDDFKIR
HKMNMTDSNYTPDEILDRYKYMLPRSLSLFSGICQICGSKHDLEVHHVRTLNNAANKIKDDYLLGRMIK
MNRKQITICKTCHFKVHQGKYNGPGL
---- DGR (diversity generating retroelement)
SEQ ID NO: 18
>AAR97672. 1 reverse transcriptase [Bordetella virus BPP1]
MGKRHRNLIDQITTWENLLDAYRKTSHGKRRTWGYLEFKEYDLANLLALQAELKAGNYERGPYREFLVY
EPKPRLISALEFKDRIVQHALCNIVAPIFEAGLLPYTYACRPDKGTHAGVCHVQAELRRTRATHFLKSD
FSKFFPSIDRAALYAMIDKKIHCAATRRLLRVVLPDEGVGIPIGSLTSQLFANVYGGAVDRLLHDELKQ
RHWARYMDDIVVLGDDPEELRAVFYRLRDFASERLGLKISHWQVAPVSRGINFLGYRIWPTHKLLRKSS
VKRAKRKVANFIKHGEDESLQRFLASWSGHAQWADTHNLFTWMEEQYGIACH
SEQ ID NO: 19
>AJP62064. 1 reverse transcriptase [ANMV-1 virus]
MNAQQDNPTAKMETYKHLYTQICTKENICKAYRKARLGKRKKFYVRKFESDVDANIEQLHQQLRDESWT
PLPYKQFTAYEPKERLIRAPQFPDRIVHHALIRMLEPIYNKILIYDTYASRKNKGTHATVDRLTRFLRR
DNDNVFVFHGDVRKFFDNIDHETLIKILRKKIVDERVITLIKKILTNQGISLGVTLGNYTSQWFANIYL
SELDYFAKHNLKVKHYIRYMDDFLLLSDSKPELHRWKHQIEKFLNERLKLELHPVKRQIFPTNIGIDFV
GYTIWKDHKKLRRRDVNRFISRLNEFDKLPVMTPFAEASLMSWKGYSIHADAFGLTKQLHKSHPAMQVS
TLDRYIN
SEQ ID NO: 20
>DAC76693.1 TPA exp: reverse transcriptase [Bacteroides phage p00]
MRRVGYIIEEIVEPSNMEASFRQVLRGSKRKRSRQGCYLLAHKPEVLEELVAQIASGTFRVKDYREREI
IEGGKLRRIQVIPMKDRIAVHAIMAVVDRHLRKRFIRTTSASIKRRGMHDLLAYVRRDMAEDPDGTRYC
YKFDITKFYESVKQDFVMYCVSRVFKDAKLVTMLESFIRLMPEGLSIGLRSSQGLGNLLLSVYLDHYLK
DRYAVRHFYRYCDDGVVLGKTKAELWKIRDAVHGRMECAGLLVKGNERVFPPGEGIDFLGYVTFGADHV
RIRKRIKQKFARKMHEVKSRRRRRELIASFYGMAKHADCHTLFKKLTGKDMRSFKDLNVSYKPEDGKKR
FPGVVVSIRELVNLPIVVKDFETGIKTEQGEDRCIVAIEMNGEPKKFFINSEEMKNILLQVKDMPDGFP
FETTIKTETFGKGRTKYIFT
SEQ ID NO: 21
>AAS12785.1 reverse transcriptase family protein [Treponema denticola
ATCC 35405]
MKRKGNLYHKITEWNNLIAAFYNASRGKRLKPDVLLYEKNLYTNLKTLQNYLINQTVLLGSYRFFKIYD
PKERIICAAPFNERVLHHAIINITESVFEKFQIYDSYACRKNKGTQAALLRALYFSRRFKYFLKLDMKK
YFDSIPHSKLSLLLTCKFKDKALLHLENKLIASYSVTEGWGVPIGNLTSQYFANFYLSFFDHYAKEKMN
VRGYIRYMDDVLLFSDNLKDIKLIQKKAKNFLSCELDLTLKEEIIGMVKNGIPFLGFLVKPQGIYLSQK
KKKRLKKKIKDYVHKFKIAYWTEEEFALHITPVFAHIAISRCRAYCNKYLLT
SEQ ID NO: 22
>AJF63168.1 RNA-directed DNA polymerase Reverse transcriptase
[archaeon GW2011_AR20]
MQTYNKLFDKLCSYENLFLAYKKARKGKTGKGYVIKFEENLEDNLKILQFELINKIYKPKKLKLFIIRG
PKTRRICKSAFRDRIVHHAIINILEPVYEKIFIHDSYASRKNKGQHRALERFDYFKRIASKNGKKLKGI
RDKNYICGYCLKADIKKYFDNVNHETLINIIKKKIHDEDLIWLISQILGNKILGGDGKKGMPLGNYTSQ
FFANVYLNELDCFVKHNLKMRYYIRYVDDFVILYNDKETLEFYKREIDKFLKNKLKIELHEDKSKIIPL
HKGIHFLGFRNFYYYRLLKKSNINQIRRNLKEWNEAYKNDDGNLKTRTKGWKAHAKHGNNYKLAKILLN
A
---- Viral/Retroviral
SEQ ID NO: 23
>YP_009109694.1 reverse transcriptase [Baboon endogenous virus strain
M7]
TVSLQDEHRLFDIPVTTSLPDVWLQDFPQAWAETGGLGRAKCQAPIIIDLKPTAVPVSIKQYPMSLEAH
MGIRQHIIKFLELGVLRPCRSPWNTPLLPVKKPGTQDYRPVQDLREINKRTVDIHPTVPNPYNLLSTLK
PDYSWYTVLDLKDAFFCLPLAPQSQELFAFEWKDPERGISGQLTWTRLPQGFKNSPTLFDEALHRDLTD
FRTQHPEVTLLQYVDDLLLAAPTKKACTQGTRHLLQELGEKGYRASAKKAQICQTKVTYLGYILSEGKR
WLTPGRIETVARIPPPRNPREVREFLGTAGFCRLWIPGFAELAAPLYALTKESTPFTWQTEHQLAFEAL
KKALLSAPALGLPDTSKPFTLFLDERQGIAKGVLTQKLGPWKRPVAYLSKKLDPVAAGWPPCLRIMAAT
AMLVKDSAKLTLGQPLTVITPHTLEAIVRQPPDRWITNARLTHYQALLLDTDRVQFGPPVTLNPATLLP
VPENQPSPHDCRQVLAETHGTREDLKDQELPDADHTWYTDGSSYLDSGTRRAGAAVVDGHNTIWAQSLP
PGTSAQKAELIALTKALELSKGKKANIYTDSRYAFATAHTHGSIYERRGLUTSEGKEIKNKAEIIALLK
ALFLPQEVAIIHCPGHQKGQDPVAVGNRQADRVARQAAMAEVLTLATEPQNTSHIT
SEQ ID NO: 24
>NP_047255. 1:702-1370 Gag-Pro-Pol precursor polyprotein gPr80 [Feline
leukemia virus]
TLQLEEEYRLFEPESTQKQEMDIWLKNFPQAWAETGGMGTAHCQAPVLIQLKATATPISIRQYPMPHEA
YQGIKPHIRRMLDQGILKPCQSPWNTPLLPVKKPGTEDYRPVQDLREVNKRVEDIHPTVPNPYNLLSTL
PPSHPWYTVLDLKDAFFCLRLHSESQLLFAFEWRDPEIGLSGQLTWTRLPQGFKNSPTLFDEALHSDLA
DFRVRYPALVLLQYVDDLLLAAATRTECLEGTKALLETLGNKGYRASAKKAQICLQEVTYLGYSLKDGQ
RWLTKARKEAILSIPVPKNSRQVREFLGTAGYCRLWIPGFAELAAPLYPLTRPGTLFQWGTEQQLAFED
IKKALLSSPALGLPDITKPFELFIDENSGFAKGVIVQKLGPWKRPVAYLSKKLDTVASGWPPCLRMVAA
IAILVKDAGKLTLGQPLTILTSHPVEALVRQPPNKWLSNARMTHYQAMLLDAERVHFGPTVSLNPATEL
PLPSGGNHHDCLQILAETHGTRPDLTDQPLPDADLTWYTDGSSFIRNGEREAGAAVTTESEVIWAAPLP
PGTSAQRAELIALTQALKMAEGKKLTVYTDSRYAFATTHVHGEIYRRRGLLTSEGKEIKNKNEILALLE
ALFLPKRLSIIHCPGHQKGDSPQAKGNRLADDTAKKAATETHSSLTVL
SEQ ID NO: 25
>CAA68999.1 pol [Human foamy virus]
NQVGHRKIRPHNIATGDYPPRPQKQYPINPKAKPSIQIVIDDLLKQGVLTPQNSTMNTPVYPVPKPDGR
WRMVLDYREVNKTIPLTAAQNQHSAGILATIVRQKYKTTLDLANGFWAHPITPESYWLTAFTWQGKQYC
WTRLPQGFLNSPALFTADVVDLLKEIPNVQVYVDDIYLSHDDPKEHVQQLEKVFQILLQAGYVVSLKKS
EIGQKTVEFLGFNITKEGRGLTDTFKTKLINITPPKDLKQLQSILGLLNFARNFIPNFAELVQPLYNLI
ASAKGKYIEWSEENTKQLNMVIEALNTASNLEERLPEQRLVIKVNTSPSAGYVRYYNETGKKPIMYLNY
VFSKAELKFSMLEKLLTTMHKALIKAMDLAMGQEILVYSPIVSMTKIQKTPLPERKALPIRWITWMTYL
EDPRIQFHYDKTLPELKHIPDVYTSSQSPVKHPSQYEGVFYTDGSAIKSPDPTKSNNAGMGIVHATYKP
EYQVLNQWSIPLGNHTAQMAEIAAVEFACKKALKIPGPVLVITDSFYVAESANKELPYWKSNGFVNNKK
KPLKHISKWKSIAECLSMKPDITIQHEKGISLQIPVFILKGNALADKLATQGSYVVN
SEQ ID NO: 26
>AAB59937.1 pol polyprotein, partial [Feline immunodeficiency virus]
QISDKIPVVKVKMKDPNKGPQIKQWPLTNEKIEALTEIVERLEREGKVKRADPNNPWNTPVFAIKKKSG
KWRMLIDFRELNKLTEKGAEVQLGLPHPAGLQIKKQVTVLDIGDAYFTIPLDPDYAPYTAFTLPRKNNA
GPGRRFVWCSLPQGWILSPLIYQSTLDNIIQPFIRQNPQLDIYQYMDDIYIGSNLSKKEHKEKVEELRK
LLLWWGFETPEDKLQEEPPYTWMGYELHPLTWTIQQKQLDIPEQPTLNELQKLAGKINWASQAIPDLSI
KALTNMMRGNQNLNSTRQWTKEARLEVQKAKKAIEEQVQLGYYDPSKELYAKLSLVGPHQISYQVYQKD
PEKILWYGKMSRQKKKAENTCDIALRACYKIREESIIRIGKEPRYEIPTSREAWESNLINSPYLKAPPP
EVEYIHAALNIKRALSMIKDAPIPGAETWYIDGGRKLGKAAKAAYWTDTGKWQVMELEGSNQKAEIQAL
LLALKAGSEEMNIITDSQYVINIILQQPDMMEGIWQEVLEELEKKTAIFIDWVPGHKGIPGNEEVDKLC
QTMMIIEG
SEQ ID NO: 27
HERV-Kcon (Lee and Bieniasz, PLOS Pathog. 2007 Jan; 3(1): e10, sup.
FIG. 1)
KSRKRRNRVSFLGAATVEPPKPIPLTWKTEKPVWVNQWPLPKQKLEALHLLANEQLEKGHIEPSFSPWN
SPVFVIQKKSGKWRMLTDLRAVNAVIQPMGPLQPGLPSPAMIPKDWPLIIIDLKDCFFTIPLAEQDCEK
FAFTIPAINNKEPATRFQWKVLPQGMLNSPTICQTFVGRALQPVREKFSDCYIIHYIDDILCAAETKDK
LIDCYTFLQAEVANAGLAIASDKIQTSTPFHYLGMQIENRKIKPQKIEIRKDTLKTLNDFQKLLGDINW
IRPTLGIPTYAMSNLFSILRGDSDLNSKRMITPEATKEIKLVEEKIQSAQINRIDPLAPLQLLIFATAH
SPTGIIIQNTDLVEWSFLPHSTVKTFTLYLDQIATLIGQTRLRIIKLCGNDPDKIVVPLTKEQVRQAFI
NSGAWQIGLANFVGIIDNHYPKTKIFQFLKLTTWILPKITRREPLENALTVFTDGSSNGKAAYTGPKER
VIKTPYQSAQRAELVAVITVLQDFDQPINIISDSAYVVQATRDVETALIKYSMDDQLNQLENLLQQTVR
KRNFPFYITHIRAHTNLPGPLTKANEQADLLVSSALIKAQELHA
----- Eukaryotic group II introns
SEQ ID NO: 28
>XP_013295720. 1 reverse transcriptase [Necator americanus]
MDKAKPFSISKREVWEAYKQVKANRGAAGVDEQSMQEFEADLKNNLYRIWNRMSSGSYMPPPVLRVDIP
KAGGAGTRSLGIPTISDRIAQTVVKRYLESLVEPVFHDDSYGYRPGRSAHRALDVARQRCWSYAWALDL
DIKNFFGSIDWELMMRAVRRHTDCAWVLLYVERWLKARVQMPDGTVMQPDKGTPQGGVVSPVLANLFLH
YALDRWMQTHHPDVPFERYADDAIYHCKSEEQARLLRQEVEVRLAECKLAGHPEKTKIVYCKQANRPVD
YPTCQFDFLGYTFRPRSVMNRMGKLSVGFTPAVSNKAAKAMRQELRRKPLWHRSDLTLNDLADYTRPIL
RGWIQYYGRFSRSVLAQVLRYVDAALVRWARRKYKSLSRRPARAWTWLSGIRSRQPGLFAHWSVEAAVG
R
SEQ ID NO: 29
>CRX66588.1 putative reverse transcriptase protein (mitochondrion)
[Axinella verrucosa]
MRRLIWAGKGRRSTMDCYDVHMSTGLGRRESRLLNIASLFEAEGRQNACANRPRDIVPMAMAEWLKAIL
LLPSLDGGYLGRHGVSEMRRLLWICSRRVTRLAGDTISVHNEDNSRPKGTRPNPGNSGWPKGRNPYGHR
AGVVQGPASPGRPAVSASLTSRHYSTGSAPKVVRRLKGLTERCINHPNLAVDRNIYPLLCDPYLLTVAY
NNIRSKPGNMTPGVVPETLDGVSYETVKEISDGLRNETFQFKPGRKTQIPKQSGGLRSLTIAPPRDKIV
QEAMRILLNDIFEPTFSDLSHGFRPGRSCHTALQMIQQRFKPVTWMIEGDISKCFDSIDHGLLMAIIEK
KIKDRQFTKLIWKSLRAGYFEFHTIRHNIAGTPQGSIISPILSNIFMHQLDVFVEEMKAEFDRGSRARN
TAEYEHRRYLMKRAKRLGNTGELARIYKEAKKNPVMDFRDPSYKRLAYVRYADDWVVGVRGSYKEAERT
LDRITEFCRSISLTVSQSKTKITNLNKDKADFLGVNIFRSKHVKHSRKSSSAKQRQNLQLQFHVSIDRV
RSKLSSASIIRNGVAAPRFLWLPLSHRQIISLYNSVLRGYLNYYCFVGNHSRLVGWLRWTIYTSAAMLL
GRKYGLSTTKVFKRFGPRLSDGDTAGLHDPDYKATGKFRSKANPIVTGLYAKHVSIANLERLACEICGS
GYRVEMHHVRHMKDLNPAASVVDRLMARANRKQIPLCRECHMKRHRGEI
SEQ ID NO: 30
>CRX66589.1 putative reverse transcriptase protein (mitochondrion)
[Axinella verrucosa]
MCIIVLILGICIKAVSLPIRGQLGGDNSMLAKGGWKSSPRAKVAMVRLINPLTDEAGQKSRAAKRVVAS
NGIVCYIVVTIQRQSYARSASSILNIGEHLRSQYGMWWNSGNPESRKAGGFGGIVVLPSRGMATAGRKG
SRSKVSKEPGLAGFGKLEKLCEQIKVKESKGIGGLTEIMADPRFLGTSYQKRRSMPGMMTPGTDKVTLD
GISEKWFDEISQTFRNGLFKFRPVRRIGIPKPKGGVRYLGIPSPRDRIVQDAMKTLLELIFEPTESDAS
HGYRPGRGCHTALNHIKMKMGYVTWFIEGDISKCFDSVNHRRLMGIIEEAVSDQPFMDLIHKALKAGYI
EHPKGWVATNVGTPQGGVLSPLLANIYLDAFDKWMERKTESLEKGKRRRANPEYTKMIRESRVNREGYV
APLMGADENFKRVRYVRYADDFLIGVSGSLADCKNLRDEISEFLKRELELDLNLGKTRITHARSESAAP
LGYRIHITDPSKYAQRYVLRKGRYKWTHISTRPKMDAPIEKLVEKLGEQKFCKPGGRPTSNGKFIHESL
KEIIVRYRLLEKGLLNYYYMATNYGRVSARIHYTLKYSCALTIGRKMRLSTLKKVFKAYGKSLEVRDEK
GRCIASYPKISYARPAGKISTAVVSPFDLIGNCAKFWKRSLDSRGLQCAVCQATEGIEMHHVKHLRKSK
DMDWLTRRIVTMNRKQIPVCKECHQKIHRGRYDGRGLNRLIP
SEQ ID NO: 31
>RTX Reverse Transcriptase
MILDTDYITEDGKPVIRIFKKENGEFKIEYDRTFEPYLYALLKDDSAIEEVKKITAERHGTVVTVKRVE
KVQKKFLGRPVEVWKLYFTHPQDVPAIMDKIREHPAVIDIYEYDIPFAIRYLIDKGLVPMEGDEELKLL
AFDIETLYHEGEEFAEGPILMISYADEEGARVITWKNVDLPYVDVVSTEREMIKRFLRVVKEKDPDVLI
TYNGDNEDFAYLKKRCEKLGINFALGRDGSEPKIQRMGDRFAVEVKGRIHEDLYPVIRRTINLPTYTLE
AVYEAVFGQPKEKVYAEEITTAWETGENLERVARYSMEDAKVTYELGKEFLPMEAQLSRLIGQSLWDVS
RSSTGNLVEWFLLRKAYERNELAPNKPDEKELARRHQSHEGGYIKEPERGLWENIVYLDFRSLYPSIII
THNVSPDTUNREGCKEYDVAPQVGHRFCKDFPGFIPSLIGDLLEERQKIKKRMKATIDPIERKLLDYRQ
RAIKILANSLYGYYGYARARWYCKECAESVIAWGREYLTMTIKEIERKYGFKVIYSDTDGPPATIPGAD
AETVKKKAMEFLKYINAKLPGALELEYEGFYKRGLFVTKKKYAVIDEEGKITTRGLEIVRRDWSEIAKE
TQARVLEALLKDGDVEKAVRIVKEVTEKLSKYEVPPEKLVIHKQITRDLKDYKATGPHVAVAKRLAARG
VKIRPGTVISYIVLKGSGRIVDRAIPFDEFDPTKHKYDAEYYIEKQVLPAVERILRAFGYRKEDERYQK
TRQVGLSARLKPKGTLEGSSHHHHHH
SEQ ID NO: 32
PE2 (marathonRT)
MKRTADGSEFESPKKKRKVDKKYSIGLDIGTNSVGWAVITDEYKVPSKKFKVLGNTDRHSIKKNLIGAL
LEDSGETAEATRLKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFGNLIALSLGLTPNFKSNFDL
AEDAKLQLSKDTYDDDLDNLLAQIGDQYADLFLAAKNLSDAILLSDILRVNTEITKAPLSASMIKRYDE
HHQDLTLLKALVRQQLPEKYKEIFFDQSKNGYAGYIDGGASQEEFYKFIKPILEKMDGTEELLVKLNRE
DLLRKQRTFDNGSIPHQIHLGELHAILRRQEDFYPFLKDNREKIEKILTFRIPYYVGPLARGNSRPAWM
TRKSEETITPWNFEEVVDKGASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVEDRFNASLGTYHDLLKIIKD
KDFLDNEENEDILEDIVLTLTLFEDREMIEERLKTYAHLFDDKVMKQLKRRRYTGWGRLSRKLINGIRD
KQSGKTILDFLKSDGFANRNFMQLIHDDSLTFKEDIQKAQVSGQGDSLHEHIANLAGSPAIKKGILQTV
KVVDELVKVMGRHKPENIVIEMARENQTTQKGQKNSRERMKRIEEGIKELGSQILKEHPVENTQLQNEK
LYLYYLQNGRDMYVDQELDINRLSDYDVDAIVPQSFLKDDSIDNKVLTRSDKNRGKSDNVPSEEVVKKM
KNYWRQLLNAKLITQRKFDNLTKAERGGLSELDKAGFIKRQLVETRQITKHVAQILDSRMNTKYDENDK
LIREVKVITLKSKLVSDFRKDFQFYKVREINNYHHAHDAYLNAVVGTALIKKYPKLESEFVYGDYKVYD
VRKMIAKSEQEIGKATAKYFFYSNIMNFFKTEITLANGEIRKRPLIEINGETGEIVWDKGRDFATVRKV
LSMPQVNIVKKTEVQTGGFSKESILPKRNSDKLIARKKDWDPKKYGGFDSPTVAYSVLVVAKVEKGKSK
KLKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLSA
YNKHRDKPIREQAENIIHLFTLINIGAPAAFKYFDTTIDRKRYTSTKEVLDATLIHQSITGLYETRIDL
SQLGGDSGGSSGGSSGSETPGTSESATPESSGGSSGGSSDTSNIMEQILSSDNLNRAYLQVVRNKGAEG
VDGMKYTELKEHLAKNGETIKGQLRTRKYKPQPARRVEIPKPDGGVRNLGVPTVTDRFIQQAIAQVLTP
IYEEQFHDHSYGFRPNRCAQQAILTALNIMNDGNDWIVDIDLEKFFDTVNHDKLMTLIGRTIKDGDVIS
IVRKYLVSGIMIDDEYEDSIVGTPQGGNLSPLLANIMLNELDKEMEKRGLNFVRYADDCIIMVGSEMSA
NRVMRNISRFIEEKLGLKVNMTKSKVDRPSGLKYLGFGFYFDPRAHQFKAKPHAKSVAKFKKRMKELTC
RSWGVSNSYKVEKLNQLIRGWINYFKIGSMKTLCKELDSRIRYRLRMCIWKQWKTPQNQEKNLVKLGID
RNTARRVAYTGKRIAYVCNKGAVNVAISNKRLASFGLISMLDYYIEKCVTCSGGSKRTADGSEFEPKKK
RKV
SEQ ID NO: 33
PE2 (Human Foamy Virus)
MKRTADGSEFESPKKKRKVDKKYSIGLDIGTNSVGWAVITDEYKVPSKKFKVLGNTDRHSIKKNLIGAL
LEDSGETAEATRLKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDENPDNSDVDKLFIQLV
QTYNQLFEENPINASGVDAKAILSARLSKSRRLENLIAQLPGEKKNGLFGNLIALSLGLTPNFKSNFDL
AEDAKLQLSKDTYDDDLDNLLAQIGDQYADLFLAAKNLSDAILLSDILRVNTEITKAPLSASMIKRYDE
HHQDLTLLKALVRQQLPEKYKEIFFDQSKNGYAGYIDGGASQEEFYKFIKPILEKMDGTEELLVKLNRE
DLLRKQRTFDNGSIPHQIHLGELHAILRRQEDFYPFLKDNREKIEKILTFRIPYYVGPLARGNSRPAWM
TRKSEETITPWNFEEVVDKGASAQSFIERMINEDKNLPNEKVLPKHSLLYEYFTVYNELTKVKYVTEGM
RKPAFLSGEQKKAIVDLLFKTNRKVTVKQLKEDYFKKIECFDSVEISGVEDRFNASLGTYHDLLKIIKD
KDFLDNEENEDILEDIVLTLTLFEDREMIEERLKTYAHLFDDKVMKQLKRRRYTGWGRLSRKLINGIRD
KQSGKTILDFLKSDGFANRNFMQLIHDDSLTFKEDIQKAQVSGQGDSLHEHIANLAGSPAIKKGILQTV
KVVDELVKVMGRHKPENIVIEMARENQTTQKGQKNSRERMKRIEEGIKELGSQILKEHPVENTQLQNEK
LYLYYLQNGRDMYVDQELDINRLSDYDVDAIVPQSFLKDDSIDNKVLTRSDKNRGKSDNVPSEEVVKKM
KNYWRQLLNAKLITQRKFDNLTKAERGGLSELDKAGFIKRQLVETRQITKHVAQILDSRMNTKYDENDK
LIREVKVITLKSKLVSDFRKDFQFYKVREINNYHHAHDAYLNAVVGTALIKKYPKLESEFVYGDYKVYD
VRKMIAKSEQEIGKATAKYFFYSNIMNFFKTEITLANGEIRKRPLIETNGETGEIVWDKGRDFATVRKV
LSMPQVNIVKKTEVQTGGFSKESILPKRNSDKLIARKKDWDPKKYGGFDSPTVAYSVLVVAKVEKGKSK
KLKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLSA
YNKHRDKPIREQAENIIHLFTLTNLGAPAAFKYFDTTIDRKRYTSTKEVLDATLIHQSITGLYETRIDL
SQLGGDSGGSSGGSSGSETPGTSESATPESSGGSSGGSSNQVGHRKIRPHNIATGDYPPRPQKQYPINP
KAKPSIQIVIDDLLKQGVLTPQNSTMNTPVYPVPKPDGRWRMVLDYREVNKTIPLTAAQNQHSAGILAT
IVRQKYKTTLDLANGFWAHPITPESYWLTAFTWQGKQYCWTRLPQGFLNSPALFTADVVDLLKEIPNVQ
VYVDDIYLSHDDPKEHVQQLEKVFQILLQAGYVVSLKKSEIGQKTVEFLGFNITKEGRGLTDTFKTKLL
NITPPKDLKQLQSILGLLNFARNFIPNFAELVQPLYNLIASAKGKYIEWSEENTKQLNMVIEALNTASN
LEERLPEQRLVIKVNTSPSAGYVRYYNETGKKPIMYLNYVFSKAELKFSMLEKLLTTMHKALIKAMDLA
MGQEILVYSPIVSMTKIQKTPLPERKALPIRWITWMTYLEDPRIQFHYDKTLPELKHIPDVYTSSQSPV
KHPSQYEGVFYTDGSAIKSPDPTKSNNAGMGIVHATYKPEYQVLNQWSIPLGNHTAQMAEIAAVEFACK
KALKIPGPVLVITDSFYVAESANKELPYWKSNGFVNNKKKPLKHISKWKSIAECLSMKPDITIQHEKGI
SLQIPVFILKGNALADKLATQGSYVVNSGGSKRTADGSEFEPKKKRKV
SEQ ID NO: 34
PE2 (HERV-Kcon)
MKRTADGSEFESPKKKRKVDKKYSIGLDIGTNSVGWAVITDEYKVPSKKFKVLGNTDRHSIKKNLIGAL
LEDSGETAEATRLKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFGNLIALSLGLTPNFKSNFDL
AEDAKLQLSKDTYDDDLDNLLAQIGDQYADLFLAAKNLSDAILLSDILRVNTEITKAPLSASMIKRYDE
HHQDLTLLKALVRQQLPEKYKEIFFDQSKNGYAGYIDGGASQEEFYKFIKPILEKMDGTEELLVKLNRE
DLLRKQRTFDNGSIPHQIHLGELHAILRRQEDFYPFLKDNREKIEKILTFRIPYYVGPLARGNSRPAWM
TRKSEETITPWNFEEVVDKGASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVEDRFNASLGTYHDLLKIIKD
KDFLDNEENEDILEDIVLTLTLFEDREMIEERLKTYAHLFDDKVMKQLKRRRYTGWGRLSRKLINGIRD
KQSGKTILDFLKSDGFANRNFMQLIHDDSLTFKEDIQKAQVSGQGDSLHEHIANLAGSPAIKKGILQTV
KVVDELVKVMGRHKPENIVIEMARENQTTQKGQKNSRERMKRIEEGIKELGSQILKEHPVENTQLQNEK
LYLYYLQNGRDMYVDQELDINRLSDYDVDAIVPQSFLKDDSIDNKVLTRSDKNRGKSDNVPSEEVVKKM
KNYWRQLLNAKLITQRKFDNLTKAERGGLSELDKAGFIKRQLVETRQITKHVAQILDSRMNTKYDENDK
LIREVKVITLKSKLVSDFRKDFQFYKVREINNYHHAHDAYLNAVVGTALIKKYPKLESEFVYGDYKVYD
VRKMIAKSEQEIGKATAKYFFYSNIMNFFKTEITLANGEIRKRPLIETNGETGEIVWDKGRDFATVRKV
LSMPQVNIVKKTEVQTGGFSKESILPKRNSDKLIARKKDWDPKKYGGFDSPTVAYSVLVVAKVEKGKSK
KLKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLSA
YNKHRDKPIREQAENIIHLFTLTNLGAPAAFKYFDTTIDRKRYTSTKEVLDATLIHQSITGLYETRIDL
SQLGGDSGGSSGGSSGSETPGTSESATPESSGGSSGGSSKSRKRRNRVSFIGAATVEPPKPIPLTWKTE
KPVWVNQWPLPKQKLEALHLLANEQLEKGHIEPSFSPWNSPVFVIQKKSGKWRMLTDLRAVNAVIQPMG
PLQPGLPSPAMIPKDWPLIIIDLKDCFFTIPLAEQDCEKFAFTIPAINNKEPATRFQWKVLPQGMLNSP
TICQTFVGRALQPVREKFSDCYIIHYIDDILCAAETKDKLIDCYTFLQAEVANAGLAIASDKIQTSTPF
HYLGMQIENRKIKPQKIEIRKDTLKTLNDFQKLLGDINWIRPTLGIPTYAMSNLFSILRGDSDLNSKRM
LTPEATKEIKLVEEKIQSAQINRIDPLAPLQLLIFATAHSPTGIIIQNTDLVEWSFLPHSTVKTFTLYL
DQIATLIGQTRLRIIKLCGNDPDKIVVPLTKEQVRQAFINSGAWQIGLANFVGIIDNHYPKTKIFQFLK
LTTWILPKITRREPLENALTVFTDGSSNGKAAYTGPKERVIKTPYQSAQRAELVAVITVLQDFDQPINI
ISDSAYVVQATRDVETALIKYSMDDQLNQLFNLLQQTVRKRNFPFYITHIRAHTNLPGPLTKANEQADL
LVSSALIKAQELHASGGSKRTADGSEFEPKKKRKV

TABLE D
Improved CRISPR Prime Editors Sequence Table

| | | SEQ | | SEQ |
|---|---|---|---|---|
| | | ID | | ID |
| Construct | Nucleotide sequence | NO: | Amino acid sequence | NO: |
| bpNLS-MMLV | ATGAAACGGACAGCCGACGGAAGCGAGTTCG | 48 | MKRTADGSEFESPKKKRKVTLNIEDEYRLH | 49 |
| RT-4 AA | AGTCACCAAAGAAGAAGCGGAAAGTCACCCT | | ETSKEPDVSLGSTWLSDFPQAWAETGGMGL | |
| linker- | AAATATAGAAGATGAGTATCGGCTACATGAG | | AVRQAPLIIPLKATSTPVSIKQYPMSQEAR | |
| bpNLS | ACCTCAAAAGAGCCAGATGTTTCTCTAGGGT | | LGIKPHIQRLLDQGILVPCQSPWNTPLLPV | |
| | CCACATGGCTGTCTGATTTTCCTCAGGCCTG | | KKPGTNDYRPVQDLREVNKRVEDIHPTVPN | |
| | GGCGGAAACCGGGGGCATGGGACTGGCAGTT | | PYNLLSGLPPSHQWYTVLDLKDAFFCERLH | |
| | CGCCAAGCTCCTCTGATCATACCTCTGAAAG | | PTSQPLFAFEWRDPEMGISGQLTWTRLPQG | |
| | CAACCTCTACCCCCGTGTCCATAAAACAATA | | FKNSPTLFNEALHRDLADFRIQHPDLILLQ | |
| | CCCCATGTCACAAGAAGCCAGACTGGGGATC | | YVDDLLLAATSELDCQQGTRALLQTLGNLG | |
| | AAGCCCCACATACAGAGACTGTTGGACCAGG | | YRASAKKAQICQKQVKYLGYLLKEGQRWLT | |
| | GAATACTGGTACCCTGCCAGTCCCCCTGGAA | | EARKETVMGQPTPKTPRQLREFLGKAGFCR | |
| | CACGCCCCTGCTACCCGTTAAGAAACCAGGG | | LFIPGFAEMAAPLYPLTKPGTLENWGPDQQ | |
| | ACTAATGATTATAGGCCTGTCCAGGATCTGA | | KAYQEIKQALLTAPALGLPDLTKPFELFVD | |
| | GAGAAGTCAACAAGCGGGTGGAAGACATCCA | | EKQGYAKGVLTQKLGPWRRPVAYLSKKLDP | |
| | CCCCACCGTGCCCAACCCTTACAACCTCTTG | | VAAGWPPCLRMVAAIAVLTKDAGKLIMGQP | |
| | AGCGGGCTCCCACCGTCCCACCAGTGGTACA | | LVILAPHAVEALVKQPPDRWLSNARMTHYQ | |
| | CTGTGCTTGATTTAAAGGATGCCTTTTTCTG | | ALLLDTDRVQFGPVVALNPATLLPLPEEGL | |
| | CCTGAGACTCCACCCCACCAGTCAGCCTCTC | | QHNCLDILAEAHGTRPDLTDQPLPDADHTW | |
| | TTCGCCTTTGAGTGGAGAGATCCAGAGATGG | | YTDGSSLLQEGQRKAGAAVTTETEVIWAKA | |
| | GAATCTCAGGACAATTGACCTGGACCAGACT | | LPAGTSACRAELIALTQALKMAEGKKENVY | |
| | CCCACAGGGTTTCAAAAACAGTCCCACCCTG | | TDSRYAFATAHIHGEIYRRRGWLTSEGKEI | |
| | TTTAATGAGGCACTGCACAGAGACCTAGCAG | | KNKDEILALLKALFLPKRLSIIHCPGHQKG | |
| | ACTTCCGGATCCAGCACCCAGACTTGATCCT | | HSAEARGNRMADQAARKAAITETPDTSTLL | |
| | GCTACAGTACGTGGATGACTTACTGCTGGCC | | IENSSPSGGSKRTADGSEFEPKKKRKV* | |
| | GCCACTTCTGAGCTAGACTGCCAACAAGGTA | | | |
| | CTCGGGCCCTGTTACAAACCCTAGGGAACCT | | | |
| | CGGGTATCGGGCCTCGGCCAAGAAAGCCCAA | | | |
| | ATTTGCCAGAAACAGGTCAAGTATCTGGGGT | | | |
| | ATCTTCTAAAAGAGGGTCAGAGATGGCTGAC | | | |
| | TGAGGCCAGAAAAGAGACTGTGATGGGGCAG | | | |
| | CCTACTCCGAAGACCCCTCGACAACTAAGGG | | | |
| | AGTTCCTAGGGAAGGCAGGCTTCTGTCGCCT | | | |
| | CTTCATCCCTGGGTTTGCAGAAATGGCAGCC | | | |
| | CCCCTGTACCCTCTCACCAAACCGGGGACTC | | | |
| | TGTTTAATTGGGGCCCAGACCAACAAAAGGC | | | |
| | CTATCAAGAAATCAAGCAAGCTCTTCTAACT | | | |
| | GCCCCAGCCCTGGGGTTGCCAGATTTGACTA | | | |
| | AGCCCTTTGAACTCTTTGTCGACGAGAAGCA | | | |
| | GGGCTACGCCAAAGGTGTCCTAACGCAAAAA | | | |
| | CTGGGACCTTGGCGTCGGCCGGTGGCCTACC | | | |
| | TGTCCAAAAAGCTAGACCCAGTAGCAGCTGG | | | |
| | GTGGCCCCCTTGCCTACGGATGGTAGCAGCC | | | |
| | ATTGCCGTACTGACAAAGGATGCAGGCAAGC | | | |
| | TAACCATGGGACAGCCACTAGTCATTCTGGC | | | |
| | CCCCCATGCAGTAGAGGCACTAGTCAAACAA | | | |
| | CCCCCCGACCGCTGGCTTTCCAACGCCCGGA | | | |
| | TGACTCACTATCAGGCCTTGCTTTTGGACAC | | | |
| | GGACCGGGTCCAGTTCGGACCGGTGGTAGCC | | | |
| | CTGAACCCGGCTACGCTGCTCCCACTGCCTG | | | |
| | AGGAAGGGCTGCAACACAACTGCCTTGATAT | | | |
| | CCTGGCCGAAGCCCACGGAACCCGACCCGAC | | | |
| | CTAACGGACCAGCCGCTCCCAGACGCCGACC | | | |
| | ACACCTGGTACACGGATGGAAGCAGTCTCTT | | | |
| | ACAAGAGGGACAGCGTAAGGGGGGAGCTGCG | | | |
| | GTGACCACCGAGACCGAGGTAATCTGGGCTA | | | |
| | AAGCCCTGCCAGCCGGGACATCCGCTCAGCG | | | |
| | GGCTGAACTGATAGCACTCACCCAGGCCCTA | | | |
| | AAGATGGCAGAAGGTAAGAAGCTAAATGTTT | | | |
| | ATACTGATAGCCGTTATGCTTTTGCTACTGC | | | |
| | CCATATCCATGGAGAAATATACAGAAGGCGT | | | |
| | GGGTGGCTCACATCAGAAGGCAAAGAGATCA | | | |
| | AAAATAAAGACGAGATCTTGGCCCTACTAAA | | | |
| | AGCCCTCTTTCTGCCCAAAAGACTTAGCATA | | | |
| | ATCCATTGTCCAGGACATCAAAAGGGACACA | | | |
| | GCGCCGAGGCTAGAGGCAACCGGATGGCTGA | | | |
| | CCAAGCGGCCCGAAAGGCAGCCATCACAGAG | | | |
| | ACTCCAGACACCTCTACCCTCCTCATAGAAA | | | |
| | ATTCATCACCCTCTGGCGGCTCAAAAAGAAC | | | |
| | CGCCGACGGCAGCGAATTCGAGCCCAAGAAG | | | |
| | AAGAGGAAAGTCTAA | | | |
| bpNLS-MMLV | ATGAAACGGACAGCCGACGGAAGCGAGTTCG | 50 | MKRTADGSEFESPKKKRKVTLNIEDEYRER | 51 |
| RT | AGTCACCAAAGAAGAAGCGGAAAGTCACCCT | | ETSKEPDVSLGSTWLSDFPQAWAETGGMGL | |
| (246 AA | AAATATAGAAGATGAGTATCGGCTACATGAG | | AVRQAPLIIPLKATSTPVSIKQYPMSQEAR | |
| truncation)- | ACCTCAAAAGAGCCAGATGTTTCTCTAGGGT | | LGIKPHIQRLLDQGILVPCQSPWNTPLLPV | |
| 4 AA | CCACATGGCTGTCTGATTTTCCTCAGGCCTG | | KKPGTNDYRPVQDLREVNKRVEDIHPTVPN | |
| linker- | GGCGGAAACCGGGGGCATGGGACTGGCAGTT | | PYNLLSGLPPSHQWYTVLDLKDAFFCERLH | |
| bpNLS | CGCCAAGCTCCTCTGATCATACCTCTGAAAG | | PTSQPLFAFEWRDPEMGISGQLTWTRLPQG | |
| | CAACCTCTACCCCCGTGTCCATAAAACAATA | | FKNSPTLFNEALHRDLADFRIQHPDLILLQ | |
| | CCCCATGTCACAAGAAGCCAGACTGGGGATC | | YVDDLLLAATSELDCQQGTRALLQTLGNLG | |
| | AAGCCCCACATACAGAGACTGTTGGACCAGG | | YRASAKKAQICQKQVKYLGYLLKEGQRWLT | |
| | GAATACTGGTACCCTGCCAGTCCCCCTGGAA | | EARKETVMGQPTPKTPRQLREFLGKAGFCR | |
| | CACGCCCCTGCTACCCGTTAAGAAACCAGGG | | LFIPGFAEMAAPLYPLTKPGTLENWGPDQQ | |
| | ACTAATGATTATAGGCCTGTCCAGGATCTGA | | KAYQEIKQALLTAPALGLPDLTKPFELFVD | |
| | GAGAAGTCAACAAGCGGGTGGAAGACATCCA | | EKQGYAKGVLTQKLGPWRRPVAYLSKKLDP | |
| | CCCCACCGTGCCCAACCCTTACAACCTCTTG | | VAAGWPPCLRMVAAIAVLTKDAGKLTMGQP | |
| | AGCGGGCTCCCACCGTCCCACCAGTGGTACA | | SGGSKRTADGSEFEPKKKRKV* | |
| | CTGTGCTTGATTTAAAGGATGCCTTTTTCTG | | | |
| | CCTGAGACTCCACCCCACCAGTCAGCCTCTC | | | |
| | TTCGCCTTTGAGTGGAGAGATCCAGAGATGG | | | |
| | GAATCTCAGGACAATTGACCTGGACCAGACT | | | |
| | CCCACAGGGTTTCAAAAACAGTCCCACCCTG | | | |
| | TTTAATGAGGCACTGCACAGAGACCTAGCAG | | | |
| | ACTTCCGGATCCAGCACCCAGACTTGATCCT | | | |
| | GCTACAGTACGTGGATGACTTACTGCTGGCC | | | |
| | GCCACTTCTGAGCTAGACTGCCAACAAGGTA | | | |
| | CTCGGGCCCTGTTACAAACCCTAGGGAACCT | | | |
| | CGGGTATCGGGCCTCGGCCAAGAAAGCCCAA | | | |
| | ATTTGCCAGAAACAGGTCAAGTATCTGGGGT | | | |
| | ATCTTCTAAAAGAGGGTCAGAGATGGCTGAC | | | |
| | TGAGGCCAGAAAAGAGACTGTGATGGGGCAG | | | |
| | CCTACTCCGAAGACCCCTCGACAACTAAGGG | | | |
| | AGTTCCTAGGGAAGGCAGGCTTCTGTCGCCT | | | |
| | CTTCATCCCTGGGTTTGCAGAAATGGCAGCC | | | |
| | CCCCTGTACCCTCTCACCAAACCGGGGACTC | | | |
| | TGTTTAATTGGGGCCCAGACCAACAAAAGGC | | | |
| | CTATCAAGAAATCAAGCAAGCTCTTCTAACT | | | |
| | GCCCCAGCCCTGGGGTTGCCAGATTTGACTA | | | |
| | AGCCCTTTGAACTCTTTGTCGACGAGAAGCA | | | |
| | GGGCTACGCCAAAGGTGTCCTAACGCAAAAA | | | |
| | CTGGGACCTTGGCGTCGGCCGGTGGCCTACC | | | |
| | TGTCCAAAAAGCTAGACCCAGTAGCAGCTGG | | | |
| | GTGGCCCCCTTGCCTACGGATGGTAGCAGCC | | | |
| | ATTGCCGTACTGACAAAGGATGCAGGCAAGC | | | |
| | TAACCATGGGACAGCCATCTGGCGGCTCAAA | | | |
| | AAGAACCGCCGACGGCAGCGAATTCGAGCCC | | | |
| | AAGAAGAAGAGGAAAGTCTAA | | | |
| bpNLS-MMLV | ATGAAACGGACAGCCGACGGAAGCGAGTTCG | 52 | MKRTADGSEFESPKKKRKVTWLSDFPQAWA | 53 |
| RT | AGTCACCAAAGAAGAAGCGGAAAGTCACATG | | ETGGMGLAVRQAPLIIPLKATSTPVSIKQY | |
| (23 AA | GCTGTCTGATTTTCCTCAGGCCTGGGCGGAA | | PMSQEARLGIKPHIQRLLDQGILVPCQSPW | |
| truncation)- | ACCGGGGGCATGGGACTGGCAGTTCGCCAAG | | NTPLLPVKKPGTNDYRPVQDLREVNKRVED | |
| 4 AA | CTCCTCTGATCATACCTCTGAAAGCAACCTC | | IHPTVPNPYNLLSGLPPSHQWYTVLDLKDA | |
| linker-bpNLS | TACCCCCGTGTCCATAAAACAATACCCCATG | | FFCLRLHPTSQPLFAFEWRDPEMGISGQLT | |
| | TCACAAGAAGCCAGACTGGGGATCAAGCCCC | | WTRLPQGFKNSPTLFNEALHRDLADFRIQH | |
| | ACATACAGAGACTGTTGGACCAGGGAATACT | | PDLILLQYVDDLLLAATSELDCQQGTRALL | |
| | GGTACCCTGCCAGTCCCCCTGGAACACGCCC | | QTLGNLGYRASAKKAQICQKQVKYLGYLLK | |
| | CTGCTACCCGTTAAGAAACCAGGGACTAATG | | EGQRWLTEARKETVMGQPTPKTPRQLREFL | |
| | ATTATAGGCCTGTCCAGGATCTGAGAGAAGT | | GKAGFCRLFIPGFAEMAAPLYPLTKPGTLF | |
| | CAACAAGCGGGTGGAAGACATCCACCCCACC | | NWGPDQQKAYQEIKQALLTAPALGLPDLTK | |
| | GTGCCCAACCCTTACAACCTCTTGAGCGGGC | | PFELFVDEKQGYAKGVLIQKLGPWRRPVAY | |
| | TCCCACCGTCCCACCAGTGGTACACTGTGCT | | LSKKLDPVAAGWPPCLRMVAAIAVLTKDAG | |
| | TGATTTAAAGGATGCCTTTTTCTGCCTGAGA | | KLTMGQPLVILAPHAVEALVKQPPDRWLSN | |
| | CTCCACCCCACCAGTCAGCCTCTCTTCGCCT | | ARMTHYQALLLDTDRVQFGPVVALNPATLL | |
| | TTGAGTGGAGAGATCCAGAGATGGGAATCTC | | PLPEEGLQHNCLDILAEAHGTRPDLTDQPL | |
| | AGGACAATTGACCTGGACCAGACTCCCACAG | | PDADHTWYTDGSSLLQEGQRKAGAAVTTET | |
| | GGTTTCAAAAACAGTCCCACCCTGTTTAATG | | EVIWAKALPAGTSAQRAELIALTQALKMAE | |
| | AGGCACTGCACAGAGACCTAGCAGACTTCCG | | GKKLNVYTDSRYAFATAHIHGEIYRRRGWL | |
| | GATCCAGCACCCAGACTTGATCCTGCTACAG | | TSEGKEIKNKDEILALLKALFLPKRLSIIH | |
| | TACGTGGATGACTTACTGCTGGCCGCCACTT | | CPGHQKGHSAEARGNRMADQAARKAAITET | |
| | CTGAGCTAGACTGCCAACAAGGTACTCGGGC | | PDTSTLLIENSSPSGGSKRTADGSEFEPKK | |
| | CCTGTTACAAACCCTAGGGAACCTCGGGTAT | | KRKV* | |
| | CGGGCCTCGGCCAAGAAAGCCCAAATTTGCC | | | |
| | AGAAACAGGTCAAGTATCTGGGGTATCTTCT | | | |
| | AAAAGAGGGTCAGAGATGGCTGACTGAGGCC | | | |
| | AGAAAAGAGACTGTGATGGGGCAGCCTACTC | | | |
| | CGAAGACCCCTCGACAACTAAGGGAGTTCCT | | | |
| | AGGGAAGGCAGGCTTCTGTCGCCTCTTCATC | | | |
| | CCTGGGTTTGCAGAAATGGCAGCCCCCCTGT | | | |
| | ACCCTCTCACCAAACCGGGGACTCTGTTTAA | | | |
| | TTGGGGCCCAGACCAACAAAAGGCCTATCAA | | | |
| | GAAATCAAGCAAGCTCTTCTAACTGCCCCAG | | | |
| | CCCTGGGGTTGCCAGATTTGACTAAGCCCTT | | | |
| | TGAACTCTTTGTCGACGAGAAGCAGGGCTAC | | | |
| | GCCAAAGGTGTCCTAACGCAAAAACTGGGAC | | | |
| | CTTGGCGTCGGCCGGTGGCCTACCTGTCCAA | | | |
| | AAAGCTAGACCCAGTAGCAGCTGGGTGGCCC | | | |
| | CCTTGCCTACGGATGGTAGCAGCCATTGCCG | | | |
| | TACTGACAAAGGATGCAGGCAAGCTAACCAT | | | |
| | GGGACAGCCACTAGTCATTCTGGCCCCCCAT | | | |
| | GCAGTAGAGGCACTAGTCAAACAACCCCCCG | | | |
| | ACCGCTGGCTTTCCAACGCCCGGATGACTCA | | | |
| | CTATCAGGCCTTGCTTTTGGACACGGACCGG | | | |
| | GTCCAGTTCGGACCGGTGGTAGCCCTGAACC | | | |
| | CGGCTACGCTGCTCCCACTGCCTGAGGAAGG | | | |
| | GCTGCAACACAACTGCCTTGATATCCTGGCC | | | |
| | GAAGCCCACGGAACCCGACCCGACCTAACGG | | | |
| | ACCAGCCGCTCCCAGACGCCGACCACACCTG | | | |
| | GTACACGGATGGAAGCAGTCTCTTACAAGAG | | | |
| | GGACAGCGTAAGGCGGGAGCTGCGGTGACCA | | | |
| | CCGAGACCGAGGTAATCTGGGCTAAAGCCCT | | | |
| | GCCAGCCGGGACATCCGCTCAGCGGGCTGAA | | | |
| | CTGATAGCACTCACCCAGGCCCTAAAGATGG | | | |
| | CAGAAGGTAAGAAGCTAAATGTTTATACTGA | | | |
| | TAGCCGTTATGCTTTTGCTACTGCCCATATC | | | |
| | CATGGAGAAATATACAGAAGGCGTGGGTGGC | | | |
| | TCACATCAGAAGGCAAAGAGATCAAAAATAA | | | |
| | AGACGAGATCTTGGCCCTACTAAAAGCCCTC | | | |
| | TTTCTGCCCAAAAGACTTAGCATAATCCATT | | | |
| | GTCCAGGACATCAAAAGGGACACAGCGCCGA | | | |
| | GGCTAGAGGCAACCGGATGGCTGACCAAGCG | | | |
| | GCCCGAAAGGCAGCCATCACAGAGACTCCAG | | | |
| | ACACCTCTACCCTCCTCATAGAAAATTCATC | | | |
| | ACCCTCTGGCGGCTCAAAAAGAACCGCCGAC | | | |
| | GGCAGCGAATTCGAGCCCAAGAAGAAGAGGA | | | |
| | AAGTCTAA | | | |
| bpNLS-MMLV | ATGAAACGGACAGCCGACGGAAGCGAGTTCG | 54 | MKRTADGSEFESPKKKRKVTLNIEDEYRLH | 55 |
| RT | AGTCACCAAAGAAGAAGCGGAAAGTCACCCT | | ETSKEPDVSLGSTWLSDFPQAWAETGGMGL | |
| (207 AA | AAATATAGAAGATGAGTATCGGCTACATGAG | | AVRQAPLIIPLKATSTPVSIKQYPMSQEAR | |
| truncation)- | ACCTCAAAAGAGCCAGATGTTTCTCTAGGGT | | LGIKPHIQRLLDQGILVPCQSPWNTPLLPV | |
| 4 AA | CCACATGGCTGTCTGATTTTCCTCAGGCCTG | | KKPGTNDYRPVQDLREVNKRVEDIHPTVPN | |
| linker-bpNLS | GGCGGAAACCGGGGGCATGGGACTGGCAGTT | | PYNLLSGLPPSHQWYTVLDLKDAFFCERLH | |
| | CGCCAAGCTCCTCTGATCATACCTCTGAAAG | | PTSQPLFAFEWRDPEMGISGQLTWIRLPQG | |
| | CAACCTCTACCCCCGTGTCCATAAAACAATA | | FKNSPTLFNEALHRDLADFRIQHPDLILLQ | |
| | CCCCATGTCACAAGAAGCCAGACTGGGGATC | | YVDDLLLAATSELDCQQGTRALLQTLGNLG | |
| | AAGCCCCACATACAGAGACTGTTGGACCAGG | | YRASAKKAQICQKQVKYLGYLLKEGQRWLT | |
| | GAATACTGGTACCCTGCCAGTCCCCCTGGAA | | EARKETVMGQPTPKTPRQLREFLGKAGFCR | |
| | CACGCCCCTGCTACCCGTTAAGAAACCAGGG | | LFIPGFAEMAAPLYPLTKPGTLENWGPDQQ | |
| | ACTAATGATTATAGGCCTGTCCAGGATCTGA | | KAYQEIKQALLTAPALGLPDLTKPFELFVD | |
| | GAGAAGTCAACAAGCGGGTGGAAGACATCCA | | EKQGYAKGVLTQKLGPWRRPVAYLSKKLDP | |
| | CCCCACCGTGCCCAACCCTTACAACCTCTTG | | VAAGWPPCLRMVAAIAVLTKDAGKLTMGQP | |
| | AGCGGGCTCCCACCGTCCCACCAGTGGTACA | | LVILAPHAVEALVKQPPDRWLSNARMTHYQ | |
| | CTGTGCTTGATTTAAAGGATGCCTTTTTCTG | | ALLLDIDRVSGGSKRTADGSEFEPKKKRKV | |
| | CCTGAGACTCCACCCCACCAGTCAGCCTCTC | | * | |
| | TTCGCCTTTGAGTGGAGAGATCCAGAGATGG | | | |
| | GAATCTCAGGACAATTGACCTGGACCAGACT | | | |
| | CCCACAGGGTTTCAAAAACAGTCCCACCCTG | | | |
| | TTTAATGAGGCACTGCACAGAGACCTAGCAG | | | |
| | ACTTCCGGATCCAGCACCCAGACTTGATCCT | | | |
| | GCTACAGTACGTGGATGACTTACTGCTGGCC | | | |
| | GCCACTTCTGAGCTAGACTGCCAACAAGGTA | | | |
| | CTCGGGCCCTGTTACAAACCCTAGGGAACCT | | | |
| | CGGGTATCGGGCCTCGGCCAAGAAAGCCCAA | | | |
| | ATTTGCCAGAAACAGGTCAAGTATCTGGGGT | | | |
| | ATCTTCTAAAAGAGGGTCAGAGATGGCTGAC | | | |
| | TGAGGCCAGAAAAGAGACTGTGATGGGGCAG | | | |
| | CCTACTCCGAAGACCCCTCGACAACTAAGGG | | | |
| | AGTTCCTAGGGAAGGCAGGCTTCTGTCGCCT | | | |
| | CTTCATCCCTGGGTTTGCAGAAATGGCAGCC | | | |
| | CCCCTGTACCCTCTCACCAAACCGGGGACTC | | | |
| | TGTTTAATTGGGGCCCAGACCAACAAAAGGC | | | |
| | CTATCAAGAAATCAAGCAAGCTCTTCTAACT | | | |
| | GCCCCAGCCCTGGGGTTGCCAGATTTGACTA | | | |
| | AGCCCTTTGAACTCTTTGTCGACGAGAAGCA | | | |
| | GGGCTACGCCAAAGGTGTCCTAACGCAAAAA | | | |
| | CTGGGACCTTGGCGTCGGCCGGTGGCCTACC | | | |
| | TGTCCAAAAAGCTAGACCCAGTAGCAGCTGG | | | |
| | GTGGCCCCCTTGCCTACGGATGGTAGCAGCC | | | |
| | ATTGCCGTACTGACAAAGGATGCAGGCAAGC | | | |
| | TAACCATGGGACAGCCACTAGTCATTCTGGC | | | |
| | CCCCCATGCAGTAGAGGCACTAGTCAAACAA | | | |
| | CCCCCCGACCGCTGGCTTTCCAACGCCCGGA | | | |
| | TGACTCACTATCAGGCCTTGCTTTTGGACAC | | | |
| | GGACCGGGTCTCTGGCGGCTCAAAAAGAACC | | | |
| | GCCGACGGCAGCGAATTCGAGCCCAAGAAGA | | | |
| | AGAGGAAAGTCTAA | | | |
| bpNLS-MMLV | ATGAAACGGACAGCCGACGGAAGCGAGTTCG | 56 | MKRTADGSEFESPKKKRKVTLNIEDEYRLH | 57 |
| RT | AGTCACCAAAGAAGAAGCGGAAAGTCACCCT | | ETSKEPDVSLGSTWLSDFPQAWAETGGMGL | |
| (316 AA | AAATATAGAAGATGAGTATCGGCTACATGAG | | AVRQAPLIIPLKATSTPVSIKQYPMSQEAR | |
| truncation)- | ACCTCAAAAGAGCCAGATGTTTCTCTAGGGT | | LGIKPHIQRLLDQGILVPCQSPWNTPLLPV | |
| 4 AA | CCACATGGCTGTCTGATTTTCCTCAGGCCTG | | KKPGTNDYRPVQDLREVNKRVEDIHPTVPN | |
| linker- | GGCGGAAACCGGGGGCATGGGACTGGCAGTT | | PYNLLSGLPPSHQWYTVLDLKDAFFCERER | |
| bpNLS | CGCCAAGCTCCTCTGATCATACCTCTGAAAG | | PTSQPLFAFEWRDPEMGISGQLTWTRLPQG | |
| | CAACCTCTACCCCCGTGTCCATAAAACAATA | | FKNSPTLFNEALHRDLADFRIQHPDLILLQ | |
| | CCCCATGTCACAAGAAGCCAGACTGGGGATC | | YVDDLLLAATSELDCQQGTRALLQTLGNLG | |
| | AAGCCCCACATACAGAGACTGTTGGACCAGG | | YRASAKKAQICQKQVKYLGYLLKEGQRWLT | |
| | GAATACTGGTACCCTGCCAGTCCCCCTGGAA | | EARKETVMGQPTPKTPRQLREFLGKAGFCR | |
| | CACGCCCCTGCTACCCGTTAAGAAACCAGGG | | LFIPGFAEMAAPLYPLTKPGTLENWGPDQQ | |
| | ACTAATGATTATAGGCCTGTCCAGGATCTGA | | KAYQEIKQALLTAPALGLPDSGGSKRTADG | |
| | GAGAAGTCAACAAGCGGGTGGAAGACATCCA | | SEFEPKKKRKV* | |
| | CCCCACCGTGCCCAACCCTTACAACCTCTTG | | | |
| | AGCGGGCTCCCACCGTCCCACCAGTGGTACA | | | |
| | CTGTGCTTGATTTAAAGGATGCCTTTTTCTG | | | |
| | CCTGAGACTCCACCCCACCAGTCAGCCTCTC | | | |
| | TTCGCCTTTGAGTGGAGAGATCCAGAGATGG | | | |
| | GAATCTCAGGACAATTGACCTGGACCAGACT | | | |
| | CCCACAGGGTTTCAAAAACAGTCCCACCCTG | | | |
| | TTTAATGAGGCACTGCACAGAGACCTAGCAG | | | |
| | ACTTCCGGATCCAGCACCCAGACTTGATCCT | | | |
| | GCTACAGTACGTGGATGACTTACTGCTGGCC | | | |
| | GCCACTTCTGAGCTAGACTGCCAACAAGGTA | | | |
| | CTCGGGCCCTGTTACAAACCCTAGGGAACCT | | | |
| | CGGGTATCGGGCCTCGGCCAAGAAAGCCCAA | | | |
| | ATTTGCCAGAAACAGGTCAAGTATCTGGGGT | | | |
| | ATCTTCTAAAAGAGGGTCAGAGATGGCTGAC | | | |
| | TGAGGCCAGAAAAGAGACTGTGATGGGGCAG | | | |
| | CCTACTCCGAAGACCCCTCGACAACTAAGGG | | | |
| | AGTTCCTAGGGAAGGCAGGCTTCTGTCGCCT | | | |
| | CTTCATCCCTGGGTTTGCAGAAATGGCAGCC | | | |
| | CCCCTGTACCCTCTCACCAAACCGGGGACTC | | | |
| | TGTTTAATTGGGGCCCAGACCAACAAAAGGC | | | |
| | CTATCAAGAAATCAAGCAAGCTCTTCTAACT | | | |
| | GCCCCAGCCCTGGGGTTGCCAGATTCTGGCG | | | |
| | GCTCAAAAAGAACCGCCGACGGCAGCGAATT | | | |
| | CGAGCCCAAGAAGAAGAGGAAAGTCTAA | | | |
| bpNLS-MMLV | ATGAAACGGACAGCCGACGGAAGCGAGTTCG | 58 | MKRTADGSEFESPKKKRKVILNIEDEYRLH | 59 |
| RT | AGTCACCAAAGAAGAAGCGGAAAGTCACCCT | | ETSKEPDVSLGSTWLSDFPQAWAETGGMGL | |
| (181 AA | AAATATAGAAGATGAGTATCGGCTACATGAG | | AVRQAPLIIPLKATSTPVSIKQYPMSQEAR | |
| truncation)- | ACCTCAAAAGAGCCAGATGTTTCTCTAGGGT | | LGIKPHIQRLLDQGILVPCQSPWNTPLLPV | |
| 4 AA | CCACATGGCTGTCTGATTTTCCTCAGGCCTG | | KKPGTNDYRPVQDLREVNKRVEDIHPTVPN | |
| linker-bpNLS = | GGCGGAAACCGGGGGCATGGGACTGGCAGTT | | PYNLLSGLPPSHQWYTVLDLKDAFFCLRLH | |
| MMLV-RT(dRH) | CGCCAAGCTCCTCTGATCATACCTCTGAAAG | | PTSQPLFAFEWRDPEMGISGQLTWTRLPQG | |
| | CAACCTCTACCCCCGTGTCCATAAAACAATA | | FKNSPTLFNEALHRDLADFRIQHPDLILLQ | |
| | CCCCATGTCACAAGAAGCCAGACTGGGGATC | | YVDDLLLAATSELDCQQGTRALLQTLGNLG | |
| | AAGCCCCACATACAGAGACTGTTGGACCAGG | | YRASAKKAQICQKQVKYLGYLLKEGQRWLT | |
| | GAATACTGGTACCCTGCCAGTCCCCCTGGAA | | EARKETVMGQPTPKTPRQLREFLGKAGFCR | |
| | CACGCCCCTGCTACCCGTTAAGAAACCAGGG | | LFIPGFAEMAAPLYPLTKPGTLENWGPDQQ | |
| | ACTAATGATTATAGGCCTGTCCAGGATCTGA | | KAYQEIKQALLTAPALGLPDLTKPFELFVD | |
| | GAGAAGTCAACAAGCGGGTGGAAGACATCCA | | EKQGYAKGVLTQKLGPWRRPVAYLSKKLDP | |
| | CCCCACCGTGCCCAACCCTTACAACCTCTTG | | VAAGWPPCLRMVAAIAVLTKDAGKLTMGQP | |
| | AGCGGGCTCCCACCGTCCCACCAGTGGTACA | | LVILAPHAVEALVKQPPDRWLSNARMTHYQ | |
| | CTGTGCTTGATTTAAAGGATGCCTTTTTCTG | | ALLLDTDRVQFGPVVALNPATLLPLPEEGL | |
| | CCTGAGACTCCACCCCACCAGTCAGCCTCTC | | QHNCLSGGSKRTADGSEFEPKKKRKV* | |
| | TTCGCCTTTGAGTGGAGAGATCCAGAGATGG | | | |
| | GAATCTCAGGACAATTGACCTGGACCAGACT | | | |
| | CCCACAGGGTTTCAAAAACAGTCCCACCCTG | | | |
| | TTTAATGAGGCACTGCACAGAGACCTAGCAG | | | |
| | ACTTCCGGATCCAGCACCCAGACTTGATCCT | | | |
| | GCTACAGTACGTGGATGACTTACTGCTGGCC | | | |
| | GCCACTTCTGAGCTAGACTGCCAACAAGGTA | | | |
| | CTCGGGCCCTGTTACAAACCCTAGGGAACCT | | | |
| | CGGGTATCGGGCCTCGGCCAAGAAAGCCCAA | | | |
| | ATTTGCCAGAAACAGGTCAAGTATCTGGGGT | | | |
| | ATCTTCTAAAAGAGGGTCAGAGATGGCTGAC | | | |
| | TGAGGCCAGAAAAGAGACTGTGATGGGGCAG | | | |
| | CCTACTCCGAAGACCCCTCGACAACTAAGGG | | | |
| | AGTTCCTAGGGAAGGCAGGCTTCTGTCGCCT | | | |
| | CTTCATCCCTGGGTTTGCAGAAATGGCAGCC | | | |
| | CCCCTGTACCCTCTCACCAAACCGGGGACTC | | | |
| | TGTTTAATTGGGGCCCAGACCAACAAAAGGC | | | |
| | CTATCAAGAAATCAAGCAAGCTCTTCTAACT | | | |
| | GCCCCAGCCCTGGGGTTGCCAGATTTGACTA | | | |
| | AGCCCTTTGAACTCTTTGTCGACGAGAAGCA | | | |
| | GGGCTACGCCAAAGGTGTCCTAACGCAAAAA | | | |
| | CTGGGACCTTGGCGTCGGCCGGTGGCCTACC | | | |
| | TGTCCAAAAAGCTAGACCCAGTAGCAGCTGG | | | |
| | GTGGCCCCCTTGCCTACGGATGGTAGCAGCC | | | |
| | ATTGCCGTACTGACAAAGGATGCAGGCAAGC | | | |
| | TAACCATGGGACAGCCACTAGTCATTCTGGC | | | |
| | CCCCCATGCAGTAGAGGCACTAGTCAAACAA | | | |
| | CCCCCCGACCGCTGGCTTTCCAACGCCCGGA | | | |
| | TGACTCACTATCAGGCCTTGCTTTTGGACAC | | | |
| | GGACCGGGTCCAGTTCGGACCGGTGGTAGCC | | | |
| | CTGAACCCGGCTACGCTGCTCCCACTGCCTG | | | |
| | AGGAAGGGCTGCAACACAACTGCCTTTCTGG | | | |
| | CGGCTCAAAAAGAACCGCCGACGGCAGCGAA | | | |
| | TTCGAGCCCAAGAAGAAGAGGAAAGTCTAA | | | |
| bpNLS-MMLV | ATGAAACGGACAGCCGACGGAAGCGAGTTCG | 60 | MKRTADGSEFESPKKKRKVTWLSDFPQAWA | 61 |
| RT | AGTCACCAAAGAAGAAGCGGAAAGTCACATG | | ETGGMGLAVRQAPLIIPLKATSTPVSIKQY | |
| (23 AA + | GCTGTCTGATTTTCCTCAGGCCTGGGCGGAA | | PMSQEARLGIKPHIQRLLDQGILVPCQSPW | |
| 181 AA | ACCGGGGGCATGGGACTGGCAGTTCGCCAAG | | NTPLLPVKKPGTNDYRPVQDLREVNKRVED | |
| truncation)- | CTCCTCTGATCATACCTCTGAAAGCAACCTC | | IHPTVPNPYNLLSGLPPSHQWYTVLDLKDA | |
| 4 AA | TACCCCCGTGTCCATAAAACAATACCCCATG | | FFCLRLHPTSQPLFAFEWRDPEMGISGQLT | |
| linker-bpNLS | TCACAAGAAGCCAGACTGGGGATCAAGCCCC | | WTRLPQGFKNSPTLFNEALHRDLADFRIQH | |
| | ACATACAGAGACTGTTGGACCAGGGAATACT | | PDLILLQYVDDLLLAATSELDCQQGTRALL | |
| | GGTACCCTGCCAGTCCCCCTGGAACACGCCC | | QTLGNLGYRASAKKAQICQKQVKYLGYLLK | |
| | CTGCTACCCGTTAAGAAACCAGGGACTAATG | | EGQRWLTEARKETVMGQPTPKTPRQLREFL | |
| | ATTATAGGCCTGTCCAGGATCTGAGAGAAGT | | GKAGFCRLFIPGFAEMAAPLYPLTKPGTLF | |
| | CAACAAGCGGGTGGAAGACATCCACCCCACC | | NWGPDQQKAYQEIKQALLTAPALGLPDLTK | |
| | GTGCCCAACCCTTACAACCTCTTGAGCGGGC | | PFELFVDEKQGYAKGVLTQKLGPWRRPVAY | |
| | TCCCACCGTCCCACCAGTGGTACACTGTGCT | | LSKKLDPVAAGWPPCLRMVAAIAVLIKDAG | |
| | TGATTTAAAGGATGCCTTTTTCTGCCTGAGA | | KLTMGQPLVILAPHAVEALVKQPPDRWLSN | |
| | CTCCACCCCACCAGTCAGCCTCTCTTCGCCT | | ARMTHYQALLLDTDRVQFGPVVALNPATLL | |
| | TTGAGTGGAGAGATCCAGAGATGGGAATCTC | | PLPEEGLQHNCLSGGSKRTADGSEFEPKKK | |
| | AGGACAATTGACCTGGACCAGACTCCCACAG | | RKV* | |
| | GGTTTCAAAAACAGTCCCACCCTGTTTAATG | | | |
| | AGGCACTGCACAGAGACCTAGCAGACTTCCG | | | |
| | GATCCAGCACCCAGACTTGATCCTGCTACAG | | | |
| | TACGTGGATGACTTACTGCTGGCCGCCACTT | | | |
| | CTGAGCTAGACTGCCAACAAGGTACTCGGGC | | | |
| | CCTGTTACAAACCCTAGGGAACCTCGGGTAT | | | |
| | CGGGCCTCGGCCAAGAAAGCCCAAATTTGCC | | | |
| | AGAAACAGGTCAAGTATCTGGGGTATCTTCT | | | |
| | AAAAGAGGGTCAGAGATGGCTGACTGAGGCC | | | |
| | AGAAAAGAGACTGTGATGGGGCAGCCTACTC | | | |
| | CGAAGACCCCTCGACAACTAAGGGAGTTCCT | | | |
| | AGGGAAGGCAGGCTTCTGTCGCCTCTTCATC | | | |
| | CCTGGGTTTGCAGAAATGGCAGCCCCCCTGT | | | |
| | ACCCTCTCACCAAACCGGGGACTCTGTTTAA | | | |
| | TTGGGGCCCAGACCAACAAAAGGCCTATCAA | | | |
| | GAAATCAAGCAAGCTCTTCTAACTGCCCCAG | | | |
| | CCCTGGGGTTGCCAGATTTGACTAAGCCCTT | | | |
| | TGAACTCTTTGTCGACGAGAAGCAGGGCTAC | | | |
| | GCCAAAGGTGTCCTAACGCAAAAACTGGGAC | | | |
| | CTTGGCGTCGGCCGGTGGCCTACCTGTCCAA | | | |
| | AAAGCTAGACCCAGTAGCAGCTGGGTGGCCC | | | |
| | CCTTGCCTACGGATGGTAGCAGCCATTGCCG | | | |
| | TACTGACAAAGGATGCAGGCAAGCTAACCAT | | | |
| | GGGACAGCCACTAGTCATTCTGGCCCCCCAT | | | |
| | GCAGTAGAGGCACTAGTCAAACAACCCCCCG | | | |
| | ACCGCTGGCTTTCCAACGCCCGGATGACTCA | | | |
| | CTATCAGGCCTTGCTTTTGGACACGGACCGG | | | |
| | GTCCAGTTCGGACCGGTGGTAGCCCTGAACC | | | |
| | CGGCTACGCTGCTCCCACTGCCTGAGGAAGG | | | |
| | GCTGCAACACAACTGCCTTTCTGGCGGCTCA | | | |
| | AAAAGAACCGCCGACGGCAGCGAATTCGAGC | | | |
| | CCAAGAAGAAGAGGAAAGTCTAA | | | |
| bpNLS-MMLV | ATGAAACGGACAGCCGACGGAAGCGAGTTCG | 62 | MKRTADGSEFESPKKKRKVILNIEDEYRLH | 63 |
| RT(dRH)- | AGTCACCAAAGAAGAAGCGGAAAGTCACCCT | | ETSKEPDVSLGSTWLSDFPQAWAETGGMGL | |
| 4 AA- | AAATATAGAAGATGAGTATCGGCTACATGAG | | AVRQAPLIIPLKATSTPVSIKQYPMSQEAR | |
| bpNLS- | ACCTCAAAAGAGCCAGATGTTTCTCTAGGGT | | LGIKPHIQRLLDQGILVPCQSPWNTPLLPV | |
| P2A- | CCACATGGCTGTCTGATTTTCCTCAGGCCTG | | KKPGINDYRPVQDLREVNKRVEDIHPTVPN | |
| eGFP2394 | GGCGGAAACCGGGGGCATGGGACTGGCAGTT | | PYNLLSGLPPSHQWYTVLDLKDAFFCLRLH | |
| | CGCCAAGCTCCTCTGATCATACCTCTGAAAG | | PTSQPLFAFEWRDPEMGISGQLTWTRLPQG | |
| | CAACCTCTACCCCCGTGTCCATAAAACAATA | | FKNSPTLFNEALHRDLADFRIQHPDLILLQ | |
| | CCCCATGTCACAAGAAGCCAGACTGGGGATC | | YVDDLLLAATSELDCQQGTRALLQTLGNLG | |
| | AAGCCCCACATACAGAGACTGTTGGACCAGG | | YRASAKKAQICQKQVKYLGYLLKEGQRWLT | |
| | GAATACTGGTACCCTGCCAGTCCCCCTGGAA | | EARKETVMGQPTPKTPRQLREFLGKAGFCR | |
| | CACGCCCCTGCTACCCGTTAAGAAACCAGGG | | LFIPGFAEMAAPLYPLTKPGTLENWGPDQQ | |
| | ACTAATGATTATAGGCCTGTCCAGGATCTGA | | KAYQEIKQALLTAPALGLPDLIKPFELFVD | |
| | GAGAAGTCAACAAGCGGGTGGAAGACATCCA | | EKQGYAKGVLTQKLGPWRRPVAYLSKKLDP | |
| | CCCCACCGTGCCCAACCCTTACAACCTCTTG | | VAAGWPPCLRMVAAIAVLTKDAGKLTMGQP | |
| | AGCGGGCTCCCACCGTCCCACCAGTGGTACA | | LVILAPHAVEALVKQPPDRWLSNARMTHYQ | |
| | CTGTGCTTGATTTAAAGGATGCCTTTTTCTG | | ALLLDTDRVQFGPVVALNPATLLPLPEEGL | |
| | CCTGAGACTCCACCCCACCAGTCAGCCTCTC | | QHNCLSGGSKRTADGSEFEPKKKRKVGSGA | |
| | TTCGCCTTTGAGTGGAGAGATCCAGAGATGG | | TNFSLLKQAGDVEENPGPMVSKGEELFTGV | |
| | GAATCTCAGGACAATTGACCTGGACCAGACT | | VPILVELDGDVNGHKFSVSGEGEGDATYGK | |
| | CCCACAGGGTTTCAAAAACAGTCCCACCCTG | | LTLKFICTTGKLPVPWPTLVTILTYGVQCE | |
| | TTTAATGAGGCACTGCACAGAGACCTAGCAG | | SRYPDHMKQHDFFKSAMPEGYVQERTIFFK | |
| | ACTTCCGGATCCAGCACCCAGACTTGATCCT | | DDGNYKTRAEVKFEGDTLVNRIELKGIDEK | |
| | GCTACAGTACGTGGATGACTTACTGCTGGCC | | EDGNILGHKLEYNYNSHNVYIMADKQKNGI | |
| | GCCACTTCTGAGCTAGACTGCCAACAAGGTA | | KVNFKIRHNIEDGSVQLADHYQQNTPIGDG | |
| | CTCGGGCCCTGTTACAAACCCTAGGGAACCT | | PVLLPDNHYLSTQSALSKDPNEKRDHMVLL | |
| | CGGGTATCGGGCCTCGGCCAAGAAAGCCCAA | | EFVTAAGITLGMDELYK* | |
| | ATTTGCCAGAAACAGGTCAAGTATCTGGGGT | | | |
| | ATCTTCTAAAAGAGGGTCAGAGATGGCTGAC | | | |
| | TGAGGCCAGAAAAGAGACTGTGATGGGGCAG | | | |
| | CCTACTCCGAAGACCCCTCGACAACTAAGGG | | | |
| | AGTTCCTAGGGAAGGCAGGCTTCTGTCGCCT | | | |
| | CTTCATCCCTGGGTTTGCAGAAATGGCAGCC | | | |
| | CCCCTGTACCCTCTCACCAAACCGGGGACTC | | | |
| | TGTTTAATTGGGGCCCAGACCAACAAAAGGC | | | |
| | CTATCAAGAAATCAAGCAAGCTCTTCTAACT | | | |
| | GCCCCAGCCCTGGGGTTGCCAGATTTGACTA | | | |
| | AGCCCTTTGAACTCTTTGTCGACGAGAAGCA | | | |
| | GGGCTACGCCAAAGGTGTCCTAACGCAAAAA | | | |
| | CTGGGACCTTGGCGTCGGCCGGTGGCCTACC | | | |
| | TGTCCAAAAAGCTAGACCCAGTAGCAGCTGG | | | |
| | GTGGCCCCCTTGCCTACGGATGGTAGCAGCC | | | |
| | ATTGCCGTACTGACAAAGGATGCAGGCAAGC | | | |
| | TAACCATGGGACAGCCACTAGTCATTCTGGC | | | |
| | CCCCCATGCAGTAGAGGCACTAGTCAAACAA | | | |
| | CCCCCCGACCGCTGGCTTTCCAACGCCCGGA | | | |
| | TGACTCACTATCAGGCCTTGCTTTTGGACAC | | | |
| | GGACCGGGTCCAGTTCGGACCGGTGGTAGCC | | | |
| | CTGAACCCGGCTACGCTGCTCCCACTGCCTG | | | |
| | AGGAAGGGCTGCAACACAACTGCCTTTCTGG | | | |
| | CGGCTCAAAAAGAACCGCCGACGGCAGCGAA | | | |
| | TTCGAGCCCAAGAAGAAGAGGAAAGTCGGAA | | | |
| | GCGGAGCTACTAACTTCAGCCTGCTGAAGCA | | | |
| | GGCTGGAGACGTGGAGGAGAACCCTGGACCT | | | |
| | ATGGTGAGCAAGGGCGAGGAGCTGTTCACCG | | | |
| | GGGTGGTGCCCATCCTGGTCGAGCTGGACGG | | | |
| | CGACGTAAACGGCCACAAGTTCAGCGTGTCC | | | |
| | GGCGAGGGCGAGGGCGATGCCACCTACGGCA | | | |
| | AGCTGACCCTGAAGTTCATCTGCACCACCGG | | | |
| | CAAGCTGCCCGTGCCCTGGCCCACCCTCGTG | | | |
| | ACCACCCTGACCTATGGAGTGCAGTGCTTCA | | | |
| | GCCGCTACCCCGACCACATGAAGCAGCACGA | | | |
| | CTTCTTCAAGTCCGCCATGCCCGAAGGCTAC | | | |
| | GTCCAGGAGCGCACCATCTTCTTCAAGGACG | | | |
| | ACGGCAACTACAAGACCCGCGCCGAGGTGAA | | | |
| | GTTCGAGGGCGACACCCTGGTGAACCGCATC | | | |
| | GAGCTGAAGGGCATCGACTTCAAGGAGGACG | | | |
| | GCAACATCCTGGGGCACAAGCTGGAGTACAA | | | |
| | CTACAACAGCCACAACGTCTATATCATGGCC | | | |
| | GACAAGCAGAAGAACGGCATCAAGGTGAACT | | | |
| | TCAAGATCCGCCACAACATCGAGGACGGCAG | | | |
| | CGTGCAGCTCGCCGACCACTACCAGCAGAAC | | | |
| | ACCCCCATCGGCGACGGCCCCGTGCTGCTGC | | | |
| | CCGACAACCACTACCTGAGCACCCAGTCCGC | | | |
| | CCTGAGCAAAGACCCCAACGAGAAGCGCGAT | | | |
| | CACATGGTCCTGCTGGAGTTCGTGACCGCCG | | | |
| | CCGGGATCACTCTCGGCATGGACGAGCTGTA | | | |
| | CAAGTAA | | | |
| bpNLS-nCas9 | ATGAAACGGACAGCCGACGGAAGCGAGTTCG | 64 | MKRTADGSEFESPKKKRKVDKKYSIGLDIG | 65 |
| (H840A)- | AGTCACCAAAGAAGAAGCGGAAAGTCGACAA | | TNSVGWAVITDEYKVPSKKFKVLGNTDRHS | |
| P2A-MMLV | GAAGTACAGCATCGGCCTGGACATCGGCACC | | IKKNLIGALLFDSGETAEATRLKRTARRRY | |
| RT | AACTCTGTGGGCTGGGCCGTGATCACCGACG | | TRRKNRICYLQEIFSNEMAKVDDSFFHRLE | |
| (dRH)- | AGTACAAGGTGCCCAGCAAGAAATTCAAGGT | | ESFLVEEDKKHERHPIFGNIVDEVAYHEKY | |
| 4 AA | GCTGGGCAACACCGACCGGCACAGCATCAAG | | PTIYHLRKKLVDSTDKADLRLIYLALAHMI | |
| linker- | AAGAACCTGATCGGAGCCCTGCTGTTCGACA | | KERGHFLIEGDLNPDNSDVDKLFIQLVQTY | |
| bpNLS | GCGGCGAAACAGCCGAGGCCACCCGGCTGAA | | NQLFEENPINASGVDAKAILSARLSKSRRL | |
| | GAGAACCGCCAGAAGAAGATACACCAGACGG | | ENLIAQLPGEKKNGLFGNLIALSLGLIPNF | |
| | AAGAACCGGATCTGCTATCTGCAAGAGATCT | | KSNFDLAEDAKLQLSKDTYDDDLQNLLAQI | |
| | TCAGCAACGAGATGGCCAAGGTGGACGACAG | | GDQYADLFLAAKNLSDAILLSDILRVNTEI | |
| | CTTCTTCCACAGACTGGAAGAGTCCTTCCTG | | TKAPLSASMIKRYDEHHQDLTLLKALVRQQ | |
| | GTGGAAGAGGATAAGAAGCACGAGCGGCACC | | LPEKYKEIFFDQSKNGYAGYIDGGASQEEF | |
| | CCATCTTCGGCAACATCGTGGACGAGGTGGC | | YKFIKPILEKMDGTEELLVKLNREDLLRKQ | |
| | CTACCACGAGAAGTACCCCACCATCTACCAC | | RTFDNGSIPHQIHLGELHAILRRQEDFYPF | |
| | CTGAGAAAGAAACTGGTGGACAGCACCGACA | | LKDNREKIEKILTFRIPYYVGPLARGNSRF | |
| | AGGCCGACCTGCGGCTGATCTATCTGGCCCT | | AWMTRKSEETITPWNFEEVVDKGASAQSFI | |
| | GGCCCACATGATCAAGTTCCGGGGCCACTTC | | ERMTNFDKNLPNEKVLPKHSLLYEYFTVYN | |
| | CTGATCGAGGGCGACCTGAACCCCGACAACA | | ELTKVKYVTEGMRKPAFLSGEQKKAIVDLL | |
| | GCGACGTGGACAAGCTGTTCATCCAGCTGGT | | FKTNRKVTVKQLKEDYFKKIECFDSVEISG | |
| | GCAGACCTACAACCAGCTGTTCGAGGAAAAC | | VEDRFNASLGTYHDLLKIIKDKDFLDNEEN | |
| | CCCATCAACGCCAGCGGCGTGGACGCCAAGG | | EDILEDIVETLTLFEDREMIEERLKTYAHL | |
| | CCATCCTGTCTGCCAGACTGAGCAAGAGCAG | | FDDKVMKQLKRRRYTGWGRLSRKLINGIRD | |
| | ACGGCTGGAAAATCTGATCGCCCAGCTGCCC | | KQSGKTILDFLKSDGFANRNFMQLIHDDSL | |
| | GGCGAGAAGAAGAATGGCCTGTTCGGAAACC | | TFKEDIQKAQVSGQGDSLHEHIANLAGSPA | |
| | TGATTGCCCTGAGCCTGGGCCTGACCCCCAA | | IKKGILQTVKVVDELVKVMGRHKPENIVIE | |
| | CTTCAAGAGCAACTTCGACCTGGCCGAGGAT | | MARENQTTQKGQKNSRERMKRIEEGIKELG | |
| | GCCAAACTGCAGCTGAGCAAGGACACCTACG | | SQILKEHPVENTQLQNEKLYLYYLQNGRDM | |
| | ACGACGACCTGGACAACCTGCTGGCCCAGAT | | YVDQELDINRLSDYDVDAIVPQSFLKDDSI | |
| | CGGCGACCAGTACGCCGACCTGTTTCTGGCC | | DNKVLTRSDKNRGKSDNVPSEEVVKKMKNY | |
| | GCCAAGAACCTGTCCGACGCCATCCTGCTGA | | WRQLLNAKLITQRKEDNLTKAERGGLSELD | |
| | GCGACATCCTGAGAGTGAACACCGAGATCAC | | KAGFIKRQLVETRQITKHVAQILDSRMNTK | |
| | CAAGGCCCCCCTGAGCGCCTCTATGATCAAG | | YDENDKLIREVKVITLKSKLVSDFRKDEQF | |
| | AGATACGACGAGCACCACCAGGACCTGACCC | | YKVREINNYHHAHDAYLNAVVGTALIKKYP | |
| | TGCTGAAAGCTCTCGTGCGGCAGCAGCTGCC | | KLESEFVYGDYKVYDVRKMIAKSEQEIGKA | |
| | TGAGAAGTACAAAGAGATTTTCTTCGACCAG | | TAKYFFYSNIMNFFKTEITLANGEIRKRPL | |
| | AGCAAGAACGGCTACGCCGGCTACATTGACG | | IETNGETGEIVWDKGRDFATVRKVLSMPQV | |
| | GCGGAGCCAGCCAGGAAGAGTTCTACAAGTT | | NIVKKTEVQTGGFSKESILPKRNSDKLIAR | |
| | CATCAAGCCCATCCTGGAAAAGATGGACGGC | | KKDWDPKKYGGFDSPTVAYSVLVVAKVEKG | |
| | ACCGAGGAACTGCTCGTGAAGCTGAACAGAG | | KSKKLKSVKELLGITIMERSSFEKNPIDFL | |
| | AGGACCTGCTGCGGAAGCAGCGGACCTTCGA | | EAKGYKEVKKDLIIKLPKYSLFELENGRKR | |
| | CAACGGCAGCATCCCCCACCAGATCCACCTG | | MLASAGELQKGNELALPSKYVNFLYLASHY | |
| | GGAGAGCTGCACGCCATTCTGCGGCGGCAGG | | EKLKGSPEDNEQKQLFVEQHKHYLDEIIEQ | |
| | AAGATTTTTACCCATTCCTGAAGGACAACCG | | ISEFSKRVILADANLDKVLSAYNKHRDKPI | |
| | GGAAAAGATCGAGAAGATCCTGACCTTCCGC | | REQAENIIHLFTLINLGAPAAFKYFDTTID | |
| | ATCCCCTACTACGTGGGCCCTCTGGCCAGGG | | RKRYTSTKEVLDATLIHQSITGLYETRIDL | |
| | GAAACAGCAGATTCGCCTGGATGACCAGAAA | | SQLGGDATNFSLLKQAGDVEENPGPTLNIE | |
| | GAGCGAGGAAACCATCACCCCCTGGAACTTC | | DEYRLHETSKEPDVSLGSTWLSDFPQAWAE | |
| | GAGGAAGTGGTGGACAAGGGCGCTTCCGCCC | | TGGMGLAVRQAPLIIPLKATSTPVSIKQYP | |
| | AGAGCTTCATCGAGCGGATGACCAACTTCGA | | MSQEARLGIKPHIQRLLDQGILVPCQSPWN | |
| | TAAGAACCTGCCCAACGAGAAGGTGCTGCCC | | TPLLPVKKPGINDYRPVQDLREVNKRVEDI | |
| | AAGCACAGCCTGCTGTACGAGTACTTCACCG | | HPTVPNPYNLLSGLPPSHQWYTVLDLKDAF | |
| | TGTATAACGAGCTGACCAAAGTGAAATACGT | | FCLRLHPTSQPLFAFEWRDPEMGISGQLTW | |
| | GACCGAGGGAATGAGAAAGCCCGCCTTCCTG | | TRLPQGFKNSPTLFNEALHRDLADFRIQHP | |
| | AGCGGCGAGCAGAAAAAGGCCATCGTGGACC | | DLILLQYVDDLLLAATSELDCQQGTRALLQ | |
| | TGCTGTTCAAGACCAACCGGAAAGTGACCGT | | TLGNLGYRASAKKAQICQKQVKYLGYLLKE | |
| | GAAGCAGCTGAAAGAGGACTACTTCAAGAAA | | GQRWLTEARKETVMGQPTPKTPRQLREFLG | |
| | ATCGAGTGCTTCGACTCCGTGGAAATCTCCG | | KAGFCRLFIPGFAEMAAPLYPLTKPGTLEN | |
| | GCGTGGAAGATCGGTTCAACGCCTCCCTGGG | | WGPDQQKAYQEIKQALLTAPALGLPDLTKP | |
| | CACATACCACGATCTGCTGAAAATTATCAAG | | FELFVDEKQGYAKGVLTQKLGPWRRPVAYL | |
| | GACAAGGACTTCCTGGACAATGAGGAAAACG | | SKKLDPVAAGWPPCLRMVAAIAVLTKDAGK | |
| | AGGACATTCTGGAAGATATCGTGCTGACCCT | | LTMGQPLVILAPHAVEALVKQPPDRWLSNA | |
| | GACACTGTTTGAGGACAGAGAGATGATCGAG | | RMTHYQALLLDTDRVQFGPVVALNPATLLP | |
| | GAACGGCTGAAAACCTATGCCCACCTGTTCG | | LPEEGLQHNCLSGGSKRTADGSEFEPKKKR | |
| | ACGACAAAGTGATGAAGCAGCTGAAGCGGCG | | KV* | |
| | GAGATACACCGGCTGGGGCAGGCTGAGCCGG | | | |
| | AAGCTGATCAACGGCATCCGGGACAAGCAGT | | | |
| | CCGGCAAGACAATCCTGGATTTCCTGAAGTC | | | |
| | CGACGGCTTCGCCAACAGAAACTTCATGCAG | | | |
| | CTGATCCACGACGACAGCCTGACCTTTAAAG | | | |
| | AGGACATCCAGAAAGCCCAGGTGTCCGGCCA | | | |
| | GGGCGATAGCCTGCACGAGCACATTGCCAAT | | | |
| | CTGGCCGGCAGCCCCGCCATTAAGAAGGGCA | | | |
| | TCCTGCAGACAGTGAAGGTGGTGGACGAGCT | | | |
| | CGTGAAAGTGATGGGCCGGCACAAGCCCGAG | | | |
| | AACATCGTGATCGAAATGGCCAGAGAGAACC | | | |
| | AGACCACCCAGAAGGGACAGAAGAACAGCCG | | | |
| | CGAGAGAATGAAGCGGATCGAAGAGGGCATC | | | |
| | AAAGAGCTGGGCAGCCAGATCCTGAAAGAAC | | | |
| | ACCCCGTGGAAAACACCCAGCTGCAGAACGA | | | |
| | GAAGCTGTACCTGTACTACCTGCAGAATGGG | | | |
| | CGGGATATGTACGTGGACCAGGAACTGGACA | | | |
| | TCAACCGGCTGTCCGACTACGATGTGGACGC | | | |
| | TATCGTGCCTCAGAGCTTTCTGAAGGACGAC | | | |
| | TCCATCGACAACAAGGTGCTGACCAGAAGCG | | | |
| | ACAAGAACCGGGGCAAGAGCGACAACGTGCC | | | |
| | CTCCGAAGAGGTCGTGAAGAAGATGAAGAAC | | | |
| | TACTGGCGGCAGCTGCTGAACCCCAAGCTGA | | | |
| | TTACCCAGAGAAAGTTCGACAATCTGACCAA | | | |
| | GGCCGAGAGAGGCGGCCTGAGCGAACTGGAT | | | |
| | AAGGCCGGCTTCATCAAGAGACAGCTGGTGG | | | |
| | AAACCCGGCAGATCACAAAGCACGTGGCACA | | | |
| | GATCCTGGACTCCCGGATGAACACTAAGTAC | | | |
| | GACGAGAATGACAAGCTGATCCGGGAAGTGA | | | |
| | AAGTGATCACCCTGAAGTCCAAGCTGGTGTC | | | |
| | CGATTTCCGGAAGGATTTCCAGTTTTACAAA | | | |
| | GTGCGCGAGATCAACAACTACCACCACGCCC | | | |
| | ACGACGCCTACCTGAACGCCGTCGTGGGAAC | | | |
| | CGCCCTGATCAAAAAGTACCCTAAGCTGGAA | | | |
| | AGCGAGTTCGTGTACGGCGACTACAAGGTGT | | | |
| | ACGACGTGCGGAAGATGATCGCCAAGAGCGA | | | |
| | GCAGGAAATCGGCAAGGCTACCGCCAAGTAC | | | |
| | TTCTTCTACAGCAACATCATGAACTTTTTCA | | | |
| | AGACCGAGATTACCCTGGCCAACGGCGAGAT | | | |
| | CCGGAAGCGGCCTCTGATCGAGACAAACGGC | | | |
| | GAAACCGGGGAGATCGTGTGGGATAAGGGCC | | | |
| | GGGATTTTGCCACCGTGCGGAAAGTGCTGAG | | | |
| | CATGCCCCAAGTGAATATCGTGAAAAAGACC | | | |
| | GAGGTGCAGACAGGCGGCTTCAGCAAAGAGT | | | |
| | CTATCCTGCCCAAGAGGAACAGCGATAAGCT | | | |
| | GATCGCCAGAAAGAAGGACTGGGACCCTAAG | | | |
| | AAGTACGGCGGCTTCGACAGCCCCACCGTGG | | | |
| | CCTATTCTGTGCTGGTGGTGGCCAAAGTGGA | | | |
| | AAAGGGCAAGTCCAAGAAACTGAAGAGTGTG | | | |
| | AAAGAGCTGCTGGGGATCACCATCATGGAAA | | | |
| | GAAGCAGCTTCGAGAAGAATCCCATCGACTT | | | |
| | TCTGGAAGCCAAGGGCTACAAAGAAGTGAAA | | | |
| | AAGGACCTGATCATCAAGCTGCCTAAGTACT | | | |
| | CCCTGTTCGAGCTGGAAAACGGCCGGAAGAG | | | |
| | AATGCTGGCCTCTGCCGGCGAACTGCAGAAG | | | |
| | GAGATACACCGGCTGGGGCAGGCTGAGCCGG | | | |
| | AAGCTGATCAACGGCATCCGGGACAAGCAGT | | | |
| | CCGGCAAGACAATCCTGGATTTCCTGAAGTC | | | |
| | CGACGGCTTCGCCAACAGAAACTTCATGCAG | | | |
| | CTGATCCACGACGACAGCCTGACCTTTAAAG | | | |
| | AGGACATCCAGAAAGCCCAGGTGTCCGGCCA | | | |
| | GGGCGATAGCCTGCACGAGCACATTGCCAAT | | | |
| | CTGGCCGGCAGCCCCGCCATTAAGAAGGGCA | | | |
| | TCCTGCAGACAGTGAAGGTGGTGGACGAGCT | | | |
| | CGTGAAAGTGATGGGCCGGCACAAGCCCGAG | | | |
| | AACATCGTGATCGAAATGGCCAGAGAGAACC | | | |
| | AGACCACCCAGAAGGGACAGAAGAACAGCCG | | | |
| | CGAGAGAATGAAGCGGATCGAAGAGGGCATC | | | |
| | AAAGAGCTGGGCAGCCAGATCCTGAAAGAAC | | | |
| | ACCCCGTGGAAAACACCCAGCTGCAGAACGA | | | |
| | GAAGCTGTACCTGTACTACCTGCAGAATGGG | | | |
| | CGGGATATGTACGTGGACCAGGAACTGGACA | | | |
| | TCAACCGGCTGTCCGACTACGATGTGGACGC | | | |
| | TATCGTGCCTCAGAGCTTTCTGAAGGACGAC | | | |
| | TCCATCGACAACAAGGTGCTGACCAGAAGCG | | | |
| | ACAAGAACCGGGGCAAGAGCGACAACGTGCC | | | |
| | CTCCGAAGAGGTCGTGAAGAAGATGAAGAAC | | | |
| | TACTGGCGGCAGCTGCTGAACGCCAAGCTGA | | | |
| | TTACCCAGAGAAAGTTCGACAATCTGACCAA | | | |
| | GGCCGAGAGAGGCGGCCTGAGCGAACTGGAT | | | |
| | AAGGCCGGCTTCATCAAGAGACAGCTGGTGG | | | |
| | AAACCCGGCAGATCACAAAGCACGTGGCACA | | | |
| | GATCCTGGACTCCCGGATGAACACTAAGTAC | | | |
| | GACGAGAATGACAAGCTGATCCGGGAAGTGA | | | |
| | AAGTGATCACCCTGAAGTCCAAGCTGGTGTC | | | |
| | CGATTTCCGGAAGGATTTCCAGTTTTACAAA | | | |
| | GTGCGCGAGATCAACAACTACCACCACGCCC | | | |
| | ACGACGCCTACCTGAACGCCGTCGTGGGAAC | | | |
| | CGCCCTGATCAAAAAGTACCCTAAGCTGGAA | | | |
| | AGCGAGTTCGTGTACGGCGACTACAAGGTGT | | | |
| | ACGACGTGCGGAAGATGATCGCCAAGAGCGA | | | |
| | GCAGGAAATCGGCAAGGCTACCGCCAAGTAC | | | |
| | TTCTTCTACAGCAACATCATGAACTTTTTCA | | | |
| | AGACCGAGATTACCCTGGCCAACGGCGAGAT | | | |
| | CCGGAAGCGGCCTCTGATCGAGACAAACGGC | | | |
| | GAAACCGGGGAGATCGTGTGGGATAAGGGCC | | | |
| | GGGATTTTGCCACCGTGCGGAAAGTGCTGAG | | | |
| | CATGCCCCAAGTGAATATCGTGAAAAAGACC | | | |
| | GAGGTGCAGACAGGCGGCTTCAGCAAAGAGT | | | |
| | CTATCCTGCCCAAGAGGAACAGCGATAAGCT | | | |
| | GATCGCCAGAAAGAAGGACTGGGACCCTAAG | | | |
| | AAGTACGGCGGCTTCGACAGCCCCACCGTGG | | | |
| | CCTATTCTGTGCTGGTGGTGGCCAAAGTGGA | | | |
| | AAAGGGCAAGTCCAAGAAACTGAAGAGTGTG | | | |
| | AAAGAGCTGCTGGGGATCACCATCATGGAAA | | | |
| | GAAGCAGCTTCGAGAAGAATCCCATCGACTT | | | |
| | TCTGGAAGCCAAGGGCTACAAAGAAGTGAAA | | | |
| | AAGGACCTGATCATCAAGCTGCCTAAGTACT | | | |
| | CCCTGTTCGAGCTGGAAAACGGCCGGAAGAG | | | |
| | AATGCTGGCCTCTGCCGGCGAACTGCAGAAG | | | |
| | GGAAACGAACTGGCCCTGCCCTCCAAATATG | | | |
| | TGAACTTCCTGTACCTGGCCAGCCACTATGA | | | |
| | GAAGCTGAAGGGCTCCCCCGAGGATAATGAG | | | |
| | CAGAAACAGCTGTTTGTGGAACAGCACAAGC | | | |
| | ACTACCTGGACGAGATCATCGAGCAGATCAG | | | |
| | CGAGTTCTCCAAGAGAGTGATCCTGGCCGAC | | | |
| | GCTAATCTGGACAAAGTGCTGTCCGCCTACA | | | |
| | ACAAGCACCGGGATAAGCCCATCAGAGAGCA | | | |
| | GGCCGAGAATATCATCCACCTGTTTACCCTG | | | |
| | ACCAATCTGGGAGCCCCTGCCGCCTTCAAGT | | | |
| | ACTTTGACACCACCATCGACCGGAAGAGGTA | | | |
| | CACCAGCACCAAAGAGGTGCTGGACGCCACC | | | |
| | CTGATCCACCAGAGCATCACCGGCCTGTACG | | | |
| | AGACACGGATCGACCTGTCTCAGCTGGGAGG | | | |
| | TGACGCTACTAACTTCAGCCTGCTGAAGCAG | | | |
| | GCTGGAGACGTGGAGGAGAACCCTGGACCTA | | | |
| | CCCTAAATATAGAAGATGAGTATCGGCTACA | | | |
| | TGAGACCTCAAAAGAGCCAGATGTTTCTCTA | | | |
| | GGGTCCACATGGCTGTCTGATTTTCCTCAGG | | | |
| | CCTGGGCGGAAACCGGGGGCATGGGACTGGC | | | |
| | AGTTCGCCAAGCTCCTCTGATCATACCTCTG | | | |
| | AAAGCAACCTCTACCCCCGTGTCCATAAAAC | | | |
| | AATACCCCATGTCACAAGAAGCCAGACTGGG | | | |
| | GATCAAGCCCCACATACAGAGACTGTTGGAC | | | |
| | CAGGGAATACTGGTACCCTGCCAGTCCCCCT | | | |
| | GGAACACGCCCCTGCTACCCGTTAAGAAACC | | | |
| | AGGGACTAATGATTATAGGCCTGTCCAGGAT | | | |
| | CTGAGAGAAGTCAACAAGCGGGTGGAAGACA | | | |
| | TCCACCCCACCGTGCCCAACCCTTACAACCT | | | |
| | CTTGAGCGGGCTCCCACCGTCCCACCAGTGG | | | |
| | TACACTGTGCTTGATTTAAAGGATGCCTTTT | | | |
| | TCTGCCTGAGACTCCACCCCACCAGTCAGCC | | | |
| | TCTCTTCGCCTTTGAGTGGAGAGATCCAGAG | | | |
| | ATGGGAATCTCAGGACAATTGACCTGGACCA | | | |
| | GACTCCCACAGGGTTTCAAAAACAGTCCCAC | | | |
| | CCTGTTTAATGAGGCACTGCACAGAGACCTA | | | |
| | GCAGACTTCCGGATCCAGCACCCAGACTTGA | | | |
| | TCCTGCTACAGTACGTGGATGACTTACTGCT | | | |
| | GGCCGCCACTTCTGAGCTAGACTGCCAACAA | | | |
| | GGTACTCGGGCCCTGTTACAAACCCTAGGGA | | | |
| | ACCTCGGGTATCGGGCCTCGGCCAAGAAAGC | | | |
| | CCAAATTTGCCAGAAACAGGTCAAGTATCTG | | | |
| | GGGTATCTTCTAAAAGAGGGTCAGAGATGGC | | | |
| | TGACTGAGGCCAGAAAAGAGACTGTGATGGG | | | |
| | GCAGCCTACTCCGAAGACCCCTCGACAACTA | | | |
| | AGGGAGTTCCTAGGGAAGGCAGGCTTCTGTC | | | |
| | GCCTCTTCATCCCTGGGTTTGCAGAAATGGC | | | |
| | AGCCCCCCTGTACCCTCTCACCAAACCGGGG | | | |
| | ACTCTGTTTAATTGGGGCCCAGACCAACAAA | | | |
| | AGGCCTATCAAGAAATCAAGCAAGCTCTTCT | | | |
| | AACTGCCCCAGCCCTGGGGTTGCCAGATTTG | | | |
| | ACTAAGCCCTTTGAACTCTTTGTCGACGAGA | | | |
| | AGCAGGGCTACGCCAAAGGTGTCCTAACGCA | | | |
| | AAAACTGGGACCTTGGCGTCGGCCGGTGGCC | | | |
| | TACCTGTCCAAAAAGCTAGACCCAGTAGCAG | | | |
| | CTGGGTGGCCCCCTTGCCTACGGATGGTAGC | | | |
| | AGCCATTGCCGTACTGACAAAGGATGCAGGC | | | |
| | AAGCTAACCATGGGACAGCCACTAGTCATTC | | | |
| | TGGCCCCCCATGCAGTAGAGGCACTAGTCAA | | | |
| | ACAACCCCCCGACCGCTGGCTTTCCAACGCC | | | |
| | CGGATGACTCACTATCAGGCCTTGCTTTTGG | | | |
| | ACACGGACCGGGTCCAGTTCGGACCGGTGGT | | | |
| | AGCCCTGAACCCGGCTACGCTGCTCCCACTG | | | |
| | CCTGAGGAAGGGCTGCAACACAACTGCCTTT | | | |
| | CTGGCGGCTCAAAAAGAACCGCCGACGGCAG | | | |
| | CGAATTCGAGCCCAAGAAGAAGAGGAAAGTC | | | |
| | TAA | | | |
| bpNLS-HFV | ATGAAACGGACAGCCGACGGAAGCGAGTTCG | 66 | MKRTADGSEFESPKKKRKVNQVGHRKIRPH | 67 |
| RT-4 AA | AGTCACCAAAGAAGAAGCGGAAAGTCAACCA | | HIATGDYPPRPQKQYPINPKAKPSIQIVID | |
| linker- | GGTGGGCCACAGAAAGATCCGCCCTCACAAC | | DLLKQGVLTPQNSTMNTPVYPVPKPDGRWR | |
| bpNLS | ATCGCCACCGGAGATTACCCCCCCAGACCTC | | MVLDYREVNKTIPLTAAQNQHSAGILATIV | |
| | AGAAACAGTATCCTATTAACCCCAAGGCCAA | | RQKYKTTLDLANGFWAHPITPESYWLTAFT | |
| | GCCCAGCATCCAGATCGTTATCGACGACCTG | | WQGKQYCWTRLPQGFLNSPALFTADVVDLL | |
| | CTTAAACAGGGCGTGCTGACCCCTCAGAACA | | KEIPNVQVYVDDIYLSHDDPKEHVQQLEKV | |
| | GCACCATGAACACCCCTGTATATCCTGTGCC | | FQILLQAGYVVSLKKSEIGQKTVEFLGENI | |
| | TAAGCCTGATGGCAGGTGGCGGATGGTCCTG | | TKEGRGLTDTFKTKLLNITPPKDLKQLQSI | |
| | GACTACAGAGAGGTGAACAAGACTATTCCCC | | LGLLNPARNFIPNFAELVQPLYNLIASAKG | |
| | TGACCGCAGCCCAGAACCAGCACAGCGCCGG | | KYIEWSEENTKQLNMVIEALNTASNLEERL | |
| | CATCCTGGCCACAATCGTGCGGCAGAAGTAC | | PEQRLVIKVNTSPSAGYVRYYNETGKKPIM | |
| | AAGACAACCCTGGATCTGGCTAATGGCTTCT | | YLNYVFSKAELKFSMLEKLLTTMHKALIKA | |
| | GGGCCCACCCCATCACACCAGAAAGCTACTG | | MDLAMGQEILVYSPIVSMTKIQKTPLPERK | |
| | GCTGACAGCTTTTACCTGGCAGGGCAAGCAG | | ALPIRWITWMTYLEDPRIQFHYDKTLPELK | |
| | TACTGCTGGACCAGACTGCCCCAGGGCTTCC | | HIPDVYTSSQSPVKHPSQYEGVFYTDGSAI | |
| | TGAATTCTCCTGCCCTGTTCACCGCTGATGT | | KSPDPTKSNNAGMGIVHATYKPEYQVLNQW | |
| | GGTGGACCTGCTGAAAGAAATCCCCAATGTG | | SIPLGNHTAQMAEIAAVEFACKKALKIPGP | |
| | CAGGTGTACGTGGATGACATCTACCTGAGCC | | VLVITDSFYVAESANKELPYWKSNGFVNNK | |
| | ACGACGACCCTAAAGAGCACGTGCAGCAGCT | | KKPLKHISKWKSIAECLSMKPDITIQHEKG | |
| | GGAAAAGGTGTTTCAGATCCTGCTGCAGGCC | | ISLQIPVFILKGNALADKLATQGSYVVNSG | |
| | GGCTACGTGGTGAGCCTGAAGAAAAGCGAGA | | GSKRTADGSEFEPKKKRKV* | |
| | TAGGACAGAAGACCGTGGAATTCCTGGGATT | | | |
| | TAACATCACAAAAGAGGGCCGGGGCCTGACA | | | |
| | GACACCTTCAAGACCAAGCTGCTGAACATCA | | | |
| | CTCCCCCCAAGGACCTGAAACAACTGCAATC | | | |
| | TATTCTGGGCCTGCTGAATTTCGCCAGAAAC | | | |
| | TTCATCCCTAACTTCGCCGAGCTGGTGCAAC | | | |
| | CTCTTTATAACCTGATCGCCTCCGCCAAGGG | | | |
| | AAAGTACATCGAGTGGAGCGAGGAAAACACA | | | |
| | AAGCAGCTGAACATGGTGATCGAGGCCCTGA | | | |
| | ACACCGCTTCTAATCTGGAAGAGCGGCTGCC | | | |
| | AGAGCAGAGACTGGTGATCAAGGTGAACACC | | | |
| | AGCCCCAGCGCTGGCTACGTGCGGTACTACA | | | |
| | ACGAGACAGGCAAGAAACCTATCATGTACCT | | | |
| | GAACTACGTGTTCAGCAAGGCTGAACTCAAG | | | |
| | TTCAGCATGCTGGAAAAACTGCTGACCACCA | | | |
| | TGCACAAGGCCCTCATCAAGGCCATGGACCT | | | |
| | GGCTATGGGACAGGAGATCCTGGTGTACAGC | | | |
| | CCAATCGTGTCCATGACCAAGATCCAAAAAA | | | |
| | CACCTCTGCCCGAAAGAAAGGCTCTGCCTAT | | | |
| | CAGATGGATCACCTGGATGACCTACCTGGAA | | | |
| | GATCCTAGAATCCAGTTCCACTACGACAAGA | | | |
| | CCCTGCCTGAGCTGAAACATATCCCAGACGT | | | |
| | GTACACCTCTAGCCAGAGCCCTGTCAAGCAT | | | |
| | CCTAGCCAGTACGAGGGCGTTTTCTACACAG | | | |
| | ACGGCAGCGCCATCAAGAGCCCTGATCCTAC | | | |
| | AAAGTCCAACAACGCTGGCATGGGCATCGTG | | | |
| | CACGCCACATACAAGCCCGAGTACCAGGTGC | | | |
| | TGAATCAGTGGTCCATCCCTCTGGGCAACCA | | | |
| | CACCGCCCAAATGGCCGAAATCGCCGCCGTG | | | |
| | GAATTCGCCTGCAAGAAGGCGCTGAAGATCC | | | |
| | CAGGCCCTGTGCTGGTCATTACAGATAGCTT | | | |
| | CTACGTGGCCGAGAGCGCCAACAAGGAGCTG | | | |
| | CCCTACTGGAAGTCTAACGGCTTTGTGAACA | | | |
| | ACAAGAAGAAGCCTCTGAAGCACATCTCCAA | | | |
| | GTGGAAATCTATCGCCGAGTGTCTGTCTATG | | | |
| | AAGCCTGACATCACCATCCAGCACGAGAAGG | | | |
| | GCATCAGCCTGCAGATCCCTGTGTTCATCCT | | | |
| | GAAGGGCAACGCCCTGGCCGACAAGCTGGCC | | | |
| | ACCCAGGGCAGCTATGTGGTCAATTCTGGCG | | | |
| | GCTCAAAAAGAACCGCCGACGGCAGCGAATT | | | |
| | CGAGCCCAAGAAGAAGAGGAAAGTCTAA | | | |
| bpNLS-HERV- | ATGAAACGGACAGCCGACGGAAGCGAGTTCG | 68 | MKRTADGSEFESPKKKRKVKPTMAILERIS | 69 |
| Kcon | AGTCACCAAAGAAGAAGCGGAAAGTCAAAAG | | KNSQENIDEVFTRLYRYLLRPDIYYVAYQN | |
| RT-4 AA | CAGAAAACGGAGAAATAGAGTGTCCTTCCTG | | LYSNKGASTKGILDDTADGFSEEKIKKIIQ | |
| linker- | GGCGCTGCCACAGTGGAACCACCTAAGCCCA | | SLKDGTYYPQPVRRMYIAKKNSKKMRPLGI | |
| bpNLS | TCCCTCTGACATGGAAAACAGAGAAGCCTGT | | PTFTDKLIQEAVRIILESIYEPVFEDVSHG | |
| | GTGGGTCAACCAGTGGCCTCTGCCTAAGCAG | | FRPQRSCHTALKTIKREFGGARWFVEGDIK | |
| | AAGCTGGAGGCTCTCCACCTGCTGGCCAACG | | GCFDNIDHVTLIGLINLKIKDMKMSQLIYK | |
| | AGCAGCTTGAGAAGGGCCACATCGAGCCCAG | | FLKAGYLENWQYHKTYSGTPQGGILSPLLA | |
| | CTTTAGCCCTTGGAACAGCCCTGTGTTCGTG | | NIYLHELDKFVLQLKMKFDRESPERITPEY | |
| | ATCCAGAAGAAGAGCGGCAAGTGGCGGATGC | | RELHNEIKRISHRLKKLEGEEKAKVLLEYQ | |
| | TGACAGATCTGAGAGCTGTGAACGCCGTGAT | | EKRKRLPTLPCTSQINKVLKYVRYADDFII | |
| | CCAACCCATGGGCCCCCTGCAGCCAGGCCTG | | SVKGSKEDCQWIKEQLKLFIHNKLKMELSE | |
| | CCTTCCCCTGCTATGATCCCTAAAGATTGGC | | EKTLITHSSQPARFLGYDIRVRRSGTIKRS | |
| | CTCTGATCATCATCGACCTGAAAGACTGCTT | | GKVKKRTINGSVELLIPLQDKIRQFIFDKK | |
| | CTTCACAATCCCACTCGCCGAGCAGGATTGC | | IAIQKKDSSWFPVHRKYLIRSTDLEIITIY | |
| | GAGAAGTTCGCCTTCACCATCCCCGCCATCA | | NSELRGICNYYGLASNENQLNYFAYLMEYS | |
| | ACAACAAGGAGCCTGCCACCAGATTCCAGTG | | CLKTIASKHKGTLSKTISMFKDGSGSWGIP | |
| | GAAGGTGCTGCCTCAGGGCATGCTGAATTCT | | YEIKQGKQRRYFANFSECKSPYQFTDEISQ | |
| | CCAACAATCTGCCAGACCTTCGTGGGCAGAG | | APVLYGYARNTLENRLKAKCCELCGTSDEN | |
| | CTCTGCAGCCTGTTAGAGAAAAATTCAGCGA | | TSYEIHHVNKVKNLKGKEKWEMAMIAKQRK | |
| | CTGCTACATCATTCACTACATCGATGACATC | | TLVVCFHCHRHVIHKHKSGGSKRTADGSEF | |
| | CTGTGCGCCGCTGAAACCAAGGATAAGTTGA | | EPKKKRKV* | |
| | TCGACTGTTACACCTTCCTGCAAGCCGAGGT | | | |
| | GGCCAATGCCGGACTGGCTATCGCCTCTGAT | | | |
| | AAGATCCAGACCAGCACACCTTTCCACTACC | | | |
| | TGGGCATGCAGATCGAGAACCGGAAGATCAA | | | |
| | GCCACAGAAAATCGAGATCAGAAAGGACACC | | | |
| | CTGAAGACCCTGAACGACTTCCAGAAACTCC | | | |
| | TGGGGGATATCAACTGGATCAGACCTACCCT | | | |
| | GGGAATCCCTACGTACGCCATGAGCAACCTG | | | |
| | TTCAGCATCCTGAGGGGCGACAGCGACCTGA | | | |
| | ACAGCAAGAGAATGCTGACCCCTGAGGCCAC | | | |
| | AAAAGAGATCAAGCTGGTGGAAGAGAAGATC | | | |
| | CAGTCTGCTCAAATCAACAGAATCGATCCCC | | | |
| | TGGCCCCTCTTCAGTTGCTGATTTTCGCCAC | | | |
| | TGCCCATAGCCCCACCGGCATTATCATCCAG | | | |
| | AACACCGACCTGGTGGAATGGTCTTTTCTGC | | | |
| | CCCACAGCACCGTGAAGACATTTACACTGTA | | | |
| | CCTGGACCAGATCGCCACCCTGATCGGCCAA | | | |
| | ACAAGACTGCGGATCATCAAGCTGTGTGGCA | | | |
| | ACGACCCCGACAAGATCGTGGTGCCTCTGAC | | | |
| | CAAGGAACAGGTGCGGCAGGCTTTTATTAAC | | | |
| | TCCGGCGCCTGGCAGATCGGACTGGCCAACT | | | |
| | TCGTTGGCATCATCGACAATCACTATCCTAA | | | |
| | GACCAAGATCTTCCAATTTCTGAAGCTGACC | | | |
| | ACCTGGATTCTGCCTAAGATTACAAGACGGG | | | |
| | AACCCCTGGAGAACGCCCTGACCGTGTTCAC | | | |
| | CGACGGATCTTCCAACGGCAAAGCCGCCTAC | | | |
| | ACCGGCCCTAAGGAAAGAGTGATTAAGACAC | | | |
| | CATACCAGAGCGCCCAGAGAGCCGAACTGGT | | | |
| | CGCCGTGATCACCGTGCTGCAGGACTTCGAC | | | |
| | CAGCCTATCAATATCATCAGCGACAGTGCCT | | | |
| | ATGTGGTGCAGGCCACCCGGGACGTGGAAAC | | | |
| | CGCCCTGATCAAGTACAGCATGGACGATCAG | | | |
| | CTCAACCAGCTGTTTAACCTGCTGCAGCAGA | | | |
| | CCGTGCGGAAGAGAAACTTCCCCTTCTACAT | | | |
| | CACCCACATCCGCGCCCACACCAACCTGCCC | | | |
| | GGCCCTCTGACAAAGGCCAATGAGCAGGCTG | | | |
| | ATCTGCTGGTGTCTAGCGCCCTGATTAAGGC | | | |
| | CCAGGAGCTGCACGCCTCTGGCGGCTCAAAA | | | |
| | AGAACCGCCGACGGCAGCGAATTCGAGCCCA | | | |
| | AGAAGAAGAGGAAAGTCTAA | | | |
| bpNLS | ATGAAACGGACAGCCGACGGAAGCGAGTTCG | 70 | MKRTADGSEFESPKKKRKVKPTMAILERIS | 71 |
| -LtrA | AGTCACCAAAGAAGAAGCGGAAAGTCAAGCC | | KNSQENIDEVFTRLYRYLLRPDIYYVAYQN | |
| RT-4 AA | CACAATGGCCATCCTGGAAAGAATCTCTAAG | | LYSNKGASTKGILDDTADGFSEEKIKKIIQ | |
| | AACAGCCAGGAGAACATCGACGAGGTGTTCA | | SLKDGTYYPQPVRRMYIAKKNSKKMRPLGI | |
| | CCAGGCTGTACCGGTACCTGCTGAGACCTGA | | PTFTDKLIQEAVRIILESIYEPVFEDVSHG | |
| | CATCTACTACGTGGCCTACCAGAACCTGTAC | | FRPQRSCHTALKTIKREFGGARWFVEGDIK | |
| | AGCAACAAAGGCGCTTCTACCAAGGGCATCC | | | |
| | TCGACGACACAGCCGACGGATTTAGCGAGGA | | | |
| | AAAAATCAAGAAGATCATCCAGAGCCTGAAG | | | |
| | GACGGCACCTACTATCCTCAACCTGTTAGAA | | | |
| | GAATGTATATCGCCAAGAAAAACAGCAAGAA | | | |
| | AATGCGGCCTCTCGGCATTCCAACATTCACA | | | |
| | GATAAACTGATCCAGGAGGCCGTGCGGATCA | | | |
| | TCCTGGAGTCCATCTACGAGCCTGTGTTCGA | | | |
| | GGACGTGAGCCACGGCTTTAGACCTCAACGT | | | |
| | TCTTGTCACACCGCCCTGAAAACCATCAAGA | | | |
| | GAGAGTTCGGCGGAGCTCGGTGGTTCGTGGA | | | |
| | AGGCGACATCAAGGGTTGTTTTGACAACATC | | | |
| | GACCACGTGACACTGATCGGCCTGATCAACC | | | |
| | TGAAGATTAAGGATATGAAGATGAGCCAACT | | | |
| | GATCTACAAGTTTCTGAAGGCCGGCTACCTG | | | |
| | GAAAACTGGCAGTATCACAAAACGTACAGCG | | | |
| | GCACACCTCAGGGCGGCATCCTGAGCCCTCT | | | |
| | GCTGGCTAATATCTACCTGCACGAGCTGGAC | | | |
| | AAGTTCGTGCTGCAGCTGAAAATGAAATTCG | | | |
| | ATAGAGAAAGCCCCGAGAGAATCACCCCTGA | | | |
| | GTACAGAGAGCTCCACAACGAGATCAAGAGA | | | |
| | ATCAGCCACCGGCTTAAGAAGCTGGAAGGCG | | | |
| | AGGAAAAAGCCAAGGTGCTGCTGGAATACCA | | | |
| | GGAGAAGCGGAAGCGGCTGCCTACTCTGCCC | | | |
| | TGCACCAGCCAGACCAACAAGGTGCTGAAGT | | | |
| | ACGTGCGGTACGCTGATGACTTCATCATTTC | | | |
| | TGTGAAGGGCTCCAAAGAGGATTGCCAGTGG | | | |
| | ATCAAGGAACAGCTGAAATTGTTTATCCATA | | | |
| | ACAAGCTGAAGATGGAGCTGTCCGAAGAAAA | | | |
| | GACCCTGATCACACACAGCTCCCAGCCAGCC | | | |
| | AGATTCCTGGGCTACGACATCAGAGTGCGGA | | | |
| | GGAGCGGCACCATCAAGAGAAGCGGCAAGGT | | | |
| | GAAAAAACGCACCCTGAACGGCAGCGTCGAG | | | |
| | CTGCTGATACCCCTACAGGACAAGATCAGAC | | | |
| | AGTTCATCTTCGACAAGAAAATCGCCATCCA | | | |
| | AAAGAAGGACAGCAGCTGGTTCCCCGTCCAT | | | |
| | AGAAAGTACCTGATTAGAAGCACCGATCTGG | | | |
| | AAATCATCACAATCTACAACTCTGAGCTGAG | | | |
| | AGGAATCTGCAACTACTACGGCCTGGCTAGC | | | |
| | AACTTCAACCAGCTGAATTACTTCGCCTACC | | | |
| | TGATGGAATACTCCTGCCTGAAGACCATCGC | | | |
| | CAGCAAGCACAAGGGTACCCTGTCGAAGACC | | | |
| | ATCAGCATGTTCAAGGATGGATCTGGCTCTT | | | |
| | GGGGCATCCCCTACGAGATCAAGCAGGGAAA | | | |
| | GCAGAGAAGATACTTCGCCAATTTCAGCGAG | | | |
| | TGCAAGAGCCCTTATCAGTTTACCGACGAGA | | | |
| | TCAGCCAGGCCCCTGTGCTGTACGGATATGC | | | |
| | CCGGAACACCCTCGAGAATAGACTGAAAGCC | | | |
| | AAGTGCTGCGAGCTGTGTGGCACATCTGATG | | | |
| | AAAATACCAGCTACGAGATCCACCACGTGAA | | | |
| | CAAGGTGAAGAACCTGAAGGGCAAGGAAAAG | | | |
| | TGGGAGATGGCCATGATCGCCAAGCAGAGAA | | | |
| | AGACACTGGTGGTGTGCTTCCACTGTCACCG | | | |
| | CCACGTAATCCATAAGCACAAGTCTGGCGGC | | | |
| | TCAAAAAGAACCGCCGACGGCAGCGAATTCG | | | |
| | AGCCCAAGAAGAAGAGGAAAGTCTAA | | | |
| bpNLS-TeI4c | ATGAAACGGACAGCCGACGGAAGCGAGTTCG | 72 | MKRTADGSEFESPKKKRKVETRQMAVEQTT | 73 |
| RT-4 AA | AGTCACCAAAGAAGAAGCGGAAAGTCGAAAC | | GAVINQTETSWHSIDWAKANREVKRLQVRI | |
| linker- | AAGGCAGATGGCCGTGGAACAGACCACCGGC | | AKAVKEGRWGKVKALQWLLTHSPYGKALAV | |
| bpNLS | GCCGTCACCAACCAGACAGAGACAAGCTGGC | | KRVTDNSGSKTPGVDGITWSTQEQKAQAIK | |
| | ACTCTATCGACTGGGCCAAAGCCAACCGAGA | | SLRRRGYKPQPLRRVYIPKANGKQRPLGIP | |
| | GGTGAAAAGACTGCAGGTTAGAATCGCCAAG | | TMKDRAMQALYALALEPVAETTADRNSYGF | |
| | GCCGTGAAAGAGGGCAGATGGGGAAAAGTGA | | RRGRCIADAATQCHITLAKTDRAQYVLDAD | |
| | AGGCCCTCCAGTGGCTCCTGACCCACAGCTT | | IAGCFDNISHEWLLANIPLDKRILRKWLKS | |
| | CTACGGCAAGGCCCTGGCCGTGAAGCGGGTG | | GFVWKQQLFPIHAGTPQGGVISPMLANMTL | |
| | ACAGATAATAGCGGCTCTAAGACACCCGGCG | | DGMEELLNKFPRAHKVKLIRYADDFVVIGE | |
| | TGGACGGAATCACCTGGTCCACCCAGGAACA | | TKEVLYIAGAVIQAFLKERGLTLSKEKTKI | |
| | GAAAGCTCAGGCCATCAAGTCTCTGAGAAGA | | VHIEEGFDFLGWNIRKYDGKLLIKPAKKNV | |
| | CGGGGCTACAAGCCTCAGCCTCTGCGAAGAG | | KAFLKKIRDTLRELRTAPQEIVIDTLNPII | |
| | TGTACATCCCAAAGGCCAATGGCAAGCAAAG | | RGWTNYHKNQASKETFVGVDHLIWQKLWRW | |
| | ACCTCTGGGCATCCCTACCATGAAAGATAGA | | ARRRHPSKSVRWVKSKYFIQIGNRKWMFGI | |
| | GCCATGCAGGCCCTGTATGCCCTGGCCCTGG | | WTKDKNGDPWAKHLIKASEIRIQRRGKIKA | |
| | AACCTGTGGCCGAGACGACCGCCGATCGGAA | | DANPFLPEWAEYFEQRKKLKEAPAQYRRTR | |
| | CAGCTACGGCTTTAGAAGAGGAAGATGCATC | | RELWKKQGGICPVCGGEIEQDMLTEIHHIL | |
| | GCTGACGCAGCTACACAGTGCCACATCACAC | | PKHKGGTDDLDNLVLIHINCHKQVHNRDGQ | |
| | TGGCAAAGACCGATCGTGCTCAGTACGTGCT | | HSRFLLKEGLSGGSKRTADGSEFEPKKKRK | |
| | GGATGCCGATATCGCCGGATGTTTTGACAAT | | V* | |
| | ATTAGCCACGAGTGGCTGCTGGCTAACATCC | | | |
| | CCCTGGACAAGCGGATCCTGAGAAAGTGGCT | | | |
| | GAAGTCCGGCTTTGTGTGGAAGCAGCAGCTG | | | |
| | TTCCCCATCCACGCCGGCACACCTCAAGGCG | | | |
| | GGGTGATCAGCCCTATGCTGGCGAACATGAC | | | |
| | CCTGGACGGCATGGAAGAGCTGCTGAACAAG | | | |
| | TTCCCTAGAGCCCACAAGGTGAAACTGATCC | | | |
| | GGTACGCCGACGATTTCGTGGTGACCGGCGA | | | |
| | GACCAAGGAAGTGCTGTACATAGCCGGAGCC | | | |
| | GTGATCCAGGCTTTCCTGAAGGAAAGAGGCC | | | |
| | TGACCCTGAGCAAGGAAAAGACCAAGATTGT | | | |
| | CCATATCGAGGAAGGGTTCGACTTCCTGGGC | | | |
| | TGGAACATCCGGAAATACGACGGCAAGCTGC | | | |
| | TGATCAAACCAGCCAAGAAGAACGTGAAGGC | | | |
| | CTTTCTCAAGAAGATCCGGGACACCCTGAGA | | | |
| | GAGCTGAGAACAGCCCCTCAGGAGATCGTGA | | | |
| | TCGATACCCTTAATCCAATCATTAGAGGCTG | | | |
| | GACTAACTATCACAAGAACCAGGCCAGCAAG | | | |
| | GAGACATTCGTAGGCGTCGACCACCTGATCT | | | |
| | GGCAGAAGCTGTGGCGGTGGGCCAGACGGCG | | | |
| | GCACCCCAGCAAGAGCGTGCGGTGGGTGAAG | | | |
| | TCCAAGTACTTCATCCAAATCGGCAACCGGA | | | |
| | AGTGGATGTTCGGCATCTGGACCAAGGACAA | | | |
| | GAACGGCGACCCCTGGGCCAAACATCTGATC | | | |
| | AAGGCTTCTGAGATCAGAATCCAGAGACGCG | | | |
| | GCAAGATCAAGGCCGACGCCAACCCCTTCCT | | | |
| | GCCTGAGTGGGCTGAGTACTTCGAGCAGCGG | | | |
| | AAGAAGCTGAAGGAAGCCCCTGCCCAATACA | | | |
| | GAAGAACCAGACGGGAACTGTGGAAGAAACA | | | |
| | GGGCGGAATCTGCCCTGTGTGTGGCGGCGAG | | | |
| | ATTGAGCAGGACATGCTGACAGAGATCCACC | | | |
| | ACATCCTGCCTAAGCACAAGGGGGGCACCGA | | | |
| | CGACCTGGACAACCTGGTGCTGATCCACACC | | | |
| | AACTGCCACAAACAGGTGCACAACAGAGATG | | | |
| | GACAGCACAGCAGATTCCTGCTGAAGGAAGG | | | |
| | CCTGTCTGGCGGCTCAAAAAGAACCGCCGAC | | | |
| | GGCAGCGAATTCGAGCCCAAGAAGAAGAGGA | | | |
| | AAGTCTAA | | | |
| bpNLS-Ma- | ATGAAACGGACAGCCGACGGAAGCGAGTTCG | 74 | MKRTADGSEFESPKKKRKVDETKPYEISKD | 75 |
| int5 | AGTCACCAAAGAAGAAGCGGAAAGTCGATGA | | IVQEAFQRVKANKGAAGVDDENIAAFESDL | |
| RT-4 AA | GACAAAGCCCTACGAGATTTCTAAGGACATC | | INNLYKIWNRMSSGCYFPPSVKAIEIPKKS | |
| linker- | GTGCAGGAGGCCTTTCAGAGAGTGAAAGCCA | | GGTRILGIPTVLDRVAQMVTKIYLEPQLEP | |
| bpNLS | ACAAGGGCGCCGCCGGCGTGGACGATGAAAA | | LFHPDSYGYRPGKSAADALAATRKRCWRYN | |
| | CATCGCCGCTTTTGAGAGCGACCTGACCAAC | | WLLEFDIKGLFDNINHDLLMKQVSMHIDKP | |
| | AACCTGTACAAGATCTGGAACAGAATGAGCA | | WIILYIQRWLKAPFQMADGTVNERTKGTPQ | |
| | GCGGCTGCTACTTCCCACCTAGCGTGAAGGC | | GGVVSPLLANLFLHYAFDQWMDSHHRYNPF | |
| | CATCGAAATCCCTAAGAAATCTGGGGGCACC | | ERYADDSVIHCRSREEAERLWIELDKRLSE | |
| | AGAATCCTGGGAATCCCCACAGTGCTGGACA | | FGLELHPSKTRIVYCKDDDRQGDYPETKED | |
| | GAGTGGCCCAGATGGTGACCAAAATCTACCT | | FLGYTFRPRRSKNKYGKHFINFTPAVSNTA | |
| | GGAACCCCAGCTGGAACCTCTGTTCCACCCC | | KKSMQQEIHDWRMHLKPDKTLEDLSHMFNP | |
| | GACAGCTACGGCTATAGACCCGGCAAGTCCG | | ILRGWVNYYGLFYKSELYCVLKHMNRVLTR | |
| | CCGCCGATGCCCTGGCTGCTACACGGAAGCG | | WAQRKYKKLAGHKRRARYWLGKIARRDPKL | |
| | GTGCTGGCGGTACAATTGGCTGCTGGAATTC | | FVHWQMGIFPEAGSGGSKRTADGSEFEPKK | |
| | GATATCAAGGGCCTCTTTGACAACATCAATC | | KRKV* | |
| | ACGACCTGCTGATGAAACAGGTGAGCATGCA | | | |
| | TACCGACAAGCCTTGGATCATCCTGTACATC | | | |
| | CAGCGCTGGCTGAAGGCCCCTTTCCAAATGG | | | |
| | CCGACGGCACAGTGAATGAGCGGACCAAGGG | | | |
| | CACCCCTCAGGGCGGAGTGGTGTCCCCACTG | | | |
| | CTGGCTAATCTGTTCCTGCACTACGCCTTCG | | | |
| | ACCAGTGGATGGACAGCCACCACAGATACAA | | | |
| | CCCCTTCGAGCGGTATGCCGACGACAGCGTG | | | |
| | ATCCACTGCAGATCTAGAGAGGAAGCCGAGA | | | |
| | GACTGTGGATCGAGCTGGATAAGAGACTGAG | | | |
| | CGAGTTCGGCCTGGAACTGCACCCAAGCAAG | | | |
| | ACAAGAATCGTGTACTGTAAAGACGATGATA | | | |
| | GACAGGGAGATTACCCTGAGACAAAATTCGA | | | |
| | CTTCCTGGGCTACACCTTCCGGCCTAGACGG | | | |
| | AGCAAGAACAAGTACGGAAAACATTTCATCA | | | |
| | ACTTCACCCCTGCCGTCTCCAACACCGCCAA | | | |
| | GAAGAGCATGCAGCAGGAGATCCACGATTGG | | | |
| | CGGATGCACCTGAAGCCTGACAAGACCCTGG | | | |
| | AGGACCTGTCTCACATGTTCAACCCTATCCT | | | |
| | GAGAGGCTGGGTCAACTACTACGGCCTGTTC | | | |
| | TACAAGTCTGAGCTGTACTGCGTGCTTAAGC | | | |
| | ACATGAACAGAGTTCTGACCCGGTGGGCTCA | | | |
| | AAGAAAATATAAGAAGCTGGCCGGCCACAAG | | | |
| | CGGAGAGCCAGATACTGGCTGGGCAAGATCG | | | |
| | CCAGAAGGGACCCCAAGCTGTTTGTGCACTG | | | |
| | GCAGATGGGCATTTTCCCTGAAGCTGGATCT | | | |
| | GGCGGCTCAAAAAGAACCGCCGACGGCAGCG | | | |
| | AATTCGAGCCCAAGAAGAAGAGGAAAGTCTA | | | |
| | A | | | |
| bpNLS- | ATGAAACGGACAGCCGACGGAAGCGAGTTCG | 76 | MKRTADGSEFESPKKKRKVALLERILARDN | 77 |
| GsI-IIc | AGTCACCAAAGAAGAAGCGGAAAGTCGCCCT | | LITALKRVEANQGAPGIDGVSTDQLRDYIR | |
| RT-4 AA | GCTGGAGCGGATCCTGGCCAGAGACAATCTG | | AHWSTIHAQLLAGTYRPAPVRRVEIPKPGG | |
| linker- | ATCACCGCCCTGAAAAGGGTTGAGGCCAACC | | GTRQLGIPTVVDRLIQQAILQELTPIEDPD | |
| bpNES | AGGGCGCCCCTGGCATCGACGGCGTGTCTAC | | FSSSSFGERPGRNAHDAVRQAQGYIQEGYR | |
| | AGACCAGCTGAGAGATTACATCAGAGCTCAT | | YVVDMDLEKFFDRVNHDILMSRVARKVKDK | |
| | TGGAGCACCATCCACGCCCAACTCCTCGCTG | | RVLKLIRAYLQAGVMIEGVKVQTEEGTPQG | |
| | GCACCTACAGACCCGCCCCTGTGCGGAGAGT | | GPLSPLLANILLDDLDKELEKRGLKFCRYA | |
| | GGAAATCCCCAAGCCTGGAGGAGGCACCAGA | | DDCNIYVKSLRAGQRVKQSIQRFLEKTLKL | |
| | CAGCTGGGAATCCCTACAGTGGTGGATAGAC | | KVNEEKSAVDRPWKRAFLGFSFTPERKARI | |
| | TGATCCAGCAGGCCATCCTGCAGGAGCTTAC | | RLAPRSIQRLKQRIRQLINPNWSISMPERI | |
| | ACCAATCTTTGATCCTGACTTCAGCAGCAGC | | HRVNQYVMGWIGYFRLVETPSVLQTIEGWI | |
| | TCTTTCGGCTTCCGGCCTGGCAGAAACGCCC | | RRRLRLCQWLQWKRVRTRIRELRALGLKET | |
| | ACGACGCCGTTCGGCAGGCCCAGGGCTACAT | | AVMEIANTRKGAWRTTKTPQLHQALGKTYW | |
| | CCAAGAGGGCTACCGGTACGTGGTGGACATG | | TAQGLKSLTQRYFELRQGSGGSKRTADGSE | |
| | GACCTGGAGAAATTCTTCGACAGAGTGAACC | | FEPKKKRKV* | |
| | ACGATATCCTGATGTCCAGAGTCGCCAGAAA | | | |
| | GGTCAAGGACAAGCGTGTGCTGAAACTGATC | | | |
| | CGGGCCTACCTGCAAGCTGGAGTGATGATCG | | | |
| | AGGGCGTGAAAGTGCAGACAGAGGAAGGAAC | | | |
| | CCCTCAGGGCGGCCCTTTGTCTCCTCTGCTC | | | |
| | GCTAACATCCTGCTGGACGACCTGGATAAGG | | | |
| | AGCTGGAAAAGAGAGGCCTGAAGTTCTGCAG | | | |
| | ATACGCCGATGACTGTAATATCTACGTGAAG | | | |
| | TCCCTGCGGGCCGGCCAGAGAGTGAAGCAGA | | | |
| | GCATCCAGAGGTTCCTGGAAAAGACACTGAA | | | |
| | GCTGAAGGTGAACGAGGAAAAGAGCGCCGTG | | | |
| | GACAGACCCTGGAAGCGGGCCTTCCTGGGAT | | | |
| | TTAGCTTCACCCCCGAAAGAAAGGCCAGAAT | | | |
| | CCGCCTGGCTCCCAGAAGCATCCAGCGGCTG | | | |
| | AAACAGCGGATTCGGCAGCTGACTAACCCCA | | | |
| | ACTGGTCCATCAGCATGCCTGAGAGAATTCA | | | |
| | CAGAGTGAATCAGTACGTGATGGGCTGGATC | | | |
| | GGCTATTTTAGACTGGTGGAGACACCTAGCG | | | |
| | TGCTGCAGACCATCGAGGGTTGGATTAGACG | | | |
| | GAGACTGAGACTGTGCCAGTGGCTGCAGTGG | | | |
| | AAGCGCGTGCGAACAAGAATCAGAGAGCTGC | | | |
| | GGGCCCTGGGCCTGAAGGAAACCGCCGTGAT | | | |
| | GGAAATCGCCAACACCAGAAAGGGCGCCTGG | | | |
| | CGGACCACCAAGACCCCACAGCTGCACCAGG | | | |
| | CTCTGGGCAAGACCTACTGGACCGCTCAGGG | | | |
| | CCTGAAAAGCCTGACACAGAGATATTTCGAG | | | |
| | CTGAGACAAGGCTCTGGCGGCTCAAAAAGAA | | | |
| | CCGCCGACGGCAGCGAATTCGAGCCCAAGAA | | | |
| | GAAGAGGAAAGTCTAA | | | |
| bpNLS- | ATGAAACGGACAGCCGACGGAAGCGAGTTCG | 78 | MKRTADGSEFESPKKKRKVDTSNLMEQILS | 79 |
| Marathon | AGTCACCAAAGAAGAAGCGGAAAGTCGACAC | | SDNLNRAYLQVVRNKGAEGVDGMKYTELKE | |
| RT-4 AA | CAGCAATCTGATGGAACAGATCCTGAGCAGC | | HLAKNGETIKGQLRTRKYKPQPARRVEIPK | |
| linker- | GACAACCTGAACCGGGCCTACCTGCAGGTGG | | PDGGVRNLGVPTVIDRFIQQAIAQVLTPIY | |
| bpNLS | TGAGAAATAAAGGCGCTGAAGGCGTTGATGG | | EEQFHDHSYGFRPNRCAQQAILTALNIMND | |
| | CATGAAGTACACCGAGCTGAAGGAGCATCTG | | GNDWIVDIDLEKFFDTVNHDKLMTLIGRTI | |
| | GCCAAGAACGGCGAGACAATCAAGGGCCAGC | | KDGDVISIVRKYLVSGIMIDDEYEDSIVGT | |
| | TGAGAACCAGAAAGTATAAGCCTCAGCCAGC | | PQGGNLSPLLANIMLNELDKEMEKRGLNFV | |
| | TAGACGGGTGGAAATCCCCAAGCCCGATGGC | | RYADDCIIMVGSEMSANRVMRNISRFIEEK | |
| | GGAGTGCGGAACCTGGGAGTGCCAACAGTCA | | LGLKVNMTKSKVDRPSGLKYLGFGFYFDPR | |
| | CAGACCGGTTCATCCAGCAGGCTATCGCCCA | | AHQFKAKPHAKSVAKFKKRMKELTCRSWGV | |
| | AGTGCTGACCCCTATCTACGAGGAACAGTTT | | SNSYKVEKLNQLIRGWINYFKIGSMKTLCK | |
| | CACGACCACTCTTACGGCTTCCGGCCCAACA | | ELDSRIRYRLRMCIWKQWKTPQNQEKNLVK | |
| | GATGCGCCCAGCAAGCCATCCTGACAGCCCT | | LGIDRNTARRVAYTGKRIAYVQNKGAVNVA | |
| | GAACATCATGAACGATGGTAATGACTGGATC | | ISNKRLASFGLISMLDYYIEKCVTCSGGSK | |
| | GTGGACATCGACCTGGAAAAGTTTTTCGATA | | RTADGSEFEPKKKRKV* | |
| | CCGTGAATCACGATAAGCTGATGACGCTGAT | | | |
| | TGGCAGAACCATCAAGGACGGCGACGTGATC | | | |
| | TCTATTGTGCGCAAGTACCTCGTGTCCGGCA | | | |
| | TCATGATCGATGACGAGTACGAAGATAGCAT | | | |
| | CGTGGGAACACCTCAGGGCGGCAACCTGTCT | | | |
| | CCTCTGCTGGCCAACATCATGCTGAACGAGC | | | |
| | TGGATAAGGAGATGGAAAAAAGGGGCCTGAA | | | |
| | CTTCGTGCGGTACGCCGACGACTGCATCATC | | | |
| | ATGGTCGGCTCCGAGATGAGCGCCAACAGAG | | | |
| | TCATGCGGAACATCAGCAGATTCATCGAAGA | | | |
| | GAAGCTGGGCCTGAAAGTGAACATGACCAAG | | | |
| | TCCAAGGTGGACAGACCTAGCGGACTGAAGT | | | |
| | ACTTGGGCTTTGGCTTCTACTTCGACCCCAG | | | |
| | AGCCCACCAGTTCAAGGCCAAGCCTCACGCC | | | |
| | AAGAGCGTGGCTAAGTTCAAAAAGAGAATGA | | | |
| | AAGAGCTGACCTGTAGAAGCTGGGGCGTGTC | | | |
| | TAACAGCTACAAGGTGGAAAAACTGAATCAA | | | |
| | CTGATCAGAGGCTGGATCAACTACTTCAAGA | | | |
| | TCGGCAGCATGAAGACCCTGTGTAAAGAGCT | | | |
| | GGACAGCAGAATCAGGTACAGACTGCGGATG | | | |
| | TGCATCTGGAAGCAGTGGAAAACCCCTCAGA | | | |
| | ACCAGGAGAAAAACCTGGTCAAGCTTGGAAT | | | |
| | TGACAGAAATACCGCCAGAAGAGTGGCCTAT | | | |
| | ACAGGCAAGCGAATCGCCTACGTGTGCAACA | | | |
| | AGGGCGCCGTGAACGTGGCTATCAGCAACAA | | | |
| | GCGGCTGGCCAGCTTCGGCCTGATCTCTATG | | | |
| | CTGGACTACTACATCGAGAAGTGCGTGACCT | | | |
| | GCTCTGGCGGCTCAAAAAGAACCGCCGACGG | | | |
| | CAGCGAATTCGAGCCCAAGAAGAAGAGGAAA | | | |
| | GTCTAA | | | |
| bpNLS- | ATGAAACGGACAGCCGACGGAAGCGAGTTCG | 80 | MKRTADGSEFESPKKKRKVDESNLMEQILS | 81 |
| Marathon | AGTCACCAAAGAAGAAGCGGAAAGTCGACAC | | SRNLNRAYLQVVRRKGAEGVDGMKYTELKE | |
| (D14R- | CAGCAATCTGATGGAACAGATCCTGAGCAGC | | HLAKNGETIKGQLRTRKYKPQPARRVEIPK | |
| N26R-D74R- | CGGAACCTGAACCGGGCCTACCTGCAGGTGG | | PRGGVRNLGVPTVIDRFIQQAIAQVLTPIY | |
| N116K- | TGAGACGGAAAGGCGCTGAAGGCGTTGATGG | | EEQFHDHSYGFRPKRCAQQAILTALNIMND | |
| N197R) | CATGAAGTACACCGAGCTGAAGGAGCATCTG | | GNDWIVDIDLEKFFDTVNHDKLMTLIGRTI | |
| RT-4 AA | GCCAAGAACGGCGAGACAATCAAGGGCCAGC | | KDGDVISIVRKYLVSGIMIDDEYEDSIVGT | |
| linker- | TGAGAACCAGAAAGTATAAGCCTCAGCCAGC | | PQGGRLSPLLANIMLNELDKEMEKRGLNFV | |
| | TAGACGGGTGGAAATCCCCAAGCCCCGGGGC | | RYADDCIIMVGSEMSANRVMRNISRFIEEK | |
| | GGAGTGCGGAACCTGGGAGTGCCAACAGTCA | | LGLKVNMTKSKVDRPSGLKYLGFGFYFDPR | |
| | CAGACCGGTTCATCCAGCAGGCTATCGCCCA | | AHQFKAKPHAKSVAKFKKRMKELTCRSWGV | |
| | AGTGCTGACCCCTATCTACGAGGAACAGTTT | | SNSYKVEKLNQLIRGWINYFKIGSMKILCK | |
| | CACGACCACTCTTACGGCTTCCGGCCCAAGA | | ELDSRIRYRLRMCIWKQWKTPQNQEKNLVK | |
| | GATGCGCCCAGCAAGCCATCCTGACAGCCCT | | LGIDRNTARRVAYTGKRIAYVQNKGAVNVA | |
| | GAACATCATGAACGATGGTAATGACTGGATC | | ISNKRLASPGLISMLDYYIEKCVTCSGGSK | |
| | GTGGACATCGACCTGGAAAAGTTTTTCGATA | | RTADGSEFEPKKKRKV* | |
| | CCGTGAATCACGATAAGCTGATGACGCTGAT | | | |
| | TGGCAGAACCATCAAGGACGGCGACGTGATC | | | |
| | TCTATTGTGCGCAAGTACCTCGTGTCCGGCA | | | |
| | TCATGATCGATGACGAGTACGAAGATAGCAT | | | |
| | CGTGGGAACACCTCAGGGCGGCCGGCTGTCT | | | |
| | CCTCTGCTGGCCAACATCATGCTGAACGAGC | | | |
| | TGGATAAGGAGATGGAAAAAAGGGGCCTGAA | | | |
| | CTTCGTGCGGTACGCCGACGACTGCATCATC | | | |
| | ATGGTCGGCTCCGAGATGAGCGCCAACAGAG | | | |
| | TCATGCGGAACATCAGCAGATTCATCGAAGA | | | |
| | GAAGCTGGGCCTGAAAGTGAACATGACCAAG | | | |
| | TCCAAGGTGGACAGACCTAGCGGACTGAAGT | | | |
| | ACTTGGGCTTTGGCTTCTACTTCGACCCCAG | | | |
| | AGCCCACCAGTTCAAGGCCAAGCCTCACGCC | | | |
| | AAGAGCGTGGCTAAGTTCAAAAAGAGAATGA | | | |
| | AAGAGCTGACCTGTAGAAGCTGGGGCGTGTC | | | |
| | TAACAGCTACAAGGTGGAAAAACTGAATCAA | | | |
| | CTGATCAGAGGCTGGATCAACTACTTCAAGA | | | |
| | TCGGCAGCATGAAGACCCTGTGTAAAGAGCT | | | |
| | GGACAGCAGAATCAGGTACAGACTGCGGATG | | | |
| | TGCATCTGGAAGCAGTGGAAAACCCCTCAGA | | | |
| | ACCAGGAGAAAAACCTGGTCAAGCTTGGAAT | | | |
| | TGACAGAAATACCGCCAGAAGAGTGGCCTAT | | | |
| | ACAGGCAAGCGAATCGCCTACGTGTGCAACA | | | |
| | AGGGCGCCGTGAACGTGGCTATCAGCAACAA | | | |
| | GCGGCTGGCCAGCTTCGGCCTGATCTCTATG | | | |
| | CTGGACTACTACATCGAGAAGTGCGTGACCT | | | |
| | GCTCTGGCGGCTCAAAAAGAACCGCCGACGG | | | |
| | CAGCGAATTCGAGCCCAAGAAGAAGAGGAAA | | | |
| | GTCTAA | | | |
| bpNLS- | ATGAAACGGACAGCCGACGGAAGCGAGTTCG | 82 | MKRTADGSEFESPKKKRKVDKKYSIGLDIG | 83 |
| nCas9 | AGTCACCAAAGAAGAAGCGGAAAGTCGACAA | | TNSVGWAVITDEYKVPSKKFKVLGNTDRHS | |
| (H840A)- | GAAGTACAGCATCGGCCTGGACATCGGCACC | | IKKNLIGALLFDSGETAEATRLKRTARRRY | |
| XTEN-MMLV | AACTCTGTGGGCTGGGCCGTGATCACCGACG | | TRRKNRICYLQEIFSNEMAKVDDSFFHRLE | |
| RT-4 AA | AGTACAAGGTGCCCAGCAAGAAATTCAAGGT | | ESFLVEEDKKHERHPIFGNIVDEVAYHEKY | |
| linker- | GCTGGGCAACACCGACCGGCACAGCATCAAG | | PTIYHLRKKLVDSTDKADLRLIYLALAHMI | |
| bpNLS-P2A- | AAGAACCTGATCGGAGCCCTGCTGTTCGACA | | KERGHFLIEGDLNPDNSDVDKLFIQLVQTY | |
| eGFP | GCGGCGAAACAGCCGAGGCCACCCGGCTGAA | | NQLFEENPINASGVDAKAILSARLSKSRRL | |
| | GAGAACCGCCAGAAGAAGATACACCAGACGG | | ENLIAQLPGEKKNGLFGNLIALSLGLIPNF | |
| | AAGAACCGGATCTGCTATCTGCAAGAGATCT | | KSNFDLAEDAKLQLSKDTYDDDLQNLLAQI | |
| | TCAGCAACGAGATGGCCAAGGTGGACGACAG | | GDQYADLFLAAKNLSDAILLSDILRVNTEI | |
| | CTTCTTCCACAGACTGGAAGAGTCCTTCCTG | | TKAPLSASMIKRYDEHHQDLTLLKALVRQQ | |
| | GTGGAAGAGGATAAGAAGCACGAGCGGCACC | | LPEKYKEIFFDQSKNGYAGYIDGGASQEEF | |
| | CCATCTTCGGCAACATCGTGGACGAGGTGGC | | YKFIKPILEKMDGTEELLVKLNREDLLRKQ | |
| | CTACCACGAGAAGTACCCCACCATCTACCAC | | RTFDNGSIPHQIHLGELHAILRRQEDFYPF | |
| | CTGAGAAAGAAACTGGTGGACAGCACCGACA | | LKDNREKIEKILTFRIPYYVGPLARGNSRF | |
| | AGGCCGACCTGCGGCTGATCTATCTGGCCCT | | AWMTRKSEETITPWNFEEVVDKGASAQSFI | |
| | GGCCCACATGATCAAGTTCCGGGGCCACTTC | | ERMTNFDKNLPNEKVLPKHSLLYEYFTVYN | |
| | CTGATCGAGGGCGACCTGAACCCCGACAACA | | ELTKVKYVTEGMRKPAFLSGEQKKAIVDLL | |
| | GCGACGTGGACAAGCTGTTCATCCAGCTGGT | | FKTNRKVTVKQLKEDYFKKIECFDSVEISG | |
| | GCAGACCTACAACCAGCTGTTCGAGGAAAAC | | VEDRFNASLGTYHDLLKIIKDKDFLDNEEN | |
| | CCCATCAACGCCAGCGGCGTGGACGCCAAGG | | EDILEDIVLTLTLFEDREMIEERLKTYAHL | |
| | CCATCCTGTCTGCCAGACTGAGCAAGAGCAG | | FDDKVMKQLKRRRYTGWGRLSRKLINGIRD | |
| | ACGGCTGGAAAATCTGATCGCCCAGCTGCCC | | KQSGKTILDELKSDGFANRNFMQLIHDDSL | |
| | GGCGAGAAGAAGAATGGCCTGTTCGGAAACC | | TEKEDIQKAQVSGQGDSLHEHIANLAGSPA | |
| | TGATTGCCCTGAGCCTGGGCCTGACCCCCAA | | IKKGILQTVKVVDELVKVMGRHKPENIVIE | |
| | CTTCAAGAGCAACTTCGACCTGGCCGAGGAT | | MARENQTTQKGQKNSRERMKRIEEGIKELG | |
| | GCCAAACTGCAGCTGAGCAAGGACACCTACG | | SQILKEHPVENTQLQNEKLYLYYLQNGRDM | |
| | ACGACGACCTGGACAACCTGCTGGCCCAGAT | | YVDQELDINRLSDYDVDAIVPQSFLKDDSI | |
| | CGGCGACCAGTACGCCGACCTGTTTCTGGCC | | DNKVLTRSDKNRGKSDNVPSEEVVKKMKNY | |
| | GCCAAGAACCTGTCCGACGCCATCCTGCTGA | | WRQLLNAKLITQRKEDNITKAERGGLSELD | |
| | GCGACATCCTGAGAGTGAACACCGAGATCAC | | KAGFIKRQLVETRQITKHVAQILDSRMNTK | |
| | CAAGGCCCCCCTGAGCGCCTCTATGATCAAG | | YDENDKLIREVKVITLKSKLVSDFRKDFQF | |
| | AGATACGACGAGCACCACCAGGACCTGACCC | | YKVREINNYHHAHDAYLNAVVGTALIKKYP | |
| | TGCTGAAAGCTCTCGTGCGGCAGCAGCTGCC | | KLESEFVYGDYKVYDVRKMIAKSEQEIGKA | |
| | TGAGAAGTACAAAGAGATTTTCTTCGACCAG | | TAKYFFYSNIMNFFKTEITLANGEIRKRPL | |
| | AGCAAGAACGGCTACGCCGGCTACATTGACG | | IETNGETGEIVWDKGRDFATVRKVLSMPQV | |
| | GCGGAGCCAGCCAGGAAGAGTTCTACAAGTT | | NIVKKTEVQTGGFSKESILPKRNSDKLIAR | |
| | CATCAAGCCCATCCTGGAAAAGATGGACGGC | | KKDWDPKKYGGFDSPTVAYSVLVVAKVEKG | |
| | ACCGAGGAACTGCTCGTGAAGCTGAACAGAG | | KSKKLKSVKELLGITIMERSSFEKNPIDEL | |
| | AGGACCTGCTGCGGAAGCAGCGGACCTTCGA | | EAKGYKEVKKDLIIKLPKYSLFELENGRKR | |
| | CAACGGCAGCATCCCCCACCAGATCCACCTG | | MLASAGELQKGNELALPSKYVNFLYLASHY | |
| | GGAGAGCTGCACGCCATTCTGCGGCGGCAGG | | EKLKGSPEDNEQKQLFVEQHKHYLDEIIEQ | |
| | AAGATTTTTACCCATTCCTGAAGGACAACCG | | ISEFSKRVILADANLDKVLSAYNKHRDKPI | |
| | GGAAAAGATCGAGAAGATCCTGACCTTCCGC | | REQAENIIHLFTLTNLGAPAAFKYFDTTID | |
| | ATCCCCTACTACGTGGGCCCTCTGGCCAGGG | | RKRYTSTKEVLDATLIHQSITGLYETRIDL | |
| | GAAACAGCAGATTCGCCTGGATGACCAGAAA | | SQLGGDSGGSSGGSSGSETPGTSESATPES | |
| | GAGCGAGGAAACCATCACCCCCTGGAACTTC | | SGGSSGGSSTLNIEDEYRLHETSKEPDVSL | |
| | GAGGAAGTGGTGGACAAGGGCGCTTCCGCCC | | GSTWLSDFPQAWAETGGMGLAVRQAPLIIP | |
| | AGAGCTTCATCGAGCGGATGACCAACTTCGA | | LKATSTPVSIKQYPMSQEARLGIKPHIQRL | |
| | TAAGAACCTGCCCAACGAGAAGGTGCTGCCC | | LDQGILVPCQSPWNTPLLPVKKPGTNDYRP | |
| | AAGCACAGCCTGCTGTACGAGTACTTCACCG | | VQDLREVNKRVEDIHPTVPNPYNLLSGLPP | |
| | TGTATAACGAGCTGACCAAAGTGAAATACGT | | SHQWYTVLDLKDAFFCLRLHPTSQPLFAFE | |
| | GACCGAGGGAATGAGAAAGCCCGCCTTCCTG | | WRDPEMGISGQLTWTRLPQGFKNSPTLFNE | |
| | AGCGGCGAGCAGAAAAAGGCCATCGTGGACC | | ALHRDLADFRIQHPDLILLQYVDQLLLAAT | |
| | TGCTGTTCAAGACCAACCGGAAAGTGACCGT | | SELDCQQGTRALLQTLGNLGYRASAKKAçI | |
| | GAAGCAGCTGAAAGAGGACTACTTCAAGAAA | | CQKQVKYLGYLLKEGQRWLTEARKETVMGQ | |
| | ATCGAGTGCTTCGACTCCGTGGAAATCTCCG | | PTPKTPRQLREFLGKAGFCRLFIPGFAEMA | |
| | GCGTGGAAGATCGGTTCAACGCCTCCCTGGG | | APLYPLTKPGTLENWGPDQQKAYQEIKQAL | |
| | CACATACCACGATCTGCTGAAAATTATCAAG | | LTAPALGLPDLTKPFELFVDEKQGYAKGVL | |
| | GACAAGGACTTCCTGGACAATGAGGAAAACG | | TQKLGPWRRPVAYLSKKLDPVAAGWPPCLR | |
| | AGGACATTCTGGAAGATATCGTGCTGACCCT | | MVAAIAVLIKDAGKLTMGQPLVILAPHAVE | |
| | GACACTGTTTGAGGACAGAGAGATGATCGAG | | ALVKQPPDRWLSNARMTHYQALLLDTDRVQ | |
| | GAACGGCTGAAAACCTATGCCCACCTGTTCG | | PGPVVALNPATLLPLPEEGLQHNCLDILAE | |
| | ACGACAAAGTGATGAAGCAGCTGAAGCGGCG | | AHGTRPDLTDQPLPDADHTWYTDGSSLLQE | |
| | GAGATACACCGGCTGGGGCAGGCTGAGCCGG | | GQRKAGAAVTTETEVIWAKALPAGTSAQRA | |
| | AAGCTGATCAACGGCATCCGGGACAAGCAGT | | ELIALTQALKMAEGKKLNVYTDSRYAFATA | |
| | CCGGCAAGACAATCCTGGATTTCCTGAAGTC | | HIHGEIYRRRGWLTSEGKEIKNKDEILALL | |
| | CGACGGCTTCGCCAACAGAAACTTCATGCAG | | KALFLPKRLSIIHCPGHQKGHSAEARGNRM | |
| | CTGATCCACGACGACAGCCTGACCTTTAAAG | | ADQAARKAAITETPDTSTLLIENSSPSGGS | |
| | AGGACATCCAGAAAGCCCAGGTGTCCGGCCA | | KRTADGSEFEPKKKRKVGSGATNFSLLKQA | |
| | GGGCGATAGCCTGCACGAGCACATTGCCAAT | | GDVEENPGPMVSKGEELFTGVVPILVELDG | |
| | CTGGCCGGCAGCCCCGCCATTAAGAAGGGCA | | DVNGHKFSVSGEGEGDATYGKLTLKFICTT | |
| | TCCTGCAGACAGTGAAGGTGGTGGACGAGCT | | GKLPVPWPTLVTTLTYGVQCFSRYPDHMKQ | |
| | CGTGAAAGTGATGGGCCGGCACAAGCCCGAG | | HDFFKSAMPEGYVQERTIFFKDDGNYKTRA | |
| | AACATCGTGATCGAAATGGCCAGAGAGAACC | | EVKFEGDTLVNRIELKGIDFKEDGNILGHK | |
| | AGACCACCCAGAAGGGACAGAAGAACAGCCG | | LEYNYNSHNVYIMADKQKNGIKVNFKIRHN | |
| | CGAGAGAATGAAGCGGATCGAAGAGGGCATC | | IEDGSVQLADHYQQNTPIGDGPVLLPDNHY | |
| | AAAGAGCTGGGCAGCCAGATCCTGAAAGAAC | | LSTQSALSKDPNEKRDHMVLLEFVTAAGIT | |
| | ACCCCGTGGAAAACACCCAGCTGCAGAACGA | | LGMDELYK* | |
| | GAAGCTGTACCTGTACTACCTGCAGAATGGG | | | |
| | CGGGATATGTACGTGGACCAGGAACTGGACA | | | |
| | TCAACCGGCTGTCCGACTACGATGTGGACGC | | | |
| | TATCGTGCCTCAGAGCTTTCTGAAGGACGAC | | | |
| | TCCATCGACAACAAGGTGCTGACCAGAAGCG | | | |
| | ACAAGAACCGGGGCAAGAGCGACAACGTGCC | | | |
| | CTCCGAAGAGGTCGTGAAGAAGATGAAGAAC | | | |
| | TACTGGCGGCAGCTGCTGAACGCCAAGCTGA | | | |
| | TTACCCAGAGAAAGTTCGACAATCTGACCAA | | | |
| | GGCCGAGAGAGGCGGCCTGAGCGAACTGGAT | | | |
| | AAGGCCGGCTTCATCAAGAGACAGCTGGTGG | | | |
| | AAACCCGGCAGATCACAAAGCACGTGGCACA | | | |
| | GATCCTGGACTCCCGGATGAACACTAAGTAC | | | |
| | GACGAGAATGACAAGCTGATCCGGGAAGTGA | | | |
| | AAGTGATCACCCTGAAGTCCAAGCTGGTGTC | | | |
| | CGATTTCCGGAAGGATTTCCAGTTTTACAAA | | | |
| | GTGCGCGAGATCAACAACTACCACCACGCCC | | | |
| | ACGACGCCTACCTGAACGCCGTCGTGGGAAC | | | |
| | CGCCCTGATCAAAAAGTACCCTAAGCTGGAA | | | |
| | AGCGAGTTCGTGTACGGCGACTACAAGGTGT | | | |
| | ACGACGTGCGGAAGATGATCGCCAAGAGCGA | | | |
| | GCAGGAAATCGGCAAGGCTACCGCCAAGTAC | | | |
| | TTCTTCTACAGCAACATCATGAACTTTTTCA | | | |
| | AGACCGAGATTACCCTGGCCAACGGCGAGAT | | | |
| | CCGGAAGCGGCCTCTGATCGAGACAAACGGC | | | |
| | GAAACCGGGGAGATCGTGTGGGATAAGGGCC | | | |
| | GGGATTTTGCCACCGTGCGGAAAGTGCTGAG | | | |
| | CATGCCCCAAGTGAATATCGTGAAAAAGACC | | | |
| | GAGGTGCAGACAGGCGGCTTCAGCAAAGAGT | | | |
| | CTATCCTGCCCAAGAGGAACAGCGATAAGCT | | | |
| | GATCGCCAGAAAGAAGGACTGGGACCCTAAG | | | |
| | AAGTACGGCGGCTTCGACAGCCCCACCGTGG | | | |
| | CCTATTCTGTGCTGGTGGTGGCCAAAGTGGA | | | |
| | AAAGGGCAAGTCCAAGAAACTGAAGAGTGTG | | | |
| | AAAGAGCTGCTGGGGATCACCATCATGGAAA | | | |
| | GAAGCAGCTTCGAGAAGAATCCCATCGACTT | | | |
| | TCTGGAAGCCAAGGGCTACAAAGAAGTGAAA | | | |
| | AAGGACCTGATCATCAAGCTGCCTAAGTACT | | | |
| | CCCTGTTCGAGCTGGAAAACGGCCGGAAGAG | | | |
| | AATGCTGGCCTCTGCCGGCGAACTGCAGAAG | | | |
| | GGAAACGAACTGGCCCTGCCCTCCAAATATG | | | |
| | TGAACTTCCTGTACCTGGCCAGCCACTATGA | | | |
| | GAAGCTGAAGGGCTCCCCCGAGGATAATGAG | | | |
| | CAGAAACAGCTGTTTGTGGAACAGCACAAGC | | | |
| | ACTACCTGGACGAGATCATCGAGCAGATCAG | | | |
| | CGAGTTCTCCAAGAGAGTGATCCTGGCCGAC | | | |
| | GCTAATCTGGACAAAGTGCTGTCCGCCTACA | | | |
| | ACAAGCACCGGGATAAGCCCATCAGAGAGCA | | | |
| | GGCCGAGAATATCATCCACCTGTTTACCCTG | | | |
| | ACCAATCTGGGAGCCCCTGCCGCCTTCAAGT | | | |
| | ACTTTGACACCACCATCGACCGGAAGAGGTA | | | |
| | CACCAGCACCAAAGAGGTGCTGGACGCCACC | | | |
| | CTGATCCACCAGAGCATCACCGGCCTGTACG | | | |
| | AGACACGGATCGACCTGTCTCAGCTGGGAGG | | | |
| | TGACTCTGGAGGATCTAGCGGAGGATCCTCT | | | |
| | GGCAGCGAGACACCAGGAACAAGCGAGTCAG | | | |
| | CAACACCAGAGAGCAGTGGCGGCAGCAGCGG | | | |
| | CGGCAGCAGCACCCTAAATATAGAAGATGAG | | | |
| | TATCGGCTACATGAGACCTCAAAAGAGCCAG | | | |
| | ATGTTTCTCTAGGGTCCACATGGCTGTCTGA | | | |
| | TTTTCCTCAGGCCTGGGCGGAAACCGGGGGC | | | |
| | ATGGGACTGGCAGTTCGCCAAGCTCCTCTGA | | | |
| | TCATACCTCTGAAAGCAACCTCTACCCCCGT | | | |
| | GTCCATAAAACAATACCCCATGTCACAAGAA | | | |
| | GCCAGACTGGGGATCAAGCCCCACATACAGA | | | |
| | GACTGTTGGACCAGGGAATACTGGTACCCTG | | | |
| | CCAGTCCCCCTGGAACACGCCCCTGCTACCC | | | |
| | GTTAAGAAACCAGGGACTAATGATTATAGGC | | | |
| | CTGTCCAGGATCTGAGAGAAGTCAACAAGCG | | | |
| | GGTGGAAGACATCCACCCCACCGTGCCCAAC | | | |
| | CCTTACAACCTCTTGAGCGGGCTCCCACCGT | | | |
| | CCCACCAGTGGTACACTGTGCTTGATTTAAA | | | |
| | GGATGCCTTTTTCTGCCTGAGACTCCACCCC | | | |
| | ACCAGTCAGCCTCTCTTCGCCTTTGAGTGGA | | | |
| | GAGATCCAGAGATGGGAATCTCAGGACAATT | | | |
| | GACCTGGACCAGACTCCCACAGGGTTTCAAA | | | |
| | AACAGTCCCACCCTGTTTAATGAGGCACTGC | | | |
| | ACAGAGACCTAGCAGACTTCCGGATCCAGCA | | | |
| | CCCAGACTTGATCCTGCTACAGTACGTGGAT | | | |
| | GACTTACTGCTGGCCGCCACTTCTGAGCTAG | | | |
| | ACTGCCAACAAGGTACTCGGGCCCTGTTACA | | | |
| | AACCCTAGGGAACCTCGGGTATCGGGCCTCG | | | |
| | GCCAAGAAAGCCCAAATTTGCCAGAAACAGG | | | |
| | TCAAGTATCTGGGGTATCTTCTAAAAGAGGG | | | |
| | TCAGAGATGGCTGACTGAGGCCAGAAAAGAG | | | |
| | ACTGTGATGGGGCAGCCTACTCCGAAGACCC | | | |
| | CTCGACAACTAAGGGAGTTCCTAGGGAAGGC | | | |
| | AGGCTTCTGTCGCCTCTTCATCCCTGGGTTT | | | |
| | GCAGAAATGGCAGCCCCCCTGTACCCTCTCA | | | |
| | CCAAACCGGGGACTCTGTTTAATTGGGGCCC | | | |
| | AGACCAACAAAAGGCCTATCAAGAAATCAAG | | | |
| | CAAGCTCTTCTAACTGCCCCAGCCCTGGGGT | | | |
| | TGCCAGATTTGACTAAGCCCTTTGAACTCTT | | | |
| | TGTCGACGAGAAGCAGGGCTACGCCAAAGGT | | | |
| | GTCCTAACGCAAAAACTGGGACCTTGGCGTC | | | |
| | GGCCGGTGGCCTACCTGTCCAAAAAGCTAGA | | | |
| | CCCAGTAGCAGCTGGGTGGCCCCCTTGCCTA | | | |
| | CGGATGGTAGCAGCCATTGCCGTACTGACAA | | | |
| | AGGATGCAGGCAAGCTAACCATGGGACAGCC | | | |
| | ACTAGTCATTCTGGCCCCCCATGCAGTAGAG | | | |
| | GCACTAGTCAAACAACCCCCCGACCGCTGGC | | | |
| | TTTCCAACGCCCGGATGACTCACTATCAGGC | | | |
| | CTTGCTTTTGGACACGGACCGGGTCCAGTTC | | | |
| | GGACCGGTGGTAGCCCTGAACCCGGCTACGC | | | |
| | TGCTCCCACTGCCTGAGGAAGGGCTGCAACA | | | |
| | CAACTGCCTTGATATCCTGGCCGAAGCCCAC | | | |
| | GGAACCCGACCCGACCTAACGGACCAGCCGC | | | |
| | TCCCAGACGCCGACCACACCTGGTACACGGA | | | |
| | TGGAAGCAGTCTCTTACAAGAGGGACAGCGT | | | |
| | AAGGCGGGAGCTGCGGTGACCACCGAGACCG | | | |
| | AGGTAATCTGGGCTAAAGCCCTGCCAGCCGG | | | |
| | GACATCCGCTCAGCGGGCTGAACTGATAGCA | | | |
| | CTCACCCAGGCCCTAAAGATGGCAGAAGGTA | | | |
| | AGAAGCTAAATGTTTATACTGATAGCCGTTA | | | |
| | TGCTTTTGCTACTGCCCATATCCATGGAGAA | | | |
| | ATATACAGAAGGCGTGGGTGGCTCACATCAG | | | |
| | AAGGCAAAGAGATCAAAAATAAAGACGAGAT | | | |
| | CTTGGCCCTACTAAAAGCCCTCTTTCTGCCC | | | |
| | AAAAGACTTAGCATAATCCATTGTCCAGGAC | | | |
| | ATCAAAAGGGACACAGCGCCGAGGCTAGAGG | | | |
| | CAACCGGATGGCTGACCAAGCGGCCCGAAAG | | | |
| | GCAGCCATCACAGAGACTCCAGACACCTCTA | | | |
| | CCCTCCTCATAGAAAATTCATCACCCTCTGG | | | |
| | CGGCTCAAAAAGAACCGCCGACGGCAGCGAA | | | |
| | TTCGAGCCCAAGAAGAAGAGGAAAGTCGGAA | | | |
| | GCGGAGCTACTAACTTCAGCCTGCTGAAGCA | | | |
| | GGCTGGAGACGTGGAGGAGAACCCTGGACCT | | | |
| | ATGGTGAGCAAGGGCGAGGAGCTGTTCACCG | | | |
| | GGGTGGTGCCCATCCTGGTCGAGCTGGACGG | | | |
| | CGACGTAAACGGCCACAAGTTCAGCGTGTCC | | | |
| | GGCGAGGGCGAGGGCGATGCCACCTACGGCA | | | |
| | AGCTGACCCTGAAGTTCATCTGCACCACCGG | | | |
| | CAAGCTGCCCGTGCCCTGGCCCACCCTCGTG | | | |
| | ACCACCCTGACCTATGGAGTGCAGTGCTTCA | | | |
| | GCCGCTACCCCGACCACATGAAGCAGCACGA | | | |
| | CTTCTTCAAGTCCGCCATGCCCGAAGGCTAC | | | |
| | GTCCAGGAGCGCACCATCTTCTTCAAGGACG | | | |
| | ACGGCAACTACAAGACCCGCGCCGAGGTGAA | | | |
| | GTTCGAGGGCGACACCCTGGTGAACCGCATC | | | |
| | GAGCTGAAGGGCATCGACTTCAAGGAGGACG | | | |
| | GCAACATCCTGGGGCACAAGCTGGAGTACAA | | | |
| | CTACAACAGCCACAACGTCTATATCATGGCC | | | |
| | GACAAGCAGAAGAACGGCATCAAGGTGAACT | | | |
| | TCAAGATCCGCCACAACATCGAGGACGGCAG | | | |
| | CGTGCAGCTCGCCGACCACTACCAGCAGAAC | | | |
| | ACCCCCATCGGCGACGGCCCCGTGCTGCTGC | | | |
| | CCGACAACCACTACCTGAGCACCCAGTCCGC | | | |
| | CCTGAGCAAAGACCCCAACGAGAAGCGCGAT | | | |
| | CACATGGTCCTGCTGGAGTTCGTGACCGCCG | | | |
| | CCGGGATCACTCTCGGCATGGACGAGCTGTA | | | |
| | CAAGTAA | | | |
| bpNLS-MMLV | ATGAAACGGACAGCCGACGGAAGCGAGTTCG | 84 | MKRTADGSEFESPKKKRKVTLNIEDEYRLH | |
| RT- | AGTCACCAAAGAAGAAGCGGAAAGTCACCCT | | ETSKEPDVSLGSTWLSDFPQAWAETGGMGL | |
| XTEN- | AAATATAGAAGATGAGTATCGGCTACATGAG | | AVRQAPLIIPLKATSTPVSIKQYPMSQEAR | |
| nCas9 | ACCTCAAAAGAGCCAGATGTTTCTCTAGGGT | | LGIKPHIQRLLDQGILVPCQSPWNTPLLPV | |
| (H840A)- | CCACATGGCTGTCTGATTTTCCTCAGGCCTG | | KKPGTNDYRPVQDLREVNKRVEDIHPTVPN | |
| 4 AA | GGCGGAAACCGGGGGCATGGGACTGGCAGTT | | PYNLLSGLPPSHQWYTVLDLKDAFFCLRLH | |
| | CGCCAAGCTCCTCTGATCATACCTCTGAAAG | | PTSQPLFAFEWRDPEMGISGQLTWTRLPQG | |
| | CAACCTCTACCCCCGTGTCCATAAAACAATA | | FKNSPTLFNEALHRDLADFRIQHPDLILLQ | |
| | CCCCATGTCACAAGAAGCCAGACTGGGGATC | | YVDDLLLAATSELDCQQGTRALLQTLGNLG | |
| | AAGCCCCACATACAGAGACTGTTGGACCAGG | | YRASAKKAQICQKQVKYLGYLLKEGQRWLT | |
| | GAATACTGGTACCCTGCCAGTCCCCCTGGAA | | EARKETVMGQPTPKTPRQLREFLGKAGFCR | |
| | CACGCCCCTGCTACCCGTTAAGAAACCAGGG | | LFIPGFAEMAAPLYPLTKPGTLENWGPDQQ | |
| | ACTAATGATTATAGGCCTGTCCAGGATCTGA | | KAYQEIKQALLTAPALGLPDLTKPFELFVD | |
| | GAGAAGTCAACAAGCGGGTGGAAGACATCCA | | EKQGYAKGVLTQKLGPWRRPVAYLSKKLDP | |
| | CCCCACCGTGCCCAACCCTTACAACCTCTTG | | VAAGWPPCLRMVAAIAVLIKDAGKLIMGQP | |
| | AGCGGGCTCCCACCGTCCCACCAGTGGTACA | | LVILAPHAVEALVKQPPDRWLSNARMTHYQ | |
| | CTGTGCTTGATTTAAAGGATGCCTTTTTCTG | | ALLLDTDRVQFGPVVALNPATLLPLPEEGL | |
| | CCTGAGACTCCACCCCACCAGTCAGCCTCTC | | QHNCLDILAEAHGTRPDLTDQPLPDADHTW | |
| | TTCGCCTTTGAGTGGAGAGATCCAGAGATGG | | YTDGSSLLQEGQRKAGAAVTTETEVIWAKA | |
| | GAATCTCAGGACAATTGACCTGGACCAGACT | | LPAGTSAQRAELIALTQALKMAEGKKLNVY | |
| | CCCACAGGGTTTCAAAAACAGTCCCACCCTG | | TDSRYAFATAHIHGEIYRRRGWLTSEGKEI | |
| | TTTAATGAGGCACTGCACAGAGACCTAGCAG | | KNKDEILALLKALFLPKRLSIIHCPGHQKG | |
| | ACTTCCGGATCCAGCACCCAGACTTGATCCT | | HSAEARGNRMADQAARKAAITETPDTSTEL | |
| | GCTACAGTACGTGGATGACTTACTGCTGGCC | | IENSSPSGGSSGGSSGSETPGTSESATPES | |
| | GCCACTTCTGAGCTAGACTGCCAACAAGGT? | | SGGSSGGSSDKKYSIGLDIGTNSVGWAVIT | |
| | CTCGGGCCCTGTTACAAACCCTAGGGAACCT | | DEYKVPSKKFKVLGNTDRHSIKKNLIGALL | |
| | CGGGTATCGGGCCTCGGCCAAGAAAGCCCAA | | FDSGETAEATRLKRTARRRYTRRKNRICYL | |
| | ATTTGCCAGAAACAGGTCAAGTATCTGGGGT | | QEIFSNEMAKVDDSFFHRLEESFLVEEDKK | |
| | ATCTTCTAAAAGAGGGTCAGAGATGGCTGAC | | HERHPIFGNIVDEVAYHEKYPTIYHLRKKL | |
| | TGAGGCCAGAAAAGAGACTGTGATGGGGCAG | | VDSTDKADLRLIYLALAHMIKFRGHFLIEG | |
| | CCTACTCCGAAGACCCCTCGACAACTAAGGG | | DLNPDNSDVDKLFIQLVQTYNQLFEENPIN | |
| | AGTTCCTAGGGAAGGCAGGCTTCTGTCGCCT | | ASGVDAKAILSARLSKSRRLENLIAQLPGE | |
| | CTTCATCCCTGGGTTTGCAGAAATGGCAGCC | | KKNGLFGNLIALSLGLTPNFKSNFDLAEDA | |
| | CCCCTGTACCCTCTCACCAAACCGGGGACTC | | KLQLSKDTYDDDLDNLLAQIGDQYADLFLA | |
| | TGTTTAATTGGGGCCCAGACCAACAAAAGGC | | AKNLSDAILLSDILRVNTEITKAPLSASMI | |
| | CTATCAAGAAATCAAGCAAGCTCTTCTAACT | | KRYDEHHQDLTLLKALVRQQLPEKYKEIFF | |
| | GCCCCAGCCCTGGGGTTGCCAGATTTGACTA | | DQSKNGYAGYIDGGASQEEFYKFIKPILEK | |
| | AGCCCTTTGAACTCTTTGTCGACGAGAAGCA | | MDGTEELLVKLNREDLLRKQRTEDNGSIPH | |
| | GGGCTACGCCAAAGGTGTCCTAACGCAAAAA | | QIHLGELHAILRRQEDFYPFLKDNREKIEK | |
| | CTGGGACCTTGGCGTCGGCCGCTGGCCTACC | | ILTFRIPYYVGPLARGNSRFAWMTRKSEET | |
| | TGTCCAAAAAGCTAGACCCAGTAGCAGCTGG | | ITPWNFEEVVDKGASAQSFIERMTNEDKNL | |
| | GTGGCCCCCTTGCCTACGGATGGTAGCAGCC | | PNEKVLPKHSLLYEYFTVYNELTKVKYVTE | |
| | ATTGCCGTACTGACAAAGGATGCAGGCAAGC | | GMRKPAFLSGEQKKAIVDLLFKINRKVTVK | |
| | TAACCATGGGACAGCCACTAGTCATTCTGGC | | QLKEDYFKKIECFDSVEISGVEDRENASLG | |
| | CCCCCATGCAGTAGAGGCACTAGTCAAACAA | | TYHDLLKIIKDKDFLDNEENEDILEDIVLT | |
| | CCCCCCGACCGCTGGCTTTCCAACGCCCGGA | | LTLFEDREMIEERLKTYAHLFDDKVMKQLK | |
| | TGACTCACTATCAGGCCTTGCTTTTGGACAC | | RRRYTGWGRLSRKLINGIRDKQSGKTILDE | |
| | GGACCGGGTCCAGTTCGGACCGGTGGTAGCC | | LKSDGFANRNFMQLIHDDSLTFKEDIQKAQ | |
| | CTGAACCCGGCTACGCTGCTCCCACTGCCTG | | VSGQGDSLHEHIANLAGSPAIKKGILQTVK | |
| | AGGAAGGGCTGCAACACAACTGCCTTGATAT | | VVDELVKVMGRHKPENIVIEMARENQTTQK | |
| | CCTGGCCGAAGCCCACGGAACCCGACCCGAC | | GQKNSRERMKRIEEGIKELGSQILKEHPVE | |
| | CTAACGGACCAGCCGCTCCCAGACGCCGACC | | NTQLQNEKLYLYYLQNGRDMYVDQELDINR | |
| | ACACCTGGTACACGGATGGAAGCAGTCTCTT | | LSDYDVDAIVPQSFLKDDSIDNKVLTRSDK | |
| | ACAAGAGGGACAGCGTAAGGCCGGAGCTGCG | | NRGKSDNVPSEEVVKKMKNYWRQLLNAKLI | |
| | GTGACCACCGAGACCGAGGTAATCTGGGCTA | | TQRKFDNLTKAERGGLSELDKAGFIKRQLV | |
| | AAGCCCTGCCAGCCGGGACATCCGCTCAGCG | | ETRQITKHVAQILDSRMNTKYDENDKLIRE | |
| | GGCTGAACTGATAGCACTCACCCAGGCCCTA | | VKVITLKSKLVSDERKDFQFYKVREINNYH | |
| | AAGATGGCAGAAGGTAAGAAGCTAAATGTTT | | HAHDAYLNAVVGTALIKKYPKLESEFVYGD | |
| | ATACTGATAGCCGTTATGCTTTTGCTACTGC | | YKVYDVRKMIAKSEQEIGKATAKYFFYSNI | |
| | CCATATCCATGGAGAAATATACAGAAGGCGT | | MNFEKTEITLANGEIRKRPLIEINGETGEI | |
| | GGGTGGCTCACATCAGAAGGCAAAGAGATCA | | VWDKGRDFATVRKVLSMPQVNIVKKTEVQT | |
| | AAAATAAAGACGAGATCTTGGCCCTACTAAA | | GGFSKESILPKRNSDKLIARKKDWDPKKYG | |
| | AGCCCTCTTTCTGCCCAAAAGACTTAGCATA | | GFDSPTVAYSVLVVAKVEKGKSKKLKSVKE | |
| | ATCCATTGTCCAGGACATCAAAAGGGACACA | | LLGITIMERSSFEKNPIDFLEAKGYKEVKK | |
| | GCGCCGAGGCTAGAGGCAACCGGATGGCTGA | | DLIIKLPKYSLFELENGRKRMLASAGELQK | |
| | CCAAGCGGCCCGAAAGGCAGCCATCACAGAG | | GNELALPSKYVNFLYLASHYEKLKGSPEDN | |
| | ACTCCAGACACCTCTACCCTCCTCATAGAAA | | EQKQLFVEQHKHYLDEIIEQISEFSKRVIL | |
| | ATTCATCACCCTCTGGAGGATCTAGCGGAGG | | ADANLDKVLSAYNKHRDKPIREQAENIIHL | |
| | ATCCTCTGGCAGCGAGACACCAGGAACAAGC | | FTLTNLGAPAAFKYFDTTIDRKRYTSTKEV | |
| | GAGTCAGCAACACCAGAGAGCAGTGGCGGCA | | LDATLIHQSITGLYETRIDLSQLGGDSGGS | |
| | GCAGCGGCGGCAGCAGCGACAAGAAGTACAG | | KRTADGSEFEPKKKRKVGSGATNFSLLKQA | |
| | CATCGGCCTGGACATCGGCACCAACTCTGTG | | GDVEENPGPMVSKGEELFTGVVPILVELDG | |
| | GGCTGGGCCGTGATCACCGACGAGTACAAGG | | DVNGHKFSVSGEGEGDATYGKLTLKFICTT | |
| | TGCCCAGCAAGAAATTCAAGGTGCTGGGCAA | | GKLPVPWPTLVTTLTYGVQCFSRYPDHMKQ | |
| | CACCGACCGGCACAGCATCAAGAAGAACCTG | | HDFFKSAMPEGYVQERTIFFKDDGNYKTRA | |
| | ATCGGAGCCCTGCTGTTCGACAGCGGCGAAA | | EVKFEGDTLVNRIELKGIDFKEDGNILGHK | |
| | CAGCCGAGGCCACCCGGCTGAAGAGAACCGC | | LEYNYNSHNVYIMADKQKNGIKVNFKIRHN | |
| | CAGAAGAAGATACACCAGACGGAAGAACCGG | | IEDGSVQLADHYQQNTPIGDGPVLLPQNHY | |
| | ATCTGCTATCTGCAAGAGATCTTCAGCAACG | | LSTQSALSKDPNEKRDHMVLLEFVTAAGIT | |
| | AGATGGCCAAGGTGGACGACAGCTTCTTCCA | | LGMDELYK* | |
| | CAGACTGGAAGAGTCCTTCCTGGTGGAAGAG | | | |
| | GATAAGAAGCACGAGCGGCACCCCATCTTCG | | | |
| | GCAACATCGTGGACGAGGTGGCCTACCACGA | | | |
| | GAAGTACCCCACCATCTACCACCTGAGAAAG | | | |
| | AAACTGGTGGACAGCACCGACAAGGCCGACC | | | |
| | TGCGGCTGATCTATCTGGCCCTGGCCCACAT | | | |
| | GATCAAGTTCCGGGGCCACTTCCTGATCGAG | | | |
| | GGCGACCTGAACCCCGACAACAGCGACGTGG | | | |
| | ACAAGCTGTTCATCCAGCTGGTGCAGACCTA | | | |
| | CAACCAGCTGTTCGAGGAAAACCCCATCAAC | | | |
| | GCCAGCGGCGTGGACGCCAAGGCCATCCTGT | | | |
| | CTGCCAGACTGAGCAAGAGCAGACGGCTGGA | | | |
| | AAATCTGATCGCCCAGCTGCCCGGCGAGAAG | | | |
| | AAGAATGGCCTGTTCGGAAACCTGATTGCCC | | | |
| | TGAGCCTGGGCCTGACCCCCAACTTCAAGAG | | | |
| | CAACTTCGACCTGGCCGAGGATGCCAAACTG | | | |
| | CAGCTGAGCAAGGACACCTACGACGACGACC | | | |
| | TGGACAACCTGCTGGCCCAGATCGGCGACCA | | | |
| | GTACGCCGACCTGTTTCTGGCCGCCAAGAAC | | | |
| | CTGTCCGACGCCATCCTGCTGAGCGACATCC | | | |
| | TGAGAGTGAACACCGAGATCACCAAGGCCCC | | | |
| | CCTGAGCGCCTCTATGATCAAGAGATACGAC | | | |
| | GAGCACCACCAGGACCTGACCCTGCTGAAAG | | | |
| | CTCTCGTGCGGCAGCAGCTGCCTGAGAAGTA | | | |
| | CAAAGAGATTTTCTTCGACCAGAGCAAGAAC | | | |
| | GGCTACGCCGGCTACATTGACGGCGGAGCCA | | | |
| | GCCAGGAAGAGTTCTACAAGTTCATCAAGCC | | | |
| | CATCCTGGAAAAGATGGACGGCACCGAGGAA | | | |
| | CTGCTCGTGAAGCTGAACAGAGAGGACCTGC | | | |
| | TGCGGAAGCAGCGGACCTTCGACAACGGCAG | | | |
| | CATCCCCCACCAGATCCACCTGGGAGAGCTG | | | |
| | CACGCCATTCTGCGGCGGCAGGAAGATTTTT | | | |
| | ACCCATTCCTGAAGGACAACCGGGAAAAGAT | | | |
| | CGAGAAGATCCTGACCTTCCGCATCCCCTAC | | | |
| | TACGTGGGCCCTCTGGCCAGGGGAAACAGCA | | | |
| | GATTCGCCTGGATGACCAGAAAGAGCGAGGA | | | |
| | AACCATCACCCCCTGGAACTTCGAGGAAGTG | | | |
| | GTGGACAAGGGCGCTTCCGCCCAGAGCTTCA | | | |
| | TCGAGCGGATGACCAACTTCGATAAGAACCT | | | |
| | GCCCAACGAGAAGGTGCTGCCCAAGCACAGC | | | |
| | CTGCTGTACGAGTACTTCACCGTGTATAACG | | | |
| | AGCTGACCAAAGTGAAATACGTGACCGAGGG | | | |
| | AATGAGAAAGCCCGCCTTCCTGAGCGGCGAG | | | |
| | CAGAAAAAGGCCATCGTGGACCTGCTGTTCA | | | |
| | AGACCAACCGGAAAGTGACCGTGAAGCAGCT | | | |
| | GAAAGAGGACTACTTCAAGAAAATCGAGTGC | | | |
| | TTCGACTCCGTGGAAATCTCCGGCGTGGAAG | | | |
| | ATCGGTTCAACGCCTCCCTGGGCACATACCA | | | |
| | CGATCTGCTGAAAATTATCAAGGACAAGGAC | | | |
| | TTCCTGGACAATGAGGAAAACGAGGACATTC | | | |
| | TGGAAGATATCGTGCTGACCCTGACACTGTT | | | |
| | TGAGGACAGAGAGATGATCGAGGAACGGCTG | | | |
| | AAAACCTATGCCCACCTGTTCGACGACAAAG | | | |
| | TGATGAAGCAGCTGAAGCGGCGGAGATACAC | | | |
| | CGGCTGGGGCAGGCTGAGCCGGAAGCTGATC | | | |
| | AACGGCATCCGGGACAAGCAGTCCGGCAAGA | | | |
| | CAATCCTGGATTTCCTGAAGTCCGACGGCTT | | | |
| | CGCCAACAGAAACTTCATGCAGCTGATCCAC | | | |
| | GACGACAGCCTGACCTTTAAAGAGGACATCC | | | |
| | AGAAAGCCCAGGTGTCCGGCCAGGGCGATAG | | | |
| | CCTGCACGAGCACATTGCCAATCTGGCCGGC | | | |
| | AGCCCCGCCATTAAGAAGGGCATCCTGCAGA | | | |
| | CAGTGAAGGTGGTGGACGAGCTCGTGAAAGT | | | |
| | GATGGGCCGGCACAAGCCCGAGAACATCGTG | | | |
| | ATCGAAATGGCCAGAGAGAACCAGACCACCC | | | |
| | AGAAGGGACAGAAGAACAGCCGCGAGAGAAT | | | |
| | GAAGCGGATCGAAGAGGGCATCAAAGAGCTG | | | |
| | GGCAGCCAGATCCTGAAAGAACACCCCGTGG | | | |
| | AAAACACCCAGCTGCAGAACGAGAAGCTGTA | | | |
| | CCTGTACTACCTGCAGAATGGGCGGGATATG | | | |
| | TACGTGGACCAGGAACTGGACATCAACCGGC | | | |
| | TGTCCGACTACGATGTGGACGCTATCGTGCC | | | |
| | TCAGAGCTTTCTGAAGGACGACTCCATCGAC | | | |
| | AACAAGGTGCTGACCAGAAGCGACAAGAACC | | | |
| | GGGGCAAGAGCGACAACGTGCCCTCCGAAGA | | | |
| | GGTCGTGAAGAAGATGAAGAACTACTGGCGG | | | |
| | CAGCTGCTGAACGCCAAGCTGATTACCCAGA | | | |
| | GAAAGTTCGACAATCTGACCAAGGCCGAGAG | | | |
| | AGGCGGCCTGAGCGAACTGGATAAGGCCGGC | | | |
| | TTCATCAAGAGACAGCTGGTGGAAACCCGGC | | | |
| | AGATCACAAAGCACGTGGCACAGATCCTGGA | | | |
| | CTCCCGGATGAACACTAAGTACGACGAGAAT | | | |
| | GACAAGCTGATCCGGGAAGTGAAAGTGATCA | | | |
| | CCCTGAAGTCCAAGCTGGTGTCCGATTTCCG | | | |
| | GAAGGATTTCCAGTTTTACAAAGTGCGCGAG | | | |
| | ATCAACAACTACCACCACGCCCACGACGCCT | | | |
| | ACCTGAACGCCGTCGTGGGAACCGCCCTGAT | | | |
| | CAAAAAGTACCCTAAGCTGGAAAGCGAGTTC | | | |
| | GTGTACGGCGACTACAAGGTGTACGACGTGC | | | |
| | GGAAGATGATCGCCAAGAGCGAGCAGGAAAT | | | |
| | CGGCAAGGCTACCGCCAAGTACTTCTTCTAC | | | |
| | AGCAACATCATGAACTTTTTCAAGACCGAGA | | | |
| | TTACCCTGGCCAACGGCGAGATCCGGAAGCG | | | |
| | GCCTCTGATCGAGACAAACGGCGAAACCGGG | | | |
| | GAGATCGTGTGGGATAAGGGCCGGGATTTTG | | | |
| | CCACCGTGCGGAAAGTGCTGAGCATGCCCCA | | | |
| | AGTGAATATCGTGAAAAAGACCGAGGTGCAG | | | |
| | ACAGGCGGCTTCAGCAAAGAGTCTATCCTGC | | | |
| | CCAAGAGGAACAGCGATAAGCTGATCGCCAG | | | |
| | AAAGAAGGACTGGGACCCTAAGAAGTACGGC | | | |
| | GGCTTCGACAGCCCCACCGTGGCCTATTCTG | | | |
| | TGCTGGTGGTGGCCAAAGTGGAAAAGGGCAA | | | |
| | GTCCAAGAAACTGAAGAGTGTGAAAGAGCTG | | | |
| | CTGGGGATCACCATCATGGAAAGAAGCAGCT | | | |
| | TCGAGAAGAATCCCATCGACTTTCTGGAAGC | | | |
| | CAAGGGCTACAAAGAAGTGAAAAAGGACCTG | | | |
| | ATCATCAAGCTGCCTAAGTACTCCCTGTTCG | | | |
| | AGCTGGAAAACGGCCGGAAGAGAATGCTGGC | | | |
| | CTCTGCCGGCGAACTGCAGAAGGGAAACGAA | | | |
| | CTGGCCCTGCCCTCCAAATATGTGAACTTCC | | | |
| | TGTACCTGGCCAGCCACTATGAGAAGCTGAA | | | |
| | GGGCTCCCCCGAGGATAATGAGCAGAAACAG | | | |
| | CTGTTTGTGGAACAGCACAAGCACTACCTGG | | | |
| | ACGAGATCATCGAGCAGATCAGCGAGTTCTC | | | |
| | CAAGAGAGTGATCCTGGCCGACGCTAATCTG | | | |
| | GACAAAGTGCTGTCCGCCTACAACAAGCACC | | | |
| | GGGATAAGCCCATCAGAGAGCAGGCCGAGAA | | | |
| | TATCATCCACCTGTTTACCCTGACCAATCTG | | | |
| | GGAGCCCCTGCCGCCTTCAAGTACTTTGACA | | | |
| | CCACCATCGACCGGAAGAGGTACACCAGCAC | | | |
| | CAAAGAGGTGCTGGACGCCACCCTGATCCAC | | | |
| | CAGAGCATCACCGGCCTGTACGAGACACGGA | | | |
| | TCGACCTGTCTCAGCTGGGAGGTGACTCTGG | | | |
| | CGGCTCAAAAAGAACCGCCGACGGCAGCGAA | | | |
| | TTCGAGCCCAAGAAGAAGAGGAAAGTCGGAA | | | |
| | GCGGAGCTACTAACTTCAGCCTGCTGAAGCA | | | |
| | GGCTGGAGACGTGGAGGAGAACCCTGGACCT | | | |
| | ATGGTGAGCAAGGGCGAGGAGCTGTTCACCG | | | |
| | GGGTGGTGCCCATCCTGGTCGAGCTGGACGG | | | |
| | CGACGTAAACGGCCACAAGTTCAGCGTGTCC | | | |
| | GGCGAGGGCGAGGGCGATGCCACCTACGGCA | | | |
| | AGCTGACCCTGAAGTTCATCTGCACCACCGG | | | |
| | CAAGCTGCCCGTGCCCTGGCCCACCCTCGTG | | | |
| | ACCACCCTGACCTATGGAGTGCAGTGCTTCA | | | |
| | GCCGCTACCCCGACCACATGAAGCAGCACGA | | | |
| | CTTCTTCAAGTCCGCCATGCCCGAAGGCTAC | | | |
| | GTCCAGGAGCGCACCATCTTCTTCAAGGACG | | | |
| | ACGGCAACTACAAGACCCGCGCCGAGGTGAA | | | |
| | GTTCGAGGGCGACACCCTGGTGAACCGCATC | | | |
| | GAGCTGAAGGGCATCGACTTCAAGGAGGACG | | | |
| | GCAACATCCTGGGGCACAAGCTGGAGTACAA | | | |
| | CTACAACAGCCACAACGTCTATATCATGGCC | | | |
| | GACAAGCAGAAGAACGGCATCAAGGTGAACT | | | |
| | TCAAGATCCGCCACAACATCGAGGACGGCAG | | | |
| | CGTGCAGCTCGCCGACCACTACCAGCAGAAC | | | |
| | ACCCCCATCGGCGACGGCCCCGTGCTGCTGC | | | |
| | CCGACAACCACTACCTGAGCACCCAGTCCGC | | | |
| | CCTGAGCAAAGACCCCAACGAGAAGCGCGAT | | | |
| | CACATGGTCCTGCTGGAGTTCGTGACCGCCG | | | |
| | CCGGGATCACTCTCGGCATGGACGAGCTGTA | | | |
| | CAAGTAA | | | |
| bpNLS-nCas9 | ATGAAACGGACAGCCGACGGAAGCGAGTTCG | 86 | MKRTADGSEFESPKKKRKVDKKYSIGLDIG | 87 |
| (H840A) | AGTCACCAAAGAAGAAGCGGAAAGTCGACAA | | TNSVGWAVITDEYKVPSKKFKVLGNTDRHS | |
| pt. 1-32 AA | GAAGTACAGCATCGGCCTGGACATCGGCACC | | IKKNLIGALLFDSGETAEATRLKRTARRRY | |
| linker- | AACTCTGTGGGCTGGGCCGTGATCACCGACG | | TRRKNRICYLQEIFSNEMAKVDDSFFHRLE | |
| MMLV | AGTACAAGGTGCCCAGCAAGAAATTCAAGGT | | ESFLVEEDKKHERHPIFGNIVDEVAYHEKY | |
| RT-32 AA | GCTGGGCAACACCGACCGGCACAGCATCAAG | | PTIYHLRKKLVDSTDKADLRLIYLALAHMI | |
| linker- | AAGAACCTGATCGGAGCCCTGCTGTTCGACA | | KERGHFLIEGDLNPDNSDVDKLFIQLVQTY | |
| nCas9 | GCGGCGAAACAGCCGAGGCCACCCGGCTGAA | | NQLFEENPINASGVDAKAILSARLSKSRRL | |
| (H840A) | GAGAACCGCCAGAAGAAGATACACCAGACGG | | ENLIAQLPGEKKNGLFGNLIALSLGLIPNF | |
| pt. 2-4 AA | AAGAACCGGATCTGCTATCTGCAAGAGATCT | | KSNFDLAEDAKLQLSKDTYDDDLQNLLAQI | |
| linker- | TCAGCAACGAGATGGCCAAGGTGGACGACAG | | GDQYADLFLAAKNESDAILLSDILRVNTEI | |
| bpNLS-P2A- | CTTCTTCCACAGACTGGAAGAGTCCTTCCTG | | TKAPLSASMIKRYDEHHQDLTLLKALVRQQ | |
| eGFP | GTGGAAGAGGATAAGAAGCACGAGCGGCACC | | LPEKYKEIFFDQSKNGYAGYIDGGASQEEF | |
| --MMLV-RT | CCATCTTCGGCAACATCGTGGACGAGGTGGC | | YKFIKPILEKMDGTEELLVKLNREDLLRKQ | |
| inlaid at | CTACCACGAGAAGTACCCCACCATCTACCAC | | RTFDNGSIPHQIHLGELHAILRRQEDFYPF | |
| G1247 | CTGAGAAAGAAACTGGTGGACAGCACCGACA | | LKDNREKIEKILTFRIPYYVGPLARGNSRF | |
| | AGGCCGACCTGCGGCTGATCTATCTGGCCCT | | AWMTRKSEETITPWNFEEVVDKGASAQSFI | |
| | GGCCCACATGATCAAGTTCCGGGGCCACTTC | | ERMTNFDKNLPNEKVLPKHSLLYEYFTVYN | |
| | CTGATCGAGGGCGACCTGAACCCCGACAACA | | ELTKVKYVTEGMRKPAFLSGEQKKAIVDLL | |
| | GCGACGTGGACAAGCTGTTCATCCAGCTGGT | | FKTNRKVTVKQLKEDYFKKIECFDSVEISG | |
| | GCAGACCTACAACCAGCTGTTCGAGGAAAAC | | VEDRFNASLGTYHDLLKIIKDKDFLDNEEN | |
| | CCCATCAACGCCAGCGGCGTGGACGCCAAGG | | EDILEDIVETLTLFEDREMIEERLKTYAHL | |
| | CCATCCTGTCTGCCAGACTGAGCAAGAGCAG | | FDDKVMKQLKRRRYTGWGRLSRKLINGIRD | |
| | ACGGCTGGAAAATCTGATCGCCCAGCTGCCC | | KQSGKTILDELKSDGFANRNFMQLIHDDSL | |
| | GGCGAGAAGAAGAATGGCCTGTTCGGAAACC | | TFKEDIQKAQVSGQGDSLHEHIANLAGSPA | |
| | TGATTGCCCTGAGCCTGGGCCTGACCCCCAA | | IKKGILQTVKVVDELVKVMGRHKPENIVIE | |
| | CTTCAAGAGCAACTTCGACCTGGCCGAGGAT | | MARENQTTQKGQKNSRERMKRIEEGIKELG | |
| | GCCAAACTGCAGCTGAGCAAGGACACCTACG | | SQILKEHPVENTQLQNEKLYLYYLQNGRDM | |
| | ACGACGACCTGGACAACCTGCTGGCCCAGAT | | YVDQELDINRLSDYDVDAIVPQSFLKDDSI | |
| | CGGCGACCAGTACGCCGACCTGTTTCTGGCC | | DNKVLTRSDKNRGKSDNVPSEEVVKKMKNY | |
| | GCCAAGAACCTGTCCGACGCCATCCTGCTGA | | WRQLLNAKLITQRKEDNITKAERGGLSELD | |
| | GCGACATCCTGAGAGTGAACACCGAGATCAC | | KAGFIKRQLVETRQITKHVAQILDSRMNTK | |
| | CAAGGCCCCCCTGAGCGCCTCTATGATCAAG | | YDENDKLIREVKVITLKSKLVSDFRKDFQF | |
| | AGATACGACGAGCACCACCAGGACCTGACCC | | YKVREINNYHHAHDAYLNAVVGTALIKKYP | |
| | TGCTGAAAGCTCTCGTGCGGCAGCAGCTGCC | | KLESEFVYGDYKVYDVRKMIAKSEQEIGKA | |
| | TGAGAAGTACAAAGAGATTTTCTTCGACCAG | | TAKYFFYSNIMNFFKTEITLANGEIRKRPL | |
| | AGCAAGAACGGCTACGCCGGCTACATTGACG | | IETNGETGEIVWDKGRDFATVRKVLSMPQV | |
| | GCGGAGCCAGCCAGGAAGAGTTCTACAAGTT | | NIVKKTEVQTGGFSKESILPKRNSDKLIAR | |
| | CATCAAGCCCATCCTGGAAAAGATGGACGGC | | KKDWDPKKYGGFDSPTVAYSVLVVAKVEKG | |
| | ACCGAGGAACTGCTCGTGAAGCTGAACAGAG | | KSKKLKSVKELLGITIMERSSFEKNPIDFL | |
| | AGGACCTGCTGCGGAAGCAGCGGACCTTCGA | | EAKGYKEVKKDLIIKLPKYSLFELENGRKR | |
| | CAACGGCAGCATCCCCCACCAGATCCACCTG | | MLASAGELQKGNELALPSKYVNFLYLASHY | |
| | GGAGAGCTGCACGCCATTCTGCGGCGGCAGG | | EKLKGGGSSGGSSGSETPGTSESATPESSG | |
| | AAGATTTTTACCCATTCCTGAAGGACAACCG | | GSSGGSSTLNIEDEYRLHETSKEPDVSLGS | |
| | GGAAAAGATCGAGAAGATCCTGACCTTCCGC | | TWLSDFPQAWAETGGMGLAVRQAPLIIPLK | |
| | ATCCCCTACTACGTGGGCCCTCTGGCCAGGG | | ATSTPVSIKQYPMSQEARLGIKPHIQRLLD | |
| | GAAACAGCAGATTCGCCTGGATGACCAGAAA | | QGILVPCQSPWNTPLLPVKKPGTNDYRPVQ | |
| | GAGCGAGGAAACCATCACCCCCTGGAACTTC | | DLREVNKRVEDIHPTVPNPYNLLSGLPPSH | |
| | GAGGAAGTGGTGGACAAGGGCGCTTCCGCCC | | QWYTVLDLKDAFFCLRLHPTSQPLFAFEWR | |
| | AGAGCTTCATCGAGCGGATGACCAACTTCGA | | DPEMGISGQLTWIRLPQGFKNSPTLFNEAL | |
| | TAAGAACCTGCCCAACGAGAAGGTGCTGCCC | | HRDLADFRIQHPDLILLQYVDDLLLAATSE | |
| | AAGCACAGCCTGCTGTACGAGTACTTCACCG | | LDCQQGTRALLQTLGNLGYRASAKKAQICQ | |
| | GGAAACGAACTGGCCCTGCCCTCCAAATATG | | KQVKYLGYLLKEGQRWLTEARKETVMGQPT | |
| | TGAACTTCCTGTACCTGGCCAGCCACTATGA | | PKTPRQLREFLGKAGFCRLFIPGFAEMAAP | |
| | GAAGCTGAAGGGCGGAGGATCTAGCGGAGGA | | LYPLIKPGTLFNWGPDQQKAYQEIKQALLT | |
| | TCCTCTGGAAGCGAGACACCAGGCACAAGCG | | APALGLPDLTKPFELFVDEKQGYAKGVLTQ | |
| | AGTCCGCCACACCAGAGAGCTCCGGCGGCTC | | KLGPWRRPVAYLSKKLDPVAAGWPPCLRMV | |
| | CTCCGGAGGATCCTCTACCCTAAATATAGAA | | AAIAVLTKDAGKLTMGQPLVILAPHAVEAL | |
| | GATGAGTATCGGCTACATGAGACCTCAAAAG | | VKQPPDRWLSNARMTHYQALLLDTDRVQFG | |
| | AGCCAGATGTTTCTCTAGGGTCCACATGGCT | | PVVALNPATLLPLPEEGLQHNCLDILARAH | |
| | GTCTGATTTTCCTCAGGCCTGGGCGGAAACC | | GTRPDLIDQPLPDADHTWYTDGSSLLQEGQ | |
| | GGGGGCATGGGACTGGCAGTTCGCCAAGCTC | | RKAGAAVTTETEVIWAKALPAGTSAQRAEL | |
| | CTCTGATCATACCTCTGAAAGCAACCTCTAC | | IALTQALKMAEGKKLNVYTDSRYAFATAHI | |
| | CCCCGTGTCCATAAAACAATACCCCATGTCA | | HGEIYRRRGWLTSEGKEIKNKDEILALLKA | |
| | CAAGAAGCCAGACTGGGGATCAAGCCCCACA | | LFLPKRLSIIHCPGHQKGHSAEARGNRMAD | |
| | TACAGAGACTGTTGGACCAGGGAATACTGGT | | QAARKAAITETPDTSTLLIENSSPSGGSSG | |
| | ACCCTGCCAGTCCCCCTGGAACACGCCCCTG | | GSSGSETPGTSESATPESSGGSSGGSSPED | |
| | CTACCCGTTAAGAAACCAGGGACTAATGATT | | NEQKQLFVEQHKHYLDEIIEQISEFSKRVI | |
| | ATAGGCCTGTCCAGGATCTGAGAGAAGTCAA | | LADANLDKVLSAYNKHRDKPIREQAENIIH | |
| | CAAGCGGGTGGAAGACATCCACCCCACCGTG | | LFTLTNGAPAAFKYLEDIIEDRKRYTSTKE | |
| | CCCAACCCTTACAACCTCTTGAGCGGGCTCC | | VLDATLIHQSITGLYETRIDLSQLGGDSGG | |
| | CACCGTCCCACCAGTGGTACACTGTGCTTGA | | SKRTADGSEFEPKKKRKVGSGATNFSLLKQ | |
| | TTTAAAGGATGCCTTTTTCTGCCTGAGACTC | | AGDVEENPGPMVSKGEELFTGVVPILVELD | |
| | CACCCCACCAGTCAGCCTCTCTTCGCCTTTG | | GDVNGHKFSVSGEGEGDATYGKLTLKFICT | |
| | AGTGGAGAGATCCAGAGATGGGAATCTCAGG | | TGKLPVPWPTLVTTLTYGVQCFSRYPDHMK | |
| | ACAATTGACCTGGACCAGACTCCCACAGGGT | | QHDFFKSAMPEGYVQERTIFFKDDGNYKTR | |
| | TTCAAAAACAGTCCCACCCTGTTTAATGAGG | | AEVKFEGDTLVNRIELKGIDFKEDGNILGH | |
| | CACTGCACAGAGACCTAGCAGACTTCCGGAT | | KLEYNYNSHNVYIMADKQKNGIKVNFKIRH | |
| | CCAGCACCCAGACTTGATCCTGCTACAGTAC | | NIEDGSVQLADHYQQNTPIGDGPVLLPDNH | |
| | GTGGATGACTTACTGCTGGCCGCCACTTCTG | | YLSTQSALSKDPNEKRDHMVLLEFVTAAGI | |
| | AGCTAGACTGCCAACAAGGTACTCGGGCCCT | | TLGMDELYK* | |
| | GTTACAAACCCTAGGGAACCTCGGGTATCGG | | | |
| | GCCTCGGCCAAGAAAGCCCAAATTTGCCAGA | | | |
| | AACAGGTCAAGTATCTGGGGTATCTTCTAAA | | | |
| | AGAGGGTCAGAGATGGCTGACTGAGGCCAGA | | | |
| | AAAGAGACTGTGATGGGGCAGCCTACTCCGA | | | |
| | AGACCCCTCGACAACTAAGGGAGTTCCTAGG | | | |
| | GAAGGCAGGCTTCTGTCGCCTCTTCATCCCT | | | |
| | GGGTTTGCAGAAATGGCAGCCCCCCTGTACC | | | |
| | CTCTCACCAAACCGGGGACTCTGTTTAATTG | | | |
| | GGGCCCAGACCAACAAAAGGCCTATCAAGAA | | | |
| | ATCAAGCAAGCTCTTCTAACTGCCCCAGCCC | | | |
| | TGGGGTTGCCAGATTTGACTAAGCCCTTTGA | | | |
| | ACTCTTTGTCGACGAGAAGCAGGGCTACGCC | | | |
| | AAAGGTGTCCTAACGCAAAAACTGGGACCTT | | | |
| | GGCGTCGGCCGGTGGCCTACCTGTCCAAAAA | | | |
| | GCTAGACCCAGTAGCAGCTGGGTGGCCCCCT | | | |
| | TGCCTACGGATGGTAGCAGCCATTGCCGTAC | | | |
| | TGACAAAGGATGCAGGCAAGCTAACCATGGG | | | |
| | ACAGCCACTAGTCATTCTGGCCCCCCATGCA | | | |
| | GTAGAGGCACTAGTCAAACAACCCCCCGACC | | | |
| | GCTGGCTTTCCAACGCCCGGATGACTCACTA | | | |
| | TCAGGCCTTGCTTTTGGACACGGACCGGGTC | | | |
| | CAGTTCGGACCGGTGGTAGCCCTGAACCCGG | | | |
| | CTACGCTGCTCCCACTGCCTGAGGAAGGGCT | | | |
| | GCAACACAACTGCCTTGATATCCTGGCCGAA | | | |
| | GCCCACGGAACCCGACCCGACCTAACGGACC | | | |
| | AGCCGCTCCCAGACGCCGACCACACCTGGTA | | | |
| | CACGGATGGAAGCAGTCTCTTACAAGAGGGA | | | |
| | CAGCGTAAGGCGGGAGCTGCGGTGACCACCG | | | |
| | AGACCGAGGTAATCTGGGCTAAAGCCCTGCC | | | |
| | AGCCGGGACATCCGCTCAGCGGGCTGAACTG | | | |
| | ATAGCACTCACCCAGGCCCTAAAGATGGCAG | | | |
| | AAGGTAAGAAGCTAAATGTTTATACTGATAG | | | |
| | CCGTTATGCTTTTGCTACTGCCCATATCCAT | | | |
| | GGAGAAATATACAGAAGGCGTGGGTGGCTCA | | | |
| | CATCAGAAGGCAAAGAGATCAAAAATAAAGA | | | |
| | CGAGATCTTGGCCCTACTAAAAGCCCTCTTT | | | |
| | CTGCCCAAAAGACTTAGCATAATCCATTGTC | | | |
| | CAGGACATCAAAAGGGACACAGCGCCGAGGC | | | |
| | TAGAGGCAACCGGATGGCTGACCAAGCGGCC | | | |
| | CGAAAGGCAGCCATCACAGAGACTCCAGACA | | | |
| | CCTCTACCCTCCTCATAGAAAATTCATCACC | | | |
| | CTCCGGAGGATCTAGCGGAGGCTCCTCTGGC | | | |
| | TCTGAGACACCTGGCACAAGCGAGAGCGCAA | | | |
| | CACCTGAAAGCAGCGGGGGCAGCAGCGGGGG | | | |
| | GTCATCCCCCGAGGATAATGAGCAGAAACAG | | | |
| | CTGTTTGTGGAACAGCACAAGCACTACCTGG | | | |
| | ACGAGATCATCGAGCAGATCAGCGAGTTCTC | | | |
| | CAAGAGAGTGATCCTGGCCGACGCTAATCTG | | | |
| | GACAAAGTGCTGTCCGCCTACAACAAGCACC | | | |
| | GGGATAAGCCCATCAGAGAGCAGGCCGAGAA | | | |
| | TATCATCCACCTGTTTACCCTGACCAATCTG | | | |
| | GGAGCCCCTGCCGCCTTCAAGTACTTTGACA | | | |
| | CCACCATCGACCGGAAGAGGTACACCAGCAC | | | |
| | CAAAGAGGTGCTGGACGCCACCCTGATCCAC | | | |
| | CAGAGCATCACCGGCCTGTACGAGACACGGA | | | |
| | TCGACCTGTCTCAGCTGGGAGGTGACTCTGG | | | |
| | CGGCTCAAAAAGAACCGCCGACGGCAGCGAA | | | |
| | TTCGAGCCCAAGAAGAAGAGGAAAGTCGGAA | | | |
| | GCGGAGCTACTAACTTCAGCCTGCTGAAGCA | | | |
| | GGCTGGAGACGTGGAGGAGAACCCTGGACCT | | | |
| | ATGGTGAGCAAGGGCGAGGAGCTGTTCACCG | | | |
| | GGGTGGTGCCCATCCTGGTCGAGCTGGACGG | | | |
| | CGACGTAAACGGCCACAAGTTCAGCGTGTCC | | | |
| | GGCGAGGGCGAGGGCGATGCCACCTACGGCA | | | |
| | AGCTGACCCTGAAGTTCATCTGCACCACCGG | | | |
| | CAAGCTGCCCGTGCCCTGGCCCACCCTCGTG | | | |
| | ACCACCCTGACCTATGGAGTGCAGTGCTTCA | | | |
| | GCCGCTACCCCGACCACATGAAGCAGCACGA | | | |
| | CTTCTTCAAGTCCGCCATGCCCGAAGGCTAC | | | |
| | GTCCAGGAGCGCACCATCTTCTTCAAGGACG | | | |
| | ACGGCAACTACAAGACCCGCGCCGAGGTGAA | | | |
| | GTTCGAGGGCGACACCCTGGTGAACCGCATC | | | |
| | GAGCTGAAGGGCATCGACTTCAAGGAGGACG | | | |
| | GCAACATCCTGGGGCACAAGCTGGAGTACAA | | | |
| | CTACAACAGCCACAACGTCTATATCATGGCC | | | |
| | GACAAGCAGAAGAACGGCATCAAGGTGAACT | | | |
| | TCAAGATCCGCCACAACATCGAGGACGGCAG | | | |
| | CGTGCAGCTCGCCGACCACTACCAGCAGAAC | | | |
| | ACCCCCATCGGCGACGGCCCCGTGCTGCTGC | | | |
| | CCGACAACCACTACCTGAGCACCCAGTCCGC | | | |
| | CCTGAGCAAAGACCCCAACGAGAAGCGCGAT | | | |
| | CACATGGTCCTGCTGGAGTTCGTGACCGCCG | | | |
| | CCGGGATCACTCTCGGCATGGACGAGCTGTA | | | |
| | CAAGTAA | | | |
| bpNLS- | ATGAAACGGACAGCCGACGGAAGCGAGTTCG | 88 | MKRTADGSEFESPKKKRKVDKKYSIGLDIG | 89 |
| nCas9 | AGTCACCAAAGAAGAAGCGGAAAGTCGACAA | | TNSVGWAVITDEYKVPSKKFKVLGNTDRHS | |
| (H840A) | GAAGTACAGCATCGGCCTGGACATCGGCACC | | IKKNLIGALLFDSGETAEATRLKRTARRRY | |
| -XTEN- | AACTCTGTGGGCTGGGCCGTGATCACCGACG | | TRRKNRICYLQEIFSNEMAKVDDSFFHRLE | |
| 4 AA | AGTACAAGGTGCCCAGCAAGAAATTCAAGGT | | ESFLVEEDKKHERHPIFGNIVDEVAYHEKY | |
| linker- | GCTGGGCAACACCGACCGGCACAGCATCAAG | | PTIYHERKKLVDSQDKADLRLIYLALAHMI | |
| bpNLS- | AAGAACCTGATCGGAGCCCTGCTGTTCGACA | | KFRGHFLIEGDLNPDNSDVDKLFIQLVQTY | |
| P2A-eGFP | GCGGCGAAACAGCCGAGGCCACCCGGCTGAA | | NQLFEENPINASGVDAKAILSARLSKSRRL | |
| | GAGAACCGCCAGAAGAAGATACACCAGACGG | | ENLIAQLPGEKKNGLFGNLIALSLGLQPNF | |
| | AAGAACCGGATCTGCTATCTGCAAGAGATCT | | KSNFDLAEDAKLQLSKDTYDDDLDNLLAQI | |
| | TCAGCAACGAGATGGCCAAGGTGGACGACAG | | GDQYADLFLAAKNLSDAILLSDILRVNTEI | |
| | CTTCTTCCACAGACTGGAAGAGTCCTTCCTG | | TKAPLSASMIKRYDEHHQDLTLLKALVRQQ | |
| | GTGGAAGAGGATAAGAAGCACGAGCGGCACC | | LPEKYKEIFFDQSKNGYAGYIDGGASQEEF | |
| | CCATCTTCGGCAACATCGTGGACGAGGTGGC | | YKFIKPILEKMDGTEELLVKLNREDLLRKQ | |
| | CTACCACGAGAAGTACCCCACCATCTACCAC | | RTFDNGSIPHQIHLGELHAILRRQEDFYPF | |
| | CTGAGAAAGAAACTGGTGGACAGCACCGACA | | LKDNREKIEKILTFRIPYYVGPLARGNSRF | |
| | AGGCCGACCTGCGGCTGATCTATCTGGCCCT | | AWMTRKSEETITPWNFEEVVDKGASAQSFI | |
| | GGCCCACATGATCAAGTTCCGGGGCCACTTC | | ERMTNFDKNLPNEKVLPKHSLLYEYFTVYN | |
| | CTGATCGAGGGCGACCTGAACCCCGACAACA | | ELTKVKYVTEGMRKPAFLSGEQKKAIVDLL | |
| | GCGACGTGGACAAGCTGTTCATCCAGCTGGT | | FKTNRKVTVKQLKEDYFKKIECFDSVEISG | |
| | GCAGACCTACAACCAGCTGTTCGAGGAAAAC | | VEDRFNASLGTYHDLLKIIKDKDFLDNEEN | |
| | CCCATCAACGCCAGCGGCGTGGACGCCAAGG | | EDILEDIVLTLTLFEDREMIEERLKTYAHL | |
| | CCATCCTGTCTGCCAGACTGAGCAAGAGCAG | | FDDKVMKQLKRRRYTGWGRLSRKLINGIRD | |
| | ACGGCTGGAAAATCTGATCGCCCAGCTGCCC | | KQSGKTILDFLKSDGFANRNFMQLIHDDSL | |
| | GGCGAGAAGAAGAATGGCCTGTTCGGAAACC | | TFKEDIQKAQVSGQGDSLHEHIANLAGSPA | |
| | TGATTGCCCTGAGCCTGGGCCTGACCCCCAA | | IKKGILQTVKVVDELVKVMGRHKPENIVIE | |
| | CTTCAAGAGCAACTTCGACCTGGCCGAGGAT | | MARENQTTQKGQKNSRERMKRIEEGIKELG | |
| | GCCAAACTGCAGCTGAGCAAGGACACCTACG | | SQILKEHPVENTQLQNEKLYLYYLQNGRDM | |
| | ACGACGACCTGGACAACCTGCTGGCCCAGAT | | YVDQELDINRLSDYDVDAIVPQSFLKDDSI | |
| | CGGCGACCAGTACGCCGACCTGTTTCTGGCC | | DNKVLTRSDKNRGKSDNVPSEEVVKKMKNY | |
| | GCCAAGAACCTGTCCGACGCCATCCTGCTGA | | WRQLLNAKLITQRKFDNLTKAERGGLSELD | |
| | GCGACATCCTGAGAGTGAACACCGAGATCAC | | KAGFIKRQLVETRQITKHVAQILDSRMNTK | |
| | CAAGGCCCCCCTGAGCGCCTCTATGATCAAG | | YDENDKLIREVKVITLKSKLVSDFRKDFQF | |
| | AGATACGACGAGCACCACCAGGACCTGACCC | | YKVREINNYHHAHDAYLNAVVGTALIKKYP | |
| | TGCTGAAAGCTCTCGTGCGGCAGCAGCTGCC | | KLESEFVYGDYKVYDVRKMIAKSEQEIGKA | |
| | TGAGAAGTACAAAGAGAT″TTCTTCGACCAG | | TAKYFFYSNIMNFFKTEITLANGEIRKRPL | |
| | AGCAAGAACGGCTACGCCGGCTACATTGACG | | IETNGETGEIVWDKGRDFATVRKVLSMPQV | |
| | GCGGAGCCAGCCAGGAAGAGTTCTACAAGTT | | NIVKKTEVQTGGFSKESILPKRNSDKLIAR | |
| | CATCAAGCCCATCCTGGAAAAGATGGACGGC | | KKDWDPKKYGGFDSPTVAYSVLVVAKVEKG | |
| | ACCGAGGAACTGCTCGTGAAGCTGAACAGAG | | KSKKLKSVKELLGITIMERSSFEKNPIDEL | |
| | AGGACCTGCTGCGGAAGCAGCGGACCTTCGA | | BAKGYKEVKKDLIIKLPKYSLPELENGRKR | |
| | CAACGGCAGCATCCCCCACCAGATCCACCTG | | MLASAGELQKGNELALPSKYVNFLYLASHY | |
| | GGAGAGCTGCACGCCATTCTGCGGCGGCAGG | | EKLKGSPEDNEQKQLEVEQHKHYLDEIIBQ | |
| | AAGATTTTTACCCATTCCTGAAGGACAACCG | | ISEFSKRVILADANLDKVLSAYNKHRDKPI | |
| | GGAAAAGATCGAGAAGATCCTGACCTTCCGC | | REQAENIIHLFTLINLGAPAAFKYFDTTID | |
| | ATCCCCTACTACGTGGGCCCTCTGGCCAGGG | | RKRYTSTKEVLDATLIHQSITGLYETRIDL | |
| | GAAACAGCAGATTCGCCTGGATGACCAGAAA | | SQLGGDSGGSSGGSSGSETPGTSESATPES | |
| | GAGCGAGGAAACCATCACCCCCTGGAACTTC | | SGGSSGGSSSGGSKRTADGSEPEPKKKRKV | |
| | GAGGAAGTGGTGGACAAGGGCGCTTCCGCCC | | GSGATNFSLLKQAGDVEENPGPMVSKGEEL | |
| | AGAGCTTCATCGAGCGGATGACCAACTTCGA | | FTGVVPILVELDGDVNGHKFSVSGEGEGDA | |
| | TAAGAACCTGCCCAACGAGAAGGTGCTGCCC | | TYGKLTLKPICTTGKLPVPWPTLVTTLTYG | |
| | AAGCACAGCCTGCTGTACGAGTACTTCACCG | | VQCFSRYPDHMKQHDFFKSAMPEGYVQERT | |
| | TGTATAACGAGCTGACCAAAGTGAAATACGT | | IFFKDDGNYKTRAEVKFEGDTLVNRIELKG | |
| | GACCGAGGGAATGAGAAAGCCCGCCTTCCTG | | IDFKEDGNILGHKLEYNYNSHNVYIMADKQ | |
| | AGCGGCGAGCAGAAAAAGGCCATCGTGGACC | | KNGIKVNPKIRHNIEDGSVQLADHYQQNTP | |
| | TGCTGTTCAAGACCAACCGGAAAGTGACCGT | | IGDGPVLLPDNHYLSTQSALSKDPNEKRDH | |
| | GAAGCAGCTGAAAGAGGACTACTTCAAGAAA | | MVLLEFVTAAGITLGMDELYK* | |
| | ATCGAGTGCTTCGACTCCGTGGAAATCTCCG | | | |
| | GCGTGGAAGATCGGTTCAACGCCTCCCTGGG | | | |
| | CACATACCACGATCTGCTGAAAATTATCAAG | | | |
| | GACAAGGACTTCCTGGACAATGAGGAAAACG | | | |
| | AGGACATTCTGGAAGATATCGTGCTGACCCT | | | |
| | GACACTGTTTGAGGACAGAGAGATGATCGAG | | | |
| | GAACGGCTGAAAACCTATGCCCACCTGTTCG | | | |
| | ACGACAAAGTGATGAAGCAGCTGAAGCGGCG | | | |
| | GAGATACACCGGCTGGGGCAGGCTGAGCCGG | | | |
| | AAGCTGATCAACGGCATCCGGGACAAGCAGT | | | |
| | CCGGCAAGACAATCCTGGATTTCCTGAAGTC | | | |
| | CGACGGCTTCGCCAACAGAAACTTCATGCAG | | | |
| | CTGATCCACGACGACAGCCTGACCTTTAAAG | | | |
| | AGGACATCCAGAAAGCCCAGGTGTCCGGCCA | | | |
| | GGGCGATAGCCTGCACGAGCACATTGCCAAT | | | |
| | CTGGCCGGCAGCCCCGCCATTAAGAAGGGCA | | | |
| | TCCTGCAGACAGTGAAGGTGGTGGACGAGCT | | | |
| | CGTGAAAGTGATGGGCCGGCACAAGCCCGAG | | | |
| | AACATCGTGATCGAAATGGCCAGAGAGAACC | | | |
| | AGACCACCCAGAAGGGACAGAAGAACAGCCG | | | |
| | CGAGAGAATGAAGCGGATCGAAGAGGGCATC | | | |
| | AAAGAGCTGGGCAGCCAGATCCTGAAAGAAC | | | |
| | ACCCCGTGGAAAACACCCAGCTGCAGAACGA | | | |
| | GAAGCTGTACCTGTACTACCTGCAGAATGGG | | | |
| | CGGGATATGTACGTGGACCAGGAACTGGACA | | | |
| | TCAACCGGCTGTCCGACTACGATGTGGACGC | | | |
| | TATCGTGCCTCAGAGCTTTCTGAAGGACGAC | | | |
| | TCCATCGACAACAAGGTGCTGACCAGAAGCG | | | |
| | ACAAGAACCGGGGCAAGAGCGACAACGTGCC | | | |
| | CTCCGAAGAGGTCGTGAAGAAGATGAAGAAC | | | |
| | TACTGGCGGCAGCTGCTGAACGCCAAGCTGA | | | |
| | TTACCCAGAGAAAGTTCGACAATCTGACCAA | | | |
| | GGCCGAGAGAGGCGGCCTGAGCGAACTGGAT | | | |
| | AAGGCCGGCTTCATCAAGAGACAGCTGGTGG | | | |
| | AAACCCGGCAGATCACAAAGCACGTGGCACA | | | |
| | GATCCTGGACTCCCGGATGAACACTAAGTAC | | | |
| | GACGAGAATGACAAGCTGATCCGGGAAGTGA | | | |
| | AAGTGATCACCCTGAAGTCCAAGCTGGTGTC | | | |
| | CGATTTCCGGAAGGATTTCCAGTTTTACAAA | | | |
| | GTGCGCGAGATCAACAACTACCACCACGCCC | | | |
| | ACGACGCCTACCTGAACGCCGTCGTGGGAAC | | | |
| | CGCCCTGATCAAAAAGTACCCTAAGCTGGAA | | | |
| | AGCGAGTTCGTGTACGGCGACTACAAGGTGT | | | |
| | ACGACGTGCGGAAGATGATCGCCAAGAGCGA | | | |
| | GCAGGAAATCGGCAAGGCTACCGCCAAGTAC | | | |
| | TTCTTCTACAGCAACATCATGAACTTTTTCA | | | |
| | AGACCGAGATTACCCTGGCCAACGGCGAGAT | | | |
| | CCGGAAGCGGCCTCTGATCGAGACAAACGGC | | | |
| | GAAACCGGGGAGATCGTGTGGGATAAGGGCC | | | |
| | GGGATTTTGCCACCGTGCGGAAAGTGCTGAG | | | |
| | CATGCCCCAAGTGAATATCGTGAAAAAGACC | | | |
| | GAGGTGCAGACAGGCGGCTTCAGCAAAGAGT | | | |
| | CTATCCTGCCCAAGAGGAACAGCGATAAGCT | | | |
| | GATCGCCAGAAAGAAGGACTGGGACCCTAAG | | | |
| | AAGTACGGCGGCTTCGACAGCCCCACCGTGG | | | |
| | CCTATTCTGTGCTGGTGGTGGCCAAAGTGGA | | | |
| | AAAGGGCAAGTCCAAGAAACTGAAGAGTGTG | | | |
| | AAAGAGCTGCTGGGGATCACCATCATGGAAA | | | |
| | GAAGCAGCTTCGAGAAGAATCCCATCGACTT | | | |
| | TCTGGAAGCCAAGGGCTACAAAGAAGTGAAA | | | |
| | AAGGACCTGATCATCAAGCTGCCTAAGTACT | | | |
| | CCCTGTTCGAGCTGGAAAACGGCCGGAAGAG | | | |
| | AATGCTGGCCTCTGCCGGCGAACTGCAGAAG | | | |
| | GGAAACGAACTGGCCCTGCCCTCCAAATATG | | | |
| | TGAACTTCCTGTACCTGGCCAGCCACTATGA | | | |
| | GAAGCTGAAGGGCTCCCCCGAGGATAATGAG | | | |
| | CAGAAACAGCTGTTTGTGGAACAGCACAAGC | | | |
| | ACTACCTGGACGAGATCATCGAGCAGATCAG | | | |
| | CGAGTTCTCCAAGAGAGTGATCCTGGCCGAC | | | |
| | GCTAATCTGGACAAAGTGCTGTCCGCCTACA | | | |
| | ACAAGCACCGGGATAAGCCCATCAGAGAGCA | | | |
| | GGCCGAGAATATCATCCACCTGTTTACCCTG | | | |
| | ACCAATCTGGGAGCCCCTGCCGCCTTCAAGT | | | |
| | ACTTTGACACCACCATCGACCGGAAGAGGTA | | | |
| | CACCAGCACCAAAGAGGTGCTGGACGCCACC | | | |
| | CTGATCCACCAGAGCATCACCGGCCTGTACG | | | |
| | AGACACGGATCGACCTGTCTCAGCTGGGAGG | | | |
| | TGACTCTGGAGGATCTAGCGGAGGATCCTCT | | | |
| | GGCAGCGAGACACCAGGAACAAGCGAGTCAG | | | |
| | CAACACCAGAGAGCAGTGGCGGCAGCAGCGG | | | |
| | CGGCAGCAGCTCTGGCGGCTCAAAAAGAACC | | | |
| | GCCGACGGCAGCGAATTCGAGCCCAAGAAGA | | | |
| | AGAGGAAAGTCGGAAGCGGAGCTACTAACTT | | | |
| | CAGCCTGCTGAAGCAGGCTGGAGACGTGGAG | | | |
| | GAGAACCCTGGACCTATGGTGAGCAAGGGCG | | | |
| | AGGAGCTGTTCACCGGGGTGGTGCCCATCCT | | | |
| | GGTCGAGCTGGACGGCGACGTAAACGGCCAC | | | |
| | AAGTTCAGCGTGTCCGGCGAGGGCGAGGGCG | | | |
| | ATGCCACCTACGGCAAGCTGACCCTGAAGTT | | | |
| | CATCTGCACCACCGGCAAGCTGCCCGTGCCC | | | |
| | TGGCCCACCCTCGTGACCACCCTGACCTATG | | | |
| | GAGTGCAGTGCTTCAGCCGCTACCCCGACCA | | | |
| | CATGAAGCAGCACGACTTCTTCAAGTCCGCC | | | |
| | ATGCCCGAAGGCTACGTCCAGGAGCGCACCA | | | |
| | TCTTCTTCAAGGACGACGGCAACTACAAGAC | | | |
| | CCGCGCCGAGGTGAAGTTCGAGGGCGACACC | | | |
| | CTGGTGAACCGCATCGAGCTGAAGGGCATCG | | | |
| | ACTTCAAGGAGGACGGCAACATCCTGGGGCA | | | |
| | CAAGCTGGAGTACAACTACAACAGCCACAAC | | | |
| | GTCTATATCATGGCCGACAAGCAGAAGAACG | | | |
| | GCATCAAGGTGAACTTCAAGATCCGCCACAA | | | |
| | CATCGAGGACGGCAGCGTGCAGCTCGCCGAC | | | |
| | CACTACCAGCAGAACACCCCCATCGGCGACG | | | |
| | GCCCCGTGCTGCTGCCCGACAACCACTACCT | | | |
| | GAGCACCCAGTCCGCCCTGAGCAAAGACCCC | | | |
| | AACGAGAAGCGCGATCACATGGTCCTGCTGG | | | |
| | AGTTCGTGACCGCCGCCGGGATCACTCTCGG | | | |
| | CATGGACGAGCTGTACAAGTAA | | | |
| bpNLS-MMLV | ATGAAACGGACAGCCGACGGAAGCGAGTTCG | 90 | MKRTADGSEFESPKKKRKVILNIEDEYRLH | 91 |
| RT- | AGTCACCAAAGAAGAAGCGGAAAGTCACCCT | | ETSKEPDVSLGSTWLSDFPQAWAETGGMGL | |
| 4 AA | AAATATAGAAGATGAGTATCGGCTACATGAG | | AVRQAPLIIPLKATSTPVSIKQYPMSQEAR | |
| linker- | ACCTCAAAAGAGCCAGATGTTTCTCTAGGGT | | LGIKPHIQRLLDQGILVPCQSPWNTPLLPV | |
| bpNLS- | CCACATGGCTGTCTGATTTTCCTCAGGCCTG | | KKPGINDYRPVQDLREVNKRVEDIHPTVPN | |
| P2A-eGFP | GGCGGAAACCGGGGGCATGGGACTGGCAGTT | | PYNLLSGLPPSHQWYTVLDLKDAFFCLRLH | |
| | CGCCAAGCTCCTCTGATCATACCTCTGAAAG | | PTSQPLFAFEWRDPEMGISGQLTWIRLPQG | |
| | CAACCTCTACCCCCGTGTCCATAAAACAATA | | FKNSPTLFNEALHRDLADFRIQHPDLILLQ | |
| | CCCCATGTCACAAGAAGCCAGACTGGGGATC | | YVDDLLLAATSELDCQQGTRALLQTLGNLG | |
| | AAGCCCCACATACAGAGACTGTTGGACCAGG | | YRASAKKAQICQKQVKYLGYLLKEGQRWLT | |
| | GAATACTGGTACCCTGCCAGTCCCCCTGGAA | | EARKETVMGQPTPKTPRQLREFLGKAGFCR | |
| | CACGCCCCTGCTACCCGTTAAGAAACCAGGG | | LFIPGFAEMAAPLYPLIKPGTLENWGPDQQ | |
| | ACTAATGATTATAGGCCTGTCCAGGATCTGA | | KAYQEIKQALLTAPALGLPDLTKPFELFVD | |
| | GAGAAGTCAACAAGCGGGTGGAAGACATCCA | | EKQGYAKGVLTQKLGPWRRPVAYLSKKLDP | |
| | CCCCACCGTGCCCAACCCTTACAACCTCTTG | | VAAGWPPCLRMVAAIAVLTKDAGKLTMGQP | |
| | AGCGGGCTCCCACCGTCCCACCAGTGGTACA | | LVILAPHAVEALVKQPPDRWLSNARMTHYQ | |
| | CTGTGCTTGATTTAAAGGATGCCTTTTTCTG | | ALLLDTDRVQFGPVVALNPATLLPLPEEGL | |
| | CCTGAGACTCCACCCCACCAGTCAGCCTCTC | | QHNCLDILAEAHGTRPDLTDQPLPDADHTW | |
| | TTCGCCTTTGAGTGGAGAGATCCAGAGATGG | | YTDGSSLLQEGQRKAGAAVTTETEVIWAKA | |
| | GAATCTCAGGACAATTGACCTGGACCAGACT | | LPAGTSAQRAELIALTQALKMAEGKKLNVY | |
| | CCCACAGGGTTTCAAAAACAGTCCCACCCTG | | TDSRYAFATAHINGEIYRRRGWLTSEGKEI | |
| | TTTAATGAGGCACTGCACAGAGACCTAGCAG | | KNKDEILALLKALFLPKRLSIIHCPGHQKG | |
| | ACTTCCGGATCCAGCACCCAGACTTGATCCT | | HSAEARGNRMADQAARKAAITETPDTSTLL | |
| | GCTACAGTACGTGGATGACTTACTGCTGGCC | | IENSSPSGGSKRTADGSEFEPKKKRKVGSG | |
| | GCCACTTCTGAGCTAGACTGCCAACAAGGTA | | ATNFSLLKQAGDVEENPGPMVSKGEELFTG | |
| | CTCGGGCCCTGTTACAAACCCTAGGGAACCT | | VVPILVELDGDVNGHKESVSGEGEGDATYG | |
| | CGGGTATCGGGCCTCGGCCAAGAAAGCCCAA | | KLTLKFICTTGKLPVPWPTLVTTLTYGVQC | |
| | ATTTGCCAGAAACAGGTCAAGTATCTGGGGT | | FSRYPDHMKQHDFFKSAMPEGYVQERTIFF | |
| | ATCTTCTAAAAGAGGGTCAGAGATGGCTGAC | | KDDGNYKTRAEVKFEGDTLVNRIELKGIDE | |
| | TGAGGCCAGAAAAGAGACTGTGATGGGGCAG | | KEDGNILGHKLEYNYNSHNVYIMADKQKNG | |
| | CCTACTCCGAAGACCCCTCGACAACTAAGGG | | IKVNFKIRHNIEDGSVQLADHYQQNTPIGD | |
| | AGTTCCTAGGGAAGGCAGGCTTCTGTCGCCT | | GPVLLPDNHYLSTQSALSKDPNEKRDHMVL | |
| | CTTCATCCCTGGGTTTGCAGAAATGGCAGCC | | LEFVTAAGITLGMDELYK | |
| | CCCCTGTACCCTCTCACCAAACCGGGGACTC | | | |
| | TGTTTAATTGGGGCCCAGACCAACAAAAGGC | | | |
| | CTATCAAGAAATCAAGCAAGCTCTTCTAACT | | | |
| | GCCCCAGCCCTGGGGTTGCCAGATTTGACTA | | | |
| | AGCCCTTTGAACTCTTTGTCGACGAGAAGCA | | | |
| | GGGCTACGCCAAAGGTGTCCTAACGCAAAAA | | | |
| | CTGGGACCTTGGCGTCGGCCGGTGGCCTACC | | | |
| | TGTCCAAAAAGCTAGACCCAGTAGCAGCTGG | | | |
| | GTGGCCCCCTTGCCTACGGATGGTAGCAGCC | | | |
| | ATTGCCGTACTGACAAAGGATGCAGGCAAGC | | | |
| | TAACCATGGGACAGCCACTAGTCATTCTGGC | | | |
| | CCCCCATGCAGTAGAGGCACTAGTCAAACAA | | | |
| | CCCCCCGACCGCTGGCTTTCCAACGCCCGGA | | | |
| | TGACTCACTATCAGGCCTTGCTTTTGGACAC | | | |
| | GGACCGGGTCCAGTTCGGACCGGTGGTAGCC | | | |
| | CTGAACCCGGCTACGCTGCTCCCACTGCCTG | | | |
| | AGGAAGGGCTGCAACACAACTGCCTTGATAT | | | |
| | CCTGGCCGAAGCCCACGGAACCCGACCCGAC | | | |
| | CTAACGGACCAGCCGCTCCCAGACGCCGACC | | | |
| | ACACCTGGTACACGGATGGAAGCAGTCTCTT | | | |
| | ACAAGAGGGACAGCGTAAGGCGGGAGCTGCG | | | |
| | GTGACCACCGAGACCGAGGTAATCTGGGCTA | | | |
| | AAGCCCTGCCAGCCGGGACATCCGCTCAGCG | | | |
| | GGCTGAACTGATAGCACTCACCCAGGCCCTA | | | |
| | AAGATGGCAGAAGGTAAGAAGCTAAATGTTT | | | |
| | ATACTGATAGCCGTTATGCTTTTGCTACTGC | | | |
| | CCATATCCATGGAGAAATATACAGAAGGCGT | | | |
| | GGGTGGCTCACATCAGAAGGCAAAGAGATCA | | | |
| | AAAATAAAGACGAGATCTTGGCCCTACTAAA | | | |
| | AGCCCTCTTTCTGCCCAAAAGACTTAGCATA | | | |
| | ATCCATTGTCCAGGACATCAAAAGGGACACA | | | |
| | GCGCCGAGGCTAGAGGCAACCGGATGGCTGA | | | |
| | CCAAGCGGCCCGAAAGGCAGCCATCACAGAG | | | |
| | ACTCCAGACACCTCTACCCTCCTCATAGAAA | | | |
| | ATTCATCACCCTCTGGCGGCTCAAAAAGAAC | | | |
| | CGCCGACGGCAGCGAATTCGAGCCCAAGAAG | | | |
| | AAGAGGAAAGTCGGAAGCGGAGCTACTAACT | | | |
| | TCAGCCTGCTGAAGCAGGCTGGAGACGTGGA | | | |
| | GGAGAACCCTGGACCTATGGTGAGCAAGGGC | | | |
| | GAGGAGCTGTTCACCGGGGTGGTGCCCATCC | | | |
| | TGGTCGAGCTGGACGGCGACGTAAACGGCCA | | | |
| | CAAGTTCAGCGTGTCCGGCGAGGGCGAGGGC | | | |
| | GATGCCACCTACGGCAAGCTGACCCTGAAGT | | | |
| | TCATCTGCACCACCGGCAAGCTGCCCGTGCC | | | |
| | CTGGCCCACCCTCGTGACCACCCTGACCTAT | | | |
| | GGAGTGCAGTGCTTCAGCCGCTACCCCGACC | | | |
| | ACATGAAGCAGCACGACTTCTTCAAGTCCGC | | | |
| | CATGCCCGAAGGCTACGTCCAGGAGCGCACC | | | |
| | ATCTTCTTCAAGGACGACGGCAACTACAAGA | | | |
| | CCCGCGCCGAGGTGAAGTTCGAGGGCGACAC | | | |
| | CCTGGTGAACCGCATCGAGCTGAAGGGCATC | | | |
| | GACTTCAAGGAGGACGGCAACATCCTGGGGC | | | |
| | ACAAGCTGGAGTACAACTACAACAGCCACAA | | | |
| | CGTCTATATCATGGCCGACAAGCAGAAGAAC | | | |
| | GGCATCAAGGTGAACTTCAAGATCCGCCACA | | | |
| | ACATCGAGGACGGCAGCGTGCAGCTCGCCGA | | | |
| | CCACTACCAGCAGAACACCCCCATCGGCGAC | | | |
| | GGCCCCGTGCTGCTGCCCGACAACCACTACC | | | |
| | TGAGCACCCAGTCCGCCCTGAGCAAAGACCC | | | |
| | CAACGAGAAGCGCGATCACATGGTCCTGCTG | | | |
| | GAGTTCGTGACCGCCGCCGGGATCACTCTCG | | | |
| | GCATGGACGAGCTGTACAAGTAA | | | |
| bpNLS-nCas9 | ATGAAACGGACAGCCGACGGAAGCGAGTTCG | 92 | MKRTADGSEFESPKKKRKVDKKYSIGLDIG | 93 |
| (H840A) pt. | AGTCACCAAAGAAGAAGCGGAAAGTCGACAA | | INSVGWAVITDEYKVPSKKFKVLGNTDRHS | |
| 1-32 AA | GAAGTACAGCATCGGCCTGGACATCGGCACC | | IKKNLIGALLFDSGETAEATRLKRTARRRY | |
| linker-MMLV | AACTCTGTGGGCTGGGCCGTGATCACCGACG | | TRRKNRICYLQEIFSNEMAKVDDSFFHRLE | |
| RT-32 AA | AGTACAAGGTGCCCAGCAAGAAATTCAAGGT | | ESFLVEEDKKHERHPIFGNIVDEVAYHEKY | |
| linker- | GCTGGGCAACACCGACCGGCACAGCATCAAG | | PTIYHLRKKLVDSTDKADLRLIYLALAHMI | |
| nCas9 (H840A) | AAGAACCTGATCGGAGCCCTGCTGTTCGACA | | KFRGHFLIEGDLNPDNSDVDKLFIQLVQTY | |
| pt. 2-4 AA | GCGGCGAAACAGCCGAGGCCACCCGGCTGAA | | NQLFEENPINASGVDAKAILSARLSKSRRL | |
| linker-bpNLS- | GAGAACCGCCAGAAGAAGATACACCAGACGG | | ENLIAQLPGEKKNGLFGNLIALSLGLIPNE | |
| P2A-eGPP-- | AAGAACCGGATCTGCTATCTGCAAGAGATCT | | KSNFDLAEDAKLQLSKDTYDDDLDNLLAQI | |
| MMLV-RT | TCAGCAACGAGATGGCCAAGGTGGACGACAG | | GDQYADLFLAAKNLSDAILLSDILRVNTEI | |
| inlaid at | CTTCTTCCACAGACTGGAAGAGTCCTTCCTG | | TKAPLSASMIKRYDEHHQDLTLLKALVRQQ | |
| | GTGGAAGAGGATAAGAAGCACGAGCGGCACC | | LPEKYKEIFFDQSKNGYAGYIDGGASQEEF | |
| | CCATCTTCGGCAACATCGTGGACGAGGTGGC | | YKFIKPILEKMDGTEELLVKENREDLLRKQ | |
| | CTACCACGAGAAGTACCCCACCATCTACCAC | | RTFDNGSIPHQIHLGELHAILRRQEDFYPF | |
| | CTGAGAAAGAAACTGGTGGACAGCACCGACA | | LKDNREKIEKILIFRIPYYVGPLARGNSRE | |
| | AGGCCGACCTGCGGCTGATCTATCTGGCCCT | | AWMTRKSEETITPWNFEEVVDKGASAQSFI | |
| | GGCCCACATGATCAAGTTCCGGGGCCACTTC | | ERMTNFDKNLPNEKVLPKHSLLYEYFTVYN | |
| | CTGATCGAGGGCGACCTGAACCCCGACAACA | | ELTKVKYVTEGMRKPAFLSGEQKKAIVDLL | |
| | GCGACGTGGACAAGCTGTTCATCCAGCTGGT | | FKTNRKVTVKQLKEDYFKKIECFDSVEISG | |
| | GCAGACCTACAACCAGCTGTTCGAGGAAAAC | | VEDRFNASLGTYHDLLKIIKDKDFLDNEEN | |
| | CCCATCAACGCCAGCGGCGTGGACGCCAAGG | | EDILEDIVLTLTLFEDREMIEERLKTYAHL | |
| | CCATCCTGTCTGCCAGACTGAGCAAGAGCAG | | FDDKVMKQLKRRRYTGWGRLSRKLINGIRD | |
| | ACGGCTGGAAAATCTGATCGCCCAGCTGCCC | | KQSGKTILDFLKSDGFANRNFMQLIHDDSL | |
| | GGCGAGAAGAAGAATGGCCTGTTCGGAAACC | | TFKEDIQKAQVSGQGDSLHEHIANLAGSPA | |
| | TGATTGCCCTGAGCCTGGGCCTGACCCCCAA | | IKKGILQTVKVVDELVKVMGRHKPENIVIE | |
| | CTTCAAGAGCAACTTCGACCTGGCCGAGGAT | | MARENQTTQKGQKNSRERMKRIEEGIKELG | |
| | GCCAAACTGCAGCTGAGCAAGGACACCTACG | | SQILKEHPVENTQLQNEKLYLYYLQNGRDM | |
| | ACGACGACCTGGACAACCTGCTGGCCCAGAT | | YVDQELDINRLSDYDVDAIVPQSFLKDDSI | |
| | CGGCGACCAGTACGCCGACCTGTTTCTGGCC | | DNKVLTRSDKNRGKSDNVPSEEVVKKMKNY | |
| | GCCAAGAACCTGTCCGACGCCATCCTGCTGA | | WRQLLNAKLITQRKFDNLTKAERGGLSELD | |
| | GCGACATCCTGAGAGTGAACACCGAGATCAC | | KAGFIKRQLVETRQITKHVAQILDSRMNTK | |
| | CAAGGCCCCCCTGAGCGCCTCTATGATCAAG | | YDENDKLIREVKVITLKSKLVSDERKDFQF | |
| | AGATACGACGAGCACCACCAGGACCTGACCC | | YKVREINNYHHAHDAYLNAVVGIALIKKYP | |
| | TGCTGAAAGCTCTCGTGCGGCAGCAGCTGCC | | KLESEFVYGDYKVYDVRKMIAKSEQEIGKA | |
| | TGAGAAGTACAAAGAGATTTTCTTCGACCAG | | TAKYFFYSNIMNFFKTEITLANGGGSSGGS | |
| | AGCAAGAACGGCTACGCCGGCTACATTGACG | | SGSETPGTSESATPESSGGSSGGSSTLNIE | |
| | GCGGAGCCAGCCAGGAAGAGTTCTACAAGTT | | DEYRLHETSKEPDVSLGSTWLSDFPQAWAE | |
| | CATCAAGCCCATCCTGGAAAAGATGGACGGC | | TGGMGLAVRQAPLIIPLKATSTPVSIKQYP | |
| | ACCGAGGAACTGCTCGTGAAGCTGAACAGAG | | MSQEARLGIKPHIQRLLDQGILVPCQSPWN | |
| | AGGACCTGCTGCGGAAGCAGCGGACCTTCGA | | TPLLPVKKPGTNDYRPVQDLREVNKRVEDI | |
| | CAACGGCAGCATCCCCCACCAGATCCACCTG | | HPTVPNPYNLLSGLPPSHQWYTVLDLKDAF | |
| | GGAGAGCTGCACGCCATTCTGCGGCGGCAGG | | FCLRLHPTSQPLFAFEWRDPEMGISGQLTW | |
| | AAGATTTTTACCCATTCCTGAAGGACAACCG | | TRLPQGFKNSPTLFNEALHRDLADFRIQHP | |
| | GGAAAAGATCGAGAAGATCCTGACCTTCCGC | | DLILLQYVDDLLLAATSELDCQQGTRALLQ | |
| | ATCCCCTACTACGTGGGCCCTCTGGCCAGGG | | TLGNLGYRASAKKAQICQKQVKYLGYLLKE | |
| | GAAACAGCAGATTCGCCTGGATGACCAGAAA | | GQRWLTEARKETVMGQPTPKTPRQLREFLG | |
| | GAGCGAGGAAACCATCACCCCCTGGAACTTC | | KAGFCRLFIPGFAEMAAPLYPLTKPGTLEN | |
| | GAGGAAGTGGTGGACAAGGGCGCTTCCGCCC | | WGPDQQKAYQEIKQALLTAPALGLPDLTKP | |
| | AGAGCTTCATCGAGCGGATGACCAACTTCGA | | FELFVDEKQGYAKGVLTQKLGPWRRPVAYL | |
| | TAAGAACCTGCCCAACGAGAAGGTGCTGCCC | | SKKLDPVAAGWPPCLRMVAAIAVLTKDAGK | |
| | AAGCACAGCCTGCTGTACGAGTACTTCACCG | | LIMGQPLVILAPHAVEALVKQPPDRWLSNA | |
| | TGTATAACGAGCTGACCAAAGTGAAATACGT | | RMTHYQALLLDTDRVQFGPVVALNPATLLP | |
| | GACCGAGGGAATGAGAAAGCCCGCCTTCCTG | | LPEEGLQHNCLDILAEAHGTRPDLTDQPLP | |
| | AGCGGCGAGCAGAAAAAGGCCATCGTGGACC | | DADHTWYTDGSSLLQEGQRKAGAAVTTETE | |
| | TGCTGTTCAAGACCAACCGGAAAGTGACCGT | | VIWAKALPAGTSAQRAELIALTQALKMAEG | |
| | GAAGCAGCTGAAAGAGGACTACTTCAAGAAA | | KKLNVYTDSRYAFATAHIHGEIYRRRGWLT | |
| | ATCGAGTGCTTCGACTCCGTGGAAATCTCCG | | SEGKEIKNKDEILALLKALFLPKRLSIIHC | |
| | GCGTGGAAGATCGGTTCAACGCCTCCCTGGG | | PGHQKGHSAEARGNRMADQAARKAAITETP | |
| | CACATACCACGATCTGCTGAAAATTATCAAG | | DTSTLLIENSSPSGGSSGGSSGSETPGTSE | |
| | GACAAGGACTTCCTGGACAATGAGGAAAACG | | SATPESSGGSSGGSEIRKRPLIETNGETGE | |
| | AGGACATTCTGGAAGATATCGTGCTGACCCT | | IVWDKGRDFATVRKVLSMPQVNIVKKTEVQ | |
| | GACACTGTTTGAGGACAGAGAGATGATCGAG | | TGGFSKESILPKRNSDKLIARKKDWDPKKY | |
| | GAACGGCTGAAAACCTATGCCCACCTGTTCG | | GGFDSPTVAYSVLVVAKVEKGKSKKLKSVK | |
| | ACGACAAAGTGATGAAGCAGCTGAAGCGGCG | | ELLGITIMERSSFEKNPIDFLEAKGYKEVK | |
| | GAGATACACCGGCTGGGGCÄGGCTGAGCCGG | | KDLIIKLPKYSLFELENGRKRMLASAGELQ | |
| | AAGCTGATCAACGGCATCCGGGACAAGCAGT | | KGNELALPSKYVNFLYLASHYEKLKGSPED | |
| | CCGGCAAGACAATCCTGGATTTCCTGAAGTC | | NEQKQLFVEQHKHYLDEIIEQISEFSKRVI | |
| | CGACGGCTTCGCCAACAGAAACTTCATGCAG | | LADANLDKVLSAYNKHRDKPIREQAENIIH | |
| | CTGATCCACGACGACAGCCTGACCTTTAAAG | | LFTLTNLGAPAAFKYFDTTIDRKRYTSTKE | |
| | AGGACATCCAGAAAGCCCAGGTGTCCGGCCA | | VLDATLIHQSITGLYETRIDLSQLGGDSGG | |
| | GGGCGATAGCCTGCACGAGCACATTGCCAAT | | SKRTADGSEFEPKKKRKVGSGATNFSLLKQ | |
| | CTGGCCGGCAGCCCCGCCATTAAGAAGGGCA | | AGDVEENPGPMVSKGEELFTGVVPILVELD | |
| | TCCTGCAGACAGTGAAGGTGGTGGACGAGCT | | GDVNGHKFSVSGEGEGDATYGKLTLKPICT | |
| | CGTGAAAGTGATGGGCCGGCACAAGCCCGAG | | TGKLPVPWPTLVTTLTYGVQCFSRYPDHMK | |
| | AACATCGTGATCGAAATGGCCAGAGAGAACC | | QHDFFKSAMPEGYVQERTIFFKDDGNYKTR | |
| | AGACCACCCAGAAGGGACAGAAGAACAGCCG | | AEVKFEGDTLVNRIELKGIDFKEDGNILGH | |
| | CGAGAGAATGAAGCGGATCGAAGAGGGCATC | | KLEYNYNSHNVYIMADKQKNGIKVNFKIRH | |
| | AAAGAGCTGGGCAGCCAGATCCTGAAAGAAC | | NIEDGSVQLADHYQQNTPIGDGPVLLPDNH | |
| | ACCCCGTGGAAAACACCCAGCTGCAGAACGA | | YLSTQSALSKDPNEKRDHMVLLEFVTAAGI | |
| | GAAGCTGTACCTGTACTACCTGCAGAATGGG | | TLGMDELYK* | |
| | CGGGATATGTACGTGGACCAGGAACTGGACA | | | |
| | TCAACCGGCTGTCCGACTACGATGTGGACGC | | | |
| | TATCGTGCCTCAGAGCTTTCTGAAGGACGAC | | | |
| | TCCATCGACAACAAGGTGCTGACCAGAAGCG | | | |
| | ACAAGAACCGGGGCAAGAGCGACAACGTGCC | | | |
| | CTCCGAAGAGGTCGTGAAGAAGATGAAGAAC | | | |
| | TACTGGCGGCAGCTGCTGAACGCCAAGCTGA | | | |
| | TTACCCAGAGAAAGTTCGACAATCTGACCAA | | | |
| | GGCCGAGAGAGGCGGCCTGAGCGAACTGGAT | | | |
| | AAGGCCGGCTTCATCAAGAGACAGCTGGTGG | | | |
| | AAACCCGGCAGATCACAAAGCACGTGGCACA | | | |
| | GATCCTGGACTCCCGGATGAACACTAAGTAC | | | |
| | GACGAGAATGACAAGCTGATCCGGGAAGTGA | | | |
| | AAGTGATCACCCTGAAGTCCAAGCTGGTGTC | | | |
| | CGATTTCCGGAAGGATTTCCAGTTTTACAAA | | | |
| | GTGCGCGAGATCAACAACTACCACCACGCCC | | | |
| | ACGACGCCTACCTGAACGCCGTCGTGGGAAC | | | |
| | CGCCCTGATCAAAAAGTACCCTAAGCTGGAA | | | |
| | AGCGAGTTCGTGTACGGCGACTACAAGGTGT | | | |
| | ACGACGTGCGGAAGATGATCGCCAAGAGCGA | | | |
| | GCAGGAAATCGGCAAGGCTACCCCCAAGTAC | | | |
| | TTCTTCTACAGCAACATCATGAACTTTTTCA | | | |
| | AGACCGAGATTACCCTGGCCAACGGCGGAGG | | | |
| | ATCTAGCGGAGGATCCTCTGGAAGCGAGACA | | | |
| | CCAGGCACAAGCGAGTCCGCCACACCAGAGA | | | |
| | GCTCCGGCGGCTCCTCCGGAGGATCCTCTAC | | | |
| | CCTAAATATAGAAGATGAGTATCGGCTACAT | | | |
| | GAGACCTCAAAAGAGCCAGATGTTTCTCTAG | | | |
| | GGTCCACATGGCTGTCTGATTTTCCTCAGGC | | | |
| | CTGGGCGGAAACCGGGGGCATGGGACTGGCA | | | |
| | GTTCGCCAAGCTCCTCTGATCATACCTCTGA | | | |
| | AAGCAACCTCTACCCCCGTGTCCATAAAACA | | | |
| | ATACCCCATGTCACAAGAAGCCAGACTGGGG | | | |
| | ATCAAGCCCCACATACAGAGACTGTTGGACC | | | |
| | AGGGAATACTGGTACCCTGCCAGTCCCCCTG | | | |
| | GAACACGCCCCTGCTACCCGTTAAGAAACCA | | | |
| | GGGACTAATGATTATAGGCCTGTCCAGGATC | | | |
| | TGAGAGAAGTCAACAAGCGGGTGGAAGACAT | | | |
| | CCACCCCACCGTGCCCAACCCTTACAACCTC | | | |
| | TTGAGCGGGCTCCCACCGTCCCACCAGTGGT | | | |
| | ACACTGTGCTTGATTTAAAGGATGCCTTTTT | | | |
| | CTGCCTGAGACTCCACCCCACCAGTCAGCCT | | | |
| | CTCTTCGCCTTTGAGTGGAGAGATCCAGAGA | | | |
| | TGGGAATCTCAGGACAATTGACCTGGACCAG | | | |
| | ACTCCCACAGGGTTTCAAAAACAGTCCCACC | | | |
| | CTGTTTAATGAGGCACTGCACAGAGACCTAG | | | |
| | CAGACTTCCGGATCCAGCACCCAGACTTGAT | | | |
| | CCTGCTACAGTACGTGGATGACTTACTGCTG | | | |
| | GCCGCCACTTCTGAGCTAGACTGCCAACAAG | | | |
| | GTACTCGGGCCCTGTTACAAACCCTAGGGAA | | | |
| | CCTCGGGTATCGGGCCTCGGCCAAGAAAGCC | | | |
| | CAAATTTGCCAGAAACAGGTCAAGTATCTGG | | | |
| | GGTATCTTCTAAAAGAGGGTCAGAGATGGCT | | | |
| | GACTGAGGCCAGAAAAGAGACTGTGATGGGG | | | |
| | CAGCCTACTCCGAAGACCCCTCGACAACTAA | | | |
| | GGGAGTTCCTAGGGAAGGCAGGCTTCTGTCG | | | |
| | CCTCTTCATCCCTGGGTTTGCAGAAATGGCA | | | |
| | GCCCCCCTGTACCCTCTCACCAAACCGGGGA | | | |
| | CTCTGTTTAATTGGGGCCCAGACCAACAAAA | | | |
| | GGCCTATCAAGAAATCAAGCAAGCTCTTCTA | | | |
| | ACTGCCCCAGCCCTGGGGTTGCCAGATTTGA | | | |
| | CTAAGCCCTTTGAACTCTTTGTCGACGAGAA | | | |
| | GCAGGGCTACGCCAAAGGTGTCCTAACGCAA | | | |
| | AAACTGGGACCTTGGCGTCGGCCGGTGGCCT | | | |
| | ACCTGTCCAAAAAGCTAGACCCAGTAGCAGC | | | |
| | TGGGTGGCCCCCTTGCCTACGGATGGTAGCA | | | |
| | GCCATTGCCGTACTGACAAAGGATGCAGGCA | | | |
| | AGCTAACCATGGGACAGCCACTAGTCATTCT | | | |
| | GGCCCCCCATGCAGTAGAGGCACTAGTCAZA | | | |
| | CAACCCCCCGACCGCTGGCTTTCCAACGCCC | | | |
| | GGATGACTCACTATCAGGCCTTGCTTTTGGA | | | |
| | CACGGACCGGGTCCAGTTCGGACCGGTGGTA | | | |
| | GCCCTGAACCCGGCTACGCTGCTCCCACTGC | | | |
| | CTGAGGAAGGGCTGCAACACAACTGCCTTGA | | | |
| | TATCCTGGCCGAAGCCCACGGAACCCGACCC | | | |
| | GACCTAACGGACCAGCCGCTCCCAGACGCCG | | | |
| | ACCACACCTGGTACACGGATGGAAGCAGTCT | | | |
| | CTTACAAGAGGGACAGCGTAAGGCGGGAGCT | | | |
| | GCGGTGACCACCGAGACCGAGGTAATCTGGG | | | |
| | CTAAAGCCCTGCCAGCCGGGACATCCGCTCA | | | |
| | GCGGGCTGAACTGATAGCACTCACCCAGGCC | | | |
| | CTAAAGATGGCAGAAGGTAAGAAGCTAAATG | | | |
| | TTTATACTGATAGCCGTTATGCTTTTGCTAC | | | |
| | TGCCCATATCCATGGAGAAATATACAGAAGG | | | |
| | CGTGGGTGGCTCACATCAGAAGGCAAAGAGA | | | |
| | TCAAAAATAAAGACGAGATCTTGGCCCTACT | | | |
| | AAAAGCCCTCTTTCTGCCCAAAAGACTTAGC | | | |
| | ATAATCCATTGTCCAGGACATCAAAAGGGAC | | | |
| | ACAGCGCCGAGGCTAGAGGCAACCGGATGGC | | | |
| | TGACCAAGCGGCCCGAAAGGCAGCCATCACA | | | |
| | GAGACTCCAGACACCTCTACCCTCCTCATAG | | | |
| | AAAATTCATCACCCTCCGGAGGATCTAGCGG | | | |
| | AGGCTCCTCTGGCTCTGAGACACCTGGCACA | | | |
| | AGCGAGAGCGCAACACCTGAAAGCAGCGGGG | | | |
| | GCAGCAGCGGGGGGTCAGAGATCCGGAAGCG | | | |
| | GCCTCTGATCGAGACAAACGGCGAAACCGGG | | | |
| | GAGATCGTGTGGGATAAGGGCCGGGATTTTG | | | |
| | CCACCGTGCGGAAAGTGCTGAGCATGCCCCA | | | |
| | AGTGAATATCGTGAAAAAGACCGAGGTGCAG | | | |
| | ACAGGCGGCTTCAGCAAAGAGTCTATCCTGC | | | |
| | CCAAGAGGAACAGCGATAAGCTGATCGCCAG | | | |
| | AAAGAAGGACTGGGACCCTAAGAAGTACGGC | | | |
| | GGCTTCGACAGCCCCACCGTGGCCTATTCTG | | | |
| | TGCTGGTGGTGGCCAAAGTGGAAAAGGGCAA | | | |
| | GTCCAAGAAACTGAAGAGTGTGAAAGAGCTG | | | |
| | CTGGGGATCACCATCATGGAAAGAAGCAGCT | | | |
| | TCGAGAAGAATCCCATCGACTTTCTGGAAGC | | | |
| | CAAGGGCTACAAAGAAGTGAAAAAGGACCTG | | | |
| | ATCATCAAGCTGCCTAAGTACTCCCTGTTCG | | | |
| | AGCTGGAAAACGGCCGGAAGAGAATGCTGGC | | | |
| | CTCTGCCGGCGAACTGCAGAAGGGAAACGAA | | | |
| | CTGGCCCTGCCCTCCAAATATGTGAACTTCC | | | |
| | TGTACCTGGCCAGCCACTATGAGAAGCTGAA | | | |
| | GGGCTCCCCCGAGGATAATGAGCAGAAACAG | | | |
| | CTGTTTGTGGAACAGCACAAGCACTACCTGG | | | |
| | ACGAGATCATCGAGCAGATCAGCGAGTTCTC | | | |
| | CAAGAGAGTGATCCTGGCCGACGCTAATCTG | | | |
| | GACAAAGTGCTGTCCGCCTACAACAAGCACC | | | |
| | GGGATAAGCCCATCAGAGAGCAGGCCGAGAA | | | |
| | TATCATCCACCTGTTTACCCTGACCAATCTG | | | |
| | GGAGCCCCTGCCGCCTTCAAGTACTTTGACA | | | |
| | CCACCATCGACCGGAAGAGGTACACCAGCAC | | | |
| | CAAAGAGGTGCTGGACGCCACCCTGATCCAC | | | |
| | CAGAGCATCACCGGCCTGTACGAGACACGGA | | | |
| | TCGACCTGTCTCAGCTGGGAGGTGACTCTGG | | | |
| | CGGCTCAAAAAGAACCGCCGACGGCAGCGAA | | | |
| | TTCGAGCCCAAGAAGAAGAGGAAAGTCGGAA | | | |
| | GCGGAGCTACTAACTTCAGCCTGCTGAAGCA | | | |
| | GGCTGGAGACGTGGAGGAGAACCCTGGACCT | | | |
| | ATGGTGAGCAAGGGCGAGGAGCTGTTCACCG | | | |
| | GGGTGGTGCCCATCCTGGTCGAGCTGGACGG | | | |
| | CGACGTAAACGGCCACAAGTTCAGCGTGTCC | | | |
| | GGCGAGGGCGAGGGCGATGCCACCTACGGCA | | | |
| | AGCTGACCCTGAAGTTCATCTGCACCACCGG | | | |
| | CAAGCTGCCCGTGCCCTGGCCCACCCTCGTG | | | |
| | ACCACCCTGACCTATGGAGTGCAGTGCTTCA | | | |
| | GCCGCTACCCCGACCACATGAAGCAGCACGA | | | |
| | CTTCTTCAAGTCCGCCATGCCCGAAGGCTAC | | | |
| | GTCCAGGAGCGCACCATCTTCTTCAAGGACG | | | |
| | ACGGCAACTACAAGACCCGCGCCGAGGTGAA | | | |
| | GTTCGAGGGCGACACCCTGGTGAACCGCATC | | | |
| | GAGCTGAAGGGCATCGACTTCAAGGAGGACG | | | |
| | GCAACATCCTGGGGCACAAGCTGGAGTACAA | | | |
| | CTACAACAGCCACAACGTCTATATCATGGCC | | | |
| | GACAAGCAGAAGAACGGCATCAAGGTGAACT | | | |
| | TCAAGATCCGCCACAACATCGAGGACGGCAG | | | |
| | CGTGCAGCTCGCCGACCACTACCAGCAGAAC | | | |
| | ACCCCCATCGGCGACGGCCCCGTGCTGCTGC | | | |
| | CCGACAACCACTACCTGAGCACCCAGTCCGC | | | |
| | CCTGAGCAAAGACCCCAACGAGAAGCGCGAT | | | |
| | CACATGGTCCTGCTGGAGTTCGTGACCGCCG | | | |
| | CCGGGATCACTCTCGGCATGGACGAGCTGTA | | | |
| | CAAGTAA | | | |
| bpNLS- | ATGAAACGGACAGCCGACGGAAGCGAGTTCG | 94 | MKRTADGSEFESPKKKRKVKRNYILGLDIG | 95 |
| nSaCas9 | AGTCACCAAAGAAGAAGCGGAAAGTCAAACG | | ITSVGYGIIDYETRDVIDAGVRLFKEANVE | |
| (N580A) | GAACTACATCCTGGGGCTTGACATTGGGATA | | NNEGRRSKRGARRLKRRRRHRIQRVKKLLF | |
| KKH-XTEN- | ACCAGCGTTGGCTACGGAATTATTGATTATG | | DYNLLIDHSELSGINPYEARVKGLSQKLSE | |
| MMLV | AGACACGCGATGTGATTGACGCCGGGGTTAG | | EEFSAALLHLAKRRGVHNVNEVEEDTGNEL | |
| RT-4 AA | GCTGTTCAAAGAGGCCAACGTTGAAAACAAC | | STKEQISRNSKALEEKYVAELQLERLKKDG | |
| linker- | GAGGGAAGACGGAGTAAGCGCGGAGCAAGAA | | EVRGSINREKTSDYVKEAKQLLKVQKAYHQ | |
| bpNLS-P2A | GACTCAAGCGCAGACGGAGACATCGGATTCA | | LDQSFIDTYIDLLETRRTYYEGPGEGSPFG | |
| -eGFP | GAGGGTGAAAAAGCTGCTCTTCGATTACAAT | | WKDIKEWYEMLMGHCTYFPEELRSVKYAYN | |
| | CTCCTGACCGATCATAGTGAGCTGAGCGGAA | | ADLYNALNDINNLVITRDENEKLEYYEKFQ | |
| | TCAACCCCTACGAGGCGCGAGTGAAAGGGCT | | IIENVFKQKKKPTLKQIAKEILVNEEDIKG | |
| | TTCCCAGAAGCTGTCCGAAGAGGAGTTCTCC | | YRVTSTGKPEFTNLKVYHDIKDITARKEII | |
| | GCCGCGTTGCTGCACCTGGCCAAACGGAGGG | | ENAELLDQIAKILTIYQSSEDIQEELTNLN | |
| | GGGTTCACAATGTAAACGAAGTGGAGGAGGA | | SELTQEEIEQISNLKGYTGTHNLSLKAINL | |
| | CACGGGCAATGAACTTAGTACGAAAGAACAG | | ILDELWHTNDNQIAIFNRLKLVPKKVDLSQ | |
| | ATCAGTAGGAACTCTAAGGCTCTCGAAGAGA | | QKEIPTTLVDDFILSPVVKRSFIQSIKVIN | |
| | AATACGTCGCTGAGTTGCAGCTTGAGAGACT | | AIIKKYGLENDIIIELAREKNSKDAQKMIN | |
| | GAAAAAAGACGGCGAAGTACGCGGATCTATT | | EMQKRNRQTNERIEEIIRTIGKENAKYLIE | |
| | AATAGGTTCAAGACTTCAGATTACGTAAAGG | | KIKLHDMQEGKCLYSLEAIPLEDLLNNPEN | |
| | AAGCCAAGCAGCTCCTGAAAGTACAGAAAGC | | YEVDHIIPRSVSFDNSENNKVLVKQEEASK | |
| | GTACCATCAGCTCGATCAGAGCTTCATCGAT | | KGNRTPFQYLSSSDSKISYETFKKHILNLA | |
| | ACCTACATAGATTTGCTGGAGACACGGAGGA | | KGKGRISKTKKEYLLEERDINRESVQKDPI | |
| | CATACTACGAGGGCCCAGGGGAAGGATCTCC | | NRNLVDTRYATRGLMNLLRSYFRVNNLDVK | |
| | TTTTGGGTGGAAGGACATCAAGGAATGGTAC | | VKSINGGFTSFLRRKWKFKKERNKGYKHHA | |
| | GAGATGCTTATGGGACATTGTACATATTTTC | | EDALIIANADFIFKEWKKLDKAKKVMENQM | |
| | CGGAGGAGCTCAGGAGCGTCAAGTACGCCTA | | FEEKQAESMPEIETEQEYKEIFITPHQIKH | |
| | CAATGCCGACCTGTACAATGCCCTCAATGAC | | IKDFKDYKYSHRVDKKPNRKLINDTLYSTR | |
| | CTCAATAACCTCGTGATTACCAGGGACGAGA | | KDDKGNTLIVNNLNGLYDKDNDKLKKLINK | |
| | ACGAGAAGCTGGAGTACTATGAAAAGTTCCA | | SPEKLLMYHHDPQTYQKLKLIMEQYGDEKN | |
| | GATTATCGAGAATGTGTTTAAGCAGAAGAAG | | PLYKYYEETGNYLTKYSKKDNGPVIKKIKY | |
| | AAGCCGACACTTAAGCAGATTGCAAAGGAAA | | YGNKLNAHLDITDDYPNSRNKVVKLSLKPY | |
| | TCCTCGTGAATGAGGAAGATATCAAGGGATA | | RFDVYLDNGVYKFVTVKNLDVIKKENYYEV | |
| | CAGAGTGACAAGTACAGGCAAGCCCGAGTTC | | NSKCYEEAKKLKKISNQAEFIASFYKNDLI | |
| | ACAAATCTGAAGGTGTACCACGATATTAAGG | | KINGELYRVIGVNNDLLNRIEVNMIDITYR | |
| | ACATAACCGCACGAAAGGAGATAATCGAAAA | | EYLENMNDKRPPHIIKTIASKTQSIKKYST | |
| | CGCTGAGCTCCTCGATCAGATCGCAAAAATT | | DILGNLYEVKSKKHPQIIKKGSGGSSGGSS | |
| | CTTACCATCTACCAGTCTAGTGAGGACATTC | | GSETPGTSESATPESSGGSSGGSSTLNIED | |
| | AGGAGGAACTGACTAATCTGAACAGTGAGCT | | EYRLHETSKEPDVSLGSTWLSDFPQAWAST | |
| | CACCCAAGAGGAAATTGAGCAGATTTCAAAC | | GGMGLAVRQAPLIIPLKATSTPVSIKQYPM | |
| | CTGAAAGGCTACACCGGGACGCACAATCTGA | | SQEARLGIKPHIQRLLDQGILVPCQSPWNT | |
| | GCCTCAAAGCAATCAACCTCATTCTGGATGA | | PLLPVKKPGTNDYRPVQDLREVNKRVEDIH | |
| | ACTTTGGCACACAAATGACAACCAAATTGCC | | PTVPNPYNLLSGLPPSHQWYTVLDLKDAFF | |
| | ATATTCAACCGCCTGAAACTGCTGCCAAAAA | | CLRLHPTSQPLFAFEWRDPEMGISGQLIWT | |
| | AAGTGGATCTGTCACAGCAAAAGGAAATCCC | | RLPQGFKNSPTLFNEALHRDLADFRIQHPD | |
| | TACAACCTTGGTTGACGATTTTATTCTGTCC | | LILLQYVDDLLLAATSELDCQQGTRALLQT | |
| | CCCGTTGTCAAGCGGAGCTTCATCCAGTCAA | | LGNLGYRASAKKAQICQKQVKYLGYLLKEG | |
| | TCAAGGTGATCAATGCCATCATTAAAAAATA | | QRWLTEARKETVMGQPTPKTPRQLREFLGK | |
| | CGGATTGCCAAACGATATAATTATCGAGCTT | | AGFCRLFIPGFAEMAAPLYPLTKPGTLFNW | |
| | GCACGAGAGAAGAACTCAAAGGACGCCCAGA | | GPDQQKAYQEIKQALLTAPALGLPDLTKPF | |
| | AGATGATTAACGAAATGCAGAAGCGCAACCG | | ELFVDEKQGYAKGVLTQKLGPWRRPVAYLS | |
| | CCAGACAAACGAACGCATAGAGGAAATTATA | | KKLDPVAAGWPPCLRMVAAIAVLTKDAGKL | |
| | AGAACAACCGGCAAAGAGAATGCCAAGTATC | | TMGQPLVILAPHAVEALVKQPPDRWLSNAR | |
| | TGATCGAGAAAATCAAGCTGCACGACATGCA | | MTHYQALLLDTDRVQFGPVVALNPATLLPL | |
| | AGAAGGCAAGTGCCTGTACTCTCTGGAAGCT | | PEEGLQHNCLDILAEAHGTRPDLTDQPLPD | |
| | ATCCCACTCGAAGATCTGCTGAATAATCCAT | | ADHTWYTDGSSLLQEGQRKAGAAVTTETEV | |
| | TCAATTACGAGGTGGACCACATCATCCCTAG | | IWAKALPAGTSAQRAELIALTQALKMAEGK | |
| | ATCCGTAAGCTTTGACAATTCCTTCAATAAC | | KLNVYTDSRYAFATAHINGETYRRRGWLTS | |
| | AAAGTTCTGGTTAAACAGGAGGAAGCCTCTA | | EGKEIKNKDEILALLKALFLPKRLSIIHCP | |
| | AAAAAGGGAACCGGACCCCGTTCCAGTACCT | | GHQKGHSARARGNRMADQAARKAAITETPD | |
| | GAGCTCCAGTGACAGCAAGATTAGCTACGAG | | TSTLLIENSSPSGGSKRTADGSEFEPKKKR | |
| | ACTTTTAAGAAACATATTCTGAATCTGGCCA | | KVGSGATNFSLLKQAGDVEENPGPMVSKGE | |
| | AAGGCAAAGGCAGGATCAGCAAGACCAAGAA | | ELFTGVVPILVELDGDVNGHKFSVSGEGEG | |
| | GGAGTACCTCCTCGAAGAACGCGACATTAAC | | DATYGKLTLKFICTTGKLPVPWPTLVTTLT | |
| | AGATTTAGTGTGCAGAAAGATTTCATCAACC | | YGVQCFSRYPDHMKQHDFFKSAMPEGYVQE | |
| | GAAACCTTGTCGATACTCGGTACGCCACGAG | | RTIFFKDDGNYKTRAEVKFEGDTLVNRIEL | |
| | AGGCCTGATGAATCTCCTCAGGAGCTACTTC | | KGIDFKEDGNILGHKLEYNYNSHNVYIMAD | |
| | CGCGTCAATAATCTGGACGTTAAAGTCAAGA | | KQKNGIKVNFKIRHNIEDGSVQLADHYQQN | |
| | GCATAAATGGGGGATTCACCAGCTTTCTGAG | | TPIGDGPVLLPDNHYLSTQSALSKDPNEKR | |
| | GAGAAAGTGGAAGTTTAAGAAGGAACGAAAC | | DHMVLLEFVTAAGITLGMDELYK* | |
| | AAAGGATACAAGCACCATGCTGAGGATGCTT | | | |
| | TGATCATCGCTAACGCGGACTTTATCTTTAA | | | |
| | GGAATGGAAAAAGCTGGATAAGGCAAAGAAA | | | |
| | GTGATGGAAAACCAGATGTTCGAGGAGAAGC | | | |
| | AGGCAGAGTCAATGCCTGAGATCGAGACAGA | | | |
| | GCAGGAATACAAGGAAATTTTCATCACCCCT | | | |
| | CATCAGATTAAACACATAAAGGACTTCAAAG | | | |
| | ACTATAAATACTCTCATAGGGTGGACAAAAA | | | |
| | ACCCAATCGCAAGCTCATTAATGACACCCTG | | | |
| | TACTCAACACGGAAGGATGATAAAGGTAATA | | | |
| | CCTTGATTGTGAATAATCTTAATGGATTGTA | | | |
| | TGACAAAGATAACGACAAGCTCAAGAAGCTG | | | |
| | ATCAACAAGTCTCCAGAGAAGCTCCTTATGT | | | |
| | ATCACCACGACCCACAGACTTATCAGAAATT | | | |
| | GAAACTGATCATGGAGCAATACGGGGATGAG | | | |
| | AAGAACCCACTCTACAAATATTATGAGGAAA | | | |
| | CAGGTAATTACCTGACCAAGTACTCCAAGAA | | | |
| | GGATAACGGACCAGTGATCAAAAAGATAAAG | | | |
| | TACTATGGCAACAAACTTAATGCGCATTTGG | | | |
| | ACATAACTGACGATTACCCCAATTCTCGAAA | | | |
| | CAAGGTTGTGAAGCTCTCCCTGAAGCCTTAT | | | |
| | AGATTTGACGTGTACCTGGATAATGGGGTTT | | | |
| | ATAAATTCGTCACCGTGAAAAATCTGGACGT | | | |
| | GATCAAAAAGGAGAACTATTATGAAGTAAAC | | | |
| | TCAAAGTGCTATGAGGAGGCGAAGAAGCTGA | | | |
| | AGAAGATCTCCAATCAGGCCGAGTTCATCGC | | | |
| | TTCCTTCTATAAGAACGATCTCATCAAGATC | | | |
| | AATGGAGAGCTTTATCGCGTCATTGGTGTGA | | | |
| | ACAATGACTTGCTGAACAGGATCGAAGTCAA | | | |
| | TATGATAGACATTACCTACCGGGAGTATCTC | | | |
| | GAAAACATGAATGATAAACGGCCGCCTCACA | | | |
| | TCATCAAGACAATCGCATCTAAAACTCAGTC | | | |
| | AATAAAAAAGTACTCTACCGATATCCTGGGG | | | |
| | AATCTCTATGAAGTGAAGTCAAAGAAGCACC | | | |
| | CACAAATCATTAAAAAAGGTTCTGGAGGATC | | | |
| | TAGCGGAGGATCCTCTGGCAGCGAGACACCA | | | |
| | GGAACAAGCGAGTCAGCAACACCAGAGAGCA | | | |
| | GTGGCGGCAGCAGCGGCGGCAGCAGCACCCT | | | |
| | AAATATAGAAGATGAGTATCGGCTACATGAG | | | |
| | ACCTCAAAAGAGCCAGATGTTTCTCTAGGGT | | | |
| | CCACATGGCTGTCTGATTTTCCTCAGGCCTG | | | |
| | GGCGGAAACCGGGGGCATGGGACTGGCAGTT | | | |
| | CGCCAAGCTCCTCTGATCATACCTCTGAAAG | | | |
| | CAACCTCTACCCCCGTGTCCATAAAACAATA | | | |
| | CCCCATGTCACAAGAAGCCAGACTGGGGATC | | | |
| | AAGCCCCACATACAGAGACTGTTGGACCAGG | | | |
| | GAATACTGGTACCCTGCCAGTCCCCCTGGAA | | | |
| | CACGCCCCTGCTACCCGTTAAGAAACCAGGG | | | |
| | ACTAATGATTATAGGCCTGTCCAGGATCTGA | | | |
| | GAGAAGTCAACAAGCGGGTGGAAGACATCCA | | | |
| | CCCCACCGTGCCCAACCCTTACAACCTCTTG | | | |
| | AGCGGGCTCCCACCGTCCCACCAGTGGTACA | | | |
| | CTGTGCTTGATTTAAAGGATGCCTTTTTCTG | | | |
| | CCTGAGACTCCACCCCACCAGTCAGCCTCTC | | | |
| | TTCGCCTTTGAGTGGAGAGATCCAGAGATGG | | | |
| | GAATCTCAGGACAATTGACCTGGACCAGACT | | | |
| | CCCACAGGGTTTCAAAAACAGTCCCACCCTG | | | |
| | TTTAATGAGGCACTGCACAGAGACCTAGCAG | | | |
| | ACTTCCGGATCCAGCACCCAGACTTGATCCT | | | |
| | GCTACAGTACGTGGATGACTTACTGCTGGCC | | | |
| | GCCACTTCTGAGCTAGACTGCCAACAAGGTA | | | |
| | CTCGGGCCCTGTTACAAACCCTAGGGAACCT | | | |
| | CGGGTATCGGGCCTCGGCCAAGAAAGCCCAA | | | |
| | ATTTGCCAGAAACAGGTCAAGTATCTGGGGT | | | |
| | ATCTTCTAAAAGAGGGTCAGAGATGGCTGAC | | | |
| | TGAGGCCAGAAAAGAGACTGTGATGGGGCAG | | | |
| | CCTACTCCGAAGACCCCTCGACAACTAAGGG | | | |
| | AGTTCCTAGGGAAGGCAGGCTTCTGTCGCCT | | | |
| | CTTCATCCCTGGGTTTGCAGAAATGGCAGCC | | | |
| | CCCCTGTACCCTCTCACCAAACCGGGGACTC | | | |
| | TGTTTAATTGGGGCCCAGACCAACAAAAGGC | | | |
| | CTATCAAGAAATCAAGCAAGCTCTTCTAACT | | | |
| | GCCCCAGCCCTGGGGTTGCCAGATTTGACTA | | | |
| | AGCCCTTTGAACTCTTTGTCGACGAGAAGCA | | | |
| | GGGCTACGCCAAAGGTGTCCTAACGCAAAAA | | | |
| | CTGGGACCTTGGCGTCGGCCGGTGGCCTACC | | | |
| | TGTCCAAAAAGCTAGACCCAGTAGCAGCTGG | | | |
| | GTGGCCCCCTTGCCTACGGATGGTAGCAGCC | | | |
| | ATTGCCGTACTGACAAAGGATGCAGGCAAGC | | | |
| | TAACCATGGGACAGCCACTAGTCATTCTGGC | | | |
| | CCCCCATGCAGTAGAGGCACTAGTCAAACAA | | | |
| | CCCCCCGACCGCTGGCTTTCCAACGCCCGGA | | | |
| | TGACTCACTATCAGGCCTTGCTTTTGGACAC | | | |
| | GGACCGGGTCCAGTTCGGACCGGTGGTAGCC | | | |
| | CTGAACCCGGCTACGCTGCTCCCACTGCCTG | | | |
| | AGGAAGGGCTGCAACACAACTGCCTTGATAT | | | |
| | CCTGGCCGAAGCCCACGGAACCCGACCCGAC | | | |
| | CTAACGGACCAGCCGCTCCCAGACGCCGACC | | | |
| | ACACCTGGTACACGGATGGAAGCAGTCTCTT | | | |
| | ACAAGAGGGACAGCGTAAGGCGGGAGCTGCG | | | |
| | GTGACCACCGAGACCGAGGTAATCTGGGCTA | | | |
| | AAGCCCTGCCAGCCGGGACATCCGCTCAGCG | | | |
| | GGCTGAACTGATAGCACTCACCCAGGCCCTA | | | |
| | AAGATGGCAGAAGGTAAGAAGCTAAATGTTT | | | |
| | ATACTGATAGCCGTTATGCTTTTGCTACTGC | | | |
| | CCATATCCATGGAGAAATATACAGAAGGCGT | | | |
| | GGGTGGCTCACATCAGAAGGCAAAGAGATCA | | | |
| | AAAATAAAGACGAGATCTTGGCCCTACTAAA | | | |
| | AGCCCTCTTTCTGCCCAAAAGACTTAGCATA | | | |
| | ATCCATTGTCCAGGACATCAAAAGGGACACA | | | |
| | GCGCCGAGGCTAGAGGCAACCGGATGGCTGA | | | |
| | CCAAGCGGCCCGAAAGGCAGCCATCACAGAG | | | |
| | ACTCCAGACACCTCTACCCTCCTCATAGAAA | | | |
| | ATTCATCACCCTCTGGCGGCTCAAAAAGAAC | | | |
| | CGCCGACGGCAGCGAATTCGAGCCCAAGAAG | | | |
| | AAGAGGAAAGTCGGAAGCGGAGCTACTAACT | | | |
| | TCAGCCTGCTGAAGCAGGCTGGAGACGTGGA | | | |
| | GGAGAACCCTGGACCTATGGTGAGCAAGGGC | | | |
| | GAGGAGCTGTTCACCGGGGTGGTGCCCATCC | | | |
| | TGGTCGAGCTGGACGGCGACGTAAACGGCCA | | | |
| | CAAGTTCAGCGTGTCCGGCGAGGGCGAGGGC | | | |
| | GATGCCACCTACGGCAAGCTGACCCTGAAGT | | | |
| | TCATCTGCACCACCGGCAAGCTGCCCGTGCC | | | |
| | CTGGCCCACCCTCGTGACCACCCTGACCTAT | | | |
| | GGAGTGCAGTGCTTCAGCCGCTACCCCGACC | | | |
| | ACATGAAGCAGCACGACTTCTTCAAGTCCGC | | | |
| | CATGCCCGAAGGCTACGTCCAGGAGCGCACC | | | |
| | ATCTTCTTCAAGGACGACGGCAACTACAAGA | | | |
| | CCCGCGCCGAGGTGAAGTTCGAGGGCGACAC | | | |
| | CCTGGTGAACCGCATCGAGCTGAAGGGCATC | | | |
| | GACTTCAAGGAGGACGGCAACATCCTGGGGC | | | |
| | ACAAGCTGGAGTACAACTACAACAGCCACAA | | | |
| | CGTCTATATCATGGCCGACAAGCAGAAGAAC | | | |
| | GGCATCAAGGTGAACTTCAAGATCCGCCACA | | | |
| | ACATCGAGGACGGCAGCGTGCAGCTCGCCGA | | | |
| | CCACTACCAGCAGAACACCCCCATCGGCGAC | | | |
| | GGCCCCGTGCTGCTGCCCGACAACCACTACC | | | |
| | TGAGCACCCAGTCCGCCCTGAGCAAAGACCC | | | |
| | CAACGAGAAGCGCGATCACATGGTCCTGCTG | | | |
| | GAGTTCGTGACCGCCGCCGGGATCACTCTCG | | | |
| | GCATGGACGAGCTGTACAAGTAA | | | |
| bpNLS- | ATGAAACGGACAGCCGACGGAAGCGAGTTCG | 96 | MKRTADGSEFESPKKKRKVKRNYILGLDIG | |
| nSaCas9 | AGTCACCAAAGAAGAAGCGGAAAGTCAAACG | | ITSVGYGIIDYETRDVIDAGVRLFKEANVE | |
| (N580A) | GAACTACATCCTGGGGCTTGACATTGGGATA | | NNEGRRSKRGARRLKRRRRHRIQRVKKLLF | |
| KKH- | ACCAGCGTTGGCTACGGAATTATTGATTATG | | DYNLLTDHSELSGINPYEARVKGLSQKLSE | |
| XTEN- | AGACACGCGATGTGATTGACGCCGGGGTTAG | | EEFSAALLHLAKRRGVHNVNEVEEDTGNEL | |
| MMLVRT | GCTGTTCAAAGAGGCCAACGTTGAAAACAAC | | STKEQISRNSKALEEKYVAELQLERLKKDG | |
| (dRH)- | GAGGGAAGACGGAGTAAGCGCGGAGCAAGAA | | EVRGSINRFKTSDYVKEAKQLLKVQKAYHQ | |
| 4 AA | GACTCAAGCGCAGACGGAGACATCGGATTCA | | LDQSFIDTYIDLLETRRTYYEGPGEGSPFG | |
| linker- | GAGGGTGAAAAAGCTGCTCTTCGATTACAAT | | WKDIKEWYEMLMGHCTYFPEELRSVKYAYN | |
| bpNLS- | CTCCTGACCGATCATAGTGAGCTGAGCGGAA | | ADLYNALNDLNNLVITRDENEKLEYYEKFQ | |
| P2A- | TCAACCCCTACGAGGCGCGAGTGAAAGGGCT | | IIENVFKQKKKPTLKQIAKEILVNEEDIKG | |
| eGFP | TTCCCAGAAGCTGTCCGAAGAGGAGTTCTCC | | YRVTSTGKPEFTNLKVYHDIKDITARKEII | |
| | GCCGCGTTGCTGCACCTGGCCAAACGGAGGG | | ENAELLDQIAKILTIYQSSEDIQEELTNEN | |
| | GGGTTCACAATGTAAACGAAGTGGAGGAGGA | | SELTQEEIEQISNLKGYTGTHNLSLKAINL | |
| | CACGGGCAATGAACTTAGTACGAAAGAACAG | | ILDELWHINDNQIAIFNRLKLVPKKVDLSQ | |
| | ATCAGTAGGAACTCTAAGGCTCTCGAAGAGA | | QKEIPTTLVDDFILSPVVKRSFIQSIKVIN | |
| | AATACGTCGCTGAGTTGCAGCTTGAGAGACT | | AIIKKYGLPNDIIIELAREKNSKDAQKMIN | |
| | GAAAAAAGACGGCGAAGTACGCGGATCTATT | | EMQKRNRQTNERIEEIIRTTGKENAKYLIE | |
| | AATAGGTTCAAGACTTCAGATTACGTAAAGG | | KIKLHDMQEGKCLYSLEAIPLEDLLNNPEN | |
| | AAGCCAAGCAGCTCCTGAAAGTACAGAAAGC | | YEVDHIIPRSVSFDNSFNNKVLVKQEEASK | |
| | GTACCATCAGCTCGATCAGAGCTTCATCGAT | | KGNRTPFQYLSSSDSKISYETFKKHIUNLA | |
| | ACCTACATAGATTTGCTGGAGACACGGAGGA | | KGKGRISKTKKEYLLEERDINRESVQKDFI | |
| | CATACTACGAGGGCCCAGGGGAAGGATCTCC | | NRNLVDTRYATRGLMNLLRSYFRVNNLDVK | |
| | TTTTGGGTGGAAGGACATCAAGGAATGGTAC | | VKSINGGFTSFLRRKWKFKKERNKGYKHHA | |
| | GAGATGCTTATGGGACATTGTACATATTTTC | | EDALIIANADFIFKEWKKLDKAKKVMENQM | |
| | CGGAGGAGCTCAGGAGCGTCAAGTACGCCTA | | FEEKQAESMPEIETEQEYKEIFITPHQIKH | |
| | CAATGCCGACCTGTACAATGCCCTCAATGAC | | IKDFKDYKYSHRVDKKPNRKLINDTLYSTR | |
| | CTCAATAACCTCGTGATTACCAGGGACGAGA | | KDDKGNTLIVNNLNGLYDKDNDKLKKLINK | |
| | ACGAGAAGCTGGAGTACTATGAAAAGTTCCA | | SPEKLLMYHHDPQTYQKLKLIMEQYGDEKN | |
| | GATTATCGAGAATGTGTTTAAGCAGAAGAAG | | PLYKYYEETGNYLTKYSKKDNGPVIKKIKY | |
| | AAGCCGACACTTAAGCAGATTGCAAAGGAAA | | YGNKLNAHLDITDDYPNSRNKVVKLSLKPY | |
| | TCCTCGTGAATGAGGAAGATATCAAGGGATA | | RFDVYLDNGVYKFVTVKNLDVIKKENYYEV | |
| | CAGAGTGACAAGTACAGGCAAGCCCGAGTTC | | NSKCYEEAKKLKKISNQAEFIASFYKNDLI | |
| | ACAAATCTGAAGGTGTACCACGATATTAAGG | | KINGELYRVIGVNNDLLNRIEVNMIDITYR | |
| | ACATAACCGCACGAAAGGAGATAATCGAAAA | | EYLENMNDKRPPHIIKTIASKTQSIKKYST | |
| | CGCTGAGCTCCTCGATCAGATCGCAAAAATT | | DILGNLYEVKSKKHPQIIKKGSGGSSGGSS | |
| | CTTACCATCTACCAGTCTAGTGAGGACATTC | | GSETPGTSESATPESSGGSSGGSSTLNIED | |
| | AGGAGGAACTGACTAATCTGAACAGTGAGCT | | EYRLHETSKEPDVSLGSTWLSDFPQAWAET | |
| | CACCCAAGAGGAAATTGAGCAGATTTCAAAC | | GGMGLAVRQAPLIIPLKATSTPVSIKQYPM | |
| | CTGAAAGGCTACACCGGGACGCACAATCTGA | | SQEARLGIKPHIQRLLDQGILVPCQSPWNT | |
| | GCCTCAAAGCAATCAACCTCATTCTGGATGA | | PLLPVKKPGTNDYRPVQDLREVNERVEDIH | |
| | ACTTTGGCACACAAATGACAACCAAATTGCC | | PTVPNPYNLLSGLPPSHQWYTVLDLKDAFF | |
| | ATATTCAACCGCCTGAAACTGCTGCCAAAAA | | CLRLHPTSQPLFAFEWRDPEMGISGQUTWT | |
| | AAGTGGATCTGTCACAGCAAAAGGAAATCCC | | RLPQGFKNSPTLFNEALHRDLADFRIQHPD | |
| | TACAACCTTGGTTGACGATTTTATTCTGTCC | | LILLQYVDDLLLAATSELDCQQGTRALLQT | |
| | CCCGTTGTCAAGCGGAGCTTCATCCAGTCAA | | LGNLGYRASAKKAQICQKQVKYLGYLLKEG | |
| | TCAAGGTGATCAATGCCATCATTAAAAAATA | | QRWLTEARKETVMGQPTPKTPRQLREFLGK | |
| | CGGATTGCCAAACGATATAATTATCGAGCTT | | AGFCRLFIPGFAEMAAPLYPLTKPGTLENW | |
| | GCACGAGAGAAGAACTCAAAGGACGCCCAGA | | GPDQQKAYQEIKQALLTAPALGLPDLIKPE | |
| | AGATGATTAACGAAATGCAGAAGCGCAACCG | | ELFVDEKQGYAKGVLTQKLGPWRRPVAYLS | |
| | CCAGACAAACGAACGCATAGAGGAAATTATA | | KKLDPVAAGWPPCLRMVAAIAVLTKDAGKL | |
| | AGAACAACCGGCAAAGAGAATGCCAAGTATC | | TMGQPLVILAPHAVEALVKQPPDRWLSNAR | |
| | TGATCGAGAAAATCAAGCTGCACGACATGCA | | MTHYQALLLDTDRVQFGPVVALNPATLLPL | |
| | AGAAGGCAAGTGCCTGTACTCTCTGGAAGCT | | PEEGLQHNCLSGGSKRTADGSEFEPKKKRK | |
| | ATCCCACTCGAAGATCTGCTGAATAATCCAT | | VGSGATNFSLLKQAGDVEENPGPMVSKGEE | |
| | TCAATTACGAGGTGGACCACATCATCCCTAG | | LFTGVVPILVELDGDVNGHKFSVSGEGEGD | |
| | ATCCGTAAGCTTTGACAATTCCTTCAATAAC | | ATYGKLTLKFICTTGKLPVPWPTLVTILTY | |
| | AAAGTTCTGGTTAAACAGGAGGAAGCCTCTA | | GVQCFSRYPDHMKQHDFFKSAMPEGYVQER | |
| | AAAAAGGGAACCGGACCCCGTTCCAGTACCT | | TIFFKDDGNYKTRAEVKFEGDTLVNRIELK | |
| | GAGCTCCAGTGACAGCAAGATTAGCTACGAG | | GIDFKEDGNILGHKLEYNYNSHNVYIMADK | |
| | ACTTTTAAGAAACATATTCTGAATCTGGCCA | | QKNGIKVNFKIRHNIEDGSVQLADHYQQNT | |
| | AAGGCAAAGGCAGGATCAGCAAGACCAAGAA | | PIGDGPVLLPDNHYLSTQSALSKDPNEKRD | |
| | GGAGTACCTCCTCGAAGAACGCGACATTAAC | | HMVLLEFVTAAGITLGMDELYK* | |
| | AGATTTAGTGTGCAGAAAGATTTCATCAACC | | | |
| | GAAACCTTGTCGATACTCGGTACGCCACGAG | | | |
| | AGGCCTGATGAATCTCCTCAGGAGCTACTTC | | | |
| | CGCGTCAATAATCTGGACGTTAAAGTCAAGA | | | |
| | GCATAAATGGGGGATTCACCAGCTTTCTGAG | | | |
| | GAGAAAGTGGAAGTTTAAGAAGGAACGAAAC | | | |
| | AAAGGATACAAGCACCATGCTGAGGATGCTT | | | |
| | TGATCATCGCTAACGCGGACTTTATCTTTAA | | | |
| | GGAATGGAAAAAGCTGGATAAGGCAAAGAAA | | | |
| | GTGATGGAAAACCAGATGTTCGAGGAGAAGC | | | |
| | AGGCAGAGTCAATGCCTGAGATCGAGACAGA | | | |
| | GCAGGAATACAAGGAAATTTTCATCACCCCT | | | |
| | CATCAGATTAAACACATAAAGGACTTCAAAG | | | |
| | ACTATAAATACTCTCATAGGGTGGACAAAAA | | | |
| | ACCCAATCGCAAGCTCATTAATGACACCCTG | | | |
| | TACTCAACACGGAAGGATGATAAAGGTAATA | | | |
| | CCTTGATTGTGAATAATCTTAATGGATTGTA | | | |
| | TGACAAAGATAACGACAAGCTCAAGAAGCTG | | | |
| | ATCAACAAGTCTCCAGAGAAGCTCCTTATGT | | | |
| | ATCACCACGACCCACAGACTTATCAGAAATT | | | |
| | GAAACTGATCATGGAGCAATACGGGGATGAG | | | |
| | AAGAACCCACTCTACAAATATTATGAGGAAA | | | |
| | CAGGTAATTACCTGACCAAGTACTCCAAGAA | | | |
| | GGATAACGGACCAGTGATCAAAAAGATAAAG | | | |
| | TACTATGGCAACAAACTTAATGCGCATTTGG | | | |
| | ACATAACTGACGATTACCCCAATTCTCGAAA | | | |
| | CAAGGTTGTGAAGCTCTCCCTGAAGCCTTAT | | | |
| | AGATTTGACGTGTACCTGGATAATGGGGTTT | | | |
| | ATAAATTCGTCACCGTGAAAAATCTGGACGT | | | |
| | GATCAAAAAGGAGAACTATTATGAAGTAAAC | | | |
| | TCAAAGTGCTATGAGGAGGCGAAGAAGCTGA | | | |
| | AGAAGATCTCCAATCAGGCCGAGTTCATCGC | | | |
| | TTCCTTCTATAAGAACGATCTCATCAAGATC | | | |
| | AATGGAGAGCTTTATCGCGTCATTGGTGTGA | | | |
| | ACAATGACTTGCTGAACAGGATCGAAGTCAA | | | |
| | TATGATAGACATTACCTACCGGGAGTATCTC | | | |
| | GAAAACATGAATGATAAACGGCCGCCTCACA | | | |
| | TCATCAAGACAATCGCATCTAAAACTCAGTC | | | |
| | AATAAAAAAGTACTCTACCGATATCCTGGGG | | | |
| | AATCTCTATGAAGTGAAGTCAAAGAAGCACC | | | |
| | CACAAATCATTAAAAAAGGTTCTGGAGGATC | | | |
| | TAGCGGAGGATCCTCTGGCAGCGAGACACCA | | | |
| | GGAACAAGCGAGTCAGCAACACCAGAGAGCA | | | |
| | GTGGCGGCAGCAGCGGCGGCAGCAGCACCCT | | | |
| | AAATATAGAAGATGAGTATCGGCTACATGAG | | | |
| | ACCTCAAAAGAGCCAGATGTTTCTCTAGGGT | | | |
| | CCACATGGCTGTCTGATTTTCCTCAGGCCTG | | | |
| | GGCGGAAACCGGGGGCATGGGACTGGCAGTT | | | |
| | CGCCAAGCTCCTCTGATCATACCTCTGAAAG | | | |
| | CAACCTCTACCCCCGTGTCCATAAAACAATA | | | |
| | CCCCATGTCACAAGAAGCCAGACTGGGGATC | | | |
| | AAGCCCCACATACAGAGACTGTTGGACCAGG | | | |
| | GAATACTGGTACCCTGCCAGTCCCCCTGGAA | | | |
| | CACGCCCCTGCTACCCGTTAAGAAACCAGGG | | | |
| | ACTAATGATTATAGGCCTGTCCAGGATCTGA | | | |
| | GAGAAGTCAACAAGCGGGTGGAAGACATCCA | | | |
| | CCCCACCGTGCCCAACCCTTACAACCTCTTG | | | |
| | AGCGGGCTCCCACCGTCCCACCAGTGGTACA | | | |
| | CTGTGCTTGATTTAAAGGATGCCTTTTTCTG | | | |
| | CCTGAGACTCCACCCCACCAGTCAGCCTCTC | | | |
| | TTCGCCTTTGAGTGGAGAGATCCAGAGATGG | | | |
| | GAATCTCAGGACAATTGACCTGGACCAGACT | | | |
| | CCCACAGGGTTTCAAAAACAGTCCCACCCTG | | | |
| | TTTAATGAGGCACTGCACAGAGACCTAGCAG | | | |
| | ACTTCCGGATCCAGCACCCAGACTTGATCCT | | | |
| | GCTACAGTACGTGGATGACTTACTGCTGGCC | | | |
| | GCCACTTCTGAGCTAGACTGCCAACAAGGTA | | | |
| | CTCGGGCCCTGTTACAAACCCTAGGGAACCT | | | |
| | CGGGTATCGGGCCTCGGCCAAGAAAGCCCAA | | | |
| | ATTTGCCAGAAACAGGTCAAGTATCTGGGGT | | | |
| | ATCTTCTAAAAGAGGGTCAGAGATGGCTGAC | | | |
| | TGAGGCCAGAAAAGAGACTGTGATGGGGCAG | | | |
| | CCTACTCCGAAGACCCCTCGACAACTAAGGG | | | |
| | AGTTCCTAGGGAAGGCAGGCTTCTGTCGCCT | | | |
| | CTTCATCCCTGGGTTTGCAGAAATGGCAGCC | | | |
| | CCCCTGTACCCTCTCACCAAACCGGGGACTC | | | |
| | TGTTTAATTGGGGCCCAGACCAACAAAAGGC | | | |
| | CTATCAAGAAATCAAGCAAGCTCTTCTAACT | | | |
| | GCCCCAGCCCTGGGGTTGCCAGATTTGACTA | | | |
| | AGCCCTTTGAACTCTTTGTCGACGAGAAGCA | | | |
| | GGGCTACGCCAAAGGTGTCCTAACGCAAAAA | | | |
| | CTGGGACCTTGGCGTCGGCCGGTGGCCTACC | | | |
| | TGTCCAAAAAGCTAGACCCAGTAGCAGCTGG | | | |
| | GTGGCCCCCTTGCCTACGGATGGTAGCAGCC | | | |
| | ATTGCCGTACTGACAAAGGATGCAGGCAAGC | | | |
| | TAACCATGGGACAGCCACTAGTCATTCTGGC | | | |
| | CCCCCATGCAGTAGAGGCACTAGTCAAACAA | | | |
| | CCCCCCGACCGCTGGCTTTCCAACGCCCGGA | | | |
| | TGACTCACTATCAGGCCTTGCTTTTGGACAC | | | |
| | GGACCGGGTCCAGTTCGGACCGGTGGTAGCC | | | |
| | CTGAACCCGGCTACGCTGCTCCCACTGCCTG | | | |
| | AGGAAGGGCTGCAACACAACTGCCTTTCTGG | | | |
| | CGGCTCAAAAAGAACCGCCGACGGCAGCGAA | | | |
| | TTCGAGCCCAAGAAGAAGAGGAAAGTCGGAA | | | |
| | GCGGAGCTACTAACTTCAGCCTGCTGAAGCA | | | |
| | GGCTGGAGACGTGGAGGAGAACCCTGGACCT | | | |
| | ATGGTGAGCAAGGGCGAGGAGCTGTTCACCG | | | |
| | GGGTGGTGCCCATCCTGGTCGAGCTGGACGG | | | |
| | CGACGTAAACGGCCACAAGTTCAGCGTGTCC | | | |
| | GGCGAGGGCGAGGGCGATGCCACCTACGGCA | | | |
| | AGCTGACCCTGAAGTTCATCTGCACCACCGG | | | |
| | CAAGCTGCCCGTGCCCTGGCCCACCCTCGTG | | | |
| | ACCACCCTGACCTATGGAGTGCAGTGCTTCA | | | |
| | GCCGCTACCCCGACCACATGAAGCAGCACGA | | | |
| | CTTCTTCAAGTCCGCCATGCCCGAAGGCTAC | | | |
| | GTCCAGGAGCGCACCATCTTCTTCAAGGACG | | | |
| | ACGGCAACTACAAGACCCGCGCCGAGGTGAA | | | |
| | GTTCGAGGGCGACACCCTGGTGAACCGCATC | | | |
| | GAGCTGAAGGGCATCGACTTCAAGGAGGACG | | | |
| | GCAACATCCTGGGGCACAAGCTGGAGTACAA | | | |
| | CTACAACAGCCACAACGTCTATATCATGGCC | | | |
| | GACAAGCAGAAGAACGGCATCAAGGTGAACT | | | |
| | TCAAGATCCGCCACAACATCGAGGACGGCAG | | | |
| | CGTGCAGCTCGCCGACCACTACCAGCAGAAC | | | |
| | ACCCCCATCGGCGACGGCCCCGTGCTGCTGC | | | |
| | CCGACAACCACTACCTGAGCACCCAGTCCGC | | | |
| | CCTGAGCAAAGACCCCAACGAGAAGCGCGAT | | | |
| | CACATGGTCCTGCTGGAGTTCGTGACCGCCG | | | |
| | CCGGGATCACTCTCGGCATGGACGAGCTGTA | | | |
| | CAAGTAA | | | |
| bpNLS | ATGAAACGGACAGCCGACGGAAGCGAGTTCG | 98 | MKRTADGSEFESPKKKRKVKRNYILGLDIG | 99 |
| -nSaCas9 | AGTCACCAAAGAAGAAGCGGAAAGTCAAACG | | ITSVGYGIIDYETRDVIDAGVRLFKEANVE | |
| (N580A) | GAACTACATCCTGGGGCTTGACATTGGGATA | | NNEGRRSKRGARRLKRRRRHRIQRVKKLLE | |
| KKH-XTEN- | ACCAGCGTTGGCTACGGAATTATTGATTATG | | DYNLLIDHSELSGINPYEARVKGLSQKLSE | |
| 4 AA | AGACACGCGATGTGATTGACGCCGGGGTTAG | | EEFSAALLHLAKRRGVHNVNEVEEDTGNEL | |
| linker- | GCTGTTCAAAGAGGCCAACGTTGAAAACAAC | | STKEQISRNSKALEEKYVAELQLERLKKDG | |
| bpNLS- | GAGGGAAGACGGAGTAAGCGCGGAGCAAGAA | | EVRGSINRFKTSDYVKEAKQLLKVQKAYHQ | |
| P2A-eGFP | GACTCAAGCGCAGACGGAGACATCGGATTCA | | LDQSFIDTYIDLLETRRTYYEGPGEGSPFG | |
| | GAGGGTGAAAAAGCTGCTCTTCGATTACAAT | | WKDIKEWYEMLMGHCTYFPEELRSVKYAYN | |
| | CTCCTGACCGATCATAGTGAGCTGAGCGGAA | | ADLYNALNDLNNLVITRDENEKLEYYEKFQ | |
| | TCAACCCCTACGAGGCGCGAGTGAAAGGGCT | | IIENVFKQKKKPTLKQIAKEILVNEEDIKG | |
| | TTCCCAGAAGCTGTCCGAAGAGGAGTTCTCC | | YRVTSTGKPEFTNLKVYHDIKDITARKEII | |
| | GCCGCGTTGCTGCACCTGGCCAAACGGAGGG | | ENAELLDQIAKILTIYQSSEDIQEELTNEN | |
| | GGGTTCACAATGTAAACGAAGTGGAGGAGGA | | SELTQEEIEQISNLKGYTGTHNLSLKAINL | |
| | CACGGGCAATGAACTTAGTACGAAAGAACAG | | ILDELWHINDNQIAIFNRLKLVPKKVDLSQ | |
| | ATCAGTAGGAACTCTAAGGCTCTCGAAGAGA | | QKEIPTTLVDDFILSPVVKRSFIQSIKVIN | |
| | AATACGTCGCTGAGTTGCAGCTTGAGAGACT | | AIIKKYGLENDIIIELAREKNSKDAQKMIN | |
| | GAAAAAAGACGGCGAAGTACGCGGATCTATT | | EMQKRNRQTNERIEEIIRTTGKENAKYLIE | |
| | AATAGGTTCAAGACTTCAGATTACGTAAAGG | | KIKLHDMQEGKCLYSLEAIPLEDLINNPEN | |
| | AAGCCAAGCAGCTCCTGAAAGTACAGAAAGC | | YEVDHIIPRSVSFDNSFNNKVLVKQEEASK | |
| | GTACCATCAGCTCGATCAGAGCTTCATCGAT | | KGNRTPFQYLSSSDSKISYETFKKHILNLA | |
| | ACCTACATAGATTTGCTGGAGACACGGAGGA | | KGKGRISKTKKEYLLEERDINRFSVQKDEI | |
| | CATACTACGAGGGCCCAGGGGAAGGATCTCC | | NRNLVDTRYATRGLMNLLRSYFRVNNLDVK | |
| | TTTTGGGTGGAAGGACATCAAGGAATGGTAC | | VKSINGGFTSFLRRKWKFKKERNKGYKHHA | |
| | GAGATGCTTATGGGACATTGTACATATTTTC | | EDALIIANADFIFKEWKKLDKAKKVMENQM | |
| | CGGAGGAGCTCAGGAGCGTCAAGTACGCCTA | | PEEKQAESMPEIETEQEYKEIFITPHQIKH | |
| | CAATGCCGACCTGTACAATGCCCTCAATGAC | | IKDFKDYKYSHRVDKKPNRKLINDTLYSTR | |
| | CTCAATAACCTCGTGATTACCAGGGACGAGA | | KDDKGNTLIVNNLNGLYDKDNDKLKKLINK | |
| | ACGAGAAGCTGGAGTACTATGAAAAGTTCCA | | SPEKLLMYHHDPQTYQKLKLIMEQYGDEKN | |
| | GATTATCGAGAATGTGTTTAAGCAGAAGAAG | | PLYKYYEETGNYLTKYSKKDNGPVIKKIKY | |
| | AAGCCGACACTTAAGCAGATTGCAAAGGAAA | | YGNKLNAHLDITDDYPNSRNKVVKLSLKPY | |
| | TCCTCGTGAATGAGGAAGATATCAAGGGATA | | RFDVYLDNGVYKFVTVKNLDVIKKENYYEV | |
| | CAGAGTGACAAGTACAGGCAAGCCCGAGTTC | | NSKCYEEAKKLKKISNQAEFIASFYKNDEI | |
| | ACAAATCTGAAGGTGTACCACGATATTAAGG | | KINGELYRVIGVNNDLLNRIEVNMIDITYR | |
| | ACATAACCGCACGAAAGGAGATAATCGAAAA | | EYLENMNDKRPPHIIKTIASKTQSIKKYST | |
| | CGCTGAGCTCCTCGATCAGATCGCAAAAATT | | DILQNLYEVKSKKHPQIIKKGSGGSSGGSS | |
| | CTTACCATCTACCAGTCTAGTGAGGACATTC | | GSETPGTSESATPESSGGSSGGSSSGGSKR | |
| | AGGAGGAACTGACTAATCTGAACAGTGAGCT | | TADGSEFEPKKKRKVGSGATNFSLLKQAGD | |
| | CACCCAAGAGGAAATTGAGCAGATTTCAAAC | | VEENPGPMVSKGEELFTGVVPILVELDGDV | |
| | CTGAAAGGCTACACCGGGACGCACAATCTGA | | NGHKFSVSGEGEGDATYGKLILKFICTTGK | |
| | GCCTCAAAGCAATCAACCTCATTCTGGATGA | | LPVPWPTLVTTLTYGVQCFSRYPDHMKQHD | |
| | ACTTTGGCACACAAATGACAACCAAATTGCC | | FFKSAMPEGYVQERTIFFKDDGNYKTRAEV | |
| | ATATTCAACCGCCTGAAACTGCTGCCAAAAA | | KPEGDTLVNRIELKGIDFKEDGNILGHKLE | |
| | AAGTGGATCTGTCACAGCAAAAGGAAATCCC | | YNYNSHNVYIMADKQKNGIKVNFKIRHNIE | |
| | TACAACCTTGGTTGACGATTTTATTCTGTCC | | DGSVQLADHYQQNTPIGDGPVLLPDNHYLS | |
| | CCCGTTGTCAAGCGGAGCTTCATCCAGTCAA | | TQSALSKDPNEKRDHMVLLEFVTAAGITLG | |
| | TCAAGGTGATCAATGCCATCATTAAAAAATA | | MDELYK* | |
| | CGGATTGCCAAACGATATAATTATCGAGCTT | | | |
| | GCACGAGAGAAGAACTCAAAGGACGCCCAGA | | | |
| | AGATGATTAACGAAATGCAGAAGCGCAACCG | | | |
| | CCAGACAAACGAACGCATAGAGGAAATTATA | | | |
| | AGAACAACCGGCAAAGAGAATGCCAAGTATC | | | |
| | TGATCGAGAAAATCAAGCTGCACGACATGCA | | | |
| | AGAAGGCAAGTGCCTGTACTCTCTGGAAGCT | | | |
| | ATCCCACTCGAAGATCTGCTGAATAATCCAT | | | |
| | TCAATTACGAGGTGGACCACATCATCCCTAG | | | |
| | ATCCGTAAGCTTTGACAATTCCTTCAATAAC | | | |
| | AAAGTTCTGGTTAAACAGGAGGAAGCCTCTA | | | |
| | AAAAAGGGAACCGGACCCCGTTCCAGTACCT | | | |
| | GAGCTCCAGTGACAGCAAGATTAGCTACGAG | | | |
| | ACTTTTAAGAAACATATTCTGAATCTGGCCA | | | |
| | AAGGCAAAGGCAGGATCAGCAAGACCAAGAA | | | |
| | GGAGTACCTCCTCGAAGAACGCGACATTAAC | | | |
| | AGATTTAGTGTGCAGAAAGATTTCATCAACC | | | |
| | GAAACCTTGTCGATACTCGGTACGCCACGAG | | | |
| | AGGCCTGATGAATCTCCTCAGGAGCTACTTC | | | |
| | CGCGTCAATAATCTGGACGTTAAAGTCAAGA | | | |
| | GCATAAATGGGGGATTCACCAGCTTTCTGAG | | | |
| | GAGAAAGTGGAAGTTTAAGAAGGAACGAAAC | | | |
| | AAAGGATACAAGCACCATGCTGAGGATGCTT | | | |
| | TGATCATCGCTAACGCGGACTTTATCTTTAA | | | |
| | GGAATGGAAAAAGCTGGATAAGGCAAAGAAA | | | |
| | GTGATGGAAAACCAGATGTTCGAGGAGAAGC | | | |
| | AGGCAGAGTCAATGCCTGAGATCGAGACAGA | | | |
| | GCAGGAATACAAGGAAATTTTCATCACCCCT | | | |
| | CATCAGATTAAACACATAAAGGACTTCAAAG | | | |
| | ACTATAAATACTCTCATAGGGTGGACAAAAA | | | |
| | ACCCAATCGCAAGCTCATTAATGACACCCTG | | | |
| | TACTCAACACGGAAGGATGATAAAGGTAATA | | | |
| | CCTTGATTGTGAATAATCTTAATGGATTGTA | | | |
| | TGACAAAGATAACGACAAGCTCAAGAAGCTG | | | |
| | ATCAACAAGTCTCCAGAGAAGCTCCTTATGT | | | |
| | ATCACCACGACCCACAGACTTATCAGAAATT | | | |
| | GAAACTGATCATGGAGCAATACGGGGATGAG | | | |
| | AAGAACCCACTCTACAAATATTATGAGGAAA | | | |
| | CAGGTAATTACCTGACCAAGTACTCCAAGAA | | | |
| | GGATAACGGACCAGTGATCAAAAAGATAAAG | | | |
| | TACTATGGCAACAAACTTAATGCGCATTTGG | | | |
| | ACATAACTGACGATTACCCCAATTCTCGAAA | | | |
| | CAAGGTTGTGAAGCTCTCCCTGAAGCCTTAT | | | |
| | AGATTTGACGTGTACCTGGATAATGGGGTTT | | | |
| | ATAAATTCGTCACCGTGAAAAATCTGGACGT | | | |
| | GATCAAAAAGGAGAACTATTATGAAGTAAAC | | | |
| | TCAAAGTGCTATGAGGAGGCGAAGAAGCTGA | | | |
| | AGAAGATCTCCAATCAGGCCGAGTTCATCGC | | | |
| | TTCCTTCTATAAGAACGATCTCATCAAGATC | | | |
| | AATGGAGAGCTTTATCGCGTCATTGGTGTGA | | | |
| | ACAATGACTTGCTGAACAGGATCGAAGTCAA | | | |
| | TATGATAGACATTACCTACCGGGAGTATCTC | | | |
| | GAAAACATGAATGATAAACGGCCGCCTCACA | | | |
| | TCATCAAGACAATCGCATCTAAAACTCAGTC | | | |
| | AATAAAAAAGTACTCTACCGATATCCTGGGG | | | |
| | AATCTCTATGAAGTGAAGTCAAAGAAGCACC | | | |
| | CACAAATCATTAAAAAAGGTTCTGGAGGATC | | | |
| | TAGCGGAGGATCCTCTGGCAGCGAGACACCA | | | |
| | GGAACAAGCGAGTCAGCAACACCAGAGAGCA | | | |
| | GTGGCGGCAGCAGCGGCGGCAGCAGCTCTGG | | | |
| | CGGCTCAAAAAGAACCGCCGACGGCAGCGAA | | | |
| | TTCGAGCCCAAGAAGAAGAGGAAAGTCGGAA | | | |
| | GCGGAGCTACTAACTTCAGCCTGCTGAAGCA | | | |
| | GGCTGGAGACGTGGAGGAGAACCCTGGACCT | | | |
| | ATGGTGAGCAAGGGCGAGGAGCTGTTCACCG | | | |
| | GGGTGGTGCCCATCCTGGTCGAGCTGGACGG | | | |
| | CGACGTAAACGGCCACAAGTTCAGCGTGTCC | | | |
| | GGCGAGGGCGAGGGCGATGCCACCTACGGCA | | | |
| | AGCTGACCCTGAAGTTCATCTGCACCACCGG | | | |
| | CAAGCTGCCCGTGCCCTGGCCCACCCTCGTG | | | |
| | ACCACCCTGACCTATGGAGTGCAGTGCTTCA | | | |
| | GCCGCTACCCCGACCACATGAAGCAGCACGA | | | |
| | CTTCTTCAAGTCCGCCATGCCCGAAGGCTAC | | | |
| | GTCCAGGAGCGCACCATCTTCTTCAAGGACG | | | |
| | ACGGCAACTACAAGACCCGCGCCGAGGTGAA | | | |
| | GTTCGAGGGCGACACCCTGGTGAACCGCATC | | | |
| | GAGCTGAAGGGCATCGACTTCAAGGAGGACG | | | |
| | GCAACATCCTGGGGCACAAGCTGGAGTACAA | | | |
| | CTACAACAGCCACAACGTCTATATCATGGCC | | | |
| | GACAAGCAGAAGAACGGCATCAAGGTGAACT | | | |
| | TCAAGATCCGCCACAACATCGAGGACGGCAG | | | |
| | CGTGCAGCTCGCCGACCACTACCAGCAGAAC | | | |
| | ACCCCCATCGGCGACGGCCCCGTGCTGCTGC | | | |
| | CCGACAACCACTACCTGAGCACCCAGTCCGC | | | |
| | CCTGAGCAAAGACCCCAACGAGAAGCGCGAT | | | |
| | CACATGGTCCTGCTGGAGTTCGTGACCGCCG | | | |
| | CCGGGATCACTCTCGGCATGGACGAGCTGTA | | | |
| | CAAGTAA | | | |
| bpNLS- | ATGAAACGGACAGCCGACGGAAGCGAGTTCG | 100 | MKRTADGSEFESPKKKRKVKRNYILGLDIG | 101 |
| nSaCas9 | AGTCACCAAAGAAGAAGCGGAAAGTCAAGCG | | ITSVGYGIIDYETRDVIDAGVRLFKEANVE | |
| (N580A) | GAACTACATCCTGGGCCTGGACATCGGCATC | | NNEGRRSKRGARRLKRRRRHRIQRVKKLLF | |
| -XTEN- | ACCAGCGTGGGCTACGGCATCATCGACTACG | | DYNLLIDHSELSGINPYEARVKGLSQKLSE | |
| 4 AA | AGACACGGGACGTGATCGATGCCGGCGTGCG | | EEFSAALLHLAKRRGVHNVNEVEEDTGNEL | |
| linker- | GCTGTTCAAAGAGGCCAACGTGGAAAACAAC | | STKEQISRNSKALEEKYVAELQLERLKKDG | |
| bpNLS- | GAGGGCAGGCGGAGCAAGAGAGGCGCCAGAA | | EVRGSINRFKTSDYVKEAKQLLKVQKAYHQ | |
| P2A-eGFP | GGCTGAAGCGGCGGAGGCGGCATAGAATCCA | | LDQSFIDTYIDLLETRRTYYEGPGEGSPFG | |
| | GAGAGTGAAGAAGCTGCTGTTCGACTACAAC | | WKDIKEWYEMLMGHCTYFPEELRSVKYAYN | |
| | CTGCTGACCGACCACAGCGAGCTGAGCGGCA | | ADLYNALNDLNNLVITRDENEKLEYYEKFQ | |
| | TCAACCCCTACGAGGCCAGAGTGAAGGGCCT | | IIENVFKQKKKPTLKQIAKEILVNEEDIKG | |
| | GAGCCAGAAGCTGAGCGAGGAAGAGTTCTCT | | YRVTSTGKPEFTNLKVYHDIKDITARKEII | |
| | GCCGCCCTGCTGCACCTGGCCAAGAGAAGAG | | ENAELLDQIAKILTIYQSSEDIQEELINEN | |
| | GCGTGCACAACGTGAACGAGGTGGAAGAGGA | | SELTQEEIEQISNLKGYTGTHNLSLKAINL | |
| | CACCGGCAACGAGCTGTCCACCAAAGAGCAG | | ILDELWHINDNQIAIFNRLKLVPKKVDLSQ | |
| | ATCAGCCGGAACAGCAAGGCCCTGGAAGAGA | | QKEIPTTLVDDFILSPVVKRSFIQSIKVIN | |
| | AATACGTGGCCGAACTGCAGCTGGAACGGCT | | AIIKKYGLENDIIIELAREKNSKDAQKMIN | |
| | GAAGAAAGACGGCGAAGTGCGGGGCAGCATC | | EMQKRNRQTNERIEEIIRTIGKENAKYLIE | |
| | AACAGATTCAAGACCAGCGACTACGTGAAAG | | KIKLHDMQEGKCLYSLEAIPLEDLINNPEN | |
| | AAGCCAAACAGCTGCTGAAGGTGCAGAAGGC | | YEVDHIIPRSVSFDNSFNNKVLVKQEEASK | |
| | CTACCACCAGCTGGACCAGAGCTTCATCGAC | | KGNRTPFQYLSSSDSKISYETFKKHILNLA | |
| | ACCTACATCGACCTGCTGGAAACCCGGCGGA | | KGKGRISKTKKEYLLEERDINRFSVQKDFI | |
| | CCTACTATGAGGGACCTGGCGAGGGCAGCCC | | NRNLVDTRYATRGLMNLLRSYFRVNNLDVK | |
| | CTTCGGCTGGAAGGACATCAAAGAATGGTAC | | VKSINGGFTSFLRRKWKFKKERNKGYKHHA | |
| | GAGATGCTGATGGGCCACTGCACCTACTTCC | | EDALIIANADFIFKEWKKLDKAKKVMENQM | |
| | CCGAGGAACTGCGGAGCGTGAAGTACGCCTA | | FEEKQAESMPEIETEQEYKEIFITPHQIKH | |
| | CAACGCCGACCTGTACAACGCCCTGAACGAC | | IKDFKDYKYSHRVDKKPNRELINDTLYSTR | |
| | CTGAACAATCTCGTGATCACCAGGGACGAGA | | KDDKGNTLIVNNINGLYDKDNDKLKKLINK | |
| | ACGAGAAGCTGGAATATTACGAGAAGTTCCA | | SPEKLLMYHHDPQTYQKLKLIMEQYGDEKN | |
| | GATCATCGAGAACGTGTTCAAGCAGAAGAAG | | PLYKYYEETGNYLTKYSKKDNGPVIKKIKY | |
| | AAGCCCACCCTGAAGCAGATCGCCAAAGAAA | | YGNKLNAHLDITDDYPNSRNKVVKLSLKPY | |
| | TCCTCGTGAACGAAGAGGATATTAAGGGCTA | | RFDVYLDNGVYKFVTVKNLDVIKKENYYEV | |
| | CAGAGTGACCAGCACCGGCAAGCCCGAGTTC | | NSKCYEEAKKLKKISNQAEFIASFYNNDLI | |
| | ACCAACCTGAAGGTGTACCACGACATCAAGG | | KINGELYRVIGVNNDLLNRIEVNMIDITYR | |
| | ACATTACCGCCCGGAAAGAGATTATTGAGAA | | EYLENMNDKRPPRIIKTIASKTQSIKKYST | |
| | CGCCGAGCTGCTGGATCAGATTGCCAAGATC | | DILGNLYEVKSKKHPQIIKKGSGGSSGGSS | |
| | CTGACCATCTACCAGAGCAGCGAGGACATCC | | GSETPGTSESATPESSGGSSGGSSSGGSKR | |
| | AGGAAGAACTGACCAATCTGAACTCCGAGCT | | TADGSEFEPKKKRKVGSGATNFSLLKQAGD | |
| | GACCCAGGAAGAGATCGAGCAGATCTCTAAT | | VEENPGPMVSKGEELFTGVVPILVELDGDV | |
| | CTGAAGGGCTATACCGGCACCCACAACCTGA | | NGHKFSVSGEGEGDATYGKLILKFICTTGK | |
| | GCCTGAAGGCCATCAACCTGATCCTGGACGA | | LPVPWPTLVTTLTYGVQCFSRYPDHMKQHD | |
| | GCTGTGGCACACCAACGACAACCAGATCGCT | | FFKSAMPEGYVQERTIFFKDDGNYKTRAEV | |
| | ATCTTCAACCGGCTGAAGCTGGTGCCCAAGA | | KFEGDTLVNRIELKGIDFKEDGNILGHKLE | |
| | AGGTGGACCTGTCCCAGCAGAAAGAGATCCC | | YNYNSHNVYIMADKQKNGIKVNFKIRHNIE | |
| | CACCACCCTGGTGGACGACTTCATCCTGAGC | | DGSVQLADHYQQNTPIGDGPVLLPDNHYLS | |
| | CCCGTCGTGAAGAGAAGCTTCATCCAGAGCA | | TQSALSKDPNEKRDHMVLLEFVTAAGITLG | |
| | TCAAAGTGATCAACGCCATCATCAAGAAGTA | | MDELYK* | |
| | CGGCCTGCCCAACGACATCATTATCGAGCTG | | | |
| | GCCCGCGAGAAGAACTCCAAGGACGCCCAGA | | | |
| | AAATGATCAACGAGATGCAGAAGCGGAACCG | | | |
| | GCAGACCAACGAGCGGATCGAGGAAATCATC | | | |
| | CGGACCACCGGCAAAGAGAACGCCAAGTACC | | | |
| | TGATCGAGAAGATCAAGCTGCACGACATGCA | | | |
| | GGAAGGCAAGTGCCTGTACAGCCTGGAAGCC | | | |
| | ATCCCTCTGGAAGATCTGCTGAACAACCCCT | | | |
| | TCAACTATGAGGTGGACCACATCATCCCCAG | | | |
| | AAGCGTGTCCTTCGACAACAGCTTCAACAAC | | | |
| | AAGGTGCTCGTGAAGCAGGAAGAAGCCAGCA | | | |
| | AGAAGGGCAACCGGACCCCATTCCAGTACCT | | | |
| | GAGCAGCAGCGACAGCAAGATCAGCTACGAA | | | |
| | ACCTTCAAGAAGCACATCCTGAATCTGGCCA | | | |
| | AGGGCAAGGGCAGAATCAGCAAGACCAAGAA | | | |
| | AGAGTATCTGCTGGAAGAACGGGACATCAAC | | | |
| | AGGTTCTCCGTGCAGAAAGACTTCATCAACC | | | |
| | GGAACCTGGTGGATACCAGATACGCCACCAG | | | |
| | AGGCCTGATGAACCTGCTGCGGAGCTACTTC | | | |
| | AGAGTGAACAACCTGGACGTGAAAGTGAAGT | | | |
| | CCATCAATGGCGGCTTCACCAGCTTTCTGCG | | | |
| | GCGGAAGTGGAAGTTTAAGAAAGAGCGGAAC | | | |
| | AAGGGGTACAAGCACCACGCCGAGGACGCCC | | | |
| | TGATCATTGCCAACGCCGATTTCATCTTCAA | | | |
| | AGAGTGGAAGAAACTGGACAAGGCCAAAAAA | | | |
| | GTGATGGAAAACCAGATGTTCGAGGAAAAGC | | | |
| | AGGCCGAGAGCATGCCCGAGATCGAAACCGA | | | |
| | GCAGGAGTACAAAGAGATCTTCATCACCCCC | | | |
| | CACCAGATCAAGCACATTAAGGACTTCAAGG | | | |
| | ACTACAAGTACAGCCACCGGGTGGACAAGAA | | | |
| | GCCTAATAGAGAGCTGATTAACGACACCCTG | | | |
| | TACTCCACCCGGAAGGACGACAAGGGCAACA | | | |
| | CCCTGATCGTGAACAATCTGAACGGCCTGTA | | | |
| | CGACAAGGACAATGACAAGCTGAAAAAGCTG | | | |
| | ATCAACAAGAGCCCCGAAAAGCTGCTGATGT | | | |
| | ACCACCACGACCCCCAGACCTACCAGAAACT | | | |
| | GAAGCTGATTATGGAACAGTACGGCGACGAG | | | |
| | AAGAATCCCCTGTACAAGTACTACGAGGAAA | | | |
| | CCGGGAACTACCTGACCAAGTACTCCAAAAA | | | |
| | GGACAACGGCCCCGTGATCAAGAAGATTAAG | | | |
| | TATTACGGCAACAAACTGAACGCCCATCTGG | | | |
| | ACATCACCGACGACTACCCCAACAGCAGAAA | | | |
| | CAAGGTCGTGAAGCTGTCCCTGAAGCCCTAC | | | |
| | AGATTCGACGTGTACCTGGACAATGGCGTGT | | | |
| | ACAAGTTCGTGACCGTGAAGAATCTGGATGT | | | |
| | GATCAAAAAAGAAAACTACTACGAAGTGAAT | | | |
| | AGCAAGTGCTATGAGGAAGCTAAGAAGCTGA | | | |
| | AGAAGATCAGCAACCAGGCCGAGTTTATCGC | | | |
| | CTCCTTCTACAACAACGATCTGATCAAGATC | | | |
| | AACGGCGAGCTGTATAGAGTGATCGGCGTGA | | | |
| | ACAACGACCTGCTGAACCGGATCGAAGTGAA | | | |
| | CATGATCGACATCACCTACCGCGAGTACCTG | | | |
| | GAAAACATGAACGACAAGAGGCCCCCCAGGA | | | |
| | TCATTAAGACAATCGCCTCCAAGACCCAGAG | | | |
| | CATTAAGAAGTACAGCACAGACATTCTGGGC | | | |
| | AACCTGTATGAAGTGAAATCTAAGAAGCACC | | | |
| | CTCAGATCATCAAAAAGGGCTCTGGAGGATC | | | |
| | TAGCGGAGGATCCTCTGGCAGCGAGACACCA | | | |
| | GGAACAAGCGAGTCAGCAACACCAGAGAGCA | | | |
| | GTGGCGGCAGCAGCGGCGGCAGCAGCTCTGG | | | |
| | CGGCTCAAAAAGAACCGCCGACGGCAGCGAA | | | |
| | TTCGAGCCCAAGAAGAAGAGGAAAGTCGGAA | | | |
| | GCGGAGCTACTAACTTCAGCCTGCTGAAGCA | | | |
| | GGCTGGAGACGTGGAGGAGAACCCTGGACCT | | | |
| | ATGGTGAGCAAGGGCGAGGAGCTGTTCACCG | | | |
| | GGGTGGTGCCCATCCTGGTCGAGCTGGACGG | | | |
| | CGACGTAAACGGCCACAAGTTCAGCGTGTCC | | | |
| | GGCGAGGGCGAGGGCGATGCCACCTACGGCA | | | |
| | AGCTGACCCTGAAGTTCATCTGCACCACCGG | | | |
| | CAAGCTGCCCGTGCCCTGGCCCACCCTCGTG | | | |
| | ACCACCCTGACCTATGGAGTGCAGTGCTTCA | | | |
| | GCCGCTACCCCGACCACATGAAGCAGCACGA | | | |
| | CTTCTTCAAGTCCGCCATGCCCGAAGGCTAC | | | |
| | GTCCAGGAGCGCACCATCTTCTTCAAGGACG | | | |
| | ACGGCAACTACAAGACCCGCGCCGAGGTGAA | | | |
| | GTTCGAGGGCGACACCCTGGTGAACCGCATC | | | |
| | GAGCTGAAGGGCATCGACTTCAAGGAGGACG | | | |
| | GCAACATCCTGGGGCACAAGCTGGAGTACAA | | | |
| | CTACAACAGCCACAACGTCTATATCATGGCC | | | |
| | GACAAGCAGAAGAACGGCATCAAGGTGAACT | | | |
| | TCAAGATCCGCCACAACATCGAGGACGGCAG | | | |
| | CGTGCAGCTCGCCGACCACTACCAGCAGAAC | | | |
| | ACCCCCATCGGCGACGGCCCCGTGCTGCTGC | | | |
| | CCGACAACCACTACCTGAGCACCCAGTCCGC | | | |
| | CCTGAGCAAAGACCCCAACGAGAAGCGCGAT | | | |
| | CACATGGTCCTGCTGGAGTTCGTGACCGCCG | | | |
| | CCGGGATCACTCTCGGCATGGACGAGCTGTA | | | |
| | CAAGTAA | | | |
| bpNLS-nCas9 | ATGAAACGGACAGCCGACGGAAGCGAGTTCG | 102 | MKRTADGSEFESPKKKRKVDKKYSIGLDIG | 103 |
| (H840A)- | AGTCACCAAAGAAGAAGCGGAAAGTCGACAA | | TNSVGWAVITDEYKVPSKKFKVLGNTDRHS | |
| XTEN-MMLV | GAAGTACAGCATCGGCCTGGACATCGGCACC | | IKKNLIGALLFDSGETAEATRLKRTARRRY | |
| RT (dRH)- | AACTCTGTGGGCTGGGCCGTGATCACCGACG | | TRRKNRICYLQEIFSNEMAKVDDSFFHRLE | |
| 4 AA | AGTACAAGGTGCCCAGCAAGAAATTCAAGGT | | ESFLVEEDKKHERHPIFGNIVDEVAYHEKY | |
| linker- | GCTGGGCAACACCGACCGGCACAGCATCAAG | | PTIYHLRKKLVDSTDKADLRLIYLALAHMI | |
| bpNLS-P2A- | AAGAACCTGATCGGAGCCCTGCTGTTCGACA | | KFRGHFLIEGDLNPDNSDVDKLPIQLVQTY | |
| eGFP DELTA | GCGGCGAAACAGCCGAGGCCACCCGGCTGAA | | NQLFEENPINASGVDAKAILSARLSKSRRL | |
| RH FUSION | GAGAACCGCCAGAAGAAGATACACCAGACGG | | ENLIAQLPGEKKNGLFGNLIALSLGLTPNF | |
| | AAGAACCGGATCTGCTATCTGCAAGAGATCT | | KSNFDLAEDAKLQLSKDTYDDDLDNLLAQI | |
| | TCAGCAACGAGATGGCCAAGGTGGACGACAG | | GDQYADLFLAAKNLSDAILLSDILRVNTEI | |
| | CTTCTTCCACAGACTGGAAGAGTCCTTCCTG | | TKAPLSASMIKRYDEHHQDLTLLKALVRQQ | |
| | GTGGAAGAGGATAAGAAGCACGAGCGGCACC | | LPEKYKEIFFDQSKNGYAGYIDGGASQEEF | |
| | CCATCTTCGGCAACATCGTGGACGAGGTGGC | | YKFIKPILEKMDGTEELLVKLNREDLERKQ | |
| | CTACCACGAGAAGTACCCCACCATCTACCAC | | RTFDNGSIPHQIHLGELHAILRRQEDFYPF | |
| | CTGAGAAAGAAACTGGTGGACAGCACCGACA | | LKDNREKIEKILTFRIPYYVGPLARGNSRF | |
| | AGGCCGACCTGCGGCTGATCTATCTGGCCCT | | AWMTRKSEETITPWNFEEVVDKGASAQSPI | |
| | GGCCCACATGATCAAGTTCCGGGGCCACTTC | | ERMTNFDKNLPNEKVLPKHSLLYEYFTVYN | |
| | CTGATCGAGGGCGACCTGAACCCCGACAACA | | ELTKVKYVTEGMRKPAFLSGEQKKAIVDLL | |
| | GCGACGTGGACAAGCTGTTCATCCAGCTGGT | | FKTNRKVTVKQLKEDYFKKIECFDSVEISG | |
| | GCAGACCTACAACCAGCTGTTCGAGGAAAAC | | VEDRFNASLGTYHDLLKIIKDKDFLDNEEN | |
| | CCCATCAACGCCAGCGGCGTGGACGCCAAGG | | EDILEDIVLTLTLFEDREMIEERLKTYAHL | |
| | CCATCCTGTCTGCCAGACTGAGCAAGAGCAG | | FDDKVMKQLKRRRYTGWGRLSRKLINGIRD | |
| | ACGGCTGGAAAATCTGATCGCCCAGCTGCCC | | KQSGKTILDFLKSDGFANRNFMQLIHDDSL | |
| | GGCGAGAAGAAGAATGGCCTGTTCGGAAACC | | TFKEDIQKAQVSGQGDSLHEHIANLAGSPA | |
| | TGATTGCCCTGAGCCTGGGCCTGACCCCCAA | | IKKGILQTVKVVDELVKVMGRHKPENIVIE | |
| | CTTCAAGAGCAACTTCGACCTGGCCGAGGAT | | MARENQTTQKGQKNSRERMKRIEEGIKELG | |
| | GCCAAACTGCAGCTGAGCAAGGACACCTACG | | SQILKEHPVENTQLQNEKLYLYYLQNGRDM | |
| | ACGACGACCTGGACAACCTGCTGGCCCAGAT | | YVDQELDINRLSDYDVDAIVPQSFLKDDSI | |
| | CGGCGACCAGTACGCCGACCTGTTTCTGGCC | | DNKVLIRSDKNRGKSDNVPSEEVVKKMKNY | |
| | GCCAAGAACCTGTCCGACGCCATCCTGCTGA | | WRQLLNAKLITQRKEDNLTKAERGGLSELD | |
| | GCGACATCCTGAGAGTGAACACCGAGATCAC | | KAGFIKRQLVETRQITKHVAQILDSRMNTK | |
| | CAAGGCCCCCCTGAGCGCCTCTATGATCAAG | | YDENDKLIREVKVITLKSKLVSDERKDFQF | |
| | AGATACGACGAGCACCACCAGGACCTGACCC | | YKVREINNYHHAHDAYLNAVVGTALIKKYP | |
| | TGCTGAAAGCTCTCGTGCGGCAGCAGCTGCC | | KLESEFVYGDYKVYDVRKMIAKSEQEIGKA | |
| | TGAGAAGTACAAAGAGATTTTCTTCGACCAG | | TAKYFFYSNIMNFFKTEITLANGEIRKRPL | |
| | AGCAAGAACGGCTACGCCGGCTACATTGACG | | IETNGETGEIVWDKGRDFATVRKVLSMPQV | |
| | GCGGAGCCAGCCAGGAAGAGTTCTACAAGTT | | NIVKKTEVQTGGFSKESILPKRNSDKLIAR | |
| | CATCAAGCCCATCCTGGAAAAGATGGACGGC | | KKDWDPKKYGGFDSPTVAYSVLVVAKVEKG | |
| | ACCGAGGAACTGCTCGTGAAGCTGAACAGAG | | KSKKLKSVKELLGITIMERSSFEKNPIDEL | |
| | AGGACCTGCTGCGGAAGCAGCGGACCTTCGA | | EAKGYKEVKKDLIIKLPKYSLFELENGRKR | |
| | CAACGGCAGCATCCCCCACCAGATCCACCTG | | MLASAGELQKGNELALPSKYVNFLYLASHY | |
| | GGAGAGCTGCACGCCATTCTGCGGCGGCAGG | | EKLKGSPEDNEQKQLFVEQHKHYLDEIIEQ | |
| | AAGATTTTTACCCATTCCTGAAGGACAACCG | | ISEFSKRVILADANLDKVLSAYNKHRDKPI | |
| | GGAAAAGATCGAGAAGATCCTGACCTTCCGC | | REQAENIIHLFTLINLGAPAAFKYFDTTID | |
| | ATCCCCTACTACGTGGGCCCTCTGGCCAGGG | | RKRYTSTKEVLDATLIHQSITGLYETRIDL | |
| | GAAACAGCAGATTCGCCTGGATGACCAGAAA | | SQLGGDSGGSSGGSSGSETPGTSESATPES | |
| | GAGCGAGGAAACCATCACCCCCTGGAACTTC | | SGGSSGGSSTLNIEDEYRLHETSKEPDVSL | |
| | GAGGAAGTGGTGGACAAGGGCGCTTCCGCCC | | GSTWLSDFPQAWAETGGMGLAVRQAPLIIP | |
| | AGAGCTTCATCGAGCGGATGACCAACTTCGA | | LKATSTPVSIKQYPMSQEARLGIKPHIQRL | |
| | TAAGAACCTGCCCAACGAGAAGGTGCTGCCC | | LDQGILVPCQSPWNTPLLPVKKPGINDYRP | |
| | AAGCACAGCCTGCTGTACGAGTACTTCACCG | | VQDLREVNKRVEDIHPTVPNPYNLLSGLPP | |
| | TGTATAACGAGCTGACCAAAGTGAAATACGT | | SHQWYTVLDLKDAFFCLRLHPTSQPLFAFE | |
| | GACCGAGGGAATGAGAAAGCCCGCCTTCCTG | | WRDPEMGISGQLTWTRLPQGFKNSPTLFNE | |
| | AGCGGCGAGCAGAAAAAGGCCATCGTGGACC | | ALHRDLADFRIQHPDLILLQYVDDLLLAAT | |
| | TGCTGTTCAAGACCAACCGGAAAGTGACCGT | | SELDCQQGTRALLQTLGNLGYRASAKKAQI | |
| | GAAGCAGCTGAAAGAGGACTACTTCAAGAZA | | CQKQVKYLGYLLKEGQRWLTEARKETVMGQ | |
| | ATCGAGTGCTTCGACTCCGTGGAAATCTCCG | | PTPKTPRQLREFLGKAGFCRLFIPGFAEMA | |
| | GCGTGGAAGATCGGTTCAACGCCTCCCTGGG | | APLYPLIKPGTLFNWGPDQQKAYQEIKQAL | |
| | CACATACCACGATCTGCTGAAAATTATCAAG | | LTAPALGLPDLTKPFELFVDEKQGYAKGVL | |
| | GACAAGGACTTCCTGGACAATGAGGAAAACG | | TQKLGPWRRPVAYLSKKLDPVAAGWPPCLR | |
| | AGGACATTCTGGAAGATATCGTGCTGACCCT | | MVAAIAVLTKDAGKLTMGQPLVILAPHAVE | |
| | GACACTGTTTGAGGACAGAGAGATGATCGAG | | ALVKQPPDRWLSNARMTHYQALLLDTDRVQ | |
| | GAACGGCTGAAAACCTATGCCCACCTGTTCG | | FGPVVALNPATLLPLPEEGLQHNCLSGGSK | |
| | ACGACAAAGTGATGAAGCAGCTGAAGCGGCG | | RTADGSEFEPKKKRKVGSGATNFSLLKQAG | |
| | GAGATACACCGGCTGGGGCAGGCTGAGCCGG | | DVEENPGPMVSKGEELFTGVVPILVELDGD | |
| | AAGCTGATCAACGGCATCCGGGACAAGCAGT | | VNGHKFSVSGEGEGDATYGKLTLKFICTTG | |
| | CCGGCAAGACAATCCTGGATTTCCTGAAGTC | | KLPVPWPTLVTTLTYGVQCFSRYPDHMKQH | |
| | CGACGGCTTCGCCAACAGAAACTTCATGCAG | | DFFKSAMPEGYVQERTIFFKDDGNYKTRAE | |
| | CTGATCCACGACGACAGCCTGACCTTTAAAG | | VKFEGDTLVNRIELKGIDFKEDGNILGHKL | |
| | AGGACATCCAGAAAGCCCAGGTGTCCGGCCA | | EYNYNSHNVYIMADKQKNGIKVNFKIRHNI | |
| | GGGCGATAGCCTGCACGAGCACATTGCCAAT | | EDGSVQLADHYQQNTPIGDGPVLLPDNHYL | |
| | CTGGCCGGCAGCCCCGCCATTAAGAAGGGCA | | STQSALSKDPNEKRDHMVLLEFVTAAGITL | |
| | TCCTGCAGACAGTGAAGGTGGTGGACGAGCT | | GMDELYK* | |
| | CGTGAAAGTGATGGGCCGGCACAAGCCCGAG | | | |
| | AACATCGTGATCGAAATGGCCAGAGAGAACC | | | |
| | AGACCACCCAGAAGGGACAGAAGAACAGCCG | | | |
| | CGAGAGAATGAAGCGGATCGAAGAGGGCATC | | | |
| | AAAGAGCTGGGCAGCCAGATCCTGAAAGAAC | | | |
| | ACCCCGTGGAAAACACCCAGCTGCAGAACGA | | | |
| | GAAGCTGTACCTGTACTACCTGCAGAATGGG | | | |
| | CGGGATATGTACGTGGACCAGGAACTGGACA | | | |
| | TCAACCGGCTGTCCGACTACGATGTGGACGC | | | |
| | TATCGTGCCTCAGAGCTTTCTGAAGGACGAC | | | |
| | TCCATCGACAACAAGGTGCTGACCAGAAGCG | | | |
| | ACAAGAACCGGGGCAAGAGCGACAACGTGCC | | | |
| | CTCCGAAGAGGTCGTGAAGAAGATGAAGAAC | | | |
| | TACTGGCGGCAGCTGCTGAACGCCAAGCTGA | | | |
| | TTACCCAGAGAAAGTTCGACAATCTGACCAA | | | |
| | GGCCGAGAGAGGCGGCCTGAGCGAACTGGAT | | | |
| | AAGGCCGGCTTCATCAAGAGACAGCTGGTGG | | | |
| | GACTTACTGCTGGCCGCCACTTCTGAGCTAG | | | |
| | ACTGCCAACAAGGTACTCGGGCCCTGTTACA | | | |
| | AACCCTAGGGAACCTCGGGTATCGGGCCTCG | | | |
| | GCCAAGAAAGCCCAAATTTGCCAGAAACAGG | | | |
| | TCAAGTATCTGGGGTATCTTCTAAAAGAGGG | | | |
| | TCAGAGATGGCTGACTGAGGCCAGAAAAGAG | | | |
| | ACTGTGATGGGGCAGCCTACTCCGAAGACCC | | | |
| | CTCGACAACTAAGGGAGTTCCTAGGGAAGGC | | | |
| | AGGCTTCTGTCGCCTCTTCATCCCTGGGTTT | | | |
| | GCAGAAATGGCAGCCCCCCTGTACCCTCTCA | | | |
| | CCAAACCGGGGACTCTGTTTAATTGGGGCCC | | | |
| | AGACCAACAAAAGGCCTATCAAGAAATCAAG | | | |
| | CAAGCTCTTCTAACTGCCCCAGCCCTGGGGT | | | |
| | TGCCAGATTTGACTAAGCCCTTTGAACTCTT | | | |
| | TGTCGACGAGAAGCAGGGCTACGCCAAAGGT | | | |
| | GTCCTAACGCAAAAACTGGGACCTTGGCGTC | | | |
| | GGCCGGTGGCCTACCTGTCCAAAAAGCTAGA | | | |
| | CCCAGTAGCAGCTGGGTGGCCCCCTTGCCTA | | | |
| | CGGATGGTAGCAGCCATTGCCGTACTGACAA | | | |
| | AGGATGCAGGCAAGCTAACCATGGGACAGCC | | | |
| | ACTAGTCATTCTGGCCCCCCATGCAGTAGAG | | | |
| | GCACTAGTCAAACAACCCCCCGACCGCTGGC | | | |
| | TTTCCAACGCCCGGATGACTCACTATCAGGC | | | |
| | CTTGCTTTTGGACACGGACCGGGTCCAGTTC | | | |
| | GGACCGGTGGTAGCCCTGAACCCGGCTACGC | | | |
| | TGCTCCCACTGCCTGAGGAAGGGCTGCAACA | | | |
| | CAACTGCCTTTCTGGCGGCTCAAAAAGAACC | | | |
| | GCCGACGGCAGCGAATTCGAGCCCAAGAAGA | | | |
| | AGAGGAAAGTCGGAAGCGGAGCTACTAACTT | | | |
| | CAGCCTGCTGAAGCAGGCTGGAGACGTGGAG | | | |
| | GAGAACCCTGGACCTATGGTGAGCAAGGGCG | | | |
| | AGGAGCTGTTCACCGGGGTGGTGCCCATCCT | | | |
| | GGTCGAGCTGGACGGCGACGTAAACGGCCAC | | | |
| | AAGTTCAGCGTGTCCGGCGAGGGCGAGGGCG | | | |
| | ATGCCACCTACGGCAAGCTGACCCTGAAGTT | | | |
| | CATCTGCACCACCGGCAAGCTGCCCGTGCCC | | | |
| | TGGCCCACCCTCGTGACCACCCTGACCTATG | | | |
| | GAGTGCAGTGCTTCAGCCGCTACCCCGACCA | | | |
| | CATGAAGCAGCACGACTTCTTCAAGTCCGCC | | | |
| | ATGCCCGAAGGCTACGTCCAGGAGCGCACCA | | | |
| | TCTTCTTCAAGGACGACGGCAACTACAAGAC | | | |
| | CCGCGCCGAGGTGAAGTTCGAGGGCGACACC | | | |
| | CTGGTGAACCGCATCGAGCTGAAGGGCATCG | | | |
| | ACTTCAAGGAGGACGGCAACATCCTGGGGCA | | | |
| | CAAGCTGGAGTACAACTACAACAGCCACAAC | | | |
| | GTCTATATCATGGCCGACAAGCAGAAGAACG | | | |
| | GCATCAAGGTGAACTTCAAGATCCGCCACAA | | | |
| | CATCGAGGACGGCAGCGTGCAGCTCGCCGAC | | | |
| | CACTACCAGCAGAACACCCCCATCGGCGACG | | | |
| | GCCCCGTGCTGCTGCCCGACAACCACTACCT | | | |
| | GAGCACCCAGTCCGCCCTGAGCAAAGACCCC | | | |
| | AACGAGAAGCGCGATCACATGGTCCTGCTGG | | | |
| | AGTTCGTGACCGCCGCCGGGATCACTCTCGG | | | |
| | CATGGACGAGCTGTACAAGTAA | | | |
| nSpCas9 | ATGAAACGGACAGCCGACGGAAGCGAGTTCG | 104 | MKRTADGSEFESPKKKRKVDKKYSIGLDIG | 105 |
| (H840A) | AGTCACCAAAGAAGAAGCGGAAAGTCGACAA | | TNSVGWAVITDEYKVPSKKFKVLGNTDRHS | |
| | GAAGTACAGCATCGGCCTGGACATCGGCACC | | IKKNLIGALLFDSGETAEATRLKRTARRRY | |
| | AACTCTGTGGGCTGGGCCGTGATCACCGACG | | TRRKNRICYLQEIFSNEMAKVDDSFFHRLE | |
| | AGTACAAGGTGCCCAGCAAGAAATTCAAGGT | | ESFLVEEDKKHERHPIFGNIVDEVAYHEKY | |
| | GCTGGGCAACACCGACCGGCACAGCATCAAG | | PTIYHERKKLVDSTDKADLRLIYLALAHMI | |
| | AAGAACCTGATCGGAGCCCTGCTGTTCGACA | | KFRGHFLIEGDLNPDNSDVDKLFIQLVQTY | |
| | GCGGCGAAACAGCCGAGGCCACCCGGCTGAA | | NQLFEENPINASGVDAKAILSARLSKSRRL | |
| | GAGAACCGCCAGAAGAAGATACACCAGACGG | | ENLIAQLPGEKKNGLFGNLIALSLGLTPNF | |
| | AAGAACCGGATCTGCTATCTGCAAGAGATCT | | KSNFDLAEDAKLQLSKDTYDDDLDNLLAQI | |
| | TCAGCAACGAGATGGCCAAGGTGGACGACAG | | GDQYADLFLAAKNLSDAILLSDILRVNTEI | |
| | CTTCTTCCACAGACTGGAAGAGTCCTTCCTG | | TKAPLSASMIKRYDEHHQDLTLLKALVRQQ | |
| | GTGGAAGAGGATAAGAAGCACGAGCGGCACC | | LPEKYKEIFFDQSKNGYAGYIDGGASQEEF | |
| | CCATCTTTGGCAATATCGTGGACGAGGTGGC | | YKFIKPILEKMDGTEELLVKLNREDLLRKQ | |
| | AAACCCGGCAGATCACAAAGCACGTGGCACA | | RTFDNGSIPHQIHLGELHAILRRQEDFYPF | |
| | GATCCTGGACTCCCGGATGAACACTAAGTAC | | LKDNREKIEKILTFRIPYYVGPLARGNSRF | |
| | GACGAGAATGACAAGCTGATCCGGGAAGTGA | | AWMTRKSEETITPWNFEEVVDKGASAQSFI | |
| | AAGTGATCACCCTGAAGTCCAAGCTGGTGTC | | ERMTNFDKNLPNEKVLPKHSLLYEYFTVYN | |
| | CGATTTCCGGAAGGATTTCCAGTTTTACAAA | | ELTKVKYVTEGMRKPAFLSGEQKKAIVDLL | |
| | GTGCGCGAGATCAACAACTACCACCACGCCC | | FKTNRKVTVKQLKEDYFKKIECFDSVEISG | |
| | ACGACGCCTACCTGAACGCCGTCGTGGGAAC | | VEDRFNASLGTYHDLLKIIKDKDFLDNEEN | |
| | CGCCCTGATCAAAAAGTACCCTAAGCTGGAA | | EDILEDIVLTLTLFEDREMIEERLKTYAHL | |
| | AGCGAGTTCGTGTACGGCGACTACAAGGTGT | | FDDKVMKQLKRRRYTGWGRLSRKLINGIRD | |
| | ACGACGTGCGGAAGATGATCGCCAAGAGCGA | | KQSGKTILDFLKSDGFANRNFMQLIHDDSL | |
| | GCAGGAAATCGGCAAGGCTACCGCCAAGTAC | | TFKEDIQKAQVSGQGDSLHEHIANLAGSPA | |
| | TTCTTCTACAGCAACATCATGAACTTTTTCA | | IKKGILQTVKVVDELVKVMGRHKPENIVIE | |
| | AGACCGAGATTACCCTGGCCAACGGCGAGAT | | MARENQTTQKGQKNSRERMKRIEEGIKELG | |
| | CCGGAAGCGGCCTCTGATCGAGACAAACGGC | | SQILKEHPVENTQLQNEKLYLYYLQNGRDM | |
| | GAAACCGGGGAGATCGTGTGGGATAAGGGCC | | YVDQELDINRLSDYDVAAIVPQSFLKDDSI | |
| | GGGATTTTGCCACCGTGCGGAAAGTGCTGAG | | DNKVLTRSDKARGKSDNVPSEEVVKKMKNY | |
| | CATGCCCCAAGTGAATATCGTGAAAAAGACC | | WRQLLNAKLITQRKFDNLTKAERGGLSELD | |
| | GAGGTGCAGACAGGCGGCTTCAGCAAAGAGT | | KAGFIKRQLVETRQITKHVAQILDSRMNTK | |
| | CTATCCTGCCCAAGAGGAACAGCGATAAGCT | | YDENDKLIREVKVITLKSKLVSDFRKDFQF | |
| | GATCGCCAGAAAGAAGGACTGGGACCCTAAG | | YKVREINNYHHAHDAYLNAVVGTALIKKYP | |
| | AAGTACGGCGGCTTCGACAGCCCCACCGTGG | | KLESEFVYGDYKVYDVRKMIAKSEQEIGKA | |
| | CCTATTCTGTGCTGGTGGTGGCCAAAGTGGA | | TAKYFFYSNIMNFFKTEITLANGEIRKRPL | |
| | AAAGGGCAAGTCCAAGAAACTGAAGAGTGTG | | IETNGETGEIVWDKGRDFATVRKVLSMPQV | |
| | AAAGAGCTGCTGGGGATCACCATCATGGAAA | | NIVKKTEVQTGGFSKESILPKRNSDKLIAR | |
| | GAAGCAGCTTCGAGAAGAATCCCATCGACTT | | KKDWDPKKYGGFDSPTVAYSVLVVAKVEKG | |
| | TCTGGAAGCCAAGGGCTACAAAGAAGTGAAA | | KSKKLKSVKELLGITIMERSSPEKNPIDEL | |
| | AAGGACCTGATCATCAAGCTGCCTAAGTACT | | EAKGYKEVKKDLIIKLPKYSLFELENGRKR | |
| | CCCTGTTCGAGCTGGAAAACGGCCGGAAGAG | | MLASAGELQKGNELALPSKYVNFLYLASHY | |
| | AATGCTGGCCTCTGCCGGCGAACTGCAGAAG | | EKLKGSPEDNEQKQLFVEQHKHYLDEIIEQ | |
| | GGAAACGAACTGGCCCTGCCCTCCAAATATG | | ISEFSKRVILADANLDKVLSAYNKHRDKPI | |
| | TGAACTTCCTGTACCTGGCCAGCCACTATGA | | REQAENIIHLFTLINLGAPAAFKYFDTTID | |
| | GAAGCTGAAGGGCTCCCCCGAGGATAATGAG | | RKRYTSTKEVLDATLIHQSITGLYETRIDL | |
| | CAGAAACAGCTGTTTGTGGAACAGCACAAGC | | SQLGGDSPKKKRKVEAS* | |
| | ACTACCTGGACGAGATCATCGAGCAGATCAG | | | |
| | CGAGTTCTCCAAGAGAGTGATCCTGGCCGAC | | | |
| | GCTAATCTGGACAAAGTGCTGTCCGCCTACA | | | |
| | ACAAGCACCGGGATAAGCCCATCAGAGAGCA | | | |
| | GGCCGAGAATATCATCCACCTGTTTACCCTG | | | |
| | ACCAATCTGGGAGCCCCTGCCGCCTTCAAGT | | | |
| | ACTTTGACACCACCATCGACCGGAAGAGGTA | | | |
| | CACCAGCACCAAAGAGGTGCTGGACGCCACC | | | |
| | CTGATCCACCAGAGCATCACCGGCCTGTACG | | | |
| | AGACACGGATCGACCTGTCTCAGCTGGGAGG | | | |
| | TGACTCTGGAGGATCTAGCGGAGGATCCTCT | | | |
| | GGCAGCGAGACACCAGGAACAAGCGAGTCAG | | | |
| | CAACACCAGAGAGCAGTGGCGGCAGCAGCGG | | | |
| | CGGCAGCAGCACCCTAAATATAGAAGATGAG | | | |
| | TATCGGCTACATGAGACCTCAAAAGAGCCAG | | | |
| | ATGTTTCTCTAGGGTCCACATGGCTGTCTGA | | | |
| | TTTTCCTCAGGCCTGGGCGGAAACCGGGGGC | | | |
| | ATGGGACTGGCAGTTCGCCAAGCTCCTCTGA | | | |
| | TCATACCTCTGAAAGCAACCTCTACCCCCGT | | | |
| | GTCCATAAAACAATACCCCATGTCACAAGAA | | | |
| | GCCAGACTGGGGATCAAGCCCCACATACAGA | | | |
| | GACTGTTGGACCAGGGAATACTGGTACCCTG | | | |
| | CCAGTCCCCCTGGAACACGCCCCTGCTACCC | | | |
| | GTTAAGAAACCAGGGACTAATGATTATAGGC | | | |
| | CTGTCCAGGATCTGAGAGAAGTCAACAAGCG | | | |
| | GGTGGAAGACATCCACCCCACCGTGCCCAAC | | | |
| | CCTTACAACCTCTTGAGCGGGCTCCCACCGT | | | |
| | CCCACCAGTGGTACACTGTGCTTGATTTAAA | | | |
| | GGATGCCTTTTTCTGCCTGAGACTCCACCCC | | | |
| | ACCAGTCAGCCTCTCTTCGCCTTTGAGTGGA | | | |
| | GAGATCCAGAGATGGGAATCTCAGGACAATT | | | |
| | GACCTGGACCAGACTCCCACAGGGTTTCAAA | | | |
| | AACAGTCCCACCCTGTTTAATGAGGCACTGC | | | |
| | ACAGAGACCTAGCAGACTTCCGGATCCAGCA | | | |
| | CCCAGACTTGATCCTGCTACAGTACGTGGAT | | | |
| | GTACCATGAAAAGTACCCAACCATATATCAT | | | |
| | CTGAGGAAGAAGCTTGTAGACAGTACTGATA | | | |
| | AGGCTGACTTGCGGTTGATCTATCTCGCGCT | | | |
| | GGCGCATATGATCAAATTTCGGGGACACTTC | | | |
| | CTCATCGAGGGGGACCTGAACCCAGACAACA | | | |
| | GCGATGTCGACAAACTCTTTATCCAACTGGT | | | |
| | TCAGACTTACAATCAGCTTTTCGAAGAGAAC | | | |
| | CCGATCAACGCATCCGGAGTTGACGCCAAAG | | | |
| | CAATCCTGAGCGCTAGGCTGTCCAAATCCCG | | | |
| | GCGGCTCGAAAACCTCATCGCACAGCTCCCT | | | |
| | GGGGAGAAGAAGAACGGCCTGTTTGGTAATC | | | |
| | TTATCGCCCTGTCACTCGGGCTGACCCCCAA | | | |
| | CTTTAAATCTAACTTCGACCTGGCCGAAGAT | | | |
| | GCCAAGCTTCAACTGAGCAAAGACACCTACG | | | |
| | ATGATGATCTCGACAATCTGCTGGCCCAGAT | | | |
| | CGGCGACCAGTACGCAGACCTTTTTTTGGCG | | | |
| | GCAAAGAACCTGTCAGACGCCATTCTGCTGA | | | |
| | GTGATATTCTGCGAGTGAACACGGAGATCAC | | | |
| | CAAAGCTCCGCTGAGCGCTAGTATGATCAAG | | | |
| | CGCTATGATGAGCACCACCAAGACTTGACTT | | | |
| | TGCTGAAGGCCCTTGTCAGACAGCAACTGCC | | | |
| | TGAGAAGTACAAGGAAATTTTCTTCGATCAG | | | |
| | TCTAAAAATGGCTACGCCGGATACATTGACG | | | |
| | GCGGAGCAAGCCAGGAGGAATTTTACAAATT | | | |
| | TATTAAGCCCATCTTGGAAAAAATGGACGGC | | | |
| | ACCGAGGAGCTGCTGGTAAAGCTTAACAGAG | | | |
| | AAGATCTGTTGCGCAAACAGCGCACTTTCGA | | | |
| | CAATGGAAGCATCCCCCACCAGATTCACCTG | | | |
| | GGCGAACTGCACGCTATCCTCAGGCGGCAAG | | | |
| | AGGATTTCTACCCCTTTTTGAAAGATAACAG | | | |
| | GGAAAAGATTGAGAAAATCCTCACATTTCGG | | | |
| | ATACCCTACTATGTAGGCCCCCTCGCCCGGG | | | |
| | GAAATTCCAGATTCGCGTGGATGACTCGCAA | | | |
| | ATCAGAAGAGACCATCACTCCCTGGAACTTC | | | |
| | GAGGAAGTCGTGGATAAGGGGGCCTCTGCCC | | | |
| | AGTCCTTCATCGAAAGGATGACTAACTTTGA | | | |
| | TAAAAATCTGCCTAACGAAAAGGTGCTTCCT | | | |
| | AAACACTCTCTGCTGTACGAGTACTTCACAG | | | |
| | TTTATAACGAGCTCACCAAGGTCAAATACGT | | | |
| | CACAGAAGGGATGAGAAAGCCAGCATTCCTG | | | |
| | TCTGGAGAGCAGAAGAAAGCTATCGTGGACC | | | |
| | TCCTCTTCAAGACGAACCGGAAAGTTACCGT | | | |
| | GAAACAGCTCAAAGAAGACTATTTCAAAAAG | | | |
| | ATTGAATGTTTCGACTCTGTTGAAATCAGCG | | | |
| | GAGTGGAGGATCGCTTCAACGCATCCCTGGG | | | |
| | AACGTATCACGATCTCCTGAAAATCATTAAA | | | |
| | GACAAGGACTTCCTGGACAATGAGGAGAACG | | | |
| | AGGACATTCTTGAGGACATTGTCCTCACCCT | | | |
| | TACGTTGTTTGAAGATAGGGAGATGATTGAA | | | |
| | GAACGCTTGAAAACTTACGCTCATCTCTTCG | | | |
| | ACGACAAAGTCATGAAACAGCTCAAGAGGCG | | | |
| | CCGATATACAGGATGGGGGCGGCTGTCAAGA | | | |
| | AAACTGATCAATGGGATCCGAGACAAGCAGA | | | |
| | GTGGAAAGACAATCCTGGATTTTCTTAAGTC | | | |
| | CGATGGATTTGCCAACCGGAACTTCATGCAG | | | |
| | TTGATCCATGATGACTCTCTCACCTTTAAGG | | | |
| | AGGACATCCAGAAAGCACAAGTTTCTGGCCA | | | |
| | GGGGGACAGTCTTCACGAGCACATCGCTAAT | | | |
| | CTTGCAGGTAGCCCAGCTATCAAAAAGGGAA | | | |
| | TACTGCAGACCGTTAAGGTCGTGGATGAACT | | | |
| | CGTCAAAGTAATGGGAAGGCATAAGCCCGAG | | | |
| | AATATCGTTATCGAGATGGCCCGAGAGAACC | | | |
| | AAACTACCCAGAAGGGACAGAAGAACAGTAG | | | |
| | GGAAAGGATGAAGAGGATTGAAGAGGGTATA | | | |
| | AAAGAACTGGGGTCCCAAATCCTTAAGGAAC | | | |
| | ACCCAGTTGAAAACACCCAGCTTCAGAATGA | | | |
| | GAAGCTCTACCTGTACTACCTGCAGAACGGC | | | |
| | AGGGACATGTACGTGGATCAGGAACTGGACA | | | |
| | TCAATCGGCTCTCCGACTACGACGTGGCTGC | | | |
| | TATCGTGCCCCAGTCTTTTCTCAAAGATGAT | | | |
| | TCTATTGATAATAAAGTGTTGACAAGATCCG | | | |
| | ATAAAGCTAGAGGGAAGAGTGATAACGTCCC | | | |
| | CTCAGAAGAAGTTGTCAAGAAAATGAAAAAT | | | |
| | TATTGGCGGCAGCTGCTGAACGCCAAACTGA | | | |
| | TCACACAACGGAAGTTCGATAATCTGACTAA | | | |
| | GGCTGAACGAGGTGGCCTGTCTGAGTTGGAT | | | |
| | AAAGCCGGCTTCATCAAAAGGCAGCTTGTTG | | | |
| | AGACACGCCAGATCACCAAGCACGTGGCCCA | | | |
| | AATTCTCGATTCACGCATGAACACCAAGTAC | | | |
| | GATGAAAATGACAAACTGATTCGAGAGGTGA | | | |
| | AAGTTATTACTCTGAAGTCTAAGCTGGTCTC | | | |
| | AGATTTCAGAAAGGACTTTCAGTTTTATAAG | | | |
| | GTGAGAGAGATCAACAATTACCACCATGCGC | | | |
| | ATGATGCCTACCTGAATGCAGTGGTAGGCAC | | | |
| | TGCACTTATCAAAAAATATCCCAAGCTTGAA | | | |
| | TCTGAATTTGTTTACGGAGACTATAAAGTGT | | | |
| | ACGATGTTAGGAAAATGATCGCAAAGTCTGA | | | |
| | GCAGGAAATAGGCAAGGCCACCGCTAAGTAC | | | |
| | TTCTTTTACAGCAATATTATGAATTTTTTCA | | | |
| | AGACCGAGATTACACTGGCCAATGGAGAGAT | | | |
| | TCGGAAGCGACCACTTATCGAAACAAACGGA | | | |
| | GAAACAGGAGAAATCGTGTGGGACAAGGGTA | | | |
| | GGGATTTCGCGACAGTCCGGAAGGTCCTGTC | | | |
| | CATGCCGCAGGTGAACATCGTTAAAAAGACC | | | |
| | GAAGTACAGACCGGAGGCTTCTCCAAGGAAA | | | |
| | GTATCCTCCCGAAAAGGAACAGCGACAAGCT | | | |
| | GATCGCACGCAAAAAAGATTGGGACCCCAAG | | | |
| | AAATACGGCGGATTCGATTCTCCTACAGTCG | | | |
| | CTTACAGTGTACTGGTTGTGGCCAAAGTGGA | | | |
| | GAAAGGGAAGTCTAAAAAACTCAAAAGCGTC | | | |
| | AAGGAACTGCTGGGCATCACAATCATGGAGC | | | |
| | GATCAAGCTTCGAAAAAAACCCCATCGACTT | | | |
| | TCTCGAGGCGAAAGGATATAAAGAGGTCAAA | | | |
| | AAAGACCTCATCATTAAGCTTCCCAAGTACT | | | |
| | CTCTCTTTGAGCTTGAAAACGGCCGGAAACG | | | |
| | AATGCTCGCTAGTGCGGGCGAGCTGCAGAAA | | | |
| | GGTAACGAGCTGGCACTGCCCTCTAAATACG | | | |
| | TTAATTTCTTGTATCTGGCCAGCCACTATGA | | | |
| | AAAGCTCAAAGGGTCTCCCGAAGATAATGAG | | | |
| | CAGAAGCAGCTGTTCGTGGAACAACACAAAC | | | |
| | ACTACCTTGATGAGATCATCGAGCAAATAAG | | | |
| | CGAATTCTCCAAAAGAGTGATCCTCGCCGAC | | | |
| | GCTAACCTCGATAAGGTGCTTTCTGCTTACA | | | |
| | ATAAGCACAGGGATAAGCCCATCAGGGAGCA | | | |
| | GGCAGAAAACATTATCCACTTGTTTACTCTG | | | |
| | ACCAACTTGGGCGCGCCTGCAGCCTTCAAGT | | | |
| | ACTTCGACACCACCATAGACAGAAAGCGGTA | | | |
| | CACCTCTACAAAGGAGGTCCTGGACGCCACA | | | |
| | CTGATTCATCAGTCAATTACGGGGCTCTATG | | | |
| | AAACAAGAATCGACCTCTCTCAGCTCGGTGG | | | |
| | AGACAGCCCCAAGAAGAAGAGAAAGGTGGAG | | | |
| | GCCAGCTAA | | | |
| pegRNA- | TGTGGACTACTAGTAAGCTTGGATCTTGAAG | 106 | CGLLVSLDLEEAAGGAGGEVVRIRSV*GSV | 107 |
| pH1- | AAGCTGCAGGAGGTGCTGGAGGGGAAGTGGT | | KLV*GPISHDSFIFAYTIQGC*RDN*N*FD | |
| ngRNA- | CCGGATCCGATCAGTGTGAGGGAGTGTAAAG | | CKHKDISTKYVT*KVIISWVVCSFKIMF*N | |
| pEPS- | CTGGTTTGAGGGCCTATTTCCCATGATTCCT | | GLSYAYRNLKVFRFLGFIYLVERTKHRPRL | |
| bpNLS- | TCATATTTGCATATACGATACAAGGCTGTTA | | ST*VLELEIAS*NKASPLST*KSGTESVLC | |
| MMLVRT | GAGAGATAATTAGAATTAATTTGACTGTAAA | | HQSVLSLFFWNSNADVINPLQGIAGPVSLG | |
| (dRH)- | CACAAAGATATTAGTACAAAATACGTGACGT | | GNTQRACALAGRWL*GTGEWRPAIFACRYV | |
| bpNLS- | AGAAAGTAATAATTTCTTGGGTAGTTTGCAG | | FWEITINVKCLWIWESYKFCMRPLFPVNQY | |
| | TTTTAAAATTATGTTTTAAAATGGACTATCA | | PGAF*S*K*QVKIRLVRYQLEKVAPSRCFF | |
| | TATGCTTACCGTAACTTGAAAGTATTTCGAT | | SNSNASCAIVFEWLRCPSVGRAHIAHSPRE | |
| | TTCTTGGCTTTATATATCTTGTGGAAAGGAC | | VGGRGRQLNRCLEKVARGKLGK*CRVLAPP | |
| | GAAACACCGGCCCAGACTGAGCACGTGAGTT | | ESRGWGRTVYKCSSRRERSFSQRVCRQNTG | |
| | TTAGAGCTAGAAATAGCAAGTTAAAATAAGG | | VVTRDPTLALQLKRAATMKRTADGSEFESP | |
| | CTAGTCCGTTATCAACTTGAAAAAGTGGGAC | | KKKRKVILNIEDEYRLHETSKEPDVSIGST | |
| | CGAGTCGGTCCTCTGCCATCAAAGCGTGCTC | | WLSDFPQAWAETGGMGLAVRQAPLIIPLKA | |
| | AGTCTGTTTTTTTGGAATTCGAACGCTGACG | | TSTPVSIKQYPMSQEARLGIKPHIQRLLDQ | |
| | TCATCAACCCGCTCCAAGGAATCGCGGGCCC | | GILVPCQSPWNTPLLPVKKPGTNDYRPVQD | |
| | AGTGTCACTAGGCGGGAACACCCAGCGCGCG | | LREVNKRVEDIHPTVPNPYNLLSGLPPSHQ | |
| | TGCGCCCTGGCAGGAAGATGGCTGTGAGGGA | | WYTVLDLKDAFFCERLAPTSQPLFAFEWRD | |
| | CAGGGGAGTGGCGCCCTGCAATATTTGCATG | | PEMGISGQLTWTRLPQGFKNSPTLFNEALH | |
| | TCGCTATGTGTTCTGGGAAATCACCATAAAC | | RDLADFRIQHPDLILLQYVDDLLLAATSEL | |
| | GTGAAATGTCTTTGGATTTGGGAATCTTATA | | DCQQGTRALLQTEGNLGYRASAKKAQICQK | |
| | AGTTCTGTATGAGACCACTTTTTCCCGTCAA | | QVKYLGYLLKEGQRWLTEARKETVMGQPTP | |
| | CCAGTATCCCGGTGCGTTTTAGAGCTAGAAA | | KTPRQLREFLGKAGFCRLFIPGFAEMAAPL | |
| | TAGCAAGTTAAAATAAGGCTAGTCCGTTATC | | YPLTKPGTLFNWGPDQQKAYQEIKQALLTA | |
| | AACTTGAAAAAGTGGCACCGAGTCGGTGCTT | | PALGLPDLTKPFELFVDEKQGYAKGVLTQK | |
| | TTTTTCTAACTCGAACGCTAGCTGTGCGATC | | LGPWRRPVAYLSKKLDPVAAGWPPCLRMVA | |
| | GTTTTCGAGTGGCTCCGGTGCCCGTCAGTGG | | AIAVLIKDAGKLIMGQPLVILAPHAVEALV | |
| | GCAGAGCGCACATCGCCCACAGTCCCCGAGA | | KQPPDRWLSNARMTHYQALLLDTDRVQFGP | |
| | AGTTGGGGGGAGGGGTCGGCAATTGAACCGG | | VVALNPATLLPLPEEGLQHNCLSGGSKRTA | |
| | TGCCTAGAGAAGGTGGCGCGGGGTAAACTGG | | DGSEFEPKKKRKVGSGATNFSLLKQAGDVE | |
| | GAAAGTGATGTCGTGTACTGGCTCCGCCTTT | | ENPGPMVSKGEELFTGVVPILVELDGDVNG | |
| | TTCCCGAGGGTGGGGGAGAACCGTATATAAG | | HKFSVSGEGEGDATYGKLTLKPICTTGKLP | |
| | TGCAGTAGTCGCCGTGAACGTTCTTTTTCGC | | VPWPTLVTTLTYGVQCFSRYPDHMKQHDFF | |
| | AACGGGTTTGCCGCCAGAACACAGGTGTCGT | | KSAMPEGYVQERTIFFKDDGNYKTRAEVKF | |
| | GACGCGGGACCCGACATTAGCGCTACAGCTT | | EGDTLVNRIELKGIDFKEDGNILGHKLEYN | |
| | AAGCGGGCCGCCACCATGAAACGGACAGCCG | | YNSHNVYIMADKQKNGIKVNFKIRHNIEDG | |
| | ACGGAAGCGAGTTCGAGTCACCAAAGAAGAA | | SVQLADHYQQNTPIGDGPVLLPDNHYLSTQ | |
| | GCGGAAAGTCACCCTAAATATAGAAGATGAG | | SALSKDPNEKRDHMVLLEFVTAAGITLGMD | |
| | TATCGGCTACATGAGACCTCAAAAGAGCCAG | | ELYK* | |
| | ATGTTTCTCTAGGGTCCACATGGCTGTCTGA | | | |
| | TTTTCCTCAGGCCTGGGCGGAAACCGGGGGC | | | |
| | ATGGGACTGGCAGTTCGCCAAGCTCCTCTGA | | | |
| | TCATACCTCTGAAAGCAACCTCTACCCCCGT | | | |
| | GTCCATAAAACAATACCCCATGTCACAAGAA | | | |
| | GCCAGACTGGGGATCAAGCCCCACATACAGA | | | |
| | GACTGTTGGACCAGGGAATACTGGTACCCTG | | | |
| | CCAGTCCCCCTGGAACACGCCCCTGCTACCC | | | |
| | GTTAAGAAACCAGGGACTAATGATTATAGGC | | | |
| | CTGTCCAGGATCTGAGAGAAGTCAACAAGCG | | | |
| | GGTGGAAGACATCCACCCCACCGTGCCCAAC | | | |
| | CCTTACAACCTCTTGAGCGGGCTCCCACCGT | | | |
| | CCCACCAGTGGTACACTGTGCTTGATTTAAA | | | |
| | GGATGCCTTTTTCTGCCTGAGACTCCACCCC | | | |
| | ACCAGTCAGCCTCTCTTCGCCTTTGAGTGGA | | | |
| | GAGATCCAGAGATGGGAATCTCAGGACAATT | | | |
| | GACCTGGACCAGACTCCCACAGGGTTTCAAA | | | |
| | AACAGTCCCACCCTGTTTAATGAGGCACTGC | | | |
| | ACAGAGACCTAGCAGACTTCCGGATCCAGCA | | | |
| | CCCAGACTTGATCCTGCTACAGTACGTGGAT | | | |
| | GACTTACTGCTGGCCGCCACTTCTGAGCTAG | | | |
| | ACTGCCAACAAGGTACTCGGGCCCTGTTACA | | | |
| | AACCCTAGGGAACCTCGGGTATCGGGCCTCG | | | |
| | GCCAAGAAAGCCCAAATTTGCCAGAAACAGG | | | |
| | TCAAGTATCTGGGGTATCTTCTAAAAGAGGG | | | |
| | TCAGAGATGGCTGACTGAGGCCAGAAAAGAG | | | |
| | ACTGTGATGGGGCAGCCTACTCCGAAGACCC | | | |
| | CTCGACAACTAAGGGAGTTCCTAGGGAAGGC | | | |
| | AGGCTTCTGTCGCCTCTTCATCCCTGGGTTT | | | |
| | GCAGAAATGGCAGCCCCCCTGTACCCTCTCA | | | |
| | CCAAACCGGGGACTCTGTTTAATTGGGGCCC | | | |
| | AGACCAACAAAAGGCCTATCAAGAAATCAAG | | | |
| | CAAGCTCTTCTAACTGCCCCAGCCCTGGGGT | | | |
| | TGCCAGATTTGACTAAGCCCTTTGAACTCTT | | | |
| | TGTCGACGAGAAGCAGGGCTACGCCAAAGGT | | | |
| | GTCCTAACGCAAAAACTGGGACCTTGGCGTC | | | |
| | GGCCGGTGGCCTACCTGTCCAAAAAGCTAGA | | | |
| | CCCAGTAGCAGCTGGGTGGCCCCCTTGCCTA | | | |
| | CGGATGGTAGCAGCCATTGCCGTACTGACAA | | | |
| | AGGATGCAGGCAAGCTAACCATGGGACAGCC | | | |
| | ACTAGTCATTCTGGCCCCCCATGCAGTAGAG | | | |
| | GCACTAGTCAAACAACCCCCCGACCGCTGGC | | | |
| | TTTCCAACGCCCGGATGACTCACTATCAGGC | | | |
| | CTTGCTTTTGGACACGGACCGGGTCCAGTTC | | | |
| | GGACCGGTGGTAGCCCTGAACCCGGCTACGC | | | |
| | TGCTCCCACTGCCTGAGGAAGGGCTGCAACA | | | |
| | CAACTGCCTTTCTGGCGGCTCAAAAAGAACC | | | |
| | GCCGACGGCAGCGAATTCGAGCCCAAGAAGA | | | |
| | AGAGGAAAGTCGGAAGCGGAGCTACTAACTT | | | |
| | CAGCCTGCTGAAGCAGGCTGGAGACGTGGAG | | | |
| | GAGAACCCTGGACCTATGGTGAGCAAGGGCG | | | |
| | AGGAGCTGTTCACCGGGGTGGTGCCCATCCT | | | |
| | GGTCGAGCTGGACGGCGACGTAAACGGCCAC | | | |
| | AAGTTCAGCGTGTCCGGCGAGGGCGAGGGCG | | | |
| | ATGCCACCTACGGCAAGCTGACCCTGAAGTT | | | |
| | CATCTGCACCACCGGCAAGCTGCCCGTGCCC | | | |
| | TGGCCCACCCTCGTGACCACCCTGACCTATG | | | |
| | GAGTGCAGTGCTTCAGCCGCTACCCCGACCA | | | |
| | CATGAAGCAGCACGACTTCTTCAAGTCCGCC | | | |
| | ATGCCCGAAGGCTACGTCCAGGAGCGCACCA | | | |
| | TCTTCTTCAAGGACGACGGCAACTACAAGAC | | | |
| | CCGCGCCGAGGTGAAGTTCGAGGGCGACACC | | | |
| | CTGGTGAACCGCATCGAGCTGAAGGGCATCG | | | |
| | ACTTCAAGGAGGACGGCAACATCCTGGGGCA | | | |
| | CAAGCTGGAGTACAACTACAACAGCCACAAC | | | |
| | GTCTATATCATGGCCGACAAGCAGAAGAACG | | | |
| | GCATCAAGGTGAACTTCAAGATCCGCCACAA | | | |
| | CATCGAGGACGGCAGCGTGCAGCTCGCCGAC | | | |
| | CACTACCAGCAGAACACCCCCATCGGCGACG | | | |
| | GCCCCGTGCTGCTGCCCGACAACCACTACCT | | | |
| | GAGCACCCAGTCCGCCCTGAGCAAAGACCCC | | | |
| | AACGAGAAGCGCGATCACATGGTCCTGCTGG | | | |
| | AGTTCGTGACCGCCGCCGGGATCACTCTCGG | | | |
| | CATGGACGAGCTGTACAAGTAA | | | |
| bpNLS- | ATGAAACGGACAGCCGACGGAAGCGAGTTCG | 108 | MKRTADGSEFESPKKKRKVDKKYSIGLDIG | 109 |
| nCas9 | AGTCACCAAAGAAGAAGCGGAAAGTCGACAA | | TNSVGWAVITDEYKVPSKKFKVLGNTDRHS | |
| (H840A)- | GAAGTACAGCATCGGCCTGGACATCGGCACC | | IKKNLIGALLFDSGETAEATRLKRTARRRY | |
| XTEN- | AACTCTGTGGGCTGGGCCGTGATCACCGACG | | TRRKNRICYLQEIFSNEMAKVDDSFFHRLE | |
| Marathon | AGTACAAGGTGCCCAGCAAGAAATTCAAGGT | | ESFLVEEDKKHERHPIFGNIVDEVAYHEKY | |
| RT | GCTGGGCAACACCGACCGGCACAGCATCAAG | | PTIYHERKKLVDSTDKADLRLIYLALAHMI | |
| (D14R- | AAGAACCTGATCGGAGCCCTGCTGTTCGACA | | KFRGHFLIEGDLNPDNSDVDKLFIQLVQTY | |
| N26R- | GCGGCGAAACAGCCGAGGCCACCCGGCTGAA | | NQLFEENPINASGVDAKAILSARLSKSRRL | |
| D74R- | GAGAACCGCCAGAAGAAGATACACCAGACGG | | ENLIAQLPGEKKNGLFGNLIALSLGLQPNE | |
| N116K- | AAGAACCGGATCTGCTATCTGCAAGAGATCT | | KSNFDLAEDAKLQLSKDTYDDDLDNLLAQI | |
| N197R)- | TCAGCAACGAGATGGCCAAGGTGGACGACAG | | GDQYADLFLAAKNLSDAILLSDILRVNTEI | |
| 4 AA | CTTCTTCCACAGACTGGAAGAGTCCTTCCTG | | TKAPLSASMIKRYDEHHQDLTLLKALVRQQ | |
| linker- | GTGGAAGAGGATAAGAAGCACGAGCGGCACC | | LPEKYKEIFFDQSKNGYAGYIDGGASQEEF | |
| bpNLS- | CCATCTTCGGCAACATCGTGGACGAGGTGGC | | YKFIKPILEKMDGTEELLVKLNREDLLRKQ | |
| P2A-eGFP | CTACCACGAGAAGTACCCCACCATCTACCAC | | RTFDNGSIPHQIHLGELHAILRRQEDFYPF | |
| | CTGAGAAAGAAACTGGTGGACAGCACCGACA | | LKDNREKIEKILTFRIPYYVGPLARGNSRF | |
| | AGGCCGACCTGCGGCTGATCTATCTGGCCCT | | AWMTRKSEETITPWNFEEVVDKGASAQSFI | |
| | GGCCCACATGATCAAGTTCCGGGGCCACTTC | | ERMINFDKNLPNEKVLPKHSLLYEYFTVYN | |
| | CTGATCGAGGGCGACCTGAACCCCGACAACA | | ELTKVKYVTEGMRKPAFLSGEQKKAIVDLL | |
| | GCGACGTGGACAAGCTGTTCATCCAGCTGGT | | FKTNRKVTVKQLKEDYFKKIECFDSVEISG | |
| | GCAGACCTACAACCAGCTGTTCGAGGAAAAC | | VEDRFNASLGTYHDLLKIIKDKDELDNEEN | |
| | CCCATCAACGCCAGCGGCGTGGACGCCAAGG | | EDILEDIVLTLILFEDREMIEERLKTYAHL | |
| | CCATCCTGTCTGCCAGACTGAGCAAGAGCAG | | FDDKVMKQLKRRRYTGWGRLSRKLINGIRD | |
| | ACGGCTGGAAAATCTGATCGCCCAGCTGCCC | | KQSGKTILDFLKSDGFANRNEMQLIHDDSL | |
| | GGCGAGAAGAAGAATGGCCTGTTCGGAAACC | | TFKEDIQKAQVSGQGDSLHEHIANLAGSPA | |
| | TGATTGCCCTGAGCCTGGGCCTGACCCCCAA | | IKKGILQTVKVVDELVKVMGRHKPENIVIE | |
| | CTTCAAGAGCAACTTCGACCTGGCCGAGGAT | | MARENQTTQKGQKNSRERMKRIREGIKELG | |
| | GCCAAACTGCAGCTGAGCAAGGACACCTACG | | SQILKEHPVENTQLQNEKLYLYYLQNGRDM | |
| | ACGACGACCTGGACAACCTGCTGGCCCAGAT | | YVDQELDINRLSDYDVDAIVPQSFLKDDSI | |
| | CGGCGACCAGTACGCCGACCTGTTTCTGGCC | | DNKVLERSDKNRGKSDNVPSEEVVKKMKNY | |
| | GCCAAGAACCTGTCCGACGCCATCCTGCTGA | | WRQLLNAKLITQRKFDNLTKAERGGLSELD | |
| | GCGACATCCTGAGAGTGAACACCGAGATCAC | | KAGFIKRQLVETRQITKHVAQILDSRMNTK | |
| | CAAGGCCCCCCTGAGCGCCTCTATGATCAAG | | YDENDKLIREVKVITLKSKLVSDFRKDFQF | |
| | AGATACGACGAGCACCACCAGGACCTGACCC | | YKVREINNYHHAHDAYLNAVVGTALIKKYP | |
| | TGCTGAAAGCTCTCGTGCGGCAGCAGCTGCC | | KLESEFVYGDYKVYDVRKMIAKSEQEIGKA | |
| | TGAGAAGTACAAAGAGATTTTCTTCGACCAG | | TAKYFFYSNIMNFFKTEITLANGEIRKRPL | |
| | AGCAAGAACGGCTACGCCGGCTACATTGACG | | IETNGETGEIVWDKGRDFATVRKVLSMPQV | |
| | GCGGAGCCAGCCAGGAAGAGTTCTACAAGTT | | NIVKKTEVQTGGFSKESILPKRNSDKLIAR | |
| | CATCAAGCCCATCCTGGAAAAGATGGACGGC | | KKDWDPKKYGGFDSPTVAYSVLVVAKVEKG | |
| | ACCGAGGAACTGCTCGTGAAGCTGAACAGAG | | KSKKLKSVKELLGITIMERSSFEKNPIDEL | |
| | AGGACCTGCTGCGGAAGCAGCGGACCTTCGA | | EAKGYKEVKKDLIIKLPKYSLPELENGRKR | |
| | CAACGGCAGCATCCCCCACCAGATCCACCTG | | MLASAGELQKGNELALPSKYVNFLYLASHY | |
| | GGAGAGCTGCACGCCATTCTGCGGCGGCAGG | | EKLKGSPEDNEQKQLFVEQHKHYLDEIIEQ | |
| | AAGATTTTTACCCATTCCTGAAGGACAACCG | | ISEFSKRVILADANLDKVLSAYNKHRDKPI | |
| | GGAAAAGATCGAGAAGATCCTGACCTTCCGC | | REQAENIIHLFTLINLGAPAAFKYFDTTID | |
| | ATCCCCTACTACGTGGGCCCTCTGGCCAGGG | | RKRYTSTKEVLDATLIHQSITGLYETRIDL | |
| | GAAACAGCAGATTCGCCTGGATGACCAGAAA | | SQLGGDSGGSSGGSSGSETPGTSESATPES | |
| | GAGCGAGGAAACCATCACCCCCTGGAACTTC | | SGGSSGGSSDTSNLMEQILSSRNLNRAYLQ | |
| | GAGGAAGTGGTGGACAAGGGCGCTTCCGCCC | | VVPRKGABGVDGMKYTELKEHLAKNGETIK | |
| | AGAGCTTCATCGAGCGGATGACCAACTTCGA | | GQLRTRKYKPQPARRVEIPKPRGGVRNLGV | |
| | TAAGAACCTGCCCAACGAGAAGGTGCTGCCC | | PTVTDRFIQQAIAQVLTPIYEEQFHDHSYG | |
| | AAGCACAGCCTGCTGTACGAGTACTTCACCG | | FRPKRCAQQAILTALNIMNDGNDWIVDIDL | |
| | TGTATAACGAGCTGACCAAAGTGAAATACGT | | EKFFDTVNHDKLMTLIGRTIKDGDVISIVR | |
| | GACCGAGGGAATGAGAAAGCCCGCCTTCCTG | | KYLVSGIMIDDEYEDSIVGTPQGGRLSPLL | |
| | AGCGGCGAGCAGAAAAAGGCCATCGTGGACC | | ANIMLNELDKEMEKRGLNFVRYADDCIIMV | |
| | TGCTGTTCAAGACCAACCGGAAAGTGACCGT | | GSEMSANRVMRNISRFIEEKLGLKVNMTKS | |
| | GAAGCAGCTGAAAGAGGACTACTTCAAGAAA | | KVDRPSGLKYLGFGFYFDPRAHQFKAKPHA | |
| | ATCGAGTGCTTCGACTCCGTGGAAATCTCCG | | KSVAKFKKRMKELTCRSWGVSNSYKVEKLN | |
| | GCGTGGAAGATCGGTTCAACGCCTCCCTGGG | | QLIRGWINYFKIGSMKTLCKELDSRIRYRL | |
| | CACATACCACGATCTGCTGAAAATTATCAAG | | RMCIWKQWKTPQNQEKNIVKLGIDRNTARR | |
| | GACAAGGACTTCCTGGACAATGAGGAAAACG | | VAYTGKRIAYVCNKGAVNVAISNKRLASFG | |
| | AGGACATTCTGGAAGATATCGTGCTGACCCT | | LISMLDYYIEKCVTCSGGSKRTADGSEFEP | |
| | GACACTGTTTGAGGACAGAGAGATGATCGAG | | KKKRKVGSGATNFSLLKQAGDVEENPGPMV | |
| | GAACGGCTGAAAACCTATGCCCACCTGTTCG | | SKGEELFTGVVPILVELDGDVNGHKFSVSG | |
| | ACGACAAAGTGATGAAGCAGCTGAAGCGGCG | | EGEGDATYGKLTLKFICTTGKLPVPWPTLV | |
| | GAGATACACCGGCTGGGGCAGGCTGAGCCGG | | TTLTYGVQCFSRYPDHMKQHDFFKSAMPEG | |
| | AAGCTGATCAACGGCATCCGGGACAAGCAGT | | YVQERTIFFKDDGNYKTRAEVKFEGDTLVN | |
| | CCGGCAAGACAATCCTGGATTTCCTGAAGTC | | RIELKGIDFKEDGNILGHKLEYNYNSHNVY | |
| | CGACGGCTTCGCCAACAGAAACTTCATGCAG | | IMADKQKNGIKVNFKIRHNIEDGSVQLADH | |
| | CTGATCCACGACGACAGCCTGACCTTTAAAG | | YQQNTPIGDGPVLLPDNHYLSTQSALSKDP | |
| | AGGACATCCAGAAAGCCCAGGTGTCCGGCCA | | NEKRDHMVLLEFVTAAGITLGMDELYK* | |
| | GGGCGATAGCCTGCACGAGCACATTGCCAAT | | | |
| | CTGGCCGGCAGCCCCGCCATTAAGAAGGGCA | | | |
| | TCCTGCAGACAGTGAAGGTGGTGGACGAGCT | | | |
| | CGTGAAAGTGATGGGCCGGCACAAGCCCGAG | | | |
| | AACATCGTGATCGAAATGGCCAGAGAGAACC | | | |
| | AGACCACCCAGAAGGGACAGAAGAACAGCCG | | | |
| | CGAGAGAATGAAGCGGATCGAAGAGGGCATC | | | |
| | AAAGAGCTGGGCAGCCAGATCCTGAAAGAAC | | | |
| | ACCCCGTGGAAAACACCCAGCTGCAGAACGA | | | |
| | GAAGCTGTACCTGTACTACCTGCAGAATGGG | | | |
| | CGGGATATGTACGTGGACCAGGAACTGGACA | | | |
| | TCAACCGGCTGTCCGACTACGATGTGGACGC | | | |
| | TATCGTGCCTCAGAGCTTTCTGAAGGACGAC | | | |
| | TCCATCGACAACAAGGTGCTGACCAGAAGCG | | | |
| | ACAAGAACCGGGGCAAGAGCGACAACGTGCC | | | |
| | CTCCGAAGAGGTCGTGAAGAAGATGAAGAAC | | | |
| | TACTGGCGGCAGCTGCTGAACGCCAAGCTGA | | | |
| | TTACCCAGAGAAAGTTCGACAATCTGACCAA | | | |
| | GGCCGAGAGAGGCGGCCTGAGCGAACTGGAT | | | |
| | AAGGCCGGCTTCATCAAGAGACAGCTGGTGG | | | |
| | AAACCCGGCAGATCACAAAGCACGTGGCACA | | | |
| | GATCCTGGACTCCCGGATGAACACTAAGTAC | | | |
| | GACGAGAATGACAAGCTGATCCGGGAAGTGA | | | |
| | AAGTGATCACCCTGAAGTCCAAGCTGGTGTC | | | |
| | CGATTTCCGGAAGGATTTCCAGTTTTACAAA | | | |
| | GTGCGCGAGATCAACAACTACCACCACGCCC | | | |
| | ACGACGCCTACCTGAACGCCGTCGTGGGAAC | | | |
| | CGCCCTGATCAAAAAGTACCCTAAGCTGGAA | | | |
| | AGCGAGTTCGTGTACGGCGACTACAAGGTGT | | | |
| | ACGACGTGCGGAAGATGATCGCCAAGAGCGA | | | |
| | GCAGGAAATCGGCAAGGCTACCGCCAAGTAC | | | |
| | TTCTTCTACAGCAACATCATGAACTTTTTCA | | | |
| | AGACCGAGATTACCCTGGCCAACGGCGAGAT | | | |
| | CCGGAAGCGGCCTCTGATCGAGACAAACGGC | | | |
| | GAAACCGGGGAGATCGTGTGGGATAAGGGCC | | | |
| | GGGATTTTGCCACCGTGCGGAAAGTGCTGAG | | | |
| | CATGCCCCAAGTGAATATCGTGAAAAAGACC | | | |
| | GAGGTGCAGACAGGCGGCTTCAGCAAAGAGT | | | |
| | CTATCCTGCCCAAGAGGAACAGCGATAAGCT | | | |
| | GATCGCCAGAAAGAAGGACTGGGACCCTAAG | | | |
| | AAGTACGGCGGCTTCGACAGCCCCACCGTGG | | | |
| | CCTATTCTGTGCTGGTGGTGGCCAAAGTGGA | | | |
| | AAAGGGCAAGTCCAAGAAACTGAAGAGTGTG | | | |
| | AAAGAGCTGCTGGGGATCACCATCATGGAAA | | | |
| | GAAGCAGCTTCGAGAAGAATCCCATCGACTT | | | |
| | TCTGGAAGCCAAGGGCTACAAAGAAGTGAZA | | | |
| | AAGGACCTGATCATCAAGCTGCCTAAGTACT | | | |
| | CCCTGTTCGAGCTGGAAAACGGCCGGAAGAG | | | |
| | AATGCTGGCCTCTGCCGGCGAACTGCAGAAG | | | |
| | GGAAACGAACTGGCCCTGCCCTCCAAATATG | | | |
| | TGAACTTCCTGTACCTGGCCAGCCACTATGA | | | |
| | GAAGCTGAAGGGCTCCCCCGAGGATAATGAG | | | |
| | CAGAAACAGCTGTTTGTGGAACAGCACAAGC | | | |
| | ACTACCTGGACGAGATCATCGAGCAGATCAG | | | |
| | CGAGTTCTCCAAGAGAGTGATCCTGGCCGAC | | | |
| | GCTAATCTGGACAAAGTGCTGTCCGCCTACA | | | |
| | ACAAGCACCGGGATAAGCCCATCAGAGAGCA | | | |
| | GGCCGAGAATATCATCCACCTGTTTACCCTG | | | |
| | ACCAATCTGGGAGCCCCTGCCGCCTTCAAGT | | | |
| | ACTTTGACACCACCATCGACCGGAAGAGGTA | | | |
| | CACCAGCACCAAAGAGGTGCTGGACGCCACC | | | |
| | CTGATCCACCAGAGCATCACCGGCCTGTACG | | | |
| | AGACACGGATCGACCTGTCTCAGCTGGGAGG | | | |
| | TGACTCTGGAGGATCTAGCGGAGGATCCTCT | | | |
| | GGCAGCGAGACACCAGGAACAAGCGAGTCAG | | | |
| | CAACACCAGAGAGCAGTGGCGGCAGCAGCGG | | | |
| | CGGCAGCAGCGACACCAGCAATCTGATGGAA | | | |
| | CAGATCCTGAGCAGCCGGAACCTGAACCGGG | | | |
| | CCTACCTGCAGGTGGTGAGACGGAAAGGCGC | | | |
| | TGAAGGCGTTGATGGCATGAAGTACACCGAG | | | |
| | CTGAAGGAGCATCTGGCCAAGAACGGCGAGA | | | |
| | CAATCAAGGGCCAGCTGAGAACCAGAAAGTA | | | |
| | TAAGCCTCAGCCAGCTAGACGGGTGGAAATC | | | |
| | CCCAAGCCCCGGGGCGGAGTGCGGAACCTGG | | | |
| | GAGTGCCAACAGTCACAGACCGGTTCATCCA | | | |
| | GCAGGCTATCGCCCAAGTGCTGACCCCTATC | | | |
| | TACGAGGAACAGTTTCACGACCACTCTTACG | | | |
| | GCTTCCGGCCCAAGAGATGCGCCCAGCAAGC | | | |
| | CATCCTGACAGCCCTGAACATCATGAACGAT | | | |
| | GGTAATGACTGGATCGTGGACATCGACCTGG | | | |
| | AAAAGTTTTTCGATACCGTGAATCACGATAA | | | |
| | GCTGATGACGCTGATTGGCAGAACCATCAAG | | | |
| | GACGGCGACGTGATCTCTATTGTGCGCAAGT | | | |
| | ACCTCGTGTCCGGCATCATGATCGATGACGA | | | |
| | GTACGAAGATAGCATCGTGGGAACACCTCAG | | | |
| | GGCGGCCGGCTGTCTCCTCTGCTGGCCAACA | | | |
| | TCATGCTGAACGAGCTGGATAAGGAGATGGA | | | |
| | AAAAAGGGGCCTGAACTTCGTGCGGTACGCC | | | |
| | GACGACTGCATCATCATGGTCGGCTCCGAGA | | | |
| | TGAGCGCCAACAGAGTCATGCGGAACATCAG | | | |
| | CAGATTCATCGAAGAGAAGCTGGGCCTGAAA | | | |
| | GTGAACATGACCAAGTCCAAGGTGGACAGAC | | | |
| | CTAGCGGACTGAAGTACTTGGGCTTTGGCTT | | | |
| | CTACTTCGACCCCAGAGCCCACCAGTTCAAG | | | |
| | GCCAAGCCTCACGCCAAGAGCGTGGCTAAGT | | | |
| | TCAAAAAGAGAATGAAAGAGCTGACCTGTAG | | | |
| | AAGCTGGGGCGTGTCTAACAGCTACAAGGTG | | | |
| | GAAAAACTGAATCAACTGATCAGAGGCTGGA | | | |
| | TCAACTACTTCAAGATCGGCAGCATGAAGAC | | | |
| | CCTGTGTAAAGAGCTGGACAGCAGAATCAGG | | | |
| | TACAGACTGCGGATGTGCATCTGGAAGCAGT | | | |
| | GGAAAACCCCTCAGAACCAGGAGAAAAACCT | | | |
| | GGTCAAGCTTGGAATTGACAGAAATACCGCC | | | |
| | AGAAGAGTGGCCTATACAGGCAAGCGAATCG | | | |
| | CCTACGTGTGCAACAAGGGCGCCGTGAACGT | | | |
| | GGCTATCAGCAACAAGCGGCTGGCCAGCTTC | | | |
| | GGCCTGATCTCTATGCTGGACTACTACATCG | | | |
| | AGAAGTGCGTGACCTGCTCTGGCGGCTCAAA | | | |
| | AAGAACCGCCGACGGCAGCGAATTCGAGCCC | | | |
| | AAGAAGAAGAGGAAAGTCGGAAGCGGAGCTA | | | |
| | CTAACTTCAGCCTGCTGAAGCAGGCTGGAGA | | | |
| | CGTGGAGGAGAACCCTGGACCTATGGTGAGC | | | |
| | AAGGGCGAGGAGCTGTTCACCGGGGTGGTGC | | | |
| | CCATCCTGGTCGAGCTGGACGGCGACGTAZA | | | |
| | CGGCCACAAGTTCAGCGTGTCCGGCGAGGGC | | | |
| | GAGGGCGATGCCACCTACGGCAAGCTGACCC | | | |
| | TGAAGTTCATCTGCACCACCGGCAAGCTGCC | | | |
| | CGTGCCCTGGCCCACCCTCGTGACCACCCTG | | | |
| | ACCTATGGAGTGCAGTGCTTCAGCCGCTACC | | | |
| | CCGACCACATGAAGCAGCACGACTTCTTCAA | | | |
| | GTCCGCCATGCCCGAAGGCTACGTCCAGGAG | | | |
| | CGCACCATCTTCTTCAAGGACGACGGCAACT | | | |
| | ACAAGACCCGCGCCGAGGTGAAGTTCGAGGG | | | |
| | CGACACCCTGGTGAACCGCATCGAGCTGAAG | | | |
| | GGCATCGACTTCAAGGAGGACGGCAACATCC | | | |
| | TGGGGCACAAGCTGGAGTACAACTACAACAG | | | |
| | CCACAACGTCTATATCATGGCCGACAAGCAG | | | |
| | AAGAACGGCATCAAGGTGAACTTCAAGATCC | | | |
| | GCCACAACATCGAGGACGGCAGCGTGCAGCT | | | |
| | CGCCGACCACTACCAGCAGAACACCCCCATC | | | |
| | GGCGACGGCCCCGTGCTGCTGCCCGACAACC | | | |
| | ACTACCTGAGCACCCAGTCCGCCCTGAGCAA | | | |
| | AGACCCCAACGAGAAGCGCGATCACATGGTC | | | |
| | CTGCTGGAGTTCGTGACCGCCGCCGGGATCA | | | |
| | CTCTCGGCATGGACGAGCTGTACAAGTAA | | | |
| bpNLS- | ATGAAACGGACAGCCGACGGAAGCGAGTTCG | 110 | MKRTADGSEFESPKKKRKVDTSNLMEQILS | 111 |
| Marathon | AGTCACCAAAGAAGAAGCGGAAAGTCGACAC | | SRNLNRAYLQVVRNKGAEGVDGMKYTELKE | |
| (D14R- | CAGCAATCTGATGGAACAGATCCTGAGCAGC | | HLAKNGETIKGQLRTRKYKPQPARRVEIPK | |
| D74R- | CGGAACCTGAACCGGGCCTACCTGCAGGTGG | | PRGGVRNLGVPTVTDRFIQQAIAQVLTPIY | |
| N116K- | TGAGAAATAAAGGCGCTGAAGGCGTTGATGG | | ERQFHDHSYGFRPKRCAQQAILTALNIMND | |
| N197R) | CATGAAGTACACCGAGCTGAAGGAGCATCTG | | GNDWIVDIDLEKFFDTVNHDKLMTLIGRTI | |
| RT-4 AA | GCCAAGAACGGCGAGACAATCAAGGGCCAGC | | KDGDVISIVRKYLVSGIMIDDEYEDSIVGT | |
| linker- | TGAGAACCAGAAAGTATAAGCCTCAGCCAGC | | PQGGRLSPLLANIMLNELDKEMEKRGLNFV | |
| bpNLS | TAGACGGGTGGAAATCCCCAAGCCCCGGGGC | | RYADDCIIMVGSEMSANRVMRNISRFIEEK | |
| | GGAGTGCGGAACCTGGGAGTGCCAACAGTCA | | LGLKVNMTKSKVDRPSGLKYLGFGFYFDPR | |
| | CAGACCGGTTCATCCAGCAGGCTATCGCCCA | | AHQFKAKPHAKSVAKFKKRMKELTCRSWGV | |
| | AGTGCTGACCCCTATCTACGAGGAACAGTTT | | SNSYKVEKLNQLIRGWINYFKIGSMKILCK | |
| | CACGACCACTCTTACGGCTTCCGGCCCAAGA | | ELDSRIRYRLRMCIWKQWKTPQNQEKNLVK | |
| | GATGCGCCCAGCAAGCCATCCTGACAGCCCT | | LGIDRNTARRVAYTGKRIAYVCNKGAVNVA | |
| | GAACATCATGAACGATGGTAATGACTGGATC | | ISNKRLASFGLISMLDYYIEKCVTCSGGSK | |
| | GTGGACATCGACCTGGAAAAGTTTTTCGATA | | RTADGSEFEPKKKRKV* | |
| | CCGTGAATCACGATAAGCTGATGACGCTGAT | | | |
| | TGGCAGAACCATCAAGGACGGCGACGTGATC | | | |
| | TCTATTGTGCGCAAGTACCTCGTGTCCGGCA | | | |
| | TCATGATCGATGACGAGTACGAAGATAGCAT | | | |
| | CGTGGGAACACCTCAGGGCGGCCGGCTGTCT | | | |
| | CCTCTGCTGGCCAACATCATGCTGAACGAGC | | | |
| | TGGATAAGGAGATGGAAAAAAGGGGCCTGAA | | | |
| | CTTCGTGCGGTACGCCGACGACTGCATCATC | | | |
| | ATGGTCGGCTCCGAGATGAGCGCCAACAGAG | | | |
| | TCATGCGGAACATCAGCAGATTCATCGAAGA | | | |
| | GAAGCTGGGCCTGAAAGTGAACATGACCAAG | | | |
| | TCCAAGGTGGACAGACCTAGCGGACTGAAGT | | | |
| | ACTTGGGCTTTGGCTTCTACTTCGACCCCAG | | | |
| | AGCCCACCAGTTCAAGGCCAAGCCTCACGCC | | | |
| | AAGAGCGTGGCTAAGTTCAAAAAGAGAATGA | | | |
| | AAGAGCTGACCTGTAGAAGCTGGGGCGTGTC | | | |
| | TAACAGCTACAAGGTGGAAAAACTGAATCAA | | | |
| | CTGATCAGAGGCTGGATCAACTACTTCAAGA | | | |
| | TCGGCAGCATGAAGACCCTGTGTAAAGAGCT | | | |
| | GGACAGCAGAATCAGGTACAGACTGCGGATG | | | |
| | TGCATCTGGAAGCAGTGGAAAACCCCTCAGA | | | |
| | ACCAGGAGAAAAACCTGGTCAAGCTTGGAAT | | | |
| | TGACAGAAATACCGCCAGAAGAGTGGCCTAT | | | |
| | ACAGGCAAGCGAATCGCCTACGTGTGCAACA | | | |
| | AGGGCGCCGTGAACGTGGCTATCAGCAACAA | | | |
| | GCGGCTGGCCAGCTTCGGCCTGATCTCTATG | | | |
| | CTGGACTACTACATCGAGAAGTGCGTGACCT | | | |
| | GCTCTGGCGGCTCAAAAAGAACCGCCGACGG | | | |
| | CAGCGAATTCGAGCCCAAGAAGAAGAGGAAA | | | |
| | GTCTAA | | | |
| bpNLS- | ATGAAACGGACAGCCGACGGAAGCGAGTTCG | 112 | MKRTADGSEFESPKKKRKVDKKYSIGLDIG | 113 |
| nCas9 | AGTCACCAAAGAAGAAGCGGAAAGTCGACAA | | TNSVGWAVITDEYKVPSKKFKVLGNTDRHS | |
| (N)-N | GAAGTACAGCATCGGCCTGGACATCGGCACC | | IKKNLIGALLFDSGETAEATRLKRTARRRY | |
| intein | AACTCTGTGGGCTGGGCCGTGATCACCGACG | | TRRKNRICYLQEIFSNEMAKVDDSFFHRLE | |
| | AGTACAAGGTGCCCAGCAAGAAATTCAAGGT | | ESFLVEEDKKHERHPIFGNIVDEVAYHEKY | |
| | GCTGGGCAACACCGACCGGCACAGCATCAAG | | PTIYHERKKLVDSTDKADLRLIYLALAHMI | |
| | AAGAACCTGATCGGAGCCCTGCTGTTCGACA | | KFRGHFLIEGDLNPDNSDVDKLFIQLVQTY | |
| | GCGGCGAAACAGCCGAGGCCACCCGGCTGAA | | NQLFEENPINASGVDAKAILSARLSKSRRL | |
| | GAGAACCGCCAGAAGAAGATACACCAGACGG | | ENLIAQLPGEKKNGLEGNLIALSLGLIPNF | |
| | AAGAACCGGATCTGCTATCTGCAAGAGATCT | | KSNEDLAEDAKLQLSKDTYDDDLDNLLAQI | |
| | TCAGCAACGAGATGGCCAAGGTGGACGACAG | | GDQYADLFLAAKNLSDAILLSDILRVNTEI | |
| | CTTCTTCCACAGACTGGAAGAGTCCTTCCTG | | TKAPLSASMIKRYDEHHQDLTLLKALVRQQ | |
| | GTGGAAGAGGATAAGAAGCACGAGCGGCACC | | LPEKYKEIFFDQSKNGYAGYIDGGASQEEF | |
| | CCATCTTCGGCAACATCGTGGACGAGGTGGC | | YKFIKPILEKMDGTEELLVKLNREDLLRKQ | |
| | CTACCACGAGAAGTACCCCACCATCTACCAC | | RTFDNGSIPHQIHLGELHAILRRQEDFYPF | |
| | CTGAGAAAGAAACTGGTGGACAGCACCGACA | | LKDNREKIEKILTFRIPYYVGPLARGNSRF | |
| | AGGCCGACCTGCGGCTGATCTATCTGGCCCT | | AWMTRKSEETITPWNFEEVVDKGASAQSFI | |
| | GGCCCACATGATCAAGTTCCGGGGCCACTTC | | ERMTNFDKNLPNEKVLPKHSLLYEYFTVYN | |
| | CTGATCGAGGGCGACCTGAACCCCGACAACA | | ELTKVKYVTEGMRKPAFLSGEQKKAIVDLL | |
| | GCGACGTGGACAAGCTGTTCATCCAGCTGGT | | FKTNRKVTVKQLKEDYFKKIECFDSVEISG | |
| | GCAGACCTACAACCAGCTGTTCGAGGAAAAC | | VEDRFNASLGTYHDLLKIIKDKDELDNEEN | |
| | CCCATCAACGCCAGCGGCGTGGACGCCAAGG | | EDILEDIVLTLILFEDREMIEERLKTYAHL | |
| | CCATCCTGTCTGCCAGACTGAGCAAGAGCAG | | FDDKVMKQLKRRRYTGWGRLSRKLINGIRD | |
| | ACGGCTGGAAAATCTGATCGCCCAGCTGCCC | | KQSGKTILDFLKSDGFANRNFMQLIHDDSL | |
| | GGCGAGAAGAAGAATGGCCTGTTCGGAAACC | | TEKEDIQKAQVCLSYETEILTVEYGLLPIG | |
| | TGATTGCCCTGAGCCTGGGCCTGACCCCCAA | | KIVEKRIECTVYSVDNNGNIYTQPVAQWHD | |
| | CTTCAAGAGCAACTTCGACCTGGCCGAGGAT | | RGEQEVFEYCLEDGSLIRATKDHKFMTVDG | |
| | GCCAAACTGCAGCTGAGCAAGGACACCTACG | | QMLPIDEIFERELDLMRVDNLPN* | |
| | ACGACGACCTGGACAACCTGCTGGCCCAGAT | | | |
| | CGGCGACCAGTACGCCGACCTGTTTCTGGCC | | | |
| | GCCAAGAACCTGTCCGACGCCATCCTGCTGA | | | |
| | GCGACATCCTGAGAGTGAACACCGAGATCAC | | | |
| | CAAGGCCCCCCTGAGCGCCTCTATGATCAAG | | | |
| | AGATACGACGAGCACCACCAGGACCTGACCC | | | |
| | TGCTGAAAGCTCTCGTGCGGCAGCAGCTGCC | | | |
| | TGAGAAGTACAAAGAGATTTTCTTCGACCAG | | | |
| | AGCAAGAACGGCTACGCCGGCTACATTGACG | | | |
| | GCGGAGCCAGCCAGGAAGAGTTCTACAAGTT | | | |
| | CATCAAGCCCATCCTGGAAAAGATGGACGGC | | | |
| | ACCGAGGAACTGCTCGTGAAGCTGAACAGAG | | | |
| | AGGACCTGCTGCGGAAGCAGCGGACCTTCGA | | | |
| | CAACGGCAGCATCCCCCACCAGATCCACCTG | | | |
| | GGAGAGCTGCACGCCATTCTGCGGCGGCAGG | | | |
| | AAGATTTTTACCCATTCCTGAAGGACAACCG | | | |
| | GGAAAAGATCGAGAAGATCCTGACCTTCCGC | | | |
| | ATCCCCTACTACGTGGGCCCTCTGGCCAGGG | | | |
| | GAAACAGCAGATTCGCCTGGATGACCAGAAA | | | |
| | GAGCGAGGAAACCATCACCCCCTGGAACTTC | | | |
| | GAGGAAGTGGTGGACAAGGGCGCTTCCGCCC | | | |
| | AGAGCTTCATCGAGCGGATGACCAACTTCGA | | | |
| | TAAGAACCTGCCCAACGAGAAGGTGCTGCCC | | | |
| | AAGCACAGCCTGCTGTACGAGTACTTCACCG | | | |
| | TGTATAACGAGCTGACCAAAGTGAAATACGT | | | |
| | GACCGAGGGAATGAGAAAGCCCGCCTTCCTG | | | |
| | AGCGGCGAGCAGAAAAAGGCCATCGTGGACC | | | |
| | TGCTGTTCAAGACCAACCGGAAAGTGACCGT | | | |
| | GAAGCAGCTGAAAGAGGACTACTTCAAGAAA | | | |
| | ATCGAGTGCTTCGACTCCGTGGAAATCTCCG | | | |
| | GCGTGGAAGATCGGTTCAACGCCTCCCTGGG | | | |
| | CACATACCACGATCTGCTGAAAATTATCAAG | | | |
| | GACAAGGACTTCCTGGACAATGAGGAAAACG | | | |
| | AGGACATTCTGGAAGATATCGTGCTGACCCT | | | |
| | GACACTGTTTGAGGACAGAGAGATGATCGAG | | | |
| | GAACGGCTGAAAACCTATGCCCACCTGTTCG | | | |
| | ACGACAAAGTGATGAAGCAGCTGAAGCGGCG | | | |
| | GAGATACACCGGCTGGGGCAGGCTGAGCCGG | | | |
| | AAGCTGATCAACGGCATCCGGGACAAGCAGT | | | |
| | CCGGCAAGACAATCCTGGATTTCCTGAAGTC | | | |
| | CGACGGCTTCGCCAACAGAAACTTCATGCAG | | | |
| | CTGATCCACGACGACAGCCTGACCTTTAAAG | | | |
| | AGGACATCCAGAAAGCCCAGGTGTGCCTGTC | | | |
| | CTACGAGACAGAGATCCTGACAGTGGAGTAT | | | |
| | GGCCTGCTGCCAATCGGCAAGATCGTGGAGA | | | |
| | AGAGGATCGAGTGTACCGTGTACTCTGTGGA | | | |
| | TAACAATGGCAACATCTATACACAGCCCGTG | | | |
| | GCACAGTGGCACGATAGGGGAGAGCAGGAGG | | | |
| | TGTTCGAGTATTGCCTGGAGGACGGCAGCCT | | | |
| | GATCAGGGCAACCAAGGACCACAAGTTCATG | | | |
| | ACAGTGGATGGCCAGATGCTGCCCATCGACG | | | |
| | AGATTTTCGAGCGGGAGCTGGACCTGATGAG | | | |
| | AGTGGATAACCTGCCTAATTAA | | | |
| C | ATGATCAAGATTGCTACACGGAAATACCTGG | 114 | MIKIATRKYLGKQNVYDIGVERDHNFALKN | 115 |
| intein- | GAAAGCAGAACGTGTACGACATCGGCGTGGA | | GFIASNSGQGDSLHEHIANLAGSPAIKKGI | |
| nCas9 | GCGGGATCACAACTTCGCCCTGAAGAATGGC | | LQTVKVVDELVKVMGRHKPENIVIEMAREN | |
| (C)- | TTTATCGCCAGCAATTCCGGCCAGGGCGATA | | QTTQKGQKNSRERMKRIEEGIKELGSQILK | |
| XTEN- | GCCTGCACGAGCACATTGCCAATCTGGCCGG | | EHPVENTQLQNEKLYLYYLQNGRDMYVDQE | |
| MMLV | CAGCCCCGCCATTAAGAAGGGCATCCTGCAG | | LDINRLSDYDVDAIVPQSFLKDDSIDNKVL | |
| RT- | ACAGTGAAGGTGGTGGACGAGCTCGTGAAAG | | TRSDKNRGKSDNVPSEEVVKKMKNYWRQLL | |
| bpNLS | TGATGGGCCGGCACAAGCCCGAGAACATCGT | | NAKLITQRKEDNITKAERGGLSELDKAGFI | |
| | GATCGAAATGGCCAGAGAGAACCAGACCACC | | KRQLVETRQITKHVAQILDSRMNTKYDEND | |
| | CAGAAGGGACAGAAGAACAGCCGCGAGAGAA | | KLIREVKVITLKSKLVSDFRKDFQFYKVRE | |
| | TGAAGCGGATCGAAGAGGGCATCAAAGAGCT | | INNYHHAHDAYLNAVVGTALIKKYPKLESE | |
| | GGGCAGCCAGATCCTGAAAGAACACCCCGTG | | FVYGDYKVYDVRKMIAKSEQEIGKATAKYF | |
| | GAAAACACCCAGCTGCAGAACGAGAAGCTGT | | FYSNIMNFPKTEITLANGEIRKRPLIETNG | |
| | ACCTGTACTACCTGCAGAATGGGCGGGATAT | | ETGEIVWDKGRDFATVRKVLSMPQVNIVKK | |
| | GTACGTGGACCAGGAACTGGACATCAACCGG | | TEVQTGGFSKESILPKRNSDKLIARKKDWD | |
| | CTGTCCGACTACGATGTGGACGCTATCGTGC | | PKKYGGFDSPTVAYSVLVVAKVEKGKSKKL | |
| | CTCAGAGCTTTCTGAAGGACGACTCCATCGA | | KSVKELLGITIMERSSFEKNPIDFLEAKGY | |
| | CAACAAGGTGCTGACCAGAAGCGACAAGAAC | | KEVKKDLIIKLPKYSLFELENGRKRMLASA | |
| | CGGGGCAAGAGCGACAACGTGCCCTCCGAAG | | GELQKGNELALPSKYVNFLYLASHYEKLKG | |
| | AGGTCGTGAAGAAGATGAAGAACTACTGGCG | | SPEDNEQKQLFVEQHKHYLDEIIEQISEFS | |
| | GCAGCTGCTGAACGCCAAGCTGATTACCCAG | | KRVILADANLDKVLSAYNKHRDKPIREQAE | |
| | AGAAAGTTCGACAATCTGACCAAGGCCGAGA | | NIIHLFTLINIGAPAAFKYFDTTIDRKRYT | |
| | GAGGCGGCCTGAGCGAACTGGATAAGGCCGG | | STKEVLDATLIHQSITGLYETRIDLSQLGG | |
| | CTTCATCAAGAGACAGCTGGTGGAAACCCGG | | DSGGSSGGSSGSETPGTSESATPESSGGSS | |
| | CAGATCACAAAGCACGTGGCACAGATCCTGG | | GGSSTLNIEDEYRLHETSKEPDVSLGSTWL | |
| | ACTCCCGGATGAACACTAAGTACGACGAGAA | | SDFPQAWAETGGMGLAVRQAPLIIPLKATS | |
| | TGACAAGCTGATCCGGGAAGTGAAAGTGATC | | TPVSIKQYPMSQEARLGIKPHIQRLLDQGI | |
| | ACCCTGAAGTCCAAGCTGGTGTCCGATTTCC | | LVPCQSPWNTPLLPVKKPGTNDYRPVQDLR | |
| | GGAAGGATTTCCAGTTTTACAAAGTGCGCGA | | EVNKRVEDIHPTVPNPYNLLSGLPPSHQWY | |
| | GATCAACAACTACCACCACGCCCACGACGCC | | TVLDLKDAFFCLRLHPTSQPLFAFEWRDPE | |
| | TACCTGAACGCCGTCGTGGGAACCGCCCTGA | | MGISGQLTWTRLPQGFKNSPTLFNEALHRD | |
| | TCAAAAAGTACCCTAAGCTGGAAAGCGAGTT | | LADFRIQHPDLILLQYVDDLLLAATSELDC | |
| | CGTGTACGGCGACTACAAGGTGTACGACGTG | | QQGTRALLQTLGNLGYRASAKKAQICQKQV | |
| | CGGAAGATGATCGCCAAGAGCGAGCAGGAAA | | KYLGYLLKEGQRWLTEARKETVMGQPTPKT | |
| | TCGGCAAGGCTACCGCCAAGTACTTCTTCTA | | PRQLREFLGKAGFCRLFIPGFAEMAAPLYP | |
| | CAGCAACATCATGAACTTTTTCAAGACCGAG | | LTKPGTLFNWGPDQQKAYQEIKQALLTAPA | |
| | ATTACCCTGGCCAACGGCGAGATCCGGAAGC | | LGLPDLTKPFELFVDEKQGYAKGVLTQKLG | |
| | GGCCTCTGATCGAGACAAACGGCGAAACCGG | | PWRRPVAYLSKKLDPVAAGWPPCLRMVAAI | |
| | GGAGATCGTGTGGGATAAGGGCCGGGATTTT | | AVLTKDAGKLIMGQPLVILAPHAVEALVKQ | |
| | GCCACCGTGCGGAAAGTGCTGAGCATGCCCC | | PPDRWLSNARMTHYQALLLDTDRVQFGPVV | |
| | AAGTGAATATCGTGAAAAAGACCGAGGTGCA | | ALNPATLLPLPEEGLQHNCLDILAEAHGTR | |
| | GACAGGCGGCTTCAGCAAAGAGTCTATCCTG | | PDLTDQPLPDADHTWYTDGSSLLQEGQRKA | |
| | CCCAAGAGGAACAGCGATAAGCTGATCGCCA | | GAAVITETEVIWAKALPAGTSAQRAELIAL | |
| | GAAAGAAGGACTGGGACCCTAAGAAGTACGG | | TQALKMAEGKKLNVYTDSRYAFATAHINGE | |
| | CGGCTTCGACAGCCCCACCGTGGCCTATTCT | | IYRRRGWLTSEGKEIKNKDEILALLKALFL | |
| | GTGCTGGTGGTGGCCAAAGTGGAAAAGGGCA | | PKRLSIIHCPGHQKGHSAEARGNRMADQAA | |
| | AGTCCAAGAAACTGAAGAGTGTGAAAGAGCT | | RKAAITETPDTSTLLIENSSPSGGSKRTAD | |
| | GCTGGGGATCACCATCATGGAAAGAAGCAGC | | GSEFEPKKKRKV* | |
| | TTCGAGAAGAATCCCATCGACTTTCTGGAAG | | | |
| | CCAAGGGCTACAAAGAAGTGAAAAAGGACCT | | | |
| | GATCATCAAGCTGCCTAAGTACTCCCTGTTC | | | |
| | GAGCTGGAAAACGGCCGGAAGAGAATGCTGG | | | |
| | CCTCTGCCGGCGAACTGCAGAAGGGAAACGA | | | |
| | ACTGGCCCTGCCCTCCAAATATGTGAACTTC | | | |
| | CTGTACCTGGCCAGCCACTATGAGAAGCTGA | | | |
| | AGGGCTCCCCCGAGGATAATGAGCAGAAACA | | | |
| | GCTGTTTGTGGAACAGCACAAGCACTACCTG | | | |
| | GACGAGATCATCGAGCAGATCAGCGAGTTCT | | | |
| | CCAAGAGAGTGATCCTGGCCGACGCTAATCT | | | |
| | GGACAAAGTGCTGTCCGCCTACAACAAGCAC | | | |
| | CGGGATAAGCCCATCAGAGAGCAGGCCGAGA | | | |
| | ATATCATCCACCTGTTTACCCTGACCAATCT | | | |
| | GGGAGCCCCTGCCGCCTTCAAGTACTTTGAC | | | |
| | ACCACCATCGACCGGAAGAGGTACACCAGCA | | | |
| | CCAAAGAGGTGCTGGACGCCACCCTGATCCA | | | |
| | CCAGAGCATCACCGGCCTGTACGAGACACGG | | | |
| | ATCGACCTGTCTCAGCTGGGAGGTGACTCTG | | | |
| | GAGGATCTAGCGGAGGATCCTCTGGCAGCGA | | | |
| | GACACCAGGAACAAGCGAGTCAGCAACACCA | | | |
| | GAGAGCAGTGGCGGCAGCAGCGGCGGCAGCA | | | |
| | GCACCCTAAATATAGAAGATGAGTATCGGCT | | | |
| | ACATGAGACCTCAAAAGAGCCAGATGTTTCT | | | |
| | CTAGGGTCCACATGGCTGTCTGATTTTCCTC | | | |
| | AGGCCTGGGCGGAAACCGGGGGCATGGGACT | | | |
| | GGCAGTTCGCCAAGCTCCTCTGATCATACCT | | | |
| | CTGAAAGCAACCTCTACCCCCGTGTCCATAA | | | |
| | AACAATACCCCATGTCACAAGAAGCCAGACT | | | |
| | GGGGATCAAGCCCCACATACAGAGACTGTTG | | | |
| | GACCAGGGAATACTGGTACCCTGCCAGTCCC | | | |
| | CCTGGAACACGCCCCTGCTACCCGTTAAGAA | | | |
| | ACCAGGGACTAATGATTATAGGCCTGTCCAG | | | |
| | GATCTGAGAGAAGTCAACAAGCGGGTGGAAG | | | |
| | ACATCCACCCCACCGTGCCCAACCCTTACAA | | | |
| | CCTCTTGAGCGGGCTCCCACCGTCCCACCAG | | | |
| | TGGTACACTGTGCTTGATTTAAAGGATGCCT | | | |
| | TTTTCTGCCTGAGACTCCACCCCACCAGTCA | | | |
| | GCCTCTCTTCGCCTTTGAGTGGAGAGATCCA | | | |
| | GAGATGGGAATCTCAGGACAATTGACCTGGA | | | |
| | CCAGACTCCCACAGGGTTTCAAAAACAGTCC | | | |
| | CACCCTGTTTAATGAGGCACTGCACAGAGAC | | | |
| | CTAGCAGACTTCCGGATCCAGCACCCAGACT | | | |
| | TGATCCTGCTACAGTACGTGGATGACTTACT | | | |
| | GCTGGCCGCCACTTCTGAGCTAGACTGCCAA | | | |
| | CAAGGTACTCGGGCCCTGTTACAAACCCTAG | | | |
| | GGAACCTCGGGTATCGGGCCTCGGCCAAGAA | | | |
| | AGCCCAAATTTGCCAGAAACAGGTCAAGTAT | | | |
| | CTGGGGTATCTTCTAAAAGAGGGTCAGAGAT | | | |
| | GGCTGACTGAGGCCAGAAAAGAGACTGTGAT | | | |
| | GGGGCAGCCTACTCCGAAGACCCCTCGACAA | | | |
| | CTAAGGGAGTTCCTAGGGAAGGCAGGCTTCT | | | |
| | GTCGCCTCTTCATCCCTGGGTTTGCAGAAAT | | | |
| | GGCAGCCCCCCTGTACCCTCTCACCAAACCG | | | |
| | GGGACTCTGTTTAATTGGGGCCCAGACCAAC | | | |
| | AAAAGGCCTATCAAGAAATCAAGCAAGCTCT | | | |
| | TCTAACTGCCCCAGCCCTGGGGTTGCCAGAT | | | |
| | TTGACTAAGCCCTTTGAACTCTTTGTCGACG | | | |
| | AGAAGCAGGGCTACGCCAAAGGTGTCCTAAC | | | |
| | GCAAAAACTGGGACCTTGGCGTCGGCCGGTG | | | |
| | GCCTACCTGTCCAAAAAGCTAGACCCAGTAG | | | |
| | CAGCTGGGTGGCCCCCTTGCCTACGGATGGT | | | |
| | AGCAGCCATTGCCGTACTGACAAAGGATGCA | | | |
| | GGCAAGCTAACCATGGGACAGCCACTAGTCA | | | |
| | TTCTGGCCCCCCATGCAGTAGAGGCACTAGT | | | |
| | CAAACAACCCCCCGACCGCTGGCTTTCCAAC | | | |
| | GCCCGGATGACTCACTATCAGGCCTTGCTTT | | | |
| | TGGACACGGACCGGGTCCAGTTCGGACCGGT | | | |
| | GGTAGCCCTGAACCCGGCTACGCTGCTCCCA | | | |
| | CTGCCTGAGGAAGGGCTGCAACACAACTGCC | | | |
| | TTGATATCCTGGCCGAAGCCCACGGAACCCG | | | |
| | ACCCGACCTAACGGACCAGCCGCTCCCAGAC | | | |
| | GCCGACCACACCTGGTACACGGATGGAAGCA | | | |
| | GTCTCTTACAAGAGGGACAGCGTAÄGGCGGG | | | |
| | AGCTGCGGTGACCACCGAGACCGAGGTAATC | | | |
| | TGGGCTAAAGCCCTGCCAGCCGGGACATCCG | | | |
| | CTCAGCGGGCTGAACTGATAGCACTCACCCA | | | |
| | GGCCCTAAAGATGGCAGAAGGTAAGAAGCTA | | | |
| | AATGTTTATACTGATAGCCGTTATGCTTTTG | | | |
| | CTACTGCCCATATCCATGGAGAAATATACAG | | | |
| | AAGGCGTGGGTGGCTCACATCAGAAGGCAAA | | | |
| | GAGATCAAAAATAAAGACGAGATCTTGGCCC | | | |
| | TACTAAAAGCCCTCTTTCTGCCCAAAAGACT | | | |
| | TAGCATAATCCATTGTCCAGGACATCAAAAG | | | |
| | GGACACAGCGCCGAGGCTAGAGGCAACCCGA | | | |
| | TGGCTGACCAAGCGGCCCGAAAGGCAGCCAT | | | |
| | CACAGAGACTCCAGACACCTCTACCCTCCTC | | | |
| | ATAGAAAATTCATCACCCTCTGGCGGCTCAA | | | |
| | AAAGAACCGCCGACGGCAGCGAATTCGAGCC | | | |
| | CAAGAAGAAGAGGAAAGTCTAA | | | |
| bpNLS- | ATGAAACGGACAGCCGACGGAAGCGAGTTCG | 116 | MKRTADGSEFESPKKKRKVDKKYSIGLDIG | 117 |
| nCas9 | AGTCACCAAAGAAGAAGCGGAAAGTCGACAA | | TNSVGWAVITDEYKVPSKKFKVLGNTDRHS | |
| (H840A)- | GAAGTACAGCATCGGCCTGGACATCGGCACC | | IKKNLIGALLFDSGETAEATRLKRTARRRY | |
| XTEN- | AACTCTGTGGGCTGGGCCGTGATCACCGACG | | TRRKNRICYLQEIFSNEMAKVDDSFFHRLE | |
| Marathon | AGTACAAGGTGCCCAGCAAGAAATTCAAGGT | | ESFLVEEDKKHERHPIFGNIVDEVAYHEKY | |
| RT-4 AA | GCTGGGCAACACCGACCGGCACAGCATCAAG | | PTIYHERKKLVDSTDKADLRLIYLALAHMI | |
| linker- | AAGAACCTGATCGGAGCCCTGCTGTTCGACA | | KFRGHFLIEGDLNPDNSDVDKLFIQLVQTY | |
| bpNLS- | GCGGCGAAACAGCCGAGGCCACCCGGCTGAA | | NQLFEENPINASGVDAKAILSARLSKSRRL | |
| P2A- | GAGAACCGCCAGAAGAAGATACACCAGACGG | | ENLIAQLPGEKKNGLEGNLIALSLGLIPNE | |
| eGFP | AAGAACCGGATCTGCTATCTGCAAGAGATCT | | KSNFDLAEDAKLQLSKDTYDDDLDNLLAQI | |
| | TCAGCAACGAGATGGCCAAGGTGGACGACAG | | GDQYADLFLAAKNLSDAILLSDILRVNTEI | |
| | CTTCTTCCACAGACTGGAAGAGTCCTTCCTG | | TKAPLSASMIKRYDEHHQDLTLLKALVRQQ | |
| | GTGGAAGAGGATAAGAAGCACGAGCGGCACC | | LPEKYKEIFFDQSKNGYAGYIDGGASQEEF | |
| | CCATCTTCGGCAACATCGTGGACGAGGTGGC | | YKFIKPILEKMDGTEELLVKLNREDLLRKQ | |
| | CTACCACGAGAAGTACCCCACCATCTACCAC | | RTFDNGSIPHQIHLGELHAILRRQEDFYPF | |
| | CTGAGAAAGAAACTGGTGGACAGCACCGACA | | LKDNREKIEKILIFRIPYYVGPLARGNSRF | |
| | AGGCCGACCTGCGGCTGATCTATCTGGCCCT | | AWMTRKSEETITPWNFEEVVDKGASAQSFI | |
| | GGCCCACATGATCAAGTTCCGGGGCCACTTC | | ERMTNFDKNLPNEKVLPKASLLYEYFTVYN | |
| | CTGATCGAGGGCGACCTGAACCCCGACAACA | | ELTKVKYVTEGMRKPAFLSGEQKKAIVDLL | |
| | GCGACGTGGACAAGCTGTTCATCCAGCTGGT | | FKTNRKVTVKQLKEDYFKKIECFDSVEISG | |
| | GCAGACCTACAACCAGCTGTTCGAGGAAAAC | | VEDRFNASLGTYHDLLKIIKDKDFLDNEEN | |
| | CCCATCAACGCCAGCGGCGTGGACGCCAAGG | | EDILEDIVLTLTLFEDREMIEERLKTYAHL | |
| | CCATCCTGTCTGCCAGACTGAGCAAGAGCAG | | FDDKVMKQLKRRRYTGWGRLSRKLINGIRD | |
| | ACGGCTGGAAAATCTGATCGCCCAGCTGCCC | | KQSGKTILDFLKSDGFANRNFMQLIHDDSL | |
| | GGCGAGAAGAAGAATGGCCTGTTCGGAAACC | | TPKEDIQKAQVSGQGDSLHEHIANLAGSPA | |
| | TGATTGCCCTGAGCCTGGGCCTGACCCCCAA | | IKKGILQTVKVVDELVKVMGRHKPENIVIE | |
| | CTTCAAGAGCAACTTCGACCTGGCCGAGGAT | | MARENQTTQKGQKNSRERMKRIEEGIKELG | |
| | GCCAAACTGCAGCTGAGCAAGGACACCTACG | | SQILKEHPVENTQLQNEKLYLYYLQNGRDM | |
| | ACGACGACCTGGACAACCTGCTGGCCCAGAT | | YVDQELDINRLSDYDVDAIVPQSFLKDDSI | |
| | CGGCGACCAGTACGCCGACCTGTTTCTGGCC | | DNKVLTRSDKNRGKSDNVPSEEVVKKMKNY | |
| | GCCAAGAACCTGTCCGACGCCATCCTGCTGA | | WRQLLNAKLITQRKFDNLTKAERGGLSELD | |
| | GCGACATCCTGAGAGTGAACACCGAGATCAC | | KAGFIKRQLVETRQITKHVAQILDSRMNTK | |
| | CAAGGCCCCCCTGAGCGCCTCTATGATCAAG | | YDENDKLIREVKVITLKSKLVSDFRKDFQF | |
| | AGATACGACGAGCACCACCAGGACCTGACCC | | YKVREINNYHHAHDAYLNAVVGTALIKKYP | |
| | TGCTGAAAGCTCTCGTGCGGCAGCAGCTGCC | | KLESEFVYGDYKVYDVRKMIAKSEQEIGKA | |
| | TGAGAAGTACAAAGAGATTTTCTTCGACCAG | | TAKYFFYSNIMNFFKTEITLANGEIRKRPL | |
| | AGCAAGAACGGCTACGCCGGCTACATTGACG | | IETNGETGEIVWDKGRDFATVRKVLSMPQV | |
| | GCGGAGCCAGCCAGGAAGAGTTCTACAAGTT | | NIVKKTEVQTGGFSKESILPKRNSDKLIAR | |
| | CATCAAGCCCATCCTGGAAAAGATGGACGGC | | KKDWDPKKYGGFDSPTVAYSVLVVAKVEKG | |
| | ACCGAGGAACTGCTCGTGAAGCTGAACAGAG | | KSKKLKSVKELLGITIMERSSFEKNPIDFL | |
| | AGGACCTGCTGCGGAAGCAGCGGACCTTCGA | | EAKGYKEVKKDLIIKLPKYSLFELENGRKR | |
| | CAACGGCAGCATCCCCCACCAGATCCACCTG | | MLASAGELQKGNELALPSKYVNFLYLASHY | |
| | GGAGAGCTGCACGCCATTCTGCGGCGGCAGG | | EKLKGSPEDNEQKQLFVEQHKHYLDEIIEQ | |
| | AAGATTTTTACCCATTCCTGAAGGACAACCG | | ISEFSKRVILADANLDKVLSAYNKHRDKPI | |
| | GGAAAAGATCGAGAAGATCCTGACCTTCCGC | | REQAENIIHLFTLINLGAPAAFKYFDTTID | |
| | ATCCCCTACTACGTGGGCCCTCTGGCCAGGG | | RKRYTSTKEVLDATLIHQSITGLYETRIDL | |
| | GAAACAGCAGATTCGCCTGGATGACCAGAAA | | SQLGGDSGGSSGGSSGSETPGTSESATPES | |
| | GAGCGAGGAAACCATCACCCCCTGGAACTTC | | SGGSSGGSSDTSNLMEQILSSDNLNRAYLQ | |
| | GAGGAAGTGGTGGACAAGGGCGCTTCCGCCC | | VVRNKGAEGVDGMKYTELKEHLAKNGETIK | |
| | AGAGCTTCATCGAGCGGATGACCAACTTCGA | | GQLRTRKYKPQPARRVEIPKPDGGVRNLGV | |
| | TAAGAACCTGCCCAACGAGAAGGTGCTGCCC | | PTVTDRFIQQAIAQVLTPIYEEQFHDHSYG | |
| | AAGCACAGCCTGCTGTACGAGTACTTCACCG | | FRPNRCAQQAILTALNIMNDGNDWIVDIDL | |
| | TGTATAACGAGCTGACCAAAGTGAAATACGT | | EKFFDTVNHDKLMTLIGRTIKDGDVISIVR | |
| | GACCGAGGGAATGAGAAAGCCCGCCTTCCTG | | KYLVSGIMIDDEYEDSIVGTPQGGNLSPLL | |
| | AGCGGCGAGCAGAAAAAGGCCATCGTGGACC | | ANIMLNELDKEMEKRGLNFVRYADDCIIMV | |
| | TGCTGTTCAAGACCAACCGGAAAGTGACCGT | | GSEMSANRVMRNISRFIEEKLGLKVNMTKS | |
| | GAAGCAGCTGAAAGAGGACTACTTCAAGAAA | | KVDRPSGLKYLGFGFYFDPRAHQFKAKPHA | |
| | ATCGAGTGCTTCGACTCCGTGGAAATCTCCG | | KSVAKFKKRMKELTCRSWGVSNSYKVEKLN | |
| | GCGTGGAAGATCGGTTCAACGCCTCCCTGGG | | QLIRGWINYFKIGSMKTLCKELDSRIRYRL | |
| | CACATACCACGATCTGCTGAAAATTATCAAG | | RMCIWKQWKTPQNQEKNLVKLGIDRNTARR | |
| | GACAAGGACTTCCTGGACAATGAGGAAAACG | | VAYTGKRIAYVCNKGAVNVAISNKRLASFG | |
| | AGGACATTCTGGAAGATATCGTGCTGACCCT | | LISMLDYYIEKCVTCSGGSKRTADGSEFEP | |
| | GACACTGTTTGAGGACAGAGAGATGATCGAG | | KKKRKVGSGATNFSLLKQAGDVEENPGPMV | |
| | GAACGGCTGAAAACCTATGCCCACCTGTTCG | | SKGEELFTGVVPILVELDGDVNGHKFSVSG | |
| | ACGACAAAGTGATGAAGCAGCTGAAGCGGCG | | EGEGDATYGKLTLKFICTTGKLPVPWPTLV | |
| | GAGATACACCGGCTGGGGCAGGCTGAGCCGG | | TTLTYGVQCFSRYPDHMKQHDFFKSAMPEG | |
| | AAGCTGATCAACGGCATCCGGGACAAGCAGT | | YVQERTIFFKDDGNYKTRAEVKFEGDTLVN | |
| | CCGGCAAGACAATCCTGGATTTCCTGAAGTC | | RIELKGIDFKEDGNILGHKLEYNYNSHNVY | |
| | CGACGGCTTCGCCAACAGAAACTTCATGCAG | | IMADKQKNGIKVNFKIRHNIEDGSVQLADH | |
| | CTGATCCACGACGACAGCCTGACCTTTAAAG | | YQQNTPIGDGPVLLPDNHYLSTQSALSKDP | |
| | AGGACATCCAGAAAGCCCAGGTGTCCGGCCA | | NEKRDHMVLLEFVTAAGITLGMDELYK* | |
| | GGGCGATAGCCTGCACGAGCACATTGCCAAT | | | |
| | CTGGCCGGCAGCCCCGCCATTAAGAAGGGCA | | | |
| | TCCTGCAGACAGTGAAGGTGGTGGACGAGCT | | | |
| | CGTGAAAGTGATGGGCCGGCACAAGCCCGAG | | | |
| | AACATCGTGATCGAAATGGCCAGAGAGAACC | | | |
| | AGACCACCCAGAAGGGACAGAAGAACAGCCG | | | |
| | CGAGAGAATGAAGCGGATCGAAGAGGGCATC | | | |
| | AAAGAGCTGGGCAGCCAGATCCTGAAAGAAC | | | |
| | ACCCCGTGGAAAACACCCAGCTGCAGAACGA | | | |
| | GAAGCTGTACCTGTACTACCTGCAGAATGGG | | | |
| | CGGGATATGTACGTGGACCAGGAACTGGACA | | | |
| | TCAACCGGCTGTCCGACTACGATGTGGACGC | | | |
| | TATCGTGCCTCAGAGCTTTCTGAAGGACGAC | | | |
| | TCCATCGACAACAAGGTGCTGACCAGAAGCG | | | |
| | ACAAGAACCGGGGCAAGAGCGACAACGTGCC | | | |
| | CTCCGAAGAGGTCGTGAAGAAGATGAAGAAC | | | |
| | TACTGGCGGCAGCTGCTGAACGCCAAGCTGA | | | |
| | TTACCCAGAGAAAGTTCGACAATCTGACCAA | | | |
| | GGCCGAGAGAGGCGGCCTGAGCGAACTGGAT | | | |
| | AAGGCCGGCTTCATCAAGAGACAGCTGGTGG | | | |
| | AAACCCGGCAGATCACAAAGCACGTGGCACA | | | |
| | GATCCTGGACTCCCGGATGAACACTAAGTAC | | | |
| | GACGAGAATGACAAGCTGATCCGGGAAGTGA | | | |
| | AAGTGATCACCCTGAAGTCCAAGCTGGTGTC | | | |
| | CGATTTCCGGAAGGATTTCCAGTTTTACAAA | | | |
| | GTGCGCGAGATCAACAACTACCACCACGCCC | | | |
| | ACGACGCCTACCTGAACGCCGTCGTGGGAAC | | | |
| | CGCCCTGATCAAAAAGTACCCTAAGCTGGAA | | | |
| | AGCGAGTTCGTGTACGGCGACTACAAGGTGT | | | |
| | ACGACGTGCGGAAGATGATCGCCAAGAGCGA | | | |
| | GCAGGAAATCGGCAAGGCTACCGCCAAGTAC | | | |
| | TTCTTCTACAGCAACATCATGAACTTTTTCA | | | |
| | AGACCGAGATTACCCTGGCCAACGGCGAGAT | | | |
| | CCGGAAGCGGCCTCTGATCGAGACAAACGGC | | | |
| | GAAACCGGGGAGATCGTGTGGGATAAGGGCC | | | |
| | GGGATTTTGCCACCGTGCGGAAAGTGCTGAG | | | |
| | CATGCCCCAAGTGAATATCGTGAAAAAGACC | | | |
| | GAGGTGCAGACAGGCGGCTTCAGCAAAGAGT | | | |
| | CTATCCTGCCCAAGAGGAACAGCGATAAGCT | | | |
| | GATCGCCAGAAAGAAGGACTGGGACCCTAAG | | | |
| | AAGTACGGCGGCTTCGACAGCCCCACCGTGG | | | |
| | CCTATTCTGTGCTGGTGGTGGCCAAAGTGGA | | | |
| | AAAGGGCAAGTCCAAGAAACTGAAGAGTGTG | | | |
| | AAAGAGCTGCTGGGGATCACCATCATGGAAA | | | |
| | GAAGCAGCTTCGAGAAGAATCCCATCGACTT | | | |
| | TCTGGAAGCCAAGGGCTACAAAGAAGTGAAA | | | |
| | AAGGACCTGATCATCAAGCTGCCTAAGTACT | | | |
| | CCCTGTTCGAGCTGGAAAACGGCCGGAAGAG | | | |
| | AATGCTGGCCTCTGCCGGCGAACTGCAGAAG | | | |
| | GGAAACGAACTGGCCCTGCCCTCCAAATATG | | | |
| | TGAACTTCCTGTACCTGGCCAGCCACTATGA | | | |
| | GAAGCTGAAGGGCTCCCCCGAGGATAATGAG | | | |
| | CAGAAACAGCTGTTTGTGGAACAGCACAAGC | | | |
| | ACTACCTGGACGAGATCATCGAGCAGATCAG | | | |
| | CGAGTTCTCCAAGAGAGTGATCCTGGCCGAC | | | |
| | GCTAATCTGGACAAAGTGCTGTCCGCCTACA | | | |
| | ACAAGCACCGGGATAAGCCCATCAGAGAGCA | | | |
| | GGCCGAGAATATCATCCACCTGTTTACCCTG | | | |
| | ACCAATCTGGGAGCCCCTGCCGCCTTCAAGT | | | |
| | ACTTTGACACCACCATCGACCGGAAGAGGTA | | | |
| | CACCAGCACCAAAGAGGTGCTGGACGCCACC | | | |
| | CTGATCCACCAGAGCATCACCGGCCTGTACG | | | |
| | AGACACGGATCGACCTGTCTCAGCTGGGAGG | | | |
| | TGACTCTGGAGGATCTAGCGGAGGATCCTCT | | | |
| | GGCAGCGAGACACCAGGAACAAGCGAGTCAG | | | |
| | CAACACCAGAGAGCAGTGGCGGCAGCAGCGG | | | |
| | CGGCAGCAGCGACACCAGCAATCTGATGGAA | | | |
| | CAGATCCTGAGCAGCGACAACCTGAACCGGG | | | |
| | CCTACCTGCAGGTGGTGAGAAATAAAGGCGC | | | |
| | TGAAGGCGTTGATGGCATGAAGTACACCGAG | | | |
| | CTGAAGGAGCATCTGGCCAAGAACGGCGAGA | | | |
| | CAATCAAGGGCCAGCTGAGAACCAGAAAGTA | | | |
| | TAAGCCTCAGCCAGCTAGACGGGTGGAAATC | | | |
| | CCCAAGCCCGATGGCGGAGTGCGGAACCTGG | | | |
| | GAGTGCCAACAGTCACAGACCGGTTCATCCA | | | |
| | GCAGGCTATCGCCCAAGTGCTGACCCCTATC | | | |
| | TACGAGGAACAGTTTCACGACCACTCTTACG | | | |
| | GCTTCCGGCCCAACAGATGCGCCCAGCAAGC | | | |
| | CATCCTGACAGCCCTGAACATCATGAACGAT | | | |
| | GGTAATGACTGGATCGTGGACATCGACCTGG | | | |
| | AAAAGTTTTTCGATACCGTGAATCACGATAA | | | |
| | GCTGATGACGCTGATTGGCAGAACCATCAAG | | | |
| | GACGGCGACGTGATCTCTATTGTGCGCAAGT | | | |
| | ACCTCGTGTCCGGCATCATGATCGATGACGA | | | |
| | GTACGAAGATAGCATCGTGGGAACACCTCAG | | | |
| | GGCGGCAACCTGTCTCCTCTGCTGGCCAACA | | | |
| | TCATGCTGAACGAGCTGGATAAGGAGATGGA | | | |
| | AAAAAGGGGCCTGAACTTCGTGCGGTACGCC | | | |
| | GACGACTGCATCATCATGGTCGGCTCCGAGA | | | |
| | TGAGCGCCAACAGAGTCATGCGGAACATCAG | | | |
| | CAGATTCATCGAAGAGAAGCTGGGCCTGAAA | | | |
| | GTGAACATGACCAAGTCCAAGGTGGACAGAC | | | |
| | CTAGCGGACTGAAGTACTTGGGCTTTGGCTT | | | |
| | CTACTTCGACCCCAGAGCCCACCAGTTCAAG | | | |
| | GCCAAGCCTCACGCCAAGAGCGTGGCTAAGT | | | |
| | TCAAAAAGAGAATGAAAGAGCTGACCTGTAG | | | |
| | AAGCTGGGGCGTGTCTAACAGCTACAAGGTG | | | |
| | GAAAAACTGAATCAACTGATCAGAGGCTGGA | | | |
| | TCAACTACTTCAAGATCGGCAGCATGAAGAC | | | |
| | CCTGTGTAAAGAGCTGGACAGCAGAATCAGG | | | |
| | TACAGACTGCGGATGTGCATCTGGAAGCAGT | | | |
| | GGAAAACCCCTCAGAACCAGGAGAAAAACCT | | | |
| | GGTCAAGCTTGGAATTGACAGAAATACCGCC | | | |
| | AGAAGAGTGGCCTATACAGGCAAGCGAATCG | | | |
| | CCTACGTGTGCAACAAGGGCGCCGTGAACGT | | | |
| | GGCTATCAGCAACAAGCGGCTGGCCAGCTTC | | | |
| | GGCCTGATCTCTATGCTGGACTACTACATCG | | | |
| | AGAAGTGCGTGACCTGCTCTGGCGGCTCAAA | | | |
| | AAGAACCGCCGACGGCAGCGAATTCGAGCCC | | | |
| | AAGAAGAAGAGGAAAGTCGGAAGCGGAGCTA | | | |
| | CTAACTTCAGCCTGCTGAAGCAGGCTGGAGA | | | |
| | CGTGGAGGAGAACCCTGGACCTATGGTGAGC | | | |
| | AAGGGCGAGGAGCTGTTCACCGGGGTGGTGC | | | |
| | CCATCCTGGTCGAGCTGGACGGCGACGTAAA | | | |
| | CGGCCACAAGTTCAGCGTGTCCGGCGAGGGC | | | |
| | GAGGGCGATGCCACCTACGGCAAGCTGACCC | | | |
| | TGAAGTTCATCTGCACCACCGGCAAGCTGCC | | | |
| | CGTGCCCTGGCCCACCCTCGTGACCACCCTG | | | |
| | ACCTATGGAGTGCAGTGCTTCAGCCGCTACC | | | |
| | CCGACCACATGAAGCAGCACGACTTCTTCAA | | | |
| | GTCCGCCATGCCCGAAGGCTACGTCCAGGAG | | | |
| | CGCACCATCTTCTTCAAGGACGACGGCAACT | | | |
| | ACAAGACCCGCGCCGAGGTGAAGTTCGAGGG | | | |
| | CGACACCCTGGTGAACCGCATCGAGCTGAAG | | | |
| | GGCATCGACTTCAAGGAGGACGGCAACATCC | | | |
| | TGGGGCACAAGCTGGAGTACAACTACAACAG | | | |
| | CCACAACGTCTATATCATGGCCGACAAGCAG | | | |
| | AAGAACGGCATCAAGGTGAACTTCAAGATCC | | | |
| | GCCACAACATCGAGGACGGCAGCGTGCAGCT | | | |
| | CGCCGACCACTACCAGCAGAACACCCCCATC | | | |
| | GGCGACGGCCCCGTGCTGCTGCCCGACAACC | | | |
| | ACTACCTGAGCACCCAGTCCGCCCTGAGCAA | | | |
| | AGACCCCAACGAGAAGCGCGATCACATGGTC | | | |
| | CTGCTGGAGTTCGTGACCGCCGCCGGGATCA | | | |
| | CTCTCGGCATGGACGAGCTGTACAAGTAA | | | |
| bpNLS- | ATGAAACGGACAGCCGACGGAAGCGAGTTCG | 118 | MKRTADGSEFESPKKKRKVDKKYSIGLDIG | 119 |
| nCas9 | AGTCACCAAAGAAGAAGCGGAAAGTCGACAA | | TNSVGWAVITDEYKVPSKKFKVLGNTDRHS | |
| (H840A) | GAAGTACAGCATCGGCCTGGACATCGGCACC | | IKKNLIGALLFDSGETAEATRLKRTARRRY | |
| -XTEN- | AACTCTGTGGGCTGGGCCGTGATCACCGACG | | TRRKNRICYLQEIFSNEMAKVDDSFFHRLE | |
| Marathon | AGTACAAGGTGCCCAGCAAGAAATTCAAGGT | | ESFLVEEDKKHERHPIFGNIVDEVAYHEKY | |
| RT | GCTGGGCAACACCGACCGGCACAGCATCAAG | | PTIYHLRKKLVDSTDKADLRLIYLALAHMI | |
| (D14R- | AAGAACCTGATCGGAGCCCTGCTGTTCGACA | | KERGHFLIEGDLNPDNSDVDKLFIQLVQTY | |
| D74R- | GCGGCGAAACAGCCGAGGCCACCCGGCTGAA | | NQLFEENPINASGVDAKAILSARLSKSRRL | |
| N116K- | GAGAACCGCCAGAAGAAGATACACCAGACGG | | ENLIAQLPGEKKNGLFGNLIALSLGLIPNF | |
| N197R) | AAGAACCGGATCTGCTATCTGCAAGAGATCT | | KSNFDLAEDAKLQLSKDTYDDDLQNLLAQI | |
| -4 AA | TCAGCAACGAGATGGCCAAGGTGGACGACAG | | GDQYADLFLAAKNLSDAILLSDILRVNTEI | |
| linker- | CTTCTTCCACAGACTGGAAGAGTCCTTCCTG | | TKAPLSASMIKRYDEHHQDLTLLKALVRQQ | |
| bpNLS- | GTGGAAGAGGATAAGAAGCACGAGCGGCACC | | LPEKYKEIFFDQSKNGYAGYIDGGASQEEF | |
| P2A- | CCATCTTCGGCAACATCGTGGACGAGGTGGC | | YKFIKPILEKMDGTEELLVKLNREDLLRKQ | |
| eGPP | CTACCACGAGAAGTACCCCACCATCTACCAC | | RTFDNGSIPHQIHLGELHAILRRQEDFYPF | |
| | CTGAGAAAGAAACTGGTGGACAGCACCGACA | | LKDNREKIEKILTFRIPYYVGPLARGNSRF | |
| | AGGCCGACCTGCGGCTGATCTATCTGGCCCT | | AWMTRKSEETITPWNFEEVVDKGASAQSFI | |
| | GGCCCACATGATCAAGTTCCGGGGCCACTTC | | ERMTNFDKNLPNEKVLPKHSLLYEYETVYN | |
| | CTGATCGAGGGCGACCTGAACCCCGACAACA | | ELTKVKYVTEGMRKPAFLSGEQKKAIVDLL | |
| | GCGACGTGGACAAGCTGTTCATCCAGCTGGT | | FKTNRKVTVKQLKEDYFKKIECFDSVEISG | |
| | GCAGACCTACAACCAGCTGTTCGAGGAAAAC | | VEDRFNASLGTYHDLLKIIKDKDFLDNEEN | |
| | CCCATCAACGCCAGCGGCGTGGACGCCAAGG | | EDILEDIVLTLTLFEDREMIEERLKTYAHL | |
| | CCATCCTGTCTGCCAGACTGAGCAAGAGCAG | | FDDKVMKQLKRRRYTGWGRLSRKLINGIRD | |
| | ACGGCTGGAAAATCTGATCGCCCAGCTGCCC | | KQSGKTILDFLKSDGFANRNFMQLIHDDSL | |
| | GGCGAGAAGAAGAATGGCCTGTTCGGAAACC | | TFKEDIQKAQVSGQGDSLHEHIANLAGSPA | |
| | TGATTGCCCTGAGCCTGGGCCTGACCCCCAA | | IKKGILQTVKVVDELVKVMGRHKPENIVIE | |
| | CTTCAAGAGCAACTTCGACCTGGCCGAGGAT | | MARENQTTQKGQKNSRERMKRIEEGIKELG | |
| | GCCAAACTGCAGCTGAGCAAGGACACCTACG | | SQILKEHPVENTQLQNEKLYLYYLQNGRDM | |
| | ACGACGACCTGGACAACCTGCTGGCCCAGAT | | YVDQELDINRLSDYDVDAIVPQSFLKDDSI | |
| | CGGCGACCAGTACGCCGACCTGTTTCTGGCC | | DNKVLIRSDKNRGKSDNVPSEEVVKKMKNY | |
| | GCCAAGAACCTGTCCGACGCCATCCTGCTGA | | WRQLLNAKLITQRKEDNITKAERGGLSELD | |
| | GCGACATCCTGAGAGTGAACACCGAGATCAC | | KAGFIKRQLVETRQITKHVAQILDSRMNTK | |
| | CAAGGCCCCCCTGAGCGCCTCTATGATCAAG | | YDENDKLIREVKVITLKSKLVSDFRKDFQF | |
| | AGATACGACGAGCACCACCAGGACCTGACCC | | YKVREINNYHHAHDAYLNAVVGTALIKKYP | |
| | TGCTGAAAGCTCTCGTGCGGCAGCAGCTGCC | | KLESEFVYGDYKVYDVRKMIAKSEQEIGKA | |
| | TGAGAAGTACAAAGAGATTTTCTTCGACCAG | | TAKYFFYSNIMNFFKTEITLANGEIRKRPL | |
| | AGCAAGAACGGCTACGCCGGCTACATTGACG | | IETNGETGEIVWDKGRDFATVRKVLSMPQV | |
| | GCGGAGCCAGCCAGGAAGAGTTCTACAAGTT | | NIVKKTEVQTGGFSKESILPKRNSDKLIAR | |
| | CATCAAGCCCATCCTGGAAAAGATGGACGGC | | KKDWDPKKYGGFDSPTVAYSVLVVAKVEKG | |
| | ACCGAGGAACTGCTCGTGAAGCTGAACAGAG | | KSKKLKSVKELLGITIMERSSFEKNPIDEL | |
| | AGGACCTGCTGCGGAAGCAGCGGACCTTCGA | | EAKGYKEVKKDLIIKLPKYSLFELENGRKR | |
| | CAACGGCAGCATCCCCCACCAGATCCACCTG | | MLASAGELQKGNELALPSKYVNFLYLASHY | |
| | GGAGAGCTGCACGCCATTCTGCGGCGGCAGG | | EKLKGSPEDNEQKQLFVEQHKHYLDEIIEQ | |
| | AAGATTTTTACCCATTCCTGAAGGACAACCG | | ISEFSKRVILADANLDKVLSAYNKHRDKPI | |
| | GGAAAAGATCGAGAAGATCCTGACCTTCCGC | | REQAENIIHLFTLINLGAPAAFKYFDTTID | |
| | ATCCCCTACTACGTGGGCCCTCTGGCCAGGG | | RKRYTSTKEVLDATLIHQSITGLYETRIDL | |
| | GAAACAGCAGATTCGCCTGGATGACCAGAAA | | SQLGGDSGGSSGGSSGSETPGTSESATPES | |
| | GAGCGAGGAAACCATCACCCCCTGGAACTTC | | SGGSSGGSSDTSNLMEQILSSRNLNRAYLQ | |
| | GAGGAAGTGGTGGACAAGGGCGCTTCCGCCC | | VVRRKGAEGVDGMKYTELKEHLAKNGETIK | |
| | AGAGCTTCATCGAGCGGATGACCAACTTCGA | | GQLRTRKYKPQPARRVEIPKPRGGVRNLGV | |
| | TAAGAACCTGCCCAACGAGAAGGTGCTGCCC | | PTVTDRFIQQAIAQVLTPIYERQFHDHSYG | |
| | AAGCACAGCCTGCTGTACGAGTACTTCACCG | | FRPKRCAQQAILTALNIMNDGNDWIVDIDL | |
| | TGTATAACGAGCTGACCAAAGTGAAATACGT | | EKFFDTVNHDKLMTLIGRTIKDGDVISIVR | |
| | GACCGAGGGAATGAGAAAGCCCGCCTTCCTG | | KYLVSGIMIDDEYEDSIVGTPQGGRLSPLL | |
| | AGCGGCGAGCAGAAAAAGGCCATCGTGGACC | | ANIMLNELDKEMEKRGLNFVRYADDCIIMV | |
| | TGCTGTTCAAGACCAACCGGAAAGTGACCGT | | GSEMSANRVMRNISRFIEEKLGLKVNMTKS | |
| | GAAGCAGCTGAAAGAGGACTACTTCAAGAAA | | KVDRPSGLKYLGFGFYFDPRAHQFKAKPHA | |
| | ATCGAGTGCTTCGACTCCGTGGAAATCTCCG | | KSVAKFKKRMKELTCRSWGVSNSYKVEKLN | |
| | GCGTGGAAGATCGGTTCAACGCCTCCCTGGG | | QLIRGWINYFKIGSMKTLCKELDSRIRYRL | |
| | CACATACCACGATCTGCTGAAAATTATCAAG | | RMCIWKQWKTPQNQEKNLVKLGIDRNTARR | |
| | GACAAGGACTTCCTGGACAATGAGGAAAACG | | VAYTGKRIAYVCNKGAVNVAISNKRLASEG | |
| | AGGACATTCTGGAAGATATCGTGCTGACCCT | | LISMLDYYIEKCVTCSGGSKRTADGSEFEP | |
| | GACACTGTTTGAGGACAGAGAGATGATCGAG | | KKKRKVGSGATNFSLLKQAGDVEENPGPMV | |
| | GAACGGCTGAAAACCTATGCCCACCTGTTCG | | SKGEELFTGVVPILVELDGDVNGHKFSVSG | |
| | ACGACAAAGTGATGAAGCAGCTGAAGCGGCG | | EGEGDATYGKLTLKFICTTGKLPVPWPTLV | |
| | GAGATACACCGGCTGGGGCAGGCTGAGCCGG | | TTLTYGVQCFSRYPDHMKQHDFFKSAMPEG | |
| | AAGCTGATCAACGGCATCCGGGACAAGCAGT | | YVQERTIFFKDDGNYKTRAEVKFEGDTLVN | |
| | CCGGCAAGACAATCCTGGATTTCCTGAAGTC | | RIELKGIDFKEDGNILGHKLEYNYNSHNVY | |
| | CGACGGCTTCGCCAACAGAAACTTCATGCAG | | IMADKQKNGIKVNFKIRHNIEDGSVQLADH | |
| | CTGATCCACGACGACAGCCTGACCTTTAAAG | | YQQNTPIGDGPVLLPDNHYLSTQSALSKDP | |
| | AGGACATCCAGAAAGCCCAGGTGTCCGGCCA | | NEKRDHMVLLEFVTAAGITLGMDELYK* | |
| | GGGCGATAGCCTGCACGAGCACATTGCCAAT | | | |
| | CTGGCCGGCAGCCCCGCCATTAAGAAGGGCA | | | |
| | TCCTGCAGACAGTGAAGGTGGTGGACGAGCT | | | |
| | CGTGAAAGTGATGGGCCGGCACAAGCCCGAG | | | |
| | AACATCGTGATCGAAATGGCCAGAGAGAACC | | | |
| | AGACCACCCAGAAGGGACAGAAGAACAGCCG | | | |
| | CGAGAGAATGAAGCGGATCGAAGAGGGCATC | | | |
| | AAAGAGCTGGGCAGCCAGATCCTGAAAGAAC | | | |
| | ACCCCGTGGAAAACACCCAGCTGCAGAACGA | | | |
| | GAAGCTGTACCTGTACTACCTGCAGAATGGG | | | |
| | CGGGATATGTACGTGGACCAGGAACTGGACA | | | |
| | TCAACCGGCTGTCCGACTACGATGTGGACGC | | | |
| | TATCGTGCCTCAGAGCTTTCTGAAGGACGAC | | | |
| | TCCATCGACAACAAGGTGCTGACCAGAAGCG | | | |
| | ACAAGAACCGGGGCAAGAGCGACAACGTGCC | | | |
| | CTCCGAAGAGGTCGTGAAGAAGATGAAGAAC | | | |
| | TACTGGCGGCAGCTGCTGAACGCCAAGCTGA | | | |
| | TTACCCAGAGAAAGTTCGACAATCTGACCAA | | | |
| | GGCCGAGAGAGGCGGCCTGAGCGAACTGGAT | | | |
| | AAGGCCGGCTTCATCAAGAGACAGCTGGTGG | | | |
| | AAACCCGGCAGATCACAAAGCACGTGGCACA | | | |
| | GATCCTGGACTCCCGGATGAACACTAAGTAC | | | |
| | GACGAGAATGACAAGCTGATCCGGGAAGTGA | | | |
| | AAGTGATCACCCTGAAGTCCAAGCTGGTGTC | | | |
| | CGATTTCCGGAAGGATTTCCAGTTTTACAAA | | | |
| | GTGCGCGAGATCAACAACTACCACCACGCCC | | | |
| | ACGACGCCTACCTGAACGCCGTCGTGGGAAC | | | |
| | CCCCCTGATCAAAAAGTACCCTAAGCTGGAA | | | |
| | AGCGAGTTCGTGTACGGCGACTACAAGGTGT | | | |
| | ACGACGTGCGGAAGATGATCGCCAAGAGCGA | | | |
| | GCAGGAAATCGGCAAGGCTACCGCCAAGTAC | | | |
| | TTCTTCTACAGCAACATCATGAACTTTTTCA | | | |
| | AGACCGAGATTACCCTGGCCAACGGCGAGAT | | | |
| | CCGGAAGCGGCCTCTGATCGAGACAAACGGC | | | |
| | GAAACCGGGGAGATCGTGTGGGATAAGGGCC | | | |
| | GGGATTTTGCCACCGTGCGGAAAGTGCTGAG | | | |
| | CATGCCCCAAGTGAATATCGTGAAAAAGACC | | | |
| | GAGGTGCAGACAGGCGGCTTCAGCAAAGAGT | | | |
| | CTATCCTGCCCAAGAGGAACAGCGATAAGCT | | | |
| | GATCGCCAGAAAGAAGGACTGGGACCCTAAG | | | |
| | AAGTACGGCGGCTTCGACAGCCCCACCGTGG | | | |
| | CCTATTCTGTGCTGGTGGTGGCCAAAGTGGA | | | |
| | AAAGGGCAAGTCCAAGAAACTGAAGAGTGTG | | | |
| | AAAGAGCTGCTGGGGATCACCATCATGGAAA | | | |
| | GAAGCAGCTTCGAGAAGAATCCCATCGACTT | | | |
| | TCTGGAAGCCAAGGGCTACAAAGAAGTGAAA | | | |
| | AAGGACCTGATCATCAAGCTGCCTAAGTACT | | | |
| | CCCTGTTCGAGCTGGAAAACGGCCGGAAGAG | | | |
| | AATGCTGGCCTCTGCCGGCGAACTGCAGAAG | | | |
| | GGAAACGAACTGGCCCTGCCCTCCAAATATG | | | |
| | TGAACTTCCTGTACCTGGCCAGCCACTATGA | | | |
| | GAAGCTGAAGGGCTCCCCCGAGGATAATGAG | | | |
| | CAGAAACAGCTGTTTGTGGAACAGCACAAGC | | | |
| | ACTACCTGGACGAGATCATCGAGCAGATCAG | | | |
| | CGAGTTCTCCAAGAGAGTGATCCTGGCCGAC | | | |
| | GCTAATCTGGACAAAGTGCTGTCCGCCTACA | | | |
| | ACAAGCACCGGGATAAGCCCATCAGAGAGCA | | | |
| | GGCCGAGAATATCATCCACCTGTTTACCCTG | | | |
| | ACCAATCTGGGAGCCCCTGCCGCCTTCAAGT | | | |
| | ACTTTGACACCACCATCGACCGGAAGAGGTA | | | |
| | CACCAGCACCAAAGAGGTGCTGGACGCCACC | | | |
| | CTGATCCACCAGAGCATCACCGGCCTGTACG | | | |
| | AGACACGGATCGACCTGTCTCAGCTGGGAGG | | | |
| | TGACTCTGGAGGATCTAGCGGAGGATCCTCT | | | |
| | GGCAGCGAGACACCAGGAACAAGCGAGTCAG | | | |
| | CAACACCAGAGAGCAGTGGCGGCAGCAGCGG | | | |
| | CGGCAGCAGCGACACCAGCAATCTGATGGAA | | | |
| | CAGATCCTGAGCAGCCGGAACCTGAACCGGG | | | |
| | CCTACCTGCAGGTGGTGAGACGGAAAGGCGC | | | |
| | TGAAGGCGTTGATGGCATGAAGTACACCGAG | | | |
| | CTGAAGGAGCATCTGGCCAAGAACGGCGAGA | | | |
| | CAATCAAGGGCCAGCTGAGAACCAGAAAGTA | | | |
| | TAAGCCTCAGCCAGCTAGACGGGTGGAAATC | | | |
| | CCCAAGCCCCGGGGCGGAGTGCGGAACCTGG | | | |
| | GAGTGCCAACAGTCACAGACCGGTTCATCCA | | | |
| | GCAGGCTATCGCCCAAGTGCTGACCCCTATC | | | |
| | TACGAGGAACAGTTTCACGACCACTCTTACG | | | |
| | GCTTCCGGCCCAAGAGATGCGCCCAGCAAGC | | | |
| | CATCCTGACAGCCCTGAACATCATGAACGAT | | | |
| | GGTAATGACTGGATCGTGGACATCGACCTGG | | | |
| | AAAAGTTTTTCGATACCGTGAATCACGATAA | | | |
| | GCTGATGACGCTGATTGGCAGAACCATCAAG | | | |
| | GACGGCGACGTGATCTCTATTGTGCGCAAGT | | | |
| | ACCTCGTGTCCGGCATCATGATCGATGACGA | | | |
| | GTACGAAGATAGCATCGTGGGAACACCTCAG | | | |
| | GGCGGCCGGCTGTCTCCTCTGCTGGCCAACA | | | |
| | TCATGCTGAACGAGCTGGATAAGGAGATGGA | | | |
| | AAAAAGGGGCCTGAACTTCGTGCGGTACGCC | | | |
| | GACGACTGCATCATCATGGTCGGCTCCGAGA | | | |
| | TGAGCGCCAACAGAGTCATGCGGAACATCAG | | | |
| | CAGATTCATCGAAGAGAAGCTGGGCCTGAAA | | | |
| | GTGAACATGACCAAGTCCAAGGTGGACAGAC | | | |
| | CTAGCGGACTGAAGTACTTGGGCTTTGGCTT | | | |
| | CTACTTCGACCCCAGAGCCCACCAGTTCAAG | | | |
| | GCCAAGCCTCACGCCAAGAGCGTGGCTAAGT | | | |
| | TCAAAAAGAGAATGAAAGAGCTGACCTGTAG | | | |
| | AAGCTGGGGCGTGTCTAACAGCTACAAGGTG | | | |
| | GAAAAACTGAATCAACTGATCAGAGGCTGGA | | | |
| | TCAACTACTTCAAGATCGGCAGCATGAAGAC | | | |
| | CCTGTGTAAAGAGCTGGACAGCAGAATCAGG | | | |
| | TACAGACTGCGGATGTGCATCTGGAAGCAGT | | | |
| | GGAAAACCCCTCAGAACCAGGAGAAAAACCT | | | |
| | GGTCAAGCTTGGAATTGACAGAAATACCGCC | | | |
| | AGAAGAGTGGCCTATACAGGCAAGCGAATCG | | | |
| | CCTACGTGTGCAACAAGGGCGCCGTGAACGT | | | |
| | GGCTATCAGCAACAAGCGGCTGGCCAGCTTC | | | |
| | GGCCTGATCTCTATGCTGGACTACTACATCG | | | |
| | AGAAGTGCGTGACCTGCTCTGGCGGCTCAAA | | | |
| | AAGAACCGCCGACGGCAGCGAATTCGAGCCC | | | |
| | AAGAAGAAGAGGAAAGTCGGAAGCGGAGCTA | | | |
| | CTAACTTCAGCCTGCTGAAGCAGGCTGGAGA | | | |
| | CGTGGAGGAGAACCCTGGACCTATGGTGAGC | | | |
| | AAGGGCGAGGAGCTGTTCACCGGGGTGGTGC | | | |
| | CCATCCTGGTCGAGCTGGACGGCGACGTAAA | | | |
| | CGGCCACAAGTTCAGCGTGTCCGGCGAGGGC | | | |
| | GAGGGCGATGCCACCTACGGCAAGCTGACCC | | | |
| | TGAAGTTCATCTGCACCACCGGCAAGCTGCC | | | |
| | CGTGCCCTGGCCCACCCTCGTGACCACCCTG | | | |
| | ACCTATGGAGTGCAGTGCTTCAGCCGCTACC | | | |
| | CCGACCACATGAAGCAGCACGACTTCTTCAA | | | |
| | GTCCGCCATGCCCGAAGGCTACGTCCAGGAG | | | |
| | CGCACCATCTTCTTCAAGGACGACGGCAACT | | | |
| | ACAAGACCCGCGCCGAGGTGAAGTTCGAGGG | | | |
| | CGACACCCTGGTGAACCGCATCGAGCTGAAG | | | |
| | GGCATCGACTTCAAGGAGGACGGCAACATCC | | | |
| | TGGGGCACAAGCTGGAGTACAACTACAACAG | | | |
| | CCACAACGTCTATATCATGGCCGACAAGCAG | | | |
| | AAGAACGGCATCAAGGTGAACTTCAAGATCC | | | |
| | GCCACAACATCGAGGACGGCAGCGTGCAGCT | | | |
| | CGCCGACCACTACCAGCAGAACACCCCCATC | | | |
| | GGCGACGGCCCCGTGCTGCTGCCCGACAACC | | | |
| | ACTACCTGAGCACCCAGTCCGCCCTGAGCAA | | | |
| | AGACCCCAACGAGAAGCGCGATCACATGGTC | | | |
| | CTGCTGGAGTTCGTGACCGCCGCCGGGATCA | | | |
| | CTCTCGGCATGGACGAGCTGTACAAGTAA | | | |

### REFERENCES

1. Anzalone, A. V. et al. Search-and-replace genome editing without double-strand breaks or donor DNA. Nature 576, 149-157 (2019).
2. Ran, F. A. et al. In vivo genome editing using Staphylococcus aureus Cas9. Nature 520, 186-191 (2015).
3. Wang. Y., Zhou, L., Liu. N. & Yao, S. BE-PIGS: a base-editing tool with deaminases inlaid into Cas9 PI domain significantly expanded the editing scope. Signal Transduct Target Ther 4, 36 (2019).
4. Kleinstiver, B. P. et al. Broadening the targeting range of Staphylococcus aureus CRISPR-Cas9 by modifying PAM recognition. Nat Biotechnol 33, 1293-1298 (2015).
5. Gu, J., Villanueva. R. A., Snyder, C. S., Roth, M. J. & Georgiadis, M. M. Substitution of Asp 114 or Arg116 in the fingers domain of moloney murine leukemia virus reverse transcriptase affects interactions with the template-primer resulting in decreased processivity. J Mol Biol 305, 341-359 (2001).
6. Das, D. & Georgiadis, M. M. A directed approach to improving the solubility of Moloney murine leukemia virus reverse transcriptase. Protein Sci 10, 1936-1941 (2001).
7. Katano, Y. et al. Generation of thermostable Moloney murine leukemia virus reverse transcriptase variants using site saturation mutagenesis library and cell-free protein expression system. Biosci Biotechnol Biochem 81, 2339-2345 (2017).
8. Cote, M. L. & Roth, M. J. Murine leukemia virus reverse transcriptase: structural comparison with HIV-1 reverse transcriptase. Virus Res 134, 186-202 (2008).
9. Das, D. & Georgiadis, M. M. The crystal structure of the monomeric reverse transcriptase from Moloney murine leukemia virus. Structure 12, 819-829 (2004).
10. Yu, S. F., Baldwin. D. N., Gwynn, S. R., Yendapalli, S. & Linial, M. L. Human foamy virus replication: a pathway distinct from that of retroviruses and hepadnaviruses. Science 271, 1579-1582 (1996).
11. Wohrl. B. M. Structural and functional aspects of foamy virus protease-reverse transcriptase. Viruses 11 (2019).
12. Lee, Y. N. & Bieniasz, P. D. Reconstitution of an infectious human endogenous retrovirus. PLoS Pathog 3, e10 (2007).
13. Mills, D. A., McKay, L. L. & Dunny, G. M. Splicing of a group II intron involved in the conjugative transfer of pRS01 in lactococci. J Bacteriol 178, 3531-3538 (1996).
14. Mohr. S. et al. Thermostable group II intron reverse transcriptase fusion proteins and their use in cDNA synthesis and next-generation RNA sequencing. RNA 19, 958-970 (2013).
15. Dai, L. & Zimmerly, S. ORF-less and reverse-transcriptase-encoding group II introns in archaebacteria, with a pattern of homing into related group II intron ORFs. RNA 9, 14-19 (2003).
16. Blocker, F. J. et al. Domain structure and three-dimensional model of a group II intron-encoded reverse transcriptase. RNA 11, 14-28 (2005).
17. Stamos, J. L., Lentzsch, A. M. & Lambowitz, A. M. Structure of a thermostable group II intron reverse transcriptase with template-primer and Its functional and Evolutionary implications. Mol Cell 68, 926-939 e924 (2017).
18. Zhao, C. & Pyle, A. M. Crystal structures of a group II intron maturase reveal a missing link in spliceosome evolution. Nat Struct Mol Biol 23, 558-565 (2016).
19. Zhao, C., Liu, F. & Pyle, A. M. An ultraprocessive, accurate reverse transcriptase encoded by a metazoan group II intron. RNA 24, 183-195 (2018).
20. Kelley, L. A., Mezulis, S., Yates, C. M., Wass, M. N. & Steinberg, M. J. The Phyre2 web portal for protein modeling, prediction and analysis. Nat Protoc 10, 845-858 (2015).
21. Truong, D. J. et al. Development of an intein-mediated split-Cas9 system for gene therapy. Nucleic Acids Res 43, 6450-6458 (2015).
22. Levy, J. M. et al. Cytosine and adenine base editing of the brain, liver, retina, heart and skeletal muscle of mice via adeno-associated viruses. Nat Biomed Eng 4, 97-110 (2020).
23. Petri, K. et al. CRISPR prime editing with ribonucleoprotein complexes in zebrafish and primary human cells. Nat Biotechnol (2021).
24. Hopp, T. P. et al. A short polypeptide marker sequence useful for recombinant protein identification and purification. BioTechnology 6, 1204-1210 (1988).
25. Hsu, J. Y. et al. PrimeDesign software for rapid and simplified design of prime editing guide RNAs. Nat Commun 12, 1034 (2021).
26. Rohland, N. & Reich, D. Cost-effective, high-throughput DNA sequencing libraries for multiplexed target capture. Genome Res 22, 939-946 (2012).
27. Gibson, D. G. et al. Enzymatic assembly of DNA molecules up to several hundred kilobases. Nat Methods 6, 343-345 (2009).
28. Kleinstiver, B. P. et al. Engineered CRISPR-Cas9 nucleases with altered PAM specificities. Nature 523, 481485 (2015).
29. Clement, K. et al. CRISPResso2 provides accurate and rapid genome editing sequence analysis. Nat Biotechnol 37, 224-226 (2019).
30. Smirkhina, S. A. Prime Editing: Making the Move to Prime Time. The CRISPR Journal 3(5): 319-321 (October 2020).
31. Scholefield, J. and Harrison, P. T. Prime editing—an update on the field. Gene Therapy 28:396-401 (2021).
32. Kim et al, Increasing the genome-targeting scope and precision of base editing with engineered Cas9-cytidine deaminase fusions. Nat Biotechnol. 35(4): 371-376 (2017).
33. Yang et al., Increasing targeting scope of adenosine base editors in mouse and rat embryos through fusion of TadA deaminase with Cas9 variants. Protein Cell. 2018 September; 9(9): 814-819
34. Richter et al., Phage-assisted evolution of an adenine base editor with improved Cas domain compatibility and activity. Nat Biotechnol. 2020 July; 38(7): 883-891
35. Chen, P. J. et al. Enhanced prime editing systems by manipulating cellular determinants of editing outcomes. Cell 184, 5635-5652 e5629 (2021)
36. Gramlich, M. et al. Antisense-mediated exon skipping: a therapeutic strategy for titin-based dilated cardiomyopathy. EMBO Mol Med 7, 562-576 (2015).
37. Tsai, S. Q. et al. GUIDE-seq enables genome-wide profiling of off-target cleavage by CRISPR-Cas nucleases. Nat Biotechnol 33, 187-197 (2015).
38. Kleinstiver, B. P. et al. High-fidelity CRISPR-Cas9 nucleases with no detectable genome-wide off-target effects. Nature 529, 490-495 (2016).
39. Bock, D. et al. In vivo prime editing of a metabolic liver disease in mice. Sci Transl Med 14, eabl9238 (2022).
40. Liu, P. et al. Improved prime editors enable pathogenic allele correction and cancer modelling in adult mice. Nat Commun 12, 2121 (2021)

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising:
(a) a Cas nickase protein and a reverse transcriptase (RT) protein, wherein the Cas nickase and RT are separate molecules, i.e., are not tethered, conjugated, or fused together, or
(b) a fusion protein comprising a Cas nickase protein linked to a reverse transcriptase (RT) protein, with a cleavable linker between the Cas nickase and the RT, optionally wherein the cleavable linker is a 2A self-cleaving peptide or protease-cleavable linker.

2. A composition comprising:
(a) a nucleic acid comprising (i) a sequence encoding a Cas nickase protein and (ii) a nucleic acid comprising a sequence encoding a reverse transcriptase (RT) protein, wherein the Cas nickase and RT are encoded as separate molecules, i.e., are not tethered, conjugated, or fused together, optionally wherein each nucleic acid is in a separate expression vector, e.g., a viral vector, e.g., an AAV, and/or
(b) a nucleic acid comprising a sequence encoding a Cas nickase protein in frame with a reverse transcriptase (RT) protein, with a cleavable linker between the Cas nickase and the RT, optionally wherein the cleavable linker is a 2A self-cleaving peptide or protease-cleavable linker, optionally wherein the nucleic acid is in an expression vector, e.g., a viral vector, e.g., an AAV.

3. The composition of claim 1 or 2, further comprising a pegRNA that can coordinate with the Cas nickase and RT to edit target DNA.

4. A method of editing target DNA, e.g., genomic DNA of a cell or DNA in vitro, the method comprising contacting the DNA or cell with, or expressing in the cell:
(a) both of (i) a Cas nickase protein and (ii) a reverse transcriptase (RT) protein and a pegRNA that can coordinate with the Cas nickase and RT to edit the target DNA, and optionally an ngRNA, wherein the Cas nickase and RT are separate molecules, i.e., are not tethered, conjugated, or fused together, and/or
(b) a fusion protein comprising a Cas nickase protein linked to a reverse transcriptase (RT) protein, with a cleavable linker between the Cas nickase and the RT, optionally wherein the cleavable linker is a 2A self-cleaving peptide or protease-cleavable linker, and a pegRNA that can coordinate with the Cas nickase and RT to edit the target DNA.

5. A truncated variant Moloney Murine Leukemia Virus reverse transcriptase (MMLV-RT) protein lacking any RNase H domain, preferably comprising a deletion of at least 1 and up to 207, 205, 200, 198, 195, 190, 185, or 181 amino acids from the C terminus, and optionally at least 1 and up to 23, 24, or 25 amino acids from the N terminus, and optionally wherein the MMLV-RT comprises mutations D200N/T330P/T306K/W313F and optionally L603W in MMLV-RT.

6. An isolated nucleic acid encoding the truncated variant MMLV-RT of claim 5, optionally wherein the nucleic acid is in an expression vector, e.g., a viral vector, e.g., an AAV.

7. A method of editing target DNA, e.g., genomic DNA of a cell or DNA in vitro, the method comprising contacting the DNA or cell with, or expressing in the cell, (i) a Cas nickase protein and (ii) truncated variant MMLV-RT protein of claim 5, and a pegRNA that can coordinate with the Cas nickase and RT to edit the target DNA, and optionally an ngRNA, wherein the Cas nickase and RT are separate molecules, or wherein the Cas nickase and RT are tethered, conjugated, or fused together, e.g., wherein the RT is fused to the Cas nickase at the N terminus or C terminus, optionally with a cleavable linker therebetween, optionally wherein the cleavable linker is a 2A self-cleaving peptide or protease-cleavable linker, or is inlaid internally.

8. A variant Eubacterium rectale reverse transcripase (MarathonRT) protein comprising a mutation as shown in Table C, preferably wherein the variant has increased prime editing efficiency compared to WT Marathon-RT, preferably wherein the variant comprises mutations at one, two, three, four, or all five of D14, N26, D74, N116, and/or N197, preferably D14R-N26R-D74R-N116K; D14R-D74R-N116K-N197R: D14R-N26R-D74R-N197R; or D14R-N26R-D74R-N116K-N197R.

9. An isolated nucleic acid encoding the variant MarathonRT of claim 8, optionally wherein the nucleic acid is in an expression vector, e.g., a viral vector, e.g., an AAV.

10. A method of editing target DNA, e.g., genomic DNA of a cell or DNA in vitro, the method comprising contacting the DNA or cell with, or expressing in the cell, (i) a Cas nickase protein and (ii) variant MarathonRT protein of claim 8, and a pegRNA that can coordinate with the Cas nickase and RT to edit the target DNA, and optionally an ngRNA, wherein the Cas nickase and RT are separate molecules, or wherein the Cas nickase and RT are tethered, conjugated, or fused together, e.g., wherein RT is fused to the Cas nickase at the N terminus or C terminus, optionally with a cleavable linker therebetween, optionally wherein the cleavable linker is a 2A self-cleaving peptide or protease-cleavable linker, or is inlaid internally.

11. A prime editor fusion protein comprising:
(i) a Cas9 nickase protein tethered, conjugated, or fused to the truncated variant MMLV-RT of claim 5, the variant MarathonRT protein of claim 8, or a wild type RT selected from MarathonRT, Human Endogenous Retrovirus K consensus sequence (HERV-Kcon RT), and Geobacillus stearothermophilus GsI-IIC intron RT (GsI-IIC RT), or
(ii) a Cas9 nickase protein comprising the truncated variant MMLV-RT of claim 5, the variant MarathonRT protein of claim 8, a MMLV-RT pentamutant or Geobacillus stearothermophilus GsI-IIC intron RT (GsI-IC RT) pentamutant, or a wild type RT selected from MarathonRT, Human Endogenous Retrovirus K consensus sequence (HERV-Kcon RT), and Geobacillus stearothermophilus GsI-IIC intron RT (GsI-IIC RI), wherein the MMLV-RT is inlaid into the Cas9 nickase, optionally wherein the MMLV is inlaid at G1247 or G1055.

12. A nucleic acid encoding the prime editor fusion protein of claim 11, optionally wherein the nucleic acid is in an expression vector, e.g., a viral vector, e.g., an AAV.

13. A composition comprising the prime editor fusion protein of claim 11, a nucleic acid encoding the prime editor fusion protein of claim 11, and a pegRNA, and optionally an ngRNA.

14. A composition comprising: (i) a Cas9 nickase protein and (ii) an RT, wherein the RT comprises the truncated variant MMLV-RT of claim 5, a MMLV-RT pentamutant or GsI-IIC RT pentamutant, the variant MarathonRT protein of claim 8, or a wild type RT selected from MarathonRT, Human Endogenous Retrovirus K consensus sequence (HERV-Kcon RT), and GsI-IIC RT, wherein the Cas nickase and RT are separate molecules, i.e., are not tethered, conjugated, or fused together, or wherein the Cas nickase and RT are tethered, conjugated, or fused together, optionally with a cleavable linker therebetween, optionally wherein the cleavable linker is a 2A self-cleaving peptide or protease-cleavable linker.

15. A composition comprising (i) a nucleic acid comprising a sequence encoding a Cas nickase protein and (ii) a nucleic acid comprising a sequence encoding an RT, wherein the RT comprises the truncated variant MMLV-RT of claim 5, the variant MarathonRT protein of claim 8, a MMLV-RT pentamutant or GsI-IIC RT pentamutant, or a wild type RT selected from MarathonRT, Human Endogenous Retrovirus K consensus sequence (HERV-Kcon RT), and GsI-IIC RT, wherein the Cas nickase and RT are encoded as separate molecules, i.e., are not tethered, conjugated, or fused together, optionally wherein each nucleic acid is in a separate expression vector, e.g., a viral vector, e.g., an AAV, or wherein the Cas nickase and RT are tethered, conjugated, or fused together, optionally with a cleavable linker therebetween, optionally wherein the cleavable linker is a 2A self-cleaving peptide or protease-cleavable linker.

16. A method of editing target DNA, e.g., genomic DNA of a cell or DNA in vitro, the method comprising contacting the DNA or cell with, or expressing in the cell, (i) a Cas nickase protein and (ii) an RT, wherein the RT comprises the truncated variant MMLV-RT of claim 5, a MMLV-RT pentamutant or GsI-IIC RT pentamutant, the variant MarathonRT protein of claim 8, or a wild type RT selected from MarathonRT, Human Endogenous Retrovirus K consensus sequence (HERV-Kcon RT), and GsI-IIC RT, wherein the Cas nickase and RT are separate molecules, i.e., are not tethered, conjugated, or fused together, and a pegRNA that can coordinate with the Cas nickase and RT to edit the target DNA, and optionally an ngRNA, wherein the Cas nickase and RT are separate molecules, or wherein the Cas nickase and RT are tethered, conjugated, or fused together, e.g., wherein RT is fused to the Cas nickase at the N terminus or C terminus, optionally with a cleavable linker therebetween, optionally wherein the cleavable linker is a 2A self-cleaving peptide or protease-cleavable linker, or is inlaid internally.

17. Any of the preceding claims, wherein the Cas nickase is a nickase shown in Table A1, or a variant thereof, e.g., as shown in Table A2, e.g., wherein the Cas nickase is Cas9, preferably from S. pyogenes (nSpCas9, e.g., comprising mutations H840, D839A, or N863A) or S. aureus (nSaCas9, e.g. comprising mutations D10A or N580).

18. Any of the preceding claims, wherein the Cas nickase is nSaCas9.

19. A method of transcribing RNA into DNA in vitro or in a cell, the method comprising contacting the RNA with an RT, wherein the RT comprises the truncated variant MMLV-RT of claim 5, a GsI-IIC RT pentamutant, the variant MarathonRT protein of claim 8, and nucleotides.

20. The method of claim 19, wherein the RNA is in a cell, and the method further comprises expressing the RT in the cell.


SEQUENCE LISTING (165 sequences)

SEQ ID NO: 1 - protein, 420 aa, Geobacillus stearothermophilus
MALLERILARDNLITALKRVEANQGAPGIDGVSTDQLRDYIRAHWSTIHAQLLAGTYRPAPVRRVEIPKPGGGTRQLGIPTVVDRLIQQAILQELTPIFDPDFSSSSFGFRPGRNAHDAVRQAQGYIQEGYRYVVDMDLEKFFDRVNHDILMSRVARKVKDKRVLKLIRAYLQAGVMIEGVKVQTEEGTPQGGPLSPLLANILLDDLDKELEKRGLKFCRYADDCNIYVKSLRAGQRVKQSIQRFLEKTLKLKVNEEKSAVDRPWKRAFLGFSFTPERKARIRLAPRSIQRLKQRIRQLTNPNWSISMPERIHRVNQYVMGWIGYFRLVETPSVLQTIEGWIRRRLRLCQWLQWKRVRTRIRELRALGLKETAVMEIANTRKGAWRTTKTPQLHQALGKTYWTAQGLKSLTQRYFELRQG

SEQ ID NO: 2 - protein, 599 aa, Lactococcus lactis
MKPTMAILERISKNSQENIDEVFTRLYRYLLRPDIYYVAYQNLYSNKGASTKGILDDTADGFSEEKIKKIIQSLKDGTYYPQPVRRMYIAKKNSKKMRPLGIPTFTDKLIQEAVRIILESIYEPVFEDVSHGFRPQRSCHTALKTIKREFGGARWFVEGDIKGCFDNIDHVTLIGLINLKIKDMKMSQLIYKFLKAGYLENWQYHKTYSGTPQGGILSPLLANIYLHELDKFVLQLKMKFDRESPERITPEYRELHNEIKRISHRLKKLEGEEKAKVLLEYQEKRKRLPTLPCTSQTNKVLKYVRYADDFIISVKGSKEDCQWIKEQLKLFIHNKLKMELSEEKTLITHSSQPARFLGYDIRVRRSGTIKRSGKVKKRTLNGSVELLIPLQDKIRQFIFDKKIAIQKKDSSWFPVHRKYLIRSTDLEIITIYNSELRGICNYYGLASNFNQLNYFAYLMEYSCLKTIASKHKGTLSKTISMFKDGSGSWGIPYEIKQGKQRRYFANFSECKSPYQFTDEISQAPVLYGYARNTLENRLKAKCCELCGTSDENTSYEIHHVNKVKNLKGKEKWEMAMIAKQRKTLVVCFHCHRHVIHKHK

SEQ ID NO: 3 - protein, 562 aa, unidentified
  source 1..562: note = Thermosynechococcus elongatus
METRQMAVEQTTGAVTNQTETSWHSIDWAKANREVKRLQVRIAKAVKEGRWGKVKALQWLLTHSFYGKALAVKRVTDNSGSKTPGVDGITWSTQEQKAQAIKSLRRRGYKPQPLRRVYIPKANGKQRPLGIPTMKDRAMQALYALALEPVAETTADRNSYGFRRGRCIADAATQCHITLAKTDRAQYVLDADIAGCFDNISHEWLLANIPLDKRILRKWLKSGFVWKQQLFPIHAGTPQGGVISPMLANMTLDGMEELLNKFPRAHKVKLIRYADDFVVTGETKEVLYIAGAVIQAFLKERGLTLSKEKTKIVHIEEGFDFLGWNIRKYDGKLLIKPAKKNVKAFLKKIRDTLRELRTAPQEIVIDTLNPIIRGWTNYHKNQASKETFVGVDHLIWQKLWRWARRRHPSKSVRWVKSKYFIQIGNRKWMFGIWTKDKNGDPWAKHLIKASEIRIQRRGKIKADANPFLPEWAEYFEQRKKLKEAPAQYRRTRRELWKKQGGICPVCGGEIEQDMLTEIHHILPKHKGGTDDLDNLVLIHTNCHKQVHNRDGQHSRFLLKEGL

SEQ ID NO: 4 - protein, 419 aa, Sinorhizobium sp.
MTSESTTDKPFRIEKRRVYEAYKAVKANRGAAGVDGQTLEIFEKDLAANLYKIWNRMSSGTYFPPPVRAVSIPKKAGGERVLGVPTVSDRIAQMVVKQMIEPDLDSLFLPDSYGYRPGKSALDAVGVTRQRCWKYDWVLEFDIKGLFDNLPHDLLLKAVRKDVKCNWALLYIERWLTAPMEKNGEVIERSRGTPQGGVVSPILANLFLHYAFDLWMTRTHPDLPWCRYADDGLVHCQSEQQAEALKVELSSRLAACGLQMHPTKTKIVYCKDQRRREAYPNVTFDFLGYQFRPRRVANTQWDEFFCGYTPAVSPTALKSMRATIKSLNIPRQTPGTLAEIAKQLNPLLRGWIAYYGRYSRSALSTLADYVNQKLRAWIRRKFKRFQSHKTRASLFLRKLARENPGLFVHWKAFGTNTFT

SEQ ID NO: 5 - protein, 415 aa, Methanosarcina acetivorans
MDETKPYEISKDIVQEAFQRVKANKGAAGVDDENIAAFESDLTNNLYKIWNRMSSGCYFPPSVKAIEIPKKSGGTRILGIPTVLDRVAQMVTKIYLEPQLEPLFHPDSYGYRPGKSAADALAATRKRCWRYNWLLEFDIKGLFDNINHDLLMKQVSMHTDKPWIILYIQRWLKAPFQMADGTVNERTKGTPQGGVVSPLLANLFLHYAFDQWMDSHHRYNPFERYADDSVIHCRSREEAERLWIELDKRLSEFGLELHPSKTRIVYCKDDDRQGDYPETKFDFLGYTFRPRRSKNKYGKHFINFTPAVSNTAKKSMQQEIHDWRMHLKPDKTLEDLSHMFNPILRGWVNYYGLFYKSELYCVLKHMNRVLTRWAQRKYKKLAGHKRRARYWLGKIARRDPKLFVHWQMGIFPEAG

SEQ ID NO: 6 - protein, 434 aa, Enterobacter cloacae
MRPLPQAVDEIQHHEVQNQPPRNPTSWMAQVLARDNLIRALNQVKRNKGAAGVDGMTVERLSDYLKQHWPALKEQLETGNYQPEAVKRVEIPKADGRKRKLGIPTVLDRFIQQAIAQVLSQHWESQFHNNSYGFRPMRSAHQAVSYAKALLLSGKGWVVDLDLDAFFDRVNHDRLMSKLRAQIQDPTLLKLIQRYLKANIDHNGKQEACREGVPQGGPLSPLLANIVLNELDWELERRGHSFARYADDCQIYTSSKRAGERIKQSIERYIETRLRLKVNKAKSAVARPWERSFLGFTFSRRKGNRLKVTDKALDRLKDKLRELTRRTRGHNIGSVIADIRKALLGWKAYFGIAEVQSQLRDTDKWLRRKLRCYIWKQWGSKGYRMLRKAGVDRFLAWNTAKSAHGPWRLSKSPALYIALPNRYFTNMGLPTIAA

SEQ ID NO: 7 - protein, 470 aa, Clostridium acetobutylicum
MKNSKEMQKLQTTSYKEGWSCEIRVELQNSTRAHSISTAFDRRKDDGKLYETNLLERILDRQNMNLAYKRVKSNKGSHGVDGMKVDELLQYLKQNGKTLIASIFNGKYCPKAVRRVEIPKPDGGIRLLGIPTVVDRTIQQAISQVLTPIFEKTFSENSYGFRPKRSAKQAIKKAKEYMEEGYKWVVDIDLAKYFDTVNHDKLMALVARKIKDKRVLKLIRLYLQSGVMINGVVSETERGCPQGGPLSPLLSNIMLTELDRELEKRGHKFCRYADDNNVYVRSKKAGDRVMRSITRFIENKLKLKVNKEKSAVDRPWRRKFLGFTFYQWYGKIGIRVHEKSVKKFKAKIKAITARSNALNIENRIIKLRQCIIGWLNYFGIAEMTKLAKKLDEWTRRRLRMCYWKQWKKVKTKYDNLRKFGINNSKAWEFANTRKSYWRIANSPILSTTLTNSYLEKIGYTSIFKRYKQVH

SEQ ID NO: 8 - protein, 418 aa, Bacillus halodurans
MLERILSRENLIQALERVEKNKGSYGVDEMDVKSLRLHLHENWTSIRNEIIEGSYFPKPVRRVEIPKPNGGVRKLGIPTVMDRFLQQAIAQILTQLYDPTFSERSFGFRPHRRGHNAVRQAKQWMKEGYRWVVDIDLEKFFDKVNHDRLMRKLSSRIQDPRVLQLIRRYLQTGVMERGLVSPNTEGTPQGGPLSPLLSNIVLDELDNELEKRGLKFVRYADDCNIYVRSKRAGLRIMESVTSFIENRLKLKVNREKSAVDRPWNRKFLGFSFTRGKDPKMRVSKESVKRLKQRIRELTSRRHSMKMSDRLRRLNRYLTGWLGYYQLVDTPSILAQIDAWIRRRLRMIRWKEWKTTSARQKNLVRLGIKKAKAWQWANSRKGYWRVAHSPIMDYALNSEYWKGQGLMSLAERYQTRRWT

SEQ ID NO: 9 - protein, 490 aa, Pseudomonas alcaligenes
MPPVGVAVSLVTVMQKFPTAETVIPNPGQKPRVMPDSAKVPAASATWTNAEPDTLMERVLAPANLRRAYQRVVSNKGAPGADGMTVADLAGYVKQYWPTLKARLLAGEYHPQAVRAVEIPKPQGGTRQLGIPSVVDRLIQQALQQQLTPIFDPLFSDYSYGFRPGRSTHQAIEMARAHVTAGHRWCVELDLEKFFDRVNHDILMACIERRIKDKCVLRLIRRYLEAGIMSGGVVSPRQEGTPQGGPLSPLLSNILLDELDRELERRGHRFVRYADDANIYVRSPRAGERVLVSVERFLRERLKLTVNRKKSQVARAWKCDYLGYGMSWHQQPRLRVARMSLDRLRDRLRMLLRSVRARKMATVIERINPVLRGWASYFKLSQSKRPLEELDGWVRHKLRCVIWRQWKQPPTRLRNLMRLGLSEERANKSAFNGRGPWWNSGAQHMNYALPKKLWDRLGLVSILDTINRLSRNLNRRVRNRTHGGVRGRRV

SEQ ID NO: 10 - protein, 494 aa, Pseudomonas putida
MTVIGSAAKTDAIGTGAPSHAERMWLQANWGLIKEDVKRLQARIAKATMEGRWGKVKALQHLLTRSHNGKMLAVKRVTENRGKRTPGVDGKIWATPAAKSSGMESMRHRSYRALPLRRIYIPKSNGQKRPLGIPRMLCRSMQALWKLALEPVSESLADPNSYGFRPNRSTADAIEYCFITLAKRTSPVWVLEGDIRGCFDNFNHEWMLKNIPMDKTILRRWLQAGFIDEGTLFATQAGTPQGGIISPVIANMALDGLEAAVHASVGPTKRARERSKINVVRYADDFVVTGISKEILEHSVLPAVRQFMAIRGLELSEEKTKITHIAEGFDFLGQNVRKYQGKLLIKPANKSVKALLDKVREIVKSNKSATQANLILQLNPIIRGWAMYHRHVVSKSLFSSIDAQIWRLLWTWALRRHPNKGAGWVRQRYFHTVRYQNWVFRAQTKVGGIVQRWWLFRASTIPIVRHVKIRGLANPFDPAWSSYFARRRSAMDVD

SEQ ID NO: 11 - protein, 436 aa, Streptococcus agalactiae
MQTTKKERNTHMSELLDKISSRNNMLEAYKQVKSNKGSAGIDGVTIEQMDDYLHQNWRETKKLIKERSYKPQPVLRVEIPKPNGGVRNLGIPTAMDRMIQQAIVQVLSPLCEKHFSEYSYGFRPNRSCETAIVQLLEYLNDGYEWIVDIDLEKFFDTVPQDRLMSLVHNIIQDGDTESLIRKYFHSGVVINGQRHKTLVGTPQGGNLSPLLSNIMLNELDKGLEKRGLRFVRYADDCVITVGSEAAAKRVMHSVSSYIEKRLGLKVNMTKTKIVRPNKLKYLGFGFWKSPKGWKCRPHQDSVQSFKRKLKQLTMRKWSIDLITRIERLNWVIRGWINYFSLGNMKSIMTQIDERLRTRIRVIIWKQWKKKAKRLWGLLKLGVARWIADKVSGWGDHYQLVAQKSVLTRAISKPALAKRGLVSCLDYYLERHALKVS

SEQ ID NO: 12 - protein, 439 aa, Roseburia intestinalis
MVKSSGTERKERMDTSSLMEQILSNDNLNRAYLQVVRNKGAEGVDGMKYTELKEYLAKNGEIIKEQLRIRKYKPQPVRRVEIPKPDGGVRNLGVPTVTDRFIQQAIAQVLTPIYEEQFHDHSYGFRPNRCAQQAILTALDMMNDGNDWIVDIDLEKFFDTVNHDKLMTIIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPRGIKYLGFGFYYDTSAQQFKAKPHAKSVMKYKKRMRELTCRSWGVSNSYKVERLNQLIRGWINYFKIGSMKTLCRELDGNIRYRIRMCIWKHWKTPQNKEKNLVKLGVPRWAAHKVANTGNRYAHMCHNGWIQKAISTKRLTSFGLVSMLDYYTERCVTC

SEQ ID NO: 13 - protein, 427 aa, Eubacterium rectale
MDTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTC

SEQ ID NO: 14 - protein, 552 aa, Streptococcus sp.
MNSKMCATTNIANSWESIDFVKAEIYVKKLQMRIVKAWKLGKFNRVKSLQHLLTTSFYARALAVKRVTENQGKKTSGVDKELWLTPNAKYQAIKKLKVRGYCPKPLRRIYIPKKNGKKRPLSIPTMTDRAMQTLFKFALEPIAETTADPNSYGFRPKRSTQDAIEQCFLALSKQKSAKWVLEGDIKGCFDNISHEWIMKNIPMNKTILGKWLKSGYIENQKLFPTELGSPQGSPISPIISNMVLDGLERKLSATFRKKKVNGKVYTPKINFVRYADDFIVTGVSKELLENEVKPVIIEFLKERGLELSEEKTLITHITDGFDFLGINIRMYEGKLLTKPSKKNYESIASKIREVIKQNPSMKQELLIRKLNPSIIGWVNYQKHNVSTEAFQRLDNDIYQCLWRWCIRRHPKKGRKWVANKYFHTFGSRSWIFSVQTTDTMENGEPFYLRLRCASDTDIRRHIKVKAEANPFDEQWQLYFEERQEKQMRQELKGRRVINGLYYKQKGVCPVCESKITKETDFRVHQTVKNHKPIKTLVHPTCHKNIKENTLVL

SEQ ID NO: 15 - protein, 555 aa, Shigella sonnei
MNTHISVSTIPHLTGWHAINWKACHARVRKLQLRIAKATRQQQWRQVRELQRILTRSFSGKAVAVRRVTENTGKRTPGIDGKIWHTPKEKWGGVCSLNLRGYRPQPLRRIHIPKSNGKTRPLGIPTMRDRAMQALWLLALEPVSETTADHNSYGFRPMRSTHDAIESIFLRMSQKVSPKWILEGDIKGCFDNISHDWLLSHIPMDRRLLKKWLKAGYMERGVFNHTNSGTPQGGIISPVLANMALDGLEKELMQTFRKSGYHSAKHQVNYVRYADDFICSGSSRELLENEVRPLIAAFMRERGLELSEEKTAITHIDKGFDFLGQNVRKYNGKMLIKPSKKNLKNFLCKVREIIKRNPTLPAWKLIGQLNPVIRGWATYHRHVVAKETFNYVDTQIWRAIWRWCVRRHPRKGLRWIAGRYFSFEGRRWIFKAITPEGKILTLFRAMETPIKRHIKIKGEATPYTPGMEIYFERRLDLIWKGKSKKMKTVVQLWKRQGKHCPQCGQPITNQTGWNIHHRIRKVMGGSDELTNLELLHPNCHRQLHSREAGAHRKHL

SEQ ID NO: 16 - protein, 834 aa, Saccharomyces cerevisiae
MVQRWLYSTNAKDIAVLYFMLAIFSGMAGTAMSLIIRLELAAPGSQYLHGNSQLFNGAPTSAYISLMRTALVLWIINRYLKHMTNSVGANFTGTMACHKTPMISVGGVKCYMVRLTNFLQVFIRITISSYHLDMVKQVWLFYVEVIRLWFIVLDSTGSVKKMKDTNNTKGNTKSEGSTERGNSGVDRGMVVPNTQMKMRFLNQVRYYSVNNNLKMGKDTNIELSKDTSTSDLLEFEKLVMDNMNEENMNNNLLSIMKNVDMLMLAYNRIKSKPGNMTPGTTLETLDGMNMMYLNKLSNELGTGKFKFKPMRMVNIPKPKGGMRPLSVGNPRDKIVQEVMRMILDTIFDKKMSTHSHGFRKNMSCQTAIWEVRNMFGGSNWFIEVDLKKCFDTISHDLIIKELKRYISDKGFIDLVYKLLRAGYIDEKGTYHKPMLGLPQGSLISPILCNIVMTLVDNWLEDYINLYNKGKVKKQHPTYKKLSRMIAKAKMFSTRLKLHKERAKGPTFIYNDPNFKRMKYVRYADDILIGVLGSKNDCKMIKRDLNNFLNSLGLTMNEEKTLITCATETPARFLGYNISITPLKRMPTVTKTIRGKTIRSRNTTRPIINAPIRDIINKLATNGYCKHNKNGRMGVPTRVGRWTYEEPRTIINNYKALGRGILNYYKLATNYKRLRERIYYVLYYSCVLTLASKYRLKTMSKTIKKFGYNLNIIENDKLIANFPRNTFDNIKKIENHGMFMYMSEAKVTDPFEYIDSIKYMLPTAKANFNKPCSICNSTIDVEMHHVKQLHRGMLKATKDYITGRMITMNRKQIPLCKQCHIKTHKNKFKNMGPGM

SEQ ID NO: 17 - protein, 854 aa, Saccharomyces cerevisiae
MVQRWLYSTNAKDIAVLYFMLAIFSGMAGTAMSLIIRLELAAPGSQYLHGNSQLFNVLVVGHAVLMIFCAPFRLIYHCIEVLIDKHISVYSINENFTVSFWFWLLVVTYMVFRYVNHMAYPVGANSTGTMACHKSAGVKQPAQGKNCPMARLTNSCKECLGFSLTPSHLGIVIHAYVLEEEVHELTKNESLALSKSWHLEGCTSSNGKLRNTGLSERGNPGDNGVFMVPKFNLNKVRYFSTLSKLNARKEDSLAYLTKINTTDFSELNKLMENNHNKTETINTRILKLMSDIRMLLIAYNKIKSKKGNMSKGSNNITLDGINISYLNKLSKDINTNMFKFSPVRRVEIPKTSGGFRPLSVGNPREKIVQESMRMMLEIIYNNSFSYYSHGFRPNLSCLTAIIQCKNYMQYCNWFIKVDLNKCFDTIPHNMLINVLNERIKDKGFMDLLYKLLRAGYVDKNNNYHNTTLGIPQGSVVSPILCNIFLDKLDKYLENKFENEFNTGNMSNRGRNPIYNSLSSKIYRCKLLSEKLKLIRLRDHYQRNMGSDKSFKRAYFVRYADDIIIGVMGSHNDCKNILNDINNFLKENLGMSINMDKSVIKHSKEGVSFLGYDVKVTPWEKRPYRMIKKGDNFIRVRHHTSLVVNAPIRSIVMKLNKHGYCSHGILGKPRGVGRLIHEEMKTILMHYLAVGRGIMNYYRLATNFTTLRGRITYILFYSCCLTLARKFKLNTVKKVILKFGKVLVDPHSKVSFSIDDFKIRHKMNMTDSNYTPDEILDRYKYMLPRSLSLFSGICQICGSKHDLEVHHVRTLNNAANKIKDDYLLGRMIKMNRKQITICKTCHFKVHQGKYNGPGL

SEQ ID NO: 18 - protein, 328 aa, Bordetella sp.
MGKRHRNLIDQITTWENLLDAYRKTSHGKRRTWGYLEFKEYDLANLLALQAELKAGNYERGPYREFLVYEPKPRLISALEFKDRLVQHALCNIVAPIFEAGLLPYTYACRPDKGTHAGVCHVQAELRRTRATHFLKSDFSKFFPSIDRAALYAMIDKKIHCAATRRLLRVVLPDEGVGIPIGSLTSQLFANVYGGAVDRLLHDELKQRHWARYMDDIVVLGDDPEELRAVFYRLRDFASERLGLKISHWQVAPVSRGINFLGYRIWPTHKLLRKSSVKRAKRKVANFIKHGEDESLQRFLASWSGHAQWADTHNLFTWMEEQYGIACH

SEQ ID NO: 19 - protein, 352 aa, unidentified
  source 1..352: note = ANMV-1 virus
MNAQQDNPTAKMETYKHLYTQICTKENICKAYRKARLGKRKKFYVRKFESDVDANIEQLHQQLRDESWTPLPYKQFTAYEPKERLIRAPQFPDRIVHHALIRMLEPIYNKILIYDTYASRKNKGTHATVDRLTRFLRRDNDNVFVFHGDVRKFFDNIDHETLIKILRKKIVDERVITLIKKILTNQGISLGVTLGNYTSQWFANIYLSELDYFAKHNLKVKHYIRYMDDFLLLSDSKPELHRWKHQIEKFLNERLKLELHPVKRQIFPTNIGIDFVGYTIWKDHKKLRRRDVNRFISRLNEFDKLPVMTPFAEASLMSWKGYSIHADAFGLTKQLHKSHPAMQVSTLDRYIN

SEQ ID NO: 20 - protein, 434 aa, Bacteroides sp.
MRRVGYIIEEIVEPSNMEASFRQVLRGSKRKRSRQGCYLLAHKPEVLEELVAQIASGTFRVKDYREREIIEGGKLRRIQVIPMKDRIAVHAIMAVVDRHLRKRFIRTTSASIKRRGMHDLLAYVRRDMAEDPDGTRYCYKFDITKFYESVKQDFVMYCVSRVFKDAKLVTMLESFIRLMPEGLSIGLRSSQGLGNLLLSVYLDHYLKDRYAVRHFYRYCDDGVVLGKTKAELWKIRDAVHGRMECAGLLVKGNERVFPPGEGIDFLGYVTFGADHVRLRKRIKQKFARKMHEVKSRRRRRELIASFYGMAKHADCHTLFKKLTGKDMRSFKDLNVSYKPEDGKKRFPGVVVSIRELVNLPIVVKDFETGIKTEQGEDRCIVAIEMNGEPKKFFTNSEEMKNILLQVKDMPDGFPFETTIKTETFGKGRTKYIFT

SEQ ID NO: 21 - protein, 328 aa, Treponema denticola
MKRKGNLYHKITEWNNLIAAFYNASRGKRLKPDVLLYEKNLYTNLKTLQNYLINQTVLLGSYRFFKIYDPKERIICAAPFNERVLHHAIINITESVFEKFQIYDSYACRKNKGTQAALLRALYFSRRFKYFLKLDMKKYFDSIPHSKLSLLLTCKFKDKALLHLFNKLIASYSVTEGWGVPIGNLTSQYFANFYLSFFDHYAKEKMNVRGYIRYMDDVLLFSDNLKDIKLIQKKAKNFLSCELDLTLKEEIIGMVKNGIPFLGFLVKPQGIYLSQKKKKRLKKKIKDYVHKFKIAYWTEEEFALHITPVFAHIAISRCRAYCNKYLLT

SEQ ID NO: 22 - protein, 346 aa, unidentified
  source 1..346: note = archaeon GW2011_AR20
MQTYNKLFDKLCSYENLFLAYKKARKGKTGKGYVIKFEENLEDNLKILQFELINKIYKPKKLKLFIIRGPKTRRICKSAFRDRIVHHAIINILEPVYEKIFIHDSYASRKNKGQHRALERFDYFKRIASKNGKKLKGIRDKNYICGYCLKADIKKYFDNVNHETLINIIKKKIHDEDLIWLISQILGNKILGGDGKKGMPLGNYTSQFFANVYLNELDCFVKHNLKMRYYIRYVDDFVILYNDKETLEFYKREIDKFLKNKLKIELHEDKSKIIPLHKGIHFLGFRNFYYYRLLKKSNINQIRRNLKEWNEAYKNDDGNLKTRTKGWKAHAKHGNNYKLAKILLNA

SEQ ID NO: 23 - protein, 677 aa, unidentified
  source 1..677: note = Baboon endogenous virus strain M7
TVSLQDEHRLFDIPVTTSLPDVWLQDFPQAWAETGGLGRAKCQAPIIIDLKPTAVPVSIKQYPMSLEAHMGIRQHIIKFLELGVLRPCRSPWNTPLLPVKKPGTQDYRPVQDLREINKRTVDIHPTVPNPYNLLSTLKPDYSWYTVLDLKDAFFCLPLAPQSQELFAFEWKDPERGISGQLTWTRLPQGFKNSPTLFDEALHRDLTDFRTQHPEVTLLQYVDDLLLAAPTKKACTQGTRHLLQELGEKGYRASAKKAQICQTKVTYLGYILSEGKRWLTPGRIETVARIPPPRNPREVREFLGTAGFCRLWIPGFAELAAPLYALTKESTPFTWQTEHQLAFEALKKALLSAPALGLPDTSKPFTLFLDERQGIAKGVLTQKLGPWKRPVAYLSKKLDPVAAGWPPCLRIMAATAMLVKDSAKLTLGQPLTVITPHTLEAIVRQPPDRWITNARLTHYQALLLDTDRVQFGPPVTLNPATLLPVPENQPSPHDCRQVLAETHGTREDLKDQELPDADHTWYTDGSSYLDSGTRRAGAAVVDGHNTIWAQSLPPGTSAQKAELIALTKALELSKGKKANIYTDSRYAFATAHTHGSIYERRGLLTSEGKEIKNKAEIIALLKALFLPQEVAIIHCPGHQKGQDPVAVGNRQADRVARQAAMAEVLTLATEPDNTSHIT

SEQ ID NO: 24 - protein, 669 aa, Feline leukemia virus
TLQLEEEYRLFEPESTQKQEMDIWLKNFPQAWAETGGMGTAHCQAPVLIQLKATATPISIRQYPMPHEAYQGIKPHIRRMLDQGILKPCQSPWNTPLLPVKKPGTEDYRPVQDLREVNKRVEDIHPTVPNPYNLLSTLPPSHPWYTVLDLKDAFFCLRLHSESQLLFAFEWRDPEIGLSGQLTWTRLPQGFKNSPTLFDEALHSDLADFRVRYPALVLLQYVDDLLLAAATRTECLEGTKALLETLGNKGYRASAKKAQICLQEVTYLGYSLKDGQRWLTKARKEAILSIPVPKNSRQVREFLGTAGYCRLWIPGFAELAAPLYPLTRPGTLFQWGTEQQLAFEDIKKALLSSPALGLPDITKPFELFIDENSGFAKGVLVQKLGPWKRPVAYLSKKLDTVASGWPPCLRMVAAIAILVKDAGKLTLGQPLTILTSHPVEALVRQPPNKWLSNARMTHYQAMLLDAERVHFGPTVSLNPATLLPLPSGGNHHDCLQILAETHGTRPDLTDQPLPDADLTWYTDGSSFIRNGEREAGAAVTTESEVIWAAPLPPGTSAQRAELIALTQALKMAEGKKLTVYTDSRYAFATTHVHGEIYRRRGLLTSEGKEIKNKNEILALLEALFLPKRLSIIHCPGHQKGDSPQAKGNRLADDTAKKAATETHSSLTVL

SEQ ID NO: 25 - protein, 609 aa, unidentified
  source 1..609: note = Human foamy virus
NQVGHRKIRPHNIATGDYPPRPQKQYPINPKAKPSIQIVIDDLLKQGVLTPQNSTMNTPVYPVPKPDGRWRMVLDYREVNKTIPLTAAQNQHSAGILATIVRQKYKTTLDLANGFWAHPITPESYWLTAFTWQGKQYCWTRLPQGFLNSPALFTADVVDLLKEIPNVQVYVDDIYLSHDDPKEHVQQLEKVFQILLQAGYVVSLKKSEIGQKTVEFLGFNITKEGRGLTDTFKTKLLNITPPKDLKQLQSILGLLNFARNFIPNFAELVQPLYNLIASAKGKYIEWSEENTKQLNMVIEALNTASNLEERLPEQRLVIKVNTSPSAGYVRYYNETGKKPIMYLNYVFSKAELKFSMLEKLLTTMHKALIKAMDLAMGQEILVYSPIVSMTKIQKTPLPERKALPIRWITWMTYLEDPRIQFHYDKTLPELKHIPDVYTSSQSPVKHPSQYEGVFYTDGSAIKSPDPTKSNNAGMGIVHATYKPEYQVLNQWSIPLGNHTAQMAEIAAVEFACKKALKIPGPVLVITDSFYVAESANKELPYWKSNGFVNNKKKPLKHISKWKSIAECLSMKPDITIQHEKGISLQIPVFILKGNALADKLATQGSYVVN

SEQ ID NO: 26 - protein, 560 aa, Feline immunodeficiency virus
QISDKIPVVKVKMKDPNKGPQIKQWPLTNEKIEALTEIVERLEREGKVKRADPNNPWNTPVFAIKKKSGKWRMLIDFRELNKLTEKGAEVQLGLPHPAGLQIKKQVTVLDIGDAYFTIPLDPDYAPYTAFTLPRKNNAGPGRRFVWCSLPQGWILSPLIYQSTLDNIIQPFIRQNPQLDIYQYMDDIYIGSNLSKKEHKEKVEELRKLLLWWGFETPEDKLQEEPPYTWMGYELHPLTWTIQQKQLDIPEQPTLNELQKLAGKINWASQAIPDLSIKALTNMMRGNQNLNSTRQWTKEARLEVQKAKKAIEEQVQLGYYDPSKELYAKLSLVGPHQISYQVYQKDPEKILWYGKMSRQKKKAENTCDIALRACYKIREESIIRIGKEPRYEIPTSREAWESNLINSPYLKAPPPEVEYIHAALNIKRALSMIKDAPIPGAETWYIDGGRKLGKAAKAAYWTDTGKWQVMELEGSNQKAEIQALLLALKAGSEEMNIITDSQYVINIILQQPDMMEGIWQEVLEELEKKTAIFIDWVPGHKGIPGNEEVDKLCQTMMIIEG

SEQ ID NO: 27 - protein, 596 aa, unidentified
  source 1..596: note = human endogenous retrovirus K
KSRKRRNRVSFLGAATVEPPKPIPLTWKTEKPVWVNQWPLPKQKLEALHLLANEQLEKGHIEPSFSPWNSPVFVIQKKSGKWRMLTDLRAVNAVIQPMGPLQPGLPSPAMIPKDWPLIIIDLKDCFFTIPLAEQDCEKFAFTIPAINNKEPATRFQWKVLPQGMLNSPTICQTFVGRALQPVREKFSDCYIIHYIDDILCAAETKDKLIDCYTFLQAEVANAGLAIASDKIQTSTPFHYLGMQIENRKIKPQKIEIRKDTLKTLNDFQKLLGDINWIRPTLGIPTYAMSNLFSILRGDSDLNSKRMLTPEATKEIKLVEEKIQSAQINRIDPLAPLQLLIFATAHSPTGIIIQNTDLVEWSFLPHSTVKTFTLYLDQIATLIGQTRLRIIKLCGNDPDKIVVPLTKEQVRQAFINSGAWQIGLANFVGIIDNHYPKTKIFQFLKLTTWILPKITRREPLENALTVFTDGSSNGKAAYTGPKERVIKTPYQSAQRAELVAVITVLQDFDQPINIISDSAYVVQATRDVETALIKYSMDDQLNQLFNLLQQTVRKRNFPFYITHIRAHTNLPGPLTKANEQADLLVSSALIKAQELHA

SEQ ID NO: 28 - protein, 415 aa, Necator americanus
MDKAKPFSISKREVWEAYKQVKANRGAAGVDEQSMQEFEADLKNNLYRIWNRMSSGSYMPPPVLRVDIPKAGGAGTRSLGIPTISDRIAQTVVKRYLESLVEPVFHDDSYGYRPGRSAHRALDVARQRCWSYAWALDLDIKNFFGSIDWELMMRAVRRHTDCAWVLLYVERWLKARVQMPDGTVMQPDKGTPQGGVVSPVLANLFLHYALDRWMQTHHPDVPFERYADDAIYHCKSEEQARLLRQEVEVRLAECKLALHPEKTKIVYCKQANRPVDYPTCQFDFLGYTFRPRSVMNRMGKLSVGFTPAVSNKAAKAMRQELRRKPLWHRSDLTLNDLADYTRPILRGWIQYYGRFSRSVLAQVLRYVDAALVRWARRKYKSLSRRPARAWTWLSGIRSRQPGLFAHWSVEAAVGR

SEQ ID NO: 29 - protein, 739 aa, Axinella verrucosa
MRRLIWAGKGRRSTMDCYDVHMSTGLGRRESRLLNIASLFEAEGRQNACANRPRDIVPMAMAEWLKAILLLPSLDGGYLGRHGVSEMRRLLWICSRRVTRLAGDTISVHNEDNSRPKGTRPNPGNSGWPKGRNPYGHRAGVVQGPASPGRPAVSASLTSRHYSTGSAPKVVRRLKGLTERCINHPNLAVDRNIYPLLCDPYLLTVAYNNIRSKPGNMTPGVVPETLDGVSYETVKEISDGLRNETFQFKPGRKTQIPKQSGGLRSLTIAPPRDKIVQEAMRILLNDIFEPTFSDLSHGFRPGRSCHTALQMIQQRFKPVTWMIEGDISKCFDSIDHGLLMAIIEKKIKDRQFTKLIWKSLRAGYFEFHTIRHNIAGTPQGSIISPILSNIFMHQLDVFVEEMKAEFDRGSRARNTAEYEHRRYLMKRAKRLGNTGELARIYKEAKKNPVMDFRDPSYKRLAYVRYADDWVVGVRGSYKEAERTLDRITEFCRSISLTVSQSKTKITNLNKDKADFLGVNIFRSKHVKHSRKSSSAKQRQNLQLQFHVSIDRVRSKLSSASIIRNGVAAPRFLWLPLSHRQIISLYNSVLRGYLNYYCFVGNHSRLVGWLRWTIYTSAAMLLGRKYGLSTTKVFKRFGPRLSDGDTAGLHDPDYKATGKFRSKANPIVTGLYAKHVSIANLERLACEICGSGYRVEMHHVRHMKDLNPAASVVDRLMARANRKQIPLCRECHMKRHRGEI

SEQ ID NO: 30 - protein, 732 aa, Axinella verrucosa
MCIIVLILGICIKAVSLPIRGQLGGDNSMLAKGGWKSSPRAKVAMVRLTNPLTDEAGQKSRAAKRVVASNGIVCYIVVTIQRQSYARSASSILNIGEHLRSQYGMWWNSGNPESRKAGGFGGIVVLPSRGMATAGRKGSRSKVSKEPGLAGFGKLEKLCEQIKVKESKGIGGLTEIMADPRFLGTSYQKRRSMPGMMTPGTDKVTLDGISEKWFDEISQTFRNGLFKFRPVRRIGIPKPKGGVRYLGIPSPRDRIVQDAMKTLLELIFEPTFSDASHGYRPGRGCHTALNHIKMKMGYVTWFIEGDISKCFDSVNHRRLMGIIEEAVSDQPFMDLIHKALKAGYIEHPKGWVATNVGTPQGGVLSPLLANIYLDAFDKWMERKTESLEKGKRRRANPEYTKMIRESRVNREGYVAPLMGADENFKRVRYVRYADDFLIGVSGSLADCKNLRDEISEFLKRELELDLNLGKTRITHARSESAAFLGYRIHITDPSKYAQRYVLRKGRYKWTHISTRPKMDAPIEKLVEKLGEQKFCKPGGRPTSNGKFIHESLKEIIVRYRLLEKGLLNYYYMATNYGRVSARIHYTLKYSCALTIGRKMRLSTLKKVFKAYGKSLEVRDEKGRCIASYPKISYARPAGKISTAVVSPFDLIGNCAKFWKRSLDSRGLQCAVCQATEGIEMHHVKHLRKSKDMDWLTRRIVTMNRKQIPVCKECHQKIHRGRYDGRGLNRLIP

SEQ ID NO: 31 - protein, 785 aa, synthetic construct
MILDTDYITEDGKPVIRIFKKENGEFKIEYDRTFEPYLYALLKDDSAIEEVKKITAERHGTVVTVKRVEKVQKKFLGRPVEVWKLYFTHPQDVPAIMDKIREHPAVIDIYEYDIPFAIRYLIDKGLVPMEGDEELKLLAFDIETLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRHQSHEGGYIKEPERGLWENIVYLDFRSLYPSIIITHNVSPDTLNREGCKEYDVAPQVGHRFCKDFPGFIPSLLGDLLEERQKIKKRMKATIDPIERKLLDYRQRAIKILANSLYGYYGYARARWYCKECAESVIAWGREYLTMTIKEIEEKYGFKVIYSDTDGFFATIPGADAETVKKKAMEFLKYINAKLPGALELEYEGFYKRGLFVTKKKYAVIDEEGKITTRGLEIVRRDWSEIAKETQARVLEALLKDGDVEKAVRIVKEVTEKLSKYEVPPEKLVIHKQITRDLKDYKATGPHVAVAKRLAARGVKIRPGTVISYIVLKGSGRIVDRAIPFDEFDPTKHKYDAEYYIEKQVLPAVERILRAFGYRKEDLRYQKTRQVGLSARLKPKGTLEGSSHHHHHH

SEQ ID NO: 32 - protein, 1866 aa, Eubacterium rectale
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSDTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTCSGGSKRTADGSEFEPKKKRKV

SEQ ID NO: 33 - protein, 2049 aa, unidentified
  source 1..2049: note = Human Foamy Virus
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSNQVGHRKIRPHNIATGDYPPRPQKQYPINPKAKPSIQIVIDDLLKQGVLTPQNSTMNTPVYPVPKPDGRWRMVLDYREVNKTIPLTAAQNQHSAGILATIVRQKYKTTLDLANGFWAHPITPESYWLTAFTWQGKQYCWTRLPQGFLNSPALFTADVVDLLKEIPNVQVYVDDIYLSHDDPKEHVQQLEKVFQILLQAGYVVSLKKSEIGQKTVEFLGFNITKEGRGLTDTFKTKLLNITPPKDLKQLQSILGLLNFARNFIPNFAELVQPLYNLIASAKGKYIEWSEENTKQLNMVIEALNTASNLEERLPEQRLVIKVNTSPSAGYVRYYNETGKKPIMYLNYVFSKAELKFSMLEKLLTTMHKALIKAMDLAMGQEILVYSPIVSMTKIQKTPLPERKALPIRWITWMTYLEDPRIQFHYDKTLPELKHIPDVYTSSQSPVKHPSQYEGVFYTDGSAIKSPDPTKSNNAGMGIVHATYKPEYQVLNQWSIPLGNHTAQMAEIAAVEFACKKALKIPGPVLVITDSFYVAESANKELPYWKSNGFVNNKKKPLKHISKWKSIAECLSMKPDITIQHEKGISLQIPVFILKGNALADKLATQGSYVVNSGGSKRTADGSEFEPKKKRKV

SEQ ID NO: 34 - protein, 2036 aa, unidentified
  source 1..2036: note = human endogenous retrovirus K
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSKSRKRRNRVSFLGAATVEPPKPIPLTWKTEKPVWVNQWPLPKQKLEALHLLANEQLEKGHIEPSFSPWNSPVFVIQKKSGKWRMLTDLRAVNAVIQPMGPLQPGLPSPAMIPKDWPLIIIDLKDCFFTIPLAEQDCEKFAFTIPAINNKEPATRFQWKVLPQGMLNSPTICQTFVGRALQPVREKFSDCYIIHYIDDILCAAETKDKLIDCYTFLQAEVANAGLAIASDKIQTSTPFHYLGMQIENRKIKPQKIEIRKDTLKTLNDFQKLLGDINWIRPTLGIPTYAMSNLFSILRGDSDLNSKRMLTPEATKEIKLVEEKIQSAQINRIDPLAPLQLLIFATAHSPTGIIIQNTDLVEWSFLPHSTVKTFTLYLDQIATLIGQTRLRIIKLCGNDPDKIVVPLTKEQVRQAFINSGAWQIGLANFVGIIDNHYPKTKIFQFLKLTTWILPKITRREPLENALTVFTDGSSNGKAAYTGPKERVIKTPYQSAQRAELVAVITVLQDFDQPINIISDSAYVVQATRDVETALIKYSMDDQLNQLFNLLQQTVRKRNFPFYITHIRAHTNLPGPLTKANEQADLLVSSALIKAQELHASGGSKRTADGSEFEPKKKRKV

SEQ ID NO: 35 - protein, 427 aa, Eubacterium rectale; identical to SEQ ID NO: 13

SEQ ID NO: 36 - protein, 597 aa, unidentified
  source 1..597: note = Human endogenous retrovirus K
MKSRKRRNRVSFLGAATVEPPKPIPLTWKTEKPVWVNQWPLPKQKLEALHLLANEQLEKGHIEPSFSPWNSPVFVIQKKSGKWRMLTDLRAVNAVIQPMGPLQPGLPSPAMIPKDWPLIIIDLKDCFFTIPLAEQDCEKFAFTIPAINNKEPATRFQWKVLPQGMLNSPTICQTFVGRALQPVREKFSDCYIIHYIDDILCAAETKDKLIDCYTFLQAEVANAGLAIASDKIQTSTPFHYLGMQIENRKIKPQKIEIRKDTLKTLNDFQKLLGDINWIRPTLGIPTYAMSNLFSILRGDSDLNSKRMLTPEATKEIKLVEEKIQSAQINRIDPLAPLQLLIFATAHSPTGIIIQNTDLVEWSFLPHSTVKTFTLYLDQIATLIGQTRLRIIKLCGNDPDKIVVPLTKEQVRQAFINSGAWQIGLANFVGIIDNHYPKTKIFQFLKLTTWILPKITRREPLENALTVFTDGSSNGKAAYTGPKERVIKTPYQSAQRAELVAVITVLQDFDQPINIISDSAYVVQATRDVETALIKYSMDDQLNQLFNLLQQTVRKRNFPFYITHIRAHTNLPGPLTKANEQADLLVSSALIKAQELHA

SEQ ID NO: 37 - protein, 420 aa, Geobacillus stearothermophilus; identical to SEQ ID NO: 1

SEQ ID NO: 38 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 39 - protein, 654 aa, synthetic construct
TWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 40 - protein, 496 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCL

SEQ ID NO: 41 - protein, 473 aa, synthetic construct
TWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCL

SEQ ID NO: 42 - protein, 2109 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSSGGSSGSETPGTSESATPESSGGSSGGSEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 43 - protein, 2109 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSSGGSSGSETPGTSESATPESSGGSSGGSSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 44 - RNA, 123 nt, synthetic construct
  source 1..123: mol_type = other RNA
nnnnnnnnnnnnnnnnnnnngttttagagctagaaatagcaagttaaaataaggctagtccgttatcaacttgaaaaagtggcaccgagtcggtgcnnnnnnnnnnnnnnnnnnnnttttttt

SEQ ID NO: 45 - RNA, 104 nt, synthetic construct
  source 1..104: mol_type = other RNA
nnnnnnnnnnnnnnnnnnnngttttagtactctgtaatgaaaattacagaatctactaaaacaaggcaaaatgccgtgtttatctcgtcaacttgttggcgaga

SEQ ID NO: 46 - RNA, 103 nt, synthetic construct
  source 1..103: mol_type = other RNA
nnnnnnnnnnnnnnnnnnnngttttagagctagaaatagcaagttaaaataaggctagtccgttatcaacttgaaaaagtggcaccgagtcggtgcttttttt

SEQ ID NO: 47 - RNA, 104 nt, synthetic construct; identical to SEQ ID NO: 45
  source 1..104: mol_type = other RNA

SEQ ID NO: 48 - RNA, 2154 nt, synthetic construct
  source 1..2154: mol_type = other RNA
atgaaacggacagccgacggaagcgagttcgagtcaccaaagaagaagcggaaagtcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtctaa

SEQ ID NO: 49 - protein, 717 aa, synthetic construct
MKRTADGSEFESPKKKRKVTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 50 - RNA, 1416 nt, synthetic construct
  source 1..1416: mol_type = other RNA
atgaaacggacagccgacggaagcgagttcgagtcaccaaagaagaagcggaaagtcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccatctggcggctcaaaaagaaccgccgacggcagcgaattcgagcccaagaagaagaggaaagtctaa

SEQ ID NO: 51 - protein, 471 aa, synthetic construct
MKRTADGSEFESPKKKRKVTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPSGGSKRTADGSEFEPKKKRKV

SEQ ID NO: 52 - RNA, 2085 nt, synthetic construct
  source 1..2085: mol_type = other RNA
atgaaacggacagccgacggaagcgagttcgagtcaccaaagaagaagcggaaagtcacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtctaa

SEQ ID NO: 53 - protein, 694 aa, synthetic construct
MKRTADGSEFESPKKKRKVTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 54 - RNA, 1533 nt, synthetic construct
  source 1..1533: mol_type = other RNA
atgaaacggacagccgacggaagcgagttcgagtcaccaaagaagaagcggaaagtcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtctctggcggctcaaaaagaaccgccgacggcagcgaattcgagcccaagaagaagaggaaagtctaa

SEQ ID NO: 55 - protein, 510 aa, synthetic construct
MKRTADGSEFESPKKKRKVTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVSGGSKRTADGSEFEPKKKRKV

SEQ ID NO: 56 - RNA, 1206 nt, synthetic construct
  source 1..1206: mol_type = other RNA
atgaaacggacagccgacggaagcgagttcgagtcaccaaagaagaagcggaaagtcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagattctggcggctcaaaaagaaccgccgacggcagcgaattcgagcccaagaagaagaggaaagtctaa

SEQ ID NO: 57 - protein, 401 aa, synthetic construct
MKRTADGSEFESPKKKRKVTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDSGGSKRTADGSEFEPKKKRKV

SEQ ID NO: 58 - RNA, 1611 nt, synthetic construct
  source 1..1611: mol_type = other RNA
atgaaacggacagccgacggaagcgagttcgagtcaccaaagaagaagcggaaagtcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgcctttctggcggctcaaaaagaaccgccgacggcagcgaattcgagcccaagaagaagaggaaagtctaa

SEQ ID NO: 59 - protein, 536 aa, synthetic construct
MKRTADGSEFESPKKKRKVTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLSGGSKRTADGSEFEPKKKRKV

SEQ ID NO: 60 - RNA, 1542 nt, synthetic construct
  source 1..1542: mol_type = other RNA
atgaaacggacagccgacggaagcgagttcgagtcaccaaagaagaagcggaaagtcacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgcctttctggcggctcaaaaagaaccgccgacggcagcgaattcgagcccaagaagaagaggaaagtctaa

SEQ ID NO: 61 - protein, 513 aa, synthetic construct
MKRTADGSEFESPKKKRKVTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLSGGSKRTADGSEFEPKKKRKV

SEQ ID NO: 62 - RNA, 2394 nt, synthetic construct
  source 1..2394: mol_type = other RNA
atgaaacggacagccgacggaagcgagttcgagtcaccaaagaagaagcggaaagtcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgcctttctggcggctcaaaaagaaccgccgacggcagcgaattcgagcccaagaagaagaggaaagtcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctatggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 63 - protein, 797 aa, synthetic construct
MKRTADGSEFESPKKKRKVTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLSGGSKRTADGSEFEPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 64 - RNA, 5769 nt, synthetic construct
  source 1..5769: mol_type = other RNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacgctactaacttcagcctgctgaagcaggctggagacgtggaggagaaccctggacctaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgcctttctggcggctcaaaaagaaccgccgacggcagcgaattcgagcccaagaagaagaggaaagtctaa

SEQ ID NO: 65 - protein, 1922 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDATNFSLLKQAGDVEENPGPTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLSGGSKRTADGSEFEPKKKRKV

SEQ ID NO: 66 - RNA, 1950 nt, synthetic construct
  source 1..1950: mol_type = other RNA
atgaaacggacagccgacggaagcgagttcgagtcaccaaagaagaagcggaaagtcaaccaggtgggccacagaaagatccgccctcacaacatcgccaccggagattacccccccagacctcagaaacagtatcctattaaccccaaggccaagcccagcatccagatcgttatcgacgacctgcttaaacagggcgtgctgacccctcagaacagcaccatgaacacccctgtatatcctgtgcctaagcctgatggcaggtggcggatggtcctggactacagagaggtgaacaagactattcccctgaccgcagcccagaaccagcacagcgccggcatcctggccacaatcgtgcggcagaagtacaagacaaccctggatctggctaatggcttctgggcccaccccatcacaccagaaagctactggctgacagcttttacctggcagggcaagcagtactgctggaccagactgccccagggcttcctgaattctcctgccctgttcaccgctgatgtggtggacctgctgaaagaaatccccaatgtgcaggtgtacgtggatgacatctacctgagccacgacgaccctaaagagcacgtgcagcagctggaaaaggtgtttcagatcctgctgcaggccggctacgtggtgagcctgaagaaaagcgagataggacagaagaccgtggaattcctgggatttaacatcacaaaagagggccggggcctgacagacaccttcaagaccaagctgctgaacatcactccccccaaggacctgaaacaactgcaatctattctgggcctgctgaatttcgccagaaacttcatccctaacttcgccgagctggtgcaacctctttataacctgatcgcctccgccaagggaaagtacatcgagtggagcgaggaaaacacaaagcagctgaacatggtgatcgaggccctgaacaccgcttctaatctggaagagcggctgccagagcagagactggtgatcaaggtgaacaccagccccagcgctggctacgtgcggtactacaacgagacaggcaagaaacctatcatgtacctgaactacgtgttcagcaaggctgaactcaagttcagcatgctggaaaaactgctgaccaccatgcacaaggccctcatcaaggccatggacctggctatgggacaggagatcctggtgtacagcccaatcgtgtccatgaccaagatccaaaaaacacctctgcccgaaagaaaggctctgcctatcagatggatcacctggatgacctacctggaagatcctagaatccagttccactacgacaagaccctgcctgagctgaaacatatcccagacgtgtacacctctagccagagccctgtcaagcatcctagccagtacgagggcgttttctacacagacggcagcgccatcaagagccctgatcctacaaagtccaacaacgctggcatgggcatcgtgcacgccacatacaagcccgagtaccaggtgctgaatcagtggtccatccctctgggcaaccacaccgcccaaatggccgaaatcgccgccgtggaattcgcctgcaagaaggcgctgaagatcccaggccctgtgctggtcattacagatagcttctacgtggccgagagcgccaacaaggagctgccctactggaagtctaacggctttgtgaacaacaagaagaagcctctgaagcacatctccaagtggaaatctatcgccgagtgtctgtctatgaagcctgacatcaccatccagcacgagaagggcatcagcctgcagatccctgtgttcatcctgaagggcaacgccctggccgacaagctggccacccagggcagctatgtggtcaattctggcggctcaaaaagaaccgccgacggcagcgaattcgagcccaagaagaagaggaaagtctaa

SEQ ID NO: 67 - protein, 649 aa, synthetic construct
MKRTADGSEFESPKKKRKVNQVGHRKIRPHNIATGDYPPRPQKQYPINPKAKPSIQIVIDDLLKQGVLTPQNSTMNTPVYPVPKPDGRWRMVLDYREVNKTIPLTAAQNQHSAGILATIVRQKYKTTLDLANGFWAHPITPESYWLTAFTWQGKQYCWTRLPQGFLNSPALFTADVVDLLKEIPNVQVYVDDIYLSHDDPKEHVQQLEKVFQILLQAGYVVSLKKSEIGQKTVEFLGFNITKEGRGLTDTFKTKLLNITPPKDLKQLQSILGLLNFARNFIPNFAELVQPLYNLIASAKGKYIEWSEENTKQLNMVIEALNTASNLEERLPEQRLVIKVNTSPSAGYVRYYNETGKKPIMYLNYVFSKAELKFSMLEKLLTTMHKALIKAMDLAMGQEILVYSPIVSMTKIQKTPLPERKALPIRWITWMTYLEDPRIQFHYDKTLPELKHIPDVYTSSQSPVKHPSQYEGVFYTDGSAIKSPDPTKSNNAGMGIVHATYKPEYQVLNQWSIPLGNHTAQMAEIAAVEFACKKALKIPGPVLVITDSFYVAESANKELPYWKSNGFVNNKKKPLKHISKWKSIAECLSMKPDITIQHEKGISLQIPVFILKGNALADKLATQGSYVVNSGGSKRTADGSEFEPKKKRKV

SEQ ID NO: 68 - RNA, 1911 nt, synthetic construct
  source 1..1911: mol_type = other RNA
atgaaacggacagccgacggaagcgagttcgagtcaccaaagaagaagcggaaagtcaaaagcagaaaacggagaaatagagtgtccttcctgggcgctgccacagtggaaccacctaagcccatccctctgacatggaaaacagagaagcctgtgtgggtcaaccagtggcctctgcctaagcagaagctggaggctctccacctgctggccaacgagcagcttgagaagggccacatcgagcccagctttagcccttggaacagccctgtgttcgtgatccagaagaagagcggcaagtggcggatgctgacagatctgagagctgtgaacgccgtgatccaacccatgggccccctgcagccaggcctgccttcccctgctatgatccctaaagattggcctctgatcatcatcgacctgaaagactgcttcttcacaatcccactcgccgagcaggattgcgagaagttcgccttcaccatccccgccatcaacaacaaggagcctgccaccagattccagtggaaggtgctgcctcagggcatgctgaattctccaacaatctgccagaccttcgtgggcagagctctgcagcctgttagagaaaaattcagcgactgctacatcattcactacatcgatgacatcctgtgcgccgctgaaaccaaggataagttgatcgactgttacaccttcctgcaagccgaggtggccaatgccggactggctatcgcctctgataagatccagaccagcacacctttccactacctgggcatgcagatcgagaaccggaagatcaagccacagaaaatcgagatcagaaaggacaccctgaagaccctgaacgacttccagaaactcctgggggatatcaactggatcagacctaccctgggaatccctacgtacgccatgagcaacctgttcagcatcctgaggggcgacagcgacctgaacagcaagagaatgctgacccctgaggccacaaaagagatcaagctggtggaagagaagatccagtctgctcaaatcaacagaatcgatcccctggcccctcttcagttgctgattttcgccactgcccatagccccaccggcattatcatccagaacaccgacctggtggaatggtcttttctgccccacagcaccgtgaagacatttacactgtacctggaccagatcgccaccctgatcggccaaacaagactgcggatcatcaagctgtgtggcaacgaccccgacaagatcgtggtgcctctgaccaaggaacaggtgcggcaggcttttattaactccggcgcctggcagatcggactggccaacttcgttggcatcatcgacaatcactatcctaagaccaagatcttccaatttctgaagctgaccacctggattctgcctaagattacaagacgggaacccctggagaacgccctgaccgtgttcaccgacggatcttccaacggcaaagccgcctacaccggccctaaggaaagagtgattaagacaccataccagagcgcccagagagccgaactggtcgccgtgatcaccgtgctgcaggacttcgaccagcctatcaatatcatcagcgacagtgcctatgtggtgcaggccacccgggacgtggaaaccgccctgatcaagtacagcatggacgatcagctcaaccagctgtttaacctgctgcagcagaccgtgcggaagagaaacttccccttctacatcacccacatccgcgcccacaccaacctgcccggccctctgacaaaggccaatgagcaggctgatctgctggtgtctagcgccctgattaaggcccaggagctgcacgcctctggcggctcaaaaagaaccgccgacggcagcgaattcgagcccaagaagaagaggaaagtctaa

SEQ ID NO: 69 - protein, 636 aa, synthetic construct
MKRTADGSEFESPKKKRKVKSRKRRNRVSFLGAATVEPPKPIPLTWKTEKPVWVNQWPLPKQKLEALHLLANEQLEKGHIEPSFSPWNSPVFVIQKKSGKWRMLTDLRAVNAVIQPMGPLQPGLPSPAMIPKDWPLIIIDLKDCFFTIPLAEQDCEKFAFTIPAINNKEPATRFQWKVLPQGMLNSPTICQTFVGRALQPVREKFSDCYIIHYIDDILCAAETKDKLIDCYTFLQAEVANAGLAIASDKIQTSTPFHYLGMQIENRKIKPQKIEIRKDTLKTLNDFQKLLGDINWIRPTLGIPTYAMSNLFSILRGDSDLNSKRMLTPEATKEIKLVEEKIQSAQINRIDPLAPLQLLIFATAHSPTGIIIQNTDLVEWSFLPHSTVKTFTLYLDQIATLIGQTRLRIIKLCGNDPDKIVVPLTKEQVRQAFINSGAWQIGLANFVGIIDNHYPKTKIFQFLKLTTWILPKITRREPLENALTVFTDGSSNGKAAYTGPKERVIKTPYQSAQRAELVAVITVLQDFDQPINIISDSAYVVQATRDVETALIKYSMDDQLNQLFNLLQQTVRKRNFPFYITHIRAHTNLPGPLTKANEQADLLVSSALIKAQELHASGGSKRTADGSEFEPKKKRKV

SEQ ID NO: 70 - RNA, 1917 nt, synthetic construct
  source 1..1917: mol_type = other RNA
atgaaacggacagccgacggaagcgagttcgagtcaccaaagaagaagcggaaagtcaagcccacaatggccatcctggaaagaatctctaagaacagccaggagaacatcgacgaggtgttcaccaggctgtaccggtacctgctgagacctgacatctactacgtggcctaccagaacctgtacagcaacaaaggcgcttctaccaagggcatcctcgacgacacagccgacggatttagcgaggaaaaaatcaagaagatcatccagagcctgaaggacggcacctactatcctcaacctgttagaagaatgtatatcgccaagaaaaacagcaagaaaatgcggcctctcggcattccaacattcacagataaactgatccaggaggccgtgcggatcatcctggagtccatctacgagcctgtgttcgaggacgtgagccacggctttagacctcaacgttcttgtcacaccgccctgaaaaccatcaagagagagttcggcggagctcggtggttcgtggaaggcgacatcaagggttgttttgacaacatcgaccacgtgacactgatcggcctgatcaacctgaagattaaggatatgaagatgagccaactgatctacaagtttctgaaggccggctacctggaaaactggcagtatcacaaaacgtacagcggcacacctcagggcggcatcctgagccctctgctggctaatatctacctgcacgagctggacaagttcgtgctgcagctgaaaatgaaattcgatagagaaagccccgagagaatcacccctgagtacagagagctccacaacgagatcaagagaatcagccaccggcttaagaagctggaaggcgaggaaaaagccaaggtgctgctggaataccaggagaagcggaagcggctgcctactctgccctgcaccagccagaccaacaaggtgctgaagtacgtgcggtacgctgatgacttcatcatttctgtgaagggctccaaagaggattgccagtggatcaaggaacagctgaaattgtttatccataacaagctgaagatggagctgtccgaagaaaagaccctgatcacacacagctcccagccagccagattcctgggctacgacatcagagtgcggaggagcggcaccatcaagagaagcggcaaggtgaaaaaacgcaccctgaacggcagcgtcgagctgctgatacccctacaggacaagatcagacagttcatcttcgacaagaaaatcgccatccaaaagaaggacagcagctggttccccgtccatagaaagtacctgattagaagcaccgatctggaaatcatcacaatctacaactctgagctgagaggaatctgcaactactacggcctggctagcaacttcaaccagctgaattacttcgcctacctgatggaatactcctgcctgaagaccatcgccagcaagcacaagggtaccctgtcgaagaccatcagcatgttcaaggatggatctggctcttggggcatcccctacgagatcaagcagggaaagcagagaagatacttcgccaatttcagcgagtgcaagagcccttatcagtttaccgacgagatcagccaggcccctgtgctgtacggatatgcccggaacaccctcgagaatagactgaaagccaagtgctgcgagctgtgtggcacatctgatgaaaataccagctacgagatccaccacgtgaacaaggtgaagaacctgaagggcaaggaaaagtgggagatggccatgatcgccaagcagagaaagacactggtggtgtgcttccactgtcaccgccacgtaatccataagcacaagtctggcggctcaaaaagaaccgccgacggcagcgaattcgagcccaagaagaagaggaaagtctaa

SEQ ID NO: 71 - protein, 638 aa, synthetic construct
MKRTADGSEFESPKKKRKVKPTMAILERISKNSQENIDEVFTRLYRYLLRPDIYYVAYQNLYSNKGASTKGILDDTADGFSEEKIKKIIQSLKDGTYYPQPVRRMYIAKKNSKKMRPLGIPTFTDKLIQEAVRIILESIYEPVFEDVSHGFRPQRSCHTALKTIKREFGGARWFVEGDIKGCFDNIDHVTLIGLINLKIKDMKMSQLIYKFLKAGYLENWQYHKTYSGTPQGGILSPLLANIYLHELDKFVLQLKMKFDRESPERITPEYRELHNEIKRISHRLKKLEGEEKAKVLLEYQEKRKRLPTLPCTSQTNKVLKYVRYADDFIISVKGSKEDCQWIKEQLKLFIHNKLKMELSEEKTLITHSSQPARFLGYDIRVRRSGTIKRSGKVKKRTLNGSVELLIPLQDKIRQFIFDKKIAIQKKDSSWFPVHRKYLIRSTDLEIITIYNSELRGICNYYGLASNFNQLNYFAYLMEYSCLKTIASKHKGTLSKTISMFKDGSGSWGIPYEIKQGKQRRYFANFSECKSPYQFTDEISQAPVLYGYARNTLENRLKAKCCELCGTSDENTSYEIHHVNKVKNLKGKEKWEMAMIAKQRKTLVVCFHCHRHVIHKHKSGGSKRTADGSEFEPKKKRKV

SEQ ID NO: 72 - RNA, 1806 nt, synthetic construct
  source 1..1806: mol_type = other RNA
atgaaacggacagccgacggaagcgagttcgagtcaccaaagaagaagcggaaagtcgaaacaaggcagatggccgtggaacagaccaccggcgccgtcaccaaccagacagagacaagctggcactctatcgactgggccaaagccaaccgagaggtgaaaagactgcaggttagaatcgccaaggccgtgaaagagggcagatggggaaaagtgaaggccctccagtggctcctgacccacagcttctacggcaaggccctggccgtgaagcgggtgacagataatagcggctctaagacacccggcgtggacggaatcacctggtccacccaggaacagaaagctcaggccatcaagtctctgagaagacggggctacaagcctcagcctctgcgaagagtgtacatcccaaaggccaatggcaagcaaagacctctgggcatccctaccatgaaagatagagccatgcaggccctgtatgccctggccctggaacctgtggccgagacgaccgccgatcggaacagctacggctttagaagaggaagatgcatcgctgacgcagctacacagtgccacatcacactggcaaagaccgatcgtgctcagtacgtgctggatgccgatatcgccggatgttttgacaatattagccacgagtggctgctggctaacatccccctggacaagcggatcctgagaaagtggctgaagtccggctttgtgtggaagcagcagctgttccccatccacgccggcacacctcaaggcggggtgatcagccctatgctggcgaacatgaccctggacggcatggaagagctgctgaacaagttccctagagcccacaaggtgaaactgatccggtacgccgacgatttcgtggtgaccggcgagaccaaggaagtgctgtacatagccggagccgtgatccaggctttcctgaaggaaagaggcctgaccctgagcaaggaaaagaccaagattgtccatatcgaggaagggttcgacttcctgggctggaacatccggaaatacgacggcaagctgctgatcaaaccagccaagaagaacgtgaaggcctttctcaagaagatccgggacaccctgagagagctgagaacagcccctcaggagatcgtgatcgatacccttaatccaatcattagaggctggactaactatcacaagaaccaggccagcaaggagacattcgtaggcgtcgaccacctgatctggcagaagctgtggcggtgggccagacggcggcaccccagcaagagcgtgcggtgggtgaagtccaagtacttcatccaaatcggcaaccggaagtggatgttcggcatctggaccaaggacaagaacggcgacccctgggccaaacatctgatcaaggcttctgagatcagaatccagagacgcggcaagatcaaggccgacgccaaccccttcctgcctgagtgggctgagtacttcgagcagcggaagaagctgaaggaagcccctgcccaatacagaagaaccagacgggaactgtggaagaaacagggcggaatctgccctgtgtgtggcggcgagattgagcaggacatgctgacagagatccaccacatcctgcctaagcacaagggcggcaccgacgacctggacaacctggtgctgatccacaccaactgccacaaacaggtgcacaacagagatggacagcacagcagattcctgctgaaggaaggcctgtctggcggctcaaaaagaaccgccgacggcagcgaattcgagcccaagaagaagaggaaagtctaa

SEQ ID NO: 73 - protein, 601 aa, synthetic construct
MKRTADGSEFESPKKKRKVETRQMAVEQTTGAVTNQTETSWHSIDWAKANREVKRLQVRIAKAVKEGRWGKVKALQWLLTHSFYGKALAVKRVTDNSGSKTPGVDGITWSTQEQKAQAIKSLRRRGYKPQPLRRVYIPKANGKQRPLGIPTMKDRAMQALYALALEPVAETTADRNSYGFRRGRCIADAATQCHITLAKTDRAQYVLDADIAGCFDNISHEWLLANIPLDKRILRKWLKSGFVWKQQLFPIHAGTPQGGVISPMLANMTLDGMEELLNKFPRAHKVKLIRYADDFVVTGETKEVLYIAGAVIQAFLKERGLTLSKEKTKIVHIEEGFDFLGWNIRKYDGKLLIKPAKKNVKAFLKKIRDTLRELRTAPQEIVIDTLNPIIRGWTNYHKNQASKETFVGVDHLIWQKLWRWARRRHPSKSVRWVKSKYFIQIGNRKWMFGIWTKDKNGDPWAKHLIKASEIRIQRRGKIKADANPFLPEWAEYFEQRKKLKEAPAQYRRTRRELWKKQGGICPVCGGEIEQDMLTEIHHILPKHKGGTDDLDNLVLIHTNCHKQVHNRDGQHSRFLLKEGLSGGSKRTADGSEFEPKKKRKV

SEQ ID NO: 74 - RNA, 1365 nt, synthetic construct
  source 1..1365: mol_type = other RNA
atgaaacggacagccgacggaagcgagttcgagtcaccaaagaagaagcggaaagtcgatgagacaaagccctacgagatttctaaggacatcgtgcaggaggcctttcagagagtgaaagccaacaagggcgccgccggcgtggacgatgaaaacatcgccgcttttgagagcgacctgaccaacaacctgtacaagatctggaacagaatgagcagcggctgctacttcccacctagcgtgaaggccatcgaaatccctaagaaatctggcggcaccagaatcctgggaatccccacagtgctggacagagtggcccagatggtgaccaaaatctacctggaaccccagctggaacctctgttccaccccgacagctacggctatagacccggcaagtccgccgccgatgccctggctgctacacggaagcggtgctggcggtacaattggctgctggaattcgatatcaagggcctctttgacaacatcaatcacgacctgctgatgaaacaggtgagcatgcataccgacaagccttggatcatcctgtacatccagcgctggctgaaggcccctttccaaatggccgacggcacagtgaatgagcggaccaagggcacccctcagggcggagtggtgtccccactgctggctaatctgttcctgcactacgccttcgaccagtggatggacagccaccacagatacaaccccttcgagcggtatgccgacgacagcgtgatccactgcagatctagagaggaagccgagagactgtggatcgagctggataagagactgagcgagttcggcctggaactgcacccaagcaagacaagaatcgtgtactgtaaagacgatgatagacagggagattaccctgagacaaaattcgacttcctgggctacaccttccggcctagacggagcaagaacaagtacggaaaacatttcatcaacttcacccctgccgtctccaacaccgccaagaagagcatgcagcaggagatccacgattggcggatgcacctgaagcctgacaagaccctggaggacctgtctcacatgttcaaccctatcctgagaggctgggtcaactactacggcctgttctacaagtctgagctgtactgcgtgcttaagcacatgaacagagttctgacccggtgggctcaaagaaaatataagaagctggccggccacaagcggagagccagatactggctgggcaagatcgccagaagggaccccaagctgtttgtgcactggcagatgggcattttccctgaagctggatctggcggctcaaaaagaaccgccgacggcagcgaattcgagcccaagaagaagaggaaagtctaa

SEQ ID NO: 75 - protein, 454 aa, synthetic construct
MKRTADGSEFESPKKKRKVDETKPYEISKDIVQEAFQRVKANKGAAGVDDENIAAFESDLTNNLYKIWNRMSSGCYFPPSVKAIEIPKKSGGTRILGIPTVLDRVAQMVTKIYLEPQLEPLFHPDSYGYRPGKSAADALAATRKRCWRYNWLLEFDIKGLFDNINHDLLMKQVSMHTDKPWIILYIQRWLKAPFQMADGTVNERTKGTPQGGVVSPLLANLFLHYAFDQWMDSHHRYNPFERYADDSVIHCRSREEAERLWIELDKRLSEFGLELHPSKTRIVYCKDDDRQGDYPETKFDFLGYTFRPRRSKNKYGKHFINFTPAVSNTAKKSMQQEIHDWRMHLKPDKTLEDLSHMFNPILRGWVNYYGLFYKSELYCVLKHMNRVLTRWAQRKYKKLAGHKRRARYWLGKIARRDPKLFVHWQMGIFPEAGSGGSKRTADGSEFEPKKKRKV

SEQ ID NO: 76 - RNA, 1380 nt, synthetic construct
  source 1..1380: mol_type = other RNA
atgaaacggacagccgacggaagcgagttcgagtcaccaaagaagaagcggaaagtcgccctgctggagcggatcctggccagagacaatctgatcaccgccctgaaaagggttgaggccaaccagggcgcccctggcatcgacggcgtgtctacagaccagctgagagattacatcagagctcattggagcaccatccacgcccaactcctcgctggcacctacagacccgcccctgtgcggagagtggaaatccccaagcctggaggaggcaccagacagctgggaatccctacagtggtggatagactgatccagcaggccatcctgcaggagcttacaccaatctttgatcctgacttcagcagcagctctttcggcttccggcctggcagaaacgcccacgacgccgttcggcaggcccagggctacatccaagagggctaccggtacgtggtggacatggacctggagaaattcttcgacagagtgaaccacgatatcctgatgtccagagtcgccagaaaggtcaaggacaagcgtgtgctgaaactgatccgggcctacctgcaagctggagtgatgatcgagggcgtgaaagtgcagacagaggaaggaacccctcagggcggccctttgtctcctctgctcgctaacatcctgctggacgacctggataaggagctggaaaagagaggcctgaagttctgcagatacgccgatgactgtaatatctacgtgaagtccctgcgggccggccagagagtgaagcagagcatccagaggttcctggaaaagacactgaagctgaaggtgaacgaggaaaagagcgccgtggacagaccctggaagcgggccttcctgggatttagcttcacccccgaaagaaaggccagaatccgcctggctcccagaagcatccagcggctgaaacagcggattcggcagctgactaaccccaactggtccatcagcatgcctgagagaattcacagagtgaatcagtacgtgatgggctggatcggctattttagactggtggagacacctagcgtgctgcagaccatcgagggttggattagacggagactgagactgtgccagtggctgcagtggaagcgcgtgcgaacaagaatcagagagctgcgggccctgggcctgaaggaaaccgccgtgatggaaatcgccaacaccagaaagggcgcctggcggaccaccaagaccccacagctgcaccaggctctgggcaagacctactggaccgctcagggcctgaaaagcctgacacagagatatttcgagctgagacaaggctctggcggctcaaaaagaaccgccgacggcagcgaattcgagcccaagaagaagaggaaagtctaa

SEQ ID NO: 77 - protein, 459 aa, synthetic construct
MKRTADGSEFESPKKKRKVALLERILARDNLITALKRVEANQGAPGIDGVSTDQLRDYIRAHWSTIHAQLLAGTYRPAPVRRVEIPKPGGGTRQLGIPTVVDRLIQQAILQELTPIFDPDFSSSSFGFRPGRNAHDAVRQAQGYIQEGYRYVVDMDLEKFFDRVNHDILMSRVARKVKDKRVLKLIRAYLQAGVMIEGVKVQTEEGTPQGGPLSPLLANILLDDLDKELEKRGLKFCRYADDCNIYVKSLRAGQRVKQSIQRFLEKTLKLKVNEEKSAVDRPWKRAFLGFSFTPERKARIRLAPRSIQRLKQRIRQLTNPNWSISMPERIHRVNQYVMGWIGYFRLVETPSVLQTIEGWIRRRLRLCQWLQWKRVRTRIRELRALGLKETAVMEIANTRKGAWRTTKTPQLHQALGKTYWTAQGLKSLTQRYFELRQGSGGSKRTADGSEFEPKKKRKV

SEQ ID NO: 78 - RNA, 1401 nt, synthetic construct
  source 1..1401: mol_type = other RNA
atgaaacggacagccgacggaagcgagttcgagtcaccaaagaagaagcggaaagtcgacaccagcaatctgatggaacagatcctgagcagcgacaacctgaaccgggcctacctgcaggtggtgagaaataaaggcgctgaaggcgttgatggcatgaagtacaccgagctgaaggagcatctggccaagaacggcgagacaatcaagggccagctgagaaccagaaagtataagcctcagccagctagacgggtggaaatccccaagcccgatggcggagtgcggaacctgggagtgccaacagtcacagaccggttcatccagcaggctatcgcccaagtgctgacccctatctacgaggaacagtttcacgaccactcttacggcttccggcccaacagatgcgcccagcaagccatcctgacagccctgaacatcatgaacgatggtaatgactggatcgtggacatcgacctggaaaagtttttcgataccgtgaatcacgataagctgatgacgctgattggcagaaccatcaaggacggcgacgtgatctctattgtgcgcaagtacctcgtgtccggcatcatgatcgatgacgagtacgaagatagcatcgtgggaacacctcagggcggcaacctgtctcctctgctggccaacatcatgctgaacgagctggataaggagatggaaaaaaggggcctgaacttcgtgcggtacgccgacgactgcatcatcatggtcggctccgagatgagcgccaacagagtcatgcggaacatcagcagattcatcgaagagaagctgggcctgaaagtgaacatgaccaagtccaaggtggacagacctagcggactgaagtacttgggctttggcttctacttcgaccccagagcccaccagttcaaggccaagcctcacgccaagagcgtggctaagttcaaaaagagaatgaaagagctgacctgtagaagctggggcgtgtctaacagctacaaggtggaaaaactgaatcaactgatcagaggctggatcaactacttcaagatcggcagcatgaagaccctgtgtaaagagctggacagcagaatcaggtacagactgcggatgtgcatctggaagcagtggaaaacccctcagaaccaggagaaaaacctggtcaagcttggaattgacagaaataccgccagaagagtggcctatacaggcaagcgaatcgcctacgtgtgcaacaagggcgccgtgaacgtggctatcagcaacaagcggctggccagcttcggcctgatctctatgctggactactacatcgagaagtgcgtgacctgctctggcggctcaaaaagaaccgccgacggcagcgaattcgagcccaagaagaagaggaaagtctaa

SEQ ID NO: 79 - protein, 466 aa, synthetic construct
MKRTADGSEFESPKKKRKVDTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTCSGGSKRTADGSEFEPKKKRKV

SEQ ID NO: 80 - RNA, 1401 nt, synthetic construct
  source 1..1401: mol_type = other RNA
atgaaacggacagccgacggaagcgagttcgagtcaccaaagaagaagcggaaagtcgacaccagcaatctgatggaacagatcctgagcagccggaacctgaaccgggcctacctgcaggtggtgagacggaaaggcgctgaaggcgttgatggcatgaagtacaccgagctgaaggagcatctggccaagaacggcgagacaatcaagggccagctgagaaccagaaagtataagcctcagccagctagacgggtggaaatccccaagccccggggcggagtgcggaacctgggagtgccaacagtcacagaccggttcatccagcaggctatcgcccaagtgctgacccctatctacgaggaacagtttcacgaccactcttacggcttccggcccaagagatgcgcccagcaagccatcctgacagccctgaacatcatgaacgatggtaatgactggatcgtggacatcgacctggaaaagtttttcgataccgtgaatcacgataagctgatgacgctgattggcagaaccatcaaggacggcgacgtgatctctattgtgcgcaagtacctcgtgtccggcatcatgatcgatgacgagtacgaagatagcatcgtgggaacacctcagggcggccggctgtctcctctgctggccaacatcatgctgaacgagctggataaggagatggaaaaaaggggcctgaacttcgtgcggtacgccgacgactgcatcatcatggtcggctccgagatgagcgccaacagagtcatgcggaacatcagcagattcatcgaagagaagctgggcctgaaagtgaacatgaccaagtccaaggtggacagacctagcggactgaagtacttgggctttggcttctacttcgaccccagagcccaccagttcaaggccaagcctcacgccaagagcgtggctaagttcaaaaagagaatgaaagagctgacctgtagaagctggggcgtgtctaacagctacaaggtggaaaaactgaatcaactgatcagaggctggatcaactacttcaagatcggcagcatgaagaccctgtgtaaagagctggacagcagaatcaggtacagactgcggatgtgcatctggaagcagtggaaaacccctcagaaccaggagaaaaacctggtcaagcttggaattgacagaaataccgccagaagagtggcctatacaggcaagcgaatcgcctacgtgtgcaacaagggcgccgtgaacgtggctatcagcaacaagcggctggccagcttcggcctgatctctatgctggactactacatcgagaagtgcgtgacctgctctggcggctcaaaaagaaccgccgacggcagcgaattcgagcccaagaagaagaggaaagtctaa

SEQ ID NO: 81 - protein, 466 aa, synthetic construct
MKRTADGSEFESPKKKRKVDTSNLMEQILSSRNLNRAYLQVVRRKGAEGVDGMKYTELKEHLAKNGETIKGQLRTRKYKPQPARRVEIPKPRGGVRNLGVPTVTDRFIQQAIAQVLTPIYEEQFHDHSYGFRPKRCAQQAILTALNIMNDGNDWIVDIDLEKFFDTVNHDKLMTLIGRTIKDGDVISIVRKYLVSGIMIDDEYEDSIVGTPQGGRLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTCSGGSKRTADGSEFEPKKKRKV

SEQ ID NO: 82 - RNA, 7137 nt, synthetic construct
  source 1..7137: mol_type = other RNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctatggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 83 - protein, 2378 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 84 - RNA, 7137 nt, synthetic construct
  source 1..7137: mol_type = other RNA
atgaaacggacagccgacggaagcgagttcgagtcaccaaagaagaagcggaaagtcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggaggatctagcggaggatcctctggcagcgagacaccaggaacaagcgagtcagcaacaccagagagcagtggcggcagcagcggcggcagcagcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctatggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 85 - protein, 2378 aa, synthetic construct
MKRTADGSEFESPKKKRKVTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSSGGSSGSETPGTSESATPESSGGSSGGSSDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 86 - RNA, 7230 nt, synthetic construct
  source 1..7230: mol_type = other RNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggcggaggatctagcggaggatcctctggaagcgagacaccaggcacaagcgagtccgccacaccagagagctccggcggctcctccggaggatcctctaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctccggaggatctagcggaggctcctctggctctgagacacctggcacaagcgagagcgcaacacctgaaagcagcgggggcagcagcggggggtcatcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctatggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 87 - protein, 2409 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSSGGSSGSETPGTSESATPESSGGSSGGSSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 88 - RNA, 5106 nt, synthetic construct
  source 1..5106: mol_type = other RNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagctctggcggctcaaaaagaaccgccgacggcagcgaattcgagcccaagaagaagaggaaagtcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctatggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 89 - protein, 1701 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSSGGSKRTADGSEFEPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 90 - RNA, 2937 nt, synthetic construct
  source 1..2937: mol_type = other RNA
atgaaacggacagccgacggaagcgagttcgagtcaccaaagaagaagcggaaagtcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctatggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 91 - protein, 978 aa, synthetic construct
MKRTADGSEFESPKKKRKVTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 92 - RNA, 7230 nt, synthetic construct
  source 1..7230: mol_type = other RNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcggaggatctagcggaggatcctctggaagcgagacaccaggcacaagcgagtccgccacaccagagagctccggcggctcctccggaggatcctctaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctccggaggatctagcggaggctcctctggctctgagacacctggcacaagcgagagcgcaacacctgaaagcagcgggggcagcagcggggggtcagagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctatggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 93 - protein, 2409 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSSGGSSGSETPGTSESATPESSGGSSGGSEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 94 - RNA, 6192 nt, synthetic construct
  source 1..6192: mol_type = other RNA
atgaaacggacagccgacggaagcgagttcgagtcaccaaagaagaagcggaaagtcaaacggaactacatcctggggcttgacattgggataaccagcgttggctacggaattattgattatgagacacgcgatgtgattgacgccggggttaggctgttcaaagaggccaacgttgaaaacaacgagggaagacggagtaagcgcggagcaagaagactcaagcgcagacggagacatcggattcagagggtgaaaaagctgctcttcgattacaatctcctgaccgatcatagtgagctgagcggaatcaacccctacgaggcgcgagtgaaagggctttcccagaagctgtccgaagaggagttctccgccgcgttgctgcacctggccaaacggaggggggttcacaatgtaaacgaagtggaggaggacacgggcaatgaacttagtacgaaagaacagatcagtaggaactctaaggctctcgaagagaaatacgtcgctgagttgcagcttgagagactgaaaaaagacggcgaagtacgcggatctattaataggttcaagacttcagattacgtaaaggaagccaagcagctcctgaaagtacagaaagcgtaccatcagctcgatcagagcttcatcgatacctacatagatttgctggagacacggaggacatactacgagggcccaggggaaggatctccttttgggtggaaggacatcaaggaatggtacgagatgcttatgggacattgtacatattttccggaggagctcaggagcgtcaagtacgcctacaatgccgacctgtacaatgccctcaatgacctcaataacctcgtgattaccagggacgagaacgagaagctggagtactatgaaaagttccagattatcgagaatgtgtttaagcagaagaagaagccgacacttaagcagattgcaaaggaaatcctcgtgaatgaggaagatatcaagggatacagagtgacaagtacaggcaagcccgagttcacaaatctgaaggtgtaccacgatattaaggacataaccgcacgaaaggagataatcgaaaacgctgagctcctcgatcagatcgcaaaaattcttaccatctaccagtctagtgaggacattcaggaggaactgactaatctgaacagtgagctcacccaagaggaaattgagcagatttcaaacctgaaaggctacaccgggacgcacaatctgagcctcaaagcaatcaacctcattctggatgaactttggcacacaaatgacaaccaaattgccatattcaaccgcctgaaactggtgccaaaaaaagtggatctgtcacagcaaaaggaaatccctacaaccttggttgacgattttattctgtcccccgttgtcaagcggagcttcatccagtcaatcaaggtgatcaatgccatcattaaaaaatacggattgccaaacgatataattatcgagcttgcacgagagaagaactcaaaggacgcccagaagatgattaacgaaatgcagaagcgcaaccgccagacaaacgaacgcatagaggaaattataagaacaaccggcaaagagaatgccaagtatctgatcgagaaaatcaagctgcacgacatgcaagaaggcaagtgcctgtactctctggaagctatcccactcgaagatctgctgaataatccattcaattacgaggtggaccacatcatccctagatccgtaagctttgacaattccttcaataacaaagttctggttaaacaggaggaagcctctaaaaaagggaaccggaccccgttccagtacctgagctccagtgacagcaagattagctacgagacttttaagaaacatattctgaatctggccaaaggcaaaggcaggatcagcaagaccaagaaggagtacctcctcgaagaacgcgacattaacagatttagtgtgcagaaagatttcatcaaccgaaaccttgtcgatactcggtacgccacgagaggcctgatgaatctcctcaggagctacttccgcgtcaataatctggacgttaaagtcaagagcataaatgggggattcaccagctttctgaggagaaagtggaagtttaagaaggaacgaaacaaaggatacaagcaccatgctgaggatgctttgatcatcgctaacgcggactttatctttaaggaatggaaaaagctggataaggcaaagaaagtgatggaaaaccagatgttcgaggagaagcaggcagagtcaatgcctgagatcgagacagagcaggaatacaaggaaattttcatcacccctcatcagattaaacacataaaggacttcaaagactataaatactctcatagggtggacaaaaaacccaatcgcaagctcattaatgacaccctgtactcaacacggaaggatgataaaggtaataccttgattgtgaataatcttaatggattgtatgacaaagataacgacaagctcaagaagctgatcaacaagtctccagagaagctccttatgtatcaccacgacccacagacttatcagaaattgaaactgatcatggagcaatacggggatgagaagaacccactctacaaatattatgaggaaacaggtaattacctgaccaagtactccaagaaggataacggaccagtgatcaaaaagataaagtactatggcaacaaacttaatgcgcatttggacataactgacgattaccccaattctcgaaacaaggttgtgaagctctccctgaagccttatagatttgacgtgtacctggataatggggtttataaattcgtcaccgtgaaaaatctggacgtgatcaaaaaggagaactattatgaagtaaactcaaagtgctatgaggaggcgaagaagctgaagaagatctccaatcaggccgagttcatcgcttccttctataagaacgatctcatcaagatcaatggagagctttatcgcgtcattggtgtgaacaatgacttgctgaacaggatcgaagtcaatatgatagacattacctaccgggagtatctcgaaaacatgaatgataaacggccgcctcacatcatcaagacaatcgcatctaaaactcagtcaataaaaaagtactctaccgatatcctggggaatctctatgaagtgaagtcaaagaagcacccacaaatcattaaaaaaggttctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctatggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 95 - protein, 2063 aa, synthetic construct
MKRTADGSEFESPKKKRKVKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKGSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 96 - RNA, 5649 nt, synthetic construct
  source 1..5649: mol_type = other RNA
atgaaacggacagccgacggaagcgagttcgagtcaccaaagaagaagcggaaagtcaaacggaactacatcctggggcttgacattgggataaccagcgttggctacggaattattgattatgagacacgcgatgtgattgacgccggggttaggctgttcaaagaggccaacgttgaaaacaacgagggaagacggagtaagcgcggagcaagaagactcaagcgcagacggagacatcggattcagagggtgaaaaagctgctcttcgattacaatctcctgaccgatcatagtgagctgagcggaatcaacccctacgaggcgcgagtgaaagggctttcccagaagctgtccgaagaggagttctccgccgcgttgctgcacctggccaaacggaggggggttcacaatgtaaacgaagtggaggaggacacgggcaatgaacttagtacgaaagaacagatcagtaggaactctaaggctctcgaagagaaatacgtcgctgagttgcagcttgagagactgaaaaaagacggcgaagtacgcggatctattaataggttcaagacttcagattacgtaaaggaagccaagcagctcctgaaagtacagaaagcgtaccatcagctcgatcagagcttcatcgatacctacatagatttgctggagacacggaggacatactacgagggcccaggggaaggatctccttttgggtggaaggacatcaaggaatggtacgagatgcttatgggacattgtacatattttccggaggagctcaggagcgtcaagtacgcctacaatgccgacctgtacaatgccctcaatgacctcaataacctcgtgattaccagggacgagaacgagaagctggagtactatgaaaagttccagattatcgagaatgtgtttaagcagaagaagaagccgacacttaagcagattgcaaaggaaatcctcgtgaatgaggaagatatcaagggatacagagtgacaagtacaggcaagcccgagttcacaaatctgaaggtgtaccacgatattaaggacataaccgcacgaaaggagataatcgaaaacgctgagctcctcgatcagatcgcaaaaattcttaccatctaccagtctagtgaggacattcaggaggaactgactaatctgaacagtgagctcacccaagaggaaattgagcagatttcaaacctgaaaggctacaccgggacgcacaatctgagcctcaaagcaatcaacctcattctggatgaactttggcacacaaatgacaaccaaattgccatattcaaccgcctgaaactggtgccaaaaaaagtggatctgtcacagcaaaaggaaatccctacaaccttggttgacgattttattctgtcccccgttgtcaagcggagcttcatccagtcaatcaaggtgatcaatgccatcattaaaaaatacggattgccaaacgatataattatcgagcttgcacgagagaagaactcaaaggacgcccagaagatgattaacgaaatgcagaagcgcaaccgccagacaaacgaacgcatagaggaaattataagaacaaccggcaaagagaatgccaagtatctgatcgagaaaatcaagctgcacgacatgcaagaaggcaagtgcctgtactctctggaagctatcccactcgaagatctgctgaataatccattcaattacgaggtggaccacatcatccctagatccgtaagctttgacaattccttcaataacaaagttctggttaaacaggaggaagcctctaaaaaagggaaccggaccccgttccagtacctgagctccagtgacagcaagattagctacgagacttttaagaaacatattctgaatctggccaaaggcaaaggcaggatcagcaagaccaagaaggagtacctcctcgaagaacgcgacattaacagatttagtgtgcagaaagatttcatcaaccgaaaccttgtcgatactcggtacgccacgagaggcctgatgaatctcctcaggagctacttccgcgtcaataatctggacgttaaagtcaagagcataaatgggggattcaccagctttctgaggagaaagtggaagtttaagaaggaacgaaacaaaggatacaagcaccatgctgaggatgctttgatcatcgctaacgcggactttatctttaaggaatggaaaaagctggataaggcaaagaaagtgatggaaaaccagatgttcgaggagaagcaggcagagtcaatgcctgagatcgagacagagcaggaatacaaggaaattttcatcacccctcatcagattaaacacataaaggacttcaaagactataaatactctcatagggtggacaaaaaacccaatcgcaagctcattaatgacaccctgtactcaacacggaaggatgataaaggtaataccttgattgtgaataatcttaatggattgtatgacaaagataacgacaagctcaagaagctgatcaacaagtctccagagaagctccttatgtatcaccacgacccacagacttatcagaaattgaaactgatcatggagcaatacggggatgagaagaacccactctacaaatattatgaggaaacaggtaattacctgaccaagtactccaagaaggataacggaccagtgatcaaaaagataaagtactatggcaacaaacttaatgcgcatttggacataactgacgattaccccaattctcgaaacaaggttgtgaagctctccctgaagccttatagatttgacgtgtacctggataatggggtttataaattcgtcaccgtgaaaaatctggacgtgatcaaaaaggagaactattatgaagtaaactcaaagtgctatgaggaggcgaagaagctgaagaagatctccaatcaggccgagttcatcgcttccttctataagaacgatctcatcaagatcaatggagagctttatcgcgtcattggtgtgaacaatgacttgctgaacaggatcgaagtcaatatgatagacattacctaccgggagtatctcgaaaacatgaatgataaacggccgcctcacatcatcaagacaatcgcatctaaaactcagtcaataaaaaagtactctaccgatatcctggggaatctctatgaagtgaagtcaaagaagcacccacaaatcattaaaaaaggttctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgcctttctggcggctcaaaaagaaccgccgacggcagcgaattcgagcccaagaagaagaggaaagtcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctatggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 97 - protein, 1882 aa, synthetic construct
MKRTADGSEFESPKKKRKVKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKGSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLSGGSKRTADGSEFEPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 98 - RNA, 4161 nt, synthetic construct
  source 1..4161: mol_type = other RNA
atgaaacggacagccgacggaagcgagttcgagtcaccaaagaagaagcggaaagtcaaacggaactacatcctggggcttgacattgggataaccagcgttggctacggaattattgattatgagacacgcgatgtgattgacgccggggttaggctgttcaaagaggccaacgttgaaaacaacgagggaagacggagtaagcgcggagcaagaagactcaagcgcagacggagacatcggattcagagggtgaaaaagctgctcttcgattacaatctcctgaccgatcatagtgagctgagcggaatcaacccctacgaggcgcgagtgaaagggctttcccagaagctgtccgaagaggagttctccgccgcgttgctgcacctggccaaacggaggggggttcacaatgtaaacgaagtggaggaggacacgggcaatgaacttagtacgaaagaacagatcagtaggaactctaaggctctcgaagagaaatacgtcgctgagttgcagcttgagagactgaaaaaagacggcgaagtacgcggatctattaataggttcaagacttcagattacgtaaaggaagccaagcagctcctgaaagtacagaaagcgtaccatcagctcgatcagagcttcatcgatacctacatagatttgctggagacacggaggacatactacgagggcccaggggaaggatctccttttgggtggaaggacatcaaggaatggtacgagatgcttatgggacattgtacatattttccggaggagctcaggagcgtcaagtacgcctacaatgccgacctgtacaatgccctcaatgacctcaataacctcgtgattaccagggacgagaacgagaagctggagtactatgaaaagttccagattatcgagaatgtgtttaagcagaagaagaagccgacacttaagcagattgcaaaggaaatcctcgtgaatgaggaagatatcaagggatacagagtgacaagtacaggcaagcccgagttcacaaatctgaaggtgtaccacgatattaaggacataaccgcacgaaaggagataatcgaaaacgctgagctcctcgatcagatcgcaaaaattcttaccatctaccagtctagtgaggacattcaggaggaactgactaatctgaacagtgagctcacccaagaggaaattgagcagatttcaaacctgaaaggctacaccgggacgcacaatctgagcctcaaagcaatcaacctcattctggatgaactttggcacacaaatgacaaccaaattgccatattcaaccgcctgaaactggtgccaaaaaaagtggatctgtcacagcaaaaggaaatccctacaaccttggttgacgattttattctgtcccccgttgtcaagcggagcttcatccagtcaatcaaggtgatcaatgccatcattaaaaaatacggattgccaaacgatataattatcgagcttgcacgagagaagaactcaaaggacgcccagaagatgattaacgaaatgcagaagcgcaaccgccagacaaacgaacgcatagaggaaattataagaacaaccggcaaagagaatgccaagtatctgatcgagaaaatcaagctgcacgacatgcaagaaggcaagtgcctgtactctctggaagctatcccactcgaagatctgctgaataatccattcaattacgaggtggaccacatcatccctagatccgtaagctttgacaattccttcaataacaaagttctggttaaacaggaggaagcctctaaaaaagggaaccggaccccgttccagtacctgagctccagtgacagcaagattagctacgagacttttaagaaacatattctgaatctggccaaaggcaaaggcaggatcagcaagaccaagaaggagtacctcctcgaagaacgcgacattaacagatttagtgtgcagaaagatttcatcaaccgaaaccttgtcgatactcggtacgccacgagaggcctgatgaatctcctcaggagctacttccgcgtcaataatctggacgttaaagtcaagagcataaatgggggattcaccagctttctgaggagaaagtggaagtttaagaaggaacgaaacaaaggatacaagcaccatgctgaggatgctttgatcatcgctaacgcggactttatctttaaggaatggaaaaagctggataaggcaaagaaagtgatggaaaaccagatgttcgaggagaagcaggcagagtcaatgcctgagatcgagacagagcaggaatacaaggaaattttcatcacccctcatcagattaaacacataaaggacttcaaagactataaatactctcatagggtggacaaaaaacccaatcgcaagctcattaatgacaccctgtactcaacacggaaggatgataaaggtaataccttgattgtgaataatcttaatggattgtatgacaaagataacgacaagctcaagaagctgatcaacaagtctccagagaagctccttatgtatcaccacgacccacagacttatcagaaattgaaactgatcatggagcaatacggggatgagaagaacccactctacaaatattatgaggaaacaggtaattacctgaccaagtactccaagaaggataacggaccagtgatcaaaaagataaagtactatggcaacaaacttaatgcgcatttggacataactgacgattaccccaattctcgaaacaaggttgtgaagctctccctgaagccttatagatttgacgtgtacctggataatggggtttataaattcgtcaccgtgaaaaatctggacgtgatcaaaaaggagaactattatgaagtaaactcaaagtgctatgaggaggcgaagaagctgaagaagatctccaatcaggccgagttcatcgcttccttctataagaacgatctcatcaagatcaatggagagctttatcgcgtcattggtgtgaacaatgacttgctgaacaggatcgaagtcaatatgatagacattacctaccgggagtatctcgaaaacatgaatgataaacggccgcctcacatcatcaagacaatcgcatctaaaactcagtcaataaaaaagtactctaccgatatcctggggaatctctatgaagtgaagtcaaagaagcacccacaaatcattaaaaaaggttctggaggatctagcggaggatcctctggcagcgagacaccaggaacaagcgagtcagcaacaccagagagcagtggcggcagcagcggcggcagcagctctggcggctcaaaaagaaccgccgacggcagcgaattcgagcccaagaagaagaggaaagtcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctatggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 99 - protein, 1386 aa, synthetic construct
MKRTADGSEFESPKKKRKVKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKGSGGSSGGSSGSETPGTSESATPESSGGSSGGSSSGGSKRTADGSEFEPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 100 - RNA, 4161 nt, synthetic construct
  source 1..4161: mol_type = other RNA
atgaaacggacagccgacggaagcgagttcgagtcaccaaagaagaagcggaaagtcaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggctctggaggatctagcggaggatcctctggcagcgagacaccaggaacaagcgagtcagcaacaccagagagcagtggcggcagcagcggcggcagcagctctggcggctcaaaaagaaccgccgacggcagcgaattcgagcccaagaagaagaggaaagtcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctatggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 101 - protein, 1386 aa, synthetic construct
MKRTADGSEFESPKKKRKVKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGSGGSSGGSSGSETPGTSESATPESSGGSSGGSSSGGSKRTADGSEFEPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 102 - RNA, 6594 nt, synthetic construct
  source 1..6594: mol_type = other RNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgcctttctggcggctcaaaaagaaccgccgacggcagcgaattcgagcccaagaagaagaggaaagtcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctatggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 103 - protein, 2197 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLSGGSKRTADGSEFEPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 104 - RNA, 4194 nt, synthetic construct
  source 1..4194: mol_type = other RNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagccccaagaagaagagaaaggtggaggccagctaa

SEQ ID NO: 105 - protein, 1397 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSPKKKRKVEAS

SEQ ID NO: 106 - RNA, 3525 nt, synthetic construct
  source 1..3525: mol_type = other RNA
tgtggactactagtaagcttggatcttgaagaagctgcaggaggtgctggaggggaagtggtccggatccgatcagtgtgagggagtgtaaagctggtttgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcccagactgagcacgtgagttttagagctagaaatagcaagttaaaataaggctagtccgttatcaacttgaaaaagtgggaccgagtcggtcctctgccatcaaagcgtgctcagtctgtttttttggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactttttcccgtcaaccagtatcccggtgcgttttagagctagaaatagcaagttaaaataaggctagtccgttatcaacttgaaaaagtggcaccgagtcggtgctttttttctaactcgaacgctagctgtgcgatcgttttcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcgggacccgacattagcgctacagcttaagcgggccgccaccatgaaacggacagccgacggaagcgagttcgagtcaccaaagaagaagcggaaagtcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgcctttctggcggctcaaaaagaaccgccgacggcagcgaattcgagcccaagaagaagaggaaagtcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctatggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 107 - protein, 1159 aa, synthetic construct
CGLLVSLDLEEAAGGAGGEVVRIRSVGSVKLVGPISHDSFIFAYTIQGCRDNNFDCKHKDISTKYVTKVIISWVVCSFKIMFNGLSYAYRNLKVFRFLGFIYLVERTKHRPRLSTVLELEIASNKASPLSTKSGTESVLCHQSVLSLFFWNSNADVINPLQGIAGPVSLGGNTQRACALAGRWLGTGEWRPAIFACRYVFWEITINVKCLWIWESYKFCMRPLFPVNQYPGAFSKQVKIRLVRYQLEKVAPSRCFFSNSNASCAIVFEWLRCPSVGRAHIAHSPREVGGRGRQLNRCLEKVARGKLGKCRVLAPPFSRGWGRTVYKCSSRRERSFSQRVCRQNTGVVTRDPTLALQLKRAATMKRTADGSEFESPKKKRKVTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLSGGSKRTADGSEFEPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 108 - RNA, 6384 nt, synthetic construct
  source 1..6384: mol_type = other RNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagcgacaccagcaatctgatggaacagatcctgagcagccggaacctgaaccgggcctacctgcaggtggtgagacggaaaggcgctgaaggcgttgatggcatgaagtacaccgagctgaaggagcatctggccaagaacggcgagacaatcaagggccagctgagaaccagaaagtataagcctcagccagctagacgggtggaaatccccaagccccggggcggagtgcggaacctgggagtgccaacagtcacagaccggttcatccagcaggctatcgcccaagtgctgacccctatctacgaggaacagtttcacgaccactcttacggcttccggcccaagagatgcgcccagcaagccatcctgacagccctgaacatcatgaacgatggtaatgactggatcgtggacatcgacctggaaaagtttttcgataccgtgaatcacgataagctgatgacgctgattggcagaaccatcaaggacggcgacgtgatctctattgtgcgcaagtacctcgtgtccggcatcatgatcgatgacgagtacgaagatagcatcgtgggaacacctcagggcggccggctgtctcctctgctggccaacatcatgctgaacgagctggataaggagatggaaaaaaggggcctgaacttcgtgcggtacgccgacgactgcatcatcatggtcggctccgagatgagcgccaacagagtcatgcggaacatcagcagattcatcgaagagaagctgggcctgaaagtgaacatgaccaagtccaaggtggacagacctagcggactgaagtacttgggctttggcttctacttcgaccccagagcccaccagttcaaggccaagcctcacgccaagagcgtggctaagttcaaaaagagaatgaaagagctgacctgtagaagctggggcgtgtctaacagctacaaggtggaaaaactgaatcaactgatcagaggctggatcaactacttcaagatcggcagcatgaagaccctgtgtaaagagctggacagcagaatcaggtacagactgcggatgtgcatctggaagcagtggaaaacccctcagaaccaggagaaaaacctggtcaagcttggaattgacagaaataccgccagaagagtggcctatacaggcaagcgaatcgcctacgtgtgcaacaagggcgccgtgaacgtggctatcagcaacaagcggctggccagcttcggcctgatctctatgctggactactacatcgagaagtgcgtgacctgctctggcggctcaaaaagaaccgccgacggcagcgaattcgagcccaagaagaagaggaaagtcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctatggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 109 - protein, 2127 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSDTSNLMEQILSSRNLNRAYLQVVRRKGAEGVDGMKYTELKEHLAKNGETIKGQLRTRKYKPQPARRVEIPKPRGGVRNLGVPTVTDRFIQQAIAQVLTPIYEEQFHDHSYGFRPKRCAQQAILTALNIMNDGNDWIVDIDLEKFFDTVNHDKLMTLIGRTIKDGDVISIVRKYLVSGIMIDDEYEDSIVGTPQGGRLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTCSGGSKRTADGSEFEPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 110 - RNA, 1401 nt, synthetic construct
  source 1..1401: mol_type = other RNA
atgaaacggacagccgacggaagcgagttcgagtcaccaaagaagaagcggaaagtcgacaccagcaatctgatggaacagatcctgagcagccggaacctgaaccgggcctacctgcaggtggtgagaaataaaggcgctgaaggcgttgatggcatgaagtacaccgagctgaaggagcatctggccaagaacggcgagacaatcaagggccagctgagaaccagaaagtataagcctcagccagctagacgggtggaaatccccaagccccggggcggagtgcggaacctgggagtgccaacagtcacagaccggttcatccagcaggctatcgcccaagtgctgacccctatctacgaggaacagtttcacgaccactcttacggcttccggcccaagagatgcgcccagcaagccatcctgacagccctgaacatcatgaacgatggtaatgactggatcgtggacatcgacctggaaaagtttttcgataccgtgaatcacgataagctgatgacgctgattggcagaaccatcaaggacggcgacgtgatctctattgtgcgcaagtacctcgtgtccggcatcatgatcgatgacgagtacgaagatagcatcgtgggaacacctcagggcggccggctgtctcctctgctggccaacatcatgctgaacgagctggataaggagatggaaaaaaggggcctgaacttcgtgcggtacgccgacgactgcatcatcatggtcggctccgagatgagcgccaacagagtcatgcggaacatcagcagattcatcgaagagaagctgggcctgaaagtgaacatgaccaagtccaaggtggacagacctagcggactgaagtacttgggctttggcttctacttcgaccccagagcccaccagttcaaggccaagcctcacgccaagagcgtggctaagttcaaaaagagaatgaaagagctgacctgtagaagctggggcgtgtctaacagctacaaggtggaaaaactgaatcaactgatcagaggctggatcaactacttcaagatcggcagcatgaagaccctgtgtaaagagctggacagcagaatcaggtacagactgcggatgtgcatctggaagcagtggaaaacccctcagaaccaggagaaaaacctggtcaagcttggaattgacagaaataccgccagaagagtggcctatacaggcaagcgaatcgcctacgtgtgcaacaagggcgccgtgaacgtggctatcagcaacaagcggctggccagcttcggcctgatctctatgctggactactacatcgagaagtgcgtgacctgctctggcggctcaaaaagaaccgccgacggcagcgaattcgagcccaagaagaagaggaaagtctaa

SEQ ID NO: 111 - protein, 466 aa, synthetic construct
MKRTADGSEFESPKKKRKVDTSNLMEQILSSRNLNRAYLQVVRNKGAEGVDGMKYTELKEHLAKNGETIKGQLRTRKYKPQPARRVEIPKPRGGVRNLGVPTVTDRFIQQAIAQVLTPIYEEQFHDHSYGFRPKRCAQQAILTALNIMNDGNDWIVDIDLEKFFDTVNHDKLMTLIGRTIKDGDVISIVRKYLVSGIMIDDEYEDSIVGTPQGGRLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTCSGGSKRTADGSEFEPKKKRKV

SEQ ID NO: 112 - RNA, 2502 nt, synthetic construct
  source 1..2502: mol_type = other RNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtgcctgtcctacgagacagagatcctgacagtggagtatggcctgctgccaatcggcaagatcgtggagaagaggatcgagtgtaccgtgtactctgtggataacaatggcaacatctatacacagcccgtggcacagtggcacgataggggagagcaggaggtgttcgagtattgcctggaggacggcagcctgatcagggcaaccaaggaccacaagttcatgacagtggatggccagatgctgcccatcgacgagattttcgagcgggagctggacctgatgagagtggataacctgcctaattaa

SEQ ID NO: 113 - protein, 833 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 114 - RNA, 4269 nt, synthetic construct
  source 1..4269: mol_type = other RNA
atgatcaagattgctacacggaaatacctgggaaagcagaacgtgtacgacatcggcgtggagcgggatcacaacttcgccctgaagaatggctttatcgccagcaattccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtctaa

SEQ ID NO: 115 - protein, 1422 aa, synthetic construct
MIKIATRKYLGKQNVYDIGVERDHNFALKNGFIASNSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 116 - RNA, 6384 nt, synthetic construct
  source 1..6384: mol_type = other RNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagcgacaccagcaatctgatggaacagatcctgagcagcgacaacctgaaccgggcctacctgcaggtggtgagaaataaaggcgctgaaggcgttgatggcatgaagtacaccgagctgaaggagcatctggccaagaacggcgagacaatcaagggccagctgagaaccagaaagtataagcctcagccagctagacgggtggaaatccccaagcccgatggcggagtgcggaacctgggagtgccaacagtcacagaccggttcatccagcaggctatcgcccaagtgctgacccctatctacgaggaacagtttcacgaccactcttacggcttccggcccaacagatgcgcccagcaagccatcctgacagccctgaacatcatgaacgatggtaatgactggatcgtggacatcgacctggaaaagtttttcgataccgtgaatcacgataagctgatgacgctgattggcagaaccatcaaggacggcgacgtgatctctattgtgcgcaagtacctcgtgtccggcatcatgatcgatgacgagtacgaagatagcatcgtgggaacacctcagggcggcaacctgtctcctctgctggccaacatcatgctgaacgagctggataaggagatggaaaaaaggggcctgaacttcgtgcggtacgccgacgactgcatcatcatggtcggctccgagatgagcgccaacagagtcatgcggaacatcagcagattcatcgaagagaagctgggcctgaaagtgaacatgaccaagtccaaggtggacagacctagcggactgaagtacttgggctttggcttctacttcgaccccagagcccaccagttcaaggccaagcctcacgccaagagcgtggctaagttcaaaaagagaatgaaagagctgacctgtagaagctggggcgtgtctaacagctacaaggtggaaaaactgaatcaactgatcagaggctggatcaactacttcaagatcggcagcatgaagaccctgtgtaaagagctggacagcagaatcaggtacagactgcggatgtgcatctggaagcagtggaaaacccctcagaaccaggagaaaaacctggtcaagcttggaattgacagaaataccgccagaagagtggcctatacaggcaagcgaatcgcctacgtgtgcaacaagggcgccgtgaacgtggctatcagcaacaagcggctggccagcttcggcctgatctctatgctggactactacatcgagaagtgcgtgacctgctctggcggctcaaaaagaaccgccgacggcagcgaattcgagcccaagaagaagaggaaagtcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctatggagtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 117 - protein, 2127 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSDTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTCSGGSKRTADGSEFEPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 118 - RNA, 6384 nt, synthetic construct; identical to SEQ ID NO: 108
  source 1..6384: mol_type = other RNA

SEQ ID NO: 119 - protein, 2127 aa, synthetic construct; identical to SEQ ID NO: 109

SEQ ID NO: 120 - protein, 8 aa, synthetic construct
DYKDDDDK

SEQ ID NO: 121 - protein, 562 aa, unidentified; identical to SEQ ID NO: 3
  source 1..562: note = Thermosynechococcus elongatus

SEQ ID NO: 122 - protein, 552 aa, Streptococcus sp.; identical to SEQ ID NO: 14

SEQ ID NO: 123 - protein, 494 aa, Pseudomonas putida; identical to SEQ ID NO: 10

SEQ ID NO: 124 - protein, 555 aa, Shigella sonnei; identical to SEQ ID NO: 15

SEQ ID NO: 125 - protein, 599 aa, Lactococcus lactis; identical to SEQ ID NO: 2

SEQ ID NO: 126 - protein, 419 aa, Sinorhizobium sp.; identical to SEQ ID NO: 4

SEQ ID NO: 127 - protein, 415 aa, Methanosarcina acetivorans; identical to SEQ ID NO: 5

SEQ ID NO: 128 - protein, 436 aa, Streptococcus agalactiae; identical to SEQ ID NO: 11

SEQ ID NO: 129 - protein, 427 aa, Eubacterium rectale; identical to SEQ ID NO: 13

SEQ ID NO: 130 - protein, 434 aa, Enterobacter cloacae; identical to SEQ ID NO: 6

SEQ ID NO: 131 - protein, 490 aa, Pseudomonas alcaligenes; identical to SEQ ID NO: 9

SEQ ID NO: 132 - protein, 470 aa, Clostridium acetobutylicum; identical to SEQ ID NO: 7

SEQ ID NO: 133 - protein, 420 aa, Geobacillus stearothermophilus; identical to SEQ ID NO: 1

SEQ ID NO: 134 - protein, 418 aa, Bacillus halodurans; identical to SEQ ID NO: 8

SEQ ID NO: 135 - DNA, 71 bp, Foot-and-mouth disease virus
  source 1..71: mol_type = other DNA
gcgccagtaaagcagacattaaactttgatttctgaaacttgcaggtgatgtagagtcaaatccaggtcca

SEQ ID NO: 136 - DNA, 72 bp, Foot-and-mouth disease virus
  source 1..72: mol_type = other DNA
ggcagcggaaaacagctgttgaattttgaccttctcaagttggcgggagacgtggagtccaacccagggccc

SEQ ID NO: 137 - DNA, 57 bp, unidentified
  source 1..57: mol_type = other DNA; note = porcine teschovirus-1 2A
gccactaacttctccctgttgaaacaagcaggggatgtcgaagagaatcccgggcca

SEQ ID NO: 138 - DNA, 60 bp, Equine rhinitis A virus
  source 1..60: mol_type = other DNA
caatgtactaactacgctttgttgaaactcgctggcgatgttgaaagtaaccccggtcct

SEQ ID NO: 139 - DNA, 72 bp, Thosea asigna virus
  source 1..72: mol_type = other DNA
ggcggcgggtccggaggagagggcagaggaagtcttctaacatgcggtgacgtggaggagaatcctggccca

SEQ ID NO: 140 - protein, 4 aa, synthetic construct
RKRR

SEQ ID NO: 141 - protein, 18 aa, synthetic construct
VSQTSKLTRAETVFPDVD

SEQ ID NO: 142 - protein, 10 aa, synthetic construct
EDVVCCSMSY

SEQ ID NO: 143 - protein, 6 aa, synthetic construct
RVLAEA

SEQ ID NO: 144 - protein, 17 aa, synthetic construct
GGGGSSPLGLWAGGGGS

SEQ ID NO: 145 - protein, 12 aa, synthetic construct
TRHRQPRGWEQL

SEQ ID NO: 146 - protein, 6 aa, synthetic construct
PLGLWA

SEQ ID NO: 147 - protein, 7 aa, synthetic construct
  VARIANT 7: note = X can be G or S
ENLYFQX

SEQ ID NO: 148 - protein, 7 aa, synthetic construct
LSGRDNH

SEQ ID NO: 149 - protein, 18 aa, synthetic construct
KRTADGSEFESPKKKRKV

SEQ ID NO: 150 - protein, 434 aa, Bacteroides sp.; identical to SEQ ID NO: 20

SEQ ID NO: 151 - protein, 328 aa, Treponema denticola; identical to SEQ ID NO: 21

SEQ ID NO: 152 - protein, 328 aa, Bordetella sp.; identical to SEQ ID NO: 18

SEQ ID NO: 153 - protein, 352 aa, unidentified; identical to SEQ ID NO: 19
  source 1..352: note = ANMV-1 virus

SEQ ID NO: 154 - protein, 346 aa, unidentified; identical to SEQ ID NO: 22
  source 1..346: note = archaeon GW2011_AR20

SEQ ID NO: 155 - protein, 834 aa, Saccharomyces cerevisiae; identical to SEQ ID NO: 16

SEQ ID NO: 156 - protein, 854 aa, Saccharomyces cerevisiae; identical to SEQ ID NO: 17

SEQ ID NO: 157 - protein, 560 aa, Feline immunodeficiency virus; identical to SEQ ID NO: 26

SEQ ID NO: 158 - protein, 596 aa, unidentified; identical to SEQ ID NO: 27
  source 1..596: note = Human Endogenous Retrovirus K

SEQ ID NO: 159 - protein, 609 aa, unidentified; identical to SEQ ID NO: 25
  source 1..609: note = Human foamy virus

SEQ ID NO: 160 - protein, 677 aa, unidentified; identical to SEQ ID NO: 23
  source 1..677: note = Baboon endogenous virus strain M7

SEQ ID NO: 161 - protein, 669 aa, Feline leukemia virus; identical to SEQ ID NO: 24

SEQ ID NO: 162 - protein, 677 aa, synthetic construct; identical to SEQ ID NO: 38

SEQ ID NO: 163 - protein, 426 aa, Eubacterium rectale
DTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTC

SEQ ID NO: 164 - protein, 9 aa, synthetic construct
  VARIANT 5: note = X can be any amino acid
GDVEXNPGP

SEQ ID NO: 165 - protein, 4 aa, synthetic construct
GSSG